(19) United States
(12) Patent Application Publication
(10) Pub. No.: US 2005/0239832 A1
(43) Pub. Date: Oct. 27, 2005

(54) Methods of treatment of amyloidosis using bi-cyclic aspartyl protease inhibitors

(76) Inventors: Varghese John, San Francisco, CA (US); Michel Maillard, Redwood City, CA (US); Lawrence Fang, Foster City, CA (US); John Tucker, San Diego, CA (US); Louis Brogley, Santa Cruz, CA (US); Jose Aquino, Daly City, CA (US); Simeon Bowers, Oakland, CA (US); Gary Probst, San Francisco, CA (US); Jay S. Tung, Belmont, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/074,828

(22) Filed: Mar. 9, 2005

Related U.S. Application Data
(60) Provisional application No. 60/551,051, filed on Mar. 9, 2004.
(60) Provisional application No. 60/551,050, filed on Mar. 9, 2004.
(60) Provisional application No. 60/575,828, filed on Jun. 2, 2004.
(60) Provisional application No. 60/576,008, filed on Jun. 2, 2004.
(60) Provisional application No. 60/591,966, filed on Jul. 29, 2004.
(60) Provisional application No. 60/591,926, filed on Jul. 29, 2004.
(60) Provisional application No. 60/614,059, filed on Sep. 30, 2004.
(60) Provisional application No. 60/614,034, filed on Sep. 30, 2004.

(51) Int. Cl. A61K 31/381; A61K 31/137; A61K 31/35; A61K 31/445

(52) U.S. Cl. 514/317; 514/447; 514/459; 514/649; 514/326; 514/444

(57) ABSTRACT

The invention relates to novel compounds and methods of treating diseases, disorders, and conditions associated with amyloidosis. Amyloidosis refers to a collection of diseases, disorders, and conditions associated with abnormal deposition of A-beta protein.

### CROSS-REFERENCE TO RELATED APPLICATIONS

This application claims the benefit of priority under 35 U.S.C. § 119(e) to U.S. Provisional Application 60/551,051 filed Mar. 9, 2004, U.S. Provisional Application 60/551,050 filed Mar. 9, 2004, U.S. Provisional Application 60/575,828 filed Jun. 2, 2004, U.S. Provisional Application 60/576,008 filed Jun. 2, 2004, U.S. Provisional Application 60/591,966 filed Jul. 29, 2004, U.S. Provisional Application 60/591,926 filed Jul. 29, 2004, U.S. Provisional Application 60/614,059 filed Sep. 30, 2004, and U.S. Provisional Application 60/614,034 filed Sep. 30, 2004.

### FIELD OF THE PRESENT INVENTION

The present invention is directed to novel compounds and also to methods of treating at least one condition, disorder, or disease associated with amyloidosis.

### BACKGROUND OF THE PRESENT INVENTION

Amyloidosis refers to a collection of at least one condition, disorder, or disease associated with abnormal deposition of amyloidal protein. For instance, Alzheimer's disease is believed to be caused by abnormal deposition of amyloidal protein in the brain. These amyloidal protein deposits, otherwise known as amyloid-beta peptide, A-beta, or betaA4, are the result of proteolytic cleavage of the amyloid precursor protein (APP).

The majority of APP molecules that undergo proteolytic cleavage are cleaved by the aspartyl protease alpha-secretase. Alpha-secretase cleaves APP between Lys687 and Leu688 producing a large, soluble fragment, alpha-sAPP, which is a secreted form of APP that does not result in beta-amyloid plaque formation. The alpha-secretase cleavage pathway precludes the formation of A-beta, thus providing an alternate target for preventing or treating amyloidosis.

Some APP molecules, however, are cleaved by a different aspartyl protease known as beta-secretase, which is also referred to in the literature as BACE, BACE1, Asp2, and Memapsin2. Beta-secretase cleaves APP after Met671, creating a C-terminal fragment. See, for example, Sinha et al., Nature, (1999), 402:537-554 and published PCT application WO 00/17369. After cleavage of APP by beta-secretase, an additional aspartyl protease, gamma-secretase, may then cleave the C-terminus of this fragment, at either Val711 or Ile713, found within the APP transmembrane domain, generating an A-beta peptide. The A-beta peptide may then proceed to form beta-amyloid plaques. A detailed description of the proteolytic processing of APP fragments is found, for example, in U.S. Pat. Nos. 5,441,870, 5,721,130, and 5,942,400.

The amyloidal disease Alzheimer's is a progressive degenerative disease that is characterized by two major pathologic observations in the brain which are (1) neurofibrillary tangles, and (2) beta-amyloid (or neuritic) plaques. A major factor in the development of Alzheimer's disease is A-beta deposits in regions of the brain responsible for cognitive activities. These regions include, for example, the hippocampus and cerebral cortex. A-beta is a neurotoxin that may be causally related to neuronal death observed in Alzheimer's disease patients. See, for example, Selkoe, Neuron, 6 (1991) 487. Since A-beta peptide accumulates as a result of APP processing by beta-secretase, inhibiting beta-secretase's activity is desirable for the treatment of Alzheimer's disease.

Dementia-characterized disorders also arise from A-beta accumulation in the brain including accumulation in cerebral blood vessels (known as vasculary amyloid angiopathy) such as in the walls of meningeal and parenchymal arterioles, small arteries, capillaries, and venules. A-beta may also be found in cerebrospinal fluid of both individuals with or without Alzheimer's disease. Additionally, neurofibrillary tangles similar to the ones observed in Alzheimer's patients can also be found in individuals without Alzheimer's disease. In this regard, a patient exhibiting symptoms of Alzheimer's due to A-beta deposits and neurofibrillary tangles in their cerebrospinal fluid may in fact be suffering from some other form of dementia. See, for example, Seubert et al., Nature, 359 (1992) 325-327. Examples of other forms of dementia where A-beta accumulation generates amyloidogenic plaques or results in vascular amyloid angiopathy include Trisomy 21 (Down's Syndrome), Hereditary Cerebral Hemorrhage with amyloidosis of the Dutch-Type (HCHWA-D), and other neurodegenerative disorders. Inhibiting beta-secretase is therefore not only desirable for the treatment of Alzheimer's, but also for the treatment of other conditions associated with amyloidosis.

Amyloidosis is also implicated in the pathophysiology of stroke. Cerebral amyloid angiopathy is a common feature of the brains of stroke patients exhibiting symptoms of dementia, focal neurological syndromes, or other signs of brain damage. See, for example, Corio et al., Neuropath Appl. Neurobiol., 22 (1996) 216-227. This suggests that production and deposition of A-beta may contribute to the pathology of Alzheimer's disease, stroke, and other diseases and conditions associated with amyloidosis. Accordingly, the inhibition of A-beta production is desirable for the treatment of Alzheimer's disease, stroke, and other diseases and conditions associated with amyloidosis.

Presently there are no known effective treatments for preventing, delaying, halting, or reversing the progression of Alzheimer's disease and other conditions associated with amyloidosis. Consequently, there is an urgent need for methods of treatment capable of preventing and treating conditions associated with amyloidosis including Alzheimer's disease.

Likewise, there is a need for compounds and methods of treatment that inhibit beta-secretase-mediated cleavage of APP. There is also a need for compounds and methods of treatment using compounds that are effective inhibitors of A-beta production, and/or are effective at reducing A-beta deposits or plaques, as well as methods of treatment capable of combating diseases and conditions characterized by amyloidosis, or A-beta deposits, or plaques.

There is also a need for methods of treating conditions associated with amyloidosis using compounds that are efficacious, bioavailable and/or selective for beta-secretase. An increase in efficacy, selectivity, and/or oral bioavailability may result in preferred, safer, less expensive products that are easier for patients to use.

There is also a need for methods of treating conditions associated with amyloidosis using compounds with characteristics that would allow them to cross the blood-brain barrier. Desirable characteristics include a low molecular weight and a high log P (increased log P=increased lipophilicity).

Generally, known aspartyl protease inhibitors are either incapable of crossing the blood-brain barrier or do so with great difficulty. Thus, these compounds are unsuitable for the treatment of the conditions described herein. Accordingly, there is a need for methods of treating conditions associated with amyloidosis using compounds that can readily cross the blood-brain barrier and inhibit beta-secretase.

There is also a need for a method of finding suitable compounds for inhibiting beta-secretase activity, inhibiting cleavage of APP, inhibiting production of A-beta, and/or reducing A-beta deposits or plaques.

The present invention is directed to compounds and methods of treating at least one condition, disorder, or disease associated with amyloidosis. An embodiment of the present invention is a method of administering at least one compound of formula (I) 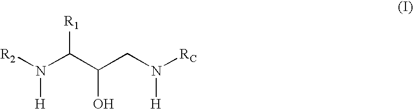
wherein R₁, R₂, and R_{C} are defined below, in treating at least one condition, disorder, or disease associated with amyloidosis. Another embodiment of the present invention is directed to methods of treatment comprising administering at least one compound of formula (I) wherein R₁, R₂, and R_{C} are defined below useful in preventing, delaying, halting, or reversing the progression of Alzheimer's disease.

Another embodiment of the present invention is directed to uses of beta-secretase inhibitors of at least one compound of formula (I) wherein R₁, R₂, and R_{C} are defined below in treating or preventing at least one condition, disorder, or disease associated with amyloidosis.

Another embodiment of the present invention is to administer beta-secretase inhibitors of at least one compound of formula (I) wherein R₁, R₂, and R_{C} are defined below, exhibiting at least one property chosen from improved efficacy, oral bioavailability, selectivity, and blood-brain barrier penetrating properties.

### BRIEF SUMMARY OF THE PRESENT INVENTION

The present invention is directed to methods and compounds useful in treating diseases, disorders, and conditions associated with amyloidosis. As previously noted, amyloidosis refers to a collection of diseases, disorders, and conditions associated with abnormal deposition of A-beta protein.

An embodiment of the present invention is to provide compounds having properties contributing to viable pharmaceutical compositions. These properties include improved efficacy, bioavailability, selectivity, and/or blood-brain barrier penetrating properties. They can be inter-related, though an increase in any one of them correlates to a benefit for the compound and its corresponding method of treatment. For example, an increase in any one of these properties may result in preferred, safer, less expensive products that are easier for patients to use.

In an embodiment, the present invention provides a method for preventing or treating conditions associated with amyloidosis, comprising administering to a patient in need thereof a therapeutically effective amount of at least one compound of formula (I), 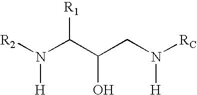
or a pharmaceutically acceptable salt thereof, wherein R₁, R₂, and R_{C} are as defined below.

In another embodiment, the present invention provides a method of preventing or treating conditions associated with amyloidosis, comprising administering to a host a composition comprising a therapeutically effective amount of at least one compound of formula (I), or a pharmaceutically acceptable salt thereof, wherein the inhibition is at least 10% for a dose ≦100 mg/kg, and wherein R₁, R₂, and R_{C} are as defined below.

In another embodiment, the present invention provides a method for preventing or treating conditions associated with amyloidosis, comprising administering to a patient in need thereof a therapeutically effective amount of at least one compound of formula (I), or a pharmaceutically acceptable salt thereof, the compound having an F value of at least 10%, wherein R₁, R₂, and R_{C} are as defined below.

In another embodiment, the present invention provides a method of preventing or treating conditions associated with amyloidosis, comprising administering to a host an composition comprising a therapeutically effective amount of at least one selective beta-secretase inhibitor of formula (I), or pharmaceutically acceptable salt thereof, wherein R₁, R₂, and R_{C} are as defined below.

In another embodiment, the present invention provides a method of preventing or treating Alzheimer's disease by administering to a host an effective amount of at least one compound of formula (I), or a pharmaceutically acceptable salt thereof, wherein R₁, R₂, and R_{C} are as defined below.

In another embodiment, the present invention provides a method of preventing or treating dementia by administering to a host an effective amount of at least one compound of formula (I), or pharmaceutically acceptable salt thereof, wherein R₁, R₂, and R_{C} are as defined below.

In another embodiment, the present invention provides a method of inhibiting beta-secretase activity in a host, the method comprising administering to the host an effective amount of at least one compound of formula (I), or a pharmaceutically acceptable salt thereof, wherein R₁, R₂, and R_{C} are as defined below.

In another embodiment, the present invention provides a method of inhibiting beta-secretase activity in a cell, the method comprising administering to the cell an effective amount of at least one compound of formula (I), or a pharmaceutically acceptable salt thereof, wherein R₁, R₂, and R_{C} are as defined below.

In another embodiment, the present invention provides a method of inhibiting beta-secretase activity in a host, the method comprising administering to the host an effective amount of at least one compound of formula (I), or a pharmaceutically acceptable salt thereof, wherein the host is a human, and wherein R₁, R₂, and R_{C} are as defined below.

In another embodiment, the present invention provides a method of affecting beta-secretase-mediated cleavage of amyloid precursor protein in a patient, comprising administering a therapeutically effective amount of at least one compound of formula (I), or a pharmaceutically acceptable salt thereof, wherein R₁, R₂, and R_{C} are as defined below.

In another embodiment, the present invention provides a method of inhibiting cleavage of amyloid precursor protein at a site between Met596 and Asp597 (numbered for the APP-695 amino acid isotype), or at a corresponding site of an isotype or mutant thereof, comprising administering a therapeutically effective amount of at least one compound of formula (I), or a pharmaceutically acceptable salt thereof, wherein R₁, R₂, and R_{C} are as defined below.

In another embodiment, the present invention provides a method of inhibiting production of A-beta, comprising administering to a patient a therapeutically effective amount of at least one compound of formula (I), or a pharmaceutically acceptable salt thereof, wherein R₁, R₂, and R_{C} are as defined below.

In another embodiment, the present invention provides a method of preventing or treating deposition of A-beta, comprising administering a therapeutically effective amount of at least one compound of formula (I), or a pharmaceutically acceptable salt thereof, wherein R₁, R₂, and R_{C} are as defined below.

In another embodiment, the present invention provides a method of preventing, delaying, halting, or reversing a disease characterized by A-beta deposits or plaques, comprising administering a therapeutically effective amount of at least one compound of formula (I), or a pharmaceutically acceptable salt thereof, wherein R₁, R₂, and R_{C} are as defined below.

In another embodiment, the A-beta deposits or plaques are in a human brain.

In another embodiment, the present invention provides a method of inhibiting the activity of at least one aspartyl protease in a patient in need thereof, comprising administering a therapeutically effective amount of at least one compound of formula (I), or a pharmaceutically acceptable salt thereof, wherein R₁, R₂, and R_{C} are as defined below.

In another embodiment, the at least one aspartyl protease is beta-secretase.

In another embodiment, the present invention provides a method of interacting an inhibitor with beta-secretase, comprising administering to a patient in need thereof a therapeutically effective amount of at least one compound of formula (I), or a pharmaceutically acceptable salt thereof, wherein R₁, R₂, and R_{C} are as defined below, wherein the at least one compound interacts with at least one beta-secretase subsite such as S1, S1′, or S2′.

In another embodiment, the present invention provides an article of manufacture, comprising (a) at least one dosage form of at least one compound of formula (I), or pharmaceutically acceptable salt thereof, wherein R₁, R₂, and R_{C} are defined below, (b) a package insert providing that a dosage form comprising a compound of formula (I) should be administered to a patient in need of therapy for at least one disorder, condition or disease associated with amyloidosis, and (c) at least one container in which at least one dosage form of at least one compound of formula (I) is stored.

In another embodiment, the present invention provides a packaged pharmaceutical composition for treating conditions related to amyloidosis, comprising (a) a container which holds an effective amount of at least one compound of formula (I), or a pharmaceutically acceptable salt thereof, wherein R₁, R₂, and R_{C} are as defined below, and (b) instructions for using the pharmaceutical composition.

### DEFINITIONS

Throughout the specification and claims, including the detailed description below, the following definitions apply.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

Where multiple substituents are indicated as being attached to a structure, the substituents can be the same or different.

APP, amyloid precursor protein, is defined as any APP polypeptide, including APP variants, mutations, and isoforms, for example, as disclosed in U.S. Pat. No. 5,766,846.

Beta-amyloid peptide (A-beta peptide) is defined as any peptide resulting from beta-secretase mediated cleavage of APP, including, for example, peptides of 39, 40, 41, 42, and 43 amino acids, and extending from the beta-secretase cleavage site to amino acids 39, 40, 41, 42, or 43.

Beta-secretase is an aspartyl protease that mediates cleavage of APP at the N-terminus of A-beta. Human beta-secretase is described, for example, in WO 00/17369.

The term “complex” as used herein refers to an inhibitor-enzyme complex, wherein the inhibitor is a compound of formula (I) described herein, and wherein the enzyme is beta-secretase or a fragment thereof.

The term “host” as used herein refers to a cell or tissue, in vitro or in vivo, an animal, or a human.

The term “treating” refers to administering a compound or a composition of formula (I) to a host having at least a tentative diagnosis of disease or condition. The methods of treatment and compounds of the present invention will delay, halt, or reverse the progression of the disease or condition thereby giving the host a longer and/or more functional life span.

The term “preventing” refers to administering a compound or a composition of formula (I) to a host who has not been diagnosed as having the disease or condition at the time of administration, but who could be expected to develop the disease or condition or be at increased risk for the disease or condition. The methods of treatment and compounds of the present invention may slow the development of disease symptoms, delay the onset of the disease or condition, halt the progression of disease development, or prevent the host from developing the disease or condition at all. Preventing also includes administration of a compound or a composition of the present invention to those hosts thought to be predisposed to the disease or condition due to age, familial history, genetic or chromosomal abnormalities, due to the presence of one or more biological markers for the disease or condition, such as a known genetic mutation of APP or APP cleavage products in brain tissues or fluids, and/or due to environmental factors.

The term “halogen” in the present invention refers to fluorine, bromine, chlorine, or iodine.

The term “alkyl” in the present invention refers to straight or branched chain alkyl groups having 1 to 20 carbon atoms. An alkyl group may optionally comprise at least one double bond and/or at least one triple bond. The alkyl groups herein are unsubstituted or substituted in one or more positions with various groups. For example, such alkyl groups may be optionally substituted with at least one group selected from alkyl, alkoxy, —C(O)H, -carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, aralkoxycarbonylamino, halogen, alkyl thio, alkylsulfinyl, alkylsulfonyl, hydroxy, cyano, nitro, amino, monoalkylamino, dialkylamino, halo alkyl, halo alkoxy, aminoalkyl, monoalkylaminoalkyl, dialkylaminoalkyl, and the like. Additionally, at least one carbon within any such alkyl may be optionally replaced with —C(O)—.

Examples of alkyls include methyl, ethyl, ethenyl, ethynyl, propyl, 1-ethyl-propyl, propenyl, propynyl, isopropyl, n-butyl, isobutyl, sec-butyl, tert-butyl, 2-methylbutyl, 3-methyl-butyl, 1-but-3-enyl, butynyl, pentyl, 2-pentyl, isopentyl, neopentyl, 3-methylpentyl, 1-pent-3-enyl, 1-pent-4-enyl, pentyn-2-yl, hexyl, 2-hexyl, 3-hexyl, 1-hex-5-enyl, formyl, acetyl, acetylamino, trifluoromethyl, propionic acid ethyl ester, trifluoroacetyl, methylsulfonyl, ethylsulfonyl, 1-hydroxy-1-methylethyl, 2-hydroxy-1,1-dimethyl-ethyl, 1,1-dimethyl-propyl, cyano-dimethyl-methyl, propylamino, and the like.

In an embodiment, alkyls may be selected from the group comprising sec-butyl, isobutyl, ethynyl, 1-ethyl-propyl, pentyl, 3-methyl-butyl, pent-4-enyl, isopropyl, tert-butyl, 2-methylbutane, and the like.

In another embodiment, alkyls may be selected from formyl, acetyl, acetylamino, trifluoromethyl, propionic acid ethyl ester, trifluoroacetyl, methylsulfonyl, ethylsulfonyl, 1-hydroxy-1-methylethyl, 2-hydroxy-1,1-dimethyl-ethyl, 1,1-dimethyl-propyl, cyano-dimethyl-methyl, propylamino, and the like.

The term “alkoxy” in the present invention refers to straight or branched chain alkyl groups, wherein an alkyl group is as defined above, and having 1 to 20 carbon atoms, attached through at least one divalent oxygen atom, such as, for example, methoxy, ethoxy, propoxy, isopropoxy, n-butoxy, sec-butoxy, tert-butoxy, pentoxy, isopentoxy, neopentoxy, hexyloxy, heptyloxy, allyloxy, 2-(2-methoxy-ethoxy)-ethoxy, benzyloxy, 3-methylpentoxy, and the like.

In an embodiment, alkoxy groups may be selected from the group comprising allyloxy, hexyloxy, heptyloxy, 2-(2-methoxy-ethoxy)-ethoxy, benzyloxy, and the like.

The term “—C(O)-alkyl” or “alkanoyl” refers to an acyl radical derived from an alkylcarboxylic acid, a cycloalkylcarboxylic acid, a heterocycloalkylcarboxylic acid, an arylcarboxylic acid, an arylalkylcarboxylic acid, a heteroarylcarboxylic acid, or a heteroarylalkylcarboxylic acid, examples of which include formyl, acetyl, 2,2,2-trifluoroacetyl, propionyl, butyryl, valeryl, 4-methylvaleryl, and the like.

The term “cycloalkyl” refers to an optionally substituted carbocyclic ring system of one or more 3, 4, 5, 6, 7, or 8 membered rings. A cycloalkyl can further include 9, 10, 11, 12, 13, and 14 membered fused ring systems. A cycloalkyl can be saturated or partially unsaturated. A cycloalkyl may be monocyclic, bicyclic, tricyclic, and the like. Bicyclic and tricyclic as used herein are intended to include both fused ring systems, such as adamantyl, octahydroindenyl, decahydro-naphthyl, and the like, substituted ring systems, such as cyclopentylcyclohexyl, and spirocycloalkyls such as spiro[2.5]octane, spiro[4.5]decane, 1,4-dioxa-spiro[4.5]decane, and the like. A cycloalkyl may optionally be a benzo fused ring system, which is optionally substituted as defined herein with respect to the definition of aryl. At least one —CH₂— group within any such cycloalkyl ring system may be optionally replaced with —C(O)—, —C(S)—, —C(═N—H)—, —C(═N—OH)—, —C(═N-alkyl)- (optionally substituted as defined herein with respect to the definition of alkyl), or —C(═N—O-alkyl)- (optionally substituted as defined herein with respect to the definition of alkyl).

Further examples of cycloalkyl radicals include cyclopropyl, cyclobutyl, cyclopentyl, cyclohexyl, octahydronaphthyl, 2,3-dihydro-1H-indenyl, and the like.

In one embodiment, a cycloalkyl may be selected from the group comprising cyclopentyl, cyclohexyl, cycloheptyl, adamantenyl, bicyclo[2.2.1]heptyl, and the like.

The cycloalkyl groups herein are unsubstituted or substituted in at least one position with various groups. For example, such cycloalkyl groups may be optionally substituted with alkyl, alkoxy, —C(O)H, carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, aralkoxycarbonylamino, halogen, alkylthio, alkylsulfinyl, alkylsulfonyl, hydroxy, cyano, nitro, amino, monoalkylamino, dialkylamino, haloalkyl, haloalkoxy, aminoalkyl, monoalkylaminoalkyl, dialkylaminoalkyl, and the like.

The term “cycloalkylcarbonyl” refers to an acyl radical of the formula cycloalkyl-C(O)— in which the term “cycloalkyl” has the significance given above, such as cyclopropylcarbonyl, cyclohexylcarbonyl, adamantylcarbonyl, 1,2,3,4-tetrahydro-2-naphthoyl, 2-acetamido-1,2,3,4-tetrahydro-2-naphthoyl, 1-hydroxy-1,2,3,4-tetrahydro-6-naphthoyl, and the like.

The term “heterocycloalkyl,” “heterocycle,” or “heterocyclyl” refers to a monocyclic, bicyclic or tricyclic heterocycle radical, containing at least one nitrogen, oxygen or sulfur atom ring member and having 3 to 8 ring members in each ring, wherein at least one ring in the heterocycloalkyl ring system may optionally contain at least one double bond. At least one —CH₂— group within any such heterocycloalkyl ring system may be optionally replaced with —C(O)—, —C(S)—, —C(═N—H)—, —C(═N—OH)—, —C(═N-alkyl)- (optionally substituted as defined herein with respect to the definition of alkyl), or —C(═N—O-alkyl)- (optionally substituted as defined herein with respect to the definition of alkyl).

The terms “bicyclic” and “tricyclic” as used herein are intended to include both fused ring systems, such as 2,3-dihydro-1H-indole, and substituted ring systems, such as bicyclohexyl. At least one —CH₂— group within any such heterocycloalkyl ring system may be optionally replaced with —C(O)—, —C(N)— or —C(S)—. Heterocycloalkyl is intended to include sulfones, sulfoxides, N-oxides of tertiary nitrogen ring members, and carbocyclic fused and benzo fused ring systems wherein the benzo fused ring system is optionally substituted as defined herein with respect to the definition of aryl. Such heterocycloalkyl radicals may be optionally substituted on one or more carbon atoms by halogen, alkyl, alkoxy, cyano, nitro, amino, alkylamino, dialkylamino, monoalkylaminoalkyl, dialkylaminoalkyl, haloalkyl, haloalkoxy, aminohydroxy, oxo, aryl, aralkyl, heteroaryl, heteroaralkyl, amidino, N-alkylamidino, alkoxycarbonylamino, alkylsulfonylamino, and the like, and/or on a secondary nitrogen atom (i.e., —NH—) by hydroxy, alkyl, aralkoxycarbonyl, alkanoyl, heteroaralkyl, phenyl, phenylalkyl, and the like.

Examples of a heterocycloalkyl include morpholinyl, thiomorpholinyl, thiomorpholinyl S-oxide, thiomorpholinyl S,S-dioxide, piperazinyl, homopiperazinyl, pyrrolidinyl, pyrrolinyl, 2,5-dihydro-pyrrolyl, tetrahydropyranyl, pyranyl, thiopyranyl, piperidinyl, tetrahydrofuranyl, tetrahydrothienyl, imidazolidinyl, homopiperidinyl, 1,2-dihyrdo-pyridinyl, homomorpholinyl, homothiomorpholinyl, homothiomorpholinyl S,S-dioxide, oxazolidinonyl, dihydropyrazolyl, dihydropyrrolyl, 1,4-dioxa-spiro[4.5]decyl, dihydropyrazinyl, dihydropyridinyl, dihydropyrimidinyl, dihydrofuryl, dihydropyranyl, tetrahydrothienyl S-oxide, tetrahydrothienyl S,S-dioxide, homothiomorpholinyl S-oxide, 2-oxo-piperidinyl, 5-oxo-pyrrolidinyl, 2-oxo-1,2-dihydro-pyridinyl, 6-oxo-6H-pyranyl, 1,1-dioxo-hexahydro-thiopyranyl, 1-acetyl-piperidinyl, 1-methanesulfonylpiperidinyl, 1-ethanesulfonylpiperidinyl, 1-oxo-hexahydro-thiopyranyl, 1-(2,2,2-trifluoroacetyl)-piperidinyl, 1-formyl-piperidinyl, and the like.

In an embodiment, a heterocycloalkyl may be selected from pyrrolidinyl, 2,5-dihydro-pyrrolyl, piperidinyl, 1,2-dihyrdo-pyridinyl, pyranyl, piperazinyl, imidazolidinyl, thiopyranyl, tetrahydropyranyl, 1,4-dioxa-spiro[4.5]decyl, and the like.

In another embodiment, a heterocycloalkyl may be selected from 2-oxo-piperidinyl, 5-oxo-pyrrolidinyl, 2-oxo-1,2-dihydro-pyridinyl, 6-oxo-6H-pyranyl, 1,1-dioxo-hexahydro-thiopyranyl, 1-acetyl-piperidinyl, 1-methanesulfonyl piperidinyl, 1-ethanesulfonylpiperidinyl, 1-oxo-hexahydro-thiopyranyl, 1-(2,2,2-trifluoroacetyl)-piperidinyl, 1-formyl-piperidinyl, and the like.

The term “aryl” refers to an aromatic carbocyclic group having a single ring (e.g., phenyl) or multiple condensed rings in which at least one ring is aromatic. The aryl may be monocyclic, bicyclic, tricyclic, etc. Bicyclic and tricyclic as used herein are intended to include both fused ring systems, such as naphthyl and β-carbolinyl, and substituted ring systems, such as biphenyl, phenylpyridyl, diphenylpiperazinyl, tetrahydronaphthyl, and the like. Preferred aryl groups of the present invention are phenyl, 1-naphthyl, 2-naphthyl, indanyl, indenyl, dihydronaphthyl, fluorenyl, tetralinyl or 6,7,8,9-tetrahydro-5H-benzo[a]cycloheptenyl. The aryl groups herein are unsubstituted or substituted in one or more positions with various groups. For example, such aryl groups may be optionally substituted with alkyl, alkoxy, —C(O)H, carboxy, alkoxycarbonyl, aryl, heteroaryl, cycloalkyl, heterocycloalkyl, amido, alkanoylamino, amidino, alkoxycarbonylamino, N-alkyl amidino, N-alkyl amido, N,N′-dialkylamido, aralkoxycarbonylamino, halogen, alkyl thio, alkylsulfinyl, alkylsulfonyl, hydroxy, cyano, nitro, amino, monoalkylamino, dialkylamino, halo alkyl, halo alkoxy, aminoalkyl, monoalkylaminoalkyl, dialkylaminoalkyl, and the like.

Examples of aryl radicals are phenyl, p-tolyl, 4-methoxyphenyl, 4-(tert-butoxy)phenyl, 3-methyl-4-methoxyphenyl, 4-CF₃-phenyl, 4-fluorophenyl, 4-chlorophenyl, 3-nitrophenyl, 3-aminophenyl, 3-acetamidophenyl, 4-acetamidophenyl, 2-methyl-3-acetamidophenyl, 2-methyl-3-aminophenyl, 3-methyl-4-aminophenyl, 2-amino-3-methylphenyl, 2,4-dimethyl-3-aminophenyl, 4-hydroxyphenyl, 3-methyl-4-hydroxyphenyl, 1-naphthyl, 2-naphthyl, 3-amino-1-naphthyl, 2-methyl-3-amino-1-naphthyl, 6-amino-2-naphthyl, 4,6-dimethoxy-2-naphthyl, piperazinylphenyl, and the like.

Further examples of aryl radicals include 3-tert-butyl-1-fluoro-phenyl, 1,3-difluoro-phenyl, (1-hydroxy-1-methyl-ethyl)-phenyl, 1-fluoro-3-(2-hydroxy-1,1-dimethyl-ethyl)-phenyl, (1,1-dimethyl-propyl)-phenyl, cyclobutyl-phenyl, pyrrolidin-2-yl-phenyl, (5-oxo-pyrrolidin-2-yl)-phenyl, (2,5-dihydro-1H-pyrrol-2-yl)-phenyl, (1H-pyrrol-2-yl)-phenyl, (cyano-dimethyl-methyl)-phenyl, tert-butyl-phenyl, 1-fluoro-2-hydroxy-phenyl, 1,3-difluoro-4-propylamino-phenyl, 1,3-difluoro-4-hydroxy-phenyl, 1,3-difluoro-4-ethylamino-phenyl, 3-isopropyl-phenyl, (3H-[1,2,3]triazol-4-yl)-phenyl, [1,2,3]triazol-1-yl-phenyl, [1,2,4]thiadiazol-3-yl-phenyl, [1,2,4]thiadiazol-5-yl-phenyl, (4H-[1,2,4]triazol-3-yl)-phenyl, [1,2,4]oxadiazol-3-yl-phenyl, imidazol-1-yl-phenyl, (3H-imidazol-4-yl)-phenyl, [1,2,4]triazol-4-yl-phenyl, [1,2,4]oxadiazol-5-yl-phenyl, isoxazol-3-yl-phenyl, (1-methyl-cyclopropyl)-phenyl, isoxazol-4-yl-phenyl, isoxazol-5-yl-phenyl, 1-cyano-2-tert-butyl-phenyl, 1-trifluoromethyl-2-tert-butyl-phenyl, 1-chloro-2-tert-butyl-phenyl, 1-acetyl-2-tert-butyl-phenyl, 1-tert-butyl-2-methyl-phenyl, 1-tert-butyl-2-ethyl-phenyl, 1-cyano-3-tert-butyl-phenyl, 1-trifluoromethyl-3-tert-butyl-phenyl, 1-chloro-3-tert-butyl-phenyl, 1-acetyl-3-tert-butyl-phenyl, 1-tert-butyl-3-methyl-phenyl, 1-tert-butyl-3-ethyl-phenyl, 4-tert-butyl-1-imidazol-1-yl-phenyl, ethylphenyl, isobutylphenyl, isopropylphenyl, 3-allyloxy-1-fluoro-phenyl, (2,2-dimethyl-propyl)-phenyl, ethynylphenyl, 1-fluoro-3-heptyloxy-phenyl, 1-fluoro-3-[2-(2-methoxy-ethoxy)-ethoxy]-phenyl, 1-benzyloxy-3-fluoro-phenyl, 1-fluoro-3-hydroxy-phenyl, 1-fluoro-3-hexyloxy-phenyl, (4-methyl-thiophen-2-yl)-phenyl, (5-acetyl-thiophen-2-yl)-phenyl, furan-3-yl-phenyl, thiophen-3-yl-phenyl, (5-formyl-thiophen-2-yl)-phenyl, (3-formyl-furan-2-yl)-phenyl, acetylamino-phenyl, trifluoromethylphenyl, sec-butyl-phenyl, pentylphenyl, (3-methyl-butyl)-phenyl, (1-ethyl-propyl)-phenyl, cyclopentyl-phenyl, 3-pent-4-enyl-phenyl, phenyl propionic acid ethyl ester, pyridin-2-yl-phenyl, (3-methyl-pyridin-2-yl)-phenyl, thiazol-2-yl-phenyl, (3-methyl-thiophen-2-yl)-phenyl, fluoro-phenyl, adamantan-2-yl-phenyl, 1,3-difluoro-2-hydroxy-phenyl, cyclopropyl-phenyl, 1-bromo-3-tert-butyl-phenyl, (3-bromo-[1,2,4]thiadiazol-5-yl)-phenyl, (1-methyl-1H-imidazol-2-yl)-phenyl, (3,5-dimethyl-3H-pyrazol-4-yl)-phenyl, (3,6-dimethyl-pyrazin-2-yl)-phenyl, (3-cyano-pyrazin-2-yl)-phenyl, thiazol-4-yl-phenyl, (4-cyano-pyridin-2-yl)-phenyl, pyrazin-2-yl-phenyl, (6-methyl-pyridazin-3-yl)-phenyl, (2-cyano-thiophen-3-yl)-phenyl, (2-chloro-thiopen-3-yl)-phenyl, (5-acetyl-thiophen-3-yl)-phenyl, cyano-phenyl, and the like.

The term “heteroaryl” refers to an aromatic heterocycloalkyl radical as defined above. The heteroaryl groups herein are unsubstituted or substituted in at least one position with various groups. For example, such heteroaryl groups may be optionally substituted with, for example, alkyl, alkoxy, halogen, hydroxy, cyano, nitro, amino, monoalkylamino, dialkylamino, haloalkyl, haloalkoxy, —C(O)H, carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, alkyl thio, alkylsulfinyl, alkylsulfonyl, aralkoxycarbonylamino, aminoalkyl, monoalkylaminoalkyl, dialkylaminoalkyl, and the like.

Examples of heteroaryl groups include pyridyl, pyrimidyl, furanyl, imidazolyl, thienyl, oxazolyl, thiazolyl, pyrazinyl, 3-methyl-thienyl, 4-methyl-thienyl, 3-propyl-thienyl, 2-chloro-thienyl, 2-chloro-4-ethyl-thienyl, 2-cyano-thienyl, 5-acetyl-thienyl, 5-formyl-thienyl, 3-formyl-furanyl, 3-methyl-pyridinyl, 3-bromo-[1,2,4]thiadiazolyl, 1-methyl-1H-imidazole, 3,5-dimethyl-3H-pyrazolyl, 3,6-dimethyl-pyrazinyl, 3-cyano-pyrazinyl, 4-tert-butyl-pyridinyl, 4-cyano-pyridinyl, 6-methyl-pyridazinyl, 2-tert-butyl-pyrimidinyl, 4-tert-butyl-pyrimidinyl, 6-tert-butyl-pyrimidinyl, 5-tert-butyl-pyridazinyl, 6-tert-butyl-pyridazinyl, quinolinyl, benzothienyl, indolyl, indolinyl, pyridazinyl, isoindolyl, isoquinolyl, quinazolinyl, quinoxalinyl, phthalazinyl, imidazolyl, isoxazolyl, pyrazolyl, indolizinyl, indazolyl, benzothiazolyl, benzimidazolyl, benzofuranyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide, tetrahydrocarbazole, tetrahydrobetacarboline, and the like.

In an embodiment, a heteroaryl group may be selected from pyridyl, pyrimidyl, furanyl, imidazolyl, thienyl, oxazolyl, thiazolyl, pyrazinyl, and the like.

In another embodiment, a heteroaryl group may be selected from 3-methyl-thienyl, 4-methyl-thienyl, 3-propyl-thienyl, 2-chloro-thienyl, 2-chloro-4-ethyl-thienyl, 2-cyano-thienyl, 5-acetyl-thienyl, 5-formyl-thienyl, 3-formyl-furanyl, 3-methyl-pyridinyl, 3-bromo-[1,2,4]thiadiazolyl, 1-methyl-1H-imidazole, 3,5-dimethyl-3H-pyrazolyl, 3,6-dimethyl-pyrazinyl, 3-cyano-pyrazinyl, 4-tert-butyl-pyridinyl, 4-cyano-pyridinyl, 6-methyl-pyridazinyl, 2-tert-butyl-pyrimidinyl, 4-tert-butyl-pyrimidinyl, 6-tert-butyl-pyrimidinyl, 5-tert-butyl-pyridazinyl, 6-tert-butyl-pyridazinyl, and the like.

Further examples of heterocycloalkyls and heteroaryls may be found in Katritzky, A. R. et al., Comprehensive Heterocyclic Chemistry. The Structure, Reactions, Synthesis and Use of Heterocyclic Compounds, Vol. 1-8, New York: Pergamon Press, 1984.

The term “aralkoxycarbonyl” refers to a radical of the formula aralkyl-O—C(O)— in which the term “aralkyl” is encompassed by the definitions above for aryl and alkyl. Examples of an aralkoxycarbonyl radical include benzyloxycarbonyl, 4-methoxyphenylmethoxycarbonyl, and the like.

The term “aryloxy” refers to a radical of the formula —O-aryl in which the term aryl is as defined above.

The term “aralkanoyl” refers to an acyl radical derived from an aryl-substituted alkanecarboxylic acid such as phenylacetyl, 3-phenylpropionyl(hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like.

The term “aroyl” refers to an acyl radical derived from an arylcarboxylic acid, “aryl” having the meaning given above. Examples of such aroyl radicals include substituted and unsubstituted benzoyl or naphthoyl such as benzoyl, 4-chlorobenzoyl, 4-carboxybenzoyl, 4-(benzyloxycarbonyl)benzoyl, 1-naphthoyl, 2-naphthoyl, 6-carboxy-2 naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The term “haloalkyl” refers to an alkyl radical having the meaning as defined above wherein one or more hydrogens are replaced with a halogen. Examples of such haloalkyl radicals include chloromethyl, 1-bromoethyl, fluoromethyl, difluoromethyl, trifluoromethyl, 1,1,1-trifluoroethyl, and the like.

The term “epoxide” refers to chemical compounds or reagents comprising a bridging oxygen wherein the bridged atoms are also bonded to one another either directly or indirectly. Examples of epoxides include epoxyalkyl (e.g., ethylene oxide and 1,2-epoxybutane), epoxycycloalkyl (e.g., 1,2-epoxycyclohexane and 1,2-epoxy-1-methylcyclohexane), and the like.

The term “structural characteristics” refers to chemical moieties, chemical motifs, and portions of chemical compounds. These include R groups, such as those defined herein, ligands, appendages, and the like. For example, structural characteristics may be defined by their properties, such as, but not limited to, their ability to participate in intermolecular interactions, including Van der Waal's (e.g., electrostatic interactions, dipole-dipole interactions, dispersion forces, hydrogen bonding, and the like). Such characteristics may impart desired pharmacokinetic properties and thus have an increased ability to cause the desired effect and thus prevent or treat the targeted diseases or conditions.

Compounds of formula (I) also comprise structural moieties that participate in inhibitory interactions with at least one subsite of beta-secretase. For example, at least one moiety of the compounds of formula (I) may interact with at least one of the S1, S1′, and S2′ subsites, wherein S1 comprises residues Leu30, Tyr71, Phe108, Ile110, and Trp115, S1′ comprises residues Tyr198, Ile226, Val227, Ser 229, and Thr231, and S2′ comprises residues Ser35, Asn37, Pro70, Tyr71, Ile118, and Arg128. Such compounds and methods of treatment may have an increased ability to cause the desired effect and thus prevent or treat the targeted diseases or conditions.

The term “pharmaceutically acceptable” refers to those properties and/or substances that are acceptable to the patient from a pharmacological/toxicological point of view, and to the manufacturing pharmaceutical chemist from a physical/chemical point of view regarding composition, formulation, stability, patient acceptance and bioavailability.

The term “effective amount” as used herein refers to an amount of a therapeutic agent administered to a host, as defined herein, necessary to achieve a desired effect.

The term “therapeutically effective amount” as used herein refers to an amount of a therapeutic agent administered to a host to treat or prevent a condition treatable by administration of a composition of the invention. That amount is the amount sufficient to reduce or lessen at least one symptom of the disease being treated or to reduce or delay onset of one or more clinical markers or symptoms of the disease.

The term “therapeutically active agent” refers to a compound or composition that is administered to a host, either alone or in combination with another therapeutically active agent, to treat or prevent a condition treatable by administration of a composition of the invention.

The term “pharmaceutically acceptable salt” and “salt thereof” refer to acid addition salts or base addition salts of the compounds in the present invention. A pharmaceutically acceptable salt is any salt which retains the activity of the parent compound and does not impart any deleterious or undesirable effect on the subject to whom it is administered and in the context in which it is administered. Pharmaceutically acceptable salts include salts of both inorganic and organic acids. Pharmaceutically acceptable salts include acid salts such as acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycolylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic, toluenesulfonic, and the like. Other acceptable salts may be found, for example, in Stahl et al., Pharmaceutical Salts: Properties, Selection, and Use, Wiley-VCH; 1st edition (Jun. 15, 2002).

In another embodiment of the present invention, a pharmaceutically acceptable salt is selected from hydrochloric, hydrobromic, hydroiodic, nitric, sulfuric, phosphoric, citric, methanesulfonic, CH₃—(CH₂)₀₋₄—COOH, HOOC—(CH₂)₀₋₄—COOH, HOOC—CH═CH—COOH, phenyl-COOH, and the like.

The term “unit dosage form” refers to physically discrete units suitable as unitary dosages for human subjects or other mammals, each unit containing a predetermined quantity of active material calculated to produce the desired therapeutic effect, in association with a suitable pharmaceutical vehicle. The concentration of active compound in the drug composition will depend on absorption, inactivation, and/or excretion rates of the active compound, the dosage schedule, the amount administered and medium and method of administration, as well as other factors known to those of skill in the art.

The term “modulate” refers to a chemical compound's activity to either enhance or inhibit a functional property of biological activity or process.

The terms “interact” and “interactions” refer to a chemical compound's association and/or reaction with another chemical compound, such as an interaction between an inhibitor and beta-secretase. Interactions include, but are not limited to, hydrophobic, hydrophilic, lipophilic, lipophobic, electrostatic, and van der Waal's interactions, and hydrogen bonding.

An “article of manufacture” as used herein refers to materials useful for the diagnosis, prevention or treatment of the disorders described above, such as a container with a label. The label can be associated with the article of manufacture in a variety of ways including, for example, the label may be on the container or the label may be in the container as a package insert. Suitable containers include, for example, blister packs, bottles, bags, vials, syringes, test tubes, and the like. The containers may be formed from a variety of materials such as glass, metal, plastic, rubber, paper, and the like. The container holds a composition as described herein which is effective for diagnosing, preventing, or treating a condition treatable by a compound or composition of the present invention.

The article of manufacture may contain bulk quantities or less of a composition as described herein. The label on, or associated with, the container may provide instructions for the use of the composition in diagnosing, preventing, or treating the condition of choice, instructions for the dosage amount and for the methods of administration. The label may further indicate that the composition is to be used in combination with one or more therapeutically active agents wherein the therapeutically active agent is selected from an antioxidant, an anti-inflammatory, a gamma-secretase inhibitor, a neurotropic agent, an acetyl cholinesterase inhibitor, a statin, an A-beta, an anti-A-beta antibody, and/or a beta-secretase complex or fragment thereof.

The article of manufacture may further comprise multiple containers, also referred to herein as a kit, comprising a therapeutically active agent or a pharmaceutically-acceptable buffer, such as phosphate-buffered saline, Ringer's solution and/or dextrose solution. It may further include other materials desirable from a commercial and user standpoint, including other buffers, diluents, filters, needles, syringes, and/or package inserts with instructions for use.

The compounds of formula (I), their compositions, and methods of treatment employing them, can be enclosed in multiple or single dose containers. The enclosed compounds and/or compositions can be provided in kits, optionally including component parts that can be assembled for use. For example, a compound inhibitor in lyophilized form and a suitable diluent may be provided as separated components for combination prior to use. A kit may include a compound inhibitor and at least one additional therapeutic agent for co-administration. The inhibitor and additional therapeutic agents may be provided as separate component parts.

A kit may include a plurality of containers, each container holding at least one unit dose of the compound of the present invention. The containers are preferably adapted for the desired mode of administration, including, for example, pill, tablet, capsule, powder, gel or gel capsule, sustained-release capsule, or elixir form, and/or combinations thereof and the like for oral administration, depot products, pre-filled syringes, ampoules, vials, and the like for parenteral administration, and patches, medipads, creams, and the like for topical administration.

The term “Cₘₐₓ” refers to the peak plasma concentration of a compound in a host.

The term “Tₘₐₓ” refers to the time at peak plasma concentration of a compound in a host.

The term “half-life” refers to the period of time required for the concentration or amount of a compound in a host to be reduced to exactly one-half of a given concentration or amount.

### DETAILED DESCRIPTION OF THE PRESENT INVENTION

The present invention is directed to compounds and methods useful in treating diseases, disorders, and conditions characterized by amyloidosis. Amyloidosis refers to a collection of diseases, disorders, and conditions associated with abnormal deposition of amyloidal protein.

Accordingly, an aspect of the present invention is to provide a method of preventing or treating conditions which benefit from inhibition of at least one aspartyl-protease, comprising administering to a host a composition comprising a therapeutically effective amount of at least one compound of formula (I), 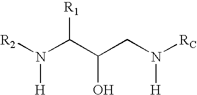
or a pharmaceutically acceptable salt thereof; wherein R₁ is selected from 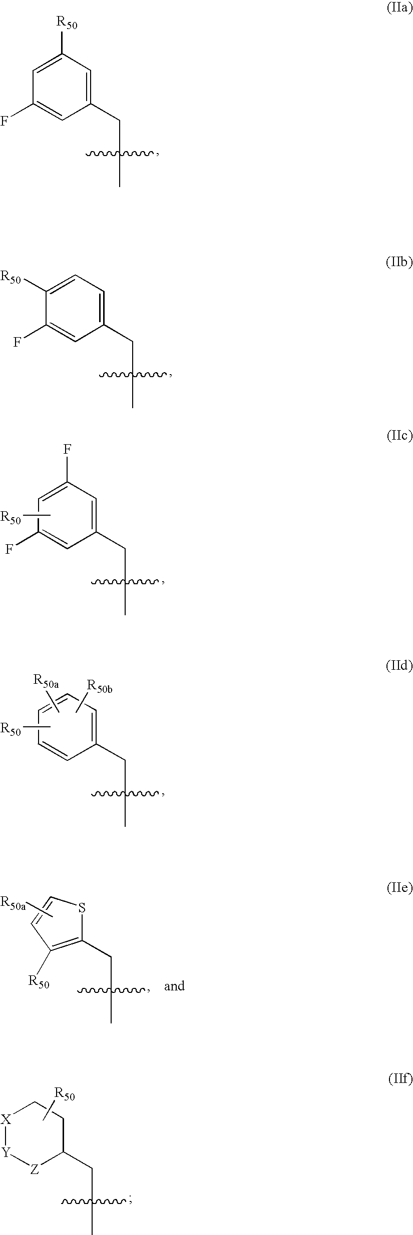
wherein X, Y, and Z are independently selected from —C(H)₀₋₂—, —O—, —C(O)—, —NH—, and —N—; wherein at least one bond of the (IIf) ring may optionally be a double bond; R₅₀, R₅₀ₐ, and R_{50b} are independently selected from —H, -halogen, —OH, —SH, —CN, —C(O)-alkyl, —NR₇R₈, —S(O)₀₋₂-alkyl, -alkyl, -alkoxy, —O-benzyl (optionally substituted with at least one substituent independently selected from —H, —OH, and alkyl), —C(O)—NR₇R₈, -alkyloxy, -alkoxyalkoxyalkoxy, and -cycloalkyl; wherein the alkyl, alkoxy, and cycloalkyl groups within R₅₀, R₅₀ₐ, and R_{50b} are optionally substituted with at least one substituent independently selected from alkyl, halogen, —OH, —NR₅R₆, —NR₇R₈, —CN, haloalkoxy, and alkoxy; R₅ and R₆ are independently selected from —H and alkyl; or R₅ and R₆, and the nitrogen to which they are attached, form a 5 or 6 membered heterocycloalkyl ring; R₇ and R₈ are independently selected from —H, -alkyl (optionally substituted with at least one group independently selected from —OH, —NH₂, and halogen), -cycloalkyl, and -alkyl-O-alkyl; R₂ is selected from —C(O)—CH₃, —C(O)—CH₂(halogen), —C(O)—CH(halogen)₂, 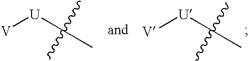
U is selected from —C(O)—, —C(═S)—, —S(O)₀₋₂—, —C═N—R₂₁—, —C═N—OR₂₁—, —C(O)—NR₂₀—, —C(O)—O—, —S(O)₂—NR₂₀—, and —S(O)₂—O—; U′ is selected from —C(O)—, —C═N—R₂₁—, —C═N—OR₂₁—, —C(O)—NR₂₀—, and —C(O)—O—; V is selected from aryl, heteroaryl, cycloalkyl, heterocycloalkyl, —[C(R₄)(R₄)]₁₋₃-D, and -(T)₀₋₁-R_{N}; V′ is selected from -(T)₀₋₁-R_{N ′}; wherein the aryl, heteroaryl, cycloalkyl, and heterocycloalkyl groups included within V and V′ are optionally substituted with 1 or 2 R_{B} groups; wherein at least one carbon of the aryl, heteroaryl, cycloalkyl, and heterocycloalkyl groups included within V and V′ are optionally replaced with —N—, —O—, —NH—, —C(O)—, —C(S)—, —C(═N—H)—, —C(═N—OH)—, —C(═N-alkyl)-, or —C(═N—O-alkyl)-; R_{B} at each occurrence is independently selected from halogen, —OH, —CF₃, —OCF₃, —O-aryl, —CN, —NR₁₀₁R′₁₀₁, alkyl, alkoxy, —(CH₂)₀₋₄—(C(O))₀₋₁—(O)₀₋₁-alkyl, —C(O)—OH, —(CH₂)₀₋₃-cycloalkyl, aryl, heteroaryl, and heterocycloalkyl; wherein, the alkyl, alkoxy, cycloalkyl, aryl, heteroaryl, or heterocycloalkyl groups included within R_{B} are optionally substituted with 1 or 2 groups independently selected from —C₁-C₄ alkyl, —C₁-C₄ alkoxy, —C₁-C₄ haloalkyl, —C₁-C₄ haloalkoxy, -halogen, —OH, —CN, and —NR₁₀₁R′₁₀₁; R₁₀₁ and R′₁₀₁ are independently selected from —H, -alkyl, —(C(O))₀₋₁—(O)₀₋₁-alkyl, —C(O)—OH, and -aryl; R₄ and R_{4′} are independently selected from -hydrogen, -alkyl, —(CH₂)₀₋₃-cycloalkyl, —(CH₂)₀₋₃—OH, -fluorine, —CF₃, —OCF₃, —O-aryl, -alkoxy, —C₃-C₇ cycloalkoxy, -aryl, and -heteroaryl, or R₄ and R_{4′} are taken together with the carbon to which they are attached to form a 3, 4, 5, 6, or 7 membered carbocyclic ring wherein 1, 2, or 3 carbons of the ring is optionally replaced with —O—, —N(H)—, —N(alkyl)-, —N(aryl)-, —C(O)—, or —S(O)₀₋₂; D is selected from aryl, heteroaryl, cycloalkyl, and heterocycloalkyl, wherein the aryl, heteroaryl, cycloalkyl, and heterocycloalkyl are optionally substituted with 1 or 2 R_{B} groups; T is selected from —NR₂₀— and —O—; R₂₀ is selected from H, —CN, -alkyl, -haloalkyl, and -cycloalkyl; R₂₁ is selected from —H, -alkyl, -haloalkyl, and -cycloalkyl; R_{N} is selected from —OH, —NH₂, —NH(alkyl), —NH(cycloalkyl), —N(alkyl)(alkyl), —N(alkyl)(cycloalkyl), —N(cycloalkyl)(cycloalkyl), —R′₁₀₀, alkyl-R₁₀₀, —(CRR′)₁₋₆—P(O)(O-alkyl)₂, alkyl-O-alkyl-C(O)OH, —(CRR′)₁₋₆R′₁₀₀—(CRR′)₀₋₆R₁₀₀, —(CRR′)₁₋₆—O—R′₁₀₀, —(CRR′)₁₋₆—S—R′₁₀₀, —(CRR′)₁₋₆—C(O)—R₁₀₀, —(CRR′)₁₋₆—SO₂—R₁₀₀, and —(CRR′)₁₋₆—NR₁₀₀—R′₁₀₀ and —CH(R_{E1})—(CH₂)₀₋₃-E₁-E₂-E₃; R_{N′} is —SO₂R′₁₀₀; R and R′ are independently selected from -hydrogen, —C₁-C₁₀ alkyl (optionally independently substituted with at least one —OH), —C₁C₁₀ alkylaryl, and —C₁-C₁₀ alkylheteroaryl; R₁₀₀ and R′₁₀₀ are independently selected from -cycloalkyl, -heterocycloalkyl, -aryl, -heteroaryl, alkoxy, -aryl-W-aryl, -aryl-W-heteroaryl, -aryl-W-heterocycloalkyl, -heteroaryl-W-aryl, -heteroaryl-W-heteroaryl, -heteroaryl-W-heterocycloalkyl, -heterocycloalkyl-W-aryl, -heterocycloalkyl-W-heteroaryl, -heterocycloalkyl-W-heterocycloalkyl, -W—R₁₀₂, —CH[(CH₂)₀₋₂—O—R₁₅₀]—(CH₂)₀₋₂-aryl, —CH[(CH₂)₀₋₂—O—R₁₅₀]—(CH₂)₀₋₂-cycloalkyl, —CH[(CH₂)₀₋₂—O—R₁₅₀]—(CH₂)₀₋₂-heterocycloalkyl, —CH[(CH₂)₀₋₂—O—R₁₅₀]—(CH₂)₀₋₂-heteroaryl, —C₁-C₁₀ alkyl (optionally substituted with 1, 2, or 3 R₁₁₅ groups), wherein 1, 2, or 3 carbons of the alkyl group are optionally replaced with a group independently selected from —C(O)— and —NH—, -alkyl-O-alkyl (optionally substituted with 1, 2, or 3 R₁₁₅ groups), -alkyl-S-alkyl (optionally substituted with 1, 2, or 3 R₁₁₅ groups), and -cycloalkyl (optionally substituted with 1, 2, or 3 R₁₁₅ groups); wherein the ring portions of each group included within R₁₀₀ and R′₁₀₀ are optionally substituted with 1, 2, or 3 groups independently selected from —OR, —NO₂, -halogen, —CN, —OCF₃, —CF₃, —(CH₂)₀₋₄—O—P(═O)(OR)(OR′), —(CH₂)₀₋₄—C(O)—NR₁₀₅R′₁₀₅, —(CH₂)₀₋₄—O—(CH₂)₀₋₄—C(O)NR₁₀₂R₁₀₂′, —(CH₂)₀₋₄—C(O)—(C₁-C₁₂ alkyl), —(CH₂)₀₋₄—C(O)—(CH₂)₀₋₄-cycloalkyl —(CH₂)₀₋₄—R₁₁₀, —(CH₂)₀₋₄—R₁₂₀, —(CH₂)₀₋₄—R₁₃₀, —(CH₂)₀₋₄—C(O)—R₁₁₀, —(CH₂)₀₋₄—C(O)—R₁₂₀, —(CH₂)₀₋₄—C(O)—R₁₃₀, —(CH₂)₀₋₄—C(O)—R₁₄₀, —(CH₂)₀₋₄—C(O)—O—R₁₅₀, —(CH₂)₀₋₄—SO₂—NR₁₀₅R′₁₀₅, —(CH₂)₀₋₄—SO—(C₁-C₈ alkyl), —(CH₂)₀₋₄—SO₂—(C₁-C₁₂ alkyl, —(CH₂)₀₋₄—SO₂—(CH₂)₀₋₄-cycloalkyl, —(CH₂)₀₋₄—N(R₁₅₀)—C(O)—O—R₁₅₀, —(CH₂)₀₋₄—N(R₁₅₀)—C(O)—N(R₁₅₀)₂, —(CH₂)₀₋₄—N(R₁₅₀)—CS—N(R₁₅₀)₂, —(CH₂)₀₋₄—N(R₁₅₀)—C(O)—R₁₀₅, —(CH₂)₀₋₄—NR₁₀₅R′₁₀₅, —(CH₂)₀₋₄—R₁₄₀, —(CH₂)₀₋₄—O—C(O)-(alkyl), —(CH₂)₀₋₄—O—P(O)—(O—R₁₁₀)₂, —(CH₂)₀₋₄—O—C(O)—N(R₁₅₀)₂—(CH₂)₀₋₄—O—CS—N(R₁₅₀)₂, —(CH₂)₀₋₄—O—(R₁₅₀), —(CH₂)₀₋₄—O—R₁₅₀′—C(O)OH, —(CH₂)₀₋₄—S—(R₁₅₀), —(CH₂)₀₋₄—N(R₁₅₀)—SO₂—R₁₀₅, —(CH₂)₀₋₄-cycloalkyl, and —(C₁-C₁₀)-alkyl; R_{E1} is selected from —H, —OH, —NH₂, —NH—(CH₂)₀₋₃—R_{E2}, —NHR_{E8}, —NR_{E350}C(O)R_{E5}, —C₁-C₄ alkyl-NHC(O)R_{E5}, —(CH₂)₀₋₄R_{E8}, —O—(C₁-C₄ alkanoyl), —C₆-C₁₀ (aryloxy optionally substituted with 1, 2, or 3 groups that are independently selected from halogen, —C₁-C₄ alkyl, —CO₂H, —C(O)—C₁-C₄ alkoxy, and —C₁-C₄ alkoxy), alkoxy, -aryl-(C₁-C₄ alkoxy), —NR_{E350}CO₂R_{E351}, —C₁-C₄ alkyl-NR_{E350}CO₂R_{E351}, —CN, —CF₃, —CF₂—CF₃, —C≡CH, —CH₂—CH═CH₂, —(CH₂)₁₋₄—R_{E2}, —(CH₂)₁₋₄—NH—R_{E2}, —O—(CH₂)₀₋₃—R_{E2}, —S—(CH₂)₀₋₃—R_{E2}, —(CH₂)₀₋₄—NHC(O)—(CH₂)₀₋₆—R_{E352}, and —(CH₂)₀₋₄—(R_{E353})₀₋₁—(CH₂)₀₋₄—R_{E354}; R_{E2} is selected from —SO₂—(C₁-C₈ alkyl), —SO—(C₁-C₈ alkyl), —S—(C₁-C₈ alkyl), —S—C(O)-alkyl, —SO₂—NR_{E3}R_{E4}, —C(O)—C₁-C₂ alkyl, and —C(O)—NR_{E4}R_{E10}; R_{E3} and R_{E4} are independently selected from —H, —C₁-C₃ alkyl, and —C₃-C₆ cycloalkyl; R_{E10} is selected from alkyl, arylalkyl, alkanoyl, and arylalkanoyl; R_{E5} is selected from cycloalkyl, alkyl (optionally substituted with 1, 2, or 3 groups that are independently selected from halogen, —NR_{E6}R_{E7}, C₁-C₄ alkoxy, —C₅-C₆ heterocycloalkyl, —C₅-C₆ heteroaryl, —C₆-C₁₀ aryl, —C₃-C₇ cycloalkyl C₁-C₄ alkyl, —S—C₁-C₄ alkyl, —SO₂—C₁-C₄ alkyl, —CO₂H, —C(O)NR_{E6}R_{E7}, —CO₂—C₁-C₄ alkyl, and —C₆-C₁₀ aryloxy), heteroaryl (optionally substituted with 1, 2, or 3 groups that are independently selected from —C₁-C₄ alkyl, —C₁-C₄ alkoxy, halogen, —C₁-C₄ haloalkyl, and —OH), heterocycloalkyl (optionally substituted with 1, 2, or 3 groups independently selected from —C₁-C₄ alkyl, —C₁-C₄ alkoxy, halogen, and —C₂-C₄ alkanoyl), aryl (optionally substituted with 1, 2, 3, or 4 groups independently selected from halogen, —OH, —C₁-C₄ alkyl, —C₁-C₄ alkoxy, and —C₁-C₄ haloalkyl), and —NR_{E6}R_{E7}; R_{E6} and R_{E7} are independently selected from —H, alkyl, alkanoyl, aryl, —SO₂—C₁-C₄ alkyl, and -aryl-C₁-C₄ alkyl; R_{E8} is selected from —SO₂-heteroaryl, —SO₂-aryl, —SO₂-heterocycloalkyl, —SO₂—C₁-C₁₀ alkyl, —C(O)NHR_{E9}, heterocycloalkyl, —S-alkyl, and —S—C₂-C₄ alkanoyl; R_{E9} is selected from H, alkyl, and -aryl C₁-C₄ alkyl; R_{E350} is selected from H and alkyl; R_{E351} is selected from alkyl, -aryl-(C₁-C₄ alkyl), alkyl (optionally substituted with 1, 2, or 3 groups independently selected from halogen, cyano, heteroaryl, —NR_{E6}R_{E7}, —C(O)NR_{E6}R_{E7}, —C₃-C₇ cycloalkyl, and —C₁-C₄ alkoxy), heterocycloalkyl (optionally substituted with 1 or 2 groups independently selected from —C₁-C₄ alkyl, —C₁-C₄ alkoxy, halogen, —C₂-C₄ alkanoyl, -aryl-(C₁-C₄ alkyl), and —SO₂—(C₁-C₄ alkyl)), heteroaryl (optionally substituted with 1, 2, or 3 groups independently selected from —OH, —C₁-C₄ alkyl, —C₁-C₄ alkoxy, halogen, —NH₂, —NH(alkyl), and —N(alkyl)(alkyl)), heteroarylalkyl (optionally substituted with 1, 2, or 3 groups independently selected from —C₁-C₄ alkyl, —C₁-C₄ alkoxy, halogen, —NH₂, —NH(alkyl), and —N(alkyl)(alkyl)), aryl, heterocycloalkyl, —C₃-C₈ cycloalkyl, and cycloalkylalkyl; wherein the aryl, heterocycloalkyl, —C₃-C₈ cycloalkyl, and cycloalkylalkyl groups included within R_{E351} are optionally substituted with 1, 2, 3, 4 or 5 groups independently selected from halogen, —CN, —NO₂, alkyl, alkoxy, alkanoyl, haloalkyl, haloalkoxy, hydroxy, hydroxyalkyl, alkoxyalkyl, —C₁-C₆ thioalkoxy, —C₁-C₆ thioalkoxy-alkyl, and alkoxyalkoxy; R_{E352} is selected from heterocycloalkyl, heteroaryl, aryl, cycloalkyl, —S(O)₀₋₂-alkyl, —CO₂H, —C(O)NH₂, —C(O)NH(alkyl), —C(O)N(alkyl)(alkyl), —CO₂-alkyl, —NHS(O)₀₋₂-alkyl, —N(alkyl)S(O)₀₋₂-alkyl, —S(O)₀₋₂-heteroaryl, —S(O)₀₋₂-aryl, —NH(arylalkyl), —N(alkyl)(arylalkyl), thioalkoxy, and alkoxy; wherein each group included within R₃₅₂ is optionally substituted with 1, 2, 3, 4, or 5 groups that are independently selected from alkyl, alkoxy, thioalkoxy, halogen, haloalkyl, haloalkoxy, alkanoyl, —NO₂, —CN, alkoxycarbonyl, and aminocarbonyl; R_{E353} is selected from —O—, —C(O)—, —NH—, —N(alkyl)-, —NH—S(O)₀₋₂—, —N(alkyl)-S(O)₀₋₂—, —S(O)₀₋₂—NH—, —S(O)₀₋₂—N(alkyl)-, —NH—C(S)—, and —N(alkyl)-C(S)—; R_{E354} is selected from heteroaryl, aryl, arylalkyl, heterocycloalkyl, —CO₂H, —CO₂-alkyl, —C(O)NH(alkyl), —C(O)N(alkyl)(alkyl), —C(O)NH₂, —C₁-C₈ alkyl, —OH, aryloxy, alkoxy, arylalkoxy, —NH₂, —NH(alkyl), —N(alkyl)(alkyl), and -alkyl-CO₂-alkyl; wherein each group included within R_{E354} is optionally substituted with 1, 2, 3, 4, or 5 groups that are independently selected from alkyl, alkoxy, —CO₂H, —CO₂-alkyl, thioalkoxy, halogen, haloalkyl, haloalkoxy, hydroxyalkyl, alkanoyl, —NO₂, —CN, alkoxycarbonyl, and aminocarbonyl; E₁ is selected from —NR_{E11}— and —C₁-C₆ alkyl- (optionally substituted with 1, 2, or 3 groups selected from —C₁-C₄ alkyl), and R_{E11} is selected from —H and alkyl; or R_{E1} and R_{E11} combine to form —(CH₂)₁₋₄—; E₂ is selected from a bond, —SO₂—, —SO—, —S—, and —C(O)—; and E₃ is selected from —H, —C₁-C₄ haloalkyl, —C₅-C₆ heterocycloalkyl, —C₆-C₁₀ aryl, —OH, —N(E₃ₐ)(E_{3b}), —C₁-C₁₀ alkyl (optionally substituted with 1, 2, or 3 groups independently selected from halogen, hydroxy, alkoxy, thioalkoxy, and haloalkoxy), —C₃-C₈ cycloalkyl (optionally substituted with 1, 2, or 3 groups independently selected from —C₁-C₃ alkyl and halogen), alkoxy, aryl (optionally substituted with at least one group selected from halogen, alkyl, alkoxy, —CN and —NO₂), arylalkyl (optionally substituted with a group selected from halogen, alkyl, alkoxy, —CN, and —NO₂); —E₃ₐ and E_{3b} are independently selected from —H, —C₁-C₁₀ alkyl (optionally substituted with 1, 2, or 3 groups independently selected from halogen, —C₁-C₄ alkoxy, —C₃-C₈ cycloalkyl, and —OH), —C₂-C₆ alkyl, —C₂-C₆ alkanoyl, aryl, —SO₂-C₁-C₄ alkyl, -aryl-C₁-C₄ alkyl, and —C₃-C₈ cycloalkyl C₁-C₄ alkyl; or -E₃ₐ, E_{3b}, and the nitrogen to which they are attached may optionally form a ring selected from piperazinyl, piperidinyl, morpholinyl, and pyrrolidinyl; wherein each ring is optionally substituted with 1, 2, 3, or 4 groups that are independently selected from alkyl, alkoxy, alkoxyalkyl, and halogen; W is selected from —(CH₂)₀₋₄—, —O—, —S(O)₀₋₂—, —N(R₁₃₅)—, —CR(OH)—, and —C(O)—; R₁₀₂ and R₁₀₂′ are independently selected from hydrogen, —OH, and —C₁-C₁₀ alkyl (optionally substituted with 1, 2, or 3 groups independently selected from -halogen, -aryl, and —R₁₁₀); R₁₀₅ and R′₁₀₅ are independently selected from —H, —R₁₁₀, —R₁₂₀, -cycloalkyl, —(C₁-C₂ alkyl)-cycloalkyl, -(alkyl)-O—(C₁-C₃ alkyl), and -alkyl (optionally substituted with at least one group independently selected from —OH, -amine, or -halogen); or R₁₀₅ and R′₁₀₅ together with the atom to which they are attached form a 3, 4, 5, 6, or 7 membered carbocyclic ring, wherein one member is optionally a heteroatom selected from —O—, —S(O)₀₋₂—, and —N(R₁₃₅)—, wherein the carbocyclic ring is optionally substituted with 1, 2 or 3 R₁₄₀ groups; and wherein at least one carbon of the carbocyclic ring is optionally replaced with —C(O)—; R₁₁₀ is aryl (optionally substituted with 1 or 2 R₁₂₅ groups); R₁₁₅ at each occurrence is independently selected from halogen, —OH, —C(O)—O—R₁₀₂, —C₁-C₆ thioalkoxy, —C(O)—O-aryl, —NR₁₀₅R′₁₀₅, —SO₂—(C₁-C₈ alkyl), —C(O)—R₁₈₀, R₁₈₀, —C(O)NR₁₀₅R′₁₀₅, —SO₂NR₁₀₅R′₁₀₅, —NH—C(O)-(alkyl), —NH—C(O)—OH, —NH—C(O)—OR, —NH—C(O)—O-aryl, —O—C(O)-(alkyl), —O—C(O)-amino, —O—C(O)-monoalkylamino, —O—C(O)-dialkylamino, —O—C(O)-aryl, —O-(alkyl)-C(O)—O—H, —NH—SO₂-(alkyl), -alkoxy, and -haloalkoxy; R₁₂₀ is -heteroaryl, (optionally substituted with 1 or 2 R₁₂₅ groups); R₁₂₅ at each occurrence is independently selected from -halogen, -amino, -monoalkylamino, -dialkylamino, —OH, —CN, —SO₂—NH₂, —SO₂—NH-alkyl, —SO₂—N(alkyl)₂, —SO₂—(C₁-C₄ alkyl), —C(O)—NH₂, —C(O)—NH-alkyl, —C(O)—N(alkyl)₂, -alkyl (optionally substituted with 1, 2, or 3 groups independently selected from C₁-C₃ alkyl, halogen, —OH, —SH, —CN, —CF₃, —C₁-C₃ alkoxy, -amino, -monoalkylamino, and -dialkylamino), and -alkoxy (optionally substituted with 1, 2, or 3-halogen); R₁₃₀ is heterocycloalkyl (optionally substituted with 1 or 2 R₁₂₅ groups; R₁₃₅ is independently selected from alkyl, cycloalkyl, —(CH₂)₀₋₂-(aryl), —(CH₂)₀₋₂-(heteroaryl), and —(CH₂)₀₋₂-(heterocycloalkyl); R₁₄₀ at each occurrence is independently selected from heterocycloalkyl (optionally substituted with 1, 2, 3, or 4 groups independently selected from -alkyl, -alkoxy, -halogen, -hydroxy, -cyano, -nitro, -amino, -monoalkylamino, -dialkylamino, -haloalkyl, -haloalkoxy, -amino-alkyl, -monoalkylamino-alkyl, and -dialkylaminoalkyl); and wherein at least one carbon of the heterocycloalkyl is optionally replaced with —C(O); R₁₅₀ is independently selected from -hydrogen, -cycloalkyl, —(C₁-C₂ alkyl)-cycloalkyl, —R₁₁₀, —R₁₂₀, and -alkyl (optionally substituted with 1, 2, 3, or 4 groups independently selected from —OH, —NH₂, —C₁-C₃ alkoxy, —R₁₁₀, and -halogen); R₁₅₀′ is independently selected from -cycloalkyl, —(C₁-C₃ alkyl)-cycloalkyl, —R₁₁₀, —R₁₂₀, and -alkyl (optionally substituted with 1, 2, 3, or 4 groups independently selected from —OH, —NH₂, —C₁-C₃ alkoxy, —R₁₁₀, and -halogen); and R₁₈₀ is independently selected from -morpholinyl, -thiomorpholinyl, -piperazinyl, -piperidinyl, -homomorpholinyl, -homothiomorpholinyl, -homothiomorpholinyl S-oxide, -homothiomorpholinyl S,S-dioxide, -pyrrolinyl, and -pyrrolidinyl; wherein each R₁₈₀ is optionally substituted with 1, 2, 3, or 4 groups independently selected from -alkyl, -alkoxy, -halogen, -hydroxy, -cyano, -nitro, -amino, -monoalkylamino, -dialkylamino, -haloalkyl, -haloalkoxy, -aminoalkyl, -monoalkylamino-alkyl, -dialkylamino-alkyl, and —C(O); and wherein at least one carbon of R₁₈₀ is optionally replaced with —C(O)—; R_{C} is selected from fused rings of formulae (IIIa) and (IIIb), 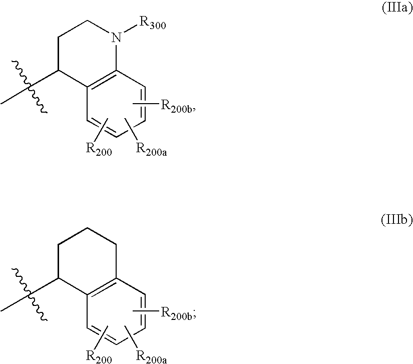
wherein 1, 2, or 3 carbons of the cycloalkyl of formulae (IIIa) and (IIIb) are optionally replaced with —C(O)—, —O—, and —S(O)₂—, wherein at least one carbon of the fused heterocycloalkyl of IIIa and at least one carbon of the cycloalkyl of IIIb is optionally substituted with one or two groups each independently selected from —R₂₀₅, —R₂₄₅, and —R₂₅₀; R₂₀₀, R₂₀₀ₐ, and R_{200b} at each occurrence are independently selected from —H, -alkyl (optionally substituted with at least one group independently selected from R₂₀₅), —OH, —NO₂, -halogen, —CN, —(CH₂)₀₋₄—C(O)H, —(CO)₀₋₁R₂₁₅, —(CO)₀₋₁R₂₂₀, —(CH₂)₀₋₄—(CO)₀₋₁—NR₂₂₀R₂₂₅, —(CH₂)₀₋₄—C(O)-alkyl, —(CH₂)₀₋₄—(CO)₀₋₁-cycloalkyl, —(CH₂)₀₋₄—(CO)₀₋₁-heterocycloalkyl, —(CH₂)₀₋₄—(CO)₀₋₁-aryl, —(CH₂)₀₋₄—(CO)₀₋₁-heteroaryl, —(CH₂)₀₋₄—CO₂R₂₁₅, —(CH₂)₀₋₄—SO₂—NR₂₂₀R₂₂₅, —(CH₂)₀₋₄—S(O)₀₋₂-alkyl, —(CH₂)₀₋₄—S(O)₀₋₂-cycloalkyl, —(CH₂)₀₋₄—N(H or R₂₁₅)—CO₂R₂₁₅, —(CH₂)₀₋₄—N(H or R₂₁₅)—SO₂—R₂₂₀, —(CH₂)₀₋₄—N(H or R₂₁₅)—C(O)—N(R₂₁₅)₂, —(CH₂)₀₋₄—N(H or R₂₁₅)—C(O)—R₂₂₀, —(CH₂)₀₋₄—O—C(O)-alkyl, —(CH₂)₀₋₄—O—(R₂₁₅), —(CH₂)₀₋₄—S—(R₂₁₅), —(CH₂)₀₋₄—O-alkyl (optionally substituted with at least one halogen), and -adamantane; wherein each aryl and heteroaryl group included within R₂₀₀ is optionally substituted with at least one group independently selected from —R₂₀₅, —R₂₁₀, and -alkyl (optionally substituted with at least one group independently selected from R₂₀₅ and R₂₁₀); wherein each cycloalkyl or heterocycloalkyl group included within R₂₀₀ is optionally substituted with at least one group independently selected from R₂₁₀; R₂₀₅ at each occurrence is independently selected from -alkyl, -haloalkoxy, —(CH₂)₀₋₃-cycloalkyl, -halogen, —(CH₂)₀₋₆—OH, -aryl, —O-aryl, —OH, —SH, —(CH₂)₀₋₄—C(O)H, —(CH₂)₀₋₆—CN, —(CH₂)₀₋₆—C(O)—NR₂₃₅R₂₄₀, —(CH₂)₀₋₆—C(O)—R₂₃₅, —(CH₂)₀₋₄—N(H or R₂₁₅)—SO₂—R₂₃₅, —CF₃, —CN, -alkoxy, -alkoxycarbonyl, and —NR₂₃₅R₂₄₀; R₂₁₀ at each occurrence is independently selected from —OH, —CN, —(CH₂)₀₋₄—C(O)H, -alkyl (optionally substituted with at least one group independently selected from R₂₀₅), —S(O)₂-alkyl, -halogen, -alkoxy, -haloalkoxy, —NR₂₂₀R₂₂₅, -cycloalkyl (optionally substituted with at least one group independently selected from R₂₀₅), —C(O)-alkyl, —S(O)₂—NR₂₃₅R₂₄₀, —C(O)—NR₂₃₅R₂₄₀, and —S-alkyl; R₂₁₅ at each occurrence is independently selected from -alkyl, —(CH₂)₀₋₂-cycloalkyl, —(CH₂)₀₋₂-aryl, —(CH₂)₀₋₂-heteroaryl, —(CH₂)₀₋₂-heterocycloalkyl, and —CO₂—CH₂-aryl; wherein the aryl groups included within R₂₁₅ are optionally substituted with at least one group independently selected from R₂₀₅ and R₂₁₀, and wherein the heterocycloalkyl and heteroaryl groups included within R₂₁₅ are optionally substituted with R₂₁₀; R₂₂₀ and R₂₂₅ at each occurrence are independently selected from —H, -alkyl, —(CH₂)₀₋₄—C(O)H, —(CH₂)₀₋₄—C(O)-alkyl, -alkylhydroxy, -alkoxycarbonyl, -alkylamino, —S(O)₂-alkyl, —C(O)-alkyl (optionally substituted with at least one halogen), —C(O)—NH₂, —C(O)—NH(alkyl), —C(O)—N(alkyl)(alkyl), -haloalkyl, —(CH₂)₀₋₂-cycloalkyl, -(alkyl)-O-(alkyl), -aryl, -heteroaryl, and -heterocycloalkyl; wherein the aryl, heteroaryl and heterocycloalkyl groups included within R₂₂₀ and R₂₂₅ are each optionally substituted with at least one group independently selected from R₂₇₀; R₂₃₅ and R₂₄₀ at each occurrence are independently selected from —H, —OH, —CF₃, —OCH₃, —NH—CH₃, —N(CH₃)₂, —(CH₂)₀₋₄—C(O)—(H or alkyl), -alkyl, —C(O)-alkyl, —SO₂-alkyl, and -aryl; R₂₄₅ and R₂₅₀ at each occurrence are independently selected from —H, —OH, —(CH₂)₀₋₄CO₂-alkyl, —(CH₂)₀₋₄C(O)-alkyl, -alkyl, -hydroxyalkyl, -alkoxy, -haloalkoxy, —(CH₂)₀₋₄-cycloalkyl, —(CH₂)₀₋₄-aryl, —(CH₂)₀₋₄-heteroaryl, and —(CH₂)₀₋₄-heterocycloalkyl; or R₂₄₅ and R₂₅₀ are taken together with the carbon to which they are attached to form a monocyclic or bicyclic ring system of 3-8 carbon atoms, wherein at least one carbon atom of the monocyclic or bicyclic ring system is optionally replaced by at least one group independently selected from —O—, —S—, —SO₂—, —C(O)—, —NR₂₂₀—, and —N(alkyl)(alkyl); and wherein the ring is optionally substituted with at least one group independently selected from -alkyl, -alkoxy, —OH, —NH₂, —NH(alkyl), —N(alkyl)(alkyl), —NH—C(O)-alkyl, —NH—SO₂-alkyl, and halogen; wherein the aryl, heteroaryl, or heterocycloalkyl groups included within R₂₄₅ and R₂₅₀ are optionally substituted with at least one group independently selected from halogen, alkyl, —CN, and —OH; R₂₇₀ at each occurrence is independently selected from —R₂₀₅, -alkyl (optionally substituted with at least one group independently selected from R₂₀₅), -aryl, -halogen, -alkoxy, -haloalkoxy, —NR₂₃₅R₂₄₀, —OH, —CN, -cycloalkyl (optionally substituted with at least one group independently selected from R₂₀₅), —C(O)-alkyl, —S(O)₂—NR₂₃₅R₂₄₀, —CO—NR₂₃₅R₂₄₀, —S(O)₂-alkyl, and —(CH₂)₀₋₄—C(O)H; R₃₀₀ is selected from —H, —(CO)₀₋₁R₂₁₅, and —(CO)₀₋₁R₂₂₀; wherein at least one carbon of the aryl group of formulae (IIIa) or (IIIb) is optionally replaced by a heteroatom.

An embodiment of the present invention is to provide selective compounds of formula (I), 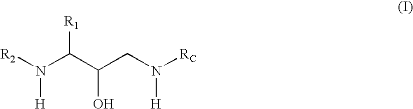
or a pharmaceutically acceptable salt thereof, wherein R₁, R₂, and R_{C} are defined above.

Another embodiment of the present invention is to provide efficacious compounds of formula (I), or a pharmaceutically acceptable salt thereof, wherein the inhibition is at least 10% for a dose of about 100 mg/kg or less, and wherein R₁, R₂, and R_{C} are defined above.

Another embodiment of the present invention is to provide orally bioavailable compounds of formula (I), or a pharmaceutically acceptable salt thereof, wherein the compound has an F value of at least 10%, and wherein R₁, R₂, and R_{C} are defined above.

In an embodiment, the present invention provides a method of preventing or treating conditions which benefit from inhibition of at least one aspartyl-protease, comprising administering to a host a composition comprising a therapeutically effective amount of at least one compound of the formula, 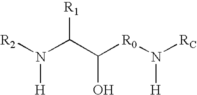
or a pharmaceutically acceptable salt thereof, wherein R₁, R₂, and R_{C} are as defined below and R₀ is selected from —CH(alkyl), —C(alky)₂-, —CH(cycloalkyl)-, —C(alkyl)(cycloalkyl)-, and —C(cycloalkyl)₂.

In an embodiment, the hydroxyl alpha to the —(CHR₁)— group of formula (I) may be optionally replaced by —NH₂, —NHR₇₀₀, —NR₇₀₀R₇₀₀, —SH, and —SR₇₀₀, wherein R₇₀₀ is alkyl (optionally substituted with at least one group independently selected from R₁₁₀, R₁₁₅, R₂₀₅, and R₂₁₀); wherein R₁₁₀, R₁₁₅, R₂₀₅, and R₂₁₀ are defined above.

In another embodiment, R₁ is selected from —CH₂-phenyl, wherein the phenyl ring is optionally substituted with at least one group independently selected from halogen, alkyl, alkoxy, and —OH.

In another embodiment, R₁ is selected from 3-Allyloxy-5-fluoro-benzyl, 3-Benzyloxy-5-fluoro-benzyl, 4-hydroxy-benzyl, 3-hydroxy-benzyl, 3-propyl-thiophen-2-yl-methyl, 3,5-difluoro-2-propylamino-benzyl, 5-chloro-thiophen-2-yl-methyl, 5-chloro-3-ethyl-thiophen-2-yl-methyl, 3,5-difluoro-2-hydroxy-benzyl, 2-ethylamino-3,5-difluoro-benzyl, piperidin-4-yl-methyl, 2-oxo-piperidin-4-yl-methyl, 2-oxo-1,2-dihydro-pyridin-4-yl-methyl, 5-hydroxy-6-oxo-6H-pyran-2-yl-methyl, 2-Hydroxy-5-methyl-benzamide, 3,5-Difluoro-4-hydroxy-benzyl, 3,5-Difluoro-benzyl, 3-Fluoro-4-hydroxy-benzyl, 3-Fluoro-5-[2-(2-methoxy-ethoxy)-ethoxy]-benzyl, 3-Fluoro-5-heptyloxy-benzyl, 3-Fluoro-5-hexyloxy-benzyl, 3-Fluoro-5-hydroxy-benzyl, and 3-Fluoro-benzyl.

In another embodiment, R₂ is selected from —C(O)—CH₃ and —C(O)—CH₂F.

In another embodiment, R₂ is selected from tert-butyl formate, 2,2-difluoroacetaldehyde, 2-hydroxyacetaldehyde, hydrosulfonylmethane, N-(3-formylphenyl)methanesulfonamide, and N-(3-formylphenyl)-N-methylmethanesulfonamide,

In another embodiment, R₂ is selected from glyoxylic acid, crotonic acid, pyruvic acid, butyric acid, sarcosine, 3-hydroxy-propionic acid, methoxyacetic acid, chloroacetic acid, penta-2,4-dienoic acid, pent-4-ynoic acid, 1-methyl-cyclopropanecarboxylic acid, pent-4-enoic acid, cyclopropylacetic acid, cyclobutanecarboxylic acid, trans-2-pentenoic acid, valeric acid, DL-2-ethylpropionic acid, isovaleric acid, 2-hydroxy-2-methyl-propionic acid, ethoxyacetic acid, DL-2-hydroxy-n-butyric acid, furan-3-carboxylic acid, 1H-pyrazole-4-carboxylic acid, 1H-imidazole-4-carboxylic acid, cyclopent-1-enecarboxylic acid, 4-Methyl-pent-2-enoic acid, cyclopentanecarboxylic acid, trans-2-hexenoic acid, 2-oxo-pentanoic acid, levulinic acid, tetrahydro-3-fluroic acid, tetrahydrofuran-2-carboxylic acid, caproic acid, tert-butylacetic acid, methylmalonic acid, 2-hydroxy-3-methyl-butyric acid, benzoic acid, 2-chloro-butyric acid, picolonic acid, nicotinic acid, isonicotinic acid, pyrazine-2-carboxylic acid, 3-methyl-furan-2-carboxylic acid, 1-methyl-1H-pyrazole-3-carboxylic acid, cyclopent-2-enyl-acetic acid, 5-methyl-isoxazole-3-carboxylic acid, thiophene-3-carboxylic acid, 2-Methyl-hex-2-enoic acid, L-pyroglutamic acid, 5-oxo-pyrrolidine-2-carboxylic acid, D-pyroglutamic acid, N-methylaleamic acid, thiazole 5-carboxylic acid, N-Me-Pro-OH, 3-Methyl-pyrrolidine-2-carboxylic acid, itaconic acid, citraconic acid, 2-oxo-imidazolidine-4-carboxylic acid, 4-Methyl-2-oxo-pentanoic acid, enanthic acid, L-hydroxyproline, Cis-4-hydroxy-D-proline, 6-Amino-hexanoic acid, oxalacetic acid, Mono-methyl succinate, Butoxy-acetic acid, (S)-(−)-2-hydroxy-3,3-dimethylbutyric acid, (2-methoxy-ethoxy)-acetic acid, Phenylacetic acid, 5-Chloro-pentanoic acid, Anthranilic acid, Aminonicotinic acid, 3-Hydroxy-pyridine-2-carboxylic acid, 2-Hydroxy-nicotinic acid, Furan-2-yl-oxo-acetic acid, 5-Formyl-furan-2-carboxylic acid, 6-Hydroxy-pyrimidine-4-carboxylic acid, 3-Furan-2-yl-propionic acid, Norbornane-2-carboxylic acid, 1-cyclohexenylacetic acid, 3,5-Dimethyl-isoxazole-4-carboxylic acid, Hexa-2,4-dienedioic acid, (2-Oxo-cyclopentyl)-acetic acid, 5-Methyl-thiophene-2-carboxylic acid, Thiophene-2-acetic acid, cylcohexylacetic acid, methyl cyclohexanone-2-carboxylate, (2-Imino-imidazolidin-1-yl)-acetic acid, 4-amino-cyclohexanecarboxylic acid, 2-methylene-succinic acid 1-methyl ester, Trans-beta-hydromuconic acid, Octanoic acid, 2-Propyl-pentanoic acid, 4-Acetylamino-butyric acid, 2-Oxo-pentanedioic acid, N-carbamyl-alpha-aminoisobutyric acid, 4-cyano-benzoic acid, and 2-Acetylamino-3-hydroxy-propionic acid.

In another embodiment, U is selected from —C(O)—, —C(S)—, —S(O)₀₋₂—, —C(NR₂₁)—, —C(N—OR₂₁)—, —C(O)—NR₂₀—, —C(O)—O—, —S(O)₂—NR₂₀—, and —S(O)₂—O—; and V is —(T)₀₋₁—R_{N}.

In another embodiment, U is —C(O)—.

In another embodiment, U is selected from —C(O)— and —S(O)₀₋₂—; and V is selected from alkyl, alkoxy, aryl, heteroaryl, cycloalkyl, and heterocycloalkyl; wherein the alkyl included within V are optionally substituted with at least one group independently selected from —OH, —NH₂, and halogen; and wherein the aryl, heteroaryl, cycloalkyl, and heterocycloalkyl groups included within V are optionally substituted with 1 or 2 R_{B} groups.

In another embodiment, U′ is selected from —C(O)—, —C(NR₂₁)—, —C(N—OR₂₁)—, —C(O)—NR₂₀—, and —C(O)—O—; and V′ is —(T)₀₋₁—R_{N′}.

In another embodiment, R_{N} is selected from alkyl, —(CH₂)₀₋₂-aryl, C₂-C₆ alkyl, C₃-C₇ cycloalkyl, —(CH₂)₀₋₂-heteroaryl, and 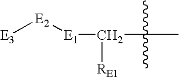
wherein E₁ is selected from —NR_{E11}— and C₁-C₆ alkyl optionally substituted with 1, 2, or 3 C₁-C₄ groups, R_{E1} is —NH₂, and R_{E11} is selected from —H and alkyl, or R_{E1} and R_{E11} combine to form —(CH₂)₁₋₄—; E₂ is selected from a bond; SO₂, SO, S, and C(O); E₃ is selected from —H, —C₁-C₄ haloalkyl, —C₅-C₆ heterocycloalkyl containing at least one N, O, or S, -aryl, —OH, —N(E₃ₐ)(E_{3b}), —C₁-C₁₀ alkyl optionally substituted with 1, 2, or thru 3 groups which can be the same independently or different and are se selected from halogen, hydroxy, alkoxy, thioalkoxy, and haloalkoxy, —C₃-C₈ cycloalkyl optionally substituted with 1, 2, or 3 groups independently selected from C₁-C₃ alkyl, and halogen, -alkoxy, -aryl optionally substituted with at least one group selected from halogen, C₁-C₄ alkyl, C₁-C₄ alkoxy, —CN, and —NO₂ and -aryl C₁-C₄ alkyl optionally substituted with at least one group selected from halogen, C₁-C₄ alkyl, C₁-C₄ alkoxy, —CN, and —NO₂, E₃ₐ and E_{3b} are independently selected from —H, —C₁-C₁₀ alkyl optionally substituted with 1, 2, or 3 groups independently selected from halogen, C₁-C₄ alkoxy, C₃-C₈ cycloalkyl, and —OH, —C₂-C₆ alkanoyl, -aryl, —SO₂—C₁-C₄ alkyl, -aryl C₁-C₄ alkyl, and —C₃-C₈ cycloalkyl C₁-C₄ alkyl, or E₃ₐ, E_{3b}, and the nitrogen to which they are attached form a ring selected from piperazinyl, piperidinyl, morpholinyl, and pyrolidinyl, wherein each ring is optionally substituted with 1, 2, 3, or 4 groups that are independently selected from alkyl, alkoxy, alkoxyalkyl, and halogen.

In another embodiment, V is —(CH₂)₁₋₃-aryl or —(CH₂)₁₋₃-heteroaryl, wherein each ring is independently optionally substituted with 1 or 2 groups independently selected from halogen, —OH, —OCF₃, —O-aryl, —CN, —NR₁₀₁R′₁₀₁, alkyl, alkoxy, (CH₂)₀₋₃(C₃-C₇ cycloalkyl), aryl, heteroaryl, and heterocycloalkyl, and wherein the alkyl, alkoxy, cycloalkyl, aryl, heteroaryl, or heterocycloalkyl groups are optionally substituted with 1 or 2 groups independently selected from C₁-C₄ alkyl, C₁-C₄ alkoxy, C₁-C₄ haloalkyl, C₁-C₄ haloalkoxy, halogen, —OH, —CN, and —NR₁₀₁R′₁₀₁.

In another embodiment, R_{C} is selected from 7-(4-methyl-thiophen-3-yl)-1,2,3,4-tetrahydro-naphthalen-1-yl, 7-(3-methyl-3h-imidazol-4-yl)-1,2,3,4-tetrahydro-naphthalen-1-yl, 7-(4-methyl-pyridin-2-yl)-1,2,3,4-tetrahydro-naphthalen-1-yl, 7-pyrimidin-2-yl-1,2,3,4-tetrahydro-naphthalen-1-yl, 7-isopropenyl-1,2,3,4-tetrahydro-naphthalen-1-yl, 7-(4-trifluoromethyl-pyrimidin-2-yl)-1,2,3,4-tetrahydro-naphthalen-1-yl, 7-(2-methylsulfanyl-pyrimidin-4-yl)-1,2,3,4-tetrahydro-naphthalen-1-yl, 7-pyrimidin-5-yl-1,2,3,4-tetrahydro-naphthalen-1-yl, 7-pyridin-2-yl-1,2,3,4-tetrahydro-naphthalen-1-yl, 7-(5-methyl-pyridin-2-yl)-1,2,3,4-tetrahydro-naphthalen-1-yl 7-pyridin-3-yl-1,2,3,4-tetrahydro-naphthalen-1-yl, 7-(3-methyl-pyridin-2-yl)-1,2,3,4-tetrahydro-naphthalen-1-yl, 7-(6-methyl-pyridazin-3-yl)-1,2,3,4-tetrahydro-naphthalen-1-yl, 7-pyridin-4-yl-1,2,3,4-tetrahydro-naphthalen-1-yl, 7-(6-methyl-pyridin-3-yl)-1,2,3,4-tetrahydro-naphthalen-1-yl, 7-(6-methoxy-pyridazin-3-yl)-1,2,3,4-tetrahydro-naphthalen-1-yl, 7-(4-methyl-pyridin-3-yl)-1,2,3,4-tetrahydro-naphthalen-1-yl, 7-pyrazin-2-yl-1,2,3,4-tetrahydro-naphthalen-1-yl, 7-(5-methyl-thiophen-2-yl)-1,2,3,4-tetrahydro-naphthalen-1-yl, 7-thiazol-2-yl-1,2,3,4-tetrahydro-naphthalen-1-yl, 7-thiophen-3-yl-1,2,3,4-tetrahydro-naphthalen-1-yl, 7-(1-methyl-1h-imidazol-2-yl)-1,2,3,4-tetrahydro-naphthalen-1-yl, 7-thiophen-2-yl-1,2,3,4-tetrahydro-naphthalen-1-yl, 7-(3-methyl-thiophen-2-yl)-1,2,3,4-tetrahydro-naphthalen-1-yl, 5-(3-amino-phenyl)-7-ethyl-1,2,3,4-tetrahydro-naphthalen-1-yl, 7-ethyl-5-thiazol-2-yl-1,2,3,4-tetrahydro-naphthalen-1-yl, 7-ethyl-5-pyridin-2-yl-1,2,3,4-tetrahydro-naphthalen-1-yl, 7-ethyl-5-(3-methyl-pyridin-2-yl)-1,2,3,4-tetrahydro-naphthalen-1-yl, 7-ethyl-5-(4-methyl-pyridin-2-yl)-1,2,3,4-tetrahydro-naphthalen-1-yl, 7-(2,2-dimethyl-propyl)-1,2,3,4-tetrahydro-naphthalen-1-yl, 6-(2,2-dimethyl-propyl)-1-methyl-1,2,3,4-tetrahydro-quinolin-4-yl, 7-(2,2-dimethyl-propyl)-4-oxo-1,2,3,4-tetrahydro-naphthalen-1-yl, 7-(2,2-dimethyl-propyl)-5-ethyl-1,2,3,4-tetrahydro-naphthalen-1-yl, 6-(2,2-dimethyl-propyl)-1,2,3,4-tetrahydro-quinolin-4-yl, 7-(2,2-dimethyl-propyl)-1-methyl-1,2,3,4-tetrahydro-naphthalen-1-yl, 7-propyl-1,2,3,4-tetrahydro-naphthalen-1-yl, 6-isopropyl-2-oxo-1,2,3,4-tetrahydro-quinolin-4-yl, 7-isopropyl-3-oxo-1,2,3,4-tetrahydro-naphthalen-1-yl, 3-hydroxy-7-isopropyl-3-methyl-1,2,3,4-tetrahydro-naphthalen-1-yl, 3-acetylamino-7-isopropyl-1,2,3,4-tetrahydro-naphthalen-1-yl, 7-isopropyl-3-methanesulfonylamino-1,2,3,4-tetrahydro-naphthalen-1-yl, 1,2,3,4-tetrahydro-naphthalen-1-yl, 7-methoxy-1,2,3,4-tetrahydro-naphthalen-1-yl, 7-ethyl-1,2,3,4-tetrahydro-naphthalen-1-yl, 6-ethyl-1-methyl-1,2,3,4-tetrahydro-quinolin-4-yl, 7-dimethylaminomethyl-1,2,3,4-tetrahydro-naphthalen-1-yl, 7-bromo-1,2,3,4-tetrahydro-naphthalen-1-yl, 6-carbobenzoxy-1,2,3,4-tetrahydro-quinolin-4-yl, 7-ethyl-2,2-dimethyl-1,2,3,4-tetrahydro-naphthalen-1-yl, 7-isobutyl-1,2,3,4-tetrahydro-naphthalen-1-yl, 5-bromo-7-ethyl-1,2,3,4-tetrahydro-naphthalen-1-yl, 5,7-diethyl-1,2,3,4-tetrahydro-naphthalen-1-yl, 5-butyl-7-ethyl-1,2,3,4-tetrahydro-naphthalen-1-yl, 7-ethyl-5-propyl-1,2,3,4-tetrahydro-naphthalen-1-yl, 7-ethyl-5-isobutyl-1,2,3,4-tetrahydro-naphthalen-1-yl, 7-(2,2-dimethyl-propyl)-2-hydroxymethyl-1,2,3,4-tetrahydro-naphthalen-1-yl, 7-ethyl-5-(5-methyl-pyridin-2-yl)-1,2,3,4-tetrahydro-naphthalen-1-yl, 7-ethyl-5-(6-methyl-pyridin-2-yl)-1,2,3,4-tetrahydro-naphthalen-1-yl, 7-butyl-1,2,3,4-tetrahydro-naphthalen-1-yl, 5-cyano-7-ethyl-1,2,3,4-tetrahydro-naphthalen-1-yl, 6-ethyl-1,2,3,4-tetrahydro-quinolin-4-yl, 7-ethyl-1-methyl-1,2,3,4-tetrahydro-naphthalen-1-yl, 7-sec-butyl-1,2,3,4-tetrahydro-naphthalen-1-yl, 2-hydroxy-6-neopentyl-1,2,3,4-tetrahydroquinolin-4-yl, 2-hydroxy-6-isobutyl-1,2,3,4-tetrahydroquinolin-4-yl, 2-hydroxy-6-isopropyl-1,2,3,4-tetrahydroquinolin-4-yl, 6-tert-butyl-1,2,3,4-tetrahydroquinolin-4-yl, 6-ethyl-1,2,3,4-tetrahydroquinolin-4-yl, 7-fluoro-6-isopropyl-1,2,3,4-tetrahydroquinolin-4-yl, 6-tert-butyl-7-fluoro-1,2,3,4-tetrahydroquinolin-4-yl, 7-fluoro-6-isobutyl-1,2,3,4-tetrahydroquinolin-4-yl, 7-fluoro-6-neopentyl-1,2,3,4-tetrahydroquinolin-4-yl, 2-hydroxy-1-methyl-6-neopentyl-1,2,3,4-tetrahydroquinolin-4-yl, 2-hydroxy-6-isobutyl-1-methyl-1,2,3,4-tetrahydroquinolin-4-yl, 2-hydroxy-6-isopropyl-1-methyl-1,2,3,4-tetrahydroquinolin-4-yl, 6-tert-butyl-1-methyl-1,2,3,4-tetrahydroquinolin-4-yl, 6-tert-butyl-1-(2-hydroxyethyl)-1,2,3,4-tetrahydroquinolin-4-yl, 2-hydroxy-1-(2-hydroxyethyl)-6-isopropyl-1,2,3,4-tetrahydroquinolin-4-yl, 2-hydroxy-1-(2-hydroxyethyl)-6-isobutyl-1,2,3,4-tetrahydroquinolin-4-yl, 2-hydroxy-1-(2-hydroxyethyl)-6-neopentyl-1,2,3,4-tetrahydroquinolin-4-yl, 1-acetyl-6-neopentyl-1,2,3,4-tetrahydroquinolin-4-yl, 1-acetyl-6-isobutyl-1,2,3,4-tetrahydroquinolin-4-yl, 1-acetyl-6-isopropyl-1,2,3,4-tetrahydroquinolin-4-yl, 1-acetyl-6-tert-butyl-1,2,3,4-tetrahydroquinolin-4-yl, 6-tert-butyl-1-(cyanomethyl)-1,2,3,4-tetrahydroquinolin-4-yl, 1-(cyanomethyl)-6-isopropyl-1,2,3,4-tetrahydroquinolin-4-yl, 1-(cyanomethyl)-6-isobutyl-1,2,3,4-tetrahydroquinolin-4-yl, 1-(cyanomethyl)-6-neopentyl-1,2,3,4-tetrahydroquinolin-4-yl, 2-hydroxy-6-(1-hydroxy-2,2-dimethylpropyl)-1,2,3,4-tetrahydroquinolin-4-yl, 2-hydroxy-6-(1-hydroxy-2,2-dimethylpropyl)-1-methyl-1,2,3,4-tetrahydroquinolin-4-yl, 2,2-dimethyl-6-neopentyl-1,2,3,4-tetrahydroquinolin-4-yl, 2-hydroxy-1,2,2-trimethyl-6-neopentyl-1,2,3,4-tetrahydroquinolin-4-yl, 1,4-dimethyl-6-neopentyl-1,2,3,4-tetrahydroquinolin-4-yl, 2-hydroxy-4-methyl-6-neopentyl-1,2,3,4-tetrahydroquinolin-4-yl, 2-hydroxy-6-isobutyl-4-methyl-1,2,3,4-tetrahydroquinolin-4-yl, 2-hydroxy-6-isobutyl-1,4-dimethyl-1,2,3,4-tetrahydroquinolin-4-yl, 6-tert-butoxy-1,2,3,4-tetrahydroquinolin-4-yl, 6-tert-butoxy-4-methyl-1,2,3,4-tetrahydroquinolin-4-yl, 6-tert-butoxy-4,8-dimethyl-1,2,3,4-tetrahydroquinolin-4-yl, 2-hydroxy-4-methyl-6-neopentyl-1,2,3,4-tetrahydroquinolin-4-yl, 4,8-dimethyl-6-neopentyl-1,2,3,4-tetrahydroquinolin-4-yl, 2-hydroxy-8-methyl-6-neopentyl-1,2,3,4-tetrahydroquinolin-4-yl, 2-hydroxy-6-(2-hydroxy-2-methylpropyl)-8-methyl-1,2,3,4-tetrahydroquinolin-4-yl, 2-hydroxy-6-(2-hydroxy-2-methylpropyl)-4-methyl-1,2,3,4-tetrahydroquinolin-4-yl, 2-hydroxy-6-(2-hydroxy-2-methylpropyl)-1,2,3,4-tetrahydroquinolin-4-yl, 2-hydroxy-6-(1-hydroxy-2,2-dimethylpropyl)-1,2,3,4-tetrahydroquinolin-4-yl, 2-hydroxy-6-(1-hydroxy-2,2-dimethylpropyl)-4-methyl-1,2,3,4-tetrahydroquinolin-4-yl, 2-hydroxy-5-isobutyl-2-pyridin-3-ylbenzyl, 2-hydroxy-5-isobutyl-2-pyridin-4-ylbenzyl, 2-hydroxy-5-isobutyl-2-(6-methoxypyridin-3-yl)benzyl, 2-hydroxy-5-isobutyl-2-(5-methoxypyridin-3-yl)benzyl, 5,7-diethyl-1,2,3,4-tetrahydronaphthalen-1-yl, 7-ethyl-5-propyl-1,2,3,4-tetrahydro-naphthalen-1-yl, 7-ethyl-5-isobutyl-1,2,3,4-tetrahydronaphthalen-1-yl, 7-(3,6-dimethyl-pyrazin-2-yl)-1,2,3,4-tetrahydro-naphthalen-1-yl, 7-furan-2-yl-1,2,3,4-tetrahydro-naphthalen-1-yl, 7-styryl-1,2,3,4-tetrahydro-naphthalen-1-yl, 7-(3,5-dimethyl-isoxazol-4-yl)-1,2,3,4-tetrahydro-naphthalen-1-yl, 7-(5-ethyl-pyrimidin-2-yl)-1,2,3,4-tetrahydro-naphthalen-1-yl, 1-[3-(5-acetyl-thiophen-2-yl)-phenyl]-cyclopropyl, 1-(3-thiophen-3-yl-phenyl)-cyclopropyl, 1-[3-(6-methoxy-pyridin-3-yl)-phenyl]-cyclopropyl, 1-(3-furan-3-yl-phenyl)-cyclopropyl, 1-[3-(3,5-dimethyl-isoxazol-4-yl)-phenyl]-cyclopropyl, and 5-(3-aminophenyl)-7-ethyl-1,2,3,4-tetrahydronaphthalen-1-yl, or a pharmaceutically acceptable salt thereof.

In another embodiment, the at least one compound of formula (I) is N-{1-(3,5-Difluoro-benzyl)-2-hydroxy-3-[7-(4-methyl-thiophen-3-yl)-1,2,3,4-tetrahydro-naphthalen-1-ylamino]-propyl}-acetamide, N-{1-(3,5-Difluoro-benzyl)-2-hydroxy-3-[7-(3-methyl-3H-imidazol-4-yl)-1,2,3,4-tetrahydro-naphthalen-1-ylamino]-propyl}-acetamide, N-{1-(3,5-Difluoro-benzyl)-2-hydroxy-3-[7-(4-methyl-pyridin-2-yl)-1,2,3,4-tetrahydro-naphthalen-1-ylamino]-propyl}-acetamide, N-[1-(3,5-Difluoro-benzyl)-2-hydroxy-3-(7-pyrimidin-2-yl-1,2,3,4-tetrahydro-naphthalen-1-ylamino)-propyl]-acetamide, N-[1-(3,5-Difluoro-benzyl)-2-hydroxy-3-(7-isopropenyl-1,2,3,4-tetrahydro-naphthalen-1-ylamino)-propyl]-acetamide, N-{1-(3,5-Difluoro-benzyl)-2-hydroxy-3-[7-(4-trifluoromethyl-pyrimidin-2-yl)-1,2,3,4-tetrahydro-naphthalen-1-ylamino]-propyl}-acetamide, N-{1-(3,5-Difluoro-benzyl)-2-hydroxy-3-[7-(2-methylsulfanyl-pyrimidin-4-yl)-1,2,3,4-tetrahydro-naphthalen-1-ylamino]-propyl}-acetamide, N-[1-(3,5-Difluoro-benzyl)-2-hydroxy-3-(7-pyrimidin-5-yl-1,2,3,4-tetrahydro-naphthalen-1-ylamino)-propyl]-acetamide, N-[1-(3,5-Difluoro-benzyl)-2-hydroxy-3-(7-pyridin-2-yl-1,2,3,4-tetrahydro-naphthalen-1-ylamino)-propyl]-acetamide, N-{1-(3,5-Difluoro-benzyl)-2-hydroxy-3-[7-(5-methyl-pyridin-2-yl)-1,2,3,4-tetrahydro-naphthalen-1-ylamino]-propyl}-acetamide, N-[1-(3,5-Difluoro-benzyl)-2-hydroxy-3-(7-pyridin-3-yl-1,2,3,4-tetrahydro-naphthalen-1-ylamino)-propyl]-acetamide, N-{1-(3,5-Difluoro-benzyl)-2-hydroxy-3-[7-(3-methyl-pyridin-2-yl)-1,2,3,4-tetrahydro-naphthalen-1-ylamino]-propyl}-acetamide, N-{1-(3,5-Difluoro-benzyl)-2-hydroxy-3-[7-(6-methyl-pyridazin-3-yl)-1,2,3,4-tetrahydro-naphthalen-1-ylamino]-propyl}-acetamide, N-[1-(3,5-Difluoro-benzyl)-2-hydroxy-3-(7-pyridin-4-yl-1,2,3,4-tetrahydro-naphthalen-1-ylamino)-propyl]-acetamide, N-{1-(3,5-Difluoro-benzyl)-2-hydroxy-3-[7-(6-methyl-pyridin-3-yl)-1,2,3,4-tetrahydro-naphthalen-1-ylamino]-propyl}-acetamide, N-{1-(3,5-Difluoro-benzyl)-2-hydroxy-3-[7-(6-methoxy-pyridazin-3-yl)-1,2,3,4-tetrahydro-naphthalen-1-ylamino]-propyl}-acetamide, N-{1-(3,5-Difluoro-benzyl)-2-hydroxy-3-[7-(4-methyl-pyridin-3-yl)-1,2,3,4-tetrahydro-naphthalen-1-ylamino]-propyl}-acetamide, N-[1-(3,5-Difluoro-benzyl)-2-hydroxy-3-(7-pyrazin-2-yl-1,2,3,4-tetrahydro-naphthalen-1-ylamino)-propyl]-acetamide, N-{1-(3,5-Difluoro-benzyl)-2-hydroxy-3-[7-(5-methyl-thiophen-2-yl)-1,2,3,4-tetrahydro-naphthalen-1-ylamino]-propyl}-acetamide, N-[1-(3,5-Difluoro-benzyl)-2-hydroxy-3-(7-thiazol-2-yl-1,2,3,4-tetrahydro-naphthalen-1-ylamino)-propyl]-acetamide, N-[1-(3,5-Difluoro-benzyl)-2-hydroxy-3-(7-thiophen-3-yl-1,2,3,4-tetrahydro-naphthalen-1-ylamino)-propyl]-acetamide, N-{l -(3,5-Difluoro-benzyl)-2-hydroxy-3-[7-(1-methyl-1H-imidazol-2-yl)-1,2,3,4-tetrahydro-naphthalen-1-ylamino]-propyl}-acetamide, N-[1-(3,5-Difluoro-benzyl)-2-hydroxy-3-(7-thiophen-2-yl-1,2,3,4-tetrahydro-naphthalen-1-ylamino)-propyl]-acetamide, N-(1-(3,5-Difluoro-benzyl)-2-hydroxy-3-[7-(3-methyl-thiophen-2-yl)-1,2,3,4-tetrahydro-naphthalen-1-ylamino]-propyl}-acetamide, N-[3-[5-(3-Amino-phenyl)-7-ethyl-1,2,3,4-tetrahydro-naphthalen-1-ylamino]-1-(3,5-difluoro-benzyl)-2-hydroxy-propyl]-acetamide, N-[1-(3,5-Difluoro-benzyl)-3-(7-ethyl-5-thiazol-2-yl-1,2,3,4-tetrahydro-naphthalen-1-ylamino)-2-hydroxy-propyl]-acetamide, N-[1-(3,5-Difluoro-benzyl)-3-(7-ethyl-5-pyridin-2-yl-1,2,3,4-tetrahydro-naphthalen-1-ylamino)-2-hydroxy-propyl]-acetamide, N-{1-(3,5-Difluoro-benzyl)-3-[7-ethyl-5-(3-methyl-pyridin-2-yl)-1,2,3,4-tetrahydro-naphthalen-1-ylamino]-2-hydroxy-propyl}-acetamide, N-{1-(3,5-Difluoro-benzyl)-3-[7-ethyl-5-(4-methyl-pyridin-2-yl)-1,2,3,4-tetrahydro-naphthalen-1-ylamino]-2-hydroxy-propyl}-acetamide, N-{1-(4-Benzyloxy-3-fluoro-benzyl)-3-[7-(2,2-dimethyl-propyl)-1,2,3,4-tetrahydro-naphthalen-1-ylamino]-2-hydroxy-propyl}-acetamide, N-[3-[7-(2,2-Dimethyl-propyl)-1,2,3,4-tetrahydro-naphthalen-1-ylamino]-1-(3-fluoro-4-hydroxy-benzyl)-2-hydroxy-propyl]-acetamide, N-{1-(3,5-Difluoro-benzyl)-3-[6-(2,2-dimethyl-propyl)-1-methyl-1,2,3,4-tetrahydro-quinolin-4-ylamino]-2-hydroxy-propyl}-acetamide, N-{1-(3,5-Difluoro-benzyl)-3-[7-(2,2-dimethyl-propyl)-4-oxo-1,2,3,4-tetrahydro-naphthalen-1-ylamino]-2-hydroxy-propyl}-acetamide, N-{1-(3,5-Difluoro-benzyl)-3-[7-(2,2-dimethyl-propyl)-5-ethyl-1,2,3,4-tetrahydro-naphthalen-1-ylamino]-2-hydroxy-propyl}-acetamide, N-{1-(3,5-Difluoro-benzyl)-3-[7-(2,2-dimethyl-propyl)-1,2,3,4-tetrahydro-naphthalen-1-ylamino]-2-hydroxy-propyl}-acetamide, N-(1-(3,5-Difluoro-benzyl)-3-[6-(2,2-dimethyl-propyl)-1,2,3,4-tetrahydro-quinolin-4-ylamino]-2-hydroxy-propyl}-acetamide, N-{1-(3,5-Difluoro-benzyl)-3-[7-(2,2-dimethyl-propyl)-1-methyl-1,2,3,4-tetrahydro-naphthalen-1-ylamino]-2-hydroxy-propyl}-acetamide, N-{1-(3,5-Difluoro-benzyl)-3-[7-(2,2-dimethyl-propyl)-1,2,3,4-tetrahydro-naphthalen-1-ylamino]-2-hydroxy-propyl}-2-fluoro-acetamide, N-[1-(3,5-Difluoro-benzyl)-2-hydroxy-3-(7-propyl-1,2,3,4-tetrahydro-naphthalen-1-ylamino)-propyl]-acetamide, N-[1-(3,5-Difluoro-benzyl)-2-hydroxy-3-(6-isopropyl-2-oxo-1,2,3,4-tetrahydro-quinolin-4-ylamino)-propyl]-acetamide, N-[1-(3,5-Difluoro-benzyl)-2-hydroxy-3-(7-isopropyl-3-oxo-1,2,3,4-tetrahydro-naphthalen-1-ylamino)-propyl]-acetamide, N-[1-(3,5-Difluoro-benzyl)-2-hydroxy-3-(3-hydroxy-7-isopropyl-3-methyl-1,2,3,4-tetrahydro-naphthalen-1-ylamino)-propyl]-acetamide, N-[3-(3-Acetylamino-7-isopropyl-1,2,3,4-tetrahydro-naphthalen-1-ylamino)-1-(3,5-difluoro-benzyl)-2-hydroxy-propyl]-acetamide, N-[1-(3,5-Difluoro-benzyl)-2-hydroxy-3-(7-isopropyl-3-methanesulfonylamino-1,2,3,4-tetrahydro-naphthalen-1-ylamino)-propyl]-acetamide, N-[3-[7-(2,2-Dimethyl-propyl)-1,2,3,4-tetrahydro-naphthalen-1-ylamino]-2-hydroxy-1-(5-hydroxy-pyridin-2-ylmethyl)-propyl]-acetamide, N-[1-(3,5-Difluoro-benzyl)-2-hydroxy-3-(1,2,3,4-tetrahydro-naphthalen-1-ylamino)-propyl]-acetamide, N-[1-(3,5-Difluoro-benzyl)-2-hydroxy-3-(7-methoxy-1,2,3,4-tetrahydro-naphthalen-1-ylamino)-propyl]-acetamide, N-[1-(3,5-Difluoro-benzyl)-3-(7-ethyl-1,2,3,4-tetrahydro-naphthalen-1-ylamino)-2-hydroxy-propyl]-acetamide, N-[1-(3,5-Difluoro-benzyl)-3-(6-ethyl-1-methyl-1,2,3,4-tetrahydro-quinolin-4-ylamino)-2-hydroxy-propyl]-acetamide, N-[1-(3,5-Difluoro-benzyl)-3-(7-dimethylaminomethyl-1,2,3,4-tetrahydro-naphthalen-1-ylamino)-2-hydroxy-propyl]-acetamide, N-[3-(7-Bromo-1,2,3,4-tetrahydro-naphthalen-1-ylamino)-1-(3,5-difluoro-benzyl)-2-hydroxy-propyl]-acetamide, N-{1-(3,5-Difluoro-benzyl)-3-[6-carbobenzoxy-1,2,3,4-tetrahydro-quinolin-4-ylamino]-2-hydroxy-propyl}-acetamide, N-[1-(3,5-Difluoro-benzyl)-3-(7-ethyl-2,2-dimethyl-1,2,3,4-tetrahydro-naphthalen-1-ylamino)-2-hydroxy-propyl]-acetamide, N-[1-(3,5-Difluoro-benzyl)-2-hydroxy-3-(7-isobutyl-1,2,3,4-tetrahydro-naphthalen-1-ylamino)-propyl]-acetamide, N-[3-(5-Bromo-7-ethyl-1,2,3,4-tetrahydro-naphthalen-1-ylamino)-1-(3,5-difluoro-benzyl)-2-hydroxy-propyl]-acetamide, N-[3-(5,7-Diethyl-1,2,3,4-tetrahydro-naphthalen-1-ylamino)-1-(3,5-difluoro-benzyl)-2-hydroxy-propyl]-acetamide, N-[3-(5-Butyl-7-ethyl-1,2,3,4-tetrahydro-naphthalen-1-ylamino)-1-(3,5-difluoro-benzyl)-2-hydroxy-propyl]-acetamide, N-[1-(3-Butoxy-5-fluoro-benzyl)-3-(7-ethyl-1,2,3,4-tetrahydro-naphthalen-1-ylamino)-2-hydroxy-propyl]-acetamide, N-[1-(3-Benzyloxy-5-fluoro-benzyl)-3-(7-ethyl-1,2,3,4-tetrahydro-naphthalen-1-ylamino)-2-hydroxy-propyl]-acetamide, N-[3-(7-Ethyl-1,2,3,4-tetrahydro-naphthalen-1-ylamino)-1-(3-fluoro-5-hydroxy-benzyl)-2-hydroxy-propyl]-acetamide, N-[1-(3,5-Difluoro-benzyl)-3-(7-ethyl-5-propyl-1,2,3,4-tetrahydro-naphthalen-1-ylamino)-2-hydroxy-propyl]-acetamide, N-[1-(3,5-Difluoro-benzyl)-3-(7-ethyl-5-isobutyl-1,2,3,4-tetrahydro-naphthalen-1-ylamino)-2-hydroxy-propyl]-acetamide, N-{1-(3,5-Difluoro-benzyl)-3-[7-(2,2-dimethyl-propyl)-2-hydroxymethyl-1,2,3,4-tetrahydro-naphthalen-1-ylamino]-2-hydroxy-propyl}-acetamide, N-{1l-(3,5-Difluoro-benzyl)-3-[7-ethyl-5-(5-methyl-pyridin-2-yl)-1,2,3,4-tetrahydro-naphthalen-1-ylamino]-2-hydroxy-propyl}-acetamide, N-{1-(3,5-Difluoro-benzyl)-3-[7-ethyl-5-(6-methyl-pyridin-2-yl)-1,2,3,4-tetrahydro-naphthalen-1-ylamino]-2-hydroxy-propyl}-acetamide, N-[3-(7-Butyl-1,2,3,4-tetrahydro-naphthalen-1-ylamino)-1-(3,5-difluoro-benzyl)-2-hydroxy-propyl]-acetamide, N-[3-(5-Cyano-7-ethyl-1,2,3,4-tetrahydro-naphthalen-1-ylamino)-1-(3,5-difluoro-benzyl)-2-hydroxy-propyl]-acetamide, N-[1-(3,5-Difluoro-benzyl)-3-(6-ethyl-1,2,3,4-tetrahydro-quinolin-4-ylamino)-2-hydroxy-propyl]-acetamide, N-[1-(3,5-Difluoro-benzyl)-3-(7-ethyl-1-methyl-1,2,3,4-tetrahydro-naphthalen-1-ylamino)-2-hydroxy-propyl]-acetamide, N-[3-(7-sec-Butyl-1,2,3,4-tetrahydro-naphthalen-1-ylamino)-1-(3,5-difluoro-benzyl)-2-hydroxy-propyl]-acetamide, N-(1-(3,5-difluorobenzyl)-2-hydroxy-3-{[6-neopentyl-1,2,3,4-tetrahydroquinolin-4-yl]amino}propyl)acetamide, N-(1-(3,5-difluorobenzyl)-2-hydroxy-3-{[6-isobutyl-1,2,3,4-tetrahydroquinolin-4-yl]amino}propyl)acetamide, N-(1-(3,5-difluorobenzyl)-2-hydroxy-3-{[6-isopropyl-1,2,3,4-tetrahydroquinolin-4-yl]amino}propyl)acetamide, N-[3-{[6-tert-butyl-1,2,3,4-tetrahydroquinolin-4-yl]amino}-1-(3,5-difluorobenzyl)-2-hydroxypropyl]acetamide, N-(1-(3,5-difluorobenzyl)-3-{[6-ethyl-1,2,3,4-tetrahydroquinolin-4-yl]amino}-2-hydroxypropyl)acetamide, N-(1-(3,5-difluorobenzyl)-3-{[7-fluoro-6-isopropyl-1,2,3,4-tetrahydroquinolin-4-yl]amino}-2-hydroxypropyl)acetamide, N-[3-{[6-tert-butyl-7-fluoro-1,2,3,4-tetrahydroquinolin-4-yl]amino}-1-(3,5-difluorobenzyl)-2-hydroxypropyl]acetamide, N-(1-(3,5-difluorobenzyl)-3-{[7-fluoro-6-isobutyl-1,2,3,4-tetrahydroquinolin-4-yl]amino}-2-hydroxypropyl)acetamide, N-(1-(3,5-difluorobenzyl)-3-{[7-fluoro-6-neopentyl-1,2,3,4-tetrahydroquinolin-4-yl]amino}-2-hydroxypropyl)acetamide, N-(1-(3,5-difluorobenzyl)-2-hydroxy-3-{[1-methyl-6-neopentyl-1,2,3,4-tetrahydroquinolin-4-yl]amino}propyl)acetamide, N-(1-(3,5-difluorobenzyl)-2-hydroxy-3-{[6-isobutyl-1-methyl-1,2,3,4-tetrahydroquinolin-4-yl]amino}propyl)acetamide, N-(1-(3,5-difluorobenzyl)-2-hydroxy-3-{[6-isopropyl-1-methyl-1,2,3,4-tetrahydroquinolin-4-yl]amino}propyl)acetamide, N-[3-{[6-tert-butyl-1-methyl-1,2,3,4-tetrahydroquinolin-4-yl]amino}-1-(3,5-difluorobenzyl)-2-hydroxypropyl]acetamide, N-[3-{[6-tert-butyl-1-(2-hydroxyethyl)-1,2,3,4-tetrahydroquinolin-4-yl]amino}-1-(3,5-difluorobenzyl)-2-hydroxypropyl]acetamide, N-(1-(3,5-difluorobenzyl)-2-hydroxy-3-{[1-(2-hydroxyethyl)-6-isopropyl-1,2,3,4-tetrahydroquinolin-4-yl]amino}propyl)acetamide, N-(1-(3,5-difluorobenzyl)-2-hydroxy-3-{[1-(2-hydroxyethyl)-6-isobutyl-1,2,3,4-tetrahydroquinolin-4-yl]amino}propyl)acetamide, N-(1-(3,5-difluorobenzyl)-2-hydroxy-3-{[1-(2-hydroxyethyl)-6-neopentyl-1,2,3,4-tetrahydroquinolin-4-yl]amino}propyl)acetamide, N-[3-{[1-acetyl-6-neopentyl-1,2,3,4-tetrahydroquinolin-4-yl]amino}-1-(3,5-difluorobenzyl)-2-hydroxypropyl]acetamide, N-[3-{[1-acetyl-6-isobutyl-1,2,3,4-tetrahydroquinolin-4-yl]amino)-1-(3,5-difluorobenzyl)-2-hydroxypropyl]acetamide, N-[3-{[1-acetyl-6-isopropyl-1,2,3,4-tetrahydroquinolin-4-yl]amino}-1-(3,5-difluorobenzyl)-2-hydroxypropyl]acetamide, N-[3-{[1-acetyl-6-tert-butyl-1,2,3,4-tetrahydroquinolin-4-yl]amino}-1-(3,5-difluorobenzyl)-2-hydroxypropyl]acetamide, N-[3-{[6-tert-butyl-1-(cyanomethyl)-1,2,3,4-tetrahydroquinolin-4-yl]amino}-1-(3,5-difluorobenzyl)-2-hydroxypropyl]acetamide, N-[3-{[1-(cyanomethyl)-6-isopropyl-1,2,3,4-tetrahydroquinolin-4-yl]amino}-1-(3,5-difluorobenzyl)-2-hydroxypropyl]acetamide, N-[3-{[1-(cyanomethyl)-6-isobutyl-1,2,3,4-tetrahydroquinolin-4-yl]amino)-1-(3,5-difluorobenzyl)-2-hydroxypropyl]acetamide, N-[3-{[1-(cyanomethyl)-6-neopentyl-1,2,3,4-tetrahydroquinolin-4-yl]amino}-1-(3,5-difluorobenzyl)-2-hydroxypropyl]acetamide, N-(1-(3,5-difluorobenzyl)-2-hydroxy-3-{[6-(1-hydroxy-2,2-dimethylpropyl)-1,2,3,4-tetrahydroquinolin-4-yl]amino}propyl)acetamide, N-(1-(3,5-difluorobenzyl)-2-hydroxy-3-{[6-(1-hydroxy-2,2-dimethylpropyl)-1-methyl-1,2,3,4-tetrahydroquinolin-4-yl]amino)propyl)acetamide, N-(1-(3,5-difluorobenzyl)-3-{[2,2-dimethyl-6-neopentyl-1,2,3,4-tetrahydroquinolin-4-yl]amino}-2-hydroxypropyl)acetamide, N-(1-(3,5-difluorobenzyl)-2-hydroxy-3-{[1,2,2-trimethyl-6-neopentyl-1,2,3,4-tetrahydroquinolin-4-yl]amino}propyl)acetamide, N-(1-(3,5-difluorobenzyl)-3-{[1,4-dimethyl-6-neopentyl-1,2,3,4-tetrahydroquinolin-4-yl]amino}-2-hydroxypropyl)acetamide, N-(1-(3,5-difluorobenzyl)-2-hydroxy-3-{[4-methyl-6-neopentyl-1,2,3,4-tetrahydroquinolin-4-yl]amino}propyl)acetamide, N-(1-(3,5-difluorobenzyl)-2-hydroxy-3-{[6-isobutyl-4-methyl-1,2,3,4-tetrahydroquinolin-4-yl]amino}propyl)acetamide, N-(1-(3,5-difluorobenzyl)-2-hydroxy-3-{[6-isobutyl-1,4-dimethyl-1,2,3,4-tetrahydroquinolin-4-yl]amino}propyl)acetamide, N-[3-[(6-tert-butoxy-1,2,3,4-tetrahydroquinolin-4-yl)amino]-1-(3,5-difluorobenzyl)-2-hydroxypropyl]acetamide, N-[3-[(6-tert-butoxy-4-methyl-1,2,3,4-tetrahydroquinolin-4-yl)amino]-1-(3,5-difluorobenzyl)-2-hydroxypropyl]acetamide, N-[3-[(6-tert-butoxy-4,8-dimethyl-1,2,3,4-tetrahydroquinolin-4-yl)amino]-1-(3,5-difluorobenzyl)-2-hydroxypropyl]acetamide, N-{1-(3,5-difluorobenzyl)-2-hydroxy-3-[(4-methyl-6-neopentyl-1,2,3,4-tetrahydroquinolin-4-yl)amino]propyl}acetamide, N-{1-(3,5-difluorobenzyl)-3-[(4,8-dimethyl-6-neopentyl-1,2,3,4-tetrahydroquinolin-4-yl)amino]-2-hydroxypropyl}acetamide, N-{1-(3,5-difluorobenzyl)-2-hydroxy-3-[(8-methyl-6-neopentyl-1,2,3,4-tetrahydroquinolin-4-yl)amino]propyl}acetamide, N-(1-(3,5-difluorobenzyl)-2-hydroxy-3-{[6-(2-hydroxy-2-methylpropyl)-8-methyl-1,2,3,4-tetrahydroquinolin-4-yl]amino}propyl)acetamide, N-(1-(3,5-difluorobenzyl)-2-hydroxy-3-{[6-(2-hydroxy-2-methylpropyl)-4-methyl-1,2,3,4-tetrahydroquinolin-4-yl]amino}propyl)acetamide, N-(1-(3,5-difluorobenzyl)-2-hydroxy-3-{[6-(2-hydroxy-2-methylpropyl)-1,2,3,4-tetrahydroquinolin-4-yl]amino}propyl)acetamide, N-(1-(3,5-difluorobenzyl)-2-hydroxy-3-{[6-(1-hydroxy-2,2-dimethylpropyl)-4-methyl-1,2,3,4-tetrahydroquinolin-4-yl]amino)propyl)acetamide, N-{1-(3,5-difluorobenzyl)-2-hydroxy-3-[(5-isobutyl-2-pyridin-3-ylbenzyl)amino]propyl}acetamide, N-{1-(3,5-difluorobenzyl)-2-hydroxy-3-[(5-isobutyl-2-pyridin-4-ylbenzyl)amino]propyl}acetamide, N-(1-(3,5-difluorobenzyl)-2-hydroxy-3-{[5-isobutyl-2-(6-methoxypyridin-3-yl)benzyl]amino}propyl) acetamide, N-(1-(3,5-difluorobenzyl)-2-hydroxy-3-{[5-isobutyl-2-(5-methoxypyridin-3-yl)benzyl]amino}propyl) acetamide, N-[3-(5,7-Diethyl-1,2,3,4-tetrahydronaphthalen-1-ylamino)-1-(3,5-difluorobenzyl)-2-hydroxypropyl]-acetamide, N-[1-(3,5-Difluorobenzyl)-3-(7-ethyl-5-propyl-1,2,3,4-tetrahydro-naphthalen-1-ylamino)-2-hydroxypropyl]-acetamide, N-[1-(3,5-Difluorobenzyl)-3-(7-ethyl-5-isobutyl-1,2,3,4-tetrahydronaphthalen-1-ylamino)-2-hydroxypropyl]-acetamide, N-{1-(3,5-Difluoro-benzyl)-2-hydroxy-3-(7-pyrimidin-5-yl-1,2,3,4-tetrahydro-naphthalen-1-ylamino)-propyl}-acetamide, N-{1-(3,5-Difluoro-benzyl)-2-hydroxy-3-(7-pyridin-2-yl-1,2,3,4-tetrahydro-naphthalen-1-ylamino)-propyl}-acetamide, N-{1-(3,5-Difluoro-benzyl)-2-hydroxy-3-(7-pyridin-3-yl-1,2,3,4-tetrahydro-naphthalen-1-ylamino)-propyl}-acetamide, N-{1-(3,5-Difluoro-benzyl)-2-hydroxy-3-(7-pyridin-4-yl-1,2,3,4-tetrahydro-naphthalen-1-ylamino)-propyl}-acetamide, N-{1-(3,5-Difluoro-benzyl)-2-hydroxy-3-(7-pyrazin-2-yl-1,2,3,4-tetrahydro-naphthalen-1-ylamino)-propyl}-acetamide, N-{1-(3,5-Difluoro-benzyl)-2-hydroxy-3-[7-(3,6-dimethyl-pyrazin-2-yl)-1,2,3,4-tetrahydro-naphthalen-1-ylamino]-2-hydroxy-propyl}-acetamide, N-{1-(3,5-Difluoro-benzyl)-2-hydroxy-3-(7-furan-2-yl-1,2,3,4-tetrahydro-naphthalen-1-ylamino)-2-hydroxy-propyl}-acetamide, N-{1-(3,5-Difluoro-benzyl)-2-hydroxy-3-(7-thiazol-2-yl-1,2,3,4-tetrahydro-naphthalen-1-ylamino)-propyl}-acetamide, N-{1-(3,5-Difluoro-benzyl)-2-hydroxy-3-(7-thiophen-3-yl-1,2,3,4-tetrahydro-naphthalen-1-ylamino)-propyl}-acetamide, N-{1-(3,5-Difluoro-benzyl)-2-hydroxy-3-(7-styryl-1,2,3,4-tetrahydro-naphthalen-1-ylamino)-propyl}-acetamide, N-{1-(3,5-Difluoro-benzyl)-2-hydroxy-3-[7-(3,5-dimethyl-isoxazol-4-yl)-1,2,3,4-tetrahydro-naphthalen-1-ylamino]-2-hydroxy-propyl}-acetamide, N-(1-(3,5-Difluoro-benzyl)-2-hydroxy-3-(7-thiophen-2-yl-1,2,3,4-tetrahydro-naphthalen-1-ylamino)-propyl}-acetamide, N-{1-(3,5-Difluoro-benzyl)-2-hydroxy-3-[7-(5-ethyl-pyrimidin-2-yl)-1,2,3,4-tetrahydro-naphthalen-1-ylamino]-2-hydroxy-propyl}-acetamide, N-{1-(3,5-Difluoro-benzyl)-2-hydroxy-3-(7-isopropenyl-1,2,3,4-tetrahydro-naphthalen-1-ylamino)-propyl}-acetamide, N-[3-{1-[3-(5-Acetyl-thiophen-2-yl)-phenyl]-cyclopropylamino)-1-(3,5-difluoro-benzyl)-2-hydroxy-propyl]-acetamide, N-{1-(3,5-Difluoro-benzyl)-2-hydroxy-3-[1-(3-thiophen-3-yl-phenyl)-cyclopropylamino]-propyl}-acetamide, N-(1-(3,5-Difluoro-benzyl)-2-hydroxy-3-{1-[3-(6-methoxy-pyridin-3-yl)-phenyl]-cyclopropylamino}-propyl)-acetamide, N-{1-(3,5-Difluoro-benzyl)-3-[1-(3-furan-3-yl-phenyl)-cyclopropylamino]-2-hydroxy-propyl}-acetamide, N-(1-(3,5-Difluoro-benzyl)-3-{1-[3-(3,5-dimethyl-isoxazol-4-yl)-phenyl]-cyclopropylamino}-2-hydroxy-propyl)-acetamide, N-[3-(6-tert-Butyl-1,2,3,4-tetrahydro-quinolin-4-ylamino)-1-(3,5-difluoro-benzyl)-2-hydroxy-propyl]-acetamide, N-[1-(3,5-Difluoro-2-methoxy-benzyl)-3-(7-ethyl-1,2,3,4-tetrahydro-naphthalen-1-ylamino)-2-hydroxy-propyl]-acetamide, N-[1-(3,5-Difluoro-benzyl)-2-hydroxy-3-(7-propyl-1,2,3,4-tetrahydro-naphthalen-1-ylamino)-propyl]-2-fluoro-acetamide, N-[1-(3,5-Difluoro-benzyl)-2-hydroxy-3-(1-methyl-7-propyl-1,2,3,4-tetrahydro-naphthalen-1-ylamino)-propyl]-acetamide, N-[3-(7-tert-Butyl-1,2,3,4-tetrahydro-naphthalen-1-ylamino)-1-(3-fluoro-5-hydroxy-benzyl)-2-hydroxy-propyl]-acetamide, N-[1-(3-Benzyloxy-5-fluoro-benzyl)-3-(7-tert-butyl-1,2,3,4-tetrahydro-naphthalen-1-ylamino)-2-hydroxy-propyl]-acetamide, N-[3-{[5-(3-aminophenyl)-7-ethyl-1,2,3,4-tetrahydronaphthalen-1-yl]amino}-1-(3,5-difluorobenzyl)-2-hydroxypropyl]acetamide, N-(1-(3,5-difluorophenyl)-3-hydroxy-4-(7-propyl-1,2,3,4-tetrahydronaphthalen-1-ylamino)butan-2-yl)acetamide, N-(4-(7-tert-butyl-1,2,3,4-tetrahydronaphthalen-1-ylamino)-1-(3-fluoro-4-hydroxyphenyl)-3-hydroxybutan-2-yl)acetamide, N-(1-(3-fluoro-4-hydroxyphenyl)-3-hydroxy-4-(7-neopentyl-1,2,3,4-tetrahydronaphthalen-1-ylamino)butan-2-yl)acetamide, and N-(4-(7-ethyl-1-methyl-1,2,3,4-tetrahydronaphthalen-1-ylamino)-1-(3-fluoro-4-hydroxyphenyl)-3-hydroxybutan-2-yl)acetamide, or a pharmaceutically acceptable salt thereof.

In another embodiment, the at least one compound of formula (I) is N-[1-(3,5-Difluorobenzyl)-3-(7-ethyl-1,2,3,4-tetrahydro-naphthalen-1-ylamino)-2-hydroxy-propyl]-N′,N′-dimethyl-succinamide, Pent-3-enoic acid [1-(3,5-difluoro-benzyl)-3-(7-ethyl-1,2,3,4-tetrahydro-naphthalen-1-ylamino)-2-hydroxy-propyl]-amide, Hex-3-enoic acid [1-(3,5-difluoro-benzyl)-3-(7-ethyl-1,2,3,4-tetrahydro-naphthalen-1-ylamino)-2-hydroxy-propyl]-amide, 3-Allyloxy-N-[1-(3,5-difluoro-benzyl)-3-(7-ethyl-1,2,3,4-tetrahydro-naphthalen-1-ylamino)-2-hydroxy-propyl]-propionamide, N-(1-(3,5-difluorobenzyl)-3-{[7-ethyl-1,2,3,4-tetrahydronaphthalen-1-yl]amino}-2-hydroxypropyl)ethanethioamide hydrochloride, N-[1-(3,5-Difluorobenzyl)-3-(7-ethyl-1,2,3,4-tetrahydro-naphthalen-1-ylamino)-2-hydroxy-propyl]-methanesulfonamide, tert-butyl 1-(3,5-difluorobenzyl)-3-[(6-ethyl-1-methyl-1,2,3,4-tetrahydroquinolin-4-yl)amino]-2-hydroxypropylcarbamate, {1-(3,5-Difluoro-benzyl)-3-[6-(2,2-dimethyl-butyl)-1-methyl-1,2,3,4-tetrahydro-quinolin-4-ylamino]-2-hydroxy-propyl}-carbamic acid tert-butyl ester, {1-(3,5-Difluoro-benzyl)-3-[6-(2,2-dimethyl-propyl)-1-methyl-1,2,3,4-tetrahydro-quinolin-4-ylamino]-2-hydroxy-propyl}-carbamic acid tert-butyl ester, N-[1-(3,5-Difluoro-benzyl)-3-(7-ethyl-1,2,3,4-tetrahydro-naphthalen-1-ylamino)-2-hydroxy-propyl]-2,2-difluoro-acetamide, N-[1-(3,5-Difluoro-benzyl)-3-(7-ethyl-1,2,3,4-tetrahydro-naphthalen-1-ylamino)-2-hydroxy-propyl]-2-hydroxy-acetamide, N-[1-(3,5-Difluorobenzyl)-3-(7-ethyl-1,2,3,4-tetrahydro-naphthalen-1-ylamino)-2-hydroxy-propyl]-propionamide, 5-Oxo-hexanoic acid [1-(3,5-difluorobenzyl)-3-(7-ethyl-1,2,3,4-tetrahydro-naphthalen-1-ylamino)-2-hydroxy-propyl]-amide, N-(1-(3,5-difluorophenyl)-4-(7-ethyl-1,2,3,4-tetrahydronaphthalen-1-ylamino)-3-hydroxybutan-2-yl)methanesulfonamide, N-(1-(3,5-difluorophenyl)-3-hydroxy-4-(7-neopentyl-1,2,3,4-tetrahydronaphthalen-1-ylamino)butan-2-yl)methanesulfonamide,N-(1-(3,5-difluorophenyl)-3-hydroxy-4-(7-neopentyl-1,2,3,4-tetrahydronaphthalen-1-ylamino)butan-2-yl)-3-(methylsulfonamido)benzamide, N-(1-(3,5-difluorophenyl)-3-hydroxy-4-(7-neopentyl-1,2,3,4-tetrahydronaphthalen-1-ylamino)butan-2-yl)-3-(N-methylmethylsulfonamido)benzamide, 2-(3,5-difluorobenzyl)-4-(7-ethyl-1,2,3,4-tetrahydronaphthalen-1-ylamino)-3-hydroxy-N-methylbutanamide, 2-(3,5-difluoro-2-((methylamino)methyl)benzyl)-4-(7-ethyl-1,2,3,4-tetrahydronaphthalen-1-ylamino)-3-hydroxy-N-methylbutanamide, 4-(7-ethyl-1,2,3,4-tetrahydronaphthalen-1-ylamino)-3-hydroxy-N-methyl-2-((4-propylthiophen-2-yl)methyl)butanamide, and Pentanoic acid [1-(3,5-difluoro-benzyl)-3-(7-ethyl-1,2,3,4-tetrahydro-naphthalen-1-ylamino)-2-hydroxy-propyl]-amide, or a pharmaceutically acceptable salt thereof.

An embodiment of the present invention is compounds of formula (I), or pharmaceutically acceptable salts thereof, wherein R and R′ are independently selected from hydrogen and —C₁-C₁₀ alkyl (substituted with at least one group selected from OH).

In another embodiment, R_{B} is selected from —CF₃, —(C(O))₀₋₁—(O)₀₋₁-alkyl, and —C(O)—OH.

In another embodiment, R_{N} is selected alkyl-R₁₀₀, —NH₂, —OH, —(CRR′)₁₋₆′P(O)(O-alkyl)₂, and alkyl-O-alkyl-C(O)OH.

In another embodiment, R₄and R₄ are independently selected from —OH.

In another embodiment, R₁₀₀ and R′₁₀₀ are independently selected from alkoxy.

In another embodiment, R₁₀₁ and R′₁₀₁ are independently selected from —(C(O))₀₋₁—(O)₀₋₁-alkyl and —C(O)—OH.

In another embodiment, R₁₁₅ is —NH—C(O)-(alkyl).

In another embodiment, R₂₀₀ is —(CH₂)₀₋₄—C(O)—NH(R₂₁₅).

In another embodiment, R₂₀₅ is selected from —(CH₂)₀₋₆—C(O)—R₂₃₅, —(CH₂)₀₋₄—N(H or R₂₁₅)—SO₂—R₂₃₅, —CN, and —OCF₃.

In another embodiment, R₂₁₀ is selected from heterocycloalkyl, heteroaryl, —(CO)₀₋₁R₂₁₅, —(CO)₀₋₁R₂₂₀, —(CH₂)₀₋₄—NR₂₃₅R₂₄₀, —(CH₂)₀₋₄—NR₂₃₅(alkoxy), —(CH₂)₀₋₄—S—(R₂₁₅), —(CH₂)₀₋₆—OH, —(CH₂)₀₋₆—CN, —(CH₂)₀₋₄—NR₂₃₅—C(O)H, —(CH₂)₀₋₄—NR₂₃₅—C(O)-(alkoxy), —(CH₂)₀₋₄—NR₂₃₅—C(O)—R₂₄₀, and—C(O)—NHR_{215.}

In another embodiment, R₂₃₅ and R₂₄₀ are independently selected from —OH, —CF₃, —OCH₃, —NH—CH₃, —N(CH₃)₂, —(CH₂)₀₋₄—C(O)—(H or alkyl).

In another embodiment, D is cycloalkyl.

In another embodiment, E₁ is C₁-C₄ alkyl.

In another embodiment, V is cycloalkyl.

In another embodiment, at least one carbon of the aryl, heteroaryl, cycloalkyl, and heterocycloalkyl groups included within V and V′ are optionally replaced with a group selected form —C(O)—, —C(S)—, —C(═N—H)—, —C(═N—OH)—, —C(═N-alkyl)-, and —C(═N—O-alkyl) —(C(O))₀₋₁—(O)₀₋₁-alkyl, and C(O)—OH.

In another embodiment, the formula (I) compounds are selected from {1-(3,5-Difluoro-benzyl)-3-[6-(2,2-dimethyl-propyl)-1-methyl-1,2,3,4-tetrahydro-quinolin-4-ylamino]-2-hydroxy-propyl}-carbamic acid tert-butyl ester, N-(1-(3,5-difluorophenyl)-3-hydroxy-4-(7-neopentyl-1,2,3,4-tetrahydronaphthalen-1-ylamino)butan-2-yl)-3-(methylsulfonamido)benzamide, N-(1-(3,5-difluorophenyl)-3-hydroxy-4-(7-neopentyl-1,2,3,4-tetrahydronaphthalen-1-ylamino)butan-2-yl)-3-(N-methylmethylsulfonamido)benzamide.

The present invention encompasses methods of treatment using compounds with structural characteristics designed for interacting with their target molecules. Such characteristics include at least one moiety capable of interacting with at least one subsite of beta-secretase. Such characteristics also include at least one moiety capable of enhancing the interaction between the target and at least one subsite of beta-secretase.

Accordingly, the compounds of formula (I) incorporate bicyclic moieties, for example tetrahydroquinoline or tetralin, at R_{C}. Compounds with such moieties possess structural characteristics that corresponds to desired properties such as increased bioavailability, efficacy, and/or selectivity.

It is preferred that the compounds of formula (I) are efficacious. For example, it is preferred that the compounds of formula (I) decrease the level of beta-secretase using low dosages of the compounds. Preferably, the compounds of formula (I) decrease the level of A-beta by at least 10% using dosages of about 100 mg/kg. It is more preferred that the compounds of formula (I) decrease the level of A-beta by at least 10% using dosages of less than 100 mg/kg. It is also more preferred that the compounds of formula (I) decrease the level of A-beta by greater than 10% using dosages of about 100 mg/kg. It is most preferred that the compounds of formula (I) decrease the level of A-beta by greater than 10% using dosages of less than 100 mg/kg.

Another embodiment of the present invention is to provide methods of preventing or treating conditions associated with amyloidosis using compounds with increased oral bioavailability (increased F values).

Accordingly, an embodiment of the present invention is also directed to methods for preventing or treating conditions associated with amyloidosis, comprising administering to a host a therapeutically effective amount of at least one compound of formula (I), or a pharmaceutically acceptable salt thereof, wherein R₁, R₂, and R_{C} are as previously defined, and wherein the compound has an F value of at least 10%.

Investigation of potential beta-secretase inhibitors produced compounds with increased selectivity for beta-secretase over other aspartyl proteases such as cathepsin D (catD), cathepsin E (catE), HIV protease, and renin. Selectivity was calculated as a ratio of inhibition (IC₅₀) values in which the inhibition of beta-secretase was compared to the inhibition of other aspartyl proteases. A compound is selective when the IC₅₀ value (i.e., concentration required for 50% inhibition) of a desired target (e.g., beta-secretase). is less than the IC₅₀ value of a secondary target (e.g., catD). Alternatively, a compound is selective when its binding affinity is greater for its desired target (e.g., beta-secretase) versus a secondary target (e.g., catD). Accordingly, methods of treatment include administering selective compounds of formula (I) having a lower IC₅₀ value for inhibiting beta-secretase, or greater binding affinity for beta-secretase, than for other aspartyl proteases such as catD, catE, HIV protease, or renin. A selective compound is also capable of producing a higher ratio of desired effects to adverse effects, resulting in a safer method of treatment.

In another embodiment, the host is a cell.

In another embodiment, the host is an animal.

In another embodiment, the host is human.

In another embodiment, at least one compound of formula (I) is administered in combination with a pharmaceutically acceptable carrier or diluent.

In another embodiment, the pharmaceutical compositions comprising compounds of formula (I) can be used to treat a wide variety of disorders or conditions including Alzheimer's disease, Down's syndrome or Trisomy 21 (including mild cognitive impairment (MCI) Down's syndrome), hereditary cerebral hemorrhage with amyloidosis of the Dutch type, chronic inflammation due to amyloidosis, prion diseases (including Creutzfeldt-Jakob disease, Gerstmann-Straussler syndrome, kuru scrapie, and animal scrapie), Familial Amyloidotic Polyneuropathy, cerebral amyloid angiopathy, other degenerative dementias including dementias of mixed vascular and degenerative origin, dementia associated with Parkinson's disease, dementia associated with progressive supranuclear palsy and dementia associated with cortical basal degeneration, diffuse Lewy body type of Alzheimer's disease, and frontotemporal dementias with parkinsonism (FTDP).

In another embodiment, the condition is Alzheimer's disease.

In another embodiment, the condition is dementia.

When treating or preventing these diseases, the methods of the present invention can either employ the compounds of formula (I) individually or in combination, as is best for the patient.

In treating a patient displaying any of the conditions discussed above, a physician may employ a compound of formula (I) immediately and continue administration indefinitely, as needed. In treating patients who are not diagnosed as having Alzheimer's disease, but who are believed to be at substantial risk for it, the physician may start treatment when the patient first experiences early pre-Alzheimer's symptoms, such as memory or cognitive problems associated with aging. In addition, there are some patients who may be determined to be at risk for developing Alzheimer's disease through the detection of a genetic marker such as APOE4 or other biological indicators that are predictive for Alzheimer's disease and related conditions. In these situations, even though the patient does not have symptoms of the disease or condition, administration of the compounds of formula (I) may be started before symptoms appear, and treatment may be continued indefinitely to prevent or delay the onset of the disease. Similar protocols are provided for other diseases and conditions associated with amyloidosis, such as those characterized by dementia.

In an embodiment, the methods of preventing or treating conditions associated with amyloidosis, comprising administering to a host a composition comprising a therapeutically effective amount of at least one compound of formula (I), may include beta-secretase complexed with at least one compound of formula (I), or a pharmaceutically acceptable salt thereof.

An embodiment of the present invention is a method of preventing or treating the onset of Alzheimer's disease comprising administering to a patient a therapeutically effective amount of at least one compound of formula (I), or a pharmaceutically acceptable salt thereof, wherein R₁, R₂, and R_{C} are as previously defined.

Another embodiment of the present invention is a method of preventing or treating the onset of dementia comprising administering to a patient a therapeutically effective amount of at least one compound of formula (I), or a pharmaceutically acceptable salt thereof, wherein R₁, R₂, and R_{C} are as previously defined.

Another embodiment of the present invention is a method of preventing or treating conditions associated with amyloidosis by administering to a host an effective amount of at least one compound of formula (I), or a pharmaceutically acceptable salt thereof, wherein R₁, R₂, and R_{C} are as previously defined.

Another embodiment of the present invention is a method of preventing or treating Alzheimer's disease by administering to a host an effective amount of at least one compound of formula (I), or a pharmaceutically acceptable salt thereof, wherein R₁, R₂, and R_{C} are as previously defined.

Another embodiment of the present invention is a method of preventing or treating dementia by administering to a host an effective amount of at least one compound of formula (I), or a pharmaceutically acceptable salt thereof, wherein R₁, R₂, and R_{C} are as previously defined.

Another embodiment of the present invention is a method of inhibiting beta-secretase activity in a cell. This method comprises administering to the cell an effective amount of at least one compound of formula (I), or a pharmaceutically acceptable salt thereof, wherein R₁, R₂, and R_{C} are as previously defined.

Another embodiment of the present invention is a method of inhibiting beta-secretase activity in a host. This method comprises administering to the host an effective amount of at least one compound of formula (I), or a pharmaceutically acceptable salt thereof, wherein R₁, R₂, and R_{C} are as previously defined.

Another embodiment of the present invention is a method of inhibiting beta-secretase activity in a host. This method comprises administering to the host an effective amount of at least one compound of formula (I), or a pharmaceutically acceptable salt thereof, wherein R₁, R₂, and R_{C} are as previously defined, and wherein the host is a human.

Another embodiment of the present invention is methods of affecting beta-secretase-mediated cleavage of amyloid precursor protein in a patient, comprising administering a therapeutically effective amount of at least one compound of formula (I), or a pharmaceutically acceptable salt thereof, wherein R₁, R₂, and R_{C} are as previously defined.

Another embodiment of the present invention is a method of inhibiting cleavage of amyloid precursor protein at a site between Met596 and Asp597 (numbered for the APP-695 amino acid isotype), or at a corresponding site of an isotype or mutant thereof, comprising administering a therapeutically effective amount of at least one compound of formula (I), or a pharmaceutically acceptable salt thereof, wherein R₁, R₂, and R_{C} are as previously defined.

Another embodiment of the present invention is a method of inhibiting cleavage of amyloid precursor protein or mutant thereof at a site between amino acids, comprising administering a therapeutically effective amount of at least one compound of formula (I), or a pharmaceutically acceptable salt thereof, wherein R₁, R₂, and R_{C} are as previously defined, and wherein the site between amino acids corresponds to between Met652 and Asp653 (numbered for the APP-751 isotype), between Met671 and Asp672 (numbered for the APP-770 isotype), between Leu596 and Asp597 of the APP-695 Swedish Mutation, between Leu652 and Asp653 of the APP-751 Swedish Mutation, or between Leu671 and Asp672 of the APP-770 Swedish Mutation.

Another embodiment of the present invention is a method of inhibiting production of A-beta, comprising administering to a patient a therapeutically effective amount of at least one compound of formula (I), or a pharmaceutically acceptable salt thereof, wherein R₁, R₂, and R_{C} are as previously defined.

Another embodiment of the present invention is a method of preventing or treating deposition of A-beta, comprising administering a therapeutically effective amount of at least one compound of formula (I), or a pharmaceutically acceptable salt thereof, wherein R₁, R₂, and R_{C} are as previously defined.

Another embodiment of the present invention is a method of preventing, delaying, halting, or reversing a disease characterized by A-beta deposits or plaques, comprising administering a therapeutically effective amount of at least one compound of formula (I), or a pharmaceutically acceptable salt thereof, wherein R₁, R₂, and R_{C} are as previously defined.

In an embodiment, the A-beta deposits or plaques are in a human brain.

Another embodiment of the present invention is a method of preventing, delaying, halting, or reversing a condition associated with a pathological form of A-beta in a host comprising administering to a patient in need thereof an effective amount of at least one compound of formula (I), or a pharmaceutically acceptable salt thereof, wherein R₁, R₂, and R_{C} are as previously defined.

Another embodiment of the present invention is a method of inhibiting the activity of at least one aspartyl protease in a patient in need thereof, comprising administering a therapeutically effective amount of at least one compound of formula (I), or a pharmaceutically acceptable salt thereof to the patient, wherein R₁, R₂, and R_{C} are as previously defined.

In an embodiment, the at least one aspartyl protease is beta-secretase.

Another embodiment of the present invention is a method of interacting an inhibitor with beta-secretase, comprising administering to a patient in need thereof a therapeutically effective amount of at least one compound of formula (I), or a pharmaceutically acceptable salt thereof, wherein R₁, R₂, and R_{C} are as previously defined, and wherein the at least one compound interacts with at least one beta-secretase subsite such as S, S1′, or S2′.

Another embodiment of the present invention is a method of selecting compounds of formula (I) wherein the pharmacokinetic parameters are adjusted for an increase in desired effect (e.g., increased brain uptake).

Another embodiment is a method of selecting compounds of formula (I) wherein Cₘₐₓ, Tₘₐₓ, and/or half-life are adjusted to provide for maximum efficacy.

Another embodiment of the present invention is a method of treating a condition in a patient, comprising administering a therapeutically effective amount of at least one compound of formula (I), or a pharmaceutically acceptable salt, derivative or biologically active metabolite thereof, to the patient, wherein R₁, R₂, and R_{C} are as previously defined.

In an embodiment, the condition is Alzheimer's disease.

In another embodiment, the condition is dementia.

In another embodiment of the present invention, the compounds of formula (I) are administered in oral dosage form. The oral dosage forms are generally administered to the patient 1, 2, 3, or 4 times daily. It is preferred that the compounds be administered either three or fewer times daily, more preferably once or twice daily. It is preferred that, whatever oral dosage form is used, it be designed so as to protect the compounds from the acidic environment of the stomach. Enteric coated tablets are well known to those skilled in the art. In addition, capsules filled with small spheres, each coated to be protected from the acidic stomach, are also well known to those skilled in the art.

Therapeutically effective amounts include, for example, oral administration from about 0.1 mg/day to about 1,000 mg/day, parenteral, sublingual, intranasal, intrathecal administration from about 0.2 to about 100 mg/day, depot administration and implants from about 0.5 mg/day to about 50 mg/day, topical administration from about 0.5 mg/day to about 200 mg/day, and rectal administration from about 0.5 mg/day to about 500 mg/day.

When administered orally, an administered amount therapeutically effective to inhibit beta-secretase activity, to inhibit A-beta production, to inhibit A-beta deposition, or to treat or prevent Alzheimer's disease is from about 0.1 mg/day to about 1,000 mg/day.

In various embodiments, the therapeutically effective amount may be administered in, for example, pill, tablet, capsule, powder, gel, or elixir form, and/or combinations thereof. It is understood that, while a patient may be started at one dose or method of administration, that dose or method of administration may vary over time as the patient's condition changes.

Another embodiment of the present invention is a method of prescribing a medication for preventing, delaying, halting, or reversing disorders, conditions or diseases associated with amyloidosis. The method includes identifying in a patient symptoms associated with disorders, conditions or diseases associated with amyloidosis, and prescribing at least one dosage form of at least one compound of formula (I), or a pharmaceutically acceptable salt, to the patient, wherein R₁, R₂, and R_{C} are as previously defined.

Another embodiment of the present invention is an article of manufacture, comprising (a) at least one dosage form of at least one compound of formula (I), or a pharmaceutically acceptable salt thereof, wherein R₁, R₂, and R_{C} are as previously defined, (b) a package insert providing that a dosage form comprising a compound of formula (I) should be administered to a patient in need of therapy for at least one disorder, condition or disease associated with amyloidosis, and (c) at least one container in which at least one dosage form of at least one compound of formula (I) is stored.

Another embodiment of the present invention is a packaged pharmaceutical composition for treating conditions related to amyloidosis, comprising (a) a container which holds an effective amount of at least one compound of formula (I), or a pharmaceutically acceptable salt thereof, wherein R₁, R₂, and R_{C} are as previously defined, and (b) instructions for using the pharmaceutical composition.

Another embodiment of the present invention is an article of manufacture, comprising (a) a therapeutically effective amount of at least one compound of formula (I), or pharmaceutically acceptable salt thereof, wherein R₁, R₂, and R_{C} are as previously defined, (b) a package insert providing an oral dosage form should be administered to a patient in need of therapy for at least one disorder, condition or disease associated with amyloidosis, and (c) at least one container comprising at least one oral dosage form of at least one compound of formula (I).

Another embodiment of the present invention is an article of manufacture, comprising (a) at least one oral dosage form of at least one compound of formula (I), or a pharmaceutically acceptable salt thereof, wherein R₁, R₂, and R_{C} are as previously defined, in a dosage amount ranging from about 2 mg to about 1000 mg, associated with (b) a package insert providing that an oral dosage form comprising a compound of formula (I) in a dosage amount ranging from about 2 mg to about 1000 mg should be administered to a patient in need of therapy for at least one disorder, condition or disease associated with amyloidosis, and (c) at least one container in which at least one oral dosage form of at least one compound of formula (I) in a dosage amount ranging from about 2 mg to about 1000 mg is stored.

Another embodiment of the present invention is an article of manufacture, comprising (a) at least one oral dosage form of at least one compound of formula (I) in a dosage amount ranging from about 2 mg to about 1000 mg in combination with (b) at least one therapeutically active agent, associated with (c) a package insert providing that an oral dosage form comprising a compound of formula (I) in a dosage amount ranging from about 2 mg to about 1000 mg in combination with at least one therapeutically active agent should be administered to a patient in need of therapy for at least one disorder, condition or disease associated with amyloidosis, and (d) at least one container in which at least one dosage form of at least one compound of formula (I) in a dosage amount ranging from about 2 mg to about 1000 mg in combination with a therapeutically active agent is stored.

Another embodiment of the present invention is an article of manufacture, comprising (a) at least one parenteral dosage form of at least one compound of formula (I) in a dosage amount ranging from about 0.2 mg/mL to about 50 mg/mL, associated with (b) a package insert providing that a parenteral dosage form comprising a compound of formula (I) in a dosage amount ranging from about 0.2 mg/mL to about 50 mg/mL should be administered to a patient in need of therapy for at least one disorder, condition or disease associated with amyloidosis, and (c) at least one container in which at least one parenteral dosage form of at least one compound of formula (I) in a dosage amount ranging from about 0.2 mg/mL to about 50 mg/mL is stored.

Another embodiment of the present invention is an article of manufacture comprising (a) a medicament comprising an effective amount of at least one compound of formula (I) in combination with active and/or inactive pharmaceutical agents, (b) a package insert providing that an effective amount of at least one compound of formula (I) should be administered to a patient in need of therapy for at least one disorder, condition or disease associated with amyloidosis, and (c) a container in which a medicament comprising an effective amount of at least one compound of formula (I) in combination with therapeutically active and/or inactive agents is stored.

In an embodiment, the therapeutically active agent is selected from an antioxidant, an anti-inflammatory, a gamma-secretase inhibitor, a neurotropic agent, an acetyl cholinesterase inhibitor, a statin, an A-beta, and/or an anti-A-beta antibody.

Another embodiment is a kit comprising at least one component independently selected from: (a) at least one dosage form of a formula (I) compound; (b) at least one container in which at least one dosage form of a formula (I) compound is stored; (c) a package insert (optionally containing information of the dosage amount and duration of exposure of a dosage form containing at least one compound of formula (I) and optionally providing that the dosage form should be administered to a patient in need of therapy for at least one disorder, condition or disease associated with amyloidosis; and (d) at least one therapeutically active agent (optionally selected from an antioxidant, an anti-inflammatory, a gamma-secretase inhibitor, a neurotrophic agent, an acetyl cholinesterase inhibitor, a statin, an A-beta or fragment thereof, and an anti-A-beta antibody).

Another embodiment of the present invention is a method of producing a beta-secretase complex comprising exposing beta-secretase to a compound of formula (I), wherein R₁, R₂, and R_{C} are as previously defined, or a pharmaceutically acceptable salt thereof, in a reaction mixture under conditions suitable for the production of the complex.

Another embodiment of the present invention is a manufacture of a medicament for preventing, delaying, halting, or reversing Alzheimer's disease, comprising adding an effective amount of at least one compound of formula (I) to a pharmaceutically acceptable carrier.

Another embodiment of the present invention is a method of selecting a beta-secretase inhibitor comprising targeting at least one moiety of a formula (I) compound, or a pharmaceutically acceptable salt thereof, to interact with at least one beta-secretase subsite such as, but not limited to, S1, S1′, or S2′.

The methods of treatment described herein include administering the compounds of formula (I) orally, parenterally (via intravenous injection (IV), intramuscular injection (IM), depo-IM, subcutaneous injection (SC or SQ), or depo-SQ), sublingually, intranasally (inhalation), intrathecally, topically, or rectally. Dosage forms known to those skilled in the art are suitable for delivery of the compounds of formula (I).

In treating or preventing the above diseases, the compounds of formula (I) are administered using a therapeutically effective amount. The therapeutically effective amount will vary depending on the particular compound used and the route of administration, as is known to those skilled in the art.

The compositions are preferably formulated as suitable pharmaceutical preparations, such as for example, pill, tablet, capsule, powder, gel, or elixir form, and/or combinations thereof, for oral administration or in sterile solutions or suspensions for parenteral administration. Typically the compounds described above are formulated into pharmaceutical compositions using techniques and/or procedures well known in the art.

For example, a therapeutically effective amount of a compound or mixture of compounds of formula (I), or a physiologically acceptable salt is combined with a physiologically acceptable vehicle, carrier, binder, preservative, stabilizer, flavor, and the like, in a unit dosage form as called for by accepted pharmaceutical practice and as defined herein. The amount of active substance in those compositions or preparations is such that a suitable dosage in the range indicated is obtained. The compound concentration is effective for delivery of an amount upon administration that lessens or ameliorates at least one symptom of the disorder for which the compound is administered. For example, the compositions can be formulated in a unit dosage form, each dosage containing from about 2 mg to about 1000 mg.

The active ingredient may be administered in a single dose, or may be divided into a number of smaller doses to be administered at intervals of time. It is understood that the precise dosage and duration of treatment is a function of the disease or condition being treated and may be determined empirically using known testing protocols or by extrapolation from in vivo or in vitro test data. Also, concentrations and dosage values may vary with the severity of the condition to be alleviated. It is also to be understood that the precise dosage and treatment regimens may be adjusted over time according to the individual need and the professional judgment of the person administering or supervising the administration of the compositions, and that the concentration ranges set forth herein are exemplary only and are not intended to limit the scope or practice of the claimed compositions. A dosage and/or treatment method for any particular patient also may depend on, for example, the age, weight, sex, diet, and/or health of the patient, the time of administration, and/or any relevant drug combinations or interactions.

To prepare compositions to be employed in the methods of treatment, at least one compound of formula (I) is mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. An effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. Additionally, the active materials can also be mixed with other active materials that do not impair the desired action, or with materials that supplement the desired action, or have another action. For example, the compounds of formula (I) may be formulated as the sole pharmaceutically active ingredient in the composition or may be combined with other active ingredients.

Where the compounds exhibit insufficient solubility, methods for solubilizing may be used. Such methods are known and include, for example, using co-solvents (such as dimethylsulfoxide, (DMSO)), using surfactants (such as Tween®), and/or dissolution in aqueous sodium bicarbonate. Derivatives of the compounds, such as salts, metabolites, and/or pro-drugs, may also be used in formulating effective pharmaceutical compositions. Such derivatives may improve the pharmacokinetic properties of treatment administered.

The compounds of formula (I) may be prepared with carriers that protect them against rapid elimination from the body, such as time-release formulations or coatings. Such carriers include controlled release formulations, such as, for example, microencapsulated delivery systems and the like. The active compound is included in the pharmaceutically acceptable carrier in an amount sufficient to exert a therapeutically useful effect in the absence of undesirable side effects on the patient treated. Alternatively, the active compound is included in an amount sufficient to exert a therapeutically useful effect and/or minimize the severity and form of undesirable side effects. The therapeutically effective concentration may be determined empirically by testing the compounds in known in vitro and/or in vivo model systems for the treated disorder.

The tablets, pills, capsules, troches, and the like may contain a binder (e.g., gum tragacanth, acacia, corn starch, gelatin, and the like); a vehicle (e.g., microcrystalline cellulose, starch, lactose, and the like); a disintegrating agent (e.g., alginic acid, corn starch, and the like); a lubricant (e.g., magnesium stearate and the like); a gildant (e.g., colloidal silicon dioxide and the like); a sweetening agent (e.g., sucrose, saccharin, and the like); a flavoring agent (e.g., peppermint, methyl salicylate, fruit flavoring, and the like); compounds of a similar nature, and/or mixtures thereof.

When the dosage unit form is a capsule, it can contain, in addition to material described above, a liquid carrier such as a fatty oil. Additionally, dosage unit forms can contain various other materials, which modify the physical form of the dosage unit, for example, coatings of sugar or other enteric agents. A method of treatment can also administer the compound as a component of an elixir, suspension, syrup, wafer, chewing gum or the like. A syrup may contain, in addition to the active compounds, sucrose as a sweetening agent, flavors, preservatives, dyes and/or colorings.

The methods of treatment may employ at least one carrier that protects the compound against rapid elimination from the body, such as time-release formulations or coatings. Such carriers include controlled release formulations, such as, for example, implants or microencapsulated delivery systems, or biodegradable, biocompatible polymers such as collagen, ethylene vinyl acetate, polyanhydrides, polyglycolic acid, polyorthoesters, polylactic acid, and the like. Methods for preparation of such formulations are known to those in the art.

When orally administered, the compounds of the present invention can be administered in usual dosage forms for oral administration as is well known to those skilled in the art. These dosage forms include the usual solid unit dosage forms of tablets and capsules as well as liquid dosage forms such as solutions, suspensions, and elixirs. When solid dosage forms are used, it is preferred that they be of the sustained release type so that the compounds of the present invention need to be administered only once or twice daily. When liquid oral dosage forms are used, it is preferred that they be of about 10 mL to about 30 mL each. Multiple doses may be administered daily.

The methods of treatment may also employ a mixture of the active materials and other active or inactive materials that do not impair the desired action, or with materials that supplement the desired action.

Solutions or suspensions used for parenteral, intradermal, subcutaneous, or topical application can include a sterile diluent (e.g., water for injection, saline solution, fixed oil, and the like); a naturally occurring vegetable oil (e.g., sesame oil, coconut oil, peanut oil, cottonseed oil, and the like); a synthetic fatty vehicle (e.g., ethyl oleate, polyethylene glycol, glycerine, propylene glycol, and the like, including other synthetic solvents); antimicrobial agents (e.g., benzyl alcohol, methyl parabens, and the like); antioxidants (e.g., ascorbic acid, sodium bisulfite, and the like); chelating agents (e.g., ethylenediaminetetraacetic acid (EDTA), and the like); buffers (e.g., acetates, citrates, phosphates, and the like); and/or agents for the adjustment of tonicity (e.g., sodium chloride, dextrose, and the like); or mixtures thereof.

Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and the like, and mixtures thereof. Liposomal suspensions including tissue-targeted liposomes may also be suitable as pharmaceutically acceptable carriers. These may be prepared according to methods known, for example, as described in U.S. Pat. No. 4,522,811.

The methods of treatment include delivery of the compounds of the present invention in a nano crystal dispersion formulation. Preparation of such formulations is described, for example, in U.S. Pat. No. 5,145,684. Nano crystalline dispersions of Human Immunodeficiency Viral (HIV) protease inhibitors and their method of use are described in U.S. Pat. No. 6,045,829. The nano crystalline formulations typically afford greater bioavailability of drug compounds.

The methods of treatment include administration of the compounds parenterally, for example, by IV, IM, SC, or depo-SC. When administered parenterally, a therapeutically effective amount of about 0.2 mg/mL to about 50 mg/mL is preferred. When a depot or IM formulation is used for injection once a month or once every two weeks, the preferred dose should be about 0.2 mg/mL to about 50 mg/mL.

The methods of treatment include administration of the compounds sublingually. When given sublingually, the compounds of the present invention should be given one to four times daily in the amounts described above for IM administration.

The methods of treatment include administration of the compounds intranasally. When given by this route, the appropriate dosage forms are a nasal spray or dry powder, as is known to those skilled in the art. The dosage of the compounds of the present invention for intranasal administration is the amount described above for IM administration.

The methods of treatment include administration of the compounds intrathecally. When given by this route the appropriate dosage form can be a parenteral dosage form as is known to those skilled in the art. The dosage of the compounds of the present invention for intrathecal administration is the amount described above for IM administration.

The methods of treatment include administration of the compounds topically. When given by this route, the appropriate dosage form is a cream, ointment, or patch. When topically administered, the dosage is from about 0.2 mg/day to about 200 mg/day. Because the amount that can be delivered by a patch is limited, two or more patches may be used. The number and size of the patch is not important. What is important is that a therapeutically effective amount of a compound of the present invention be delivered as is known to those skilled in the art. The compound can be administered rectally by suppository as is known to those skilled in the art. When administered by suppository, the therapeutically effective amount is from about 0.2 mg to about 500 mg.

The methods of treatment include administration of the compounds by implants as is known to those skilled in the art. When administering a compound of the present invention by implant, the therapeutically effective amount is the amount described above for depot administration.

Given a particular compound of the present invention and/or a desired dosage form and medium, one skilled in the art would know how to prepare and administer the appropriate dosage form and/or amount.

The methods of treatment include use of the compounds of the present invention, or acceptable pharmaceutical salts thereof, in combination, with each other or with other therapeutic agents, to treat or prevent the conditions listed above. Such agents or approaches include acetylcholinesterase inhibitors such as tacrine (tetrahydroaminoacridine, marketed as COGNEX®), donepezil hydrochloride, (marketed as Aricept®) and rivastigmine (marketed as Exelon®); gamma-secretase inhibitors; anti-inflammatory agents such as cyclooxygenase II inhibitors; anti-oxidants such as Vitamin E or ginkolides; immunological approaches, such as, for example, immunization with A-beta peptide or administration of anti-A-beta peptide antibodies; statins; and direct or indirect neurotropic agents such as Cerebrolysin®, AIT-082 (Emilien, 2000, Arch. Neurol. 57:454), and other neurotropic agents; and complexes with beta-secretase or fragments thereof.

Additionally, the methods of treatment also employ the compounds of the present invention with inhibitors of P-glycoprotein (P-gp). P-gp inhibitors and the use of such compounds are known to those skilled in the art. See, for example, Cancer Research, 53, 4595-4602 (1993), Clin. Cancer Res., 2, 7-12 (1996), Cancer Research, 56, 4171-4179 (1996), International Publications WO 99/64001 and WO 01/10387. The blood level of the P-gp inhibitor should be such that it exerts its effect in inhibiting P-gp from decreasing brain blood levels of the compounds of formula (I). To that end the P-gp inhibitor and the compounds of formula (I) can be administered at the same time, by the same or different route of administration, or at different times. Given a particular compound of formula (I), one skilled in the art would know whether a P-gp inhibitor is desirable for use in the method of treatment, which P-gp inhibitor should be used, and how to prepare and administer the appropriate dosage form and/or amount.

Suitable P-gp inhibitors include cyclosporin A, verapamil, tamoxifen, quinidine, Vitamin E-TGPS, ritonavir, megestrol acetate, progesterone, rapamycin, 10,11-methanodibenzosuberane, phenothiazines, acridine derivatives such as GF120918, FK506, VX-710, LY335979, PSC-833, GF-102,918, quinoline-3-carboxylic acid (2-{4-[2-(6,7-dimethyl-3,4-dihydro-1H-isoquinoline-2-yl)-ethyl]phenylcarbamoyl}-4,5-dimethylphenyl)-amide (Xenova), or other compounds. Compounds that have the same function and therefore achieve the same outcome are also considered to be useful.

The P-gp inhibitors can be administered orally, parenterally, (via IV, IM, depo-IM, SQ, depo-SQ), topically, sublingually, rectally, intranasally, intrathecally, or by implant.

The therapeutically effective amount of the P-gp inhibitors is from about 0.1 mg/kg to about 300 mg/kg daily, preferably about 0.1 mg/kg to about 150 mg/kg daily. It is understood that while a patient may be started on one dose, that dose may vary over time as the patient's condition changes.

When administered orally, the P-gp inhibitors can be administered in usual dosage forms for oral administration as is known to those skilled in the art. These dosage forms include the usual solid unit dosage forms of tablets or capsules as well as liquid dosage forms such as solutions, suspensions or elixirs. When the solid dosage forms are used, it is preferred that they be of the sustained release type so that the P-gp inhibitors need to be administered only once or twice daily. The oral dosage forms are administered to the patient one through four times daily. It is preferred that the P-gp inhibitors be administered either three or fewer times a day, more preferably once or twice daily. Hence, it is preferred that the P-gp inhibitors be administered in solid dosage form and further it is preferred that the solid dosage form be a sustained release form which permits once or twice daily dosing. It is preferred that the dosage form used is designed to protect the P-gp inhibitors from the acidic environment of the stomach. Enteric coated tablets are well known to those skilled in the art. In addition, capsules filled with small spheres each coated to protect from the acidic stomach, are also well known to those skilled in the art.

In addition, the P-gp inhibitors can be administered parenterally. When administered parenterally they can be administered via IV, IM, depo-IM, SQ or depo-SQ.

The P-gp inhibitors can be given sublingually. When given sublingually, the P-gp inhibitors should be given one through four times daily in the same amount as for IM administration.

The P-gp inhibitors can be given intranasally. When given by this route of administration, the appropriate dosage forms are a nasal spray or dry powder as is known to those skilled in the art. The dosage of the P-gp inhibitors for intranasal administration is the same as for IM administration.

The P-gp inhibitors can be given intrathecally. When given by this route of administration the appropriate dosage form can be a parenteral dosage form as is known to those skilled in the art.

The P-gp inhibitors can be given topically. When given by this route of administration, the appropriate dosage form is a cream, ointment or patch. Because of the amount of the P-gp inhibitors needed to be administered the patch is preferred. However, the amount that can be delivered by a patch is limited. Therefore, two or more patches may be required. The number and size of the patch is not important; what is important is that a therapeutically effective amount of the P-gp inhibitors be delivered as is known to those skilled in the art.

The P-gp inhibitors can be administered rectally by suppository or by implants, both of which are known to those skilled in the art.

It should be apparent to one skilled in the art that the exact dosage and frequency of administration will depend on the particular compounds of the present invention administered, the particular condition being treated, the severity of the condition being treated, the age, weight, or general physical condition of the particular patient, or any other medication the individual may be taking as is well known to administering physicians who are skilled in this art.

### EXPERIMENTAL PROCEDURES

The compounds and the methods of treatment of the present invention can be prepared by one skilled in the art based on knowledge of the compound's chemical structure. The chemistry for the preparation of compounds employed in the methods of treatment of this invention is known to those skilled in the art. In fact, there is more than one process to prepare the compounds employed in the methods of treatment of the present invention. Specific examples of methods of preparing the compounds of the present invention can be found in the art. For examples, see Zuccarello et al., J. Org. Chem. 1998, 63, 4898-4906; Benedetti et al., J. Org. Chem. 1997, 62, 9348-9353; Kang et al., J. Org. Chem. 1996, 61, 5528-5531; Kempf et al., J. Med. Chem. 1993, 36, 320-330; Lee et al., J. Am. Chem. Soc. 1999, 121, 1145-1155; and references cited therein; Chem. Pharm. Bull. (2000), 48(11), 1702-1710; J. Am. Chem. Soc. (1974), 96(8), 2463-72; Ind. J. Chem., §B: Organic Chemistry Including Medicinal Chemistry (2003), 42B(4), 910-915; and J. Chem. Soc. §C: Organic (1971), (9), 1658-10. See also U.S. Pat. Nos. 6,150,530, 5,892,052, 5,696,270, and 5,362,912, and references cited therein, which are incorporated herein by reference.

¹H and ¹³C NMR spectra were obtained on a Varian 400 MHz, Varian 300 MHz, or Bruker 300 MHz instrument. HPLC samples were analyzed using a YMC ODS-AQ S-3 120 A 3.0×50 mm cartridge, with a standard gradient from 5% acetonitrile containing 0.01% heptafluorobutyric acid (HFBA) and 1% isopropanol in water containing 0.01% HFBA to 95% acetonitrile containing 0.01% HFBA and 1% isopropanol in water containing 0.01% HFBA over 5 min. Mass spec samples were performed with electron spray ionization (ESI).

### EXAMPLE 1

### GENERAL SCHEME: PREPARATION OF REPRESENTATIVE COMPOUNDS OF FORMULA (I)

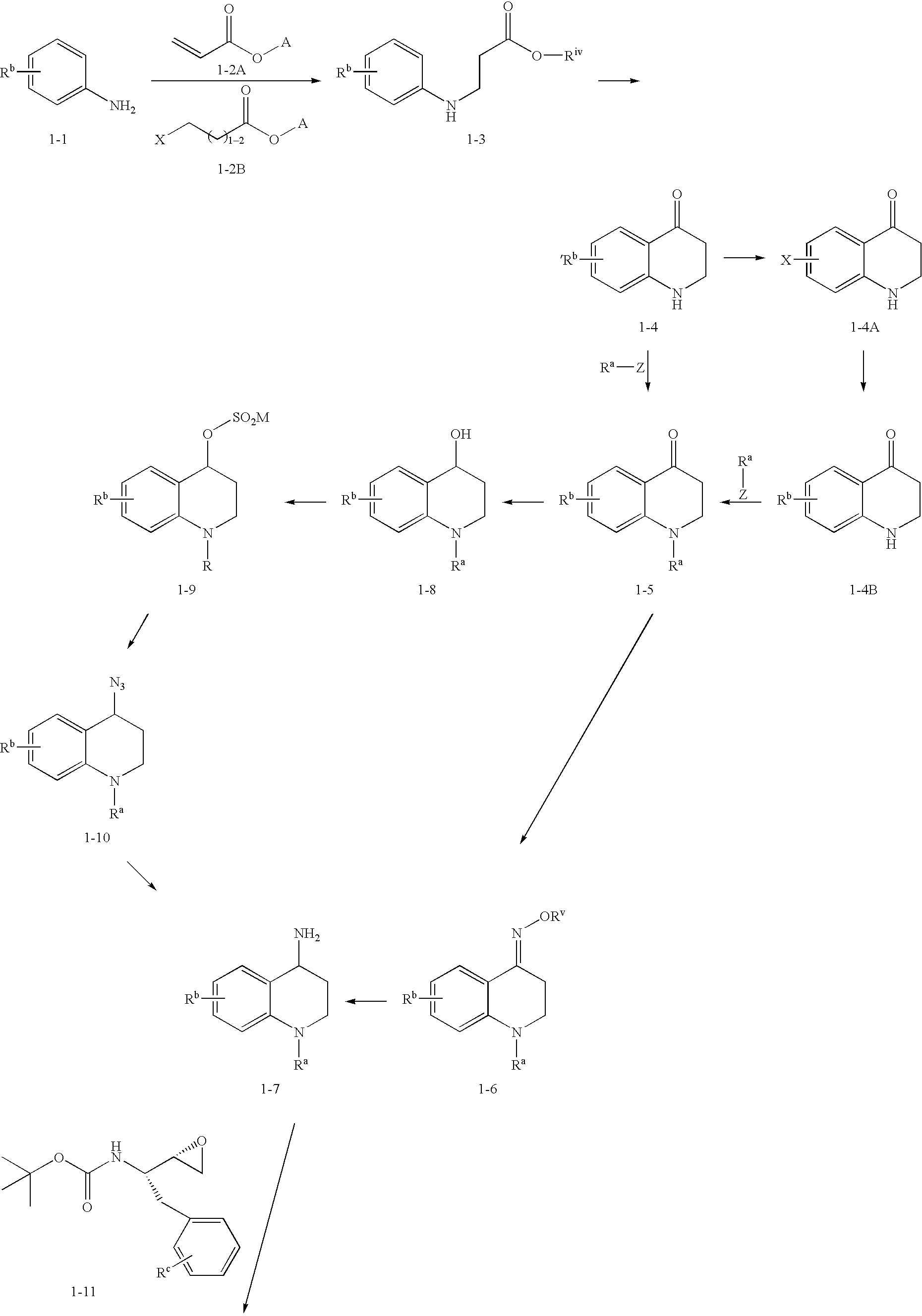

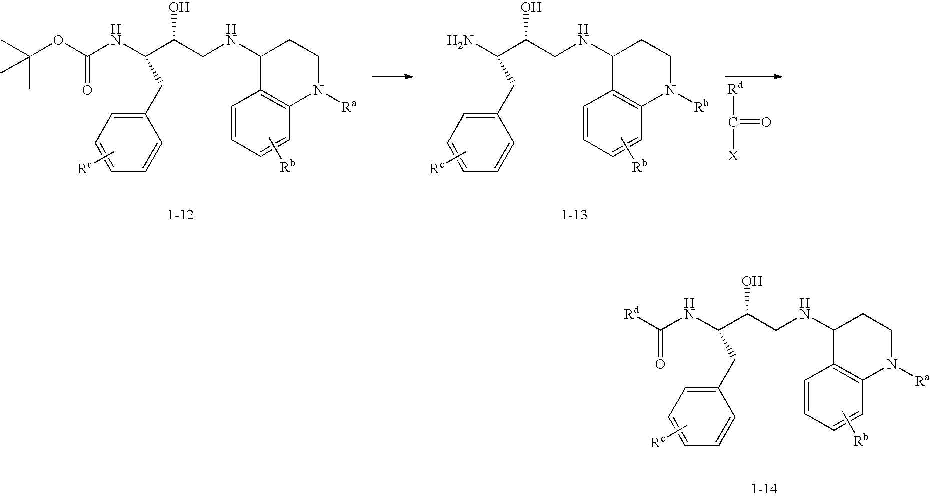

Aniline 1-1 is alkylated with a halide 1-2B or acrylate 1-2A to give 1-3. 1-3 is then treated with a strong acid or with a Lewis acid at temperatures ranging from 0° C. to 140° C., preferably with phosphorus pentoxide and methanesulfonic acid at 130° C., to give ketone 1-4. The nitrogen of 1-4 is then either protected with a protecting group, many of which are listed in Protective Groups in Organic Synthesis, Greene and Wuts, 3rd edition, 1999, Wiley-Interscience, or is substituted with an alkyl group, an acyl group, or a sulfonyl group. Protected ketone 1-5 may be prepared using R^{a}-Z via routes known in the art. Another alternative route of preparing 1-5 uses 1-4 with R^{b} as hydrogen. Halogenation with halogenating reagents such as N-bromosuccinimide, N-iodosuccinimide, dibromatin, and the like results in 1-4A where R^{b} is preferably bromine or iodine. Treatment of 1-4A under cross coupling conditions such as those described by Negishi (Tet. Lett. 1983, 3823), Huo (Org. Lett. 2003, 423) and reviewed by Knochel (Tetrahedron, 1998, 8275) provides 1-4B where R^{b} is alkyl. Further treatment of 1-4B with R^{a}-Z as described above gives 1-5.

The protected ketone 1-5 is then converted to amine 1-7 by several methods depending on the nature of the R^{a} group. In one method, 1-5 is treated with a hydroxyl amine in the presence of a base and a catalytic amount of acid in solvents such as methanol, ethanol, butanol, and the like, at temperatures ranging from room temperature to the reflux temperature of the solvent, yielding oxime 1-6. 1-6 is then reduced to amine 1-7 using a suitable catalyst, preferably palladium, under a blanket of hydrogen at pressures ranging from atmospheric to 100 pounds per square inch. Solvents such as methanol, ethanol, or ethyl acetate may be used.

Alternatively, protected ketone 1-5 may be reduced to alcohol 1-8 using reducing agents known to those skilled in the art, such as sodium borohydride in methanol or ethanol, depending on the nature of the R^{a} group, at temperatures ranging from 0° C. to 100° C. Alcohol 1-8 is then converted to sulfonate ester 1-9 with reagents such as methanesulfonyl chloride or toluenesulfonyl chloride using methods known to those skilled in the art. The sulfonate ester is displaced with azide using, for example, sodium azide in solvents, such as dichloromethane and DMF, at temperatures ranging from room temperature to 120° C., yielding azide 1-10. Azide 1-10 is then reduced to amine 1-7 using, for example, trimethylphosphine in solvents, such as THF and the like, at temperatures between 0° C. and the reflux temperature of the solvent. The choice of reducing agent will depend on the nature of the R^{a} and R^{b} groups and can generally be found in references such as Smith and March, March's Advanced Organic Chemistry: Reactions, Mechanisms, and Structure, 5th ed., 2001, Wiley-Interscience.

Amine 1-7 is then stirred in the presence of epoxide 1-11 in preferably, but not limited to, alcoholic solvents, such as ethanol, isopropyl, tert-butyl, or n-butyl alcohol, at temperatures ranging from 50° C. to the reflux temperature of the solvent, to give Boc-amine 1-12. Boc-amine 1-12 is then treated with strong acid, such as trifluoroacetic acid, in non-reactive solvents such as dichloromethane or with dry HCl in solvents such as dialkyl ethers or alcoholic solvents at temperatures ranging from room temperature to 80° C. to give, after washing with base, triamine 1-13. Triamine 1-13 is acylated by means known to those skilled in the art, for example, condensation with a carboxylic acid using coupling agents such as EDC, DCC, HATU, or HBTU and the like. Preferred methods are acylation with acyl imidazole or acetylation with N,N-diacetylmethoxyamine to give 1-14.

### EXAMPLE 2

### PREPARATION OF N-{(1S,2R)1-(3,5-DIFLUOROBENZYL)-3-[(6-ETHYL-1,2,3,4-TETRAHYDROQUINOLIN-4-YL)AMINO]-2-HYDROXYPROPYL)ACETAMIDE

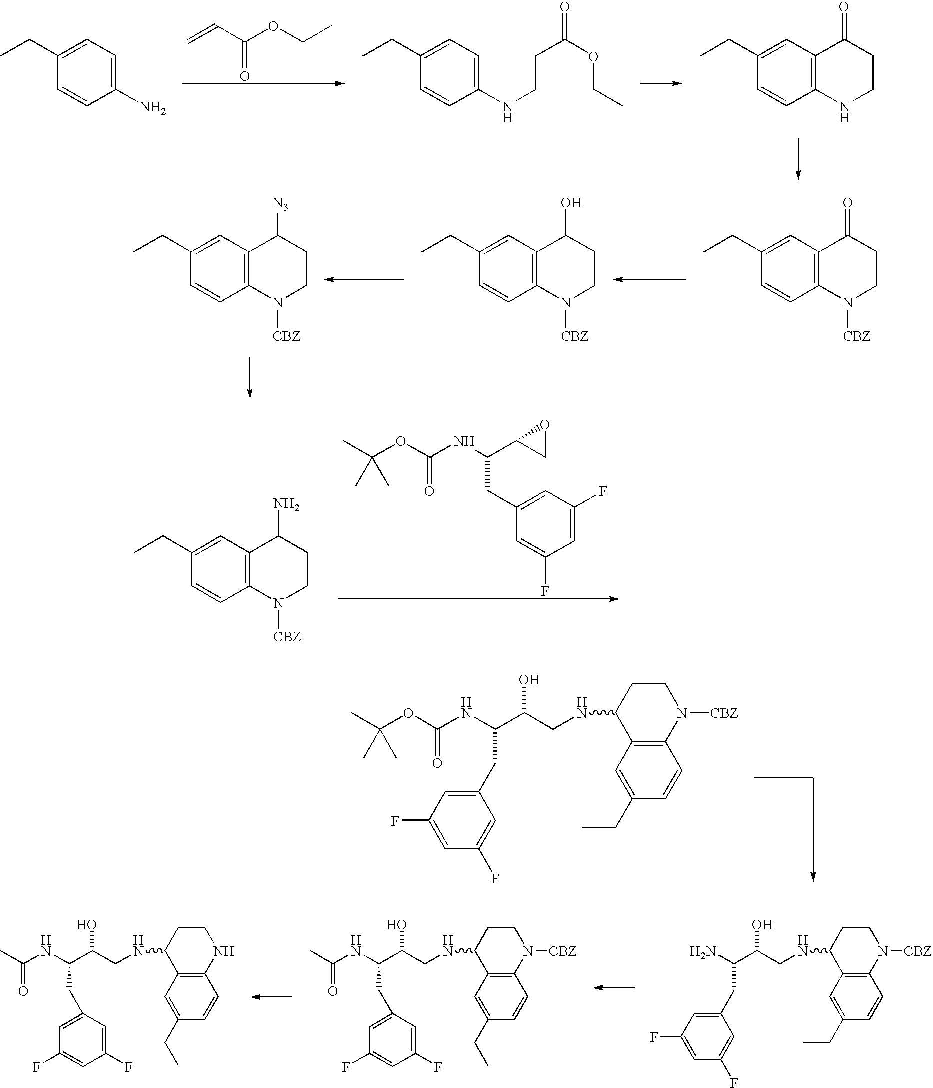

### Step 1. PREPARATION OF ETHYL N-(4-ETHYLPHENYL)-BETA-ALANINATE

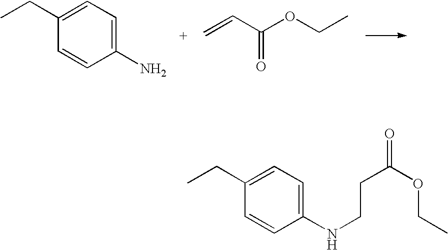

Ethyl acrylate (10.8 g) was added to a solution of 4-ethyl aniline (10.0 g) in acetic acid (25 mL). The mixture was heated to 80° C. for 2 h. Additional ethyl acrylate (1.0 mL) was added, and the mixture was again heated to 80° C. for 1 h. The mixture was allowed to cool to room temperature and stir for 2 days. Sodium hydroxide (8N) was added until the pH reached 9. The mixture was partitioned between dichloromethane and water and the combined organics were washed with 1 N sodium hydroxide, brine, dried (sodium sulfate), filtered, and concentrated. The mixture was chromatographed eluting with a 20:80 ethyl acetate:heptane solvent solution. A mixture of the mono and di-ester product (19.5 g) was obtained (1:1 mixture). MS (ESI+) for C₁₃H₁₉NO₂ m/z 221.99 [M+H]⁺.

### Step 2. PREPARATION OF 6-ETHYL-2,3-DIHYDROQUINOLIN-4(1H)-ONE

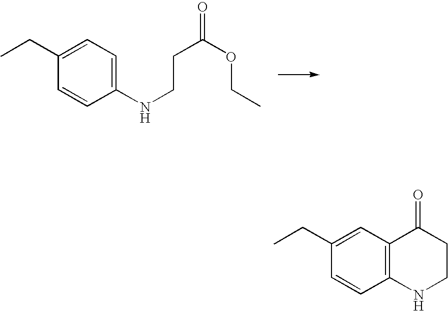

Phosphorus pentoxide (19.53 g) in methane sulfonic acid (200 mL) was heated to 130° C. and stirred for 1 h until all the solids had dissolved. The mixture was allowed to cool for 15 min and ethyl N-(4-ethylphenyl)-beta-alaninate (19.53 g of mono and di-ester mixture) was added. The mixture was heated to 130° C. for 1 h and allowed to slowly cool overnight. The mixture was then cooled in an ice bath and 10N sodium hydroxide was added until the pH reached 9.5. Ethyl acetate was added to the mixture to help dissolve solids. The remaining gummy dark solids were dissolved in methanol and added to the ethyl acetate-aq. sodium hydroxide mixture. Semi-crystalline solids precipitated and were removed by filtration through Celite®. The filtrate was washed with water, 1N sodium hydroxide, and brine, dried (magnesium sulfate), filtered, and concentrated. Silica gel chromatography using 0.25% ammonium hydroxide in dichloromethane yielded mixed fractions. The mixed fractions were combined and re-chromatographed using 30% ethyl acetate in heptane. The resulting material was further upgraded by formation of the hydrochloride salt using 2N HCl in ether. The salt was collected by filtration, washed with heptane and dried in an oven under vacuum at 50° C. overnight. The salt was then partitioned between dichloromethane and 1N sodium hydroxide. The organic layer was extracted twice with dichloromethane, washed with 1N sodium hydroxide, dried (sodium sulfate), filtered, and concentrated to give 3.83 g of the title compound. MS (ESI+) for C₁₁H₁₃NO m/z 175.96 [M+H]⁺.

### Step 3. PREPARATION OF BENZYL 6-ETHYL-4-OXO-3,4-DIHYDROQUINOLINE-1(2H)-CARBOXYLATE

Sodium bicarbonate (0.84 g) was added to a solution of 6-ethyl-2,3-dihydroquinolin-4(1H)-one (1.25 g) in THF (15 mL). Water (5 mL) followed by benzyl chloroformate (1.58 g) was added to the mixture, and it was stirred at room temperature overnight at which point additional NaHCO₃ (0.60 g) was added to the mixture and it was stirred at room temperature for an additional 2 h. The mixture was then concentrated under reduced pressure and the residue was partitioned between water and ethyl acetate. The organic layer was washed with brine, dried (magnesium sulfate), filtered, and concentrated. Chromatography on silica gel using 25% ethyl acetate in heptane solvent solution yielded 1.84 g of the title compound. MS (ESI+) for C₁₉H₁₉NO₃ m/z 310.03 [M+H]⁺.

### Step 4. PREPARATION OF BENZYL 6-ETHYL-4-HYDROXY-3,4-DIHYDROQUINOLINE-1(2H)-CARBOXYLATE

Benzyl 6-ethyl-4-hydroxy-3,4-dihydroquinoline-1(2H)-carboxylate was prepared essentially according to the procedure of preparing chroman-4-ol: NaBH₄ (5.5 g, 145 mmol) was added in 1 g portions to a MeOH (250 mL) solution of 4-chromanone (16.6 g, 11 mmol), at 0° C., over a 30 min period. The mixture was stirred for 1 h and allowed to warm to room temperature. The reaction was quenched with the slow addition of aq. NH₄Cl (100 mL). The MeOH was removed in vacuo and the residue extracted with Et₂O. The organic layers were dried (magnesium sulfate) and treated with activated carbon. After filtration, the Et₂O was removed in vacuo to yield 15.8 g of chroman-4-ol as a clear oil. HRMS (ESI+) calc'd for C₉H₁₀O₂ m/z 150.0681 [M+H]⁺; found 150.0679.

The crude product was purified by chromatography on silica gel using a 2% MeOH in dichloromethane solvent solution with 0.5% ammonium hydroxide. ¹H NMR (CDCl₃) δ 81.22 (t, J=8 Hz, 3 H), 1.89 (s, 1 H), 2.04 (m, 2 H), 2.61 (q, J=8 Hz, 2 H), 3.66 (m, 1 H), 4.11 (m, 1 H), 4.74 (t, J=4 Hz, 1 H), 5.25 (dd, J=12, 20 Hz, 2 H), 7.09 (dd, J=2, 9 Hz, 1 H), 7.21 (d, J=2 Hz, 1 H), 7.35 (m, 5 H), 7.6 (d, J=8 Hz, 1 H).

### Step 5. PREPARATION OF BENZYL 4-AMINO-6-ETHYL-3,4-DIHYDROQUINOLINE-1(2H)-CARBOXYLATE

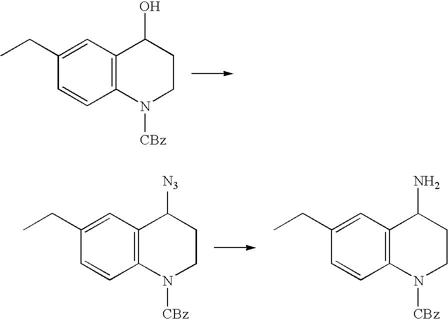

The above compound was prepared essentially according to the method of Example 50, step 2. First, the alcohol was converted to the azide. ¹H NMR (CDCl₃) δ 1.23 (t, J=8 Hz, 3 H), 2.09 (m, 2 H), 2.62 (q, J=8 Hz, 2 H), 3.67 (m, 1 H), 4.12 (m, 1 H), 4.58 (t, J=4 Hz, 1 H), 5.24 (m, 2 H), 7.09 (d, J=2 Hz, 1 H), 7.13 (dd, J=2, 9 Hz, 1 H), 7.35 (m, 5 H), 7.82 (d, J=8 Hz, 1 H).

Second, the azide was reduced using PMe₃ yielding benzyl 4-amino-6-ethyl-3,4-dihydroquinoline-1(2H)-carboxylate. MS (ESI+) for C₁₉H₂₂N₂O₂ m/z 311.05 [M+H]⁺.

### Step 6. PREPARATION OF BENZYL 4-{[(2R,3S)-3-[(TERT-BUTOXYCARBONYL)AMINO]-4-(3,5-DIFLUOROPHENYL)-2-HYDROXYBUTYL]AMINO}-3,4-DIHYDROQUINOLINE-1(2H)-CARBOXYLATE

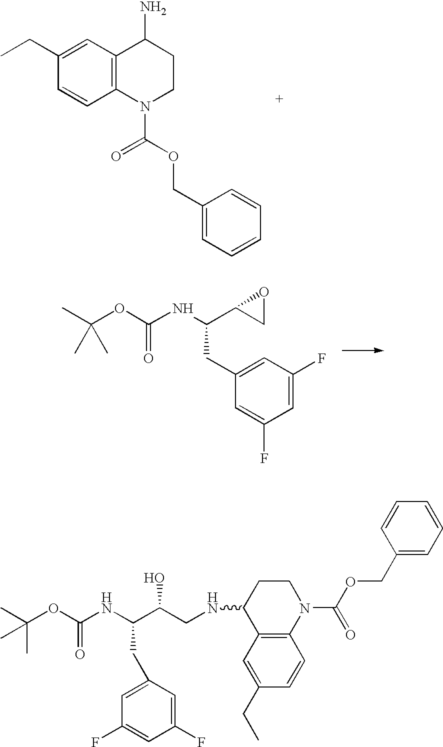

The above compound was prepared essentially according to the method of Example 50, Step 3. The crude product was purified by silica gel chromatography using 2% MeOH in dichloromethane with 0.25% NH₄OH as the solvent system. MS (ESI+) for C₃₄H₄₁F₂N₃O₅ m/z 610.51 [M+H]⁺.

### Step 7. PREPARATION OF BENZYL 4-{[(2R,3S)-3-AMINO-4-(3,5-DIFLUOROPHENYL)-2-HYDROXYBUTYL]AMINO}-6-ETHYL-3,4-DIHYDROQUINOLINE-1(2H)-CARBOXYLATE

2N HCl in Et₂O (1.6 mL) was added to a solution of the produce from Step 6 (0.76 g) in MeOH (10 mL). The mixture was stirred at room temperature for 2 h. Additional 2N HCl in Et₂O (1.0 mL) was added and stirred for 4 h. The, reaction was still not complete, so HCl in Et₂O (3.0 mL) was added and stirred for 2 h. The reaction was then stripped of solvent under reduced pressure. The residue was dissolved in ethyl acetate, washed with 1N NaOH, dried (magnesium sulfate), filtered, and concentrated. Silica gel chromatography (eluent: 4% MeOH in dichloromethane with 0.25% NH₄OH) yielded 0.44 g of the title compound. MS (ESI+) for C₂₉H₃₃F₂N₃O₃ m/z 510.36 [M+H]⁺.

### Step 8. PREPARATION OF BENZYL 4-{[(2R,3S)-3-(ACETYLAMINO)-4-(3,5-DIFLUOROPHENYL)-2-HYDROXYBUTYL]AMINO}-6-ETHYL-3,4-DIHYDROQUINOLINE-1(2H)-CARBOXYLATE

N,N-diacetyl-O-methylhydroxylamine (0.11 g) was added to a solution of the product from Step 7 (0.43 g) in dichloromethane (15 mL). Additional N,N-diacetyl-O-methylhydroxylamine (0.10 g) was added after stirring overnight at room temperature and again (0.10 g) after stirring for an additional 6 h. The mixture was then partitioned between dichloromethane, 1N HCl, and brine. The organic layer was dried (magnesium sulfate), filtered, and concentrated. A silica gel column was run for purification using 4% MeOH in dichloromethane with 0.25% NH₄OH as the solvent solution and yielded 0.35 g of the title compound. MS (ESI+) for C₃₁H₃₄F₂N₃O₄ m/z 552.32 [M+H]⁺.

### Step 9. PREPARATION OF N-{(1S,2R)-1-(3,5-DIFLUOROBENZYL)-3-[(6-ETHYL-1,2,3,4-TETRAHYDROQUINOLIN-4-YL)AMINO]-2-HYDROXYPROPYL}ACETAMIDE

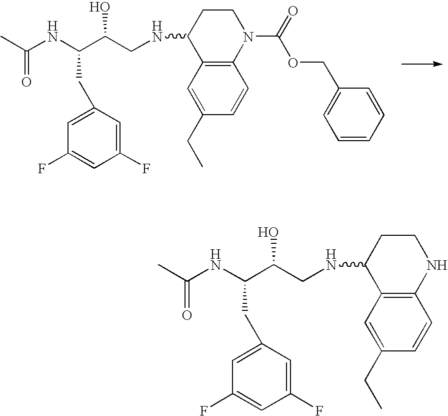

N₂(g) was bubbled through a solution of the product from Step 8 (0.35 g), EtOH (25 mL), and acetic acid (0.75 mL). 10% palladium on carbon (0.29 g) was added to the mixture and it was shaken on a hydrogenation apparatus under 52 psi of hydrogen for 1.25 h. The catalyst was filtered off using Celite® and the filtrate was concentrated under reduced pressure. The residue was partitioned between ethyl acetate, aq. sodium hydroxide (pH 10), and brine, and then dried (magnesium sulfate), filtered, and concentrated. Silica gel column chromatography (eluent: 6% MeOH in dichloromethane with 0.25% NH₄OH) gave 0.04 g of the title compound. MS (ESI+) for C₂₃H₂₉F₂N₃O₂ m/z 418.31 [M+H]⁺.

### EXAMPLE 3

### PREPARATION OF N-{(1S,2R)-1-(3,5-DIFLUOROBENZYL)-3-[(6-ETHYL-1-METHYL-1,2,3,4-TETRAHYDROQUINOLIN-4- YL)AMINO]-2-HYDROXYPROPYL}ACETAMIDE

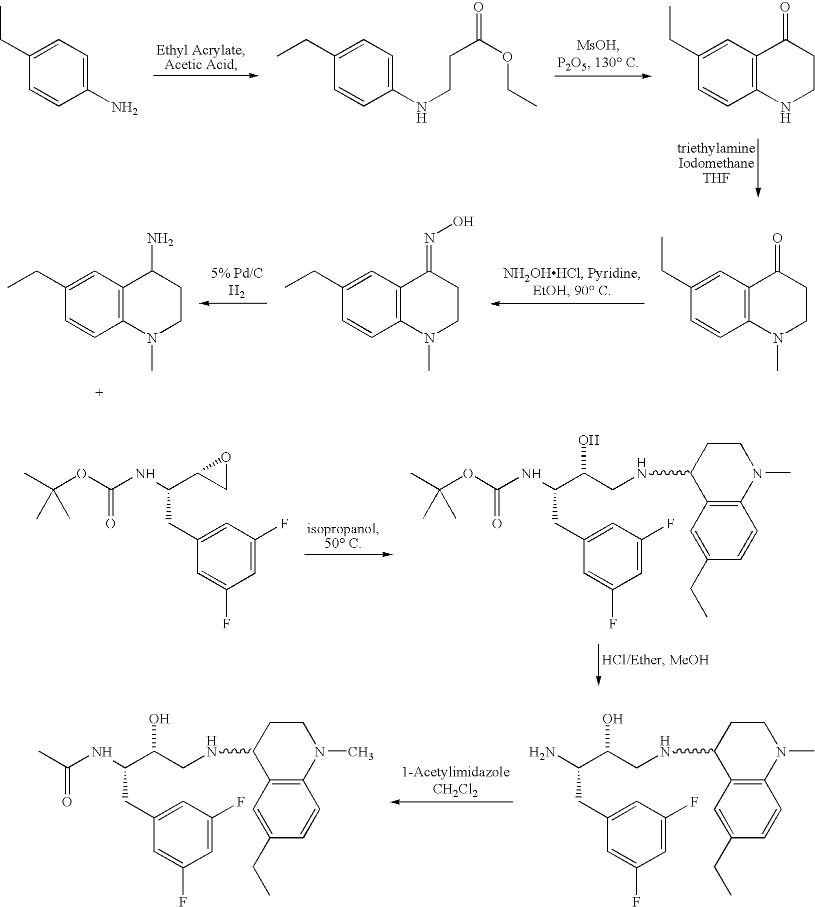

### Step 1. PREPARATION OF ETHYL N-(4-ETHYLPHENYL)-BETA-ALANINATE

Ethyl acrylate (8.26 g) was added to a solution of 4-ethyl aniline (10.00 g) in acetic acid (20 mL). The mixture was heated to 70° C. for 3.5 h. The mixture was allowed to cool to room temperature. The mixture was partitioned between dichloromethane and water. The combined organic extracts were washed with brine, dried (sodium sulfate), filtered, and concentrated. The product was used in the next step without further purification. MS (ESI+) for C₁₃H₁₉NO₂ m/z 223.1 [M+H]⁺.

### Step 2. PREPARATION OF 6-ETHYL-2,3-DIHYDROQUINOLIN-4(1H)-ONE

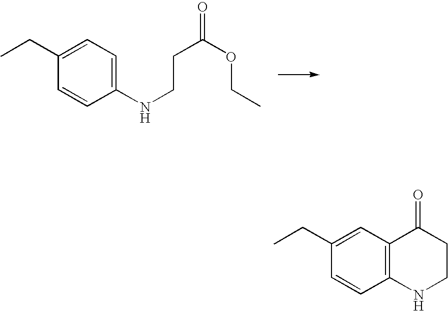

Phosphorus pentoxide (11.14 g) in methane sulfonic acid (114 mL) was stirred at 130° C. until it dissolved. The mixture was allowed to cool for 15 min, and ethyl N-(4-ethylphenyl)-beta-alaninate (11.14 g of mono and di-ester mixture) was added. The mixture was heated to 130° C. for 1.5 h, and the mixture was allowed to cool to room temperature. The mixture was cooled in an ice bath, and 50% sodium hydroxide was added until the pH reached 8. The gummy dark solids were dissolved in MeOH, and added to the mixture. Solids began to crash out, so they were filtered off with Celite®. The liquids were combined and partitioned between dichloromethane and water. The organics were extracted with dichloromethane, washed with brine, dried (sodium sulfate), filtered, and concentrated. The product was chromatographed using a 30% ethyl acetate in heptane solvent solution. 4.10 g of the title product were recovered. (28% yield through first two steps) MS (ESI+) for C₁₁ H₁₃NO m/z 176.00 [M+H]⁺.

### Step 3. PREPARATION OF 6-ETHYL-1-METHYL-2,3-DIHYDROQUINOLIN-4(1H)-ONE

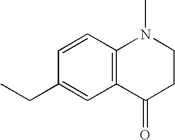

Triethylamine (0.64 g) followed by iodomethane (0.89 g) was added to a solution of 6-ethyl-2,3-dihydroquinolin-4(1H)-one (1.00 g) in THF (25 mL). The mixture was refluxed at 70° C. overnight. The solvent was stripped under reduced pressure, and the residue was partitioned between aqueous sodium bicarbonate and dichloromethane. The organics were extracted, washed with brine, dried (sodium sulfate), filtered, and concentrated. Chromatography (eluent: 40% ethyl acetate in heptane) yielded 0.32 g of the title product (30% yield). MS (ESI+) for C₁₂H₁₅NO m/z 190.10 [M+H]⁺.

### Step 4. PREPARATION OF (4E)-6-ETHYL-1-METHYL-2,3-DIHYDROQUINOLIN-4(1H)-ONE OXIME

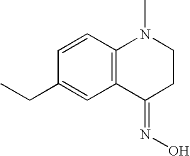

Pyridine (0.53) and hydroxylamine hydrochloride (0.59 g) were added to a solution of 6-ethyl-1-methyl-2,3-dihydroquinolin-4(1H)-one (0.32g) in ethanol (25 mL). The mixture was heated at 90° C. for 2 h with a reflux condensor attatched. The mixture was cooled to room temperature, and the solvent was stripped under reduced pressure. The residue was partitioned between water and dichloromethane, and the extracted organics were washed with brine, dried (sodium sulfate), filtered, and concentrated to yield (0.34 g) of the title product (98% yield). MS (ESI+) for C₁₂H₁₆N₂O m/z 205.02 [M+H]⁺.

### Step 5. PREPARATION OF 6-ETHYL-1-METHYL-1,2,3,4-TETRAHYDROQUINOLIN-4-AMINE

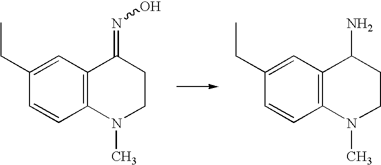

6-Ethyl-1-methyl-2,3-dihydroquinolin-4(1H)-one oxime (0.34 g), ethanol (20 mL), and acetic acid (0.27 g) were combined in a hydrogenation flask and degassed with N₂(g). 5% palladium on carbon was carefully added to the mixture (0.04 g) and the mixture was degassed for several more minutes. The mixture was set up on the hydrogenation apparatus and placed under 50 psi of hydrogen. The mixture was shaken for 5.5 h, but was not complete so the mixture was degassed, additional 5% palladium on carbon (0.10 g) was added, the mixture was put back on the hydrogenation apparatus, and was shaken overnight. The palladium on carbon was filtered off using Celite® and the liquids were concentrated under reduced pressure. The residue was partitioned between aqueous sodium bicarbonate and dichloromethane, extracted, and the extracted organics were dried (sodium sulfate), filtered, and concentrated to yield the title compound (0.26 g, 82% yield).

### Step 6. PREPARATION OF TERT-BUTYL (1 S,2R)-1-(3,5-DIFLUOROBENZYL)-3-[(6-ETHYL-1-METHYL-1,2,3,4-TETRAHYDROQUINOLIN-4-YL)AMINO]-2-HYDROXYPROPYLCARBAMATE

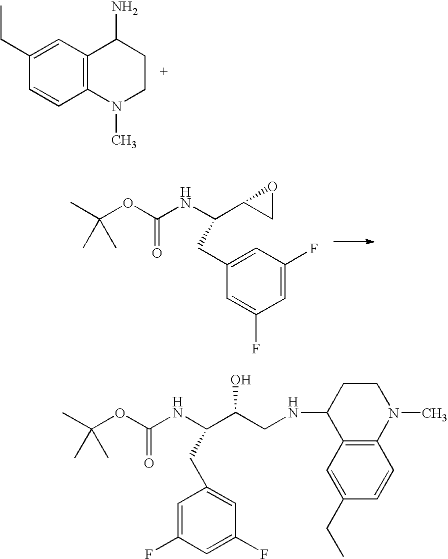

[1-(3,5-Difluoro-benzyl)-3-(6-ethyl-1-methyl-1,2,3,4-tetrahydro-quinolin-4-ylamino)-2-hydroxy-propyl]-carbamic acid tert-butyl ester was prepared essentially according to the method in Example 50, step 3, below: An isopropyl alcohol (25 mL) solution of tert-butyl (1S)-2-(3,5-difluorophenyl)-1-[(2S)-oxiran-2-yl]ethylcarbamate (2.2 g, 7.2 mmol) and 6-iodo-chroman-4-ylamine (3.0 g, 10.9 mmol) was stirred at 75° C. overnight. The IPA was removed in vacuo and the resulting residue was dissolved in EtOAc, washed with 1 N HCl, NaHCO₃, and brine, dried (sodium sulfate), and concentrated in vacuo to yield the title compound as a mixture of diastereomers. MS (ESI+) for C₂₇H₃₇F₂N₃O₃ m/z 490.59 [M+H]⁺.

### Step 7. PREPARATION OF (2R,3S)-3-AMINO-4-(3,5-DIFLUOROPHENYL)-1-[(6-ETHYL-1-METHYL-1,2,3,4-TETRAHYDROQUINOLIN-4-YL)AMINO]BUTAN-2-OL

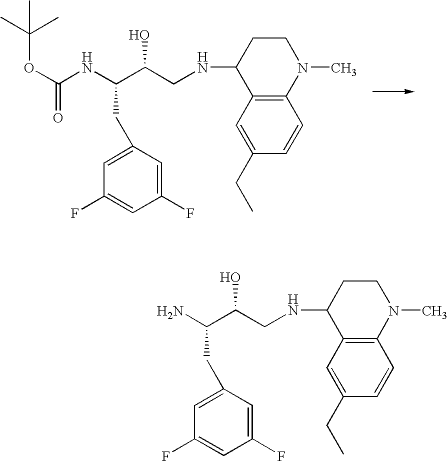

2N HCl in Et₂O (2.1 mL) was added to a solution of tert-butyl (1S,2R)-1-(3,5-difluorobenzyl)-3-[(6-ethyl-1-methyl-1,2,3,4-tetrahydroquinolin-4-yl)amino]-2-hydroxypropylcarbamate (0.412 g) in MeOH (5 mL). The mixture was stirred at room temperature for 15 min. The mixture was stripped of solvent under reduced pressure. The residue was partitioned between dichloromethane and aqueous sodium bicarbonate, and the organic layer was extracted, washed with brine, dried (sodium sulfate), filtered, and concentrated. Silica gel column chromatography (eluent: 5% MeOH in dichloromethane) yielded 0.255 g of the title product (78% yield). MS (ESI+) for C₂₂H₂₉F₂N₃O m/z 390.18 [M+H]⁺.

### Step 8. PREPARATION OF N-{1-(3,5-DIFLUOROBENZYL)-3-[(6-ETHYL-1-METHYL-1,2,3,4-TETRAHYDROQUINOLIN-4-YL)AMINO]-2-HYDROXYPROPYL}ACETAMIDE

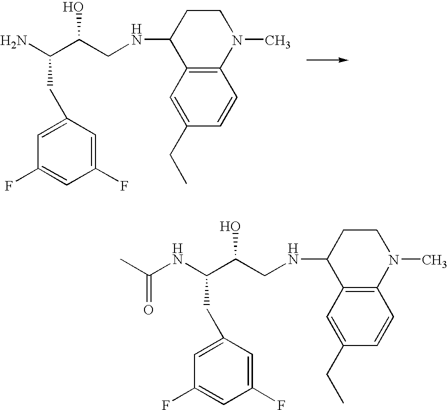

1-Acetylimidazole (0.062 g) was added to a solution of (2R,3S)-3-amino-4-(3,5-difluorophenyl)-1-[(6-ethyl-1-methyl-1,2,3,4-tetrahydroquinolin-4-yl)amino]butan-2-ol (0.218 g) in dichloromethane (15 mL). The mixture was stirred overnight at room temperature. The mixture was partitioned between dichloromethane and brine, and the organic layer was extracted, dried (sodium sulfate), filtered, and concentrated. Silica gel column chromatography (eluent: 3% MeOH in dichloromethane with 0.5% NH₄OH) yielded an impure solid, which was washed with 1N HCl, dried (magnesium sulfate), filtered, and concentrated to yield 0.115 g of the title product (48% yield). MS (ESI+) for C₂₄H₃₁F₂N₃O₂ m/z 432.18 [M+H]⁺.

### Step 9. ISOLATION OF N-((1S,2R)-1-(3,5-DIFLUOROBENZYL)-3-{[(4S)-6-ETHYL-1-METHYL-1,2,3,4-TETRAHYDROQUINOLIN-4-YL]AMINO}-2-HYDROXYPROPYL)ACETAMIDE AND N-((1S,2R)-1-(3,5-DIFLUOROBENZYL)-3-{[(4R)-6-ETHYL-1-METHYL-1,2,3,4-TETRAHYDROQUINOLIN-4-YL]AMINO}-2-HYDROXYPROPYL)ACETAMIDE

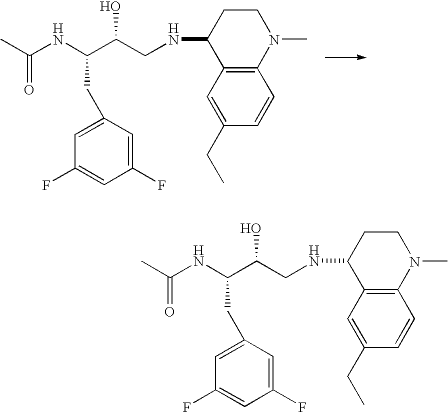

Silica gel chromatography of approximately 0.1 g of N-{(1S,2R)-1-(3,5-difluorobenzyl)-3-[(6-ethyl-1-methyl-1,2,3,4-tetrahydroquinolin-4-yl)amino]-2-hydroxypropyl}acetamide using 8:92 methanol/dichloromethane with 0.1% ammonium hydroxide eluent yielded 0.032 g of N-((1S,2R)-1-(3,5-difluorobenzyl)-3-{[(4S)-6-ethyl-1-methyl-1,2,3,4-tetrahydroquinolin-4-yl]amino}-2-hydroxypropyl)acetamide [R_{f} (MeOH/CH₂Cl₂/NH₄OH)=0.40; MS (ESI+) for C₂₄H₃₁F₂N₃O₂ m/z 432.2 [M+H]⁺]. Further purification of mixed fractions yielded 0.011 g of a 9:1 mixture of the 4R isomer (R_{f} (MeOH/CH₂Cl₂/NH₄OH)=0.35; MS (ESI+) for C₂₄H₃₁F₂N₃O₂ mm/z 432.2 [M+H]⁺] and the 4S isomer.

### EXAMPLE 4

### PREPARATION OF N-{1-(3,5-DIFLUORO-BENZYL)-3-[6-(2,2-DIMETHYL-PROPYL)-1-METHYL-1,2,3,4-TETRAHYDRO-QUINOLIN-4-YLAMINO]-2—HYDROXY-PROPYL}-ACETAMIDE

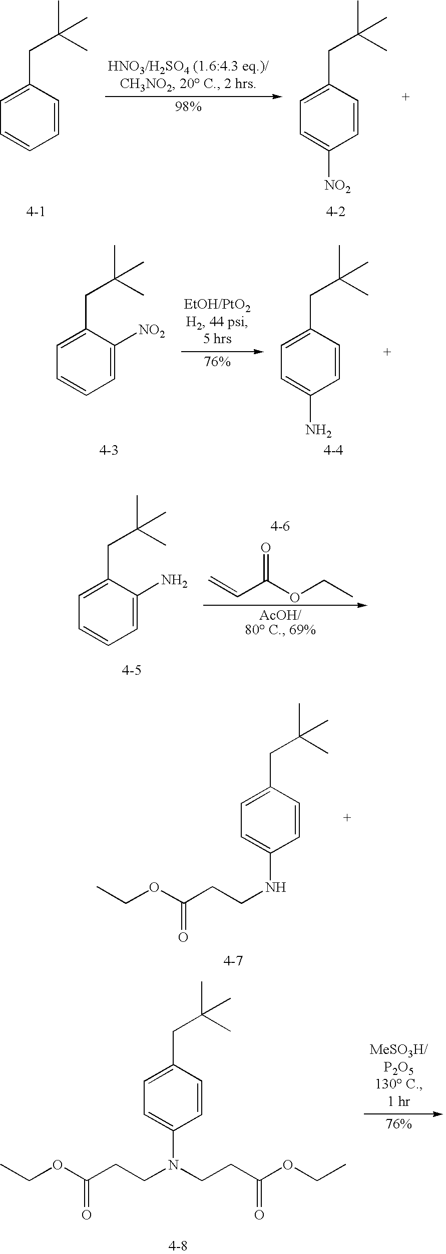

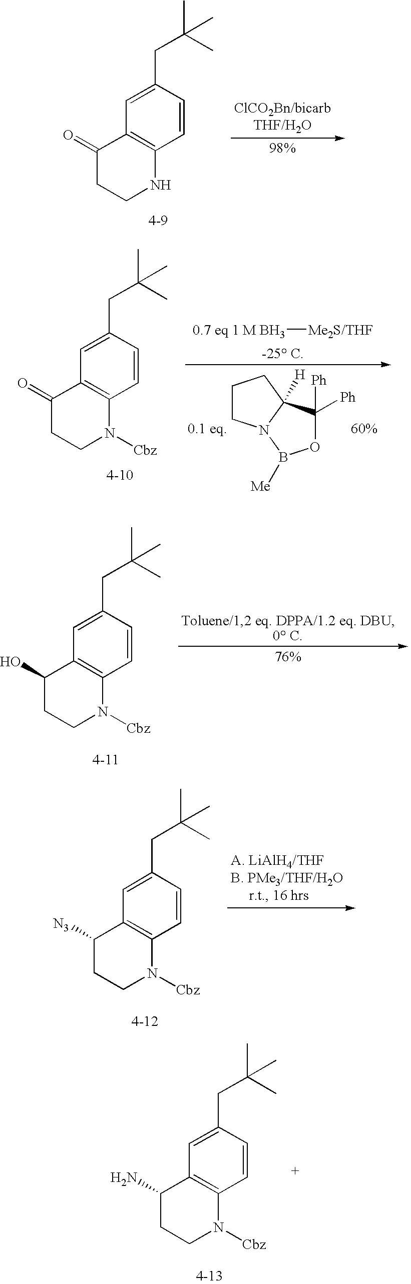

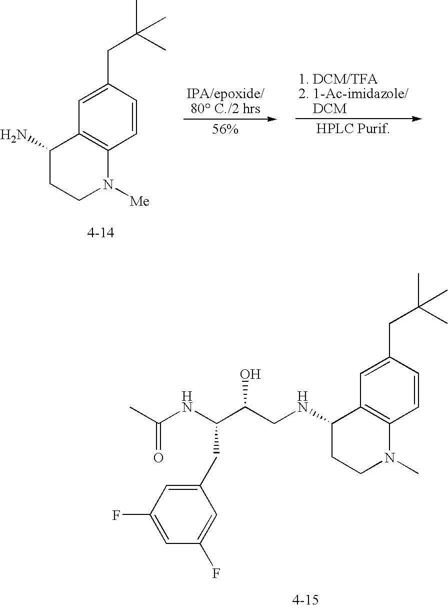

### Step 1. 1-Isobutyl-4-nitro-benzene and 1-(2,2-Dimethyl-propyl)-2-nitro-benzene

5.8 mL (92 mmol, 1.6 eq.) of conc. nitric acid at 0° C. was added dropwise over 10 min to 6.9 mL (249 mmol, 4.3 eq.) of conc. sulfuric acid. The mixture was then added to 8.6 g (57.9 mmol) of 2,2-di-methyl-propylbenzene in 45 mL of nitromethane, stirred at 0° C. for 2 h and overnight at room temperature.

The reaction was monitored by TLC, two new spots appeared at Rf=0.63 and 0.59. The mixture was poured into ice and extracted with dichloromethane. The combined extractants were then washed with bicarb, brine and water, dried with anhydrous sodium sulfate, and stripped of solvents yielding 11.02 g of 1-Isobutyl-4-nitro-benzene and 1-(2,2-Dimethyl-propyl)-2-nitro-benzene as an oil of o- and p-isomeric mixture (98%).

TLC (10% EtOAc/Hexane) Rf=0.63 and 0.59 while starting material at Rf=0.91. LCMS m/e=194.1(M+H), retention time=2.693 min (50% [B]:50% [A] to 95% [B]:5% [A] gradient in 3.33 min, then hold, at 1.5 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.)

### Step 2. 4-(2,2-Dimethyl-propyl)-phenylamine and 2-(2,2-Dimethyl-propyl)-phenylamine

240 mg (1.05 mmol, 4.2 mg/mmol) of platinum(IV)oxide was added to 11.0 g (57 mmol) of 1-Isobutyl-4-nitro-benzene and 1-(2,2-Dimethyl-propyl)-2-nitro-benzene in 20 mL of ethanol. The mixture was then saturated with hydrogen at 44 psi and shaken for 4 h. The mixture was then filtered through celite and the filtrates combined and stripped to give 9.26 g of the crude mixture, which was purified by flash column to give 3.47 g of a burgundy oil (o-isomer, 37%) and 3.92 g of 4-(2,2-Dimethyl-propyl)-phenylamine and 2-(2,2-Dimethyl-propyl)-phenylamine as a beige solid (p-isomer, 42%).

TLC (20% EtOAc/Hexane) Rf=0.65 and 0.48 while starting material at Rf=0.85 and 0.82. LCMS m/e=164.1 (M+H), retention time=1.937 min (20% [B]:80% [A] to 70% [B]:30% [A] gradient in 2.33 min, then hold, at 1.5 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.)

### Step 3. 3-[4-(2,2-Dimethyl-propyl)-phenylamino]-propionic acid ethyl ester and 3-[[4-(2,2-Dimethyl-propyl)-phenyl]-(2-ethoxycarbonyl-ethyl)-amino]-propionic acid ethyl ester

To 5.21 g of 4-(2,2-Dimethyl-propyl)-phenylamine and 2-(2,2-Dimethyl-propyl)-phenylamine (32 mmol) in 8 mL of acetic acid was added 3.2 g (32 mmol, 1 eq.) of ethyl acrylate and heated to 80° C. for 2 h, then 55° C. overnight.

The reaction was monitored by TLC and two new spots appeared at Rf=0.67 and 0.61. The mixture was partitioned by EtOAc/brine and dried over anhydrous sodium sulfate. Stripping the solvent gives 8.94 g of crude which was purified by flash column to give a 3.47 g mixture of 3-[4-(2,2-Dimethyl-propyl)-phenylamino]-propionic acid ethyl ester and 3-[[4-(2,2-Dimethyl-propyl)-phenyl]-(2-ethoxycarbonyl-ethyl)-amino]-propionic acid ethyl ester as a burgundy oil (69%).

TLC (20% EtOAc/Hexane) Rf=0.67 and 0.61 while starting material at Rf=0.47. LCMS m/e=264.2(M+H), retention time=2.639 min and LCMS m/e=364.2(M+H), retention time=3.524 min (20% [B]:80% [A] to 70% [B]:30% [A] gradient in 2.33 then hold, at 1.5 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.)

### Step 4. 6-(2,2-Dimethyl-propyl)-2,3-dihydro-1H-quinolin-4-one

To 4.9 g of phosphorus pentoxide (17.3 mmol, 1.3 eq.) was dissolved in 49 mL of methanesulfonic acid (756 mmol, 56 eq.) at 130° C. and the mixture allowed to cool to room temperature. 6.57 g of a mixture of 3-[4-(2,2-Dimethyl-propyl)-phenylamino]-propionic acid ethyl ester and 3-[[4-(2,2-Dimethyl-propyl)-phenyl]-(2-ethoxycarbonyl-ethyl)-amino]-propionic acid ethyl ester (13.5 mmol) was then added. The reaction heated to 130° C. for 1 h and monitored by TLC; a new spot appeared at Rf=0.61.

The mixture was poured into ice and treated with 1N NaOH to pH=10, then extracted with dichloromethane. The combined extractants were washed with brine and dried with anhydrous sodium sulfate, and stripped of solvents. The crude product was subjected to flash column purification to afford 4.97 g of 6-(2,2-Dimethyl-propyl)-2,3-dihydro-1H-quinolin-4-one as a tan oil, which was solidified upon standing (76%).

TLC (50% EtOAc/Hexane) Rf=0.61 while starting material at Rf=0.91 and 0.89. LCMS m/e=218.1(M+H), retention time=3.006 (20% [B]:80% [A] to 70% [B]:30% [A] gradient in 2.33 min, then hold, at 1.5 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.)

### Step 5. 6-(2,2-Dimethyl-propyl)-4-oxo-3,4-dihydro-2H-quinoline-1-carboxylic acid benzyl ester

To 5.6 g of sodium bicarbonate (66 mmol, 3 eq.) was dissolved in 10 mL water and added to 4.8 g of 6-(2,2-Dimethyl-propyl)-2,3-dihydro-1H-quinolin-4-one in 30 mL of THF; 4.12 g of benzyl chloroformate in 5 mL of THF was added slowly to the above mixture at 0° C. The mixture was stirred at room temperature for 2 h and the reaction was monitored by TLC; a new spot appeared at Rf=0.86.

The mixture was extracted with ether and washed with 5% citric acid and brine successively, dried with anhydrous sodium sulfate, stripping of solvent gives 7.69 g of the title compound 6-(2,2-Dimethyl-propyl)-4-oxo-3,4-dihydro-2H-quinoline-1-carboxylic acid benzyl ester as a tan oil (98%).

TLC (50% EtOAc/Hexane) Rf=0.86 (blue color under UV) while starting material at Rf=0.60. LCMS m/e=352.2(M+H), retention time=4.126 min (20% [B]:80% [A] to 70% [B]:30% [A] gradient in 2.33 min, then hold, at 1.5 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35°C.)

### Step 6. 6-(2,2-Dimethyl-propyl)-4-hydroxy-3,4-dihydro-2H-quinoline-1-carboxylic acid benzyl ester

2.1 mL of a mixture of 1M (S)-tetrahydro-1-methyl-3,3-diphenyl-1H, and 3H-pyrollo[1,2-c][1,3,2]oxazaborole/toluene (2.1 mmol, 0.1 eq.) was added to 7.5 g of 6-(2,2-Dimethyl-propyl)-4-oxo-3,4-dihydro-2H-quinoline-1-carboxylic acid benzyl ester (20.8 mmol) in 20 mL of THF. The reaction was cooled to −25° C. A solution of 1.4 mL of borane-methylsulfide (14.56 mmol, 0.7 eq.) in 25 mL of THF was added to the mixture dropwise over 20 min while the reaction was kept at −20° C. The mixture was then stirred at −20° C. for 1 h and monitored by TLC.

The reaction was quenched with 50 mL of methanol at −20° C. and allowed to warm to room temperature and stir overnight. The volatiles were removed in vacuo and the residue was purified by flash column to yield 4.4 g of the (R)— alcohol 6-(2,2-Dimethyl-propyl)-4-hydroxy-3,4-dihydro-2H-quinoline-1-carboxylic acid benzyl ester as a light tan oil (60%).

TLC (20% EtOAc/Hexarie) Rf=0.18 (blue color under UV long wave) while starting material at Rf=0.46. LCMS m/e=336.2(M−OH), retention time=3.692 (20% [B]: 80% [A] to 70% [B]: 30% [A] gradient in 2.33 min, then hold, at 1.5 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.)

### Step 7. 4-Azido-6-(2,2-dimethyl-propyl)-3,4-dihydro-2H-quinoline-1-carboxylic acid benzyl ester

3.2 mL of diphenylphosphorylazide (DPPA, 14.6 mmol, 1.2 eq.), followed by 2.2 mL of 1,8-diazabicyclo [5.4.0]undec-7-ere (DBU, 14.6 mmol, 1.2 eq.) in 20 mL of toluene, were added to 4.3 g of 6-(2,2-Dimethyl-propyl)-4-hydroxy-3,4-dihydro-2H-quinoline-1-carboxylic acid benzyl ester (12.2 mmol) in 25 mL of toluene at 0° C. The mixture was allowed to stir at 0° C. for 2 h and room temperature overnight and the reaction was monitored by TLC. The mixture was then filtered through a pad of sand-silica gel-sand contained in a Buchner funnel (eluted with 15% EtOAc/Hexane) to remove some precipitates and the volatiles were removed in vacuo to give 3.5 g of the crude S-azide 4-Azido-6-(2,2-dimethyl-propyl)-3,4-dihydro-2H-quinoline-1-carboxylic acid benzyl ester as a white solid (76%). This material was used directly in the next step without further purification.

TLC (20% EtOAc/Hexane) Rf=0.60 (blue color under UV long wave) while starting material at Rf=0.18. LCMS m/e=336.1(M−N₃), retention time=3.404 min (50% [B]: 50% [A] to 95% [B]: 5% [A] gradient in 3.33 min, then hold, at 1.5 mL/min, where [A]=0.1% trifluoroacetic acid in water;.[B]=0.1% trifluoroacetic acid in acetonitrile on a Phenomenex Luna C18 (2) 4.6 mm×30 cm column, 3 micron packing, 210 nm detection, at 35° C.)

### Step 8. 4-Amino-6-(2,2-dimethyl-propyl)-3,4-dihydro-2H-quinoline-1-carboxylic acid benzyl ester and 6-(2,2-Dimethyl-propyl)-1-methyl-1,2,3,4-tetrahydro-quinolin-4-ylamine

5.5 mL of 1 M trimethylphosphine/THF was added to 2.08 g of 4-Azido-6-(2,2-dimethyl-propyl)-3,4-dihydro-2H-quinoline-1-carboxylic acid benzyl ester (5.5 mmol) in 55 mL of THF and 0.1 mL of water at room temperature. The mixture was stirred overnight and monitored by TLC. The volatiles were removed in vacuo and the residue was purified by flash column to yield 2.39 g of 4-Amino-6-(2,2-dimethyl-propyl)-3,4-dihydro-2H-quinoline-1-carboxylic acid benzyl ester as a light tan oil (75%)

TLC (50% EtOAc/Hexane+20% MeOH/DCM, 1:1) Rf=0.35. LCMS m/e=336.1(M−NH₂), retention time=2.472 and 0.28 g of 6-(2,2-Dimethyl-propyl)-1-methyl-1,2,3,4-tetrahydro-quinolin-4-ylamine, a N-methyl tetraquinolin amine, was also isolated as a tan oil. LCMS m/e=216.1(M−NH₂), retention time=0.333 (20% [B]: 80% [A] to 70% [B]: 30% [A] gradient in 2.33 min, then hold, at 1.5 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.)

### Step 9. N-{1-(3,5-Difluoro-benzyl)-3-[6-(2,2-dimethyl-propyl)-1-methyl-1,2,3,4-tetrahydro-quinolin-4-ylamino]-2-hydroxy-propyl}-acetamide

N-{1-(3,5-Difluoro-benzyl)-3-[6-(2,2-dimethyl-propyl)-1-methyl-1,2,3,4-tetrahydro-quinolin-4-ylamino]-2-hydroxy-propyl}-acetamide was prepared according to the steps described above.

LCMS m/e=496.2(M+Na), retention time=2.039 (20% [B]: 80% [A] to 70% (B]: 30% [A] gradient in 2.33 min, then hold, at 1.5 mL/min, where [A]=0.1% trifluoroacetic acid in water; [B]=0.1% trifluoroacetic acid in acetonitnle on a Phenomenex-Luna C18 (2) 4.6 mm×30 cm column, 3 micron packing, 210 nm detection, at 35° C.)

¹H NMR (CDCl₃) δ 7.56 (s, 1 H0, 7.02-6.99 (d, J=8.8 Hz, 1H), 6.89 (m, 1 H), 6.74 (m, 1H), 6.68-6.66 (m, 1H), 6.62-6.59 (m, 1H), 4.63 (s, 1H), 4.39-4.32 (m, 1H), 4.12-4.07 (m, 1H), 3.94 (m, 1H), 3.40-3.35 (m, 1H), 3.18-3.15 (m, 1H), 3.05-2.98 (m, 1H), 2.87 (s, 3H), 2.81-2.62 (m, 1 H), 2.45-2.37 (m, 1 H), 2.33 (s, 2H),2.32-2.28 (m, 1H), 1.85 (s, 3H), 0.98 (s, 2H), 0.92 (s, 2H), 0.84 (s, 9H).

¹³C NMR (CDCl₃) δ 164.1, 158.9, 133.4, 124.8, 120.8, 111.8, 100.1, 86.7, 83.6, 77.4, 77.0, 76.6, 52.8, 38.3, 31.6, 29.

### EXAMPLE 5

### PREPARATION OF N-{(1S,2R)-1-(3,5-DIFLUOROBENZYL)-2-HYDROXY-3-[(6-NEOPENTYL-1,2,3,4-TETRAHYDROQUINOLIN-4-YL)AMINO]PROPYL}ACETAMIDE

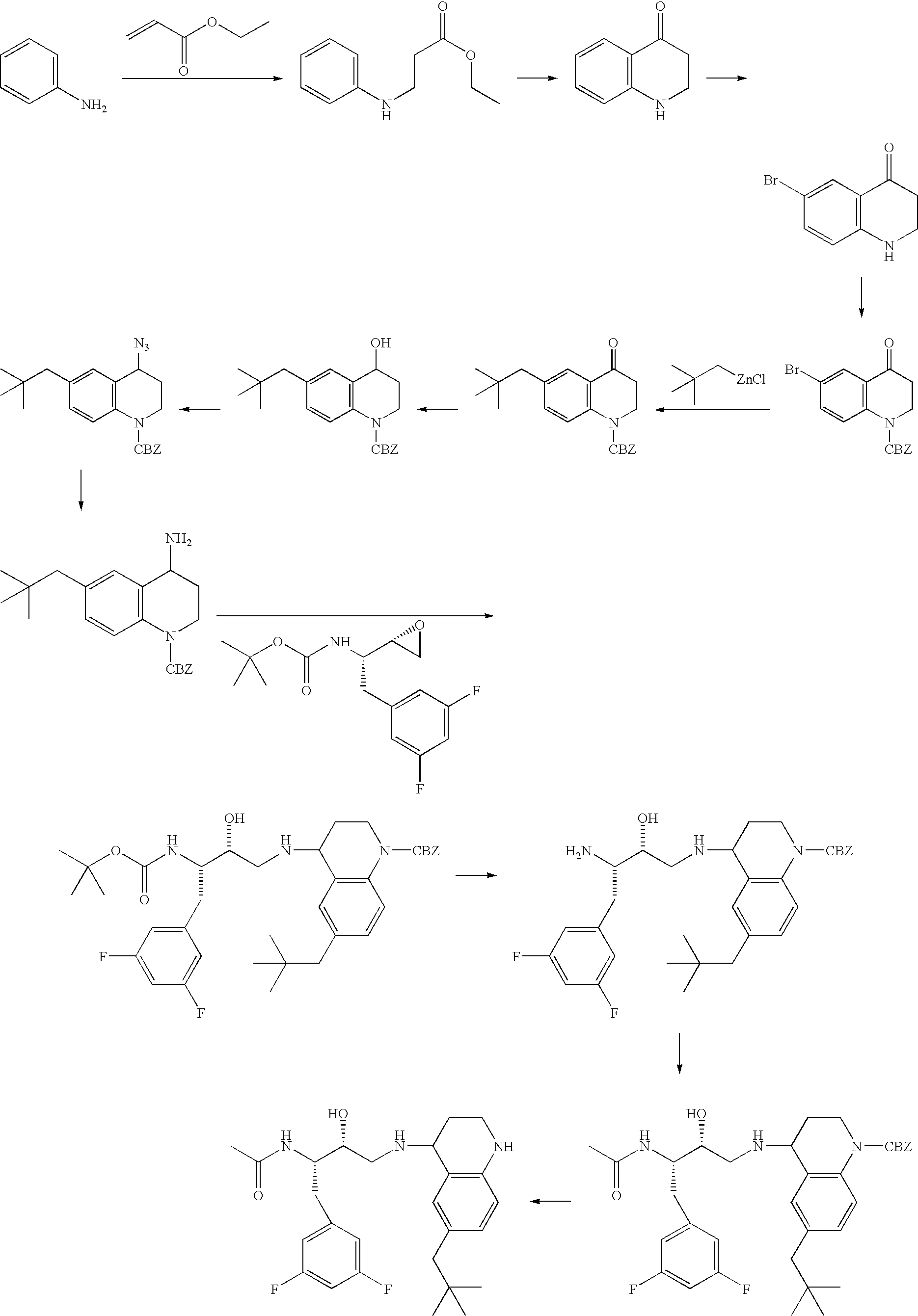

### Step 1. PREPARATION OF ETHYL N-PHENYL-BETA-ALANINATE

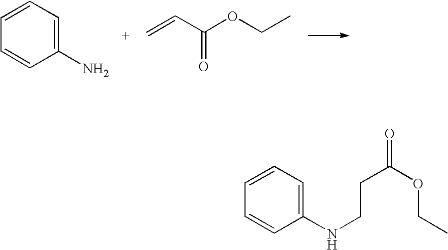

Ethyl N-phenyl-beta-alaninate was prepared essentially according to the method of Example 2, step 1. The crude product was purified by chromatography on silica gel (eluent: 15% ethyl acetate in heptane with 0.25% TFA). The purified mixture comprised the mono and di-ester products (1:1) which were used in the next step without further purification. MS (ESI+) for C₁₁H₁₅NO₂ m/z 193.99 [M+H]⁺.

### Step 2. PREPARATION OF 2,3-DIHYDROQUINOLIN-4(1H)-ONE

2,3-Dihydroquinolin-4(1 H)-one was prepared essentially according to the method of Example 2, step 2. The crude product was purified by column chromatography using a 20-30% ethyl acetate in heptane gradient. MS (ESI+) for C₉H₉NO m/z 147.96 [M+H]⁺.

### Step 3. PREPARATION OF 6-BROMO-2,3-DIHYDROQUINOLIN-4(1H)-ONE

N-Bromosuccinimide (3.63 g) was added to a solution of 2,3-dihydroquinolin-4(1 H)-one (2.94 g) in dichloromethane (25 mL). The mixture was stirred at room temperature for 1.5 h, and partitioned between aqueous sodium bicarbonate and dichloromethane. The organic layer was washed with brine, dried (sodium sulfate), filtered, and concentrated. Silica gel chromatography of the concentrate (eluent 35% ethyl acetate in heptane) yielded 4.14 g of the title compound. MS (ESI−) for C₉H₈BrNO m/z 225.77 [M−H]⁻.

### Step 4. PREPARATION OF BENZYL 6-BROMO-4-OXO-3,4-DIHYDROQUINOLINE-1 (2H)-CARBOXYLATE

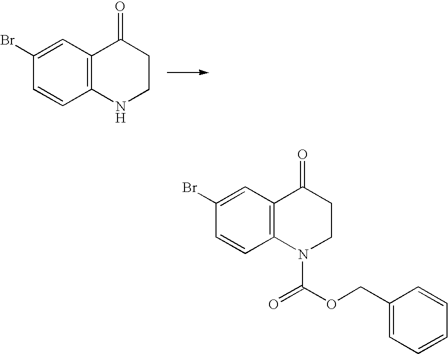

Benzyl 6-bromo-4-oxo-3,4-dihydroquinoline-1 (2H)-carboxylate was prepared essentially according to the method of Example 2, step 3. ¹H NMR (CDCl₃) δ 2.78 (t, J=7 Hz, 2 H), 4.22 (t, J=6 Hz, 2H), 5.28 (s, 2 H), 7.40 (m, 5H), 7.58 (dd, J=2, 9 Hz, 1 H), 7.75 (d, J=9 Hz, 1 H), 8.10 (d, J=2 Hz, 1 H).

### Step 5. PREPARATION OF BENZYL 6-NEOPENTYL-4-OXO-3,4-DIHYDROQUINOLINE-1 (2H)-CARBOXYLATE

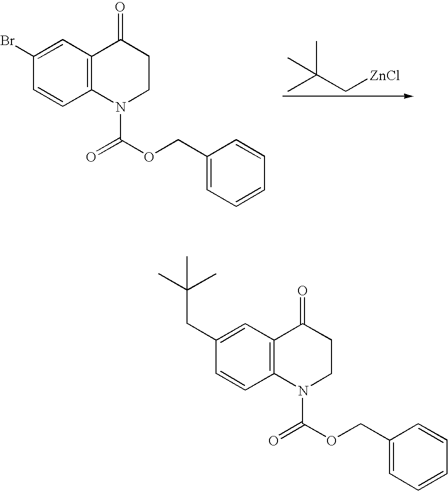

Benzyl 6-bromo-4-oxo-3,4-dihydroquinoline-1(2H)-carboxylate (3.10 g) and dichloro[1,1′-bis(diphenylphosphino)ferrocene]palladium(ii)dichloromethane adduct (0.35 g) were combined in a round bottom flask. The mixture was put under high vacuum and purged with N₂(g). A 0.5. M solution of bromo(neopentyl)zinc (55 mL), prepared using the procedure of Negishi et al. Tet Lett., 1983, 24, 3823-3824, was added to the mixture. The reaction was stirred at room temperature for 2 days. The reaction had not gone to completion, so an additional 10 mL of bromo(neopentyl)zinc solution was added and the mixture was stirred for one additional day. The mixture was then partitioned between ethyl acetate and aqueous ammonium chloride, dried (magnesium sulfate), filtered, and concentrated. Silica gel chromatography (eluent: 20% ethyl acetate in heptane) yielded 2.17 g of the title compound. MS (ESI+) for C₂₂H₂₅NO₃ m/z 353.17 [M+H]⁺.

### Step 6. PREPARATION OF BENZYL 4-HYDROXY-6-NEOPENTYL-3,4- DIHYDROQUINOLINE-1 (2H)-CARBOXYLATE

Benzyl 4-hydroxy-6-neopentyl-3,4-dihydroquinoline-1(2H)-carboxylate was prepared essentially according to the method of preparing 3,4-dihydro-2H-chroman-4-ylamine described in Example 50, step 2 below: MsCl (2.1 mL, 27 mmol) was added to a CH₂Cl₂ (80 mL) solution of chroman-4-ol (3.1 g, 20.6 mmol) and DIEA (8 mL, 42 mmol), at 0° C., via syringe. After addition was complete, the cold bath was removed and stirring continued at room temperature. After 15 h, the CH₂Cl₂ was removed in vacuo and the residue was dissolved in 80 mL of DMF. NaN₃ (1.8 g, 27 mmol) was then added and the mixture was heated to 75° C. (oil bath) for 5 h, then cooled to room temperature. The mixture was diluted with Et₂O (400 mL), washed with 1 N HCl, NaHCO₃, and brine, then dried (sodium sulfate), filtered, and concentrated in vacuo to yield the azide as a yellow oil. ¹H NMR (400 MHz, CDCl₃) δ 7.27-7.21 (m, 2 H), 6.97-6.87 (m, 2 H), 4.61 (appt, J=3.84 Hz, 1 H), 4.31-4.19 (m, 2 H), 2.18 (m, 1 H), 2.03 (m, 1 H). MS (ESI−) for C₉H₁₀N₃) m/z 173.0 [M−H]⁻. The crude azide was dissolved in 60 mL of THF and PPh₃ (6.5 g, 25 mmol) was added. The mixture was then stirred at room temperature for 30 min. The mixture was treated with 8 mL of H₂O and heated to 60° C. (oil bath) overnight. The mixture was concentrated in vacuo and the resulting residue treated with 1 N HCl. The aqueous mixture was extracted with CH₂Cl₂, adjusted to pH 12 with NaOH, and re-extracted with CH₂Cl₂. The second CH₂Cl₂ layers were combined; dried (sodium sulfate), filtered, and concentrated in vacuo to yield 3,4-dihydro-2H-chroman-4-ylamine as a slightly yellow oil. HRMS (ESI+) calc'd for C₉H₁₁NO m/z 150.0919 [M+H]⁺; found 150.0920.

The crude product was purified ¹H NMR (CDCl₃) δ 0.90 (s, 9 H), 1.80 (s, 1 H), 2.06 (m., 2 H), 2.45 (s, 2H), 3.68 (m, 1 H), 4.12 (m, 1 H), 4.75 (t, J=4 Hz, 1 H), 5.24 (dd, J=12, 17 Hz, 2 H), 7.02 (dd, J=2, 9 Hz, 1 H), 7.12 (d, J=2 Hz, 1 H), 7.35 (m, 5 H), 7.76 (d, J=8 Hz, 1 H).

### Step 7. PREPARATION OF BENZYL 4-AMINO-6-NEOPENTYL-3,4-DIHYDROQUINOLINE-1 (2H)-CARBOXYLATE

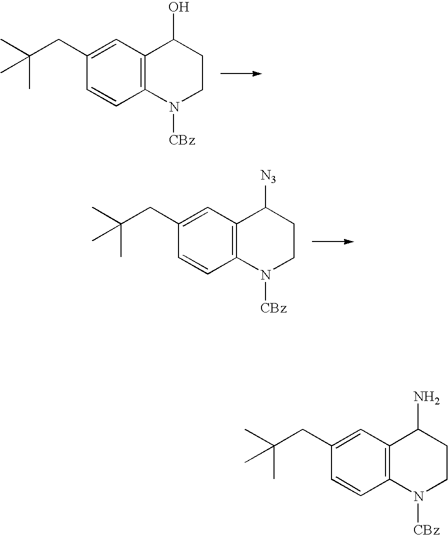

Benzyl 4-amino-6-neopentyl-3,4-dihydroquinoline-1(2H)-carboxylate was prepared essentially according to the method of step 6. First, the azide was prepared and chromatographed on silica gel (eluent: 15% ethyl acetate in heptane). ¹H NMR (CDCl₃) δ 0.091 (s, 9 H), 2.09 (m, 2 H), 2.46 (s, 2 H), 3.66 (m, 1 H), 4.14 (m, 1 H), 4.58 (t, J=4 Hz, 1 H), 4.24 (dd, J=12, 15 Hz, 2 H), 7.03 (d, J=2 Hz, 1 H), 7.06 (dd, J=2, 9 Hz, 1 H), 7.35 (m, 5 H), 7.86 (d, J=8 Hz, 1 H).

Second, the azide was reduced using PMe₃. The resulting amine was purified by silica gel chromatography (eluent: 2.5% methanol in dichloromethane with 0.5% ammonium hydroxide). MS (ESI+) for C₂₂H₂₈N₂O₂ m/z 353.19 [M+H]⁺.

### Step 8. PREPARATION OF BENZYL 4-{[(2R,3S)-3-AMINO-4-(3,5-DIFLUOROPHENYL)-2-HYDROZYBUTYL]AMINO)-6-NEOPENTYL-3,4-DIHYDROQUINOLINE-1 (2H)-CARBOXYLATE

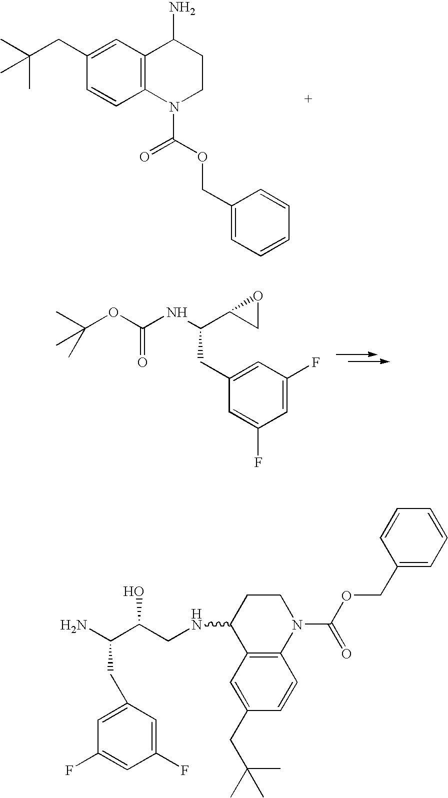

tert-Butyl (1S)-2-(3,5-difluorophenyl)-1-[(2S)-oxiran-2-yl]ethylcarbamate (0.75 g) was added to a solution of benzyl 4-amino-6-neopentyl-3,4-dihydroquinoline-1(2H)-carboxylate (1.31 g) in isopropanol (25 mL) and the mixture was heated at 90° C. for 45 min. The temperature was reduced to 60° C. and the mixture was allowed to stir overnight. An additional 0.36 g of tert-butyl (1S)-2-(3,5-difluorophenyl)-1-[(2S)-oxiran-2-yl]ethylcarbamate was added to the mixture. The mixture was then heated to 80° C. for 5 h. The mixture was cooled to room temperature and the solvent was removed under reduced pressure. The residue was partitioned between water and ethyl acetate. The organic layers were dried (magnesium sulfate), filtered, and concentrated. A silica gel column was run to attempt to separate the diastereomers using a gradient of 2-4% MeOH in dichloromethane with 0.25% NH₄OH. The first fraction contained a 70:30 mixture of the two diastereomers; the second fraction was a 50:50 mix of the diastereomers. The Boc groups were removed by dissolving each fraction in a minimal amount of dichloromethane and adding 15 mL of 2N HCl in ether to each of the two mixtures. The mixtures were stirred for 2 h and concentrated under reduced pressure. The mixtures were then partitioned between 1N sodium hydroxide and ethyl acetate, dried (magnesium sulfate), filtered, and concentrated to give 0.23 g of the 70:30 title compound mixture and 0.30 g of the 50:50 mixture. MS (ESI+) for C₃₂H₃₉F₂N₃O₃ m/z 552.32 [M+H]⁺ for the 70:30 mixture and m/z 552.27 [M+H]⁺ for the 50:50 mixture. Each of these mixtures was carried on separately to final product; the following procedures illustrate that for the 70:30 mixture only.

### Step 9. PREPARATION OF BENZYL 4-{[(2R,3S)-3-(ACETYLAMINO)-4(3,5-DIFLUOROPHENYL)-2-HYDROZYBUTYL]AMINO}-6-NEOPENTYL-3,4-DIHYDROQUINOLINE-1(2H)-CARBOXYLATE

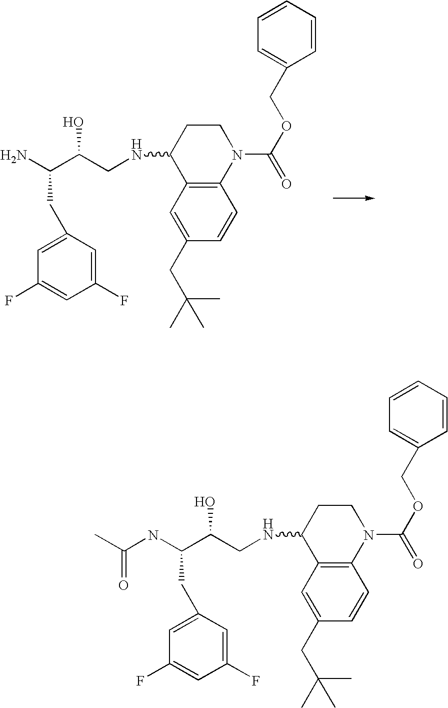

N,N-Diacetyl-O-methylhydroxylamine (0.064 g) was added to a solution of benzyl 4-{[(2R,3S)-3-amino-4-(3,5-difluorophenyl)-2-hydroxybutyl]amino}-6-neopentyl-3,4-dihydroquinoline-1(2H)-carboxylate (0.226 g) in dichloromethane (5 mL). The mixture was stirred for 72 h at room temperature. The solvent was removed under reduced pressure and the residue was partitioned between 1N HCl and ethyl acetate, dried (magnesium sulfate), filtered, and concentrated to give 0.243 g of the title compound (99% yield). MS (ESI+) for C₃₄H₄₁F₂N₃O₄ m/z 594.31 [M+H]⁺.

### Step 10. PREPARATION OF N-{(1S,2R)-1-(3,5-DIFLUOROBENZYL)-2-HYDROXY-3-[(6-NEOPENTYL-1,2,3,4-TETRAHYDROQUINOLIN-4-YL)AMINO]PROPYL}ACETAMIDE

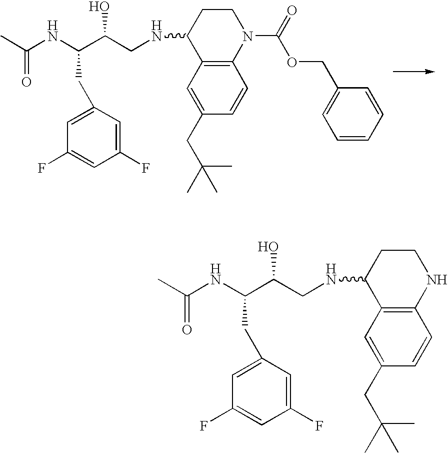

1N HCl (1.0 mL) and 10% palladium on carbon (0.030 g) were added to a solution of benzyl 4-{[(2R,3S)-3-(acetylamino)-4-(3,5-difluorophenyl)-2-hydroxybutyl]amino}-6-neopentyl-3,4-dihydroquinoline-1(2H)-carboxylate (0.242 g) in EtOH (30 mL). The mixture was degassed with N₂ for 5 min. The mixture was hydrogenated under 47 psi of H₂ and shaken for 4.5 h. The palladium was filtered off using Celite® and the solution was concentrated under reduced pressure. The residue was then partitioned between water and ethyl acetate. The organic layers were washed with aqueous sodium bicarbonate, dried with magnesium sulfate, filtered, and concentrated. A silica gel column (eluent: 4% MeOH in dichloromethane with 0.25% NH₄OH) yielded 0.095 g of the title compound. MS (ESI+) for C₂₆H₃₅F₂N₃O₂ m/z 460.27 [M+H]⁺.

### EXAMPLE 6

### PREPARATION OF N-[1-(3,5-DIFLUOROBENZYL)-3-(3-ETHYL-5,6,7,8-TETRAHYDROQUINOLIN-5-YLAMINO)-2-HYDROXYPROPYL]-ACETAMIDE

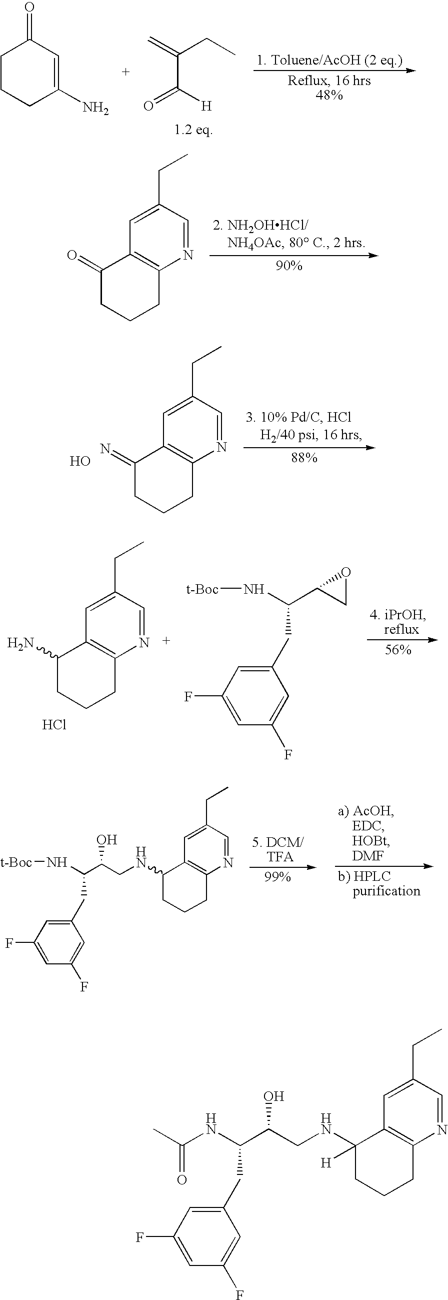

See Albright, J. D., J. Heterocycl. Chem., 2000, 37, 41-6, for a general reference on preparing pyridyl tetralin compounds.

### Step 1

Acetic acid (6 mL) and toluene (25 mL) were added to 3-amino-2-cyclohexan-1-one (5.5 g, 49.5 mmol) and 2-ethyl acrolein (5 g, 59.4 mmol, 1.2 eq.). The reaction mixture was heated to reflux overnight. The reaction was monitored by TLC to show formation of a new spot with Rf=0.73 (50% MeOH/DCM+20% EtOH/Hexane.). Solvent was removed and the residue taken up in toluene, which was removed again. The residue was extracted with DCM (2×), washed with saturated NaHCO₃, dried (sodium sulfate), and concentrated to give 9.38 g crude dark tan oil. This crude oil was extracted with hot hexanes. The extracts were concentrated and dried in vacuo to give a light tan solid. (4.13 g, 23.6 mmol, 48%). MS (CI) m/z 176.1 [M+H]⁺.

### Step 2

The oxime was formed using procedures described elsewhere in the application, including, for example, Example 3, step 4. yield: 90%; MS (CI) m/z 191.1 [M+H]⁺.

### Step 3

Reduction of the oxime was performed essentially according to procedures described elsewhere in the application, including, for example, Example 3, step 5. yield: 88%; MS (CI) m/z 177.1 [M+H]⁺.

### Step 4

The amine hydrochloride salt was converted to the free base by partitioning between 1 N NaOH and EtOAc. The free base solution was then concentrated and used in the epoxide opening reaction as previously described in, for example, Example 3: yield: 56%; MS (CI) m/z 476.2 [M+H]⁺.

### Step 5

Boc deprotection and acetylation was performed as previously described in, for example, Example 3. Reverse phase HPLC was effective in the resolution of the two diastereomers:

N-(1S, 2R)-[1-(3,5-Difluorobenzyl)-3-((5S)-3-ethyl-5,6,7,8-tetrahydroquinolin-5-ylamino)-2-hydroxypropyl]-acetamide: MS (CI) m/z 418.2 [M+H]⁺.

### EXAMPLE 7

### PREPARATION OF PHENACYL-2-HYDROXY-3-DIAMINOALKANES AND BENZAMIDE-2-HYDROXY-3-DIAMINOALKANES

An example of one of many various processes that can be used to prepare the compounds of the invention is set forth in the following scheme.

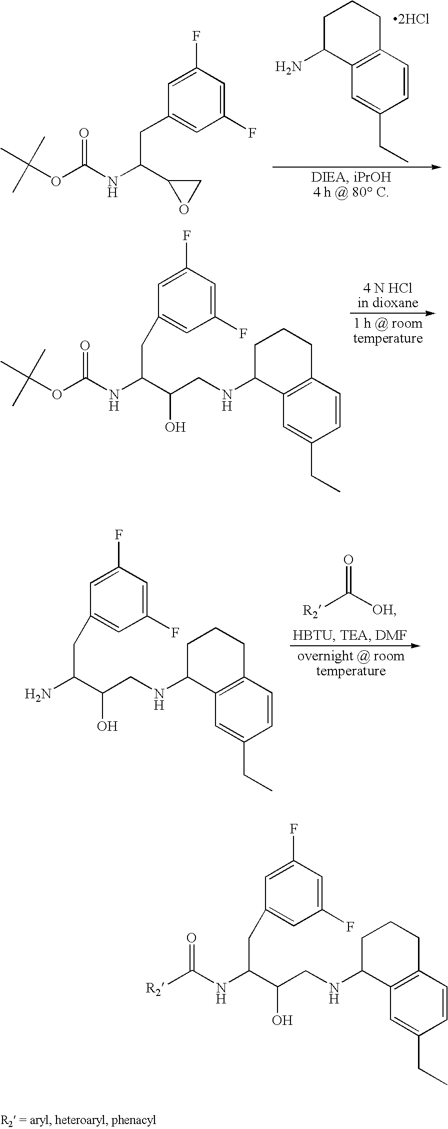

### Step 1

Opening of the epoxide was carried out with a 1:1 molar ratio of the erythro epoxide to the bicyclic C-terminal piece in a 20 mL reaction vial. Diisopropylethylamine (4 eq) were added to the vial followed by isopropanol (10 mL). The reaction was heated to 80° C. The isopropanol and diisopropylethylamine were evaporated under N₂(g).

### Step 2

The Boc-group deprotection in the second step was accomplished by using 3 equivalents of 4 N HCl in dioxane with respect to the amount of starting material. This reaction was run at room temperature for 1 hour. The dioxane was then evaporated under N₂(g).

### Step 3

The starting amine (0.07 mmol) was placed in each reaction vial. Then triethylamine (0.14 mmol, 2 eq) and the carboxylic acid (0.077 mmol, 1.1 eq) were added. The starting reagents were then dissolved in DMF (1.5 mL). Finally, HBTU (0.077 mmol, 1.1 eq) in DMF was added. Each reaction was run overnight at room temperature. LC/MS analysis for each reaction was performed via an Agilent 1100 HPLC, utilizing a Thermo-Hypersil C18 50×3 mm 5 micron column, coupled to a. Thermo-Finnigan LCQ MS. Final purification of each product was performed via a Varian Pro Star Preparative HPLC utilizing a Phenomenex C18 60×21.2 mm 5 micron column.

### EXAMPLE 8

### PREPARATION OF N-((1S,2R)-1-(3,5-DIFLUOROBENZYL)-2-HYDROXY-3-{[(1S)-2-(HYDROXYMETHYL)-7-NEOPENTYL-1,2,3,4-TETRAHYDRONAPHTHALEN-1-YL]AMINO}PROPYL)ACETAMIDE

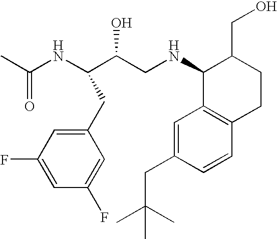

### Step 1: PREPARATION OF 7-(2,2-DIMETHYL-PROPYL)-1-HYDROXY-3,4-DIHYDRO-NAPHTHALENE-2-CARBOXYLIC ACID METHYL ESTER.

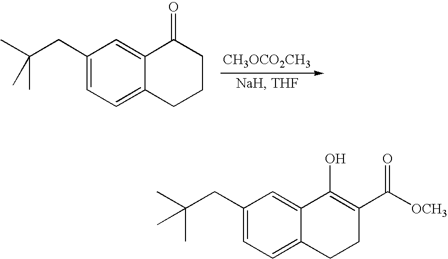

Sodium hydride (60%, 1.49 g, 37.1 mmol) followed by dimethyl carbonate (2.73 g, 30 mmol) was added to a solution of tetralone (2.16 g, 10 mmol) in tetrahydrofuran (50 mL). The reaction mixture was heated at reflux for 3 h and then allowed to cool to room temperature and quenched with acetic acid (3.6 mL). The solvent was removed under reduced pressure and the residue was diluted with ethyl ether (100 mL) and water (50 mL). The organic layer was separated and the aqueous layer was extracted with ethyl ether. The combined extracts were washed with saturated sodium chloride, dried (sodium sulfate), filtered, and concentrated under reduced pressure. Flash column chromatography (silica gel, 10-20% ethyl acetate/hexanes) provided the desired product (2.50 g, 91%): ¹H NMR (300 MHz, CDCl₃) δ 12.48 (s, 1H), 7.60 (s, 1H), 7.17-7.08 (m, 2H), 3.85 (s, 3H), 2.84-2.79 (m, 2H), 2.62-2.57 (m, 2H), 2.54 (s, 2H), 0.94 (s, 9H).

### Step 2: PREPARATION OF 2-(TERT-BUTYL-DIMETHYL-SILANYLOXYMETHYL)-7-(2,2-DIMETHYL-PROPYL)-3,4-DIHYDRO-2H-NAPHTHALEN-1-ONE

Lithium aluminum hydride (1 M in tetrahydrofuran, 9 mL, 9 mmol) was added to an ice-cooled solution of 7-(2,2-dimethyl-propyl)-1-hydroxy-3,4-dihydro-naphthalene-2-carboxylic acid methyl ester (2.49 g, 9.07 mmol) in tetrahydrofuran (20 mL). The reaction mixture was stirred at 0° C. for 2 h and then quenched with saturated ammonium chloride and ethyl acetate. The resulting emulsion was filtered through diatomaceous earth. The organic layer was separated and the aqueous layer was extracted with ethyl acetate. The combined extracts were washed with saturated sodium chloride, dried (sodium sulfate), filtered, and concentrated under reduced pressure. Flash column chromatography (silica gel, 10-20% ethyl acetate/hexanes) provided hydroxymethyl tetralone (1.55 g, 70%): ¹H NMR (300 MHz, CDCl₃) δ 7.78 (d, J=1.4 Hz, 1H), 7.27 (dd, J=7.8, 1.4 Hz, 1H), 7.16 (d, J=7.8 Hz, 1H), 4.00-3.90 (m, 1H), 3.85-3.75 (m, 1H), 3.20-3.10 (m, 1H), 3.08-2.90 (m, 2H), 2.75-2.60 (m, 1H), 2.52 (s, 2H), 2.15-2.05 (m, 1H), 2.00-1.85 (m, 1H), 0.90 (s, 9H).

Imidazole (500 mg, 7.25 mmol) followed by tert-butyidimethylsilyl chloride (1.03 g, 6.64 mmol) was added to a solution of hydroxymethyl tetralone (1.50 g, 6.09 mmol) in N,N-dimethyl formamide (6 mL). The reaction mixture was stirred at room temperature for 2 h and then diluted with 1:1 hexanes/ethyl acetate (100 mL). The mixture was washed successively with 1N hydrochloric acid, water, saturated sodium bicarbonate, and saturated sodium chloride, and dried (sodium sulfate), filtered, and concentrated under reduced pressure to provide 2-(tert-Butyl-dimethyl-silanyloxymethyl)-7-(2,2-dimethyl-propyl)-3,4-dihydro-2H-naphthalen-1-one (2.20 g, 99% crude yield): ¹H NMR (300 MHz, CDCl₃) δ 7.76 (d, J=1.8 Hz, 1H), 7.23 (dd, J=7.8, 1.8 Hz, 1H), 7.14 (d, J=7.8 Hz, 1H), 4.16-4.08 (m, 2H), 3.90-3.84 (m, 1H), 3.01-2.95 (m, 2H), 2.68-2.60 (m, 1H), 2.51 (s, 2H), 2.42-2.33 (m, 1H), 2.03-1.95 (m, 1H), 0.89 (s, 9H), 0.87 (s, 9H), 0.07 (s, 3H), 0.06 (s, 3H). This material was used in the next step without further purification.

### Step 3: PREPARATION OF 2-(TERT-BUTYL-DIMETHYL-SILANYLOXYMETHYL)-7-(2,2-DIMETHYL-PROPYL)-1,2,3,4-TETRAHYDRO-NAPHTHALEN-1-OL

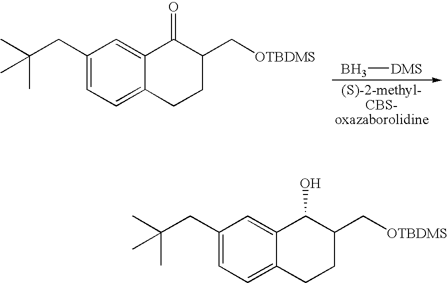

(S)-2-methyl-Cbs-oxazaborolidine (1 M in toluene, 0.61 mL, 0.61 mmol) and a solution of borane-methyl sulfide complex (2 M in tetrahydrofuran, 2.15 mL, 4.3 mmol) in tetrahydrofuran (5 mL) were added to a −30° C. cooled solution of 2-(tert-butyl-dimethyl-silanyloxymethyl)-7-(2,2-dimethyl-propyl)-3,4-dihydro-2H-naphthalen-1-one (2.20 g, 6.09 mmol) in tetrahydrofuran (20 mL). The reaction temperature was kept between −20 to −5° C. for 5 h. The reaction mixture was quenched with methanol (8.3 mL) at −5° C., allowed to warm to room temperature, and stirred overnight. The solvent was removed under reduced pressure. Flash column chromatography (silica gel, 0-5% ethyl acetate/hexanes) recovered 790 mg of ketone and provided chiral 2-(tert-butyl-dimethyl-silanyloxymethyl)-7-(2,2-dimethyl-propyl)-1,2,3,4-tetrahydro-naphthalen-1-ol (980 mg, 70%): ¹H NMR (300 MHz, CDCl₃) δ 7.14 (s, 1H), 7.03-6.96 (m, 2H), 4.84 (d, J=2.5 Hz, 1H), 3.92-3.82 (m, 2H), 3.04 (d, J=3.7 Hz, 1H), 2.92-2.67 (m, 2H), 2.46 (s, 2H), 2.04-1.86 (m, 2H), 1.75-1.63 (m, 1H), 0.91 (s, 9H), 0.90 (s, 9H), 0.10 (s, 3H), 0.09 (s, 3H).

### Step 4: [1-AMINO-7-(2,2-DIMETHYL-PROPYL)-1,2,3,4-TETRAHYDRO-NAPHTHALEN-2-YL]-METHANOL

The alcohol was converted into an amine essentially according to the method of preparing (6-bromo-isochromen-4-yl)-carbamic acid tert-butyl ester as described in Example 77, step 4 below: Diphenyphosphoryl azide (2.11 mL, 9.8 mmol) was added at 0° C. to a solution of 6-bromo-isochromen-4-ol (1.87 g, 8.16 mmol) in toluene (17 mL). A mixture of 1,8-diazabicyclo[5.4.0]undec-7-ene (1.46 mL, 9.8 mmol) in toluene (5.0 mL) was added to the reaction drop-wise over 0.5 h. The reaction mixture was then stirred at room temperature overnight then passed through a plug of silica and the plug rinsed with 6:1 hexanes/ethyl acetate. The combined filtrates were concentrated under reduced pressure to provide the azide as a yellow oil: ¹H NMR (300 MHz, CDCl₃) δ 7.46-7.33 (m, 3H), 4.76 (ABq, J=15.5 Hz, 2H), 4.22-4.16 (m, 3H), 3.93 (dd, J=11.7, 2.6 Hz, 1H). A solution of lithium aluminum hydride (391 mg, 9.79 mmol) in a minimum amount of tetrahydrofuran (2.0 mL) was added dropwise at 0° C. to a solution of the azide in tetrahydrofuran (30 mL) and the reaction mixture was heated at reflux for 1 h. The reaction mixture was cooled to room temperature and quenched with water (0.5 mL), 15% sodium hydroxide (1.2 mL), water (0.5 mL) and the reaction mixture stirred at room temperature for 1 h. The resulting mixture was then passed through a plug of silica and the plug rinsed with ether. The combined filtrates were concentrated under reduced pressure to yield an oil which was dissolved in a minimum amount of ethyl acetate. Hydrogen chloride (3.0 mL, 4 N in 1,4-dioxane, 12 mmol) was added and the reaction was stirred at room temperature overnight. The reaction mixture was vacuum filtered to yield the desired amine salt (1.54 g, 72% for 2 steps) as a white solid: ¹H NMR (300 MHz, CDCl₃) δ 7.54-7.44 (m, 2H), 7.37 (s, 1 H), 4.80 (ABq, J=15.5 Hz, 2H), 4.42 (d, J=12.8 Hz, 1H), 4.34 (s, 1H), 3.87 (dd, J=12.8, 2.2 Hz, 1H), 3.66 (s, 3 H); ESI MS m/z 228 [C₉H₁₀BrNO+H]⁺. Di-tert-butyl dicarbonate (1.40 g, 6.40 mmol) was added in portions to a solution of the amine (1.54 g, 5.82 mmol) in acetonitrile (25 mL) and N,N-diisopropylethylamine (4.0 mL, 23.28 mmol). The reaction mixture stirred at room temperature overnight, concentrated under reduced pressure, then partitioned between ethyl acetate and water. The organic phase was dried (sodium sulfate), filtered, and concentrated under reduced pressure to yield a yellow syrup. Purification by flash column chromatography over silica (80:20 hexanes/ethyl acetate) yielded the desired product (1.05 g, 55%) as a white solid.

However, the resulting amine was not protected in this step. First the alcohol was converted to the azide, which was purified by flash column chromatography (silica gel, 0-5% ethyl acetate/hexanes). ¹H NMR (300 MHz, CDCl₃) δ 7.15 (s, 1H), 7.03-6.97 (m, 2H), 4.42 (d, J=2.5 Hz, 1H), 3.75 (dd, J=10.1, 5.1 Hz, 1H), 3.67 (dd, J=10.1, 4.8 Hz, 1H), 2.81-2.67 (m, 2H), 2.48 (s, 2H), 2.07-1.98 (m, 2H), 1.80-1.67 (m, 1H), 0.91 (s, 9H), 0.89 (s, 9H), 0.08 (s, 3H), 0.07 (s, 3H).

Second, the azide was reduced to the amine. ¹H NMR (300 MHz, CDCl₃) δ 7.06 (s, 1H), 7.01-6.92 (m, 2H), 3.83-3.70 (m, 3H), 2.92-2.72 (m, 3H), 2.47 (s, 2H), 1.85-1.69 (m, 2H), 1.48-1.33 (m, 1H), 0.90 (s, 9H).

### Step 5: TERT-BUTYL (1S,2R)-1-(3,5-DIFLUOROBENZYL)-2-HYDROXY-3-{[(1 S)-2-(HYDROXYMETHYL)-7-NEOPENTYL-1,2,3,4-TETRAHYDRONAPHTHALEN-1-YL]AMINO}PROPYLCARBAMATE

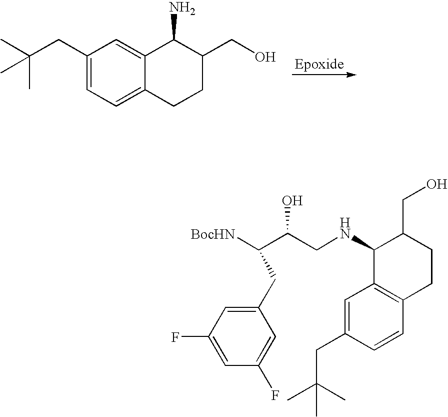

The coupling was performed essentially according to the method of preparing tert-butyl (1S,2R)-1-(3,5-difluorobenzyl)-3-(3,4-dihydro-2H-chroman-4-ylamino)-2-hydroxypropylcarbamate in step 3 of Example 50 below: An IPA (15 mL) solution of tert-butyl (1S)-2-(3,5-difluorophenyl)-1-[(2S)-oxiran-2-yl]ethylcarbamate (0.54 g, 1.8 mmol) and 3,4-dihydro-2H-chroman-4-ylamine (0.40 g, 2.6 mmol) was heated at 60° C. (oil bath) with stirring overnight. The IPA was removed in vacuo and the residue dissolved in EtOAc and washed with 1 N HCl. The organic layer was dried (magnesium sulfate) and concentrated in vacuo to yield 0.75 g of the desired product as a mixture of epimers. HRMS (ESI+) calc'd for C₂₄H₃₀N₂O₄F₂ m/z 449.2252 [M+H]⁺.

The resulting crude product was purified by flash chromatography (silica gel, 1-10% methanol/methylene chloride). ¹H NMR (300 MHz, CDCl₃) δ 7.01-6.91 (m, 3H), 6.76-6.60 (m, 5H), 4.62 (d, J=8.9 Hz, 1H), 4.34-4.30 (m, 1H), 4.07-3.89 (m, 2H), 3.83-3.61 (m, 4H), 3.53-3.47 (m, 2H), 2.95-2.86 (m, 2H), 2.80-2.63 (m, 3H), 2.59-2.57 (m, 2H), 2.45 (s, 2H), 2.15-2.05 (m, 1H), 1.81-1.77 (m, 1H), 1.36 (s, 9H), 0.89 (s, 9H).

### Step 6: PREPARATION OF N-((1S,2R)-1-(3,5-DIFLUOROBENZYL)-2-HYDROXY-3-{[(1 S)-2-(HYDROXYMETHYL)-7-NEOPENTYL-1,2,3,4-TETRAHYDRONAPHTHALEN-1-YL]AMINO)PROPYL)ACETAMIDE

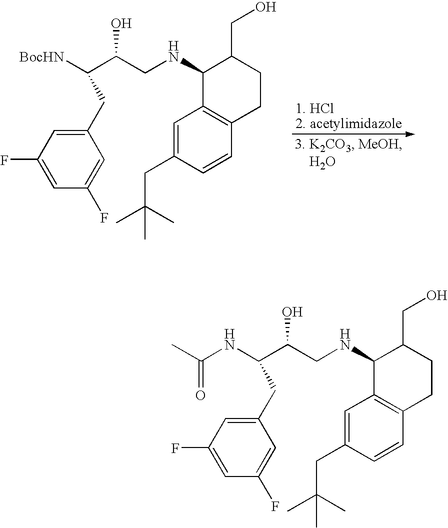

The above compound was prepared essentially according to the method of preparing N-{(1S,2R)-1-(3,5-difluorobenzyl)-2-hydroxy-3-[(6-iodo-4-methyl-3,4-dihydro-2H-chromen-4-yl)amino]propyl}acetamide in Example 70, step 5: tert-butyl (1S,2R)-1-(3,5-difluorobenzyl)-2-hydroxy-3-[(6-iodo-3,4-dihydro-2H-chroman-4-yl)amino]propylcarbamate (3.0 g, 5.2 mmol) was dissolved in 30 mL of 25% TFA/CH₂Cl₂ and stirred at room temperature for 30 min. The mixture was diluted with CH₂Cl₂, washed with NaHCO₃and brine, and dried (sodium sulfate). The solvent was removed in vacuo and the resulting residue dissolved in CH₂Cl₂ (52 mL). The mixture was chilled to 0° C. Et₃N (1. mL, 11.9 mmol) and acetyl imidazole (0.68 g, 6.2 mmol) were added and the mixture was allowed to warm to room temperature over night. The CH₂Cl₂ was removed in vacuo and the residue dissolved in EtOAc, washed with 1N HCl, NaHCO₃ (1×30 mL), and brine, dried (sodium sulfate), and conc. in vacuo to yield 2.5 g (92%) of the title compound as a light yellow solid. MS (ESI+) for C₂₁H₂₃F₂₁N₂O₃ m/z 517.0 [M+H]⁺.

The Boc-protected amine was then deprotected. ESI MS m/z 447 [C₂₆H₃₆F₂N₂O₂+H]⁺.

Next, the amine was acetylated. Then the residue was dissolved in methanol (6 mL) and water (3 mL) and treated with potassium carbonate (300 mg, 2.17 mmol). The reaction mixture was stirred at room temperature for 2 h. The solvent was removed under reduced pressure. The residue was acidified with 1N hydrochloric acid and extracted with ethyl acetate. The combined extracts were washed with saturated sodium chloride, and then dried (sodium sulfate), filtered, and concentrated under reduced pressure. Purification by flash column chromatography (silica gel, 0-5% methanol/methylene chloride) provided the desired product (80 mg, 44%) as a white foam: IR (ATR) 3265, 3072, 2948, 2864, 1626, 1595, 1550, 1459, 1364, 1315, 1115, 1071, 984, 842 cm⁻¹; ¹H NMR (300 MHz, CD₃OD) δ 7.15 (s, 1H), 7.07-7.00 (m, 2H), 6.83-6.72 (m, 3H), 4.18 (d, J=5.9 Hz, 1H), 4.06-3.99 (m, 1H), 3.74-3.64 (m, 2H), 3.57 (t, J=8.4 Hz, 1H), 3.34 (s, 2H), 3.13-3.07 (m, 1H), 2.94-2.59 (m, 5H), 2.49 (s, 2H), 2.30-2.20 (m, 1H), 2.04-1.98 (m, 1H), 1.81 (s, 3H), 1.64-1.57 (m, 1H), 0.91 (s, 9H); ESI MS m/z 489 [C₂₈H₃₈F₂N₂O₃+H]⁺; HPLC (Phenomenex Synergi Max-RP Column, 150×4.6 mm, 4μ; A: H₂O; B: CH₃CN; Gradient: 30-100% B over 15 min; flow 1.0 mL/min; Detection: 220 nm) 98.2% (AUC), t_{R}=9.41 min. Anal. Calc'd for C₂₁H₂₄F₂N₂O₄.H₂O: C, 66.38; H, 7.96; N, 5.53; Found: C, 66.18; H, 7.80; N, 5.45.

### EXAMPLE 9

### PREPARATION OF 5-[((1S,2R)-1-(3,5-DIFLUOROBENZYL)-3-{[(1 S)-7-ETHYL-1,2,3,4-TETRAHYDRONAPHTHALEN-1-YL]AMINO}-2-HYDROXYPROPYL)AMINO]-5-OXOPENTANOIC ACID

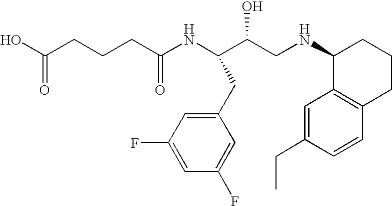

Glutaric anhydride (0.073 g, 0.64 mmol) was added to a solution of 3-amino-4-(3,5-difluoro-phenyl)-1-(7-ethyl-1,2,3,4-tetrahydro-naphthalen-1-ylamino)-butan-2-ol (0.240 g, 0.64 mmol), triethylamine (0.268 mL, 1.92 mmol), and chloroform (3 mL) and the reaction was stirred overnight at 60° C. Reaction was washed with 1N HCl, 10% NaHCO₃, brine, dried (magnesium sulfate), filtered, and concentrated in vacuo to give 5-[((1 S,2R)-1-(3,5-difluorobenzyl)-3-{[(1S)-7-ethyl-1,2,3,4-tetrahydronaphthalen-1-yl]amino}-2-hydroxypropyl)amino]-5-oxopentanoic acid (100 mg), which was purified via prep-HPLC. ¹H NMR (400 MHz, CD₃OD) δ 1.26 (t, J=8 Hz, 3 H), 1.73 (m, 2 H), 1.89 (m, 1 H), 2.01 (m, 1 H), 2.17 (m, 6 H), 2.68 (d, J=8 Hz, 2 H), 2.93 (d, J=6 Hz, 1 H), 3.02 (m, 1 H), 3.30 (m, 2 H), 3.88 (m, 1 H), 4.09 (m, 1 H), 4.57 (m, 1 H), 6.79 (m, 1 H), 6.88 (m, 3 H), 6.93 (d, J=6 Hz, 1 H), 7.20 (m, 2 H), 7.31 (s, 1 H); OAMS: ES+488.9 ES−486.9.

### EXAMPLE 10

### PREPARATION OF 4-[((1S,2R)-1-(3,5-DIFLUOROBENZYL)-3-{[(1 S)-7-ETHYL-1,2,3,4-TETRAHYDRONAPHTHALEN-1-YL]AMINO}-2-HYDROXYPROPYL)AMINO]-4-OXOBUTANOIC ACID

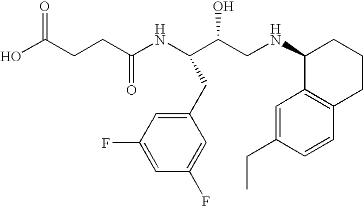

The above compound was prepared essentially according to the method of Example 9. The crude product was purified via prep-HPLC. ¹H NMR (400 MHz, CD₃OD) δ 1.27 (t, J=8 Hz, 3 H), 1.88 (m, 1 H), 2.04 (m, 1 H), 2.25 (m, 3 H), 2.48 (m, 2 H), 2.70 (m, 4 H), 2.81 (m, 1 H), 2.93 (m, 1 H), 3.12 (dd, J=8, 13 Hz, 1 H), 3.32 (m, 2 H), 3.87 (m, 1 H), 4.04 (m, 1 H), 4.51 (s, 1 H), 6.80 (m, 1 H), 6.86 (d, J=6 Hz, 2 H), 7.18 (dd, J=8,19 Hz, 2 H), 7.32 (s, 1 H); OAMS: ES+474.9, ES−472.9.

### EXAMPLE 11

### PREPARATION OF N-((1S,2R)-1-(3,5-DIFLUOROBENZYL)-3-{[(1S)-7-ETHYL-1,2,3,4-TETRAHYDRONAPHTHALEN-1-YL]AMINO}-2-HYDROXYPROPYL)ETHANETHIOAMIDE HYDROCHLORIDE

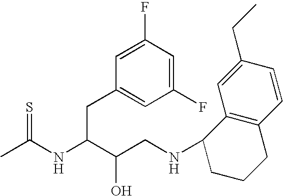

Following essentially the procedure described below, (2R,3S)-3-amino-4-(3,5-difluorophenyl)-1-{[(1S)-7-ethyl-1,2,3,4-tetrahydronaphthalen-1-yl]amino}butan-2-ol dihydrochloride (0.71 mmol) was converted to N-((1S,2R)-1-(3,5-difluorobenzyl)-3-{[(1 S)-7-ethyl-1,2,3,4-tetrahydronaphthalen-1-yl]amino}-2-hydroxypropyl)ethanethioamide hydrochloride (158 mg, 0.34 mmol, 47%), which was obtained as a white solid: ¹H NMR {CDCl₃) δ 9.5 (br s, 1 H), 9.1 (d, 1 H), 7.95 (br, 1 H), 7.39 (s, 1 H), 7.15-7.07 (m, 2 H), 6.73 (m, 2 H), 6.60 (m, 1 H), 4.77 (m, 1 H), 4.47 (m, 1 H), 4.34 (m, 1 H), 3.0 (d, J=7 Hz, 2 H), 2.97 (m, 1 H), 2.73 (m, 3 H), 2.61 (q, J=7.5 Hz, 2 H), 2.53 (s, 3 H), 2.15 (m, 1 H), 2.02 (m, 1 H), 1.87 (m, 1 H), 1.79 (m, 1 H), 1.23 (t, J=7.5 Hz, 3 H); IR (diffuse reflectance) 3194, 3029, 2964, 2932, 2872, 1627, 1597, 1459, 1439, 1420, 1384, 1153, 1119, 982, 847 cm⁻¹. MS (CI) m/z (rel intensity) 433 (MH+,24), 221 (36), 184 (51), 176 (27), 174 (49), 172 (99), 159 (49), 156 (27), 77 (31), 60 (27), 58 (52). HRMS (ESI) calc'd for C₂₄H₃₀N₂OSF₂+H₁ 433.2125, found 433.2114. Anal. Calc'd for C₂₄H₃₀F₂N₂OS.HCl+H₂O: C, 59.19; H, 6.83; N, 5.75; Cl, 7.28; S, 6.58; Found: C, 59.84; H, 6.70; N, 5.88; Cl, 6.91; S, 6.40.

Analogous procedure: N-((1S,2R)-1-(3,5-difluorobenzyl)-2-hydroxy-3-{[1-(3-isopropylphenyl)cyclohexyl]amino)propyl)ethanethioamide hydrochloride.

### Step 1: PREPARATION OF THIOACETYL-N-PHTHALIMIDE

Thioacetamide (1.9 g, 25 mmol) was suspended in 40 mL of CH₂Cl₂ and cooled in an ice bath under N₂(g). Phthaloyldichloride (3.6 mL, 25 mmol) was added slowly over 10 min via syringe while the mixture was stirred. The mixture became a clear orange solution transiently, eventually depositing a precipitate. After stirring for 40 h, the mixture was concentrated in vacuo. The oily coral solid was triturated with hexanes, yielding a precipitate, which was filtered off to yield 0.2 g of a light coral solid: ¹H NMR (CDCl₃) δ 7.99 (m, 2 H), 7.86 (m, 2 H), 3.08 (s, 3 H). The residual solids remaining after trituration with hexanes were further triturated with ether and then with CH₂Cl₂. The combined mother liquors were concentrated to yield about 3 g of a red oily solid, which was chromatographed over silica gel (10% to 20% ethyl acetate in heptane. The red fractions contained a product (concentrated to a coral solid, 0.77 g) with the same TLC retention (R_{f}=0.32, 20% ethyl acetate in heptane) as the coral solid that precipitated from hexanes. The total recovery is 0.97 g, 4.7 mmol, 19%.

### Step 2: PREPARATION OF N-((1S,2R)-1-(3,5-DIFLUOROBENZYL)-2-HYDROXY-3-{[1-(3-ISOPROPYLPHENYL) CYCLOHEXYL]AMINO}PROPYL) ETHANETHIOAMIDE HYDROCHLORIDE

Solid thioacetyl-N-phthalimide (80 mg, 0.39 mmol) was added to a solution of the free base of N-{1-(3,5-difluoro-benzyl)-2-hydroxy-3-[1-(3-isopropyl-phenyl)-cyclohexylamino]-propyl}-acetamide (164 mg, 0.39 mmol) in 3 mL of approximately 0° C. CH₂Cl₂ under N₂(g). The mixture was stirred for 20 min, and then 3 mL of methanol and 3 mL of 1N NaOH were added. The mixture was taken up in ethyl acetate and washed with 1N NaOH, water, and brine, dried (sodium sulfate), concentrated, and chromatographed over silica gel (4% methanol (containing 2% NH₄OH) in CH₂Cl₂). Product-containing fractions were concentrated to a colorless oil, which is dissolved in ether and treated with ethereal HCl. Concentration yielded 97 mg (0.19 mmol, 49%) of N-((1S,2R)-1-(3,5-difluorobenzyl)-2-hydroxy-3-{[1-(3-isopropylphenyl) cyclohexyl]amino}propyl)ethanethioamide hydrochloride as a white solid: ¹H NMR (CDCl₃+CD₃OD drop) δ 7.42-7.37 (m, 2 H), 7.29 (m, 2 H), 6.73 (m, 2 H), 6.62 (m, 2 H), 4.67 (m, 1 H), 4.10 (m, 1 H), 3.11 (dd, J=5, 14 Hz, 1 H), 2.96 (hept, J=7 Hz, 1 H), 2.83 (m, 1 H), 2.65-2.4 (m, 4 H, obscured by solvent), 2.38 (s, 3 H), 2.07 (m, 2 H), 1.78 (m, 2 H), 1.59 (m, 1 H), 1.44-1.35 (m, 3 H), 1.28 (d, J=7 Hz, 6 H); MS (CI) m/z 475.3 [M+H]⁺.

### EXAMPLE 12

### PREPARATION OF N-((1S,2R)-1-(3,5-DIFLUOROBENZYL)-3-{[(1S)-7-ETHYL-1,2,3,4-TETRAHYDRONAPHTHALEN-1-YL]AMINO}-2-HYDROXYPROPYL)-2,2-DIFLUOROACETAMIDE HYDROCHLORIDE

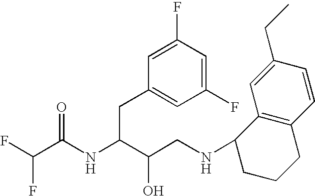

Using methods analogous to those previously described, (2R,3S)-3-amino-4-(3,5-difluorophenyl)-1-{[(1S)-7-ethyl-1,2,3,4-tetrahydronaphthalen-1-yl]amino}butan-2-ol dihydrochloride (0.33 mmol) was converted to N-((1S,2R)-1-(3,5-difluorobenzyl)-3-{[(1S)-7-ethyl-1,2,3,4-tetrahydronaphthalen-1-yl]amino}-2-hydroxypropyl)-2,2-difluoroacetamide hydrochloride (88 mg, 0.18 mmol, 54%), which was obtained as a white solid: ¹H NMR (CDCl₃) δ 7.36 (s, 1 H), 7.12 (m, 2 H), 6.71 (m, 2 H), 6.64 (m, 1 H), 5.81 (t, J=54 Hz, 1 H), 4.46 (m, 1 H), 4.18 (m, 1 H), 4.07 (m, 1 H), 3.12 (m, 2 H), 2.77 (m, 4 H), 2.63 (q, J=7.5 Hz, 2 H), 2.2 (m, 1 H), 2.05 (m, 1 H), 1.96 (m, 1 H), 1.86 (m, 1 H), 1.23 (t, J=7.5 Hz, 3 H); MS (CI) m/z 453.5 [M+H]⁺.

### EXAMPLE 13

### PREPARATION OF N-((1S,2R)-1-(3,5-DIFLUOROBENZYL)-3-{[(1S)-7-ETHYL-1,2,3,4-TETRAHYDRONAPHTHALEN-1-YL]AMINO}-2-HYDROXYPROPYL)-2-FLUOROACETAMIDE HYDROCHLORIDE

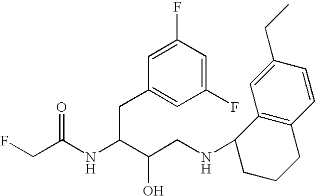

Using methods analogous to those previously described, (2R,3S)-3-amino-4-(3,5-difluorophenyl)-1-{[(1 S)-7-ethyl-1,2,3,4-tetrahydronaphthalen-1-yl]amino}butan-2-ol dihydrochloride (0.0.71 mmol) was converted to N-((1S,2R)-1-(3,5-difluorobenzyl)-3-{[(1S)-7-ethyl-1,2,3,4-tetrahydronaphthalen-1-yl]amino}-2-hydroxypropyl)-2-fluoroacetamide hydrochloride (248 mg, 0.53 mmol, 74%), which was obtained as a white solid: ¹H NMR (CDCl₃) δ 9.85 (br, 1 H), 8.41 (br, 1 H), 7.45 (s, 1 H), 7.09 (m, 2 H), 6.97 (d, J=8.6 Hz, 1 H), 6.68 (m, 2 H), 6.62 (m, 1 H), 4.70 (dq; J˜50, 11 Hz, 2 H), 4.48 (m, 1 H), 4.29 (m, 1 H), 4.16 (m, 1 H), 3.1-3.0 (m, 2 H), 2.83-2.69 (m, 4 H), 2.59 (q, J=7.5 Hz, 2 H), 2.21 (m, 1 H), 2.02 (m, 2 H), 1.78 (m, 1 H), 1.21 (t, J=7.5 Hz, 3 H); MS (CI) m/z 435.3 [M+H]⁺.

### EXAMPLE 14

### PREPARATION OF (1S,2R)-1-(3,5-DIFLUOROBENZYL)-3-{[(1S)-7-ETHYL-1,2,3,4-TETRAHYDRONAPHTHALEN-1-YL]AMINO}-2-HYDROXYPROPYLFORMAMIDE

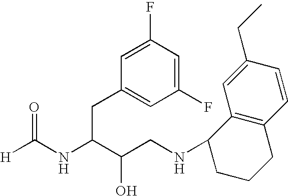

Using methods analogous to those previously described, but without making the HCl salt, (2R,3S)-3-amino-4-(3,5-difluorophenyl)-1-{[(1S)-7-ethyl-1,2,3,4-tetrahydronaphthalen-1-yl]amino}butan-2-ol dihydrochloride (0.0.31 mmol) was converted to (1S,2R)-1-(3,5-difluorobenzyl)-3-{[(1S)-7-ethyl-1,2,3,4-tetrahydronaphthalen-1-yl]amino}-2-hydroxypropylformamide (70 mg, 0.17 mmol, 56%), which was obtained as a white solid. ¹H NMR (CDCl₃) δ 8.11 (s, 1 H), 7.16 (s, 1 H), 7.03 (s, 2 H), 6.76 (m, 2 H), 6.67 (m, 1 H), 5.83 (d, J=9 Hz, 1 H), 4.25 (m, 1 H), 3.74 (m, 1 H), 3.53 (m, 1 H), 3.03 (dd, J=4.8, 14.4 Hz, 1 H), 2.90-2.69 (m, 5 H), 2.61 (q, J=7.6 Hz, 2 H), 1.85 (m, 3 H), 1.76 (m, 1 H), 1.23 (t, J=7.6 Hz, 3 H); MS (CI) m/z 403.3 [M+H]⁺. A trace NMR doublet (J=11.8 Hz) appears at δ 7.73, tentatively attributed to an intramolecularly cyclized form of the product in the deuterochloroform solution.

### EXAMPLE 15

### Preparation of Additional Compounds

The following compounds are synthesized in a manner analogous to treating a sample of the starting amine in of dichloromethane is with triethylamine; adding a solution of methanesulfonyl chloride in dichloromethane; stirring the solution overnight; evaporating the solvent and isolating the product by reverse-phase HPLC; substituting methanesulfonyl chloride with various reagents: N-[(1S, 2R)-1-(3,5-Difluorobenzyl)-3-((1S)-7-ethyl-1,2,3,4-tetrahydro-naphthalen-1-ylamino)-2-hydroxypropyl]-2-hydroxy-acetamide, N-[(1S, 2R)-1-(3,5-Difluorobenzyl)-3-((1S)-7-ethyl-1,2,3,4-tetrahydro-naphthalen-1-ylamino)-2-hydroxy-propyl]-2-methoxy-acetamide, 2-(2-Butoxy-ethoxy)-N-[(1S, 2R)-1-(3,5-difluorobenzyl)-3-((1S)-7-ethyl-1,2,3,4-tetrahydro-naphthalen-1-ylamino)-2-hydroxy-propyl]-acetamide, N-[(1S, 2R)-1-(3,5-Difluorobenzyl)-3-((1S)-7-ethyl-1,2,3,4-tetrahydro-naphthalen-1-ylamino)-2-hydroxy-propyl]-2-(2-oxo-cyclopentyl)-acetamide, 2,2-Dichloro-N-[(1S, 2R)-1-(3,5-difluoro-benzyl)-3-((1S)-7-ethyl-1,2,3,4-tetrahydro-naphthalen-1-ylamino)-2-hydroxy-propyl]-acetamide, 2-Chloro-N-[(1S, 2R)-1-(3,5-difluoro-benzyl)-3-((1S)-7-ethyl-1,2,3,4-tetrahydro-naphthalen-1-ylamino)-2-hydroxy-propyl]-acetamide, and 2-Bromo-N-[(1S, 2R)-1-(3,5-difluoro-benzyl)-3-((1S)-7-ethyl-1,2,3,4-tetrahydro-naphthalen-1-ylamino)-2-hydroxy-propyl]-acetamide.

### EXAMPLE 16

### PREPARATION OF N-[(1S, 2R)-1-(3,5-DIFLUOROBENZYL)-3-((1S)-7-ETHYL-1,2,3,4-TETRAHYDRO-NAPHTHALEN-1-YLAMINO)-2-HYDROXY-PROPYL]-METHANESULFONAMIDE

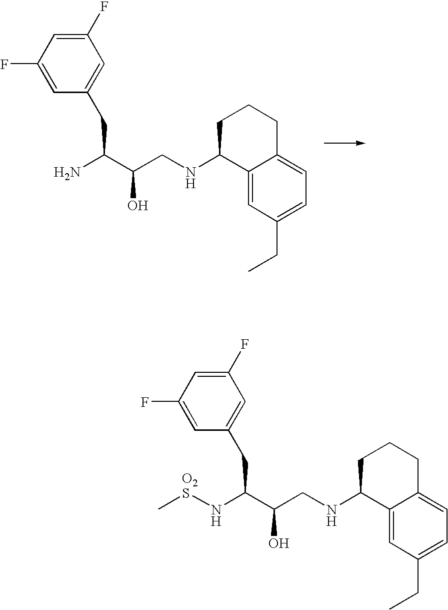

A 30 mg sample of the starting amine in 1 mL of dichloromethane was treated with 33 μL of triethylamine. A solution of 6 μL of methanesulfonyl chloride in 0.5 mL of dichloromethane was added and the solution was stirred overnight. The solvent was evaporated and the product was isolated by reverse-phase HPLC. Mass spectroscopy yielded m/z=453.2.

### EXAMPLE 17

### PREPARATION OF N-(1S, 2R)-{1-(3,5-DIFLUORO-BENZYL)-3-[(1S)-7-(2,2-DIMETHYLPROPYL)-1,2,3,4-TETRAHYDRO-NAPHTHALEN-1-YLAMINO]-2-HYDROXYPROPYL}-ACETAMIDE

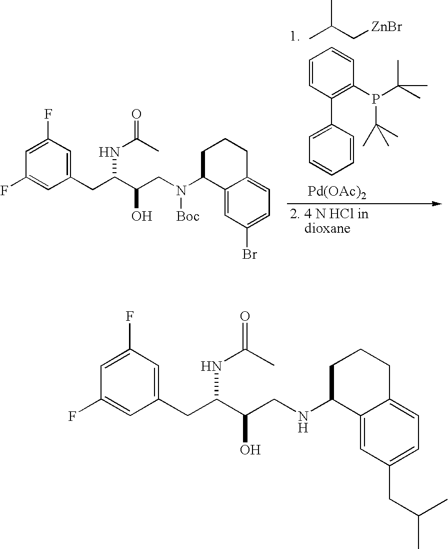

Palladium(II) acetate (0.2 equiv, 0.07 mmol, 15.8 mg) and 2-(di-t-butylphosphino)biphenyl (0.1 equiv, 0.035 mmol, 10.5 mg) were dissolved in THF (2 mL) and deoxygenated with a subsurface N₂(g) purge for 5 min. The bromide (1 equiv, 0.352 mmol, 200 mg) was then added to this solution as a solid, followed by isobutyl zinc bromide (0.5 M solution in THF, 3 equiv, 1.1 mmol, 2.1 mL). The reaction was stirred overnight at room temperature under N₂(g). After 12 h, the reaction was partitioned between EtOAc and H₂O, and extracted into EtOAc. The combined organic extracts were washed with brine, dried (sodium sulfate), filtered and concentrated. Column chromatography on SiO₂ with (30 to 50% EtOAc in hexanes) yielded the pure desired Boc protected product (148 mg, 77% yield). MS (CI) m/z567.2 [M+Na]⁺.

Removal of the Boc group was achieved by dissolving the above compound in 4N HCl in dioxane (1 mL) and stirring at room temperature for 1 h under N₂(g). The resulting white cloudy mixture was concentrated to give the final product. (100 mg, 85% yield) ¹HNMR (CD₃OD): δ 7.3 (s, 1H), 7.15 (s, 2H), 6.9 (m, 2H), 6.8 (m, 1H), 4.6 (t, 1H), 4.05 (m, 1H), 3.9 (m, 1H), 3.2 (m, 2H), 3.0 (m, 1H, 2.8 (m, 2H), 2.7 (m, 2H), 2.5 (d, 2H), 2.2 (m, 2H), 2.0 (m, 1H), 1.85 (s, 3H), 1.85, m, 1 H), 0.9 (m, 6H). MS (CI) m/z 445.2 [M+H]⁺.

### EXAMPLE 18

### PREPARATION OF N-(1S, 2R)-{1-(3,5-DIFLUORO-BENZYL)-3-[(1S-7-(2,2-DIMETHYLPROPYL)-1,2,3,4-TETRAHYDRO-NAPHTHALEN-1-YLAMINO]-2-HYDROXYPROPYL}-ACETAMIDE

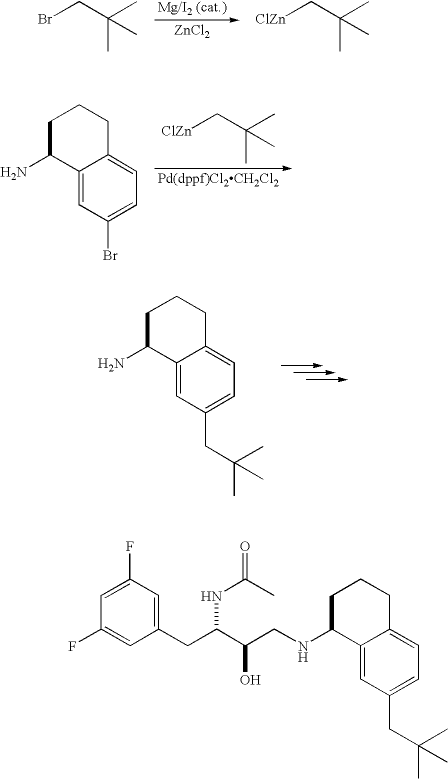

The neopentyl zinc was prepared according to the procedure in Tetrahedron Letters, 1983, vol. 24, pp. 3823-24.

The crude neopentylzinc chloride suspension (3 equiv, 24 mmol, 48 mL), followed by Pd(dppf)Cl₂.CH₂Cl₂ (0.05 equiv, 0.4 mmol, 330 mg) was added to the bromotetralin amine (1 equiv, 8 mmol, 1.71 g). The reaction was stirred at room temperature under N₂(g) overnight. The suspension quickly turned yellow and eventually turned purplish overnight. After 12 h, the reaction was quenched with NH₄Cl (aq) and extracted 3× with EtOAc. The combined organic extracts were washed with brine, dried (sodium sulfate), filtered, and concentrated. Column chromatography on SiO₂ (2 to 10% MeOH in CH₂Cl₂) yielded the desired neopentyl tetralin amine. (1.5 g, 86% yield) ¹HNMR (CDCl₃): δ 7.15 (s, 1H), 6.95 (m, 2H), 3.95 (m, 1H), 2.8 (m, 2H), 2.4 (s, 2H), 2.0 (m, 2H), 1.7 (m, 2H), 1.6 (broad s, 2H), 1.0 (s, 9H); MS (CI) m/z 201.2 [M−NH₂]⁺.

The final compound was synthesized via epoxide opening, protecting group deprotection, and acetylation as previously described: MS (CI) m/z 459.2 [M+H]⁺.

### EXAMPLE 19

### PREPARATION OF N-{1-(3,5-DIFLUORO-BENZYL)-3-[7-(2,2-DIMETHYL-PROPYL)-5-ETHYL-1,2,3,4-TETRAHYDRO-NAPHTHALEN-1-YLAMINO]-2-HYDROXY-PROPYL}-ACETAMIDE

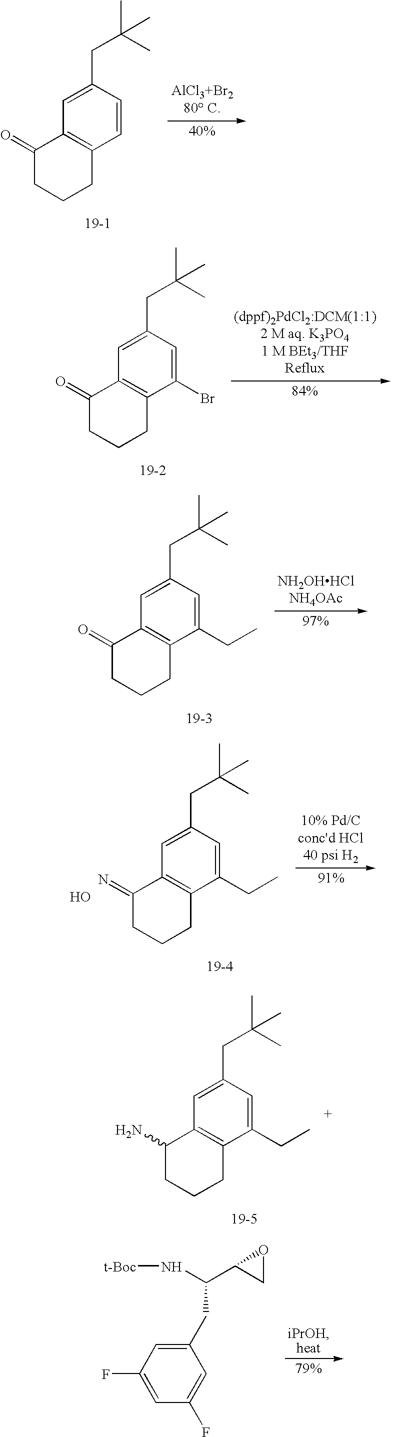

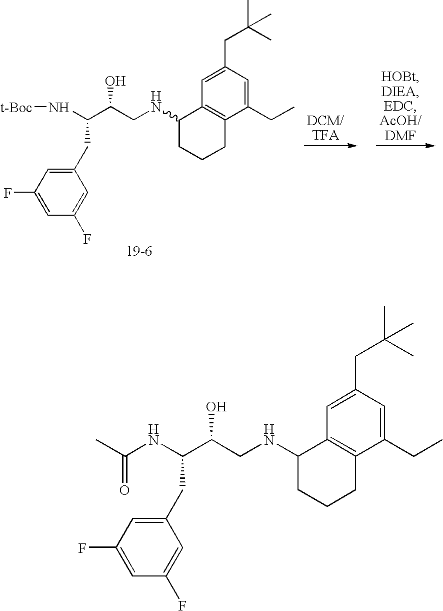

### Step 1

The conversion of compound 19-1 to compound 19-2 was performed essentially according to the method of Example 27, below. The resulting crude product was purified by flash column chromatography to yielded compound 19-2 as a solid: TLC (10% EtOAc/Hexane) R_{f}=0.48; MS (CI) m/z 295.0 [M+H]⁺.

### Step 2

Palladium-mediated transfer of the ethyl group onto the aryl bromide was described previously to give compound 19-3: Yield: 84%; MS (CI) m/z 245.2 [M+H]⁺.

### Step 3

Formation of the oxime was performed as previously described in Example 1 and Example 3, step 4 to give compound 19-4. Yield: 97%; MS (CI) m/z 260.2 [M+H]⁺.

### Step 4

Reduction of the oxime to the amine was achieved as previously described in Example 1 and Example 3, step 5 to give compound 19-5: yield: 91%; MS (CI) m/z 229.2 [M+H]⁺.

### Step 5

Epoxide opening was performed as previously described in Example 1 and Example 3, step 6: yield: 79%; MS (CI) m/Vz 545.3 [M+H]⁺.

### Step 6

Boc deprotection and acetylation was performed as described in, for example, Example 3, step 7 and below. The resultant diastereomeric mixture was purified by reverse-phase HPLC to give both isomers of N-(1S,2R)-{1-(3,5-Difluorobenzyl)-3-[7-(2,2-dimethylpropyl)-5-ethyl-1,2,3,4-tetrahydronaphthalen-1-ylamino]-2-hydroxypropyl)acetamide. MS (CI) m/z 487.3 [M+H]⁺.

### EXAMPLE 20

### PREPARATION OF [3-ACETYLAMINO-4-(3,5-DIFLUORO-PHENYL)-2-HYDROXY-BUTYL]-(5-BROMO-1,2,3,4-TETRAHYDRO-NAPHTHALEN-1-YL)-CARBAMIC ACID TERT-BUTYL ESTER

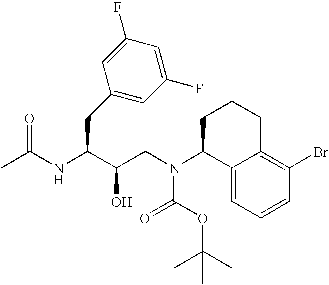

### Step 1: EPOXIDE OPENING WITH (S)-7-BROMO-1-AMINOTETRALIN (7-BROMO-1,2,3,4-TETRAHYDRO-NAPHTHALEN-1-YLAMINE)

The above compound was prepared essentially according to the method of Example 50, step 3. The coupled product was crystallized from isopropyl alcohol. LC-MS analysis indicated about 99% purity. LC-MS: [M+H]⁺=527, retention time=2.34 min, Phenomenex Luna C18 (30cm×4.6 mm), 20-70% CH₃CN/water/0.1% trifluoroacetic acid in 2.33 min, flow rate 1.5 mL/min.

### Step 2: DEPROTECTION OF BOC GROUP

The above compound was prepared essentially using the method of preparing S,R 1-(3,5-Difluorobenzyl)-3-[1-(3-Bromophenyl)Cyclopropylamino)]-2-Hydroxypropyl Amine: The Boc-protected amine (13.5 g, 26.7 mmol) was treated with 4N HCl in dioxane (30 mL). Methanol (15 mL) was added and the mixture became homogeneous before depositing a precipitate. The mixture was stirred for 3 h, then the volatiles were removed in vacuo. The residue was taken up in 1N NaOH and extracted with diethyl ether. The combined ether extracts were washed with brine, dried (magnesium sulfate), filtered and evaporated in vacuo to give the desired amine (6.5 g), which was used directly in the next step.

### Step 3: ACYLATION OF N-TERMINAL AMINE

The above compound was prepared essentially using the method of preparing: N-[3-[1-(3-bromo-phenyl)-cyclopropylamino]-1-(3,5-difluoro-benzyl)-2-hydroxy-propyl]-acetamide, which was prepared essentially according to the procedure of preparing N-((1S,2R)-1-(3,5-difluorobenzyl)-2-hydroxy-3-{[(4S)-6-iodo-3,4-dihydro-2H-chroman-4-yl]amino}propyl)-2-hydroxy-2-methylpropanamide: (2R,3S)-3-amino-4-(3,5-difluoro phenyl)-1-{[(4S)-6-iodo-3,4-dihydro-2H-chroman-4-yl]amino}butan-2-ol (1 equiv) was combined with 2-methylacetic acid, (1.25 equiv), EDC (1.5 equiv) and HOBt (1.5 equiv) in DMF/DCM (1:1, 10 mL). The reaction mixture was treated with Et₃N and stirred at room temperature for 6 h. The reaction mixture was poured onto EtOAc and washed with 1M HCl. The organics were dried (magnesium sulfate) and concentrated to give an oil which was purified by reverse phase preparative HPLC. HRMS (ESI+) calc'd for C₂₃H₂₇F₂₁N₂O₄ m/z 561.1063 [M+H]⁺; Found 561.1047.

However, acetic acid was used as the acid. The desired product was obtained as a white solid (11.75 g, 97%). LC-MS analysis indicated a purify of 94%. LC-MS: [M+H]=453, 455, Rt=1.86 min, Phenomenex Luna C18 (30 cm×4.6 mm), 20-70% CH₃CN/water/0.1% trifluoroacetic acid in 2.33 min, flow rate 1.5 mL/min.

LC-MS analysis indicated a purity of 99%. LC-MS: [M+H]=467, 469, retention time=1.94 min, Phenomenex Luna C18 (30cm×4.6 mm), 20-70% CH₃CN/water/0.1% trifluoroacetic acid in 2.33 min, flow rate 1.5 mL/min.

### Step 4: ADDING BOC GROUP

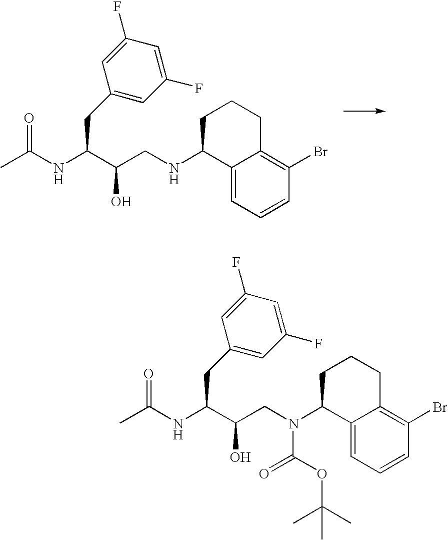

The starting compound (7.80 g, 16.7 mmol) was dissolved in dichloromethane (150 mL). Di-tert-butyldicarbonate (3.82g, 17.5 mmol) was added and the mixture was stirred for 3 days. The mixture was then concentrated in vacuo and the residue passed through a pad of silica gel (eluted 1 L 2:1 hexane/ethyl acetate, 0.5 L 5% MeOH/dichloromethane) to give the desired product (8.52 g, 90%).

LC-MS analysis indicated a purity of 99%. LC-MS: [M+Na]=589, 591, retention time=5.12 min, Phenomenex Luna C18 (30cm×4.6 mm), 20-70% CH₃CN/water/0.1% trifluoroacetic acid in 2.33 min, flow rate 1.5 mL/min.

### EXAMPLE 21

### PREPARATION OF N-(1S, 2R)-[1-(3,5-DIFLUORO-BENZYL)-2-HYDROXY-3-[(1S)-(7-ISOBUTYL-1,2,3,4-TETRAHYDRO NAPHTHALEN-1-YLAMINO)]-PROPYL]-ACETAMIDE

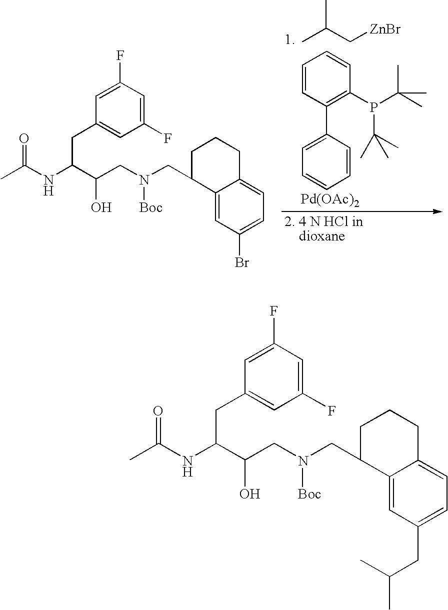

Palladium(II) acetate (0.2 equiv, 0.07 mmol, 15.8 mg) and 2-(di-t-butylphosphino)biphenyl (0.1 equiv, 0.035 mmol, 10.5 mg) were dissolved in THF (2 mL) and deoxygenated with a subsurface N₂(g) purge for 5 min. The bromide (1 equiv, 0.352 mmol, 200 mg) was then added to this solution as a solid, followed by isobutyl zinc bromide (0.5 M solution in THF, 3 equiv, 1.1 mmol, 2.1 mL). The reaction was stirred overnight at room temperature under N₂(g). After 12 h, the reaction was partitioned between EtOAc and H₂O, and extracted 3× into EtOAc. The combined organic extracts were washed with brine and dried over sodium sulfate, filtered, and concentrated. Column chromatography on SiO₂ using a 30 to 50% gradient of EtOAc in hexanes yielded the pure desired Boc protected product. (148 mg, 77% yield) MS (CI) m/z 567.2 [M+Na]⁺.

Removal of the Boc group was achieved by dissolving the above compound in 4N HCl in dioxane (1 mL) and stirring at room temperature for 1 h under N₂(g). The resulting white cloudy mixture was concentrated to give the final product. (100 mg, 85% yield) ¹HNMR (CD₃OD): δ 7.3 (s, 1H), 7.15 (s, 2H), 6.9 (m, 2H), 6.8 (m, 1H), 4.6 (t, 1H), 4.05 (m, 1H), 3.9 (m, 1H), 3.2 (m, 2H), 3.0 (m, 1H, 2.8 (m, 2H), 2.7 (m, 2H), 2.5 (d, 2H), 2.2 (m, 2H), 2.0 (m, 1H), 1.85 (s, 3H), 1.85, m, 1 H), 0.9 (m, 6H). MS (CI) m/z 445.2 [M+H]⁺.

### EXAMPLE 22

### SYNTHESIS OF N-{1-(3,5-DIFLUORO-BENZYL)-3-[7-(2,2-DIMETHYL-PROPYL)-1,2,3,4-TETRAHYDRO-NAPHTHALEN-1-YLAMINO]-2-HYDROXY-PROPYL)-2-FLUORO-ACETAMIDE

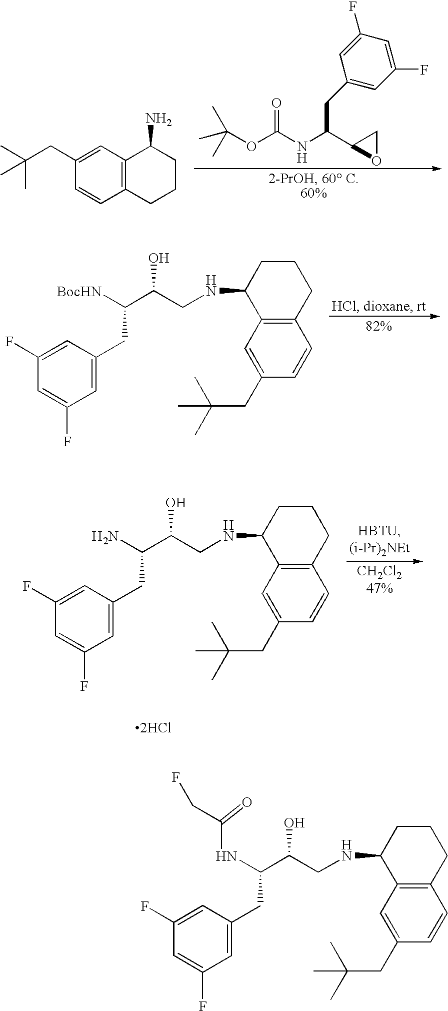

Coupling the enantiomerically pure tetralin amine of 7-(2,2-dimethyl-propyl)-1,2,3,4-tetrahydro-naphthalen-1-ylamine with (1S)-2-(3,5-difluorophenyl)-1-[(2S)-oxiran-2-yl]ethylcarbamate followed by Boc-deprotection and HBTU-mediated acylation yielded the final compound (N-{1-(3,5-difluoro-benzyl)-3-[7-(2,2-dimethyl-propyl)-1,2,3,4-tetrahydro-naphthalen-1-ylamino]-2-hydroxy-propyl}-2-fluoro-acetamide), as predominantly one diastereoisomer.

### Step 1

tert-Butyl (1S)-2-(3,5-difluorophenyl)-1-[(2S)-oxiran-2-yl]ethylcarbamate (0.31 g, 1.03 mmol) was added to a solution of 7-(2,2-dimethyl-propyl)-1,2,3,4-tetrahydro-naphthalen-1-ylamine (0.22 g, 1.03 mmol) in 2-propanol (10 mL) and the reaction mixture was heated to 50° C. for 17 h. The reaction mixture was cooled to room temperature, and the solvent removed under reduced pressure. The resulting residue was partitioned between methylene chloride (20 mL) and water (20 mL). The aqueous phase was extracted with methylene chloride, and the organic phase was washed successively with 0.5 N hydrochloric acid, saturated sodium bicarbonate and sodium chloride (10 mL), dried (sodium sulfate), filtered, and concentrated under reduced pressure. The crude product was purified by flash chromatography (silica, 95:5 methylene chloride/methanol) to yield {1-(3,5-difluoro-benzyl)-3-[7-(2,2-dimethyl-propyl)-1,2,3,4-tetrahydro-naphthalen-1-ylamino]-2-hydroxy-propyl}-carbamic acid tert-butyl ester (0.32 g, 60%) which was carried on without further characterization: ESI MS m/z 517 [C₃₀H₄₂F₂N₂O₃+H].

### Step 2

Hydrogen chloride (1.50 mL, 4 M solution in dioxane, 6.18 mmol) was added to a solution of {1-(3,5-difluoro-benzyl)-3-[7-(2,2-dimethyl-propyl)-1,2,3,4-tetrahydro-naphthalen-1-ylamino]-2-hydroxy-propyl}-carbamic acid tert-butyl ester (0.32 g, 0.61 mmol) in dioxane (5 mL) at room temperature and the reaction mixture stirred for 17 h. The reaction mixture was concentrated under reduced pressure and the resulting residue triturated with diethyl ether to yield 3-amino-4-(3,5-difluoro-phenyl)-1-[7-(2,2-dimethyl-propyl)-1,2,3,4-tetrahydro-naphthalen-1-ylamino]-butan-2-ol HCl (0.25 g, 85%) as a white solid, which was carried on without further purification or characterization: ESI MS m/z 417 [C₂₅H₃₆Cl₂F₂N₂O+H].

### Step 3

A solution of 3-amino-4-(3,5-difluoro-phenyl)-1-[7-(2,2-dimethyl-propyl)-1,2,3,4-tetrahydro-naphthalen-1-ylamino]-butan-2-ol HCl (0.23 g, 0.47 mmol) and N,N-diisopropylethylamine (0.15 mL, 0.94 mmol) in methylene chloride (2 mL) were added to a suspension of sodium fluoroacetate (0.04 g, 0.82 mmol), N,N-diisopropylethylamine (0.23 mL, 1.41 mmol) and HBTU (0.17 g, 0.47 mmol) in methylene chloride (2 mL). The combined mixture was stirred at room temperature for 24 h. Water (20 mL) was added and the aqueous phase was extracted with additional methylene chloride (5 mL). The combined organic phase was washed successively with 0.5 N hydrochloric acid (10 mL) and saturated sodium chloride (10 mL), dried (sodium sulfate), filtered and concentrated under reduced pressure. Purification by preparative HPLC (Phenomenex Luna C18(2) Column, 150×4.6 mm, 5μ; A: 0.05% TFA in 95:5 H₂O/CH₃CN; B: 0.05% TFA in 5:95 H₂O/CH₃CN; Gradient: 30-100% B over 15 min; flow 1.0 mL/min; Detection: 254 nm) yielded N-{1-(3,5-Difluoro-benzyl)-3-[7-(2,2-dimethyl-propyl)-1,2,3,4-tetrahydro-naphthalen-1-ylamino]-2-hydroxy-propyl}-2-fluoro-acetamide (106 mg, 47%) as a white solid: IR (ATR) 3324, 2957, 1659, 1594 cm⁻¹; ¹H NMR (300 MHz, CDCl₃) δ 7.29 (s, 1H), 7.12-6.95 (m, 2H), 6.82-6.55 (m, 4H), 4.93-4.83 (m, 1H), 4.81-4.67 (m, 1H), 4.27-4.18 (m, 1H), 3.75-3.74 (m, 1H), 3.57-3.52 (m,.1H), 3.10-3.04 (m, 1H), 2.94-2.67 (m, 5H), 2.48 (s, 2H), 1.98-1.75 (m, 4H), 1.60-1.40 (br s, 2H), 0.93 (s, 9H); ESI MS m/z 476 [C₂₇H₃₅F₃N₂O₂+H]; HPLC (Phenomenex Synergi Max-RP Column, 150×4.6 mm, 4μ; A: H₂O; B: CH₃CN; Gradient: 30-100% B over 15 min; flow 1.0 mL/min; Detection: 220 nm)>99% (AUC), t_{R}=8.60 min.

### EXAMPLE 23

### The General Scheme Below Can Be Used to Synthesize the Compounds Disclosed and Described in Example 23A and is Not Limiting to the Scope of the Invention

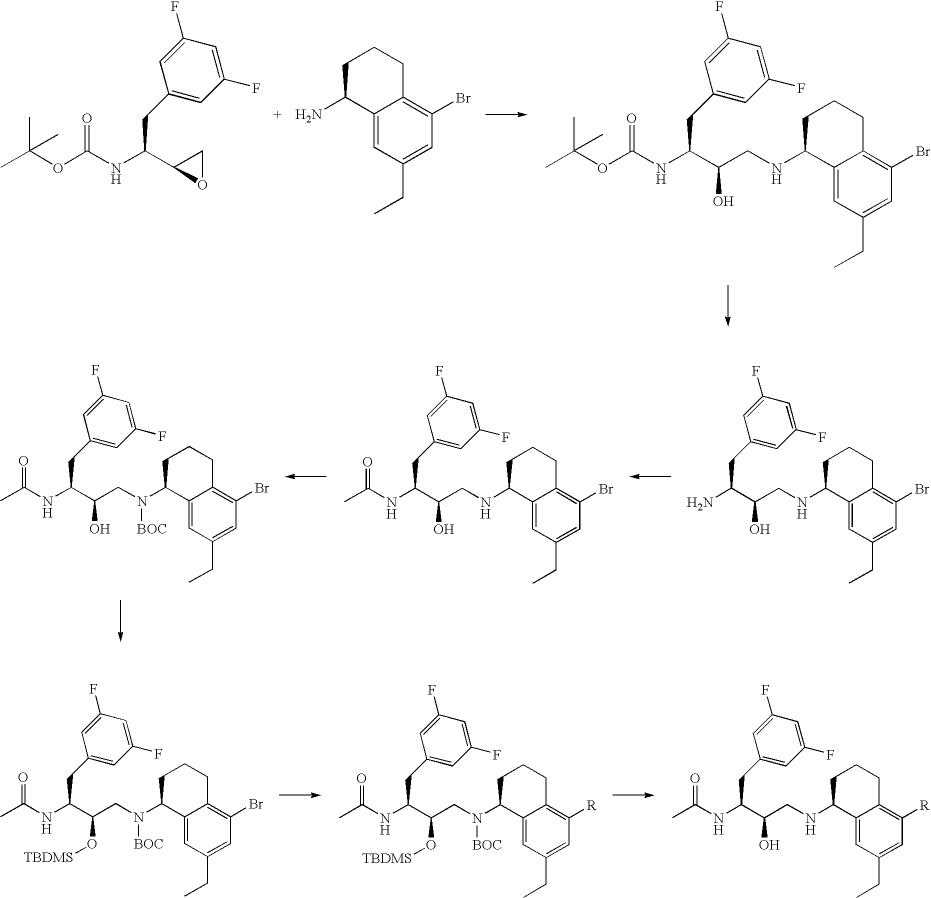

### EXAMPLE 23A

### SYNTHESIS OF N-[(1S, 2R)-3-((1S)-5-BUTYL-7-ETHYL-1,2,3,4-TETRAHYDRO-NAPHTHALEN-1-YLAMINO)-1-(3,5-DIFLUOROBENZYL)-2-HYDROXY-PROPYL]-ACETAMIDE

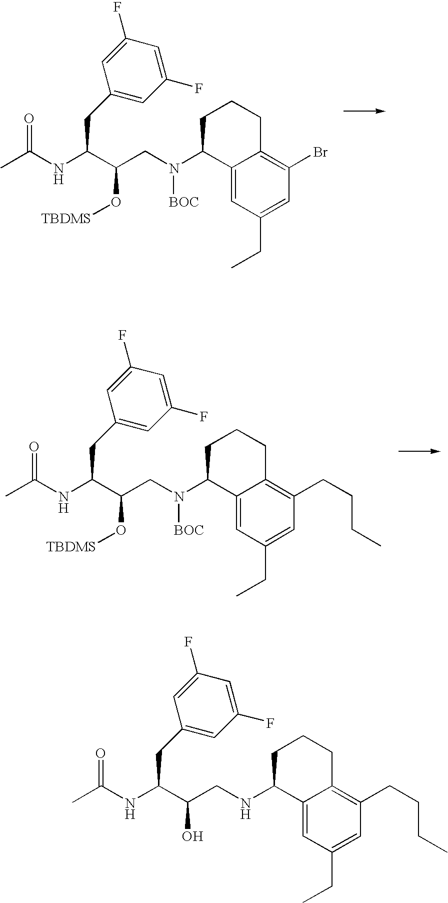

### Step 1: Preparation of [(1S, 2R)-3-((1S)-5-Bromo-7-ethyl-1,2,3,4-tetrahydro-naphthalen-1-ylamino)-1-(3,5-difluoro-benzyl)-2-hydroxypropyl]-carbamic acid tert-butyl ester

A solution of N-Boc-epoxide [2-(3,5-difluoro-phenyl)-1-oxiranyl-ethyl]-carbamic acid tert-butyl ester (869 mg, 2.91 mmol) and 5-bromo-7-ethyl-1,2,3,4-tetrahydro-naphthalen-1-ylamine (783 mg, 2.91 mmol) in 10 mL isopropanol, was heated to 80° C. for 6 h. After completion of the reaction, the mixture was cooled and [3-(5-bromo-7-ethyl-1,2,3,4-tetrahydro-naphthalen-1-ylamino)-1-(3,5-difluoro-benzyl)-2-hydroxy-propyl]-carbamic acid tert-butyl ester crystallized from the crude solution, and was collected by filtration. The crystals were washed with cold ethanol. After vacuum was applied to remove traces of volatiles, the reaction yielded about 995 mg of [(1S, 2R)-3-((1S)-5-bromo-7-ethyl-1,2,3,4-tetrahydro-naphthalen-1-ylamino)-1-(3,5-difluoro-benzyl)-2-hydroxypropyl]-carbamic acid tert-butyl ester ([M+H]⁺=552.8).

### Step 2: Preparation of (3S, 2R)-3-Amino-1-((1S)-5-bromo-7-ethyl-1,2,3,4-tetrahydro-naphthalen-1-ylamino)-4-(3,5-difluoro-phenyl)-butan-2-ol

[(1S, 2R)-3-((1S)-5-Bromo-7-ethyl-1,2,3,4-tetrahydro-naphthalen-1-ylamino)-1-(3,5-difluoro-benzyl)-2-hydroxypropyl]-carbamic acid tert-butyl ester (995 mg) was dissolved in 10 mL of anhydrous CH₂Cl₂, and 10 mL of trifluoroacetic acid (anhydrous) was added. The solution was allowed to stand for 90 min, then the volatiles are removed with a stream of N₂(g). The compound was desalted by extraction between ethyl acetate (10 mL) and saturated aqueous sodium bicarbonate (20 mL). The ethyl acetate phase was washed with saturated sodium bicarbonate, dried (magnesium sulfate), filtered, and concentrated yielding 865 mg of (3S, 2R)-3-Amino-1-((1S)-5-bromo-7-ethyl-1,2,3,4-tetrahydro-naphthalen-1-ylamino)-4-(3,5-difluoro-phenyl)-butan-2-ol ([M+H]⁺=452.8).

### Step 3: Preparation of N-[(1S, 2R)-3-((1S)-5-Bromo-7-ethyl-1,2,3,4-tetrahydro-naphthalen-1-ylamino)-1-(3,5-difluorobenzyl)-2-hydroxypropyl]-acetamide

HOBt (125. mg, 0.93 mmol), N-methyl-morpholine (0.17 mL, 1.55 mmol), and glacial acetic acid (46.4 mg, 0.773) were added to a solution of diamine (3S, 2R)-3-Amino-1-((1S)-5-bromo-7-ethyl-1,2,3,4-tetrahydro-naphthalen-1-ylamino)-4-(3,5-difluoro-phenyl)-butan-2-ol (350 mg, 0.77 mmol) in 5 mL anhydrous CH₂Cl₂. This solution was cooled to 0° C. and solid EDC-HCl (1-[3-(dimethylamino)propyl]-3-ethylcarbodiimide hydrochloride, 163 mg, 0.85 mmol) and a stir bar were added. The reaction is stirred at 0° C. for 12 h. After the warming to room temperature, the solvent is removed with a stream of N₂(g), and the residue washed between ethyl acetate and aqueous saturated sodium bicarbonate. The ethyl acetate phase was dried (magnesium sulfate), filtered, and concentrated by rotory evaporation and high vacuum to yield 295 mg of compound N-[(1S, 2R)-3-((1S)-5-Bromo-7-ethyl-1,2,3,4-tetrahydro-naphthalen-1-ylamino)-1-(3,5-difluorobenzyl)-2-hydroxypropyl]-acetamide ([M+H]⁺=494.8).

### Step 4: Preparation of [(3S, 2R)-3-Acetylamino-4-(3,5-difluorophenyl)-2-hydroxybutyl]-((1S)-bromo-7-ethyl-1,2,3,4-tetrahydronaphthalen-1-yl)-carbamic acid tert-butyl ester

N,N′-diisopropylethylamine (0.35 mL, 1.2 mmol) and di-t-butyl dicarbonate (145 mg, 0.66 mmol) were added to a solution of N-[(1S, 2R)-3-((1S)-5-Bromo-7-ethyl-1,2,3,4-tetrahydro-naphthalen-1-ylamino)-1-(3,5-difluorobenzyl)-2-hydroxypropyl]-acetamide (295 mg, 0.6 mmol) in 5 mL anhydrous THF. The solution was stirred overnight and then the solvent was removed with N₂(g). The residue was partitioned between ethyl acetate (10 mL) and 1N sodium bisulfate (20 mL), washed against aqueous saturated sodium bicarbonate, dried (magnesium sulfate), filtered, then concentrated by rotory evaporation and high vacuum to yield 354.4 mg of [(3S, 2R)-3-Acetylamino-4-(3,5-difluorophenyl)-2-hydroxybutyl]-((1S)-5-bromo-7-ethyl-1,2,3,4-tetrahydronaphthalen-1-yl)-carbamic acid tert-butyl ester ([M+H]⁺=594.5).

### Step 5. Preparation of [(1S, 2R)-3-Acetylamino-2-(tert-butyl-dimethyl-silanyloxy)-4-(3,5-difluorophenyl)-butyl]-((1S)-5-bromo-7-ethyl-1,2,3,4-tetrahydro-naphthalen-1-yl)-carbamic acid tert-butyl ester

[(3S, 2R)-3-Acetylamino-4-(3,5-difluorophenyl)-2-hydroxybutyl]-((1S)-5-bromo-7-ethyl-1,2,3,4-tetrahydronaphthalen-1-yl)-carbamic acid tert-butyl ester (354 mg, 0.6 mmol) was added to a solution of t-butyidimethylsilyl chloride (1 05 mg, 0.66 mmol) and imidazole (102 mg, 1.5 mmol) in anhydrous dimethylformamide (3 mL) and the solution was stirred at room temperature for 16 h. DMF was removed via rotory evaporation. The resulting residue was dissolved in ethyl acetate and washed with 1N sodium bisulfate and saturated aqueous sodium bicarbonate, dried (magnesium sulfate), filtered, and concentrated to yield [(1S, 2R)-3-Acetylamino-2-(tert-butyl-dimethyl-silanyloxy)-4-(3,5-difluorophenyl)-butyl]-((1S)-5-bromo-7-ethyl-1,2,3,4-tetrahydro-naphthalen-1-yl)-carbamic acid tert-butyl ester. (M+H=731.2). The product was used in step 6 without further purification.

### Step 6. Preparation of [(1S, 2R)-3-acetylamino-2-(tert-butyidimethylsilanyloxy)-4-(3,5-difluorophenyl)-butyl]-((1S)-5-butyl-7-ethyl-1,2,3,4-tetrahydro-naphthalen-1-yl)-carbamic acid tert-butyl ester

The following reaction was performed under N₂(g). A solution of Pd(OAc)₂ (2.25 mg, 0.01 mmol) and 2-(di-t-butylphosphino)biphenyl (5.9 mg, 0.01 mmol) in 0.1 mL of anhydrous THF was added to a solution of [((1S, 2R)-3-Acetylamino-2-(tert-butyl-dimethyl-silanyloxy)-4-(3,5-difluorophenyl)-butyl]-((1S)-5-bromo-7-ethyl-1,2,3,4-tetrahydro-naphthalen-1-yl)-carbamic acid tert-butyl ester (73 mg, 0.1 mmol) in 0.1 mL of anhydrous THF. Butylzinc bromide (0.5M in THF, 0.5 mL, 0.25 mmol) was then added to the reaction mixture, which was stirred for 16 h, then the solvent was removed with N₂(g), and then the residue was dissolved in methanol (1 mL) for purification by reverse phase HPLC. The butylated product [(1S, 2R)-3-acetylamino-2-(tert-butyidimethylsilanyloxy)-4-(3,5-difluorophenyl)-butyl]-((1S)-5-butyl-7-ethyl-1,2,3,4-tetrahydro-naphthalen-1-yl)-carbamic acid tert-butyl ester ([M+H]⁺=709.1) was concentrated and obtained as an oil.

### Step 7. Preparation of N-[(1S, 2R)-3-((1S)-5-Butyl-7-ethyl-1,2,3,4-tetrahydro-naphthalen-1-ylamino)-1-(3,5-difluorobenzyl)-2-hydroxypropyl]-acetamide

Anhydrous trifluoracetic acid (1 mL) was added to a solution of [(1S, 2R)-3-acetylamino-2-(tert-butyldimethylsilanyloxy)-4-(3,5-difluorophenyl)-butyl]-((1S)-5-butyl-7-ethyl-1,2,3,4-tetrahydro-naphthalen-1-yl)-carbamic acid tert-butyl ester in 1 mL of CH₂Cl₂. After 1 hr, the reaction mixture was concentrated to yield N-[(1S, 2R)-3-((1S)-5-butyl-7-ethyl-1,2,3,4-tetrahydro-naphthalen-1-ylamino)-1-(3,5-difluorobenzyl)-2-hydroxypropyl]-acetamide ([M+H]⁺=472.8).

### EXAMPLE 24

### GENERAL SYNTHESIS FOR N-(1S, 2R)-[1-(3,5-DIFLUORO-BENZYL)-2-HYDROXY-3-(1S)-(1,2,3,4-TETRAHYDRO-NAPHTHALEN-1-YLAMINO)-PROPYL]-ACETAMIDE

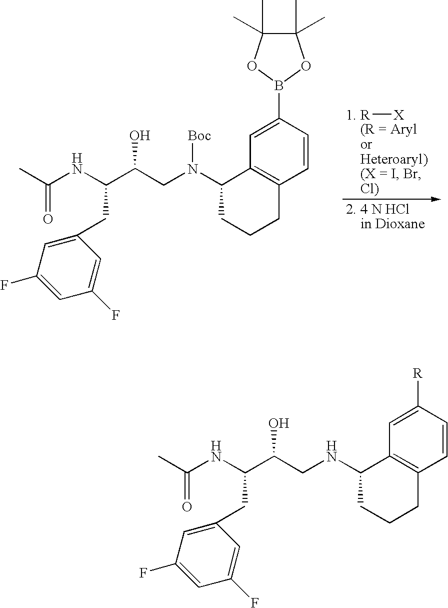

### EXAMPLE 25

### GENERAL SYNTHESIS FOR N-(1S, 2R)-[1-(3,5-DIFLUORO-BENZYL)-3-((1S)-7-FURAN-3-YL-1,2,3,4-TETRAHYDRO-NAPHTHALEN-1-YLAMINO)-2-HYDROXY-PROPYL]-ACETAMIDE

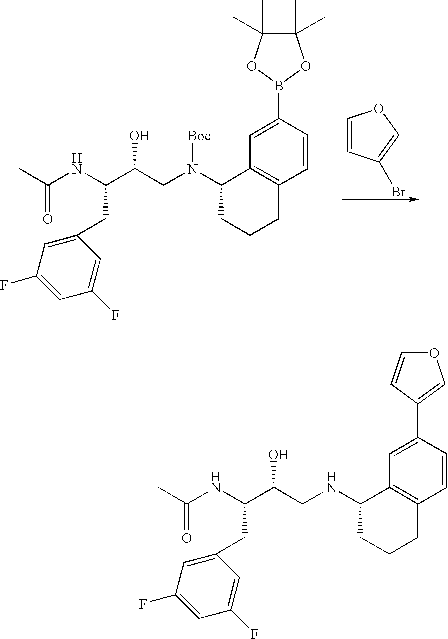

3-Bromofuran (4.85 mg, 0.033 mM) and tetrakis(triphenyl-phosphine)palladium [0] (3.81 mg, 10 mol. wt %) were dissolved in 300 μL 1,2-dimethoxyethane (glyme) (DME). 99 μL 2M Na₂CO₃ in dH₂O was added to the reaction mixture. N-(1S, 2R)-[3-Acetylamino-4-(3,5-difluoro-phenyl)-2-hydroxy-butyl]-[7-(4,4,5,5-tetramethyl-[1,3,2]dioxaborolan-2-yl)-(1S)-1,2,3,4-tetrahydro-naphthalen-1-yl]-carbamic acid tert-butyl ester (20.28 mg, 0.033 mM) was added and the reaction was stirred at 90° C. overnight. The reaction mixture was concentrated and dissolved in 1.5 mL methanol. The reaction mixture was purified by HPLC and concentrated. The product was dissolved in 500 μL 4N HCl in dioxane and allowed to stand at room temperature for 30 min. The reaction mixture was then concentrated to yield the title compound. MS (ESI+) for C₂₆H₂₈F₂N₂O₃ m/z 455.2 [M+H]⁺.

### EXAMPLE 26

### General Procedure for the Preparation of Representative Compounds

Various compounds can be synthesized with the appropriate reagents using the same procedure as that for N-(1S, 2R)-[1-(3,5-Difluoro-benzyl)-3-((1S)-7-furan-3-yl-1,2,3,4-tetrahydro-naphthalen-1-ylamino)-2-hydroxy-propyl]-acetamide in Example 25: [(1S, 2R)-3-acetylamino-2-(tert-butyldimethylsilanyloxy)4-(3,5-difluorophenyl)-butyl]-((1S)-5-butyl-7-ethyl-1,2,3,4-tetrahydro-naphthalen-1-yl)-carbamic acid tert-butyl ester were prepared from [(1S, 2R)-3-Acetylamino-2-(tert-butyl-dimethyl-silanyloxy)-4-(3,5-difluorophenyl)-butyl]-((1S)-5-bromo-7-ethyl-1,2,3,4-tetrahydro-naphthalen-1-yl)-carbamic acid tert-butyl ester according to the procedure for preparing [(1S, 2R)-3-acetylamino-2-(tert-butyldimethylsilanyloxy)-4-(3,5-difluorophenyl)-butyl]-((1S)-5-butyl-7-ethyl-1,2,3,4-tetrahydro-naphthalen-1-yl)-carbamic acid tert-butyl ester (above), except that the butylzinc bromide used in the preparation of [(1S, 2R)-3-acetylamino-2-(tert-butyldimethylsilanyloxy)-4-(3,5-difluorophenyl)-butyl]-((1S)-5-butyl-7-ethyl-1,2,3,4-tetrahydro-naphthalen-1-yl)-carbamic acid tert-butyl ester was replaced with other zinc reagents. The protecting groups were removed from the intermediate compounds [(1S, 2R)-3-acetylamino-2-(tert-butydimethylsilanyloxy)-4-(3,5-difluorophenyl)-butyl]-((1S)-5-butyl-7-ethyl-1,2,3,4-tetrahydro-naphthalen-1-yl)-carbamic acid tert-butyl ester as described for the preparation of N-[(1S, 2R)-3-((1S)-5-butyl-7-ethyl-1,2,3,4-tetrahydro-naphthalen-1-ylamino)-1-(3,5-difluorobenzyl)-2-hydroxypropyl]-acetamide from [(1S, 2R)-3-acetylamino-2-(tert-butyidimethylsilanyloxy)-4-(3,5-difluorophenyl)-butyl]-((1S)-5-butyl-7-ethyl-1,2,3,4-tetrahydro-naphthalen-1-yl)-carbamic acid tert-butyl ester.

### EXAMPLES 27-34

### General Precursor Synthesis

### EXAMPLE 27

### PREPARATION OF 7-BROMO-1-TETRALONE (7-BROMO-3,4-DIHYDRO-2H-NAPHTHALEN-1-ONE)

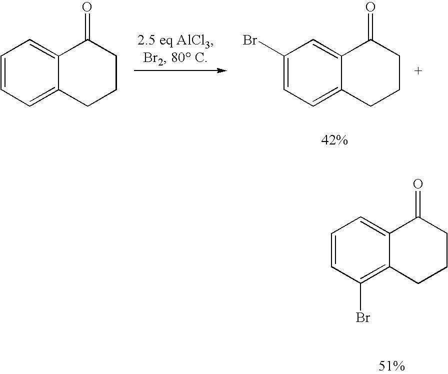

7-Bromo-1-tetralone was prepared according to the procedure described in Cornelius, L. A. M.; Combs, D. W. Synthetic Communications, 1994, 24, 2777-2788. The above isomers were separated using silica gel flash chromatography (Biotage Flash 75, 20:1 hexanes:MTBE) to yield 5-bromo-1-tetralone (11.59 g, 51%) and 7-bromo-1-tetralone (9.45 g, 42%).

Tetralin-1-ol compounds may be prepared as shown in Example 28 below. For example, (R)-7-ethyltetralin-1-ol was prepared in three steps starting from 7-ethyl-1-tetralone. The first step involves an asymmetric reduction of the ketone using borane and Corey's oxazaboralidine chiral auxiliary. This reduction produced a 97:3 mixture of (presumably) R/S enantiomers. A Mitsunobu-like Sn2 conversion to the azide and LiAlH₄ reduction to the amine produced material 98:2 S/R.

### EXAMPLE 28

### PREPARATION OF (R)-7-ETHYLTETRALIN-1-OL (7-ETHYL-1,2,3,4-TETRAHYDRO-NAPHTHALEN-1-OL)

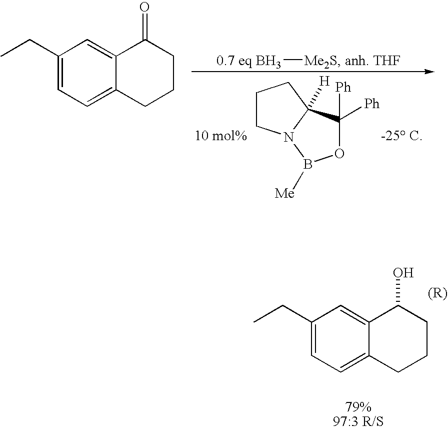

See generally: Jones, T. K. et al., J. Org. Chem., 1991, 56, 763-769. For example, 7-ethyl-1-tetralone (2.29 g, 13.1 mmol) was dissolved in anhydrous THF (40 mL). Activated 4 Å molecular sieves were added and the mixture was aged for 2 h before transferring via cannula to a 250 mL three-necked round bottom flask fitted with a dropping funnel, thermometer, and a nitrogen inlet. The solution was cooled to −25° C. and 1M (S)-tetrahydro-1-methyl-3,3-diphenyl-1H,3H-pyrollo[1,2-c][1,3,2]oxazaborole in toluene (1.3 mL, 1.3 mmol) was added. The source of the oxazoborole was Aldrich, cat. no. 45,770-1, “(S)-2-methyl-CBS-oxazaborolidine”. Use of the S-auxiliary will produce R-alcohols. The use of 5 mol % oxazaborolidine catalyst in the reaction described above should give comparable results.

The dropping funnel was charged with a solution of borane-methylsulfide (0.70 g, 0.87 mL, 9.3 mmol) in anhydrous THF (15 mL, dried over 4 Å sieves). The borane solution was added dropwise over 20 min, keeping the reaction temperature less than −20° C. The mixture was stirred for 1 h at −15 to −20° C., then the reaction was quenched by careful addition of methanol (15 mL) at −20° C. and allowed to warm to room temperature, then stirred for 16 h. The volatiles were removed in vacuo and the residue was purified by silica gel chromatography (Biotage Flash 65, 6:1 hexanes:ethyl acetate) to yield (R)-7-ethyltetralin-1-ol (1.82 g, 79%). Analytical chiral HPLC indicated a 96.6/3.4 mixture of enantiomers (Chirocel OD-H column, isocratic elution 2:98 IPA/hexane, 0.9 mL/min, room temperature 15.2 min (minor enantiomer), 17.5 min (major enantiomer)).

### EXAMPLE 29

### PREPARATION OF (S)-7-ETHYL-1,2,3,4-TETRAHYDRO-1-NAPHTHYLAMINE HYDROCHLORIDE

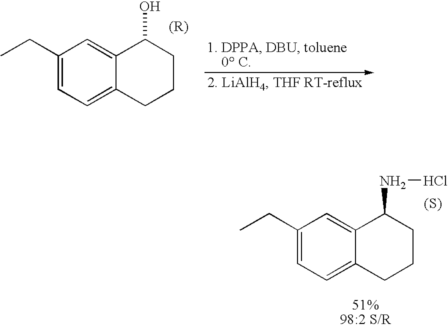

See generally: Rover, S. et al., F. M. J. Med. Chem., 2000, 43,1329-1338. The authors therein report a somewhat diminished yield due to partial formation of a dihydronapthalene via elimination of the hydroxyl moiety.

Specifically, a solution of (R)-7-ethyltetralin-1-ol (1.77 g, 10.1 mmol) in toluene (25 mL) was cooled in an ice bath and treated with diphenylphosphorylazide (DPPA, 3.3 g, 2.7 mL, 12 mmol). A solution of 1,8-diazabicyclo[5.4.0]undec-7-ene (DBU, 1.8 g, 1.8 mL, 12 mmol) in toluene (8 mL) was added over 20 min and the mixture was allowed to stir at 0° C. for 2 h and room temperature for 16 h. The mixture was filtered through a pad of silica gel (eluted 6:1 hexanes/ethyl acetate) to remove precipitates and the volatiles were removed in vacuo to give an oily residue of the crude S-azide. This material was used directly in the next step without further characterization.

The azide was dissolved in dry THF (20 mL) and added dropwise at room temperature to a slurry of lithium aluminum hydride (0.459 g, 12 mmol) in dry THF (20 mL). The mixture was stirred at room temperature for 1 h and then heated to reflux for 1 h. The reaction was cooled to room temperature and quenched by successive addition of water (0.45 mL), 15% aq NaOH (0.45 mL) and water (1.4 mL). The resulting mixture was stirred for 1 h and then filtered through a pad of Celite® (eluted diethyl ether). The volatiles were removed in vacuo and the residue taken up into ethyl acetate (40 mL) and treated with 4N HCl in dioxane (3 mL). The resulting precipitate was filtered (wash ethyl acetate), collected, and vacuum dried to give (S)-7-ethyl-1,2,3,4-tetrahydro-1-napthylamine hydrochloride as a white solid (1.09 g, 51%). Analytical chiral HPLC indicated a 96:4 mixture of enantiomers (Daicel Crownpak (−) column, isocratic elution 10% methanol in water (0.1% TFA), 0.8 mL/min, room temperature, 56.2 min (minor enantiomer), 78.2 min (major enantiomer).)

### EXAMPLE 30

### PREPARATION OF (R)-7-BROMOTETRALIN-1-OL (7-BROMO-1,2,3,4-TETRAHYDRO-NAPHTHALEN-1-OL)

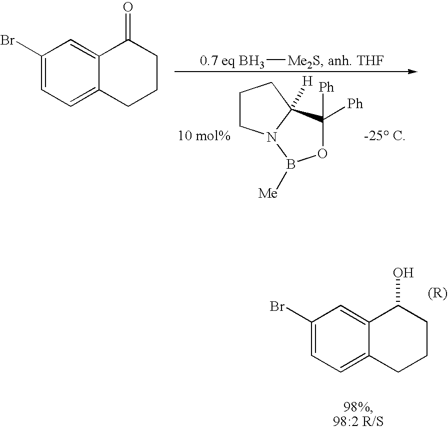

Reduction of 7-bromo-tetral-1-one is performed using the general procedure described in Example 28. Analytical chiral HPLC of the product indicated a 98:2 mixture of enantiomers (Chirocel OD-H column, isocratic elution 2:98 IPA/hexane, 0.9 mL/min, room temperature 18.4 min (minor enantiomer), 19.5 min (major enantiomer).) ¹H NMR was consistent with that previously reported for the racemate: Saito, M. et al., J. Med. Chem., 1980, 23,1364-1372.

### EXAMPLE 31

### PREPARATION OF (S)-7-BROMO-1,2,3,4-TETRAHYDRO-1-NAPTHYLAMINE HYDROCHLORIDE

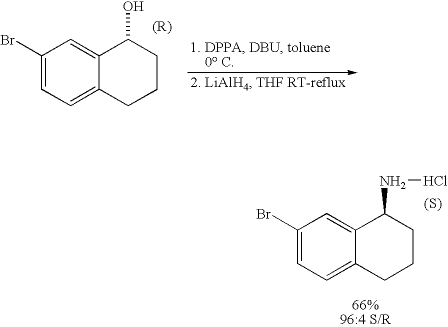

The above compound is prepared essentially according to the procedure described in Example 29. The final compound is obtained as a white solid. Analytical chiral HPLC indicated a 96:4 mixture of enantiomers (Daicel Crownpak (−) column, isocratic elution 10% methanol in water (0.1% TFA), 0.8 mumin, retention time 39.4 min (minor enantiomer), 57.6 min (major enantiomer)).

### EXAMPLE 32

### PREPARATION OF 5-BROMO-7-ETHYL-1,2,3,4-TETRAHYDRO-NAPHTHALEN-1-YLAMINE

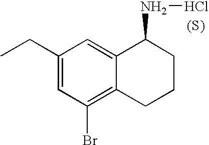

### Step 1: PREPARATION OF 5-BROMO-7-ETHYL-1-TETRALONE

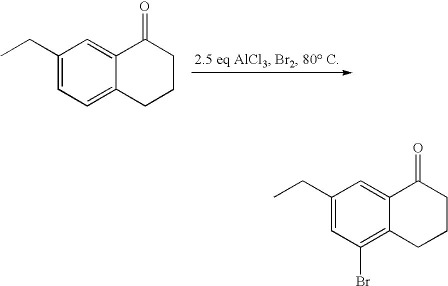

The bromination was performed essentially according to the procedure of Cornelius, L. A. M., Combs, D. W., Synthetic Communications 1994, 24, 2777-2788. The product was separated using silica gel flash chromatography (Biotage Flash 75, 10:1 hexanes:MTBE) to yield the purified product (7.4 g, 75%).

LC-MS analysis indicated the presence of a dibromo product co-eluting with desired product. This material was taken on to the next step and separated.

### Step 2: PREPARATION OF (R)-7-ETHYL-5-BROMOTETRALIN-1-OL (5-BROMO-7-ETHYL-1,2,3,4-TETRAHYDRO-NAPHTHALEN-1-OL)

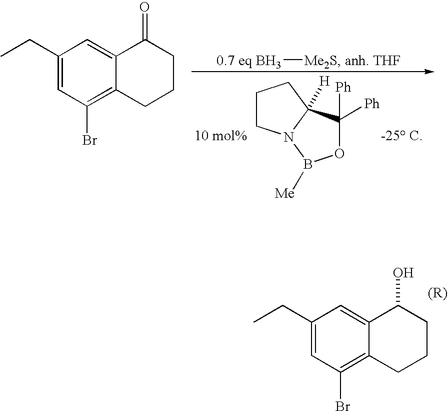

The above product was prepared essentially according to the method of Example 28. The resulting product was purified by silica gel chromatography (Biotage Flash 65, 10/1 hexanes/ethyl acetate) to yield (R)-7-ethyl-5-bromotetralin-1-ol (4.0 g, 53%).

### Step 3: PREPARATION OF (S)-7-ETHYL-5-BROMO-1,2,3,4-TETRAHYDRO-1-NAPTHYLAMINE HYDROCHLORIDE

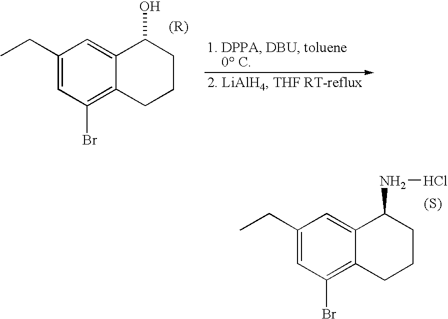

The above compound was prepared essentially according to the method of Example 29. First the azide was prepared. Second, the azide was reduced with lithium aluminum hydride to yield the product as a white solid. LC-MS: [M−NH2]=237, 239, retention time=6.34 min, Phenomenex Luna C18 (30 cm×4.6 mm), 5-20% CH₃CN/water/0.1% trifluoroacetic acid in 3.33 min, flow rate 1.5 mL/min.

### EXAMPLE 33

### Synthesis of a Chiral Amine

The starting material, which is readily available, was protected and then underwent palladium-mediated coupling with neopentylzinc chloride (generated in situ) to give neopentyl substituted tetraline protected by Boc (Rₐ). Subsequent deprotection of Boc yielded intermediate amine (R_{b}) as its hydrochloride salt, which was utilized in the synthesis of additional targets (infra).

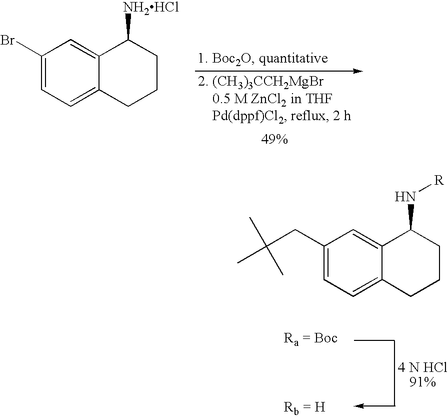

### EXAMPLE 34

### Synthesis of a Tetralone

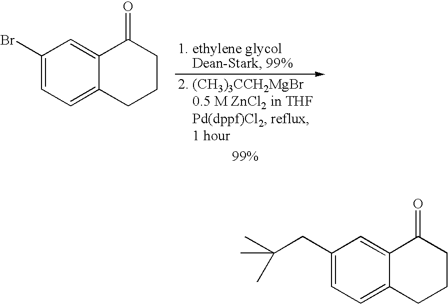

7-Bromotetralone was protected as its dioxolane and then underwent palladium-mediated coupling with neopentylzinc chloride (generated in situ) to yield, after acidic work-up, neopentyl substituted tetralone (7-(neopentyl)-3,4-dihydro-2H-naphthalen-1-one).

### Step 1

A solution of 7-bromotetralone (5.0 g, 22.21 mmol) in benzene (100 mL) containing ethylene glycol (5.0 mL, 88.8 mmol) and p-toluenesulfonic acid monohydrate (420 mg, 2.22 mmol) was heated at reflux in a Dean-Stark apparatus for 24 h. The reaction mixture was cooled to room temperature, concentrated under reduced pressure, and the resulting residue partitioned between ethyl acetate and water. The phases were separated, and the organic phase was washed with saturated sodium chloride, dried (sodium sulfate), filtered, and concentrated under reduced pressure to yield the desired dioxolane (5.97 g, 99%) as a golden oil: ¹H NMR (300 MHz, CDCl₃) δ 7.57 (d, J=2.0 Hz, 1H), 7.32 (dd, J=8.2, 2.0 Hz, 1H), 6.96 (d, J=8.2 Hz, 1H), 4.23-4.07 (m, 4H), 2.73-2.72 (m, 2H), 2.04-1.94 (m, 4H).

### Step 2

A solution of the neo-pentylmagnesium bromide prepared above (60 mL) was added dropwise at room temperature over 20 min to a solution of zinc chloride (60 mL, 0.5 M in THF, 30.0 mmol). Following Grignard addition, the reaction mixture was stirred for 0.5 h to yield a white heterogeneous suspension. [1,1′-Bis(diphenylphosphino) ferrocene]dichloropalladium(II) complex with dichloromethane (1:1) (816 mg, 1.0 mmol) was added in one portion followed by dropwise addition of a solution of the dioxolane prepared in step 1 (2.69 g, 10.0 mmol) in THF (10 mL) to yield a yellow reaction mixture. The mixture was then heated at reflux for 1 h to yield a brown solution. The reaction mixture was cooled to room temperature, quenched with 10% hydrochloric acid (100 mL). and was stirred at room temperature overnight. The reaction mixture was diluted with diethyl ether and the phases separated. The organic phase was washed with water, saturated sodium chloride, dried (sodium sulfate), filtered, and concentrated under reduced pressure to yield a black oil. Purification by flash column chromatography (silica, 19:1 hexanes/ethyl acetate) yielded 7-(neopentyl)-3,4-dihydro-2H-naphthalen-1-one (2.17 g, 99%) as a yellow oil: IR (ATR) 3359, 2957, 1762, 1686, 1521, 1236, 1126, 1076, 1053, 1028 cm⁻¹; ¹H NMR (300 MHz, CDCl₃) δ 7.79 (s, 1H), 7.26-7.22 (m, 1H), 7.15 (m, 1H), 2.96-2.92 (m, 2H), 2.67-2.62 (m, 2H), 2.50 (s, 2H), 2.17-2.08 (m, 2H), 0.89 (s, 9H); ESI MS m/z 217 [C₁₅H₂₀O+H]⁺; HPLC: (Phenomenex Luna C18(2) Column, 150×4.6 mm, 4μ; A: 95:5 H₂O/CH₃CN; B: 5:95 H₂O/CH₃CN; Gradient: 40-100% B over 15 min; flow 1.0 mL/min; Detection: 254 nm)>99% (AUC), t_{R}=13.30 min.

### EXAMPLE 35

### Representative Compounds

The following formula (I) compounds can be prepared essentially according to the procedures set forth in the above examples and schemes, as well as those known in the art:
N-[3-(5,7-Diethyl-1,2,3,4-tetrahydronaphthalen-1-ylamino)-1-(3,5-difluorobenzyl)-2-hydroxypropyl]-acetamide, N-[1-(3,5-Difluorobenzyl)-3-(7-ethyl-5-propyl-1,2,3,4-tetrahydro-naphthalen-1-ylamino)-2-hydroxypropyl]-acetamide, N-[1-(3,5-Difluorobenzyl)-3-(7-ethyl-5-isobutyl-1,2,3,4-tetrahydronaphthalen-1-ylamino)-2-hydroxypropyl]-acetamide, N-{1-(3,5-Difluoro-benzyl)-2-hydroxy-3-[7-(4-methyl-thiophen-3-yl)-1,2,3,4-tetrahydro-naphthalen-1-ylamino]-propyl}-acetamide, N-{1-(3,5-Difluoro-benzyl)-2-hydroxy-3-[7-(3-methyl-3H-imidazol-4-yl)-1,2,3,4-tetrahydro-naphthalen-1-ylamino]-propyl}-acetamide, N-[1-(3,5-Difluoro-benzyl)-2-hydroxy-3-(7-pyrimidin-2-yl-1,2,3,4-tetrahydro-naphthalen-1-ylamino)-propyl]-acetamide, N-{1-(3,5-Difluoro-benzyl)-2-hydroxy-3-[7-(4-trifluoromethyl-pyrimidin-2-yl)-1,2,3,4-tetrahydro-naphthalen-1-ylamino]-propyl}-acetamide, N-{1-(3,5-Difluoro-benzyl)-2-hydroxy-3-[7-(2-methylsulfanyl-pyrimidin-4-yl)-1,2,3,4-tetrahydro-naphthalen-1-ylamino]-propyl}-acetamide, N-{1-(3,5-Difluoro-benzyl)-2-hydroxy-3-(7-pyrimidin-5-yl-1,2,3,4-tetrahydro-naphthalen-1-ylamino)-propyl}-acetamide, N-{1-(3,5-Difluoro-benzyl)-2-hydroxy-3-(7-pyridin-2-y-1,2,3,4-tetrahydro-naphthalen-1-ylamino)-propyl}-acetamide, N-{1-(3,5-Difluoro-benzyl)-2-hydroxy-3-[7-(5-methyl-pyridin-2-yl)-1,2,3,4-tetrahydro-naphthalen-1-ylamino]-propyl}-acetamide, N-{1-(3,5-Difluoro-benzyl)-2-hydroxy-3-(7-pyridin-3-yl-1,2,3,4-tetrahydro-naphthalen-1-ylamino)-propyl}-acetamide, N-{1-(3,5-Difluoro-benzyl)-2-hydroxy-3-[7-(3-methyl-pyridin-2-yl)-1,2,3,4-tetrahydro-naphthalen-1-ylamino]-propyl}-acetamide, N-{1-(3,5-Difluoro-benzyl)-2-hydroxy-3-[7-(6-methyl-pyridazin-3-yl)-1,2,3,4-tetrahydro-naphthalen-1-ylamino]-propyl}-acetamide, N-{1-(3,5-Difluoro-benzyl)-2-hydroxy-3-(7-pyridin-4-yl-1,2,3,4-tetrahydro-naphthalen-1-ylamino)-propyl}-acetamide, N-{1-(3,5-Difluoro-benzyl)-2-hydroxy-3-[7-(6-methyl-pyridin-3-yl)-1,2,3,4-tetrahydro-naphthalen-1-ylamino]-propyl}-acetamide, N-{1-(3,5-Difluoro-benzyl)-2-hydroxy-3-[7-(6-methoxy-pyridazin-3-yl)-1,2,3,4-tetrahydro-naphthalen-1-ylamino]-propyl}-acetamide, N-{1-(3,5-Difluoro-benzyl)-2-hydroxy-3-[7-(4-methyl-pyridin-3-yl)-1,2,3,4-tetrahydro-naphthalen-1-ylamino]-propyl}-acetamide, N-{1-(3,5-Difluoro-benzyl)-2-hydroxy-3-(7-pyrazin-2-y-1,2,3,4-tetrahydro-naphthalen-1-ylamino)-propyl}-acetamide, N-{1-(3,5-Difluoro-benzyl)-2-hydroxy-3-[7-(3,6-dimethyl-pyrazin-2-yl)-1,2,3,4-tetrahydro-naphthalen-1-ylamino]-2-hydroxy-propyl}-acetamide, N-{1-(3,5-Difluoro-benzyl)-2-hydroxy-3-[7-(5-methyl-thiophen-2-yl)-1,2,3,4-tetrahydro-naphthalen-1-ylamino]-propyl}-acetamide, N-{1-(3,5-Difluoro-benzyl)-2-hydroxy-3-(7-furan-2-yl-1,2,3,4-tetrahydro-naphthalen-1-ylamino)-2-hydroxy-propyl}-acetamide, N-{1-(3,5-Difluoro-benzyl)-2-hydroxy-3-(7-thiazol-2-yl-1,2,3,4-tetrahydro-naphthalen-1-ylamino)-propyl}-acetamide, N-{1-(3,5-Difluoro-benzyl)-2-hydroxy-3-(7-thiophen-3-yl-1,2,3,4-tetrahydro-naphthalen-1-ylamino)-propyl}-acetamide, N-{1-(3,5-Difluoro-benzyl)-2-hydroxy-3-(7-styryl-1,2,3,4-tetrahydro-naphthalen-1-ylamino)-propyl}-acetamide, N-{1-(3,5-Difluoro-benzyl)-2-hydroxy-3-[7-(3,5-dimethyl-isoxazol-4-yl)-1,2,3,4-tetrahydro-naphthalen-1-ylamino]-2-hydroxy-propyl}-acetamide, N-{1-(3,5-Difluoro-benzyl)-2-hydroxy-3-[7-(1-methyl-1H-imidazol-2-yl)-1,2,3,4-tetrahydro-naphthalen-1-ylamino]-propyl}-acetamide, N-{1-(3,5-Difluoro-benzyl)-2-hydroxy-3-(7-thiophen-2-yl-1,2,3,4-tetrahydro-naphthalen-1-ylamino)-propyl}-acetamide, N-{1-(3,5-Difluoro-benzyl)-2-hydroxy-3-[7-(3-methyl-thiophen-2-yl)-1,2,3,4-tetrahydro-naphthalen-1-ylamino]-propyl}-acetamide, N-{1-(3,5-Difluoro-benzyl)-2-hydroxy-3-[7-(5-ethyl-pyrimidin-2-yl)-1,2,3,4-tetrahydro-naphthalen-1-ylamino]-2-hydroxy-propyl}-acetamide, N-{1-(3,5-Difluoro-benzyl)-2-hydroxy-3-[7-(4-methyl-pyridin-2-yl)-1,2,3,4-tetrahydro-naphthalen-1-ylamino]-propyl}-acetamide, N-{1-(3,5-Difluoro-benzyl)-2-hydroxy-3-(7-isopropenyl-1,2,3,4-tetrahydro-naphthalen-1-ylamino)-propyl}-acetamide, N-[3-{[5-(3-aminophenyl)-7-ethyl-1,2,3,4-tetrahydronaphthalen-1-yl]amino}-1-(3,5-difluorobenzyl)-2-hydroxypropyl]acetamide, N-(1-(3,5-difluorobenzyl)-3-{[7-ethyl-5-(1,3-thiazol-2-yl)-1,2,3,4-tetrahydronaphthalen-1-yl]amino}-2-hydroxypropyl)acetamide, N-(1-(3,5-difluorobenzyl)-3-{[7-ethyl-5-pyridin-2-yl-1,2,3,4-tetrahydronaphthalen-1-yl]amino}-2-hydroxypropyl)acetamide, N-(1-(3,5-difluorobenzyl)-3-{[7-ethyl-5-(3-methylpyridin-2-yl)-1,2,3,4-tetrahydronaphthalen-1-yl]amino}-2-hydroxypropyl)acetamide, N-(1-(3,5-difluorobenzyl)-3-{[7-ethyl-5-(4-methylpyridin-2-yl)-1,2,3,4-tetrahydro naphthalen-1-yl]amino)-2-hydroxypropyl)acetamide, N-(1-[4-(benzyloxy)-3-fluorobenzyl]-3-{[7-(2,2-dimethylpropyl)-1,2,3,4-tetrahydro naphthalen-1-yl]amino}-2-hydroxypropyl)acetamide, N-[3-{[7-(2,2-dimethylpropyl)-1,2,3,4-tetrahydronaphthalen-1-yl]amino}-1-(3-fluoro-4-hydroxybenzyl)-2-hydroxypropyl]acetamide, N-{1-(3,5-Difluoro-benzyl)-3-[6-(2,2-dimethyl-propyl)-1-methyl-1,2,3,4-tetrahydro-quinolin-4-ylamino]-2-hydroxy-propyl}-acetamide, N-{1-(3,5-Difluoro-benzyl)-3-[7-(2,2-dimethyl-propyl)-4-oxo-1,2,3,4-tetrahydro-naphthalen-1-ylamino]-2-hydroxy-propyl}-acetamide, N-(1-(3,5-difluorobenzyl)-3-{[7-(2,2-dimethylpropyl)-5-ethyl-1,2,3,4-tetrahydro naphthalen-1-yl]amino}-2-hydroxypropyl)acetamide, N-(1-(3,5-difluorobenzyl)-3-{[7-(2,2-dimethylpropyl)-1,2,3,4-tetrahydronaphthalen-1-yl]amino}-2-hydroxypropyl)acetamide, N-(1-(3,5-difluorobenzyl)-3-{[6-(2,2-dimethylpropyl)-1,2,3,4-tetrahydroquinolin-4-yl]amino}-2-hydroxypropyl)acetamide, N-(1-(3,5-difluorobenzyl)-3-{[7-(2,2-dimethylpropyl)-1-methyl-1,2,3,4-tetrahydro naphthalen-1-yl]amino}-2-hydroxypropyl)acetamide, N-(1-(3,5-difluorobenzyl)-3-{[7-(2,2-dimethylpropyl)-1,2,3,4-tetrahydro naphthalen-1-yl]amino}-2-hydroxypropyl)-2-fluoroacetamide, N-(1-(3,5-difluorobenzyl)-2-hydroxy-3-{[7-propyl-1,2,3,4-tetrahydronaphthalen-1-yl]amino}propyl)acetamide, N-(1-(3,5-difluorobenzyl)-3-{[7-ethyl-1,2,3,4-tetrahydronaphthalen-1-yl]amino}-2-hydroxypropyl)-2-ethoxyacetamide, N-(1-(3,5-difluorobenzyl)-3-[[7-ethyl-1,2,3,4-tetrahydronaphthalen-1-yl]amino}-2-hydroxypropyl)-2,2-difluoroacetamide, N-[1-(3,5-Difluoro-benzyl)-2-hydroxy-3-(6-isopropyl-2-oxo-1,2,3,4-tetrahydro-quinolin-4-ylamino)-propyl]-acetamide, N-[1-(3,5-Difluoro-benzyl)-2-hydroxy-3-(7-isopropyl-3-oxo-1,2,3,4-tetrahydro-naphthalen-1-ylamino)-propyl]-acetamide, N-[1-(3,5-Difluoro-benzyl)-2-hydroxy-3-(3-hydroxy-7-isopropyl-3-methyl-1,2,3,4-tetrahydro-naphthalen-1-ylamino)-propyl]-acetamide, N-[3-(3-Acetylamino-7-isopropyl-1,2,3,4-tetrahydro-naphthalen-1-ylamino)-1-(3,5-difluoro-benzyl)-2-hydroxy-propyl]-acetamide, N-[1-(3,5-Difluoro-benzyl)-2-hydroxy-3-(7-isopropyl-3-methanesulfonylamino-1,2,3,4-tetrahydro-naphthalen-1-ylamino)-propyl]-acetamide, N-[3-[7-(2,2-Dimethyl-propyl)-1,2,3,4-tetrahydro-naphthalen-1-ylamino]-1-(3-fluoro-4-hydroxy-benzyl)-2-hydroxy-propyl]-acetamide, N-[3-[7-(2,2-Dimethyl-propyl)-1,2,3,4-tetrahydro-naphthalen-1-ylamino]-2-hydroxy-1-(5-hydroxy-pyridin-2-ylmethyl)-propyl]-acetamide, N-{1-(3,5-difluorobenzyl)-2-hydroxy-3-[1,2,3,4-tetrahydronaphthalen-1-ylamino]propyl}acetamide, N-{1-(3,5-difluorobenzyl)-2-hydroxy-3-[(7-methoxy-1,2,3,4-tetrahydronaphthalen-1-yl)amino]propyl}acetamide, N-(1-(3,5-difluorobenzyl)-3-{[7-ethyl-1,2,3,4-tetrahydronaphthalen-1-yl]amino}-2-hydroxypropyl)acetamide, N-(1-(3,5-difluorobenzyl)-3-{[7-ethyl-1,2,3,4-tetrahydronaphthalen-1-yl]amino}-2-hydroxypropyl)acetamide, N-{1-(3,5-difluorobenzyl)-3-[(7-ethyl-1,2,3,4-tetrahydronaphthalen-1-yl)amino]-2-hydroxypropyl)acetamide, N-{1-(3,5-difluorobenzyl)-3-[(6-ethyl-1-methyl-1,2,3,4-tetrahydroquinolin-4-yl)amino]-2-hydroxypropyl}acetamide, N-(1-(3,5-difluorobenzyl)-2-hydroxy-3-{[7-propyl-1,2,3,4-tetrahydronaphthalen-1-yl]amino}propyl)acetamide, N-[1-(3,5-difluorobenzyl)-3-({7-[(dimethylamino)methyl]-1,2,3,4-tetrahydro naphthalen-1-yl)amino)-2-hydroxypropyl]acetamide, and N-[3-{[7-bromo-1,2,3,4-tetrahydronaphthalen-1-yl]amino}-1-(3,5-difluorobenzyl)-2-hydroxypropyl]acetamide.

### EXAMPLE 36

### Additional Representative Compounds

The following formula (I) compounds can be prepared essentially according to the procedures set forth in the above examples and schemes, as well as those known in the art:
N-(1-(3,5-difluorobenzyl)-2-hydroxy-3-{[6-neopentyl-1,2,3,4-tetrahydroquinolin-4-yl]amino}propyl)acetamide, N-(1-(3,5-difluorobenzyl)-2-hydroxy-3-{[6-isobutyl-1,2,3,4-tetrahydroquinolin-4-yl]amino}propyl)acetamide, N-(1-(3,5-difluorobenzyl)-2-hydroxy-3-{[6-isopropyl-1,2,3,4-tetrahydroquinolin-4-yl]amino}propyl)acetamide, N-[3-{[6-tert-butyl-1,2,3,4-tetrahydroquinolin-4-yl]amino}-1-(3,5-difluorobenzyl)-2-hydroxypropyl]acetamide, N-(1-(3,5-difluorobenzyl)-3-{[6-ethyl-1,2,3,4-tetrahydroquinolin-4-yl]amino}-2-hydroxypropyl)acetamide, N-(1-(3,5-difluorobenzyl)-3-{[7-fluoro-6-isopropyl-1,2,3,4-tetrahydroquinolin-4-yl]amino}-2-hydroxypropyl)acetamide, N-[3-{[6-tert-butyl-7-fluoro-1,2,3,4-tetrahydroquinolin-4-yl]amino}-1-(3,5-difluorobenzyl)-2-hydroxypropyl]acetamide, N-(1-(3,5-difluorobenzyl)-3-{[7-fluoro-6-isobutyl-1,2,3,4-tetrahydroquinolin-4-yl]amino}-2-hydroxypropyl)acetamide, N-(1-(3,5-difluorobenzyl)-3-{[7-fluoro-6-neopentyl-1,2,3,4-tetrahydroquinolin-4-yl]amino}-2-hydroxypropyl)acetamide, N-(1-(3,5-difluorobenzyl)-2-hydroxy-3-{[1-methyl-6-neopentyl-1,2,3,4-tetrahydroquinolin-4-yl]amino}propyl)acetamide, N-(1-(3,5-difluorobenzyl)-2-hydroxy-3-{[6-isobutyl-1-methyl-1,2,3,4-tetrahydroquinolin-4-yl]amino}propyl)acetamide, N-(1-(3,5-difluorobenzyl)-2-hydroxy-3-{[6-isopropyl-1-methyl-1,2,3,4-tetrahydroquinolin-4-yl]amino}propyl)acetamide, N-[3-{[6-tert-butyl-1-methyl-1,2,3,4-tetrahydroquinolin-4-yl]amino}-1-(3,5-difluorobenzyl)-2-hydroxypropyl]acetamide, N-[3-{[6-tert-butyl-1-(2-hydroxyethyl)-1,2,3,4-tetrahydroquinolin-4-yl]amino}-1-(3,5-difluorobenzyl)-2-hydroxypropyl]acetamide, N-(1-(3,5-difluorobenzyl)-2-hydroxy-3-{[1-(2-hydroxyethyl)-6-isopropyl-1,2,3,4-tetrahydroquinolin-4-yl]amino}propyl)acetamide, N-(1-(3,5-difluorobenzyl)-2-hydroxy-3-{[1-(2-hydroxyethyl)-6-isobutyl-1,2,3,4-tetrahydroquinolin-4-yl]amino}propyl)acetamide, N-(1-(3,5-difluorobenzyl)-2-hydroxy-3-{[1-(2-hydroxyethyl)-6-neopentyl-1,2,3,4-tetrahydroquinolin-4-yl]amino}propyl)acetamide, N-[3-{[1-acetyl-6-neopentyl-1,2,3,4-tetrahydroquinolin-4-yl]amino}-1-(3,5-difluorobenzyl)-2-hydroxypropyl]acetamide, N-[3-{[1-acetyl-6-isobutyl-1,2,3,4-tetrahydroquinolin-4-yl]amino)-1-(3,5-difluorobenzyl)-2-hydroxypropyl]acetamide, N-[3-{[1-acetyl-6-isopropyl-1,2,3,4-tetrahydroquinolin-4-yl]amino}-1-(3,5-difluorobenzyl)-2-hydroxypropyl]acetamide, N-[3-{[1-acetyl-6-tert-butyl-1,2,3,4-tetrahydroquinolin-4-yl]amino}-1-(3,5-difluorobenzyl)-2-hydroxypropyl]acetamide, N-[3-{[6-tert-butyl-1-(cyanomethyl)-1,2,3,4-tetrahydroquinolin-4-yl]amino}-1-(3,5-difluorobenzyl)-2-hydroxypropyl]acetamide, N-[3-{[1-(cyanomethyl)-6-isopropyl-1,2,3,4-tetrahydroquinolin-4-yl]amino}-1-(3,5-difluorobenzyl)-2-hydroxypropyl]acetamide, N-[3-{[1-(cyanomethyl)-6-isobutyl-1,2,3,4-tetrahydroquinolin-4-yl]amino}-1-(3,5-difluorobenzyl)-2-hydroxypropyl]acetamide, N-[3-{[1-(cyanomethyl)-6-neopentyl-1,2,3,4-tetrahydroquinolin-4-yl]amino}-1-(3,5-difluorobenzyl)-2-hydroxypropyl]acetamide, N-(1-(3,5-difluorobenzyl)-2-hydroxy-3-{[6-(1-hydroxy-2,2-dimethylpropyl)-1,2,3,4-tetrahydroquinolin-4-yl]amino)propyl)acetamide, N-(1-(3,5-difluorobenzyl)-2-hydroxy-3-{[6-(1-hydroxy-2,2-dimethylpropyl)-1-methyl-1,2,3,4-tetrahydroquinolin-4-yl]amino)propyl)acetamide, N-(1-(3,5-difluorobenzyl)-3-{[2,2-dimethyl-6-neopentyl-1,2,3,4-tetrahydroquinolin-4-yl]amino)-2-hydroxypropyl)acetamide, N-(1-(3,5-difluorobenzyl)-2-hydroxy-3-{[1,2,2-trimethyl-6-neopentyl-1,2,3,4-tetrahydroquinolin-4-yl]amino}propyl)acetamide, N-(1-(3,5-difluorobenzyl)-3-{[1,4-dimethyl-6-neopentyl-1,2,3,4-tetrahydroquinolin-4-yl]amino)-2-hydroxypropyl)acetamide, N-(1-(3,5-difluorobenzyl)-2-hydroxy-3-{[4-methyl-6-neopentyl-1,2,3,4-tetrahydroquinolin-4-yl]amino}propyl)acetamide, N-(1-(3,5-difluorobenzyl)-2-hydroxy-3-{[6-isobutyl-4-methyl-1,2,3,4-tetrahydroquinolin-4-yl]amino}propyl)acetamide, N-(1-(3,5-difluorobenzyl)-2-hydroxy-3-{[6-isobutyl-1,4-dimethyl-1,2,3,4-tetrahydroquinolin-4-yl]amino)propyl)acetamide, N-[3-[(6-tert-butoxy-1,2,3,4-tetrahydroquinolin-4-yl)amino]-1-(3,5-difluorobenzyl)-2-hydroxypropyl]acetamide, N-[3-[(6-tert-butoxy-4-methyl-1,2,3,4-tetrahydroquinolin-4-yl)amino]-1-(3,5-difluorobenzyl)-2-hydroxypropyl]acetamide, N-[3-[(6-tert-butoxy-4,8-dimethyl-1,2,3,4-tetrahydroquinolin-4-yl)amino]-1-(3,5-difluorobenzyl)-2-hydroxypropyl]acetamide, N-{1-(3,5-difluorobenzyl)-2-hydroxy-3-[(4-methyl-6-neopentyl-1,2,3,4-tetrahydroquinolin-4-yl)amino]propyl)acetamide, N-(1-(3,5-difluorobenzyl)-3-[(4,8-dimethyl-6-neopentyl-1,2,3,4-tetrahydroquinolin-4-yl)amino]-2-hydroxypropyl}acetamide, N-{1-(3,5-difluorobenzyl)-2-hydroxy-3-[(8-methyl-6-neopentyl-1,2,3,4-tetrahydroquinolin-4-yl)amino]propyl}acetamide, N-(1-(3,5-difluorobenzyl)-2-hydroxy-3-{[6-(2-hydroxy-2-methylpropyl)-8-methyl-1,2,3,4-tetrahydroquinolin-4-yl]amino}propyl)acetamide, N-(1-(3,5-difluorobenzyl)-2-hydroxy-3-{[6-(2-hydroxy-2-methylpropyl)-4-methyl-1,2,3,4-tetrahydroquinolin-4-yl]amino}propyl)acetamide, N-(1-(3,5-difluorobenzyl)-2-hydroxy-3-{[6-(2-hydroxy-2-methylpropyl)-1,2,3,4-tetrahydroquinolin-4-yl]amino}propyl)acetamide, and N-(1-(3,5-difluorobenzyl)-2-hydroxy-3-{[6-(1-hydroxy-2,2-dimethylpropyl)-4-methyl-1,2,3,4-tetrahydroquinolin-4-yl]amino}propyl)acetamide.

### EXAMPLES 37-45

### PREPARATION OF ISOTHIOCHROMAN 2,2-DIOXIDE INTERMEDIATES AND FORMULA (I) COMPOUNDS

### EXAMPLE 37

### GENERAL PROCEDURE FOR PREPARING EXAMPLE COMPOUNDS

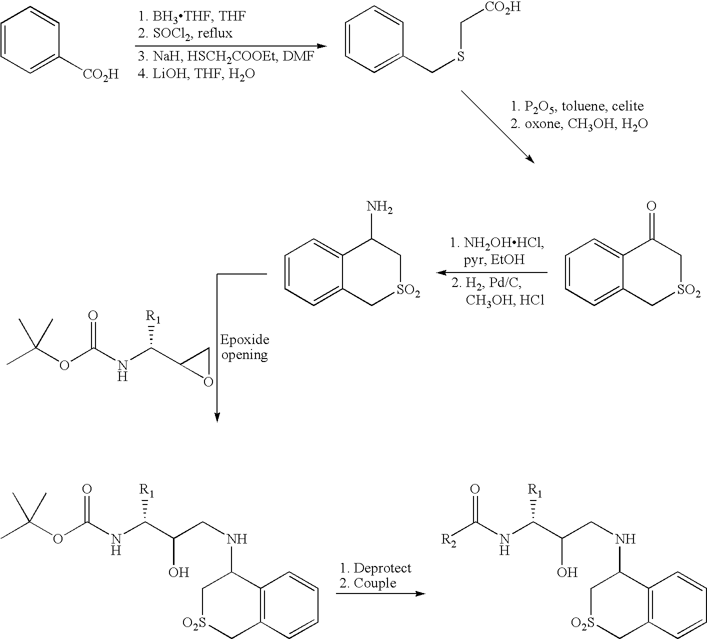 where R₁ and R₂ are defined above.

The above scheme illustrates the preparation of compounds wherein R_{C} is an isothiochroman 2,2-dioxide using an optionally substituted benzoic acid as the starting material. One of skill in the art will recognize that optionally substituted benzyl halides or benzyl alcohols may also be used as starting materials.

In the first reaction sequence in the above scheme, benzoic acid is reduced to benzyl alcohol, which is then converted into benzyl halide. Alternatively, benzyl alcohol may be modified to include a leaving group such as, for example, tosylate, brosylate, nosylate, triflate or mesylate. The benzyl compound is then reacted with a sulfide to generate the thioether which is then hydrolyzed to form a carboxylic acid. In the second reaction sequence, this acid is subjected to annulation reaction conditions to form the desired bicyclic ring system. The annulation can be carried out using a Lewis acid, polyphosphoric acid, or P₂O₅. Other suitable reagents that effect cyclization are known in the art.

The resulting bicyclic sulfide is oxidized to form the sulfone. The keto group is converted into an amine directly via reductive amination or indirectly through the generation of an oxime, which is then reduced to form the amine. Transition metal catalysts and hydrogen or other reducing agents, such as NaBH₄, LiAlH₄ or NaCNBH₃, may be used to effect the reduction.

The resulting amine is used to open the epoxide to form the resulting coupled product. The coupled product is then deprotected to form a free amine, which is acylated or sulfonylated to generate the desired final product. In the above scheme, the use of a Boc protecting group is illustrated, but one of skill in the art will appreciate that other protecting groups, such as CBz, benzyl or others can also be used.

### EXAMPLE 38

### Alternative Procedure for Preparing Example Compounds

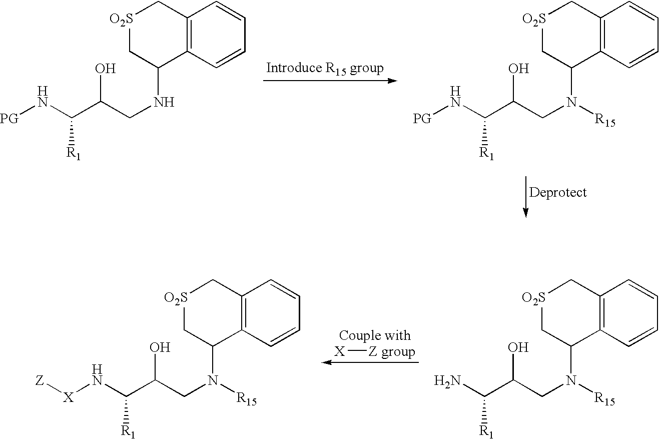

The above scheme illustrates the introduction of a non-hydrogen R₁₅ group on the 3-position nitrogen atom in the 1,3-diaminopropane portion of the molecule. The free nitrogen is reacted with an electrophile, an aldehyde or ketone and a reducing agent, an acid chloride, an acid anhydride or an acid with a coupling agent, such as DCC (dicyclohexyl carbodiimide), DIC (1,3 diisopropyl carbodiimide), EDCl (1-ethyl-3-(3′-dimethylaminopropyl)carbodiimide hydrochloride), BBC (1-benzotriazol-1-yloxy-bis(pyrrolidino)uronium hexafluorophosphate), BDMP (5-(1H-benzotriazol-1-yloxy)-3,4-dihydro-1-methyl 2H-pyrrolium hexachloroanitimonate), BOMI (benzotriazol-1-yloxy-N,N-dimethylmethaniminium hexachloroantimonate), HATU (O(7-aza benzotriazol-1-yl)-1,1,3,3-tetramethyluronium hexafluorophosphate), HAPyU =O-(7-azabenzotriazol-1-yl)-1,1,3,3-bis(tetramethylene)uronium hexafluorophosphate, HBTU which is O-(benzotriazol-1-yl)-1,1,3,3-tetramethyluronium hexafluoro phosphate, TAPipU which is O-(7-azabenzotriazol-1-yl)-1,1,3,3-bis (pentamethylene)uronium tetrafluoroborate, AOP (O-(7-azabenzotriazol-1-yl)-tris(dimethylamino)phosphonium hexafluorophosphate), BDP (benzotriazol-1-yl diethyl phosphate), BOP (1-benzotriazolyoxytris(dimethylamino) phosphonium hexafluorophosphate), PyAOP (7-azobenzotriazolyoxytris(pyrrolidino) phosphonium hexafluorophosphate), PyBOP (1-benzotriazolyoxytris(pyrrolidino) phosphonium hexafluorophosphate), TDBTU (2-(3,4-dihydro-4-oxo-1,2,3-benzotriazin-3-yl)-1,1,3,3-tetramethyluronium tetrafluoroborate), TNTU (2-(5-norbornene-2,3-dicarboximido)-1,1,3,3-tetramethyluronium tetrafluoroborate), TPTU (2-(2-oxo-1(2H)-pyridyl-1,1,3,3-tetramethyluronium tetrafluoroborate), TSTU (2-succinimido-1,1,3,3-tetramethyl uronium tetrafluoroborate), BEMT (2-bromo-3-ethyl-4-methyl thiazolium tetrafluoroborate), BOP-Cl (bis(2-oxo-3-oxazolidinyl)phosphinic chloride), BroP (bromotris(dimethylamino)phosphonium hexafluorophosphate), BTFFH (bis(tetramethylenefluoroformamidinium) hexafluorophosphate), CIP (2-chloro-1,3-dimethylimidazolidinium hexafluorophosphate), DEPBT (3-(diethoxyphosphoryloxy)-1,2,3-benzotriazin-4(3M-one), Dpp-Cl (diphenylphosphinic chloride), EEDQ (2-ethoxy-1-ethoxycarbonyl-1,2-dihydroquinoline), FDPP (pentafluorophenyl diphenyl phosphinate), HOTT (S(1-oxido-2-pyridinyl)-1,1,3,3-tetramethylthiouronium hexafluorophosphate), PyBroP (bromotris(pyrrolydino)phophonium hexafluoro phosphate), PyCloP (chlorotris(pyrrolydino)phophonium hexafluorophosphate), TFFH (tetramethylfluoroformamidinium hexafluorophosphate), and TOTT (S(1-oxido-2-pyridinyl)-1,1,3,3-tetramethylthiouronium tetrafluoroborate) to generate the monosubstituted product, which can then be deprotected and coupled to the “X-Z” group. Conversely, the monosubstituted product can be deprotected, and the free nitrogen reacted with an electrophile, an aldehyde or ketone and a reducing agent, an acid chloride, an acid anhydride or an acid with a coupling agent, such as those previously exemplified to generate the disubstituted product, which is then coupled to the “X-Z” group.

### EXAMPLE 39

### Alternative Procedure for Preparing Example Compounds

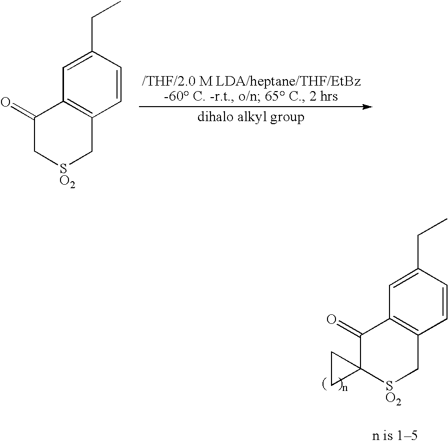

Spirocycles can be synthesized by alkylating a compound in the presence of a strong base. Examples of strong bases include LDA, KHMDS, and tertiary-butyl lithium. One of skill in the art will appreciate that many other bases are strong enough to deprotonate the starting material and effect the desired transformation.

The alkylating agent dictates the size of the spirocycle that is formed. Dibromo ethane, diiodoethane, or bromo iodoethane will yield a spirocyclopropyl compound, wherein n is 1. However, longer alkyl chains yield larger spirocycloalkyl compounds. For example, a 1,5-dihalopentane generates a spirocyclohexyl compound, wherein n is 4. Although dihalo compounds are illustrated, one of skill in the art will appreciate that other leaving groups, such as, for example, mesylate, tosylate, triflate, brosylate, and nosylate may be used. The leaving groups may, but need not be, identical.

### EXAMPLE 40

### Preparation of Representative Chroman Intermediates

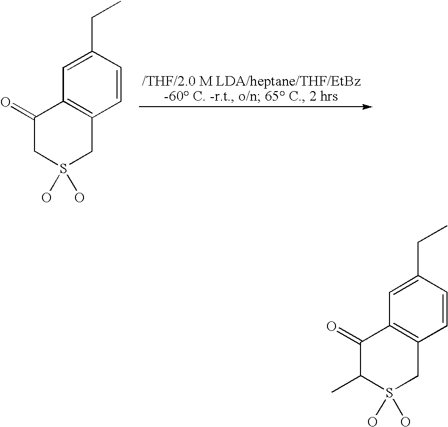

Lithium diisopropylamine (LDA) in heptane/THF/ethylbenzene (LDA (2.5 mL of a 2M heptane/THF/ethylbenzene solution, 5 mmol, 1.25 eq.) was added to the sulfone ketone (0.9 g, 4 mmol) in 40 mL of THF at −60° C. The mixture was stirred for about 15 min, and then methyl iodide (1.24 mL, 20 mmol, 5 eq.) was added. The reaction mixture was stirred for 1 h at −60° C., then the cold bath was removed, and then the reaction was stirred overnight. The reaction was then partitioned between EtOAc and water, washed with 0.5N HCl, aqueous sodium bicarbonate solution, and brine, dried (sodium sulfate), filtered and concentrated. The concentrate was purified by column chromatography to yield 0.68 g of the desired product as an oil, which solidified upon standing. TLC (30% EtOAc/Hexane, Rf=0.39). MS m/z 239.1.

### EXAMPLE 41

### Substituted Ureas and Carbamates Ureas an Carbamates

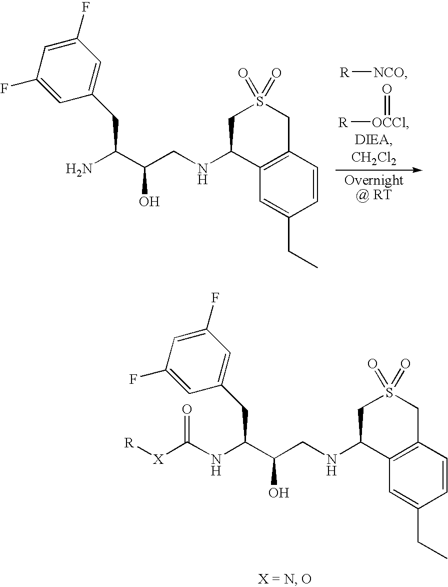

The reaction was run in 4 mL vials. The starting amine (0.07 mmol) was placed in each reaction vial and diisopropylethylamine (0.28 mmol, 4 eq) was added. Either isocyanate or chloroformate (0.077 mmol, 1.1 eq) was then added. Finally, the starting reagents were dissolved in dichloromethane (1.5 mL). Each reaction was run overnight at room temperature. LC/MS analysis for each reaction was performed via an Agilent 1100 HPLC, utilizing a Thermo-Hypersil C18 50×3 mm 5 micron column, coupled to a Thermo-Finnigan LCQ MS. Final purification of each product was performed via a Varian Pro Star Preparative HPLC utilizing a Phenomenex C18 60×21.2 mm 5 micron column.

### EXAMPLE 42

### PREPARATION OF N-((1S,2R)-1-[3-(ALLYLOXY)-5-FLUOROBENZYL]-3-{[(4R)-6-ETHYL-2,2-DIOXIDO-3,4-DIHYDRO-1H-ISOTHIOCHROMAN-4-YL]AMINO}-2-HYDROXYPROPYL)ACETAMIDE

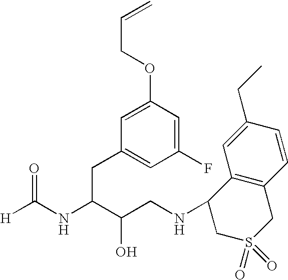

Using methods analogous to those previously described, tert-butyl (1S)-2-[3-(allyloxy)-5-fluorophenyl-1-[(2S)-oxiran-2-yl]ethylcarbamate (0.37 mmol) and (4R)-6-ethyl-3,4-dihydro-1H-isothiochroman-4-amine 2,2-dioxide (0.78 mmol) were reacted together, and the product was further converted using methods analogous to those previously described, (except that the HCl salt is not formed) to N-((1S,2R)-1-[3-(allyloxy)-5-fluorobenzyl]-3-{[(4R)-6-ethyl-2,2-dioxido-3,4-dihydro-1H-isothiochroman-4-yl]amino}-2-hydroxypropyl)acetamide (0.16 mmol, 43%), which was obtained as a white solid: ¹H NMR (CDCl₃) δ 7.22-7.19 (m, 2 H), 7.13 (m, 1 H), 6.57 (m, 1 H), 6.51 (m, 2 H), 6.06-5.99 (m, 1 H), 5.75 (br, 1 H), 5.41 (d, J=17 Hz, 1 H), 5.30 (d, J=12 Hz, 1 H), 4.67 (d, J=15 Hz, 1 H), 4.50 (m, 2 H), 4.26 (m, 1 H), 4.17 (d, J=15 Hz, 1 H), 4.1 (m, 1 H), 3.66 (m, 2 H), 3.48 (m, 1 H), 3.36 (dd, 1 H), 2.90 (m, 2 H), 2.78 (m, 2 H), 2.67 (q, J=7.6 Hz, 2 H), 1.91 (s, 3 H), 1.25 (t, J=7.6 Hz, 3 H); MS (CI) m/z 505.4 [M+H]⁺.

### EXAMPLE 43

### PREPARATION OF N-((1S,2R)-1-(CYCLOHEXYLMETHYL)-3-{[(4R)-6-ETHYL-2,2-DIOXIDO-3,4-DIHYDRO-1H-ISOTHIOCHROMEN-4-YL]AMINO}-2-HYDROXYPROPYL)ACETAMIDE

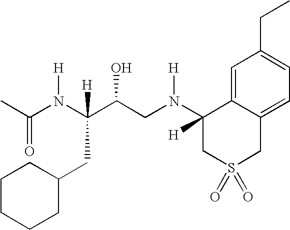

Using methods analogous to those previously described, tert-butyl (1S)-2-cyclohexyl-1-[(2S)-oxiran-2-yl]ethylcarbamate (0.91 mmol) and (4R)-6-ethyl-3,4-dihydro-1H-isothiochromen-4-amine 2,2-dioxide (1.15 mmol) were combined. The resulting product was recovered by chromatography over silica gel, eluting with 3% methanol (containing 1% NH₄OH) in CH₂Cl₂. This material was then converted to N-((1S,2R)-1-(cyclohexylmethyl)-3-{[(4R)-6-ethyl-2,2-dioxido-3,4-dihydro-1H-isothiochromen-4-yl]amino)-2-hydroxypropyl)acetamide, which was obtained as a white solid: MS (CI) m/z 437.3 [M+H]⁺.

### EXAMPLE 44

### PREPARATION OF (1S,2R)-1-(CYCLOHEXYLMETHYL)-3-{[(4R)-6-ETHYL-2,2-DIOXIDO-3,4-DIHYDRO-1H-ISOTHIOCHROMEN-4-YL]AMINO}-2-HYDROXYPROPYLFORMAMIDE

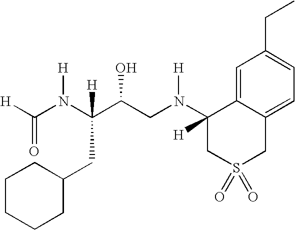

Using methods analogous to those previously described, tert-butyl (1S)-2-cyclohexyl-1-[(2S)-oxiran-2-yl]ethylcarbamate (0.91 mmol) and (4R)-6-ethyl-3,4-dihydro-1H-isothiochromen-4-amine 2,2-dioxide (1.15 mmol) were combined. The resulting product (0.63 mmol, 69%) was purified by chromatography over silica gel, eluting with 3% methanol (containing 1% NH₄OH) in CH₂Cl₂. The purified material was then converted to (1S,2R)-1-(cyclohexylmethyl)-3-{[(4R)-6-ethyl-2,2-dioxido-3,4-dihydro-1H-isothiochromen-4-yl]amino}-2-hydroxypropylformamide (obtained as a white solid), using methods analogous to those disclosed herein. MS (CI) m/z 423.3 [M+H]⁺.

### EXAMPLE 45

### Example Compounds

The following compounds are prepared essentially according to the procedures set forth in the above examples and schemes:
N-{1-(3,5-Difluoro-benzyl)-3-[6-(2,2-dimethyl-propyl)-thiochromen-4-ylamino]-2-hydroxy-propyl}-acetamide, N-{1-(3,5-Difluoro-benzyl)-3-[6-(2,2-dimethyl-propyl)-1-oxo-1λ⁴-thiochromen-4-ylamino]-2-hydroxypropyl}-acetamide, N-{1-(3,5-Difluoro-benzyl)-3-[6-(2,2-dimethyl-propyl)-1,1-dioxo-1λ⁶-thiochromen-4-ylamino]-2-hydroxy-propyl}-acetamide, N-(1-(3,5-difluorobenzyl)-2-hydroxy-3-{[6-isopropyl-2,2-dioxido-3,4-dihydro-1H-isothiochromen-4-yl]amino}propyl)acetamide, N-(1-(3,5-difluorobenzyl)-2-hydroxy-3-{[6-isopropyl-2,2-dioxido-3,4-dihydro-1H-isothiochromen-4-yl]amino}propyl)acetamide, N-{1-(3,5-difluorobenzyl)-3-[(6-ethyl-2,2-dioxido-3,4-dihydro-1H-isothiochromen-4-yl)amino]-2-hydroxypropyl}acetamide, N-{1-(3,5-difluorobenzyl)-3-[(6-ethyl-2,2-dioxido-3,4-dihydro-1H-isothiochromen-4-yl)amino]-2-hydroxypropyl}acetamide, N-{1-(3,5-difluorobenzyl)-3-[(2,2-dioxido-3,4-dihydro-1H-isothiochromen-4-yl)amino]-2-hydroxypropyl}acetamide, N-[1-(3,5-Difluoro-benzyl)-3-(6-ethyl-2,2-dioxo-2λ⁶-isothiochromen-4-ylamino)-2-hydroxy-propyl]-2-methylamino-acetamide, 2-amino-N-[1-(3,5-difluoro-benzyl)-3-(6-ethyl-2,2-dioxo-2λ⁶-isothiochromen-4-ylamino)-2-hydroxy-propyl]-acetamide, N-{1-(3,5-difluorobenzyl)-3-[(6-ethyl-3,4-dihydro-1H-isothiochromen-4-yl)amino]-2-hydroxypropyl}acetamide, N-[l-(3,5-Difluoro-benzyl)-3-(6-ethyl-2,2-dioxo-2λ⁶-isothiochromen-4-ylamino)-2-hydroxy-propyl]-2-phenyl-acetamide, N-{1-(3,5-difluorobenzyl)-3-[(6-ethyl-3-methyl-2,2-dioxido-3,4-dihydro-1H-isothiochromen-4-yl)amino]-2-hydroxypropyl}acetamide, N-{1-(3,5-difluorobenzyl)-3-[(6-ethyl-3-methyl-2,2-dioxido-3,4-dihydro-1H-isothiochromen-4-yl)amino]-2-hydroxypropyl}acetamide, N-[1-(3,5-Difluoro-benzyl)-3-(6-ethyl-2,2-dioxo-2λ⁶-isothiochromen-4-ylamino)-2-hydroxy-propyl]-2-(1H-imidazol-4-yl)-acetamide, and
N-(1-(cyclohexylmethyl)-3-{[6-ethyl-2,2-dioxido-3,4-dihydro-1H-isothiochromen-4-yl]amino}-2-hydroxypropyl)acetamide.

### EXAMPLE 46

### General Procedure for Preparing Representative Compounds

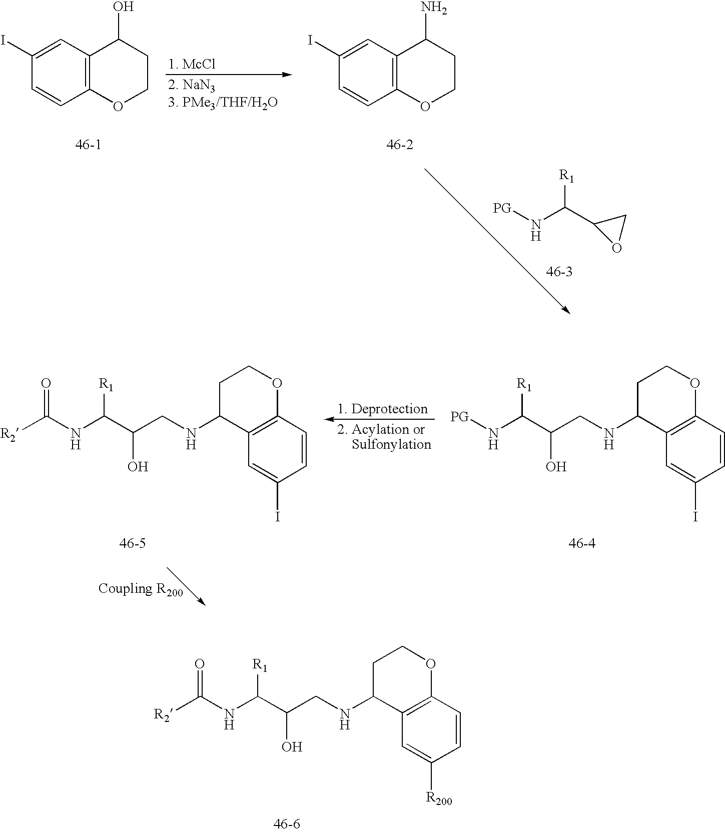

As described above and below, one embodiment of the invention provides for compounds of formula 46-6 as shown above. These compounds may be made by methods known to those skilled in the art from starting compounds that are also known to those skilled in the art. The process chemistry is further well known to those skilled in the art. A suitable process for the preparation of compounds of formula 46-6 is set forth in the above scheme, which illustrates the preparation of the desired compounds using the readily obtainable 6-iodo-chroman-4-ol as a starting material (see Synthesis, 1997, 23-25). One skilled in the art will recognize that there are several methods for the conversion of the alcohol functionality to the desired amino compounds of formula 46-2. The alcohol 46-1 is first activated with methane sulfonyl chloride and the resulting mesylate displaced with sodium azide NaN₃. Alternative methods for the conversion of an alcohol to an azide are well known to one skilled in the art. The resulting azide is subsequently reduced using trimethylphosphine in a mixture of THF and water. One skilled in the art will recognize that there are several methods for the reduction of an azide to the corresponding amine. For examples, see Larock, R. C. in Comprehensive Organic Transformations, Wiley-VCH Publishers, 1999. This reduction of the azide produces a mixture of enantiomers of the amine 46-2. This enantiomeric mixture can be separated by means known to those skilled in the art such as low temperature recrystallization of a chiral salt or by chiral preparative HPLC, most preferably by HPLC, employing commercially available chiral columns.

The resulting amine 46-2 is used to open the epoxide 46-3 to yield the protected (6-iodo-3,4-dihydro-2H-chromen-4-yl)amino propyl carbamate 46-4. Suitable reaction conditions for opening the epoxide 46-3 include running the reaction in a wide range of common and inert solvents. C₁-C₆ alcohol solvents are preferred and isopropyl alcohol most preferred. The reactions can be run at temperatures ranging from 20-25° C. up to the reflux temperature of the alcohol employed. The preferred temperature range for conducting the reaction is between 50° C. and the refluxing temperature of the alcohol employed.

The protected iodo-chromen 46-4 is deprotected to the corresponding amine by means known to those skilled in the art for removal of amine protecting groups. Suitable means for removal of the amine protecting group depend on the nature of the protecting group. Those skilled in the art, knowing the nature of a specific protecting group, know which reagent is preferable for its removal. For example, it is preferred to remove the preferred protecting group, Boc, by dissolving the protected iodo-chroman in a trifluoroacetic acid/ dichloromethane (1/1) mixture. When complete the solvents are removed under reduced pressure to give the corresponding amine (as the corresponding salt, i.e. trifluoroacetic acid salt) which is used without further purification. Alternatively, the amine can be purified further by means well known to those skilled in the art, such as for example recrystallization. Further, if the non-salt form is desired, it can be obtained by means known to those skilled in the art, such as for example, preparing the free base amine via treatment of the salt with mild basic conditions. For additional deprotection conditions and deprotection conditions for other protecting groups, see, for example, T. W. Green and P. G. M. Wuts in Protecting Groups in Organic Chemistry, 3^{rd} edition, John ‘Wiley and Sons, 1999.

After deprotection, the amine is reacted with an appropriately substituted amide forming agent, Z-(CO)—Y, to produce coupled amides 46-5 by nitrogen acylation means known to those skilled in the art. Nitrogen acylation conditions for the reaction of amine with an amide forming agent Z-(CO)—Y are known to those skilled in the art and can be found in R. C. Larock in Comprehensive Organic Transformations, VCH Publishers, 1989, p. 981, 979, and 972. Y can be —OH (carboxylic acid) or halide (acyl halide), preferably chlorine, imidazole (acyl imidazole), or a suitable group to produce a-mixed anhydride.

The acylated iodo-chromen 46-5 is coupled with an appropriately functionalized organometallic R₂₀₀M to yield compounds of formula 46-6 using conditions known to those skilled in the art. One skilled in the art will recognize that there are several methods for coupling various alkyl and aryl groups to an aromatic iodide. For examples, see L. S. Hegedus Transition Metals in the Synthesis of Complex Organic Molecules, University Science, 1999.

### EXAMPLE 47

### General Procedure for Preparing Representative Compounds

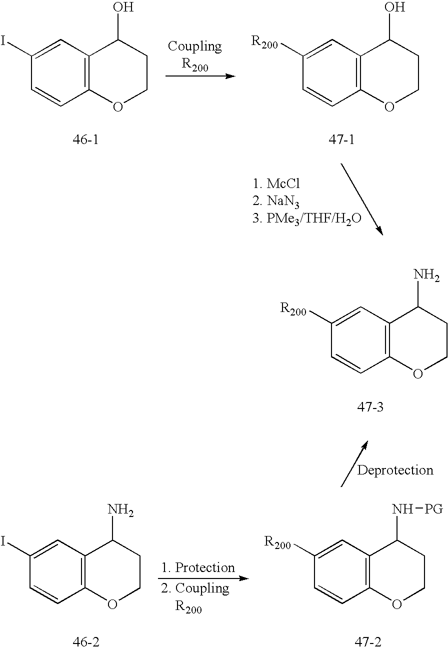

The above scheme sets forth alternative synthetic routes to 4-aminochromanes, which are useful for preparing compounds of formula 46-6. Amines of formula 47-3 can be prepared by coupling the appropriately functionalized organometallic to 6-iodo-chroman-4-ol 46-1 or to the appropriately protected iodo-amino chroman of the formula 46-2. Further elaboration of the coupled products using methods known to one of skill in the art, ultimately yields the desired amines of formula 47-3. The chemistry from this point forward follows the generalizations described in Example 48 for converting compound 47-3 to 46-6.

### EXAMPLE 48

### Preparation of Bicyclic Amines (Isochromen Compounds)

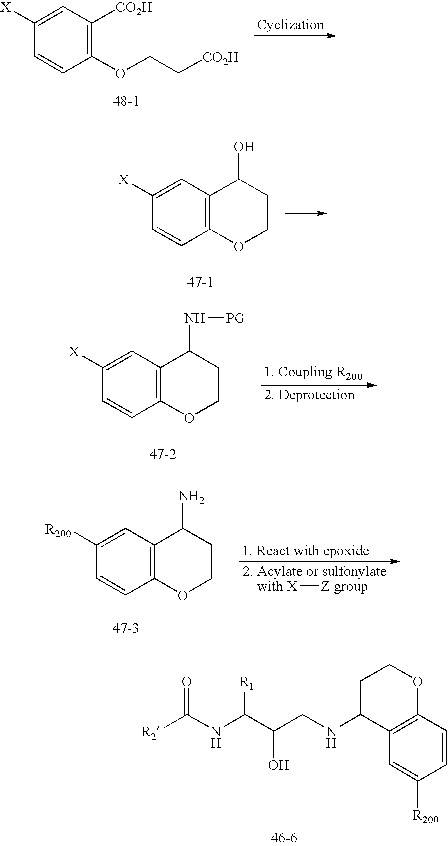

The above scheme illustrates another general preparation of amines of formula 47-3 that upon following the generalizations outlined in the above schemes will result in compounds of the formula 46-6. For the following examples, the chemistry is essentially the same as described for the schemes in Examples 71-77.

### EXAMPLE 49

### Example Compounds

The following compounds of formula (I) are prepared essentially according to the procedures described in the schemes and preparations set forth above:
N-{(1S,2R)-1-(3,5-difluorobenzyl)-2-hydroxy-3-[(6-isopropyl-3,4-dihydro-2H-chromen-4-yl)amino]propyl}acetamide, N-((1S,2R)-1-(3,5-difluorobenzyl)-2-hydroxy-3-{[(4S)-6-isobutyl-3,4-dihydro-2H-chromen-4-yl]amino}propyl)acetamide, N-{(1S,2R)-1-(3,5-difluorobenzyl)-2-hydroxy-3-[(6-neopentyl-3,4-dihydro-2H-chromen-4-yl)amino]propyl}acetamide, N-[(1S ,2 R)-3-{[(4S)-6-cyano-3,4-dihydro-2H-chromen-4-yl]amino}-1-(3,5-difluorobenzyl)-2-hydroxypropyl]acetamide, and N-((1S,2R)-1-(3,5-difluorobenzyl)-2-hydroxy-3-{[(4S)-6-(1H-pyrrol-3-yl)-3,4-dihydro-2H-chromen-4-yl]amino}propyl)acetamide.

### EXAMPLE 50

### PREPARATION OF N-[(1S,2R)-1-(3,5-DIFLUOROBENZYL)-3-(3,4-DIHYDRO-2H-CHROMEN-4-YLAMINO)-2-HYDROXYPROPYL]ACETAMIDE

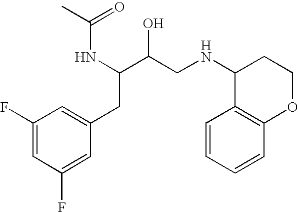

### Step 1: Chromen-4-ol

NaBH₄ (5.5 g, 145 mmol) was added to a MeOH (250 mL) solution of 4-chromenone (16.6 g, 11 mmol), at 0° C., in 1 g portions over a 30 min period. After complete addition the mixture was stirred for 1 h and allowed to warm to room temperature. The reaction was quenched with the slow addition of aq. NH₄Cl (100 mL). The MeOH was removed in vacuo and the residue extracted with Et₂O (2×100 mL). The organic layers were dried (magnesium sulfate) and treated with activated carbon. After filtration the Et₂O was removed in vacuo to yield 15.8 g of chromen-4-ol as a clear oil. HRMS (ESI+) calc'd for C₉H₁₀O₂ m/z 150.0681 [M+H]⁺; found 150.0679.

### Step 2: 3,4-dihydro-2H-chromen-4-ylamine

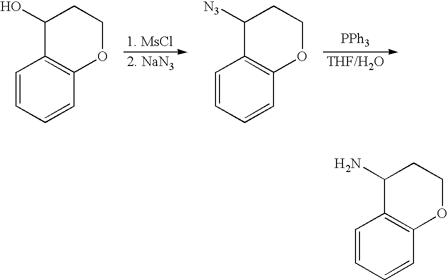

MsCl (2.1 mL, 27 mmol) was added to a CH₂Cl₂ (80 mL) solution of chromen-4-ol (3.1 g, 20.6 mmol) and DIEA (8 mL, 42 mmol) at 0° C. via syringe. After complete addition, the cold bath was removed and stirring continued at room temperature. After 15 h, the CH₂Cl₂ was removed in vacuo and the residue was dissolved in 80 mL of DMF, which was followed by the addition of NaN₃ (1.8 g, 27 mmol). The-mixture was heated to 75° C. (oil bath) for 5 h then cooled to room temperature. The mixture was diluted with Et₂O (400 mL) and washed with 1 N HCl, NaHCO₃, and brine. The organic layer was dried (sodium sulfate) and concentrated in vacuo to yield the azide as a yellow oil. ¹H NMR (400 MHz, CDCl₃) δ 7.27-7.21 (m, 2 H), 6.97-6.87 (m, 2 H), 4.61 (appt, J=3.84 Hz, 1 H), 4.31-4.19 (m, 2 H), 2.18 (m, 1 H), 2.03 (m, 1 H). MS (ESI−) for C₉H₁₀N₃O m/z 173.0 [M−H]⁻.

The crude azide was dissolved in 60 mL of THF followed by the addition of PPh₃ (6.5 g, 25 mmol) and the mixture was stirred at room temperature for 30 min. The mixture was treated with 8 mL of H₂O and heated to 60° C. (oil bath) overnight. The mixture was concentrated in vacuo and the resulting residue treated with 1 N HCl. The aqueous mixture was extracted with CH₂Cl₂ and then the pH was adjusted to 12 with NaOH. The mixture was then re-extracted with CH₂Cl₂. The second CH₂Cl₂ layers were combined, dried (sodium sulfate), and concentrated in vacuo to yield 3,4-dihydro-2H-chromen-4-ylamine as a slightly yellow oil. HRMS (ESI+) calc'd for C₉H₁₁NO m/z 150.0919 [M+H]⁺; found 150.0920.

### Step 3: tert-butyl (1S,2R)-1-(3,5-difluorobenzyl)-3-(3,4-dihydro-2H-chromen-4-ylamino)-2-hydroxypropylcarbamate

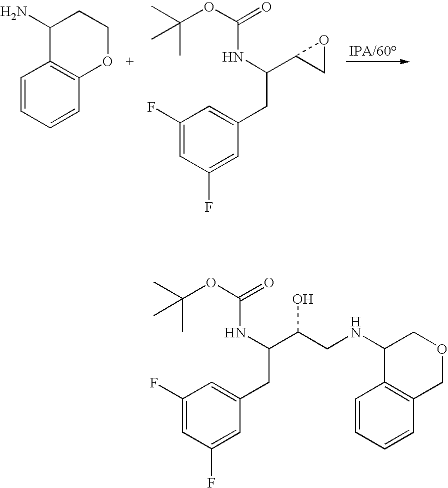

A solution of tert-butyl (1S)-2-(3,5-difluorophenyl)-1-[(2S)-oxiran-2-yl]ethylcarbamate (0.54 g, 1.8 mmol) and 3,4-dihydro-2H-chromen-4-ylamine (0.40 g, 2.6 mmol) in IPA (15 mL) was heated at 60° C. (oil bath) with stirring overnight. The IPA was removed in vacuo and the residue dissolved in EtOAc and washed with 1 N HCl. The organic layer was dried (magnesium sulfate) and concentrated in vacuo to yield 0.75 g of the desired product as a mixture of epimers. HRMS (ESI+) calc'd for C₂₄H₃₀N₂O₄F₂ m/z 449.2252 [M+H]⁺; found 449.2258.

### Step 4: N-[(1S,2R)-1-(3,5-difluorobenzyl)-3-(3,4-dihydro-2H-chromen-4-ylamino)-2-hydroxypropyl]acetamide

N-[(1S,2R)-1-(3,5-difluorobenzyl)-3-(3,4-dihydro-2H-chromen-4-ylamino)-2-hydroxypropyl]acetamide, which was obtained as a clear glass, was prepared essentially according to the procedure described in Example 3, steps 7-8. Preparative reverse phase HPLC yields two fractions:

¹H NMR (400 MHz, CDCl₃) δ 7.29 (m, 1 H), 7.20 (m, 1 H), 6.92 (m, 1 H), 6.85 (dd, J=6.85, 0.93 Hz, 1 H), 6.79-6.67 (m, 3 H), 5.69 (d, J=8.91 Hz, 1 H), 4.35-4.23 (m, 2 H), 4.15 (m, 1 H), 3.87 (m, 1 H), 3.58 (m, 1 H), 3.03 (m, 1 H), 2.91-2.75 (m, 3 H), 2.15-2.08 (m, 1 H), 2.04-1.99 (m, 1H), 1.94 (s, 3 H). MS (ESI+) for C₂₁H₂₄F₂N₂O₃ m/z 391.3 [M+H]⁺.

¹H NMR (400 MHz, CDCl₃) δ 7.31 (m, 1 H), 7.21 (m, 1 H), 6.93 (m, 1 H), 6.86 (dd, J=8.29, 1.04 Hz, 1 H), 6.79-6.67 (m, 3 H), 5.69 (d, J=8.91 Hz, 1 H), 4.36-4.24 (m, 2 H), 4.17 (m, 1 H), 3.87 (appt, J=4.04 Hz, 1 H), 3.54 (m, 1 H), 3.03 (dd, J=14.31, 4.56 Hz, 1 H), 2.95 (m, 1 H), 2.88-2.79 (m, 2 H), 2.16-2.00 (m, 2 H), 1.92 (s, 3 H). MS (ESI+) for C₂₁H₂₄F₂N₂O₃ m/z 391.3 [M+H]⁺.

### EXAMPLE 51

### PREPARATION OF N-((1S,2R)-1-(3,5-DIFLUOROBENZYL)-3-{[(4S)-6-ETHYL-3,4-DIHYDRO-2H-CHROMEN-4-YL]AMINO}-2-HYDROXYPROPYL)ACETAMIDE

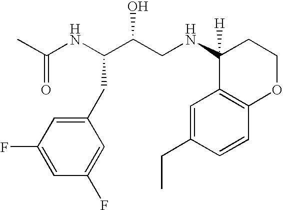

### Step 1: 6-iodochroman-4-ol

HgO (29.7 g, 137 mmol) and 12 (34.8 g, 137 mmol) were added to a solution of chroman-4-ol (19.6 g, 131 mmol) in CH₂Cl₂ (500 mL), at room temperature, under N₂(g). After stirring for 48 h, the mixture was filtered through a plug of silica gel and the plug washed with 30% EtOAc/hexanes. The filtrate was washed with 15% Na₂S₂O₃ and the organic layer was dried over Na₂CO₃, filtered, and concentrated in vacuo, yielding 6-iodochroman-4-ol as an off-white solid (32.44 g, 90% crude yield). Recrystallization was performed by dissolving the product in hot dichloromethane (250 mL) and slowly adding petroleum ether (250 mL). Overall yield 25.9 g, 72% yield. Anal. Calc'd for C₉H₉IO₂; C, 39.16; H, 3.29; found C, 39.26; H, 3.27.

### Step 2: 6-Iodo-chroman-4-ylamine

MsCl (4.2 mL, 54 mmol) was added to a solution of 6-iodo-4-chromanol (10.0 g, 36 mmol) and diisopropylethyl amine (19 mL, 108 mmol), in CH₂Cl₂ (80 mL) at 0° C. After stirring for 1.5 h, the solvent was removed in vacuo and the resulting residue dissolved in 150 mL of DMF followed by the addition of NaN₃ (3.5 g, 54 mmol). The reaction was heated to 70° C. for 6.5 h then cooled to room temperature followed by the addition of 900 mL of 1 N HCl and extraction with Et₂O. The combined Et₂O layers were dried (magnesium sulfate) and concentrated in vacuo to yield 9.5 g of the azide as yellow oil. MS (ESI+) for C₉H₈IN₃O m/z 300.97 [M+H]⁺.

The crude azide (5.0 g, 16.6 mmol) was dissolved in THF (50 mL) and treated with PPh₃ (5.2 g, 20.0 mmol). The mixture stirred at room temperature for 30 min followed by the addition of 4 mL of H₂O. The mixture was then heated to 60° C. overnight. After cooling the mixture was concentrated in vacuo and the resulting residue treated with 1 N HCl. The aqueous layer was washed with CH₂Cl₂ and then adjusted to pH=12 with NaOH pellets. The basic aqueous layer was extracted with CH₂Cl₂ and the combined organic layers dried (sodium sulfate) and treated with activated carbon. The mixture was filtered through Celite® and concentrated in vacuo to yield 6-Iodo-chroman-4-ylamine 3.6 g (79%) as a clear oil that solidifies upon standing. HRMS (ESI+) calc'd for C₉H₁₀INO m/z 275.9887 [M+H]⁺ found 275.9893.

### Step 3: tert-butyl (1S,2R)-1-(3,5-difluorobenzyl)-2-hydroxy-3-[(6-iodo-3,4-dihydro-2H-chromen-4-yl)amino]propylcarbamate

The above compound was prepared essentially according to the procedure described in Example 50, step 3; it was obtained as a mixture of diastereomers, which was used without purification. MS (ESI+) for C₂₄H₂₉F₂IN₂O₄ m/z 574.8 [M+H]⁺.

### Step 4: N-{(1S,2R)-1-(3,5-difluorobenzyl)-2-hydroxy-3-[(6-iodo-3,4-dihydro-2H-chromen-4-yl)amino]propyl}acetamide

The title compound was obtained from the propylcarbamate, essentially according to the methods described herein, as a light yellow solid. MS (ESI+) for C₂₁H₂₃F₂IN₂O₃ m/z 517.0 [M+H]⁺. Chiral preparative HPLC (20% IPA/Heptane, 0.1% DEA) yields the two diastereomers.

N-((1S ,2R)-1-(3,5-difluorobenzyl)-2-hydroxy-3-{[(4S)-6-iodo-3,4-dihydro-2H-chromen-4-yl]amino}propyl)acetamide ¹H NMR (400 MHz, DMSO-d₆) δ 7.73 (d, J=9.12 Hz, 1 H), 7.62 (d, J=2.07 Hz, 1 H), 7.40 (dd, J=8.50, 2.28 Hz, 1 H), 7.01 (m, 1 H), 6.89 (m, 2 H), 6.58 (d, J=8.50 Hz, 1 H), 4.97 (d, J=6.01 Hz, 1 H), 4.23 (m, 1 H), 4.14 (m, 1 H), 3.93.(m, 1 H), 3.68 (m, 1 H), 3.47 (m, 1 H), 3.01 (dd, J=13.89, 3.32 Hz, 1 H), 2.61 (m, 2 H), 1.90 (m, 2 H), 1.71 (s, 3 H).

N-((1S,2R)-1-(3,5-difluorobenzyl)-2-hydroxy-3-{[(4R)-6-iodo-3,4-dihydro-2H-chromen-4-yl]amino}propyl)acetamide ¹H NMR (400 MHz, DMSO-d₆) δ 7.75 (d, J=9.33 Hz, 1 H), 7.64 (d, J=2.07 Hz, 1 H), 7.41 (dd, J=8.60, 2.18 Hz, 1 H), 7.02 (m, 1 H), 6.92 (m, 2 H), 6.59 (d, J=8.50 Hz, 1 H), 4.96 (d, J=5.80 Hz, 1 H), 4.22 (m, 1 H), 4.15 (m, 1 H), 3.95 (m, 1 H), 3.68 (m, 1 H), 3.45 (m, 1 H), 2.98 (dd, J=13.99, 2.80 Hz, 1 H), 2.73 (m, 1 H), 2.63-2.57 (m, 1 H), 1.87 (m, 2 H), 1.70 (s, 3 H).

### Step 5: N-((1S,2R)-1-(3,5-difluorobenzyl)-3-{[6-ethyl-3,4-dihydro-2H-chromen-4-yl]amino}-2-hydroxypropyl)acetamide

N-{(1S,2R)-1-(3,5-difluorobenzyl)-2-hydroxy-3-[(6-iodo-3,4-dihydro-2H-chromen-4-yl)amino]propyl}acetamide (1.0 g, 1.9 mmol) and Pd(dppf)Cl₂ (0.078 g, 0.1 mmol) were dissolved in 20 mL of degassed THF. 10 mL of 2.0 M K₃PO₄ was added to the mixture followed by the addition of Et₃B (3.8 mL, 3.8 mmol, 1.0 M in THF) via syringe. The reaction mixture was heated to 65° C. under N₂(g). After 2.5 h, the reaction was complete. It was then diluted with EtOAc (100 mL) and washed with brine, dried (sodium sulfate) and concentrated in vacuo to yield brown solid. The diastereomers of N-((1S,2R)-1-(3,5-difluorobenzyl)-3-{[(4S)-6-ethyl-3,4-dihydro-2H-chromen-4-yl]amino}-2-hydroxypropyl)acetamide were separated by preparative chiral HPLC (Chiralpak AD, 20% IPA/80% heptane, 0.1% DEA). MS (ESI+) for C₂₃H₂₆F₂N₂O₃ m/z 419 [M+H]⁺.

CH₂Cl₂ (5 mL), MeOH (0.5 mL), and N-((1S,2R)-1-(3,5-difluorobenzyl)-3-{[(4S)-6-ethyl-3,4-dihydro-2H-chromen-4-yl]amino}-2-hydroxypropyl)acetamide (0.2 g, 0.5 mmol), and 1N HCl in Et₂O (0.38 mL) were added to a solution of MTBE (20 mL). The mixture was stirred at room temperature. The final white solid was isolated by removing the solvent and trituration with Et₂O. HRMS (ESI+) calc'd for C₂₃H₂₈F₂N₂O₃ m/z 419.2146 [M+H]⁺; found 419.2166.

### EXAMPLE 52

### PREPARATION OF N-((1S,2R)-1-(3,5-DIFLUOROBENZYL)-2-HYDROXY-3-{[(4S)-6-ISOBUTYL-3,4-DIHYDRO-2H-CHROMEN-4-YL]AMINO}PROPYL)ACETAMIDE

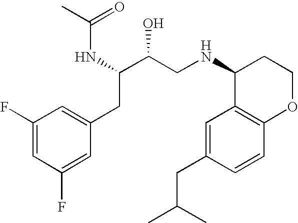

### Step 1: (4R)-6-iodochroman-4-ol

The above compound was prepared essentially according to the procedure described in Example 51, step 1. Chiral HPLC separation was performed at this stage. HRMS (EI) calc'd for C₉H₉IO₂ 275.9649, found 275.9646. (4S)-6-iodochromen-4-ol [α]²⁰_{D}=+13 (20 mg, MeOH); (4R)-6-iodochromen-4-ol [α]²⁰_{D}=−13 (20 mg, MeOH).

### Step 2: (4S)-6-iodochroman-4-amine

Diphenylphosphoryl azide (6.42 mL, 29.76 mmol) was added to a solution of (4R)-6-iodochromen-4-ol (6.85 g, 24.81 mmol) in toluene (100 mL) under N₂(g) at 0° C. A chilled solution of DBU (4.45 mL, 29.76 mmol) in toluene was added via syringe. The reaction mixture was allowed to warm to room temperature overnight. The azide solution was filtered through silica gel using 6:1 hexanes:EtOAc as the eluant. The filtrate was concentrated in vacuo, then dissolved in anhydrous THF (100 mL), then 1.0M Me₃P in THF (29.76 mL, 29.76 mmol) was added. After 1 h, deionized H₂O (5 mL) was added and reaction mixture was stirred overnight under N₂(g). The mixture was concentrated in vacuo, dissolved in EtOAc, and washed with 10% NaHCO₃ and brine. The organic layers were then dried (sodium sulfate), filtered, and concentrated in vacuo to give (4S)-6-iodochroman-4-amine as a white solid. ¹H NMR (400 MHz, CDCl₃) δ 1.70 (s, 2 H), 1.86 (m, 1 H), 2.13 (m, 1 H), 4.03 (t, J=5 Hz, 1 H), 4.23 (m, 2 H), 6.60 (d, J=9 Hz, 1 H), 7.42 (d, J=9 Hz, 1 H), 7.64 (s, 1 H). MS (ESI+) for C₉H₁₀INO m/z 258.8 [M+H]⁺.

### Step 3: tert-Butyl (1S,2R)-1-(3,5-difluorobenzyl)-2-hydroxy-3-{[(4S)-6-iodo-3,4-dihydro-2H-chromen-4-yl]amino}propylcarbamate.

The above compound was prepared essentially according to the method of Example 50, step 3. The crude product was purified via column chromatography using 3% MeOH/DCM as eluant. The desired compound was obtained as a colorless solid (6.89 g, 79%). HRMS (ESI); calc'd for C₂₄H₂₉N₂O₄IF₂+H¹ 575.1220, found 575.1194; Specific Rotation (25 C D)=30 (c=1.04) MeOH.

### Step 4: N-((1S,2R)-1-(3,5-difluorobenzyl)-2-hydroxy-3-{[(4S)-6-iodo-3,4-dihydro-2H-chromen-4-yl]amino}propyl)acetamide.

The title compound was prepared using procedures described herein, and isolated as a yellow solid. ¹H NMR (400 MHz, CDCl₃) δ 1.93 (s, 3 H), 1.97 (m, 1 H), 2.08 (m, 1 H), 2.80 (m, 3 H), 3.09 (dd, J=4, 14 Hz, 1 H), 3.55 (m, 1 H), 3.84 (m, 1 H), 4.13 (m, 1 H), 4.24 (m, 1 H), 4.31 (m, 1 H), 5.61 (m, 1 H), 6.62 (d, J=9 Hz, 1 H), 6.70 (m, 1 H), 6.77 (d, J=6 Hz, 2 H), 7.44 (dd, J=2, 9 Hz, 1 H), 7.62 (s, 1 H).

### Step 5: N-((1S,2R)-1-(3,5-difluorobenzyl)-2-hydroxy-3-{[(4S)-6-isobutyl-3,4-dihydro-2H-chromen-4-yl]amino}propyl)acetamide.

Pd(dppf)Cl₂ (0.024 g, 0.03 mmol) was added to a solution of the product from step 4 (0.300 g, 0.58 mmol) in anhydrous THF (2.3 mL), and then stirred under N₂(g). Isobutylzinc bromide (9.2 mL of a 0.5M THF solution, 4.6 mmol) was added to this solution and the reaction mixture was stirred overnight. The reaction was quenched with methanol and then Dowex 50WX2-400 resin (used in excess, 4.6 meq/g) was added. The mixture was filtered through a frit and the resin was washed with methanol. The alkylated material was released from the resin using 7N NH₃/MeOH. The filtrate was concentrated in vacuo and then purified via preparative HPLC to yield a colorless solid fully characterized as the HCl salt.

3 equiv of HCl (in MeOH) were added to N-((1S,2R)-1-(3,5-difluorobenzyl)-2-hydroxy-3-{[(4S)-6-isobutyl-3,4-dihydro-2H-chromen-4-yl]amino}propyl)acetamide (2.0 g, 4.5 mmol) in MeOH. (10 mL), at 0° C. The reaction yielded N-((1S,2R)-1-(3,5-difluorobenzyl)-2-hydroxy-3-{[(4S)-6-isobutyl-3,4-dihydro-2H-chromen-4-yl]amino}propyl)acetamide hydrochloride (1.97 g) as a white powder, after trituration with CH₂Cl₂. HRMS (ESI+) calc'd for C₂₅H₃₂F₂N₂O₃ m/z 447.2459 [M+H]⁺; found 447.2440. Anal calc'd for C₂₅H₃₂F₂N₂O₃.HCl; C, 62.17; H, 6.89; N, 5.80; found C, 62.68; H, 7.05; N, 5.75.

### EXAMPLE 53

### Example Compounds

The following compounds of formula (I) are prepared essentially according to the procedures described in the schemes and preparations set forth above:
N-[3-{[6-(2-cyanophenyl)-3,4-dihydro-2H-chromen-4-yl]amino}-1-(3,5-difluorobenzyl)-2-hydroxypropyl]acetamide, N-[3-{[6-(4-cyanophenyl)-3,4-dihydro-2H-chromen-4-yl]amino}-1-(3,5-difluorobenzyl)-2-hydroxypropyl]acetamide, N-[3-{[6-sec-butyl-3,4-dihydro-2H-chromen-4-yl]amino}-1-(3,5-difluorobenzyl)-2-hydroxypropyl]acetamide, N-[3-{[6-cyclopentyl-3,4-dihydro-2H-chromen-4-yl]amino}-1-(3,5-difluorobenzyl)-2-hydroxypropyl]acetamide, N-(1-(3,5-difluorobenzyl)-3-{[6-(1,1-dimethylpropyl)-3,4-dihydro-2H-chromen-4-yl]amino}-2-hydroxypropyl)acetamide, N-[3-{[6-cyclohexyl-3,4-dihydro-2H-chromen-4-yl]amino}-1-(3,5-difluorobenzyl)-2-hydroxypropyl]acetamide, N-(1-(3,5-difluorobenzyl)-2-hydroxy-3-{[6-(3-methylbutyl)-3,4-dihydro-2H-chromen-4-yl]amino}propyl)acetamide, N-[3-{[6-(2-cyanobenzyl)-3,4-dihydro-2H-chromen-4-yl]amino}-1-(3,5-difluorobenzyl)-2-hydroxypropyl]acetamide, N-[3-{[6-(4-cyanobenzyl)-3,4-dihydro-2H-chromen-4-yl]amino)-1-(3,5-difluorobenzyl)-2-hydroxypropyl]acetamide, N-[3-({6-[bicyclo[2.2.1]hept-2-yl]-3,4-dihydro-2H-chromen-4-yl}amino)-1-(3,5-difluorobenzyl)-2-hydroxypropyl]acetamide, N-(1-(3,5-difluorobenzyl)-2-hydroxy-3-{[6-(1-methylbutyl)-3,4-dihydro-2H-chromen-4-yl]amino}propyl)acetamide, N-(1-(3,5-difluorobenzyl)-2-hydroxy-3-{[6-(1-methylpentyl)-3,4-dihydro-2H-chromen-4-yl]amino)propyl)acetamide, N-(1-(3,5-difluorobenzyl)-3-{[6-(1-ethylpropyl)-3,4-dihydro-2H-chromen-4-yl]amino}-2-hydroxypropyl)acetamide, N-(1-(3,5-difluorobenzyl)-3-{[6-(1-ethylbutyl)-3,4-dihydro-2H-chromen-4-yl]amino}-2-hydroxypropyl)acetamide, N-(1-(3,5-difluorobenzyl)-2-hydroxy-3-{[6-(1-propylbutyl)-3,4-dihydro-2H-chromen-4-yl]amino}propyl)acetamide, N-(1-(3,5-difluorobenzyl)-3-{[6-(2-ethylbutyl)-3,4-dihydro-2H-chromen-4-yl]amino}-2-hydroxypropyl)acetamide, N-[3-{[6-(cyclohexylmethyl)-3,4-dihydro-2H-chromen-4-yl]amino}-1-(3,5-difluorobenzyl)-2-hydroxypropyl]acetamide, N-[3-{[6-(5-cyano-5-methylhexyl)-3,4-dihydro-2H-chromen-4-yl]amino}-1-(3,5-difluorobenzyl)-2-hydroxypropyl]acetamide, N-(1-(3,5-difluorobenzyl)-2-hydroxy-3-{[6-(4-methoxyphenyl)-3,4-dihydro-2H-chromen-4-yl]amino}propyl)acetamide, N-(1-(3,5-difluorobenzyl)-2-hydroxy-3-[6-(6-methylpyridin-2-yl)-3,4-dihydro-2H-chromen-4-yl]amino}propyl)acetamide, N-(1-(3,5-difluorobenzyl)-2-hydroxy-3-{[6-(5-methylpyridin-2-yl)-3,4-dihydro-2 H-chromen-4-yl]amino)propyl)acetamide, N-(1-(3,5-difluorobenzyl)-2-hydroxy-3-{[6-(4-methylpyridin-2-yl)-3,4-dihydro-2H-chromen-4-yl]amino)propyl)acetamide, N-[3-{[6-(4-cyanobutyl)-3,4-dihydro-2H-chromen-4-yl]amino}-1-(3,5-difluorobenzyl)-2-hydroxypropyl]acetamide, N-[3-{[6-(6-cyanohexyl)-3,4-dihydro-2H-chromen-4-yl]amino}-1-(3,5-difluorobenzyl)-2-hydroxypropyl]acetamide, N-[3-{[6-(3-cyanophenyl)-3,4-dihydro-2H-chromen-4-yl]amino}-1-(3,5-difluorobenzyl)-2-hydroxypropyl]acetamide, 3-(4-{[3-(acetylamino)-4-(3,5-difluorophenyl)-2-hydroxybutyl]amino}-3,4-dihydro-2H-chromen-6-yl)-2-Methylpropanoate, N-(1-(3,5-difluorobenzyl)-3-{[6-(4-fluorophenyl)-3,4-dihydro-2H-chromen-4-yl]amino}-2-hydroxypropyl)acetamide, methyl-3-(4-{[3-(acetylamino)-4-(3,5-difluorophenyl)-2-hydroxybutyl]amino}-3,4-dihydro-2H-chromen-6-yl)-2-Methylpropanoate, N-[1-(3,5-difluorobenzyl)-3-({6-[2-(1,3-dioxolan-2-yl)ethyl]-3,4-dihydro-2H-chromen-4-yl}amino)-2-hydroxypropyl]acetamide, N-(1-(3,5-difluorobenzyl)-2-hydroxy-3-{[6-(6-methoxypyridin-2-yl)-3,4-dihydro-2H-chromen-4-yl]amino}propyl)acetamide, and N-[3-{[6-cyano-3,4-dihydro-2H-chromen-4-yl]amino}-1-(3,5-difluorobenzyl)-2-hydroxypropyl]acetamide.

### EXAMPLE 54

### PREPARATION OF N-((1S,2R)-1-(3,5-DIFLUOROBENZYL)-2-HYDROXY-3-{[(4S)-6-(1 H-PYRROL-3-YL)-3,4-DIHYDRO-2H-CHROMEN-4-YL]AMINO}PROPYL)ACETAMIDE

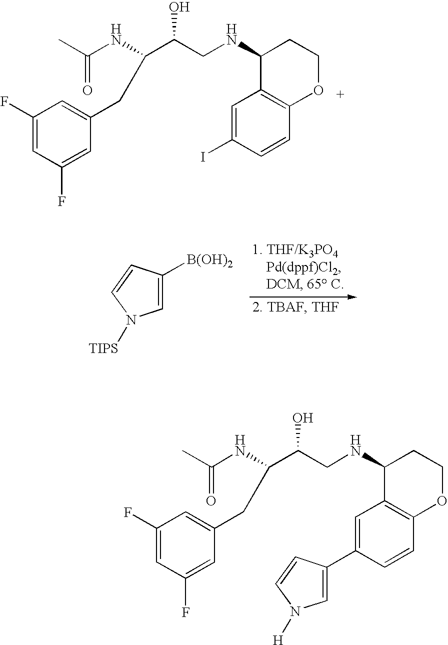

Pd(dppf)Cl₂ (0.030 g, 0.03 mmol) and K₃PO₄ (2.9 mL, 5.80 mmol) were added to a solution of N-((1S,2R)-1-(3,5-difluorobenzyl)-2-hydroxy-3-{[(4S)-6-iodo-3,4-dihydro-2H-chromen-4-yl]amino}propyl)acetamide (0.300 g, 0.58 mmol) in anhydrous THF (5 mL). Boronic acid (0.310 g, 1.16 mmol) (J. Org. Chem., 1992, 57, 1653) was added and the reaction was stirred at 65° C. overnight under N₂(g). The reaction was quenched with deionized water and then extracted with ethyl acetate. The organic layers were washed with brine, dried (magnesium sulfate), filtered, and concentrated in vacuo. The TIPS-protected compound (0.100 g, 0.16 mmol) was dissolved in THF (3 mL) and then a 0.1 M solution of TBAF in THF (0.32 mL, 0.32 mmol) was added. The reaction was stirred for 2 h, concentrated in vacuo, dissolved in ethyl acetate, filtered through a silica gel plug, and concentrated in vacuo to give the desired product, which was an amber oil (130 mg), which was purified by reverse phase prep-HPLC. HRMS (ESI); calc'd for C₂₅H₂₇N₃O₃F₂+H1 456.2099, found 456.2092.

### EXAMPLE 55

### PREPARATION OF N-((1S,2R)-1-(3,5-DIFLUOROBENZYL)-2-HYDROXY-3-{[(4S)-6-NEOPENTYL-3,4-DIHYDRO-2H-CHROMEN-4-YL]AMINO}PROPYL)ACETAMIDE

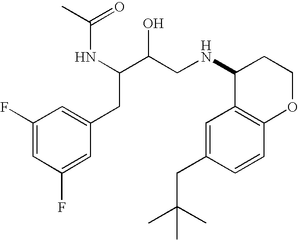

### Step 1: 6-neopentylchroman-4-ol

The Pd(dppf)Cl₂.CH₂Cl₂ (0.15 g, 0.18 mmol), followed by neopentylmagnesium bromide (10.8 mL, 10.8 mmol, 1.0 M in Et₂O) was added to a solution of 6-iodochroman-4-ol (1.0 g, 3.6 mmol) in 18 mL of THF at 0° C. The cold bath was maintained for 10 min, then removed. The reaction was stirred overnight, then quenched with NH₄Cl (30 mL) and extracted with EtOAc. The combined organic layers were dried (magnesium sulfate) and concentrated in vacuo to yield a brown oil. The crude oil was absorbed onto silica gel followed by flash chromatography (biotage 40S) 10% EtOAc/heptanes to yield 0.36 g (46%) of 6-neopentylchromen-4-ol as a white solid. R_{f}=0.11. HRMS (ESI+) calc'd for C₁₄H₂₀O₂ m/z 220.1463 [M+H]⁺; found 220.1460.

### Step 2: 6-neopentyl-3,4-dihydro-2H-chromen-4-ylamine

6-neopentyl-3,4-dihydro-2H-chromen-4-ylamine was prepared essentially according to the procedure of Example 52, Step 2. First, the azide was prepared. ¹H NMR (400 MHz, CDCl₃) δ 6.94 (dd, J=8.40, 2.18 Hz, 1 H), 6.89 (d, J=2.07 Hz, 1 H), 6.71 (d, J=8.29 Hz, 1 H), 4.50 (appt, J=3.73 Hz, 1 H), 4.15 (m, 2 H), 2.36 (s, 2 H), 2.08 (m, 1 H), 1.93 (m, 1 H), 0.83 (s, 9 H). Second, the azide was reduced to yield the amine as a slightly colored oil (1.6 g). The amine was taken to the next step without further purification. HRMS (ESI+) calc'd for C₁₄H₂₁NO m/z 219.1623 [M+H]⁺; found 219.1628.

### Step 3: tert-Butyl (1S,2R)-1-(3,5-difluorobenzyl)-2-hydroxy-3-[(6-neopentyl-3,4-dihydro-2H-chromen-4-yl)amino]propylcarbamate

tert-Butyl (1S,2R)-1-(3,5-difluorobenzyl)-2-hydroxy-3-[(6-neopentyl-3,4-dihydro-2H-chromen-4-yl)amino]propylcarbamate was prepared essentially according to the procedure of Example 50, step 3; it was obtained as an off white solid. Flash chromatography (3% MeOH/CHCl₃, 1 mL of NH₄OH per liter) yielded the desired product as a mixture of epimers. HRMS (ESI+) calc'd for C₂₉H₄₀N₂O₄F₂ m/z 519.3034 [M+H]⁺; found 519.3040.

### Step 4: N-((1S,2R)-1-(3,5-difluorobenzyl)-2-hydroxy-3-{[(4S)-6-neopentyl-3,4-dihydro-2H-chromen-4-yl]amino}propyl)acetamide

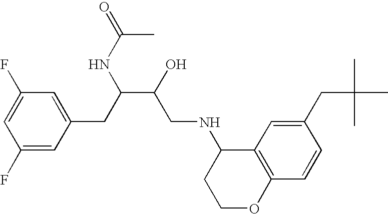

N-((1S,2R)-1-(3,5-difluorobenzyl)-2-hydroxy-3-{[(4S)-6-neopentyl-3,4-dihydro-2 H-chromen-4-yl]amino}propyl)acetamide was prepared essentially according the method of Example 3, steps 7-8, which resulted in a mixture of epimers. The epimers were then separated using chiral preparative HPLC (10% IPA/heptanes, 0.1% DEA) AD column:
N-((1S,2R)-1-(3,5-difluorobenzyl)-2-hydroxy-3-{[(4S)-6-neopentyl-3,4-dihydro-2H- chromen-4-yl]amino}propyl)acetamide. ¹H NMR (400 MHz, CDCl₃) δ 7.01 (d, J=1.87 Hz, 1 H), 6.96 (dd, J=8.29, 2.07 Hz, 1 H), 6.79-6.67 (m, 4 H), 5.69 (d, J=8.50 Hz, 1 H), 4.32-4.15 (m, 3 H), 3.85 (bs, 1 H), 3.60 (bs, 1 H), 3.02 (m, 1 H), 2.88 (m, 2 H), 2.76 (dd, J=12.13, 6.74 Hz, 1 H), 2.46 (s, 2 H), 2.15-2.08 (m, 1 H), 2.04-1.98 (m, 1 H), 1.94 (s, 3 H), 0.91 (s, 9 H). HRMS (ESI+) calc'd for C₂₆H₃₄F₂N₂O₃ m/z 461.2615 [M+H]⁺; found 461.2621.

N-((1S,2R)-1-(3,5-difluorobenzyl)-2-hydroxy-3-{[(4R)-6-neopentyl-3,4-dihydro-2H-chromen-4-yl]amino}propyl)acetamide. ¹H NMR (400 MHz, CDCl₃) δ 7.04 (d, J=2.07 Hz, 1 H), 6.96 (dd, J=8.29, 1.87 Hz, 1 H), 6.77-6.67 (m, 4 H), 5.69 (d, J=8.91 Hz, 1 H), 4.31-4.16 (m, 3 H), 3.86 (bs, 1 H), 3.57 (bs, 1 H), 3.00 (m, 2 H), 2.82 (m, 2 H), 2.44 (s, 2 H), 2.18-2.00 (m, 3 H), 1.90 (s, 3 H), 0.91 (s, 9 H). HRMS (ESI+) calc'd for C₂₆H₃₄F₂N₂O₃ m/z 461.2615 [M+H]⁺; found 461.2630. Anal. Calc'd for C₂₆H₃₄F₂N₂O₃; C, 67.81; H, 7.44; N, 6.08; found C, 67.65; H, 7.51; N, 6.05.

### EXAMPLE 56

### Chiral Synthesis of Amine

### Step 1: (4R)-6-neopentylchromen-4-ol

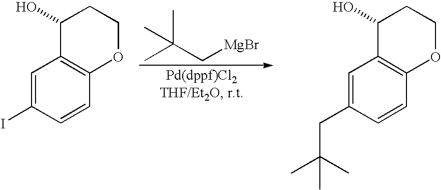

(4R)-6-Iodochroman-4-ol was converted into (4R)-6-neopentylchroman-4-ol essentially according to the procedure of Example 55, step 1. The product was obtained as a white solid. Anal. Calc'd for C₁₄H₂₀O₂; C, 76.33; H, 9.15; found C, 76.31; H, 9.06. [α]_{D}=22.3, c=1.14 (CH₂Cl₂).

### Step 2: (4S)-6-neopentyl-3,4-dihydro-2H-chromen-4-ylamine

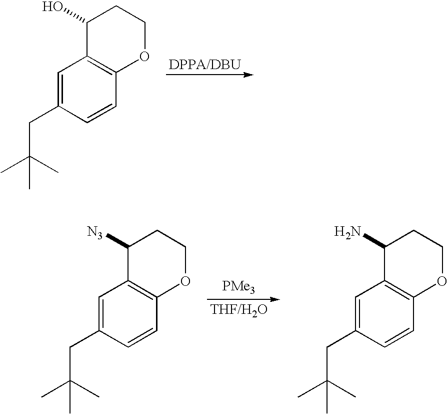

(4R)-6-neopentylchroman-4-ol was converted to (4S)-6-neopentyl-3,4-dihydro-2H-chromen-4-ylamine essentially according to the procedure of Example 52, step 2.

### EXAMPLE 57

### Alternative Chiral Synthesis of Amine

### Step 1

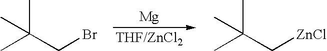

Neopentyl zinc was prepared according to the procedure described in Tetrahedron Lett., 1983, 24, 3823-3824.

### Step 2. tert-butyl (4S)-6-iodo-3,4-dihydro-2H-chromen-4-ylcarbamate

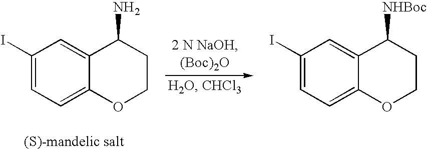

2N sodium hydroxide (21 mL, 42 mmol), followed by di-tert-butyl dicarbonate (2.58 g, 11.7 mmol) and chloroform (50 mL), was added to a suspension of amine (S)-mandelic salt (4.55 g, 10.6 mmol) in water (50 mL). The reaction mixture was stirred at room temperature for 2 h and then diluted with methylene chloride (100 mL) and water (50 mL). The organic layer was separated, washed with saturated sodium chloride, dried (sodium sulfate), filtered, and concentrated under reduced pressure. The residue was triturated with 1:1 hexanes/ethyl ether. The resulting white solid was collected by filtration and washed with hexanes to provided tert-butyl (4S)-6-iodo-3,4-dihydro-2H-chromen-4-ylcarbamate (3.30 g, 83%): ¹H NMR (300 MHz, CDCl₃) δ 7.55 (d, J=1.8 Hz, 1H), 7.42 (dd, J=8.6,2.2 Hz, 1H), 6.58 (d, J=8.6 Hz, 1H), 4.78 (m, 2H), 4.28-4.20 (m, 1H), 4.18-4.10 (m, 1H), 2.19-2.10 (m, 1H), 2.061.96 (m, 1H), 1.49 (s, 9H).

### Step 3: Coupling of neopentyl zinc reagent to tert-butyl (4S)-6-iodo-3,4-dihydro-2H-chromen-4-ylcarbamate

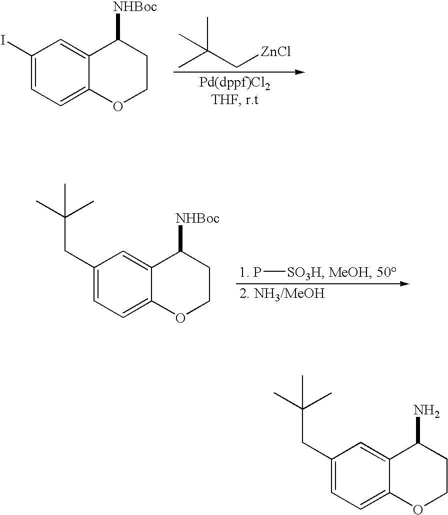

The tert-butyl (4S)-6-iodo-3,4-dihydro-2H-chromen-4-ylcarbamate (1.8 g, 5.0 mmol) and Pd(dppf)Cl₂ (0.2 g, 0.25 mmol) were added to a suspension of the 0.3 M neopentyl zinc reagent in THF (60 mL, 15 mmol) as solids in one portion. The mixture was stirred at room temperature under N₂(g) for 48 h (progress monitored by LC/MS and HPLC). The mixture was quenched with aqueous NH₄Cl and extracted with EtOAc. The organic layer was dried (sodium sulfate) and concentrated in vacuo. The crude residue was dissolved in MeOH (25 mL) and treated with DOWEX® 50WX2-400 ion exchange resin. The mixture was heated to 50° C. for 6 h. The resin was collected by filtration, washed with MeOH and CH₂Cl₂, and treated with 7 N NH₃/MeOH to elute the free amine from the resin. The elutions were concentrated in vacuo to yield a light brown oil (0.63 g, 57%) of (4S)-6-neopentyl-3,4-dihydro-2H-chromen-4-ylamine. 6-neopentyl-3,4-dihydro-2H-chromen-4-ylamine was characterized as the mono.HCl salt. ¹H NMR (300 MHz, DMSO-d₆) δ); 7.25 (s, 1H), 7.02 (m, 1H,), 6.76 (m, 1H), 4.47 (bs, 1H), 4.21 (m, 2H), 2.38 (s, 2H), 2.24 (m, 1H), 2.10 (m, 1H), 0.87 (s, 9H). HRMS (ESI+) calculated for C₁₄H₂₁N₁O₁ 220.1701; found m/z 220.1698 [M+H]⁺. Anal. Calc'd for C₁₄H₂₁NO.HCl: C, 65.74; H, 8.67; N, 5.48; found: C, 65.62; H, 8.53; N, 5.42. [α]²³_{D}=15.6, c=1.17 in CH₃OH.

### EXAMPLE 58

### COUPLING OF CHIRAL AMINE WITH EPOXIDE. PREPARATION OF TERT-BUTYL (1S,2R)-1-(3,5-DIFLUOROBENZYL)-2-HYDROXY-3-{[(4S)-6-NEOPENTYL-3,4-DIHYDRO-2H-CHROMEN-4-YL]AMINO}PROPYLCARBAMATE

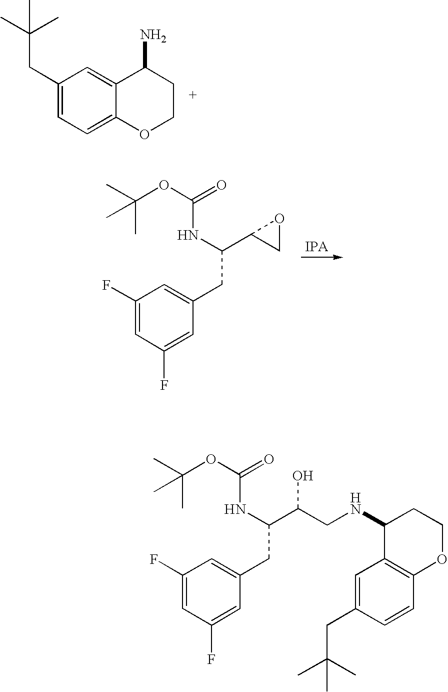

The above compound was prepared essentially according to the method of Example 50, step 3; it was obtained as a white foam. R_{f}=0.25 (in 3% MeOH in CHCl₃ with 1 mL of NH₄OH per liter). HRMS (ESI+) calc'd for C₂₉H₄₀N₂O₄F₂ m/z 519.3034 [M+H]⁺; found 519.3057.

### EXAMPLE 59

### ALTERNATIVE PREPARATION OF TERT-BUTYL (1S,2R)-1-(3,5-DIFLUOROBENZYL)-2-HYDROXY-3-{[(4S)-6-NEOPENTYL-3,4-DIHYDRO-2H-CHROMEN-4-YL]AMINO}PROPYLCARBAMATE

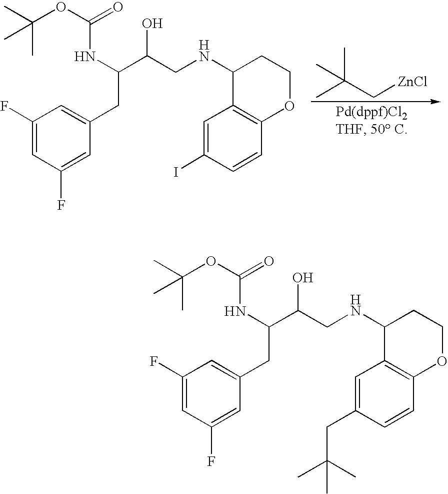

Tert-butyl (1S,2R)-1-(3,5-difluorobenzyl)-2-hydroxy-3-{[(4S)-6-iodo-3,4-dihydro-2H-chromen-4-yl]amino}propylcarbamate (1.3 g, 2.2 mmol) and Pd(dppf)Cl₂ (0.09 g, 0.1 mmol) were added to a neopentyl zinc chloride THF solution (prepared as previously described) (51 mL, 11 mmol, 0.2 M in THF) under N₂(g) at room temperature. The reaction mixture was stirred at room temperature for 12 h and then heated to 50° C. for 8 h. The reaction was cooled to room temperature then quenched with 20 mL of aqueous NH₄Cl and extracted with EtOAc. The combined organic layers were dried (sodium sulfate) and concentrated in vacuo to yield a brown oil. The residue was dissolved in CH₂Cl₂ and absorbed onto 6 g of silica gel. Flash chromatography (3-5% MeOH/CHCl₃ with 20 drops of NH₄OH/L, Biotage 40M) yielded the desired product, which was identical to the material prepared by the previously described methods.

### EXAMPLE 60

### ALTERNATIVE PREPARATION OF N-((1S,2R)-1-(3,5-DIFLUOROBENZYL)-2-HYDROXY-3-{[(4S)-6-NEOPENTYL-3,4-DIHYDRO-2H-CHROMEN-4-YL]AMINO}PROPYL)ACETAMIDE

The above compound is prepared essentially according to the method of Example 3, steps 7-8. First, the Boc group was removed to yield the crude amine as a yellow oil. ¹H NMR (400 MHz, CDCl₃) δ 7.00 (d, J=2.07 Hz, 1 H), 6.95 (dd, J=8.29, 2.28 Hz, 1 H), 6.78-6.68 (m, 4 H), 4.26 (m, 2 H), 3.82 (appt, J=4.15 Hz, 1 H), 3.57 (ddd, J=8.60, 5.29, 3.52 Hz, 1 H), 3.13 (ddd, J=9.89, 5.55, 3.73 Hz, 1 H), 3.07 (dd, J=11.82, 3.52 Hz, 1 H), 2.96 (dd, J=13.58, 3.42 Hz, 1 H), 2.83 (dd, J=11.71, 8.60 Hz, 1 H), 2.53 (dd, J=13.58, 9.85 Hz, 1 H), 2.44 (s, 2 H), 2.14-1.99 (m, 2 H), 0.91 (s, 9 H).

Second, the crude amine was acylated. Flash chromatography (3.5% MeOH/CHCl₃ with 1 mL of NH₄OH per liter), Biotage 40L, yielded the desired product as a white powder. This material was spectroscopically identical to the N-((1S,2R)-1-(3,5-difluorobenzyl)-2-hydroxy-3-{[(4S)-6-neopentyl-3,4-dihydro-2H-chromen-4-yl]amino}propyl)acetamide prepared by previous methods.

### EXAMPLE 61

### ALTERNATIVE PREPARATION OF N-((1S,2R)-1-(3,5-DIFLUOROBENZYL)-2-HYDROXY-3-{[(4S)-6-NEOPENTYL-3,4-DIHYDRO-2H-CHROMEN-4-YL]AMINO}PROPYL)ACETAMIDE

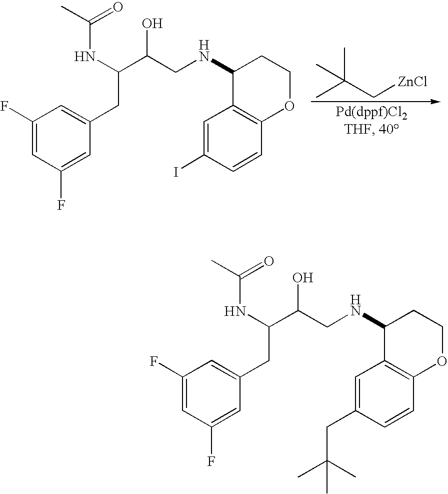

The above compound was prepared essentially according to the procedure of Example 51, step 5. The resulting residue was dissolved in CH₂Cl₂ and absorbed onto 6 g of silica gel. Flash chromatography (3-5% MeOH/CHCl₃ with 20 drops of NH₄OH/L, Biotage 40M) yielded two fractions. The first fraction one yielded 650 mg of the desired product that was 93% pure by analytical HPLC. The second fraction (430 mg) was a 60:40 mixture of the desired product and the dehalogenated compound. The first fraction was re-subjected to preparative reverse phase HPLC (1% TFA in water/0.6% TFA in CH₃CN) to yield 500 mg (38%) of a white powder after neutralization. This material was spectroscopically identical to the N-((1S,2R)-1-(3,5-difluorobenzyl)-2-hydroxy-3-{[(4S)-6-neopentyl-3,4-dihydro-2H-chromen-4-yl]amino}propyl)acetamide prepared by previous methods.

### EXAMPLE 62

### PREPARATION OF THE HCL SALT OF N-((1S,2R)-1-(3,5-DIFLUOROBENZYL)-2-HYDROXY-3-{[(4S)-6-NEOPENTYL-3,4-DIHYDRO-2H-CHROMEN-4-YL]AMINO}PROPYL)ACETAMIDE

The free base from Example 61 (0.5 g, 1.08 mmol) was dissolved in MeOH (10 mL) and treated with HCl/Et₂O (2.5 mL, 1.0 M). The solution was stirred at room temperature for 10 min then the solvent was removed in vacuo to yield a clear glass. The glass was triturated with Et₂O to yield 536 mg of a white solid that was dried in vacuo at 40° C. for 48 h. Anal Calc'd for C₂₆H₃₄F₂N₂O₃.HCl.0.5 H₂O, C, 61.71; H, 7.17; N, 5.54. Found C, 61.69; H, 7.31; N, 5.64. HRMS (ESI+) calc'd for C₂₆H₃₄N₂O₃F₂ m/z 461.2615 [M+H]⁺. Found 461.2627.

### EXAMPLE 63

### PREPARATION OF N-(1S,2R)-1-(3-FLUOROBENZYL)-2-HYDROXY-3-{[(4S)-6-NEOPENTYL-3,4-DIHYDRO-2H-CHROMEN-4-YL]AMINO}PROPYL)ACETAMIDE

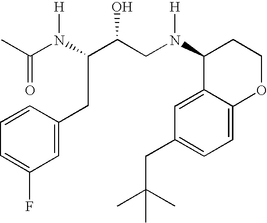

### Step 1: tert-Butyl (1S,2R)-1-(3-fluorobenzyl)-2-hydroxy-3-{[(4S)-6-neopentyl-3,4-dihydro-2H-chromen-4-yl]amino}propylcarbamate

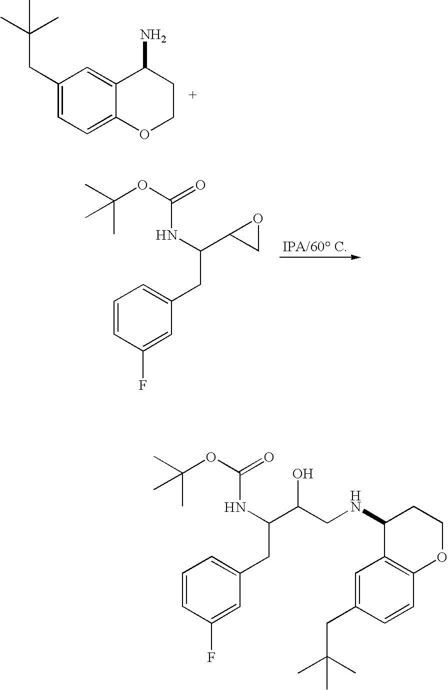

The above product was prepared essentially according to the method of Example 50, step 3. The crude product was then purified by flash chromatography (3% MeOH/CHCl₃). HRMS (ESI+) calc'd for C₂₉H₄₁N₂O₄F m/z 501.3128 [M+H]⁺; found 501.3150.

### Step 2: N-((1S,2R)-1-(3-fluorobenzyl)-2-hydroxy-3-{[(4S)-6-neopentyl-3,4-dihydro-2H-chromen-4-yl]amino}propyl)acetamide

The above compound was prepared essentially according to the method of Example 3, steps 7-8. The crude product was dissolved in MeOH and purified by reverse phase preparatory HPLC. HRMS (ESI+) calc'd for C₂₆H₃₅N₂O₃F m/z 443.2710 [M+H]⁺; found 443.2710.

### EXAMPLE 64

### PREPARATION OF N-((1S,2R)-1-BENZYL-2-HYDROXY-3-{[(4S)-6-NEOPENTYL-3,4-DIHYDRO-2H-CHROMEN-4-YL]AMINO}PROPYL)ACETAMIDE

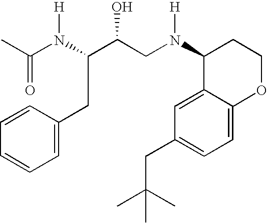

### Step 1: tert-Butyl (1S,2R)-1-benzyl-2-hydroxy-3-{[(4S)-6-neopentyl-3,4-dihydro-2H-chromen-4-yl]amino}propylcarbamate

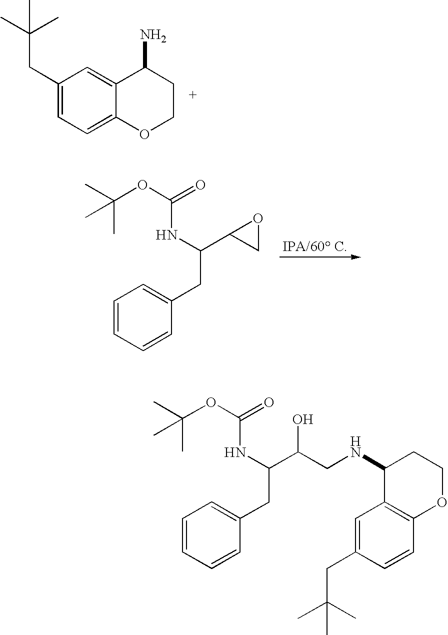

The above compound was prepared essentially according to the method of Example 50, step 3. The resulting crude product was purified by preparative HPLC (1% TFA in water/0.6% TFA in CH₃CN). HRMS (ESI+) calc'd for C₂₉H₄₂N₂O₄ m/z 483.3222 [M+H]⁺; found 483.3219.

### Step 2: N-((1S,2R)-1-benzyl-2-hydroxy-3-{[(4S)-6-neopentyl-3,4-dihydro-2H-chromen-4-yl]amino}propyl)acetamide

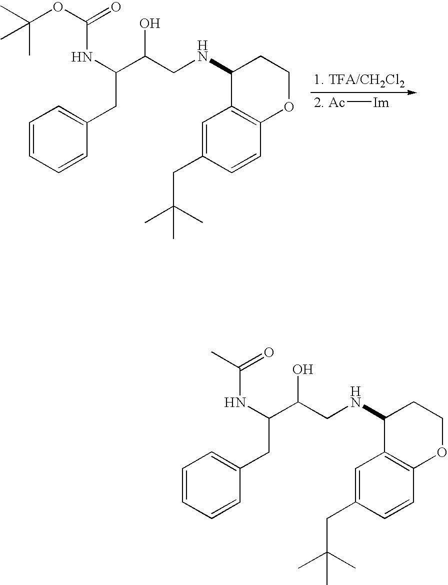

The above compound is prepared essentially according to the method of Example 3, steps 7-8. The resulting crude product was dissolved in MeOH (5 mL) and purified by reverse phase preparatory HPLC which yielded a white powder. HRMS (ESI+) calc'd for C₂₆H₃₆N₂O₃ m/z 425.2804 [M+H]+; found 425.2801.

### EXAMPLE 65

### PREPARATION OF N-((1S,2R)-1-(3,5-DIFLUOROBENZYL)-2-HYDROXY-3-{[(4S)-6-ISOPROPYL-3,4-DIHYDRO-2H-CHROMEN-4-YL]AMINO}PROPYL)ACETAMIDE

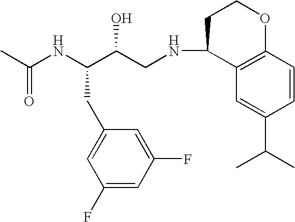

### Step 1: 6-isopropyl-2,3-dihydro-4H-chromen-4-one

A CH₂Cl₂ (350 mL) solution of 1-isopropyl-4-methoxy benzene (25 g, 166 mmol) and 3-chloro-propionyl chloride (21 mL, 216 mmol), at room temperature, was treated with AlCl₃ (33 g, 249 mmol) in 1-2 g portions over a 1 h period. Stirring was maintained at room temperature for 24 h, then the mixture was poured onto crushed ice, and then conc. HCl (30 mL) was added. The mixture was diluted with CH₂Cl₂ (300 mL), washed with 2 N NaOH, dried (magnesium sulfate), and concentrated in vacuo a pale yellow oil. Flash chromatography (10% EtOAc/Heptanes) yields 6-isopropyl-2,3-dihydro-4H-chromen-4-one (7.5 g, 24%). R_{f}=0.3. HRMS (ESI+) calc'd for C₁₂H₁₄O₂ m/z 191.1072 [M+H]⁺; found 191.1071.

### Step 2: 6-isopropylchroman-4-ol

The above compound was prepared essentially according to the method of Example 50, step 1; it was obtained as a white solid. HRMS (ESI+) calc'd for C₁₂H₁₆O₂ m/z 192.1150 [M+H]⁺; found 192.1152.

### Step 3: 6-isopropyl-3,4-dihydro-2H-chromen-4-ylamine

The above compound was prepared essentially according to the method of Example 50, step 2. First the azide was prepared as a yellow oil (7.53 g, 86% crude yield. HRMS calc'd for C₁₂H₁₅N₃O+H1 217.1215, found 217.1218. Second, the azide was reduced with 1.OM Me₃P in THF (42.00 mL, 41.59 mmol). The resulting amine was obtained as a yellow oil (3.5 g, 53% crude yield). HRMS calc'd for C₁₂H₁₇NO+H1 192.1388, found 192.1384. The crude racemic amine was purified and resolved using chiral preparative HPLC (5% EtOH/heptanes, 0.1% DEA) using a Chiralpak AD column. Obtained 1.5 g of (+)-(4R)-6-isopropyl-chromen-4-ylamine retention time 15.5 min. [α]_{D}=4.2 (c=2.0 in MeOH) and 1.5 g of (−)-(4S)-6-isopropyl-chromen-4-ylamin retention time 18.3 min. [α]_{D}=−3.9 (c=2.0 in MeOH). ¹H NMR as the HCl salt (300 MHz, CD₃OD) δ 1.25 (d, J=6 Hz, 6 H), 2.15 (m, 1 H), 2.38 (m, 1 H), 2.89 (m, 1 H), 4.27 (m, 2 H), 4.55 (t, J=6 Hz, 1 H), 6.83 (d, J=9 Hz, 1 H), 7.19 (dd, J=3, 9 Hz, 1 H), 7.25 (d, J=3 Hz, 1 H).

### Step 4: tert-Butyl (1S,2R)-1-(3,5-difluorobenzyl)-2-hydroxy-3-{[(4S)-6-isopropyl-3,4-dihydro-2H-chromen-4-yl]amino}propylcarbamate

The above compound was prepared essentially according to the method of Example 50, step. 3. The crude material was used in the next reaction without purification. ¹H NMR (crude-DMSO-d₆) δ 7.75 (d, J=9 Hz, 1 H), 7.14 (br s, 1 H), 7.02 (m, 2 H), 6.9 (m, 1 H), 6.68 (d, J=9 Hz, 1 H), 5.3 (br s, 2 H), 4.22 (m, 1 H), 4.12 (m, 1 H), 3.9 (m, 1 H), 3.68 (m, 1 H), 3.50 (m, 1 H), 3.02 (dd, J=11, 3 Hz, 1 H), 2.78 (sept, J=7 Hz, 1 H), 2.67 (s, 1 H), 2.57 (dd, J=4, 10 Hz, 1 H), 1.59 (s, 9 H), 1.14 (d, J=7 Hz, 6 H). LRMS (m/z) M+H: 490.3.

### Step 5: N-((1S,2R)-1-(3,5-difluorobenzyl)-2-hydroxy-3-{[(4S)-6-isopropyl-3,4-dihydro-2H-chromen-4-yl]amino}propyl)acetamide

The product from step 4 was converted into the above compound essentially according to the method of Example 3, steps 7-8. First, the free amine was obtained as a glassy solid/foam. ¹H NMR (crude-CDCl₃) δ 7.75 (d, J=9 Hz, 1 H), 7.14 (br s, 1 H), 7.02 (m, 2 H), 6.9 (m, 1 H), 6.68 (d, J=9 Hz, 1 H), 4.4 (br s, 2 H), 4.12 (m, 1 H), 3.9 (m, 1 H), 3.68 (m, 1 H), 3.50 (m, 1 H), 3.32 (m, 1 H), 3.02 (dd, J=11, 3 Hz, 1 H), 2.78 (sept, J=7 Hz, 1 H), 2.67 (s, 1 H), 2.57 (dd, J=4, 10 Hz, 1 H), 1.11 (d, J=7 Hz, 6 H). LRMS (m/z) M+H:390.2.

Second, the amine was acylated to yield the acetamide as an oil, which was purified by prep-HPLC. HRMS (ESI+) calc'd for C₂₄H₃₀F₂N₂O₃ m/z 433.2303 [M+H]⁺; found 433.2307.

The same procedure using (+)-(4R)-6-isopropyl-chromen-4-ylamine results in the epimer N-((1S,2R)-1-(3,5-difluorobenzyl)-2-hydroxy-3-{[(4R)-6-isopropyl-3,4-dihydro-2H-chromen-4-yl]amino}propyl)acetamide. ¹H NMR (DMSO-d₆) δ 7.76 (d, J=9 Hz, 1 H), 7.01 (m, 2 H), 7.14 (d, J=2 Hz, 1 H), 6.99 (dd, J=8.5, 2.Hz, 1 H), 6.91 (m, 1 H), 6.65 (d, J=8.5 Hz, 1 H), 4.96 (d, J=6 Hz, 1 H), 4.2 (dt, J=10, 3.4 Hz, 1 H), 4.1 (m, 1 H), 3.99 (m, 1 H), 3.64 (br s, 1 H), 3.47 (m, 1 H), 3.0 (dd, J=14, 3 Hz, 1 H), 2.78 (sept, J=8 Hz, 1 H), 2.75 (m, 1 H), 2.6 (m, 2 H), 1.86 (m, 3 H), 1.7 (s, 3 H), 1.16 (d, J=7 Hz, 6 H). HRMS (ESI+) calc'd for C₂₄H₃₀F₂N₂O₃ m/z 433.2303 [M+H]⁺; found 433.2301.

### EXAMPLE 66

### PREPARATION OF N-((1S,2R)-1-(3,5-DIFLUOROBENZYL)-2-HYDROXY-3-{[(4S)-6-IODO-3,4-DIHYDRO-2H-CHROMEN-4-YL]AMINO)PROPYL)-2-HYDROXY-2-METHYLPROPANAMIDE

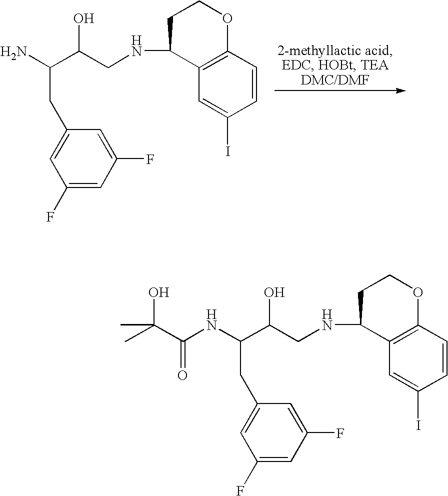

(2R,3S)-3-Amino-4-(3,5-difluorophenyl)-1-{[(4S)-6-iodo-3,4-dihydro-2H-chromen-4-yl]amino}butan-2-ol (1 equiv) was combined with 2-methylacetic acid, (1.25 equiv), EDC (1.5 equiv) and HOBt (1.5 equiv) in DMF/DCM (1:1, 10 mL). The reaction mixture was treated with Et₃N and stirred at room temperature for 6 h. After consumption of the amine, the reaction was poured onto EtOAc, washed with 1M HCl, dried (magnesium sulfate), and concentrated to give an oil which was purified by reverse phase preparative HPLC. HRMS (ESI+) calc'd for C₂₃H₂₇F₂IN₂O₄ m/z561.1063 [M+H]⁺; found 561.1047.

### EXAMPLE 67

### PREPARATION OF N-((1S,2R)-1-(3,5-DIFLUOROBENZYL)-2-HYDROXY-3-{[(4S)-6-IODO-3,4-DIHYDRO-2H-CHROMEN-4-YL]AMINO}PROPYL)-1-HYDROXYCYCLOPROPANE CARBOXAMIDE

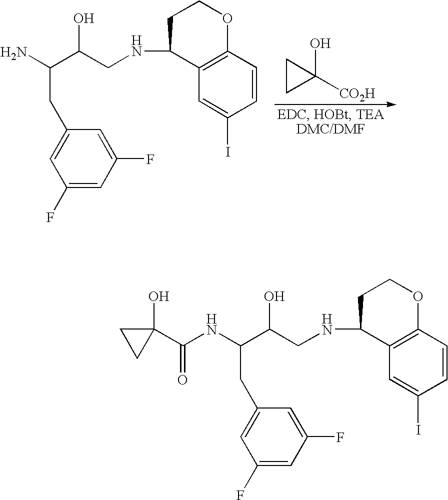

The above compound is prepared using the basic methodology described in Example 66. HRMS (ESI+) calc'd for C₂₃H₂₅F₂IN₂O₄ T/z 559.0907 [M+H]⁺; found 559.0903.

### EXAMPLE 68

### PREPARATION OF N-((1S,2R)-1-(3,5-DIFLUOROBENZYL)-2-HYDROXY-3-{[(4S)-6-IODO-3,4-DIHYDRO-2H-CHROMEN-4-YL]AMINO}PROPYL)METHANESULFONAMIDE

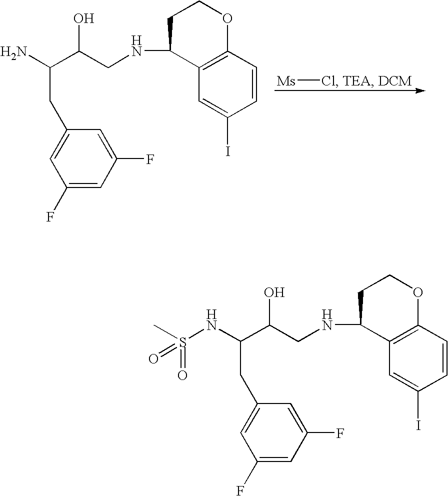

(2R,3S)-3-Amino-4-(3,5-difluorophenyl)-1-{[(4S)-6-iodo-3,4-dihydro-2H-chromen-4-yl]amino}butan-2-ol (1 equiv) was dissolved in DCM with TEA (2 equiv) then cooled to 0° C. and treated with MsCl (1.25 equiv) while stirring. The reaction mixture was removed from the cold bath, brought to room temperature, then quenched with MeOH and concentrated. The residue was dissolved in EtOAc and washed with 1M HCl. The organics were dried, concentrated, and chromatographed over silica gel. ¹H NMR (CD₃OD) δ 7.74 (d, J 2.0 Hz, 1 H), 7.53 (dd, J=2.0, 8.7 Hz, 1 H), 6.88 (m, 2 H), 6.77 (m, 1 H), 6.67 (d, J=8.7 Hz, 1 H), 4.23-4.39 (m, 2 H), 4.25 (br m, 1 H), 4.12 (m, 1 H), 3.87 (td, J=3.1, 7.8 Hz, 1 H), 3.29 (dd, J=3.5, 13.9 Hz, 1 H), 3.11 (s, 3H), 3.05 (dd, J=3.2, 12.7 Hz, 1 H), 2.98 (dd, J=7.9, 12.6 Hz, 1 H), 2.74 (dd, J=11.0, 13.9 Hz, 1 H), 2.14 (br m, 2 H). MS (ESI+) calc'd for C₂₀H₂₃F₂1N₂O₄S m/z553.38, [M+H]⁺; found 553.4.

### EXAMPLE 69

### PREPARATION OF (1S,2R)-1-(3,5-DIFLUOROBENZYL)-2-HYDROXY-3-{[(4S)-6-NEOPENTYL-3,4-DIHYDRO-2H-CHROMEN-4-YL]AMINO}PROPYLFORMAMIDE

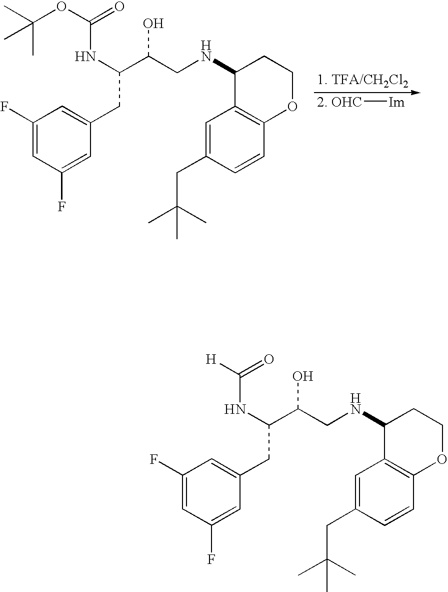

The Boc protected amine (1 equiv) was dissolved in 10:1 DCM:TFA (to 0.1M) for 3 h at room temperature. The reaction mixture was concentrated and the residue partitioned between EtOAc and 1M NaOH. The aqueous layer was removed and the organics washed with brine (50 mL), dried (magnesium sulfate) and concentrated to a glassy solid/foam. LRMS (m/z) M+H:418.5. This was dissolved in CH₂Cl₂ (to 0.1M), cooled to 0° C. and treated with formyl imidizole (1.25 equiv). The reaction was removed from the cold bath, then stirred for 2 h at room temperature. When the reaction was complete, the mixture was concentrated, dissolved in MeOH (1.5 mL), and purified by reversed phase preparative HPLC (2 in. column) to give a film which scraped down to a white powder. ¹H NMR (DMSO-d₆) δ 8.46 (br s, 1 H), 7.75 (d, J=9 Hz, 1 H), 7.14 (br s,1 H), 7.02 (m, 2 H), 6.91 (m, 1 H), 6.69 (d, J=9 Hz, 1 H), 5.0 (br s, 2 H), 4.21 (m, 1 H), 4.09 (m, 1 H), 3.94 (m, 1 H), 3.72 (m, 1 H), 3.43 (m, 1 H), 3.08 (dd, J=11, 3 Hz, 1 H), 2.77 (s, 2 H), 2.57 (dd, J=4, 10 Hz, 1 H), 1.69 (s, 3 H), 1.04 (s, 9H). MS (ESI+) for C₂₅H₃₂F₂N₂O₃ m/z 446.54 [M+H]+; found 446.3.

### EXAMPLE 70

### PREPARATION OF N-{(1S,2R)-1-(3,5-DIFLUOROBENZYL)-2-HYDROXY-3-[(4-METHYL-6-NEOPENTYL-3,4-DIHYDRO-2H-CHROMEN-4-YL)AMINO]PROPYL}ACETAMIDE

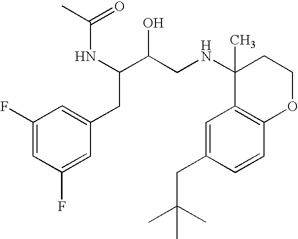

### Step 1: 6-Iodo-2,3-dihydro-4H-chromen-4-one

PCC (15.2 g, 70.6 mmol) as a solid was added to a CH₂Cl₂ (300 mL) suspension of 6-iodo-4-chromenol (15 g, 54.3 mmol) and 30 g of silica gel at room temperature. The mixture was stirred at room temperature for 3 h at which time TLC (20% EtOAc/hexanes) indicated complete reaction. The reaction mixture was filtered through a silica gel plug and the filtrate concentrated in vacuo to yield 14.9 g (95%) of 6-iodo-2,3-dihydro-4H-chromen-4-one as a white solid consistent with the literature report (Synthesis, 1997, 23-25). HRMS (ESI+) calc'd for C₉H₇IO₂ m/z 273.9492; found 273.9500.

### Step 2: 6-Iodo-4-methylchroman-4-ol

CeCl₃ (4.9 g, 19.8 mmol) was dried in vacuo at 140° C. for 3 h and then slurried with dry THF (100 mL) for 1 h. The white suspension was chilled to −78° C., then MeLi.LiBr (14.2 mL, 21.4 mmol) was added over 15 min. The mixture was stirred for 30 min, then a THF (20 mL) solution of 6-iodo-2,3-dihydro-4H-chromen-4-one was added dropwise via syringe. After 30 min, TLC (15% EtOAc/hexanes) indicated the reaction was complete. The mixture was treated with NH₄Cl (aq.) (30 mL), diluted with water (150 mL), extracted with EtOAc, and dried (sodium sulfate). The sodium sulfate was removed by filtration and the filtrate concentrated in vacuo to yield 6-iodo-4-methylchromen-4-ol as an off white solid 4.7 g (95%). HRMS (ESI+) calc'd for C₁₀H₁₁IO₂ m/z 289.9806 [M+H]⁺; found 289.9803.

### Step 3: 6-iodo-4-methylchroman-4-amine

TFA (1.3 mL, 17.2 mmol) in 10 mL of CHCl₃ was added to a mixture of 6-iodo-4-methylchroman-4-ol (1.0 g, 3.4 mmol) and NaN₃ (0.7 g, 10.3 mmol) in CHCl₃ (15 mL), at 0° C. dropwise via addition funnel. The addition was carried out over 2 h and stirring continued for an additional 2 h at 0° C. The mixture was warmed to room temperature and stirred overnight. The mixture was diluted with 30 mL of water and extracted with CH₂Cl₂. The organic layer was dried (sodium sulfate) and concentrated in vacuo to yield 4-azido-6-iodo-4-methylchromen as a yellow oil. ¹H NMR (400 MHz, CDCl₃) δ 7.65 (d, J=2.07 Hz, 1 H), 7.50 (dd, J 8.71, 2.07 Hz, 1 H), 6.66 (d, J=8.71 Hz, 1 H), 4.27 (m, 2 H), 2.06 (m, 2 H), 1.68 (s, 3 H). MS (ESI+) for C,OH,OIN₃0 m/z273.0 [M+H]⁺ loss of azide. The crude azide was dissolved in THF (15 mL), then trimethylphosphine (4 mL, 1.0 M in THF) was added at room temperature. After 15 min, 3 mL of water was added and stirring continued at room temperature for 2 h until complete as indicated by LC/MS. The solvent was removed in vacuo and the residue diluted with water (75 mL), extracted with CH₂Cl₂, dried (sodium sulfate) filtered, and concentrated in vacuo to yield 6-iodo-4-methylchromen-4-amine (0.900 g, 91%) as a yellow oil. This material was used in the next step without purification. ¹H NMR (400 MHz, CDCl₃) δ 7.77 (d, J=2.07 Hz, 1 H), 7.40 (dd, J=8.60, 2.18 Hz, 1 H), 6.59 (d, J=8.50 Hz, 1 H), 4.25 (m, 2 H), 2.01 (m, 2 H), 1.53 (s, 3 H). MS (ESI+) for C₁₀H₁₂INO m/z 273.2 [M+H]⁺ loss of NH₃.

### Step 4: tert-butyl (1S,2R)-1-(3,5-difluorobenzyl)-2-hydroxy-3-[(6-iodo-4-methyl-3,4-dihydro-2H-chromen-4-yl)amino]propylcarbamate

The above compound was prepared essentially according to the method of Example 50, step 3. The resulting crude material was dissolved in CH₂Cl₂, absorbed onto 7.8 g of silica gel, and purified by flash chromatography (Biotage 40 M column, eluent: using 50% EtOAc/Heptanes). Three fractions were obtained. The final fraction was recovered amine. Obtained 0.500 g of each of the following diastereomers overall yield from epoxide 83%.
Diastereomer A: ¹H NMR (400 MHz, CDCl₃) δ 7.67 (bs, 1 H), 7.42 (dd, J=8.50, 2.07 Hz, 1 H), 6.71 (m, 3 H), 6.59 (d, J=8.50 Hz, 1 H), 4.52 (d, J=9.12 Hz, 1 H), 4.35 (m, 1 H), 4.21 (m, 1 H), 3.82 (m, 1 H), 3.42 (m, 1 H), 3.06 (m, 1 H), 2.81 (dd, J=14.3, 8.7 Hz, 1 H), 2.62 (m, 2 H), 2.26 (m, 1 H), 1.84 (m, 1 H), 1.40 (m, 2 H), 1.35 (m, 12 H). HRMS (ESI+) for C₂₅H₃₁N₂O₄F₂I+1H calc'd for 589.1376 m/z found 589.1397 [M+H]⁺.
Diastereomer B: ¹H NMR (400 MHz, CDCl₃) δ 7.65 (d, J=2.07 Hz, 1 H), 7.44 (d, J=8.50 Hz, 1 H), 6.71 (m, 3 H), 6.67 (d, J=8.71 Hz, 1 H), 4.54 (bs, 1 H), 4.34 (m, 1 H), 4.16 (m, 1 H), 3.77 (m, 1 H), 3.48 (m, 1 H), 3.10 (m, 1 H), 2.75 (m, 1 H), 2.75 (m, 1 ), 2.62 (m, 2 H), 2.24 (m, 1 H), 1.93 (m, 1 H), 1.60 (m, 2 H), 1.42 (s, 9 H), 1.39 (s, 3 H). HRMS (ESI+) for [C₂₅H₃₁N₂O₄F₂I+H] calc'd for m/z 589.1376; found 589.1375 [M+H]⁺.

### Step 5: N-{(1S,2R)-1-(3,5-Difluorobenzyl)-2-hydroxy-3-[(6-iodo-4-methyl-3,4-dihydro-2H-chromen-4-yl)amino]propyl}acetamide

25 mL of 20% TFA/CH₂Cl₂ was added to a CH₂Cl₂ (5 mL) solution of tert-butyl (1S,2R)-1-(3,5-difluorobenzyl)-2-hydroxy-3-[(6-iodo-4-methyl-3,4-dihydro-2H-chromen-4-yl)amino]propylcarbamate (Diastereomer B). (0.47 g, 0.79 mmol), at room temperature. The mixture was stirred for 30 min. The solvent was removed in vacuo and the residue dissolved in CH₂Cl₂ (75 mL), washed with aqueous NaHCO₃ and brine, dried (sodium sulfate), filtered, and concentrated in vacuo to yield a white foam. The residue was dissolved in CH₂Cl₂ (5 mL), chilled to 0° C., then Et₃N (0.24 mL, 1.7 mmol) and acetyl imidazole (0.10 g, 0.90 mmol) were added. The mixture was then warmed to room temperature, stirred overnight, diluted with CH₂Cl₂ (25 mL), washed with water and brine, dried (sodium sulfate), and concentrated in vacuo to yield a white foam (0.35 g, 84%) after flash chromatograph 5% MeOH/CHCl₃ (Biotage 40 S). R_{f}=0.29. HRMS (ESI+) calc'd for C₂₂H₂₅N₂O₃IF₂+1 H calc'd +nZ 531.0958; found 531.0958 [M+H]⁺.

The same procedure for diastereomer A yields 0.28 g (70%) of the epimer. ¹H NMR (400 MHz, DMSO-d₆) δ 7.75 (d, J=2.28 Hz, 1 H), 7.36 (dd, J=8.71, 2.28 Hz, 1 H), 6.79 (m, 3 H), 6.57 (d, J=8.50 Hz, 1 H), 4.31 (m, 1 H), 4.17 (m, 1 H), 4.08 (m, 1 H), 3.51 (m, 1 H), 3.11 (dd, J=14.1, 3.73 Hz, 1 H), 2.62 (dd, J=14.1, 10.4 Hz, 1 H), 2.52 (m, 1 H), 2.45 (dd, J=11.9, 3.63 Hz, 1 H), 2.25 (m, 1 H), 1.79 (s, 3 H), 1.74 (m, 1 H), 1.47 (s, 3 H). Anal. Calc'd for C₂₂H₂₅F₂IN₂O₃; C, 49.82; H, 4.75; N, 5.28; found C, 49.87; H, 4.94; N, 5.05.

### Step 6: N-{(1S,2R)-1-(3,5-Difluorobenzyl)-2-hydroxy-3-[(4-methyl-6-neopentyl-3,4-dihydro-2H-chromen-4-yl)amino]propyl}acetamide

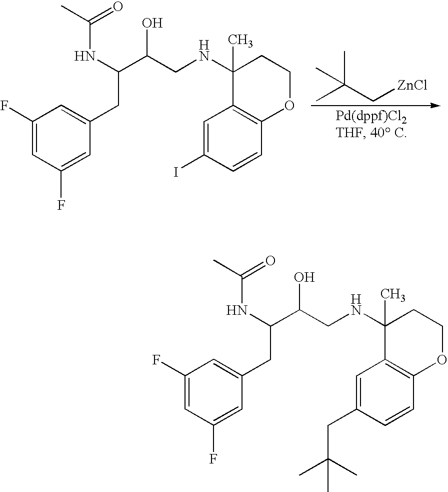

Neopentyl zinc chloride (3.7 mL of a 0.5 M solution, 1.85 mmol), prepared as previously described, was added to a 20 mL serum capped vial containing N-{(1S,2R)-1-(3,5-difluorobenzyl)-2-hydroxy-3-[(6-iodo-4-methyl-3,4-dihydro-2H-chromen-4-yl)amino]propyl}acetamide (0.20 g, 0.37 mmol) and Pd(dppf)Cl₂ (0.015 g, .018 mmol) under N₂(g). The mixture was shaken on an orbital shaker for 12 h at which time LC/MS indicated only a trace of the desired compound. An additional 5 equiv of the zinc reagent and another 5 mol % of catalyst were added and the reaction mixture was warmed to 40° C. After 6 h LC/MS indicated complete consumption of starting material. The reaction mixture was quenched with NH₄Cl and extracted with EtOAc, dried (sodium sulfate), filtered, and concentrated in vacuo to yield a light brown solid (150 mg) after flash chromatography (4% MeOH/CHCl₃ Biotage 40S). This material was subjected to a final reverse phase preparative column (1% TFA in H₂O/0.6% TFA in CH₃CN) to yield 50 mg of a light yellow solid. This material was dissolved in 4 mL of CH₂Cl₂ and treated with 0.5 g of 3-mercaptopropyl functionalized silica gel and stirred at room temperature for 30 min. The mixture was filtered through Celite® to remove the resin, then the filtrate was concentrated in vacuo to yield a white powder (44 mg, 20%). ¹H NMR (400 MHz, DMSO-d₆) δ 7.08 (d, J=2.07 Hz, 1 H), 6.87 (dd, J=8.29, 2.07 Hz, 1 H), 6.78 (m, 3 H), 6066 (d, J=8.29 Hz, 1 H), 4.27 (m, 1 H), 4.12 (m, 1 H), 4.04 (m, 1 H), 3.54 (m, 1 H), 3.06 (dd, J=13.99, 3.63 Hz, 1 H), 2.56 (m, 2 H), 2.45 (bs, 2 H), 2.37 (dd, J=11.82, 7.67 Hz, 1 H), 2.25 (m, 1 H), 1.81 (s, 3 H), 1.78 (m, 1 H); 1.49 (s, 3 H), 0.91 (s, 9 H). MS (ESI+) for C₂₇H₃₆N₂O₃F₂ m/z475.2772 [M+H]⁺; found, 475.2774.

The same procedure yields 0.049 g (28%) of the epimer ¹H NMR (400 MHz, DMSO-d₆) δ 7.17 (d, J=2.07 Hz, 1 H), 6.87 (dd, J=8.29, 2.07 Hz, 1 H), 6.77 (m, 3 H), 6.66 (d, J=8.29 Hz, 1 H), 4.27 (m, 1 H), 4.11 (m, 2 H), 3.53 (m, 1 H), 3.06 (dd, J=14.10, 3.52 Hz, 1 H), 2.53 (m, 3 H), 2.43 (s, 2 H), 2.27 (m, 1 H), 1.78 (m, 4 H), 1.49 (s, 3 H), 0.90 (s, 9 H). MS (ESI+) calc'd for C₂₇H₃₆N₂O₃F₂ m/z 475.2772 [M+H]⁺; found, 475.2788.

### EXAMPLE 71

### ALTERNATIVE SYNTHESIS OF 4-METHYL-6-NEOPENTYLCHROMAN-4-OL

### Step 1: 6-neopentylchroman-4-ol

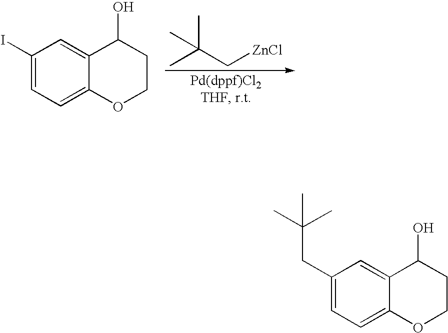

6 mL of anhydrous THF was added to a flame dried round bottom flask containing 6-iodo-chroman-4-ol (3.0 g, 10.8 mmol) and Pd(dppf)Cl₂ (0.44 g, 0.54 mmol) and the mixture was chilled to 0° C. The mixture was treated with neopentyl zinc chloride (prepared as previously described) (50 mL, 30 mmol, 0.6 M in THF) and stirred under N₂(g) at room temperature for 19 h followed by 5 h at 50° C. (oil bath). The reaction was cooled to room temperature and quenched with NH₄Cl and extracted with EtOAc, then the organic layer was dried (sodium sulfate), filtered, and concentrated in vacuo to 1.9 g (79%) of a white solid after flash chromatography (10% EtOAc/heptanes, Biotage 40M) R_{f}=0.11. HRMS (ESI+) calc'd for C₁₄H₂O₂ m/z 220.1463 [M+H]⁺; found 220.1460.

### Step 2: 6-neopentyl-2,3-dihydro-4H-chromen-4-one

The alcohol was oxidized to the ketone essentially according to the method of Example 70, step 1; the ketone was obtained as a clear oil. This material was carried forward without further purification. HRMS (ESI+) calc'd for C₁₄H₁₈O₂ m/z 219.1385 [M+H]⁺; found 219.1393.

### Step 3: 4-Methyl-6-neopentylchroman-4-ol

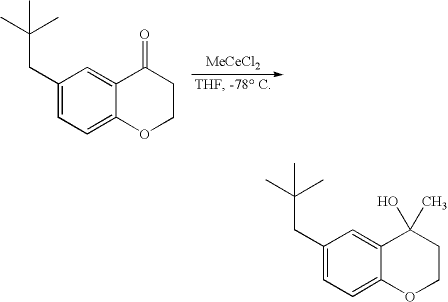

The above compound was prepared essentially according to the method of Example 70, step 2; the product was obtained as a clear oil, which was used without further purification. ¹H NMR (300 MHz, CDCl₃) δ 7.23 (d, J=2.07 Hz, 1 H), 6.95 (dd, J=8.29, 2.26 Hz, 1 H), 6.73 (d, J=8.29 Hz, 1 H), 4.25 (m, 2 H), 2.44 (s, 2 H), 2.09 (m, 2 H), 1.64 (s, 3 H), 0.91 (s, 9 H). MS (ESI+) calc'd for C₁₅H₂₂O₂ m/z 234.2 [M+H]⁺; found 217.3 loss of water.

### EXAMPLE 72

### PREPARATION OF N-((1S,2R)-1-(3,5-DIFLUOROBENZYL)-2-HYDROXY-3-{[(4S)-6-ISOPROPOXY-3,4-DIHYDRO-2H-CHROMEN-4-YL]AMINO}PROPYL)ACETAMIDE

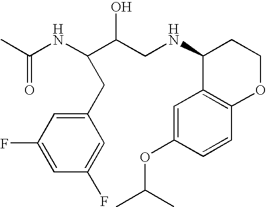

### Step 1: tert-Butyl (4S)-6-(4,4,5,5-tetramethyl-1,3,2-dioxaborolan-2-yl)-3,4-dihydro-2H-chromen-4-ylcarbamate

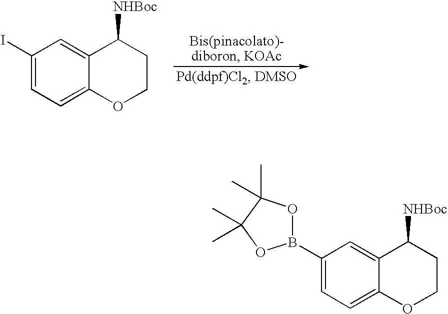

Potassium acetate (2.60 g, 26.4 mmol) followed by [1,1′-bis(diphenylphosphino)ferrocene]dichloropalladium(II) complex with dichloromethane (1:1) (410 mg, 0.5 mmol) was added to a mixture of tert-butyl (4S)-6-iodo-3,4-dihydro-2H-chromen-4-ylcarbamate (3.30 g, 8.8 mmol) and bis(pinacolato)diboron (2.51 g, 9.7 mmol) in methyl sulfoxide (30 mL). The reaction mixture was heated under argon at 80° C. for 2 h and then cooled to room temperature. The reaction mixture was diluted with ethyl ether (100 mL), washed with water and saturated sodium chloride, dried (sodium sulfate), filtered, and concentrated under reduced pressure. Purification by flash column chromatography (silica gel, 10-20% ethyl acetate/hexanes) provided the desired product (3.25 g, 98%): ¹H NMR (300 MHz, CDCl₃) δ 7.72 (s, 1H), 7.62 (dd, J=8.2,1.5 Hz, 1H), 6.80 (d, J=8.2 Hz, 1H), 4.79 (m, 2H), 4.31-4.24 (m, 1H), 4.21-4.15 (m, 1H), 2.14-2.11 (m, 2H), 1.48 (s, 9H), 1.34 (s, 6 H), 1.33 (s, 6 H).

### Step 2: tert-Butyl (4S)-6-hydroxy-3,4-dihydro-2H-chromen-4-ylcarbamate

Sodium hydroxide (6 mL, 1: N, 6 mmol) followed by hydrogen peroxide (10 mL, 30%), was added to a solution of tert-butyl (4S)-6-(4,4,5,5-tetramethyl-1,3,2-dioxaborolan-2-yl)-3,4-dihydro-2H-chromen-4-ylcarbamate (1.09 g, 2.90 mmol) in tetrahydrofuran (10 mL). The reaction mixture was stirred at room temperature for 2 h and then quenched with sodium hydrogen sulfite (5 g in 10 mL of water). The mixture was adjusted to pH 4 with 2 N sodium hydroxide and then extracted with ethyl acetate. The combined extracts were washed with saturated sodium chloride, dried (sodium sulfate), filtered, and concentrated under reduced pressure. Flash chromatography (silica gel, 10-25% ethyl acetate/hexanes) provided 650 mg (85%) of the desired product. ¹H NMR (300 MHz, CDCl₃) δ 7.89 (s, 1H), 6.75 (d, J=2.7 Hz, 1H), 6.72-6.63 (m, 1H), 5.03 (d, J=7.5 Hz, 1 H), 4.77-4.75 (m, 1H), 4.16-4.08 (m, 2H), 2.30 (s, 1H), 2.16-2.13 (m, 1H), 2.05-1.99 (m, 1H), 1.47 (s, 9H).

### Step 3: tert-Butyl (4S)-6-isopropoxy-3,4-dihydro-2H-chromen-4-ylcarbamate

Cesium carbonate (800 mg, 2.45 mmol) followed by 2-bromopropane (360 mg, 2.93 mmol) were added to a solution of the alcohol, from step 2, (325 mg, 1.22 mmol) in acetone (10 mL). The reaction mixture was stirred at 60° C. for 24 h. The solvent was removed under reduced pressure. The residue was diluted with ethyl acetate (100 mL) and water (50 mL). The organic layer was separated and washed with saturated sodium chloride, dried (sodium sulfate), filtered, and concentrated under reduced pressure to provide tert-butyl (4S)-6-isopropoxy-3,4-dihydro-2H-chromen-4-ylcarbamate (340 mg, 90%): ¹H NMR (300 MHz, CDCl₃) δ 6.80 (d, J=2.1 Hz, 1H), 6.77-6.62 (m, 2H), 4.81 (m, 2 H), 4.45-4.35 (m, 1H), 4.23-4.16 (m, 1H), 4.14-4.06 (m, 1H), 2.22-2.14 (m, 1H), 2.05-1.95 (m, 1H), 1.48 (s, 9H), 1.29 (d, J=6.2 Hz, 6H). This material was used in the next step without further purification.

### Step 4: (4S)-6-Isopropoxychroman-4-amine

Hydrochloric acid (2 mL, 4 N in 1,4-dioxane, 8 mmol) was added to a solution of tert-butyl (4S)-6-isopropoxy-3,4-dihydro-2H-chromen-4-ylcarbamate (340 mg, 1.11 mmol) in methanol (2 mL). The reaction mixture was stirred at room temperature for 2 h. The solvent was removed under reduced pressure. The residue was diluted with methylene chloride (50 mL) and water (50 mL). The organic layer was separated and the aqueous layer was extracted with methylene chloride. The combined extracts were washed with saturated sodium chloride, dried (sodium sulfate), filtered, and concentrated under reduced pressure to provide (4S)-6-isopropoxychromen-4-amine (240 mg, 99% crude yield): ¹H NMR (300 MHz, CDCl₃) δ 6.96 (d, J=2.7 Hz, 1H), 6.90-6.86 (m, 1H), 6.80 (d, J=9.0 Hz, 1H), 4.55-4.46 (m, 2H), 4.24-4.17 (m, 2H), 2.40-2.31 (m, 1H), 2.18-2.08 (m, 1H), 1.28 (d, J=6.0 Hz, 6H). This material was used in the next step without further purification.

### Step 5: tert-Butyl (1S,2R)-1-(3,5-difluorobenzyl)-2-hydroxy-3-{[(4S)-6-isopropoxy-3,4-dihydro-2H-chromen-4-yl]amino}propylcarbamate

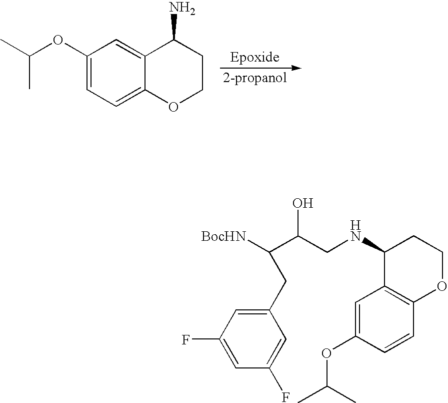

The above compound was prepared essentially according to the method of Example 50, step 3. Flash chromatography of the crude product (silica gel, 20-50% ethyl acetate/hexanes) yielded 95 mg of amine and the desired product (330 mg, 93%): ¹H NMR (300 MHz, CDCl₃) δ 6.84 (s, 1H), 6.79-6.73 (m, 4H), 6.70-6.63 (m, 1H), 4.52 (d, J=9.4 Hz, 1H), 4.45-4.37 (m, 1H), 4.25-4.13 (m, 2H), 3.77-3.69 (m, 2H), 3.45-3.39 (m, 1H), 3.09-3.03 (m, 1H), 2.83-2.75 (m, 3H), 2.05-2.01 (m, 1H), 1.95-1.87 (m, 1H), 1.37 (s, 9H), 1.30 (d, J=6.1 Hz, 6H).

### Step 6: (2R,3S)-3-Amino-4-(3,5-difluorophenyl)-1-{[(4S)-6-isopropoxy-3,4-dihydro-2H-chromen-4-yl]amino}butan-2-ol hydrochloride

Hydrochloric acid (2 mL, 4 N in 1,4-dioxane, 8 mmol) was added to a solution of the product from step 5 (330 mg, 0.65 mmol) in 1,4-dioxane (2 mL). The reaction mixture was stirred at room temperature for 4 h. The solvent was removed under reduced pressure. The residue was triturated with ethyl ether. The resulting white solid was collected by filtration and washed with ethyl ether to provide (2R,3S)-3-amino-4-(3,5-difluorophenyl)-1-{[(4S)-6-isopropoxy-3,4-dihydro-2H-chromen-4-yl]amino}butan-2-ol hydrochloride (302 mg, 97%): ESI MS m/z 407 [C₂₂H₂₈F₂N₂O₃+H]⁺. This material was used in the next step without further purification.

### Step 7: N-((1S,2R)-1-(3,5-difluorobenzyl)-2-hydroxy-3-{[(4S)-6-isopropoxy-3,4-dihydro-2H-chromen-4-yl]amino}propyl)acetamide

Triethylamine (322 mg, 3.15 mmol), followed by 1 acetylimidazole (71 mg, 0.63 mmol), was added to a solution of the product from step 6 (302 mg, 0.63 mmol) in methylene chloride (5 mL). The reaction mixture was stirred at room temperature overnight. The mixture was washed successively with 1N hydrochloric acid, water, saturated sodium bicarbonate and saturated sodium chloride, and dried (sodium sulfate), filtered, and concentrated under reduced pressure. Purification by flash column chromatography (silica gel, 0-5% methanol/methylene chloride provided the desired product (190 mg, 67%) as a white solid: ESI MS m/z 449 [C₂₄H₃₀F₂N₂O₄+H]⁺; HPLC (Phenomenex Luna Cl 8(2) Column, 150×4.6 mm, 5 g; A: 0.05% TFA in 95:5 H₂O/CH₃CN; B: 0.05% TFA in 5:95 H₂O/CH₃CN; Gradient: 10-90% B over 15 min; flow 1.0 mL/min; Detection: 254 nm) 98.7% (AUC), t_{R}=8.69 min. Anal. Calc'd for C₂₄H₃₀F₂N₂O₄: C, 64.27; H, 6.74; N, 6.24; found: C, 64.11; H, 6.65; N, 6.17.

### EXAMPLE 73

### PREPARATION OF N-((1S,2R)-1-(3,5-DIFLUOROBENZYL)-2-HYDROXY-3-{[(4S)-6-HYDROXY-3,4-DIHYDRO-2H-CHROMEN-4-YL]AMINO}PROPYL)ACETAMIDE

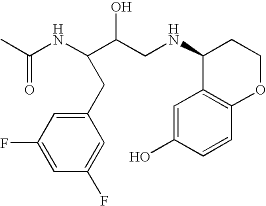

### Step 1: tert-butyl (1S,2R)-1-(3,5-difluorobenzyl)-2-hydroxy-3-{[(4S)-6-hydroxy-3,4-dihydro-2H-chromen-4-yl]amino}propylcarbamate

A mixture of (4S)-4-aminochromen-6-ol (165 mg, 1.0 mmol) and tert-butyl (1S)-2-(3,5-difluorophenyl)-1-[(2S)-oxiran-2-yl]ethylcarbamate (300 mg, 1.0 mmol) in 2-propanol (5 mL) was stirred at 60° C. for 16 h. The solvent was removed under reduced pressure. Flash chromatography (silica gel, 05% methanol/methylene chloride) recovered 54 mg of starting amine and provided the desired product (200 mg, 64%): ¹H NMR (300 MHz, CDCl₃) δ 7.26-6.63 (m, 6H), 4.55 (d, J=9.0 Hz, 1H), 4.21-4.14 (m, 2H), 3.73-3.71 (m, 2H), 3.47-3.44 (m, 1H), 3.10-3.02 (m, 1H), 2.84-2.75 (m, 3H), 2.10-2.02 (m, 1H), 1.94-1.90 (m, 1H), 1.37 (s, 9H).

### Step 2: (4S)-4-{[(2R,3S)-3-amino-4-(3,5-difluorophenyl)-2-hydroxybutyl]amino}chroman-6-ol hydrochloride

The above compound was prepared essentially according to the method of Example 72, step 7. ESI MS m/z 365 [C₁₉H₂₂F₂N₂O₃+H]⁺. This material was used in the next step without further purification.

### Step 3: N-((1S,2R)-1-(3,5-difluorobenzyl)-2-hydroxy-3-{[(4S)-6-hydroxy-3,4-dihydro-2H-chromen-4-yl]amino}propyl)acetamide

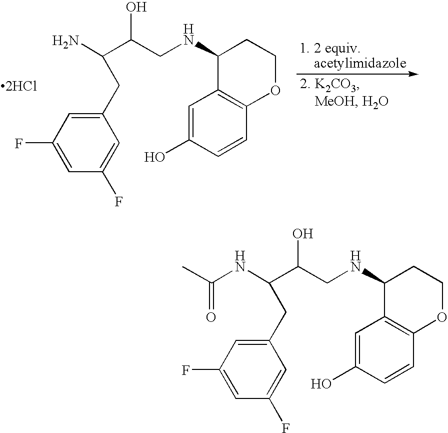

Triethylamine (217 mg, 2.15 mmol), followed by 1-acetylimidazole (95 mg, 0.86 mmol), was added to a solution of the product from step 2 (200 mg, 0.43 mmol) in methylene chloride (5 mL). The reaction mixture was stirred at room temperature for 16 h. The solvent was removed under reduced pressure. The residue was dissolved in methanol (6 mL) and water (3 mL) and treated with potassium carbonate (300 mg, 2.17 mmol). The reaction mixture was stirred at room temperature for 2 h. The solvent was removed under reduced pressure. The residue was acidified with 1N hydrochloric acid and extracted with ethyl acetate. The combined extracts were washed with saturated sodium chloride, and dried (sodium sulfate), filtered, and concentrated under reduced pressure. Purification by flash column chromatography (silica gel, 0-5% methanol/methylene chloride provided the desired product (85 mg, 49%) as a white foam. ESI MS m/z 407 [C₂₁H₂₄F₂N₂O₄+H]⁺; HPLC (Phenomenex Luna C18(2) Column, 150×4.6 mm, 5μ; A: 0.05% TFA in 95:5 H₂O/CH₃CN; B: 0.05% TFA in 5:95 H₂O/CH₃CN; Gradient: 30-100% B over 15 min; flow 1.0 mumin; Detection: 254 nm) 98.0% (AUC), t_{R}=7.01 min. Anal. Calc'd for C₂₁H₂₄F₂N₂O₄.0.25 H₂O: C, 61.38; H, 6.01; N, 6.82; found: C, 61.60; H, 5.68; N, 6.59.

### EXAMPLES 74-77

### GENERAL SCHEME FOR PREPARING ISOCHROMEN-4-YL COMPOUNDS

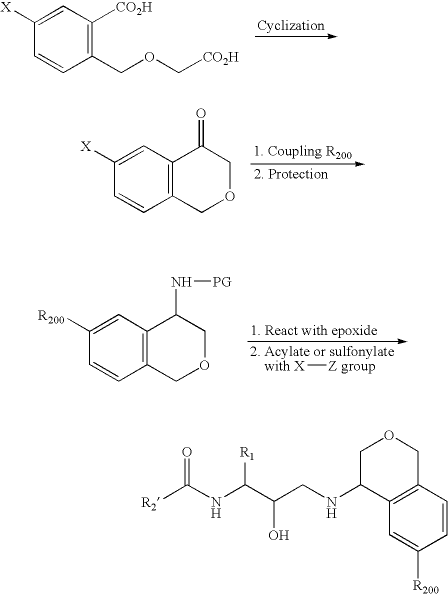

### EXAMPLE 74

### N-[(1S,2R)-1-(3,5-DIFLUOROBENZYL)-3-(3,4-DIHYDRO-1H-ISOCHROMEN-4-YLAMINO)-2-HYDROXYPROPYL]ACETAMIDE

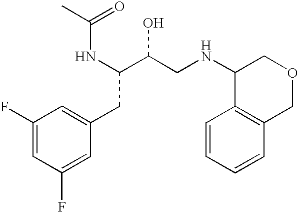

### Step 1: 2-[(Carboxymethoxy)methyl]benzoic acid

A mixture of (2-cyano-benzyloxy)-acetic acid ethyl ester (J. Org. Chem., 1985, 50, 2128) (30 g, 136 mmol) and KOH (38 g, 680 mmol) in 1:1 EtOH/H₂O (270 mL) was heated to 90° C. (oil bath) for 15 h. After cooling to room temperature the mixture was treated with concentrated HCl to a pH of 1 and extracted with CH₂Cl₂. The combined organic layers were dried concentrated in vacuo to yield an orange oil. The oil was dissolved in aq. Na₂CO₃, treated with activated carbon, filtered and the pH was adjusted to 1 with conc. HCl. The resulting solid was collected by filtration and dried to yield 8.2 g of 2-[(carboxymethoxy)methyl]benzoic acid as a tan solid. ¹H NMR (400 MHz, DMSO-d₆) δ 12.9 (bs, 1 H), 7.87 (dd, J=7.77, 1.14 Hz, 1 H), 7.66 (m, 1 H), 7.59 (m, 1 H), 7.39 (m, 1 H), 4.90 (s, 2 H), 4.15 (m, 2 H).

### Step 2: 1 H-Isochromen-4(3H)-one

A mixture of the product of, step 1 (8.2 g, 39.0 mmol), KOAc (16.5 g, 167.8 mmol) and Ac₂O (117 mL) was heated to reflux for 2 h. The mixture was cooled to room temperature then poured onto ice. The mixture was extracted with Et₂O, dried (MgSO₄), filtered, and concentrated in vacuo. The resulting residue was dissolved in 40 mL of EtOH, then 2 N NaOH (15 mL) was added. Stirring was continued at room temperature for 2 h then the EtOH was removed in vacuo. The resulting aqueous layer was extracted with Et₂O and the combined organic layers dried (magnesium sulfate), and concentrated in vacuo to yield 2.7 g of 1H-isochromen-4(3H)-one as a pale yellow oil after flash chromatography (10% EtOAc/Hexanes) R_{f}=0.25. ¹H NMR (400 MHz, CDCl₃) δ 8.05 (d, J=7.88 Hz, 1 H), 7.59 (m, 1 H), 7.43 (appt, J=7.36 Hz, 1 H), 7.24 (d, J=7.67 Hz, 1 H), 4.91 (s, 2 H), 4.39 (s, 2 H). Anal calc'd for C₉H₈O₂; C, 72.96; H, 5.44; found C, 72.50; H, 5.29. MS (ESI+) for C₉H₈O₂ m/z 148.8 [M+H]⁺.

### EXAMPLE 75

### ALTERNATIVE PREPARATION OF N-[(1S,2R)-1-(3,5-DIFLUOROBENZYL)-3-(3,4-DIHYDRO-1H-ISOCHROMEN-4-YLAMINO)-2-HYDROXYPROPYL]ACETAMIDE

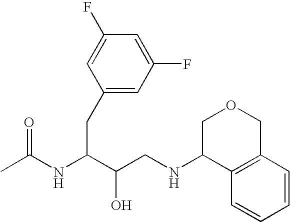

### Step 1: 1-[(Allyloxy)methyl]-2-iodobenzene

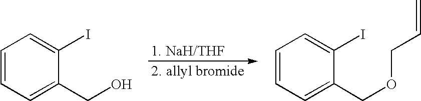

NaH (5.12 g, 128 mmol) was added to a THF (200 mL) solution of 2-iodo-benzyl alcohol (25 g, 107 mmol), at room temperature, in small portions. After complete addition of the NaH the allyl bromide (11.1 mL, 128 mmol) was added via syringe. The mixture was stirred overnight at room temperature. The resulting white heterogeneous mixture was quenched with H₂O (100 mL) and diluted with 300 mL of Et₂O. The organic layer was washed with H₂O and brine, dried (magnesium sulfate), filtered, and concentrated in vacuo to yield 1-[(allyloxy)methyl]-2-iodobenzene (31 g) as a faint yellow oil. HRMS (ESI+) calc'd for C₁₀H₁₁IO m/z 273.9857 [M+H]+; found 273.9855.

### Step 2: 1 H-Isochromen-4(3H)-one

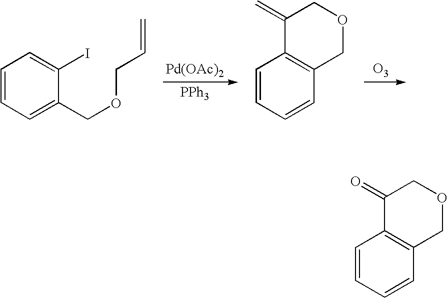

1-[(Allyloxy)methyl]-2-iodobenzene (23 g, 83.9 mmol) was dissolved in 100 mL of CH₃CN and 58.mL of Et₃N. The solution was vacuum degassed, then Pd(OAc)₂ (0.9 g, 4.2 mmol) and PPh₃ (2.2 g, 8.4 mmol) were added. The mixture was heated to 80° C. until HPLC indicated complete reaction. The mixture was cooled to room temperature and diluted with Et₂O (200 mL), washed with 1N HCl, NaHCO₃, and brine (1×50 mL), dried (sodium sulfate), filtered, and concentrated in vacuo to yield 4-methylene-3,4-dihydro-1H-isochromene (Heterocycles 1994, 39, 497) as an oil. HRMS (ESI+) calc'd for C₁₀H₁₀O m/z 146.0732 [M+H]⁺; found 146.0728. The crude oil was dissolved in 1:1 CH₃OH/CH₂Cl₂ (500 mL) and 5 mL of pyridine added. The mixture was chilled to −78° C. and ozone was bubbled through the mixture for 1 h. The mixture was purged with N₂(g) at −78 ° C. and treated with Me₂S, then allowed to warm to room temperature and stir for 3 h. The reaction was then diluted with CH₂Cl₂, washed with H₂O and brine, dried (sodium sulfate), filtered, and concentrated in vacuo to yield 1H-isochromen-4(3H)-one (5.1 g) as a pale yellow oil after flash chromatography (10% EtOAc/Hexanes) R_{f}=0.25. ¹H NMR (400 MHz, CDCl₃) δ 8.05 (d, J=7.88 Hz, 1 H), 7.59 (m, 1 H), 7.43 (appt, J=7.36 Hz, 1 H), 7.24 (d, J=7.67 Hz, 1 H), 4.91 (s, 2 H), 4.39 (s, 2 H. Anal calc'd for C₉H₈O₂; C, 72.96; H, 5.44; found C, 72.50; H, 5.29. MS (ESI+) for C₉H₈O₂ m/z 148.8 [M+H]⁺.

### Step 3: 3,4-dihydro-1H-isochromen4-ol

The alcohol was prepared from the ketone essentially according to the method of Example 50, step 1; it was obtained as a white solid. ¹H NMR (400 MHz, CDCl₃) δ 7.48 (m, 1 H), 7.31 (m, 2 H), 7.04 (m, 1 H), 4.84 (d, J=15 Hz, 1 H), 4.72 (d, J=15 Hz, 1 H), 4.58 (appt, J=2.38 Hz, 1 H), 4.14 (dd, J=12.02, 2.70 Hz, 1 H), 3.91 (dd, J=12.02, 2.70 Hz, 1 H), 2.24 (bs, 1 H). Anal calc'd for C₉H₁₀O₂; C, 71.98; H, 6.71; found C, 71.80; H, 6.94.

### Step 4: 3,4-Dihydro-1H-isochromen-4-amine

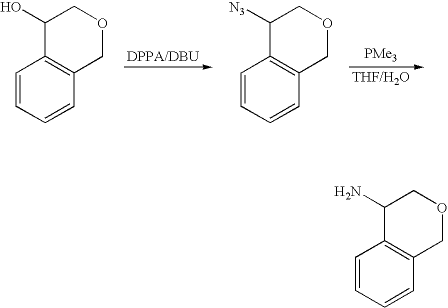

The above compound was prepared from the alcohol, essentially according to the method of Example 52, step 2. First, the alcohol was converted to the azide, which is obtained as a yellow oil. ¹H NMR (300 MHz, CDCl₃) δ 7.41-7.09 (m, 4 H), 4.90 (d, J=15.26 Hz, 1 H), 4.75 (d, J=15.26 Hz, 1 H), 4.23 (m, 2 H), 3.98 (dd, J=12.43, 3.39 Hz, 1 H). The crude azide was then reduced using PMe₃, yielding the amine. ¹H NMR (300 MHz, CDCl₃) δ 7.42 (m, 1 H), 7.30-7.22 (m, 2 H), 7.01 (m, 1 H), 4.85 (d, J=15 Hz, 1 H), 4.75 (d, J=15 Hz, 1 H), 4.00-3.86 (m, 3 H), 1.80 (bs, 2 H). ¹³C NMR (100 MHz, CDCl₃), δ 138.4, 134.6, 128.6, 127.5, 127.4, 124.5, 72.75, 68.61, 48.23.MS (ESI+) for C₉H₁₁NO m/z 133.2 [M+H]⁺ (loss of NH₂).

### Step 5: tert-butyl (1S,2R)-1-(3,5-difluorobenzyl)-3-(3,4-dihydro-1H-isochromen-4-ylamino)-2-hydroxypropylcarbamate

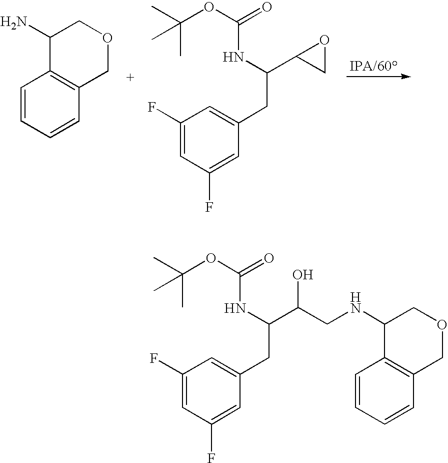

The coupled product was prepared essentially according to the method of Example 50, step 3; the resulting mixture of epimers was obtained as an off white solid and was used in the next step without further purification. HRMS (ESI+) calc'd for C₂₄H₃₀F₂N₂O₄ m/z 449.2252 [M+H]⁺; found 449.2244.

### Step 6: N-[(1S,2R)-1-(3,5-Difluorobenzyl)-3-(3,4-dihydro-1H-isochromen-4-ylamino)-2-hydroxypropyl]acetamide

The above compound was prepared essentially according to the method of Example 3, steps 7-8 and Example 50, steps 3-4; the acetamide was obtained as a white foam. Small scale reverse phase HPLC of the mixture of epimers results in partial separation.

¹H NMR (400 MHz, CDCl₃) δ 7.35 (m, 1 H), 7.28 (m, 2 H), 7.04 (m, 1 H), 6.77 (m, 2 H), 6.68 (m, 1 H), 5.90 (d, J=8.50 Hz, 1 H), 4.83 (d, J=15.13 Hz, 1 H), 4.73 (d, J=15.13 Hz, 1 H), 4.18 (m, 2 H), 3.85 (dd, J=11.82, 2.90 Hz, 1 H), 3.70 (m, 1 H), 3.63 (m, 1 H), 3.00-2.84 (m, 3 H), 2.71 (dd, J=12.34, 7.15 Hz, 1 H), 1.93 (s, 3 H). MS (ESI+) for C₂₁H₂₄F₂N₂O₃ m/z 391.5 [M+H]⁺. ¹H NMR (400 MHz, CDCl₃) δ 7.40 (m, 1 H), 4.87 (d, J=2 H), 7.05 (m, 1 H), 6.77 (m, 2 H), 6.68 (m, 1 H), 5.88 (d, J=8.91 Hz, 1 H), 4.87 (d, J=15.13 Hz, 1 H), 4.74 (d, J=15.13 Hz, 1 H), 4.26-4.16 (m, 2 H), 3.84 (m,2 H), 3.75 (bs, 1 H), 3.57 (m, 2 H), 3.04-2.85 (m, 3 H), 2.76 (dd, J=12.34, 6.53 Hz, 1 H), 1.90 (s, 3 H). MS (ESI+) for C₂₁H₂₄F₂N₂O₃ m/z 391.5 [M+H]⁺.

### EXAMPLE 76

### PREPARATION OF N-{(1S,2R)-1-(3,5-DIFLUOROBENZYL)-2-HYDROXY-3-[(6-ISOPROPOXY-1,1-DIMETHYL-3,4-DIHYDRO-1H-ISOCHROMEN-4-YL)AMINO]PROPYL}ACETAMIDE

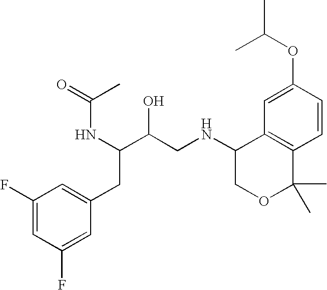

### Step 1: 6-Isopropoxy-1,1-dimethyl-3,4-dihydro-1H-isochromene

The ether was prepared from the alcohol essentially according to the method of Example 72, step 3; the ether was obtained as a pale yellow oil: ¹H NMR (300 MHz, CDCl₃) δ 7.00 (d, J=8.5 Hz, 1H), 6.71 (dd, J=8.5, 2.6 Hz, 1H), 6.59 (d, J=2.5 Hz, 1H), 4.54-4.46 (m, 1H), 3.92 (t, J=5.5 Hz, 2H), 2.77 (t, J=5.5 Hz, 2H), 1.49 (s, 6H), 1.32 (d, J=6.0 Hz, 6H).

### Step 2: 4-Bromo-6-isopropoxy-1,1-dimethyl-3,4-dihydro-1 H-isochromene

A solution of the product from step 1 (0.22 g, 1.0 mmol), N-bromosuccinimide (0.19 g, 1.05 mmol), and AIBN (catalytic) in carbon tetrachloride (3 mL) was degassed with N₂(g) for 10 min, and then stirred at 65° C. for 2.5 h. The reaction mixture was cooled in an ice-water bath, diluted with methylene chloride (150 mL), washed with water, saturated sodium chloride, dried (sodium sulfate), filtered, and concentrated. The crude product was purified by flash chromatography (silica, 10:1 hexanes/ethyl acetate) to yield the bromide (1.02 g, 53%) as a pale-yellow oil: ¹H NMR (300 MHz, CDCl₃) δ 6.98 (d, J=8.5 Hz, 1H), 6.86 (d, J=2.5 Hz, 1H), 6.80 (dd, J=8.5, 2.6 Hz, 1H), 5.18 (m, 1H), 4.54-4.48 (m, 1H), 4.19 (dd, J=12.8, 3.0 Hz, 1H), 4.11 (dd, J=12.8, 3.0 Hz,1 H), 1.59 (s, 3H), 1.47 (s, 3H), 1.33 (d, J=6.0 Hz, 6H).

### Step 3: tert-Butyl (1S,2R)-1-(3,5-difluorobenzyl)-2-hydroxy-3-[(6-isopropoxy-1,1-dimethyl-3,4-dihydro-1H-isochromen-4-yl)amino]propylcarbamate

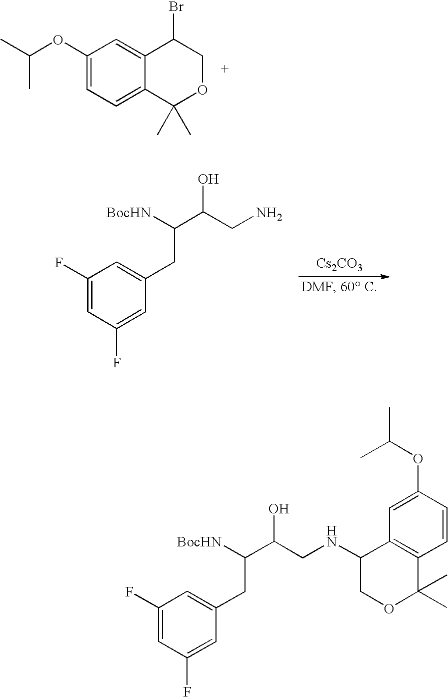

A solution of 4-bromo-6-isopropoxy-1,1-dimethyl-3,4-dihydro-1H-isochromene (0.61 g, 2.04 mmol), cesium carbonate (1.33 g, 4.08 mmol), and tert-butyl (1S,2R)-3-amino-1-(3,5-difluorobenzyl)-2-hydroxypropylcarbamate (0.64 g, 2.04 mmol) in N,N-dimethylformamide (10 mL) was stirred at 60° C., under N₂(g), for 24 h. The reaction mixture was diluted with ethyl acetate (100 mL) and washed with 5% lithium chloride, water, saturated sodium chloride (30 mL), dried (sodium sulfate), and concentrated under reduced pressure. The crude product was purified by flash chromatography (silica, 95:5 methylene chloride/methanol) to yield the desired product (0.51 g, 47%) as a pale-yellow foam: ESI MS m/z 535 [C₂₉H₄₀F₂N₂O₅ +H]⁺.

### Step 4: (2R,3S)-3-Amino-4-(3,5-difluorophenyl)-1-[(6-isopropoxy-1,1-dimethyl-3,4-dihydro-1H-isochromen-4-yl)amino]butan-2-ol hydrochloride

The free amine was prepared from the Boc-amine essentially according to the method of Example 72, step 6; the amine was obtained as a yellow solid: ESI MS m/z 435 [C₂₄H₃₂F₂N₂O₃+H]⁺.

### Step 5: N-{(1S,2R)-1-(3,5-Difluorobenzyl)-2-hydroxy-3-[(6-isopropoxy-1,1-dimethyl-3,4-dihydro-1H-isochromen-4-yl)amino]propyl}acetamide

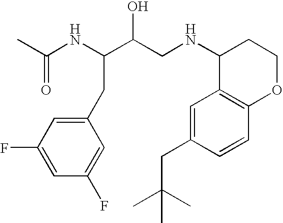

The acetamide was prepared from the free amine essentially according to the method of Example 72, step 7. The crude product was purified by flash chromatography (silica, 95:5 methylene chloride/methanol) to yield the acetamide as a white foam: ¹H NMR (300 MHz, CDCl₃) δ 7.01 (d, J=8.4 Hz, 1H), 6.82-6.74 (m, 4H), 6.69-6.63 (m, 1H), 5.81-5.78 (m, 1H), 4.564.52 (m, 1H), 4.21-4.17 (m, 1H), 3.94 (d, J=2.1 Hz, 2H), 3.50-3.48 (m, 2H), 3.00-2.85 (m, 3H), 2.71-2.64 (m, 1 H), 1.88 (s, 3H), 1.52 (s, 3H), 1.45 (s, 3H), 1.33 (d, J=6.0 Hz, 6H); ESI MS m/z 477 [C₂₆H₃₄F₂N₂O₄+H]⁺; HPLC (Phenomenex Luna C18(2) Column, 150×4.6 mm, 5μ; A: 0.05% TFA in 95:5 H₂O/CH₃CN; B: 0.05% TFA in 5:95 H₂O/CH₃CN; Gradient: 10-90% B over 15 min; flow 1.0 mL/min; Detection: 254 nm) >99% mixture of diastereomers (AUC), t_{R}=6.12 and 6.77 min.

### EXAMPLE 77

### PREPARATION OF N-{(1S,2R)-1-(3,5-DIFLUOROBENZYL)-2-HYDROXY-3-[(6-NEOPENTYL-3,4-DIHYDRO-1H-ISOCHROMEN-4-YL)AMINO]PROPYL}ACETAMIDE

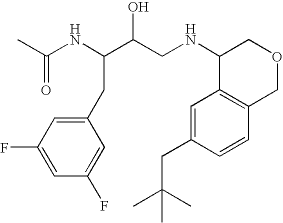

### Step 1: 5-Bromo-2-carboxymethoxymethyl-benzoic acid

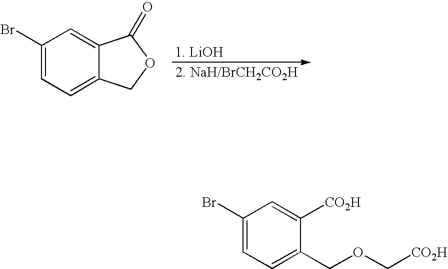

Lithium hydroxide monohydrate (11.80 g, 281.6 mmol) was added at room temperature over several minutes to a solution of 5-bromophthalide (20.0 g, 93.88 mmol) in a 2:1:1 solution of tetrahydrofuran/methanol/water (570 mL) and the reaction mixture stirred at room temperature overnight. The reaction mixture was concentrated under reduced pressure and azeotropically dried with benzene to give 5-bromo-2-hydroxymethyl-benzoic acid as a white solid. The material was used without further purification: ¹H NMR (300 MHz, CDCl₃+CD₃OD) δ 7.89 (d, J=8.3 Hz, 1H), 7.67 (d, J=1.9 Hz, 1H), 7.50 (dd, J=8.3, 1.9 Hz, 1H), 3.99 (s, 2H); MS (ESI−) m/z 229 [C₈H₇BrO₃−H]⁻. Sodium hydride (15.0 g, 375 mmol, 60% dispersion in mineral oil) was added in small portions over the course of 0.5 h at room temperature to a solution of 5-bromo-2-hydroxymethyl-benzoic acid in tetrahydrofuran (235 mL) containing bromoacetic acid (14.35 g, 103.2 mmol) and sodium iodide (1.41 g, 9.4 mmol). The reaction mixture was heated at reflux overnight. The reaction mixture was cooled to room temperature and poured into water and then extracted with diethyl ether. The aqueous phase was acidified with 10% hydrochloric acid to pH 3-4 and extracted several times with ethyl acetate. The combined ethyl acetate phases were washed with water and saturated sodium chloride, dried (sodium sulfate), filtered, and concentrated to yield 5-bromo-2-carboxymethoxymethyl-benzoic acid as a white solid. The material was used without further purification: ¹H NMR (300 MHz, CD₃OD) δ 7.93-7.86 (m, 2H), 7.55-7.50 (m, 1H), 4.98 (s, 2H), 4.23 (s, 2H); MS (ESI−) m/z 287 [C₁₀H₉BrO₅−H]⁻.

### Step 2: 6-Bromo-isochroman-4-one

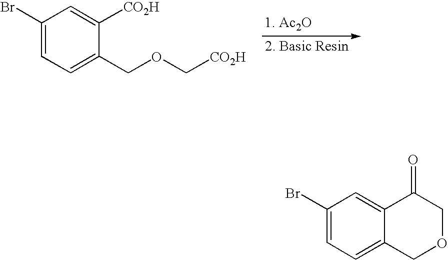

A solution of bromo-2-carboxymethoxymethyl-benzoic acid in acetic anhydride (350 mL) containing potassium acetate (170 g) was heated at reflux for 2 h. The reaction mixture was cooled to room temperature and concentrated under reduced pressure and the residue partitioned between ethyl acetate and water. The phases were separated and the aqueous phase extracted with ethyl acetate. The combined organic phase was then washed with saturated sodium chloride, dried (sodium sulfate), filtered, and concentrated to yield a red semi-solid. Purification by flash column chromatography over silica (85:15 hexanes/ethyl acetate) yielded the enol acetate (7.59 g, 29% for three steps) as a golden syrup: ¹H NMR (300 MHz, CDCl₃) δ 7.37 (dd, J=8.2, 1.9 Hz, 1H), 7.19 (d, J=1.9 Hz, 1H), 6.82 (d, J=8.2 Hz, 1H), 5.04 (s, 2H), 2.29 (s, 3H). Unactivated Dowex 500A OH anion exchange resin (1 g) added in one portion to a solution of the acetate enol acetate (5.95 g, 22.11 mmol) in methanol (50 mL) and the reaction mixture stirred at room temperature overnight. The reaction mixture was gravity filtered and the resin washed with fresh methanol. The combined filtrate was then concentrated under reduced pressure to yield 6-bromo-isochromen-4-one (4.32 g, 86%) as a yellow oil, which solidified on standing: ¹H NMR (300 MHz, CDCl₃) δ 7.90 (d, J=8.3 Hz, 1H), 7.56 (dd, J=8.3, 1.7 Hz, 1H), 7.41 (d, J=1.7 Hz, 1H), 4.86 (s, 2H), 4.36 (s, 2H).

### Step 3: 6-Bromo-isochroman-4-ol

A solution of sodium borohydride (300 mg, 7.93 mmol) in a minimum amount of ice cold water was added dropwise at 0° C. to a solution of 6-bromo-isochromen-4-one (1.49 g, 6.56 mmol) in absolute ethanol (27.0 mL). The reaction mixture was stirred at room temperature for 2 h. The reaction mixture was partitioned between ethyl acetate and saturated sodium bicarbonate solution. The phases were separated and the organic phase was washed with water and saturated sodium chloride, dried (sodium sulfate), filtered, and concentrated under reduced pressure to yield 6-Bromo-isochromen-4-ol (1.44 g, 95%) as a white solid: ¹H NMR (300 MHz, CDCl₃) δ 7.40 (dd, J=8.3, 1.8 Hz, 1H), 7.30 (d, J=8.3 Hz, 1H), 7.15 (d, J=1.8 Hz, 1H), 4.63 (ABq, J=15.3 Hz, 2H), 4.49 (d, J=8.6 Hz, 1H), 4.07 (dd, J=12.0, 2.8 Hz, 1H), 3.83 (dd, J=12.0, 2.8 Hz, 1H), 2.60 (d, J=9.2 Hz, 1H).

### Step 4: (6-Bromo-isochroman-4-yl)-carbamic acid tert-butyl ester

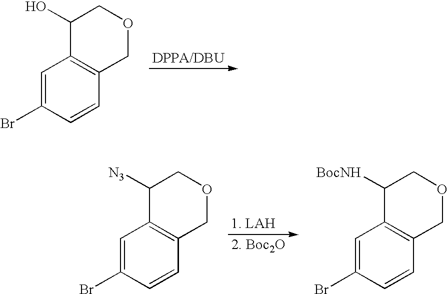

Diphenyphosphoryl azide (2.11 mL, 9.8 mmol) was added at 0° C. to a solution of 6-bromo-isochromen-4-ol (1.87 g, 8.16 mmol) in toluene (17 mL). A mixture, of 1,8-diazabicyclo[5.4.0]undec-7-ene (1.46 mL, 9.8 mmol) in toluene (5.0 mL) was added dropwise to this over 0.5 h. The reaction mixture was then stirred at room temperature overnight. The reaction mixture was then passed through a plug of silica and the plug was rinsed with 6:1 hexanes/ethyl acetate. The combined filtrates were concentrated under reduced pressure to provide the azide as a yellow oil: ¹H NMR (300 MHz, CDCl₃) δ 7.46-7.33 (m, 3H), 4.76 (ABq, J=15.5 Hz, 2H), 4.22-4.16 (m, 3H), 3.93 (dd, J=11.7, 2.6 Hz, 1H). A solution of lithium aluminum hydride (391 mg, 9.79 mmol) in a minimum amount of tetrahydrofuran (2.0 mL) was added dropwise at 0° C. to a solution of the azide in tetrahydrofuran (30 mL). The reaction mixture was heated at reflux for 1 h. The reaction mixture was cooled to room temperature and quenched with water (0.5 mL), 15% sodium hydroxide. (1.2 mL), and water (0.5 mL), then stirred at room temperature for 1 h. The resulting mixture was then passed through a plug of silica and the plug was rinsed with ether. The combined filtrates were concentrated under reduced pressure to yield an oil, which was dissolved in a minimum amount of ethyl acetate. Hydrogen chloride (3.0 mL, 4 N in 1,4-dioxane, 12 mmol) was added and the reaction was stirred at room temperature overnight. The reaction mixture was vacuum filtered to yield the desired amine salt (1.54 g, 72 % for 2 steps) as a white solid: ¹H NMR (300 MHz, CDCl₃) δ 7.54-7.44 (m, 2H), 7.37 (s, 1H), 4.80 (ABq, J=15.5 Hz, 2H), 4.42 (d, J=12.8 Hz, 1H), 4.34 (s, 1H), 3.87 (dd, J=12.8, 2.2 Hz, 1H), 3.66 (s, 3 H); ESI MS m/z 228 [C₉H₁₀BrNO+H]⁺.

Di-tert-butyl dicarbonate (1.40 g, 6.40 mmol) was added in portions to a solution of amine (1.54 g, 5.82 mmol) in acetonitrile (25 mL) containing N,N-diisopropylethylamine (4.0 mL, 23.28 mmol). The reaction was stirred at room temperature overnight, concentrated under reduced pressure and partitioned between ethyl acetate and water. The organic phase was dried (sodium sulfate), filtered, and concentrated under reduced pressure to yield a yellow syrup. Purification by flash column chromatography over silica (80:20 hexanes/ethyl acetate) yielded the desired product (1.05 g, 55%) as a white solid: ¹H NMR (300 MHz, CDCl₃) δ 7.41-7.23 (m, 2H), 7.15 (s, 1H), 5.10-5.07 (m, 1H), 4.69 (ABq, J=15.5 Hz, 2H), 4.04-4.00 (m, 1H), 3.89-3.81 (m, 1 H), 1.45 (s, 9 H).

### Step 5: 6-(2,2-Dimethyl-propyl)-isochroman-4-ylamine hydrochloride

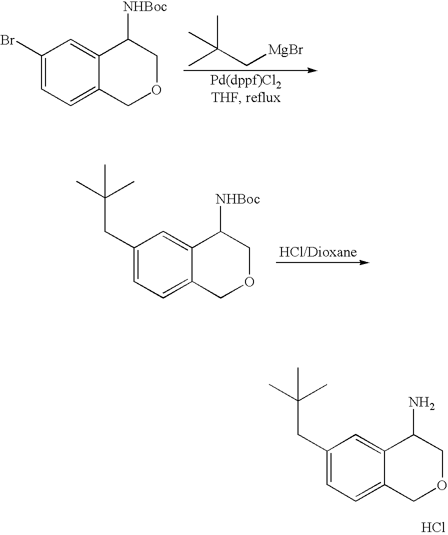

Neopentylmagnesium bromide (10 mL, 9.1 mmol, 1.0 M in ether) was added dropwise to a solution of zinc chloride (18.2 mL, 0.5 M in tetrahydrofuran, 9.1 mmol) over 0.5 h and the reaction mixture stirred at room temperature for an additional 0.5 h. [1,1′-Bis(diphenylphosphino)ferrocene]dichloropalladium(II) complex with dichloromethane (1:1) (250 mg, 0.30 mmol) was added to the reaction mixture followed by (6-bromo-isochromen-4-yl)-carbamic acid tert-butyl ester (1.00 g, 3.04 mmol) and the reaction mixture heated at reflux for 1 h. The reaction mixture was cooled and then concentrated under reduced pressure. The residue was re-dissolved in ethyl acetate and washed with water, sodium chloride, dried (sodium sulfate), filtered, and concentrated under reduced pressure. Purification by flash column chromatography over silica (83:17 hexanes/ethyl acetate) yielded the desired protected amine (303 mg, 31%) as a white solid: ¹H NMR (300 MHz, CDCl₃) δ 7.30-7.23 (m, 1H), 7.00 (d, J=6.3 Hz, 1H), 6.74 (s, 1H), 5.09-5.06 (m, 1H), 4.79-4.65 (m, 3H), 4.13-3.85 (m, 2H), 2.45 (s, 2H), 1.46 (s, 9 H), 0.89 (s, 9H); ESI MS m/z 320 [C₁₉H₂₉NO₃+H]⁺. A solution of protected amine (303 mg, 0.95 mmol) and hydrogen chloride (20 mL, 4 N in 1,4-dioxane, 80 mmol) was stirred at room temperature overnight. The reaction mixture was concentrated under reduced pressure to give 6-(2,2-dimethyl-propyl)-isochromen-4-ylamine hydrochloride (210 mg, quantitative) as a white solid: ¹H NMR (300 MHz, CDCl₃) δ 7.28 (d, J=7.6 Hz, 1H), 7.01 (d, J=7.6, 1.2 Hz, 1H), 6.73 (d, J=1.2 Hz, 1H), 4.75 (ABq, J=15.0 Hz, 2H), 3.96-3.80 (m, 3H), 2.44 (s, 2H), 1.73 (m, 2H), 0.89 (s, 9H); ESI MS m/z 220 [C₁₄H₂₁NO+H]⁺.

### Step 6: tert-Butyl (1S,2R)-1-(3,5-difluorobenzyl)-2-hydroxy-3-[(6-neopentyl-3,4-dihydro-1H-isochromen-4-yl)amino]propylcarbamate

The above compound was prepared essentially according to the method of Example 50, step 3. The resulting crude material was purified by flash column chromatography over silica (94:6 chloroform/methanol) to yield the desired product as a white foam: ESI MS m/z 519 [C₂₉H₄₀F₂N₂O₄+H]⁺.

### Step 7: N-{(1S,2R)-1-(3,5-Difluorobenzyl)-2-hydroxy-3-[(6-neopentyl-3,4-dihydro-1 H-isochromen-4-yl)amino]propyl}acetamide

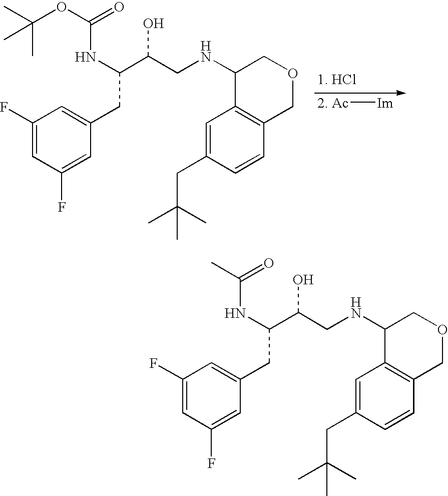

The acetamide was prepared from the Boc-protected amine essentially according to the methods described above, for example see Example 3, steps 7-8, and Example 72, steps 6-7. First, the Boc-protected amine was deprotected to yield the free amine as a white solid. Second, the free amine was acylated to form the acetamide, as a mixture of epimers. ¹H NMR (300 MHz, CDCl₃) δ 7.24-7.16 (m, 2H), 7.01-6.98 (m, 1H), 6.76-6.66 (m, 4H), 5.83 (ABq, J=15.0 Hz, 2H), 4.10-4.05 (m, 2H), 3.83-3.79 (m, 1H), 3.55-3.51 (m, 2H), 2.93-2.72 (m, 3H), 2.69-2.65 (m, 1H), 2.45 (s, 2H), 1.89 (m, 4H), 0.89 (s, 9H); ESI MS m/z 461 [C₂₆H₃₄F₂N₂O₃+H]⁺; HPLC (1-99, 220) 68.1% Major Epimer (AUC), t_{R}=10.89 min and 31.8% Minor Epimer (AUC), t_{R}=11.19 min.

### EXAMPLE 78

### PREPARATION OF N-((1S,2R)-1-[3-(ALLYLOXY)-5-FLUOROBENZYL]-3-{[(4R)-6-ETHYL-2,2-DIOXIDO-3,4-DIHYDRO-1H-ISOTHIOCHROMEN-4-YL]AMINO}-2-HYDROXYPROPYL)ACETAMIDE

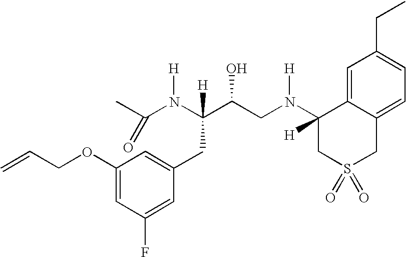

Using methods analogous to those previously described, tert-butyl (1S)-2-[3-(allyloxy)-5-fluorophenyl]-1-[(2S)-oxiran-2-yl]ethylcarbamate (0.37 mmol) and (4R)-6-ethyl-3,4-dihydro-1H-isothiochromen-4-amine 2,2-dioxide (0.78 mmol) were reacted together, and the product was further converted, using methods analogous to those previously described (except that the HCl salt is not formed), to N-((1S,2R)-1-[3-(allyloxy)-5-fluorobenzyl]-3-{[(4R)-6-ethyl-2,2-dioxido-3,4-dihydro-1 H-isothiochromen-4-yl]amino}-2-hydroxypropyl)acetamide (0.16 mmol, 43%), which was obtained as a white solid: ¹H NMR (CDCl₃) δ 7.22-7.19 (m, 2 H), 7.13 (m, 1 H), 6.57 (m, 1 H), 6.51 (m, 2 H), 6.06-5.99 (m, 1 H), 5.75 (br, 1 H), 5.41 (d, J=17 Hz, 1 H), 5.30 (d, J=12 Hz, 1 H), 4.67 (d, J=15 Hz, 1 H), 4.50 (m, 2 H), 4.26 (m, 1 H), 4.17 (d, J=15 Hz, 1 H), 4.1 (m, 1 H), 3.66 (m, 2 H), 3.48 (m, 1 H), 3.36 (dd, 1 H), 2.90 (m, 2 H), 2.78 (m, 2 H), 2.67 (q, J=7.6 Hz, 2 H), 1.91 (s, 3 H), 1.25 (t, J=7.6 Hz, 3 H); MS (Cl) m/z 505.4 [M+H]⁺.

### EXAMPLE 79

### SYNTHESIS OF N-[1-(3,5-DIFLUORO-BENZYL)-3-(6-ETHYL-3-HYDROXY-CHROMAN-4-YLAMINO)-2-HYDROXY-PROPYL]-ACETAMIDE PRECURSORS

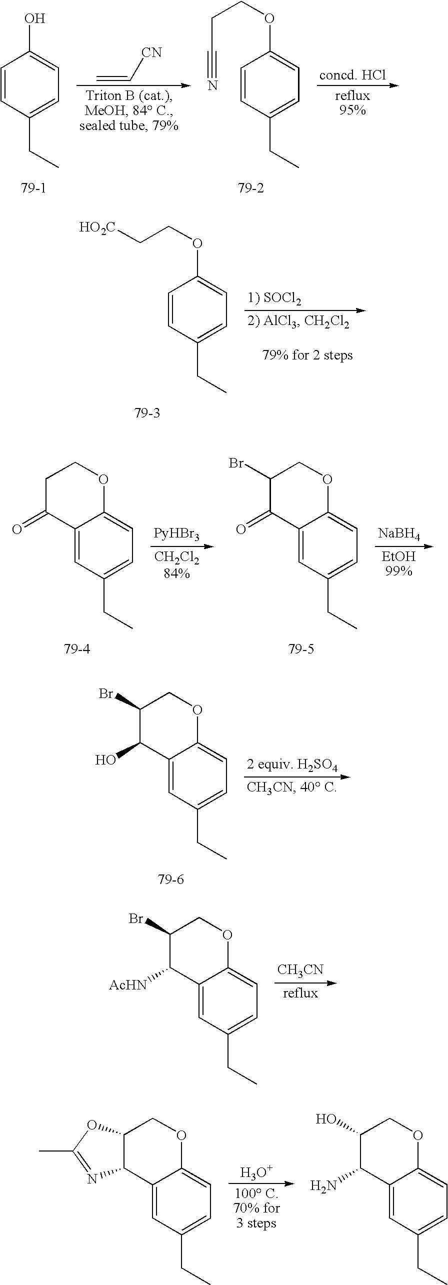

The synthesis of 4-amino-6-ethyl-chroman-3-ol is illustrated in the above scheme. In the above scheme, phenol 79-1 underwent Michael addition with acrylonitrile to give nitrile 79-2. Subsequent acid hydrolysis yielded carboxylic acid 79-3, which was then converted to the acid chloride and cyclized intramolecularly to give chromonone 79-4. Alpha bromination of ketone 79-4 yielded bromide 79-5, which was reduced with sodium borohydride to give bromo alcohol 79-6. Using Ritters reaction conditions, 79-6 was transformed to racemic 4-Amino-6-ethyl-chroman-3-ol. More specific experimental procedures follow the scheme.

### Step 1

A mixture of 4-ethylphenol 79-5 (26.69 g, 0.218 mol), acrylonitrile (50 mL, 0.754 mol, 3.5 equiv), and triton B (40 wt % in methanol, 5 mL, 0.011 mol, 0.05 equiv) was stirred at 84° C. in a sealed tube overnight. The reaction mixture was diluted with ether (300 mL) and the brown precipitate was removed by suction filtration. The ether solution was washed with 2 M sodium hydroxide aqueous-solution (2×100 mL), 1 M hydrochloric acid (100 mL) and saturated sodium chloride, dried (magnesium sulfate), and concentrated under reduced pressure. Purification by flash column chromatography (silica, gradient 10:1, and 6:1 hexanes/ethyl acetate) provided nitrile 79-6 (30.17 g, 79%) as a white solid: ¹H NMR (300 MHz, CDCl₃) δ 7.17-7.08 (m, 2H), 6.87-6.79 (m, 2H), 4.18 (t, J=6.4 Hz, 2H), 2.80 (t, J=6.4 Hz, 2H), 2.60 (q, J=7.6 Hz, 2H), 1.20 (t, J=7.6 Hz, 3H); ESI MS m/z 176 [C₁₁H₁₃NO+H]⁺.

### Step 2

Nitrile 35 (30.17 g, 0.172 mol) was stirred with a concentrated hydrochloric acid solution (100 mL, 1.20 mol, 7 equiv) at reflux overnight. White precipitate formed as the reaction proceeded. The reaction mixture was cooled to room temperature and the solid was collected by suction filtration. The filter cake was washed several times with cold water and dried in a vacuum oven at 50° C. for 14 h. The carboxylic acid was obtained as a white-solid (31.79 g, 95%): ¹H NMR (300 MHz, CDCl₃) δ 7.13-7.08 (m,2H), 6.88-6.80 (m, 2H), 4.20 (t, J=6.3 Hz, 2H), 2.85 (t, J=6.3 Hz, 2H), 2.58 (q, J-7.6 Hz, 2H), 1.18 (t, J=7.6 Hz, 3H); ESI MS m/z 193 [C₁₁H₁₄O₃−H].

### Step 3

The carboxylic acid (0.800 g, 4.12 mmol) was stirred with thionyl chloride (6 mL, 82.4 mmol, 20 equiv) at reflux for 2 h. Excess thionyl chloride was removed under reduced pressure. The acid chloride thus obtained was used without further purification in the next reaction.

Aluminum chloride (1.10 g, 8.24 mmol, 2 equiv) was added in one portion to a solution of acid chloride as above in dry methylene chloride (50 mL) and the resulting brown mixture was stirred at reflux for 14 h and cooled to room temperature. The mixture was poured onto crushed ice, then 6 M hydrochloric acid (20 mL) was added and the mixture was extracted with methylene chloride. The combined organics were washed with saturated sodium chloride, dried (magnesium sulfate), and concentrated under reduced pressure. Purification by flash column chromatography (silica, gradient 10:1, and 6:1 hexanes/ethyl acetate) yielded chromonone 37 (574 mg, 79%) as a colorless oil: ¹H NMR (300 MHz, CDCl₃) δ 7.72 (d, J=2.2 Hz, 1H), 7.32 (dd, J=8.5, 2.2 Hz, 1H), 6.90 (d, J=8.5 Hz, 1H), 4.52 (t, J=6.5 Hz, 2H), 2,80 (t, J=6.5 Hz, 2H), 2.60 (q, J=7.6 Hz, 2H), 1.20 (t, J=7.6 Hz, 3H); ESI MS m/z 177 [C₁₁H₁₂O₂+H]⁺.

### Step 4

Pyridinium hydrobromide perbromide (743 mg, 2.32 mmol) was added to a solution of the chromonone (372 mg, 2.11 mmol) in dry methylene chloride (15 mL) and the reaction mixture was stirred at room temperature for 2 h. Water (15 mL) was added to the mixture and the layers were separated. The aqueous layer was further extracted with methylene chloride. The combined organics were dried (magnesium sulfate) and concentrated under reduced pressure. Purification by flash column chromatography (silica, gradient 20:1, and 10:1 hexanes/ethyl acetate) provided the bromo ketone (450 mg, 84%) as a slightly yellow oil: ¹H NMR (300 MHz, CDCl₃) δ 7.77 (d, J=2.2 Hz, 1H), 7.39 (dd, J=8.5, 2.2 Hz, 1H), 6.97 (d, J=8.5 Hz, 1H), 4.68-4.52 (m, 3H), 2.62 (q, J=7.6 Hz, 2H), 1.22 (t, J=7.6 Hz, 3H); ESI MS m/z 255 [C₁₁C₁₁BrO₂+H]⁺.

### Step 5

Sodium borohydride (99 mg, 2.61 mmol, equiv) was added to a solution of the bromo ketone (444 mg, 1.74 mmol) in absolute ethanol (15 mL) and the reaction mixture was stirred at room temperature for 2 h. The reaction mixture was quenched by the addition of 1 M hydrochloric acid (4 mL) and most of ethanol was removed by rotary evaporation. The residue was partitioned between water and methylene chloride. The aqueous layer was further extracted with methylene chloride. The combined organics were dried (sodium sulfate) and concentrated under reduced pressure. The bromo alcohol was obtained as a white solid (443 mg, 99%) and used in the next step without further purification: ¹H NMR (300 MHz, CD₃OD) δ 7.14 (d, J=1.5 Hz, 1H), 7.03 (dd, J=8.3, 1.5 Hz, 1H), 6.69 (d, J=8.3 Hz, 1H), 4.78 (d, J=3.2 Hz, 1H), 4.58-4.49 (m, 1H), 4.35-4.26 (m,2H), 2.56 (q, J=7.6 Hz, 2H), 1.16 (t, J=7.6 Hz, 2H), 1.16 (t, J=7.6 Hz, 3H).

### Step 6

The bromo alcohol from step 5 (443 mg, 1.72 mmol) was dissolved in anhydrous acetonitrile (10 mL) and concentrated sulfuric acid (0.19 mL, 3.47 mmol) was added via syringe. The reaction mixture was stirred at 40° C. for 5 h and then reflux for 12 h. Water (10 mL) was added and most of the acetonitrile was removed under reduced pressure. 6 M hydrochloric acid (10 mL) was added to the residue and the resulting mixture was stirred at reflux for 14 h. The reaction mixture was cooled to room temperature, and placed in an ice bath. 6 M sodium hydroxide was added until pH 12, and the mixture was extracted with methylene chloride (3×50 mL). The combined organics were washed with saturated sodium chloride, dried (sodium sulfate) and concentrated. Purification by flash column chromatography (silica, gradient 20:1, 10:1 and 1:1 methylene chloride/methanol) provided 4-Amino-6-ethyl-chromen-3-ol (233 mg, 70%) as a white solid: ¹H NMR (300 MHz, CDCl₃) δ 7.12 (d, J=1.5 Hz, 1H), 7.01 (dd, J=8.3, 1.5 Hz, 1H) 6.78 (d, J=8.3 Hz, 1H), 4.09 (d, J=11.5 Hz, 1H), 4.00-3.91 (m, 2H), 3.88-3.75 (m, 1H), 2.58 (q, J=7.6 Hz, 2H), 1.60 (br s, 3H) 1.20 (t, J=7.6 Hz, 3H); ESI MS m/z 194 [C₁₁H₁₅NO₂+H]⁺; HPLC (Phenomenex Luna C18(2) Column, 150×4.6 mm, 4μ; A: 95:5 H₂O/CH₃CN; B: 5:95 H₂O/CH₃CN; Gradient: 1-99% B over 15 min; flow 1.0 mL/min; Detection: 254 nm) 96.7% (AUC), t_{R}=9.4 min.

### EXAMPLE 80

### SYNTHESIS OF N-[1-(3,5-DIFLUORO-BENZYL)-3-(6-ETHYL-3-HYDROXY-CHROMEN-4-YLAMINO)-2-HYDROXY-PROPYL]-ACETAMIDE

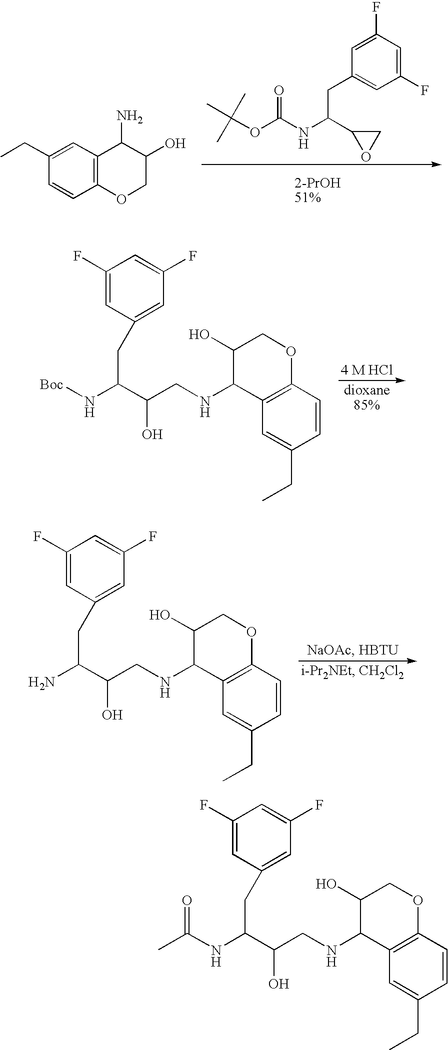

Coupling of racemic 4-amino-6-ethyl-chromen-3-ol with tert-butyl (1S)-2-(3,5-difluorophenyl)-1-[(2S)-oxiran-2-yl]ethylcarbamate, followed by Boc deprotection and HBTU-mediated acylation yielded N-[1-(3,5-difluoro-benzyl)-3-(6-ethyl-3-hydroxy-chromen-4-ylamino)-2-hydroxy-propyl]-acetamide, as a mixture of diastereomers. One possible procedure for preparing N-[1-(3,5-difluoro-benzyl)-3-(6-ethyl-3-hydroxy-chromen-4-ylamino)-2-hydroxy-propyl]-acetamide is described below.

### Step 1

Tert-butyl (1S)-2-(3,5-difluorophenyl)-1-[(2S)-oxiran-2-yl]ethylcarbamate (1.40 g, 4.71 mmol) was added to a solution of 4-amino-6-ethyl-chromen-3-ol (1.00 g, 5.18 mmol) in 2-propanol (60 mL) and the reaction mixture was heated at 50° C. for 17 h and then at 80° C. for 1 h. The reaction mixture was cooled to room temperature and the solvent was removed under reduced pressure. The residue was partitioned between methylene chloride (20 mL) and water (20 mL). The aqueous phase was extracted with methylene chloride, and the combined organic phase was washed successively with 0.5 N hydrochloric acid (10 mL), saturated sodium bicarbonate (10 mL), and sodium chloride (10 mL), dried (sodium sulfate), filtered, and concentrated under reduced pressure. The crude product was purified by flash chromatography (silica, 95:5 methylene chloride/methanol) to yield [1-(3,5-Difluoro-benzyl)-3-(6-ethyl-3-hydroxy-chromen-4-ylamino)-2-hydroxy-propyl]-carbamic acid tert-butyl ester (1.30 g, 51%) as a white solid: ¹H NMR (300 MHz, CDCl₃) δ 7.42-7.38 (m, 1H), 7.20-6.96 (m, 1H), 6.78-6.62 (m, 5H), 4.64-4.58 (m, 1H), 4.56-4.20 (m, 1H), 4.18-4.08 (m, 2H), 3.90-3.48 (m, 4H), 3.16-2.70 (m, 5H), 2.64-2.50 (m, 2H), 1.50-1.30 (s, 9H), 1.23-1.18 (m, 3H); ESI MS m/z 493 [C₂₆H₃₄F₂N₂O₅+H].

### Step 2

Hydrogen chloride (4.77 mL, 4 M solution in dioxane, 19.09 mmol) was added to a solution of [1-(3,5-difluoro-benzyl)-3-(6-ethyl-3-hydroxy-chromen-4-ylamino)-2-hydroxy-propyl]-carbamic acid tert-butyl ester (0.47 g, 0.95 mmol) in dioxane (20 mL) at room temperature and the reaction mixture stirred for 17 h. The reaction mixture was concentrated under reduced pressure and the residue triturated with diethyl ether to yield 4-[3-Amino-4-(3,5-difluoro-phenyl)-2-hydroxy-butylamino]-6-ethyl-chromen-3-ol (0.38 g, 85%) as a white solid: ¹H NMR (300 MHz, CD₃OD) δ 7.40 (s, 1H), 7.19-7.17 (m, 1H), 7.05-6.83 (m, 5H), 4.71-4.69 (m, 1H), 4.44-4.40 (m, 2H), 4.19-4.08 (m, 3H), 3.78 (br s, 1H), 3.78-3.52 (m, 1H), 3.49-3.47 (m, 1H), 3.34-3.30 (m,1H), 3.12-3.01 (m 2H), 2.98-2.63 (m, 4H), 1.30-1.17 (m, 3H); ESI MS m/z 393 [C₂₁H₂₆F₂N₂O₃+H].

### Step 3

An additional solution of 4-[3-amino-4-(3,5-difluoro-phenyl)-2-hydroxy-butylamino]-6-ethyl-chromen-3-ol (0.38 g, 0.82 mmol), diisopropylethylamine (0.71 mL, 4.09 mmol) in methylene chloride (5 mL) was added to a suspension of sodium acetate (0.67 g, 0.82 mmol), diisopropylethylamine (0.71 mL, 4.09 mmol) and HBTU (0.31 g, 0.82 mmol) in methylene chloride (5 mL) and the combined mixture was stirred at room temperature for 24 h. Water (30 mL) was added and the aqueous phase was extracted with additional methylene chloride (5 mL). The combined organic phase was washed successively with 0.5 N hydrochloric acid (10 mL) and saturated sodium chloride (10 mL), dried (sodium sulfate), filtered and concentrated under reduced pressure. Purification by preparative HPLC (Phenomenex Luna C18(2) Column, 250×21.20 mm, 10 μ. A: 0.05% TFA in 95:5 H₂O/CH₃CN; B: 0.05% TFA in 5:95 H₂O/CH₃CN. Gradient: 20-95% B over 16 min; flow 19 mL/min. Detection: 220 nm) yielded N-[1-(3,5-Difluoro-benzyl)-3-(6-ethyl-3-hydroxy-chromen-4-ylamino)-2-hydroxy-propyl]-acetamide (55 mg, 4%) as a white foam: IR (ATR) 3254, 2966,1657,1627, 1596 cm−¹; ¹H NMR (300 MHz, CD₃OD) δ 7.34-7.28 (m, 1H), 7.17-7.14 (m, 1H), 6.88-6.75 (m, 5H), 4.56-4.54 (m, 1H), 4.39-4.34 (m, 1H), 4.16-4.04 (m, 3H), 3.90-3.85 (m, 1H), 3.77-3.62 (m, 1H), 3.54-3.10 (m, 5H), 2.71-2.57 (m, 3H), 1.85-1.82 (m, 3H), 1.28-1.16 (m, 3H); ESI MS m/z 435 [C₂₃H₂₈F₂N₂O₄+H]; HPLC (Phenomenex Luna Cl 8(2) Column, 150×4.6 mm, 5μ; A: 0.05% TFA in 95:5 H₂O/CH₃CN; B: 0.05% TFA in 5:95 H₂O/CH₃CN; Gradient: 10-90% B over 15 min; flow 1.0 mL/min; Detection: 225 nm) 94.1 (AUC), t_{R}=11.1, 11.5 min (3:2 mixture of diastereomers).

### EXAMPLE 81

### Examples of Representative Compounds

The following formula (I) compounds can be prepared essentially according to the procedures set forth in the above examples and schemes: N-(1-(3,5-difluorobenzyl)-3-{[6-(2,2-dimethylpropyl)-3,4-dihydro-2H-chromen-4-yl]amino}-2-hydroxypropyl)-2-fluoroacetamide, N-(1-(3,5-difluorobenzyl)-2-hydroxy-3-{[1-(2-isobutyl-1,3-thiazol-5-yl)-1-methylethyl]amino}propyl)acetamide, N-[3-({1-[3-(2-adamantyl)phenyl]cyclohexyl}amino)-1-(3,5-difluorobenzyl)-2-hydroxypropyl]acetamide, N-[3-{[1-(3-cyclopentylphenyl)cyclopropyl]amino}-1-(3,5-difluorobenzyl)-2-hydroxypropyl]acetamide, N-[3-{[1-(3-bicyclo[2.2.1]hept-2-ylphenyl)cyclopropyl]amino}-1-(3,5-difluorobenzyl)-2-hydroxypropyl]acetamide, Ethyl 3-[3-(1-{[3-(acetylamino)-4-(3,5-difluorophenyl)-2-hydroxybutyl]amino}cyclohexyl)phenyl]propanoate, N-[3-{[1-(3-sec-butylphenyl)cyclopropyl]amino}-1-(3,5-difluorobenzyl)-2-hydroxypropyl]acetamide, N-(1-(3,5-difluorobenzyl)-3-{[1-(3′,5′-difluorobiphenyl-3-yl)cyclopropyl]amino}-2-hydroxypropyl)acetamide, N-(1-(3,5-difluorobenzyl)-3-{[5-(2,2-dimethylpropyl)-2-(2-propyl-1H-imidazol-1-yl)benzyl]amino}-2-hydroxypropyl)acetamide, N-[3-{[1-(3-sec-butylphenyl)cyclohexyl]amino}-1-(3,5-difluorobenzyl)-2-hydroxypropyl]acetamide, N-[1-(3,5-difluorobenzyl)-2-hydroxy-3-({1-[3-(3-methylbutyl)phenyl]cyclohexyl}amino)propyl]acetamide, N-[1-(3,5-difluorobenzyl)-3-({1-[3-(1-ethylpropyl)phenyl]cyclohexyl}amino)-2-hydroxypropyl]acetamide, N-[3-{[1-(3-cyclopentylphenyl)cyclohexyl]amino}-1-(3,5-difluorobenzyl)-2-hydroxypropyl]acetamide, N-(1-(3,5-difluorobenzyl)-2-hydroxy-3-{[1-(3-pent-4-en-1-ylphenyl)cyclohexyl]amino}propyl)acetamide, N-(1-(3,5-difluorobenzyl)-2-hydroxy-3-{[1-(3-pyridin-2-ylphenyl)cyclohexyl]amino}propyl)acetamide, N-[1-(3,5-difluorobenzyl)-2-hydroxy-3-({1-[3-(3-methylpyridin-2-yl)phenyl]cyclohexyl}amino)propyl]acetamide, N-[1-(3,5-difluorobenzyl)-2-hydroxy-3-({1-[3-(1,3-thiazol-2-yl)phenyl]cyclohexyl}amino)propyl]acetamide, N-[1-(3,5-difluorobenzyl)-2-hydroxy-3-({1-[3-(3-methyl-2-thienyl)phenyl]cyclohexyl}amino)propyl]acetamide, N-[1-(3,5-difluorobenzyl)-3-({1-[3-(2-fluorobenzyl)phenyl]cyclohexyl}amino)-2-hydroxypropyl]acetamide, N-[1-(3,5-difluorobenzyl)-3-({1-[3-(4-fluorobenzyl)phenyl]cyclohexyl}amino)-2-hydroxypropyl]acetamide, N-[3-{[7-(2,2-dimethylpropyl)-1,2,3,4-tetrahydronaphthalen-1-yl]amino}-1-(3-fluoro-4-hydroxybenzyl)-2-hydroxypropyl]acetamide, N-(1-(3,5-difluorobenzyl)-2-hydroxy-3-{[3-(3-isopropylphenyl)tetrahydro-2H-pyran-3-yl]amino}propyl)acetamide, N-[3-[1-(3-tert-Butyl-phenyl)-4-oxo-cyclohexylamino]-1-(3,5-difluoro-benzyl)-2-hydroxy-propyl]-acetamide, N-[3-[5-(3-tert-Butyl-phenyl)-2-oxo-[1,3]oxazinan-5-ylamino]-1-(3,5-difluoro-benzyl)-2-hydroxy-propyl]-acetamide, N-[3-[5-(3-tert-Butyl-phenyl)-2-oxo-hexahydro-pyrimidin-5-ylamino]-1-(3,5-difluoro-benzyl)-2-hydroxy-propyl]-acetamide, N-[3-[1-(3-Bromo-5-tert-butyl-phenyl)-cyclohexylamino]-1-(3,5-difluoro-benzyl)-2-hydroxy-propyl]-acetamide, N-[3-[1-(3-tert-Butyl-5-ethyl-phenyl)-cyclohexylaminol]-1-(3,5-difluoro-benzyl)-2-hydroxy-propyl]-acetamide, N-[3-[4-(3-tert-Butyl-5-ethyl-phenyl)-tetrahydropyran-4-ylamino]-1-(3,5-difluoro-benzyl)-2-hydroxypropyl]-acetamide, N-[3-[4-(3-Bromo-5-tert-butyl-phenyl)-tetrahydropyran-4-ylamino]-1-(3,5-difluorobenzyl)-2-hydroxypropyl]-acetamide, N-[3-[1-(3-tert-Butyl-5-ethylphenyl)cyclopropylamino]-1-(3,5-difluorobenzyl)-2-hydroxypropyl]-acetamide, N-[3-{1-[3-Bromo-5-(2,2-dimethyl-propyl)-phenyl]-cyclopropylamino}-1-(3,5-difluoro-benzyl)-2-hydroxy-propyl]-acetamide, N-(1-(3,5-Difluorobenzyl)-3-{1-[5-(2,2-dimethylpropyl)-2-imidazol-1-yl-phenyl]-cyclopropylamino}-2-hydroxy-propyl)-acetamide, N-{1-(3,5-Difluoro-benzyl)-3-[5-(2,2-dimethylpropyl)-2-(5-ethyl-imidazol-1-yl)-benzylamino]-2-hydroxypropyl}-acetamide, N-[3-[3-Chloro-5-(2,2-dimethyl-propyl)-2-imidazol-1-yl-benzylamino]-1-(3,5-difluoro-benzyl)-2-hydroxy-propyl]-acetamide, N-{1-(3,5-Difluoro-benzyl)-3-[5-(2 ,2-dimethyl-propyl)-2-tetrazol-1-yl-benzylamino]-2-hydroxy-propyl}-acetamide, N-{1-(3,5-Difluoro-benzyl)-3-[6-(2,2-dimethyl-propyl)-thiochromen-4-ylamino]-2-hydroxy-propyl}-acetamide, N-{1-(3,5-Difluoro-benzyl)-3-[6-(2,2-dimethyl-propyl)-8-ethyl-chromen-4-ylamino]-2-hydroxy-propyl}-acetamide, N-[3-[8-Bromo-6-(2,2-dimethylpropyl)-chromen-4-ylamino]-1-(3,5-difluoro-benzyl)-2-hydroxypropyl]-acetamide, N-{1-(3,5-Difluoro-benzyl)-3-[6-(2,2-dimethyl-propyl)-2-oxo-chromen-4-ylamino]-2-hydroxy-propyl}-acetamide, N-(1-(3,5-difluorobenzyl)-3-{[6-(2,2-dimethylpropyl)-3,4-dihydro-2H-chromen-4-yl]amino}-2-hydroxypropyl)acetamide, N-[3-{[6-(2,2-dimethylpropyl)-3,4-dihydro-2H-chromen-4-yl]amino}-1-(3-fluorobenzyl)-2-hydroxypropyl]acetamide, N-(1-(3,5-difluorobenzyl)-3-{[6-(2,2-dimethylpropyl)-4-methyl-3,4-dihydro-2H-chromen-4-yl]amino}-2-hydroxypropyl)acetamide, N-(1-(3-fluoro-4-hydroxybenzyl)-2-hydroxy-3-{[1-(3-isopropylphenyl)cyclohexyl]amino}propyl)acetamide, N-(1-(3,5-difluorobenzyl)-2-hydroxy-3-{[1-(3-isopropylphenyl)cyclohexyl]amino}propyl)-2-fluoroacetamide, N-(1-[3-(allyloxy)-5-fluorobenzyl]-2-hydroxy-3-{[1-(3-isopropylphenyl)cyclohexyl]amino}propyl)acetamide, N-[1-(3,5-difluorobenzyl)-3-({1-[3-(2,2-dimethylpropyl)phenyl]-1-methylethyl}amino)-2-hydroxypropyl]-2-fluoroacetamide, N-(1-(3,5-difluorobenzyl)-3-{[7-(2,2-dimethylpropyl)-1,2,3,4-tetrahydronaphthalen-1-yl]amino}-2-hydroxypropyl)-2-fluoroacetamide, N-[1-(3,5-difluorobenzyl)-2-hydroxy-3-({1-[3-(3-thienyl)phenyl]cyclohexyl}amino)propyl]acetamide, N-[1-(3,5-difluorobenzyl)-3-({1-[4-(2,2-dimethylpropyl)pyridin-2-yl]cyclopropyl}amino)-2-hydroxypropyl]acetamide, N-(1-(3,5-difluorobenzyl)-2-hydroxy-3-{[7-propyl-1,2,3,4-tetrahydronaphthalen-1-yl]amino}propyl)acetamide, N-(1-(3,5-difluorobenzyl)-2-hydroxy-3-{[1-(3-isobutylphenyl)cyclohexyl]amino}propyl)acetamide, N-(2-hydroxy-1-(4-hydroxybenzyl)-3-{[1-(3-isopropylphenyl)cyclohexyl]amino}propyl)acetamide, N-(1-(3,5-difluorobenzyl)-3-{[7-ethyl-1,2,3,4-tetrahydronaphthalen-1-yl]amino}-2-hydroxypropyl)-2-ethoxyacetamide, N-(1-(3,5-difluorobenzyl)-3-{[(1R)-7-ethyl-1,2,3,4-tetrahydronaphthalen-1-yl]amino}-2-hydroxypropyl)-2,2-difluoroacetamide, N-{1-(3,5-Difluoro-benzyl)-2-hydroxy-3-[1-(3-isopropyl-phenyl)-cyclobutylamino]-propyl}-acetamide, N-{1-(3,5-Difluoro-benzyl)-2-hydroxy-3-[1-(3-isopropyl phenyl)-cyclopentylamino]-propyl}-acetamide, N-{1-(3,5-Difluoro-benzyl)-2-hydroxy-3-[3-(3-isopropyl-phenyl)-bicyclo[3.1.0]hex-3-ylamino]-propyl}-acetamide, N-{1-(3,5-Difluoro-benzyl)-2-hydroxy-3-[3-(3-isopropyl-phenyl)-6-aza-bicyclo[3.1.0]hex-3-ylamino]-propyl}-acetamide, N-{1-(3,5-Difluoro-benzyl)-2-hydroxy-3-[3-(3-isopropyl-phenyl)-6-methyl-6-aza-bicyclo[3.1.0]hex-3-ylamino]-propyl)-acetamide, N-[3-[6-Acetyl-3-(3-isopropyl-phenyl)-6-aza-bicyclo[3.1.0]hex-3-ylamino]-1-(3,5-difluoro-benzyl)-2-hydroxy-propyl]-acetamide, N-{1-(3,5-Difluoro-benzyl)-2-hydroxy-3-[3-(3-isopropyl-phenyl)-6-methanesulfonyl-6-aza-bicyclo[3.1.0]hex-3-ylamino]-propyl)-acetamide, N-{1-(3,5-Difluoro-benzyl)-2-hydroxy-3-[1-(3-isopropyl-phenyl)-2,2,4,4-tetramethyl-3-oxo-cyclobutylamino]-propyl}-acetamide, N-{1-(3,5-Difluoro-benzyl)-2-hydroxy-3-[3-hydroxy-1-(3-isopropyl-phenyl)-2,2,4,4-tetramethyl-cyclobutylamino]-propyl}-acetamide, N-{1-(3,5-Difluoro-benzyl)-2-hydroxy-3-[5-(3-isopropyl-phenyl)-octahydro-cyclopenta[c]pyrrol-5-ylamino]-propyl}-acetamide, N-{1-(3,5-Difluoro-benzyl)-2-hydroxy-3-[5-(3-isopropyl-phenyl)-2-methyl-octahydro-cyclopenta[c]pyrrol-5-ylamino]-propyl)-acetamide, N-[3-[2-Acetyl-5-(3-isopropyl-phenyl)-octahydro-cyclopenta[c]pyrrol-5-ylamino]-1-(3,5-difluoro-benzyl)-2-hydroxy-propyl]-acetamide, N-{1-(3,5-Difluoro-benzyl)-2-hydroxy-3-[5-(3-isopropyl-phenyl)-2-methanesulfonyl-octahydro-cyclopenta[c]pyrrol-5-ylamino]-propyl}-acetamide, N-{1-(3,5-Difluoro-benzyl)-2-hydroxy-3-[2-(3-isopropyl-phenyl)-5-oxo-octahydro-pentalen-2-ylamino]-propyl}-acetamide, N-{1-(3,5-Difluoro-benzyl)-2-hydroxy-3-[5-hydroxy-2-(3-isopropyl-phenyl)-octahydro-pentalen-2-ylamino]-propyl}-acetamide, N-{1-(3,5-Difluoro-benzyl)-2-hydroxy-3-[2-(3-isopropyl-phenyl)-3a,6a-dimethyl-5-oxo-octahydro-pentalen-2-ylamino]-propyl}-acetamide, N-{1-(3,5-Difluoro-benzyl)-2-hydroxy-3-[5-hydroxy-2-(3-isopropyl-phenyl)-3a,6a-dimethyl-octahydro-pentalen-2-ylamino]-propyl}-acetamide, N-{1-(3,5-Difluoro-benzyl)-2-hydroxy-3-[2-(3-isopropyl-phenyl)-5-oxo-cyclohexyl amino]-propyl}-acetamide, N-{1-(3,5-Difluoro-benzyl)-2-hydroxy-3-[5-hydroxy-2-(3-isopropyl-phenyl)-5-methyl-cyclohexylamino]-propyl}-acetamide, N-{1-(3,5-Difluoro-benzyl)-2-hydroxy-3-[2-(3-isopropyl-phenyl)-5-methanesulfonyl amino-cyclohexylamino]-propyl}-acetamide, N-[3-[5-Acetylamino-2-(3-isopropyl-phenyl)-cyclohexylamino]-1-(3,5-difluoro-benzyl)-2-hydroxy-propyl]-acetamide, N-{1-(3,5-Difluoro-benzyl)-2-hydroxy-3-[2-(3-isopropyl-phenyl)-4-oxo-cyclohexyl amino]-propyl}-acetamide, N-{1-(3,5-Difluoro-benzyl)-2-hydroxy-3-[4-hydroxy-2-(3-isopropyl-phenyl)-4-methyl-cyclohexylamino]-propyl}-acetamide, N-[3-[4-Acetylamino-2-(3-isopropyl-phenyl)-cyclohexylamino]-1-(3,5-difluoro-benzyl)-2-hydroxy-propyl]-acetamide, N-1-(3,5-Difluoro-benzyl)-2-hydroxy-3-[2-(3-isopropyl-phenyl)-4-methanesulfonyl amino-cyclohexylamino]-propyl}-acetamide, N-{1-(3,5-Difluoro-benzyl)-2-hydroxy-3-[2-(3-isopropyl-phenyl)-4-oxo-cyclopentyl amino]-propyl}-acetamide, N-{1-(3,5-Difluoro-benzyl)-2-hydroxy-3-[4-hydroxy-2-(3-isopropyl-phenyl)-4-methyl-cyclopentylamino]-propyl}-acetamide, N-[3-[4-Acetylamino-2-(3-isopropyl-phenyl)-cyclopentylamino]-1-(3,5-difluoro-benzyl)-2-hydroxy-propyl]-acetamide, N-{1-(3,5-Difluoro-benzyl)-2-hydroxy-3-[2-(3-isopropyl-phenyl)-4-methanesulfonyl amino-cyclopentylamino]-propyl}-acetamide, N-{1-(3,5-Difluoro-benzyl)-3-[4-(2,2-dimethyl-propyl)-pyridin-3-ylamino]-2-hydroxy-propyl}-acetamide, N-(1-(3,5-Difluoro-benzyl)-3-{[4-(2,2-dimethyl-propyl)-pyridin-3-ylmethyl]-amino}-2-hydroxy-propyl)-acetamide, N-[1-(3,5-Difluoro-benzyl)-2-hydroxy-3-(5-isobutyl-2-piperazin-1-yl-benzylamino)-propyl]-acetamide, N-{1-(3,5-Difluoro-benzyl)-2-hydroxy-3-[5-isobutyl-2-(4-methyl-piperazin-1-yl)-benzylamino]-propyl}-acetamide, N-[3-[2-(4-Acetyl-piperazin-1-yl)-5-isobutyl-benzylamino]-1-(3,5-difluoro-benzyl)-2-hydroxy-propyl]-acetamide, N-[3-[6-(2,2-Dimethyl-propyl)-chromen-4-ylamino]-1-(3-fluoro-4-hydroxy-benzyl)-2-hydroxy-propyl]-acetamide, N-[3-[6-(2,2-Dimethyl-propyl)-chromen-4-ylamino]-2-hydroxy-1-(5-hydroxy-pyridin-2-ylmethyl)-propyl]-acetamide, N-{1-(3,1-Difluoro-benzyl)-3-[6-(2,2-dimethyl-propyl)-chromen-4-ylamino]-2-hydroxy-propyl}-2-fluoro-acetamide, N-{1-(3,5-difluorobenzyl)-2-hydroxy-3-[(6-iodo-3,4-dihydro-2H-chromen-4-yl)amino]propyl}acetamide, N-(1-(3,5-difluorobenzyl)-2-hydroxy-3-{[6-iodo-3,4-dihydro-2H-chromen-4-yl]amino}propyl)acetamide, N-(1-(3,5-difluorobenzyl)-2-hydroxy-3-{[6-iodo-3,4-dihydro-2H-chromen-4-yl]amino}propyl)acetamide, N-(1-(3,5-difluorobenzyl)-3-{[6-ethyl-3,4-dihydro-2H-chromen-4-yl]amino}-2-hydroxypropyl)acetamide, N-(1-(3,5-difluorobenzyl)-3-{[6-ethyl-3,4-dihydro-2H-chromen-4-yl]amino}-2-hydroxypropyl)acetamide, N-(1-(3,5-difluorobenzyl)-2-hydroxy-3-{[6-(1H-pyrrol-3-yl)-3,4-dihydro-2H-chromen-4-yl]amino}propyl)acetamide, N-{1-(3,5-difluorobenzyl)-2-hydroxy-3-[(6-isopropyl-3,4-dihydro-2H-chromen-4-yl)amino]propyl}acetamide, N-[1-(3,5-difluorobenzyl)-3-(3,4-dihydro-2H-chromen-4-ylamino)-2-hydroxypropyl]acetamide, N-(1-(3,5-difluorobenzyl)-2-hydroxy-3-{[6-isobutyl-3,4-dihydro-2H-chromen-4-yl]amino}propyl)acetamide, N-[3-{[6-cyano-3,4-dihydro-2H-chromen-4-yl]amino}-1-(3,5-difluorobenzyl)-2-hydroxypropyl]acetamide, N-(1-(3,5-difluorobenzyl)-2-hydroxy-3-{[6-neopentyl-3,4-dihydro-2H-chromen-4-yl]amino}propyl)acetamide, N-[3-(6-tert-Butyl-chroman-4-ylamino)-1-(3,5-difluoro-benzyl)-2-hydroxy-propyl]-acetamide, N-[3-(6-tert-Butyl-chroman-4-ylamino)-1-(3-fluoro-benzyl)-2-hydroxy-propyl]-acetamide, N-[3-(6-tert-Butyl-1,2,3,4-tetrahydro-quinolin-4-ylamino)-1-(3,5-difluoro-benzyl)-2-hydroxy-propyl]-acetamide, and N-{1-(3,5-difluorobenzyl)-2-hydroxy-3-[(6-neopentyl-3,4-dihydro-2H-chromen-4-yl)amino]propyl}acetamide.

### EXAMPLE 82

### Scheme for Hydroxyl Replacement

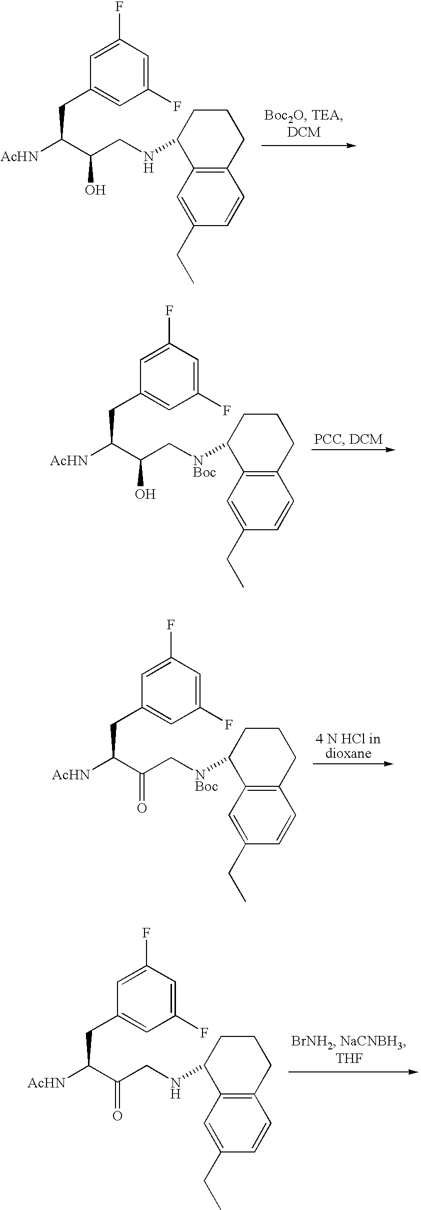

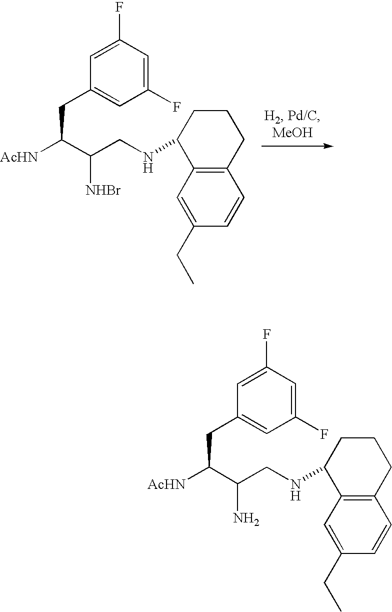

### EXAMPLE 83

### Alternative Scheme for Hydroxyl Replacement

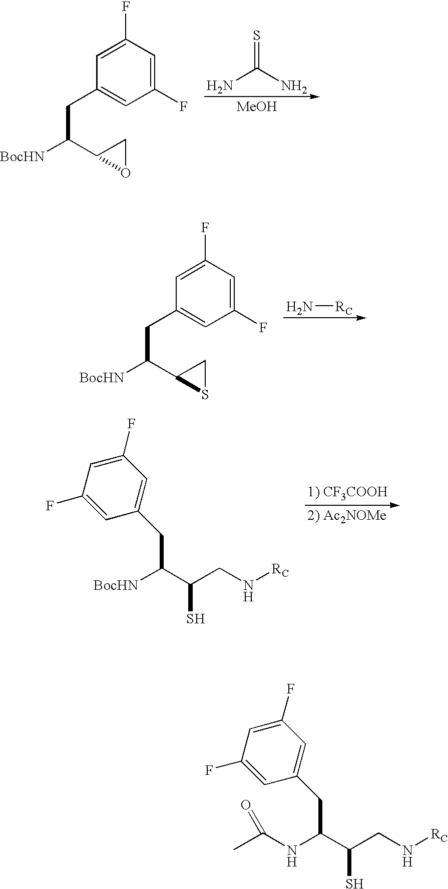

### EXAMPLE 84

### ALTERNATIVE PREPARATION OF [2-(3,5-DIFLUORO-PHENYL)-1-OXIRANYL-ETHYL]-CARBAMIC ACID TERT-BUTYL ESTER

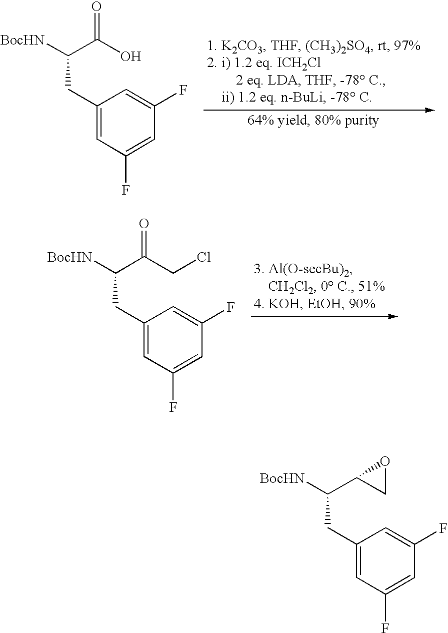

The synthesis of tert-butyl (1S)-2-(3,5-difluorophenyl)-1-[(2S)-oxiranyl]ethylcarbamate was carried out using the procedure described by Reeder in WO2002085877. (2S)-2-[(tert-Butoxycarbonyl)amino]-3-(3,5-difluorophenyl)propionic acid was purchased from Chem Impex and converted to the methyl ester without incident. Conversion of the methyl ester to the chloroketone was carried out on a 50 g scale and repeatedly resulted in yields between 60-65% of an impure product. The chlorohydrin was obtained via a diastereoselective Meerwein-Ponndorf-Verley reduction. The product was washed with octane to remove some, but not all, of the impurities. Conversion of the chlorohydrin to the epoxide occurred with potassium hydroxide in ethanol with the product being isolated from the reaction mixture by precipitation after the addition of water. The epoxide could be recrystallized from hexanes/isopropanol, although some batches of epoxide contained an unidentified impurity.

### Step 1: Preparation of (2S)-2-[(tert-Butoxycarbonyl)amino]-3-(3,5-difluorophenyl)propionic acid methyl ester

A solution of (2S)-2-[(tert-butoxycarbonyl)amino]-3-(3,5-difluorophenyl)propionic acid (138 g, 458 mmol) was dissolved in THF (1000 mL) and cooled to 0° C. Potassium carbonate (69.6 g, 503.8 mmol) was added followed by the dropwise addition of dimethyl sulfate (45.5 mL, 480.9 mmol). The reaction was removed from the ice bath and allowed to stir at room temperature overnight after which HPLC analysis shows the complete consumption of starting material. The reaction was quenched by the addition of 10% ammonium hydroxide (150 mL). The aqueous layer was removed and extracted with ethyl acetate (500 mL). The combined organics were washed with brine (500 mL), dried (magnesium sulfate), and concentrated to give a yellow solid. The solid was recrystallized from hexanes to give the product as an off white solid (140.3 g, 445.0 mmol, 97%).

### Step 2: tert-Butyl (1S)-3-chloro-1-(3,5-difluorobenzyl)-2-oxopropylcarbamate

A solution of LDA was prepared by adding n-BuLi (26 mL, 260 mmol) to a solution of diisopropylamine (26.3 g, 260 mmol) in THF (200 mL) at −78° C. After the addition was complete, the reaction was allowed by warm to 0° C. This light yellow solution was added dropwise to a solution of (2S)-2-[(tert-butoxycarbonyl)amino]-3-(3,5-difluorophenyl)propionic acid methyl ester (40 g, 127 mmol) and chloroiodomethane (11.1 mL, 152 mmol) at −65° C. or colder. After the addition, the solution was stirred for 30 min at −78° C. n-BuLi (15 mL, 150 mmol) was added dropwise at −62° C. or colder. The reaction was stirred for 30 min at −78° C. then quenched into 500 mL of 1N HCl at 0° C. The product was extracted into EtOAc (500 mL), washed with brine, dried (magnesium sulfate), filtered, and concentrated. Octane (400 mL) was added to the product and the resulting solid collected by filtration and dried. The octane was cooled to −78° C. then allowed to warm until the octane melted. The resulting solid was collected and added to the previously collected solid. Drying of the combined solid yielded the title compound as an off-white solid (33.9 g, 101.5 mmol, 64.5 %).

### Step 3: tert-Butyl (1S,2S)-3-chloro-1-(3,5-diflurorbenzyl)-2-hydroxypropylcarbamate

A solution of tert-butyl (1S)-3-chloro-1-(3,5-difluorobenzyl)-2-oxopropylcarbamate (67.4 g, 202 mmol) was dissolved in DCM (500 mL) and cooled to 0° C. Tri(sec-butoxy)aluminum (54.7 g, 222.1 mmol, 1.1 eq) in DCM (50 mL) was added dropwise. After stirring for 2 h at 0° C., the reaction was complete by HPLC. The reaction was quenched with 1N HCl (750 mL) and the product was extracted into ethyl acetate (2×400 mL), washed with brine, dried (magnesium sulfate), filtered, and concentrated to give an oily yellow solid. Octane (300 mL) was added and the resulting solid was collected by filtration and washed with octane. Drying overnight yielded a white solid. The octane layers were collected and concentrated to about 100 mL of volume, then placed in the freezer for 48 h to yield a second crop of the title compound (35 g, 104 mmol, 51%)

### Step 4: tert-Butyl (1S)-2-(3,5-diflurorphenyl)-1-[(2S)-oxiranyl]ethylcarbamate

A solution of tert-butyl (1S,2S)-3-chloro-1-(3,5-diflurorbenzyl)-2-hydroxypropylcarbamate in ethanol (150 mL) was cooled to 0° C. A solution of KOH in EtOH (25 mL) was added. The reaction was removed from the ice bath and stirred for 2 h. The reaction was diluted with 300 mL of water and placed into an ice bath. The resulting solid was collected by filtration and washed with cold water (100 mL). Drying overnight yielded an off-white solid (6.74 g, 22.51 mmol, 90%).

### EXAMPLE 85

### ALTERNATIVE PREPARATION OF 4-AMINO-6-(2,2-DIMETHYL-PROPYL)-3,4-DIHYDRO-2H-QUINOLINE-1-CARBOXYLIC ACID BENZYL ESTER

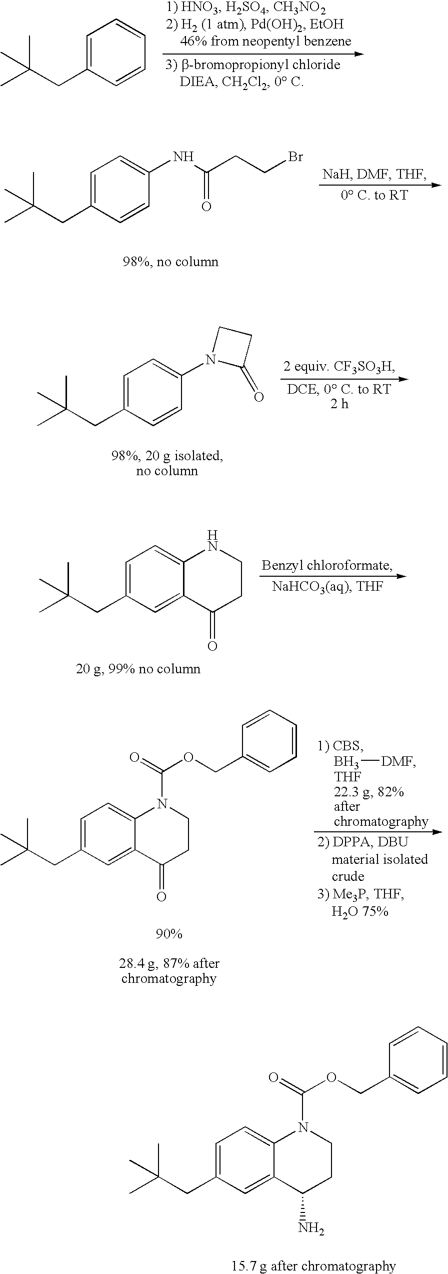

### Step 1: 1-(2,2-Dimethyl-propyl)-4-nitro-benzene and 1-(2,2-Dimethyl-propyl)-2-nitro-benzene

Concentrated HNO₃ (11.6 mL) was added dropwise to a stirred solution of concentrated sulfuric acid (13.8 mL) at 0° C. The mixture was added dropwise to a solution of neopentyl benzene (17.2 g, 116 mmol) in nitromethane (90 mL) stirring at 0° C. The temperature warmed to about 3° C. during the dropwise addition of the acid mixture. After warming to room temperature and stirring overnight, the reaction was poured into 400 mL ice water and extracted with CH₂Cl₂. The combined organics were washed with H₂O, saturated NaHCO₃, and brine. The organics were dried (magnesium sulfate), filtered, and concentrated to a yellow oil corresponding to ¹H-NMR which appears to be about a 1:1 mixture of regio-isomers. This mixture was used crude in the subsequent reduction.

### Step 2: 4-(2,2-Dimethyl-propyl)-phenylamine

Pearlman's catalyst (4 g) was added to a stirred solution of the mixture of nitro compounds (22.4 g, 116 mmol) in 300 mL 95% EtOH. The suspension was vacuum purged with H₂(g) and then held under 1 atm H₂ overnight. TLC in 9/1 hexanes/EtOAc showed two new lower rf spots. The reaction was filtered through GF/F filter paper with 95% EtOH and the filtrate was concentrated. The crude material was loaded onto a Biotage 75 L column with 5/95 EtOAc/hexanes and eluted first with 5/95 EtOAc/hexanes (4 L) followed by 1/9 EtOAc/hexanes (6 L). The two regioisomeric anilines separated and were concentrated to give the undesired high rf aniline as an orange oil and the desired lower rf aniline as a tan solid (8.7 g, 46% from neopentyl benzene; ¹H NMR (400 MHz, CDCl₃) δ 6.91 (d, J=6.4 Hz, 1 H), 6.61 (d, J=6.4 Hz, 1H), 3.54 (s, 2H), 2.38 (s, 2H), 0.87 (s, 9H); LC rt=2.89 min).

### Step 3: 3-Bromo-N-[4-(2,2-dimethyl-propyl)-phenyl]-propidnamide

Dimethylaniline (12.5 g, 103 mmol), followed by β-bromopropionyl chloride (17.68 g, 103 mmol), was added to a stirred solution of the aniline (15.3 g, 93.78 mmol) in CH₂Cl₂ (300 mL) at 0° C. under N₂(g). After 2 h, the reaction was diluted to 400 mL with CH₂Cl₂ and washed with 2N HCl, saturated NaHCO₃, and brine. The organics were dried (magnesium sulfate), filtered, and concentrated to a white solid (27.5 g, 98%) corresponding to ¹H NMR and HPLC showing a 10% impurity which is the beta chloro compound (LC rt=4.06 min). The mixture was taken to the next step without further purification.

### Step 4: 1-[4-(2,2-Dimethyl-propyl)-phenyl]-azetidin-2-one

Sodium hydride (60% oil dispersion, 4.61 g, 115 mmol) was added to a stirred solution of DMF (115 mL) at 0° C. under N₂(g). The P-bromoamide (27.5 g, 92 mmol) in THF (270 mL) was then added dropwise by cannulation. The cooling bath was allowed to slowly melt and the reaction was stirred at room temperature overnight. The white suspension was then partitioned between EtOAc (400 mL) and brine (300 mL). The organics were isolated, washed with brine, dried (magnesium sulfate), filtered, and concentrated to an off white solid (20 g, 100%; LC rt=3.87 min). The crude product was used in the following reaction.

### Step 5: 6-(2,2-Dimethyl-propyl)-2,3-dihydro-1H-quinolin-4-one

Triflic acid (27.76 g, 185 mmol) was added drop-wise to a stirred solution of the β-lactam (20.1 g, 92.5 mmol) in 300 mL dichloroethane at 0° C. under N₂(g). The reaction was stirred for 4 h at room temperature. The reaction was poured into 1 L of stirred 1:1 CH₂Cl₂:ice cold saturated NaHCO₃. The product was extracted with CH₂Cl₂, dried (magnesium sulfate), filtered, and concentrated to a yellow oil (20.1 g, 100%), which was used without further purification in the CBz protection.

### Step 6: 6-(2,2-Dimethyl-propyl)-4-oxo-3,4-dihydro-2H-quinoline-1-carboxylic acid benzyl ester

To a stirred solution of the tetrahydroquinolone (20.1 g, 92.5 mmol) in 300 mL CH₂Cl₂ at 0° C. under N₂(g) was added DIEA (23.9 g, 185 mmol) by syringe followed by benzyl chloroformate (23.7 g, 139 mmol) dropwise by addition funnel. The reaction was allowed to warm to room temperature overnight, washed with 2N HCl and saturated NaHCO₃. The organics were dried (magnesium sulfate), filtered, and concentrated to a brown oil which was loaded directly onto a Biotage 75 L column and eluted with 9/1 hexanes/EtOAc. Product containing fractions were pooled and concentrated to a pale yellow oil that solidified upon standing (28.4 g, 87% from the aniline).

### Step 7: 6-(2,2-Dimethyl-propyl)-4-(R)-hydroxy-3,4-dihydro-2H-quinoline-1-carboxylic acid benzyl ester

The CBS reagent (1M in toluene, 7.9 mL, 7.9 mmol) was added to a stirred solution of the ketone (27.5 g, 79 mmol) in 79 mL THF at −25° C. (CCl₄/dry ice bath) under N₂(g). Then borane dimethylsulfide complex (2M in THF, 39.5 mL, 79 mmol) diluted with 95 mL THF was added at −20° C. or colder. After 1 h, the reaction was allowed to warm to room temperature and was stirred overnight. The reaction was recooled to 0° C. and quenched by addition of 190 mL MeOH via addition funnel. After removal of the cooling bath and stirring at room temperature for 2 h, the reaction was concentrated to dryness by rotovap and high vacuum and then loaded onto a Biotage 75 M column with 4/1 hexanes/EtOAc and eluted. Product containing fractions were pooled and concentrated to a pale yellow oil that solidified upon standing (22.3 g, 80%). H NMR (400 MHz, CDCl₃) δ 7.78 (d, J=8.2 Hz, 1H), 7.43-7.29 (m, 5H), 7.13 (d, J=1.8 Hz, 1H), 7.03 (dd, J=8.2, 1.8 Hz, 1H), 5.24 (AB q, J=12.5 Hz, 2H), 4.75 (q, J=4.7 Hz, 1H), 4.19-4.09 (m, 1H), 3.68 (ddd, J=13.3, 9.5, 4.0 Hz, 1H), 2.46 (s, 2H), 2.14-1.97 (m, 2H), 1.71 (d, J=5.0 Hz, 1H), 0.90 (s, 9H).

### Step 8: 4-(S)-Azido-6-(2,2-dimethyl-propyl)-3,4-dihydro-2H-quinoline-1-carboxylic acid benzyl ester

DPPA (20.84 g, 75.7 mmol) was added to a stirred solution of the alcohol (22.3 g, 63 mmol) in 126 mL toluene at 0° C. under N₂(g). DBU (11.53 g, 75.7 mmol) in toluene was then added dropwise. The reaction was allowed to warm to room temperature and stirred overnight. The reaction was reduced to about 100 mL by rotovap and was then loaded onto a Biotage 75 M column with minimum CH₂Cl₂ and eluted with 5/95 EtOAc/hexanes. The product containing fractions were pooled and concentrated to a clear oil which solidified upon standing (22 g, 92%).

### Step 9: 4-(S)-Amino-6-(2,2-dimethyl-propyl)-3,4-dihydro-2H-quinoline-1-carboxylic acid benzyl ester

H₂O (1.26 g, 70 mmol) was added to a stirred solution of the azide (22 g, 58 mmol) in 580 mL THF at room temperature under N₂(g). Trimethylphosphine (1M in toluene, 67 mL, 67 mmol) was then added and the reaction was stirred overnight. The reaction was concentrated to a yellow oil by rotary evaporation followed by high vacuum. The crude material was dissolved in EtOAc and the resulting precipitate was filtered off and discarded. The crude product filtrate was loaded onto a Biotage 75 M column with EtOAc and eluted with the same solvent. Product containing fractions were pooled and concentrated to a pale yellow oil (15.7 g, 77%). ¹H NMR (400 MHz, CDCl₃) δ 7.68 (d, J=8.0 Hz, 1H), 7.43-7.28 (m, 5H), 7.09 (s, 1H), 6.97 (d, J=8.1 Hz, 1H), 5.24 (AB q, J=12.5 Hz, 2H), 4.01-3.91 (m, 2H), 3.84-3.76 (m, 1H), 2.45 (s, 2H), 2.19-2.09 (m, 1 H), 1.82-1.72 (m, 1H), 0.90 (s, 9H); LC rt=3.18 min.

### EXAMPLE 86

### SYNTHESIS OF N-{1-(3,5-DIFLUORO-BENZYL)-3-[6-(2,2-DIMETHYL-PROPYL)-1-METHYL-1,2,3,4-TETRAHYDRO-QUINOLIN-4-YLAMINO]-2-HYDROXY-PROPYL}-ACETAMIDE

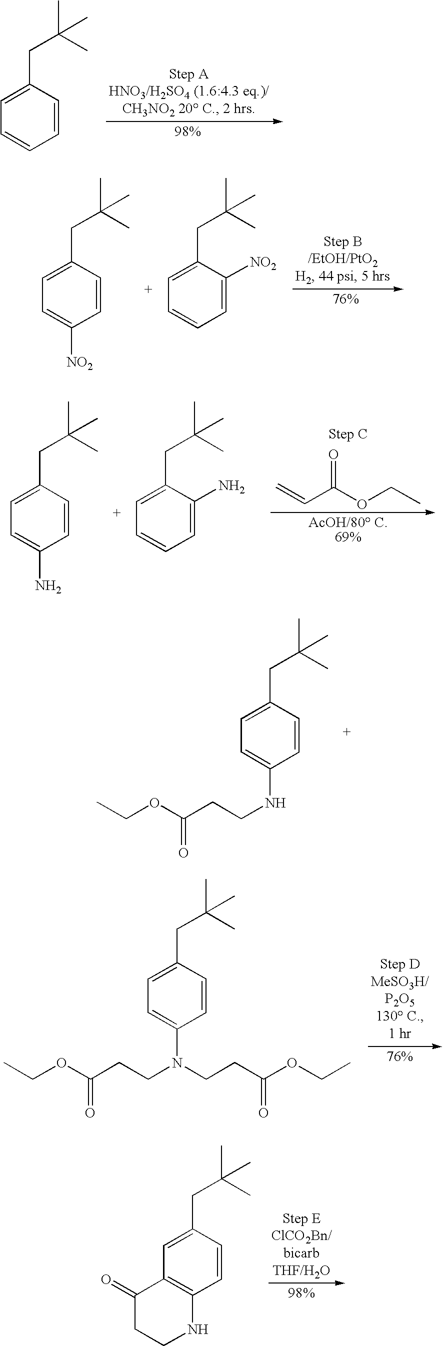

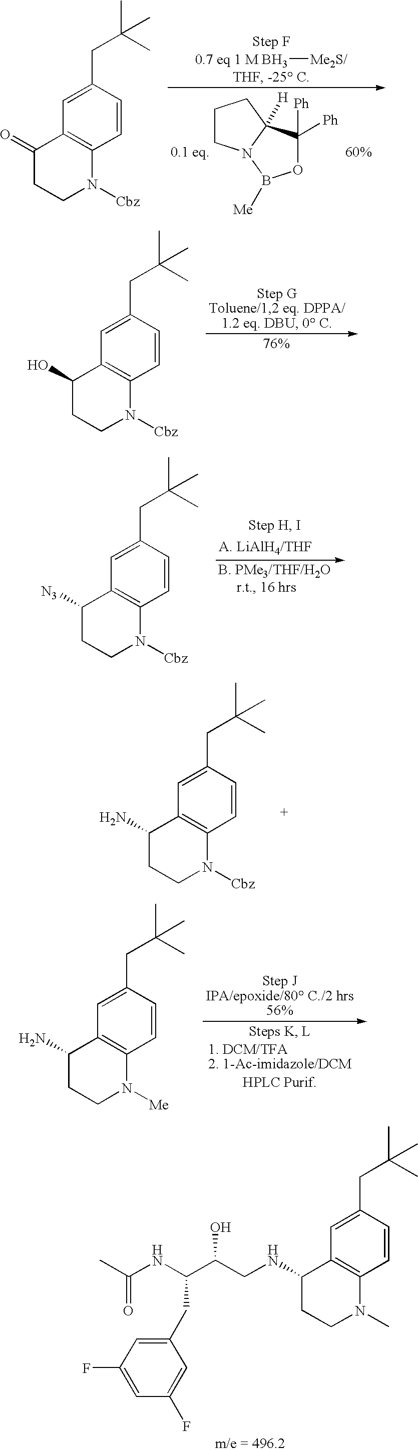

### Step A. 1-Isobutyl-4-nitro-benzene and 1-(2,2-Dimethyl-propyl)-2-nitro-benzene

5.8 mL (92 mmol, 1.6 eq.) of conc'd nitric acid at 0° C. was added dropwise to 6.9 mL (249 mmol, 4.3 eq.) of conc'd sulfuric acid in 10 min. The mixture was then added to 8.6 g (57.9 mmol) of 2,2-di-methyl-propylbenzene in 45 mL of nitromethane slowly, stirred at 0° C. for 2 hrs and overnight at room temperature. The reaction was monitored by TLC, two new spots appeared at Rf=0.63 and 0.59.

The mixture was poured into ice slowly and extracted with dichloromethane 3× and the combined extractants were washed with bicarb, brine and water successively, dried with anhydrous sodium sulfate. The solvents were stripped, yielding 11.02 g of an oil, a mixture of ortho and para isomers (98%).

TLC (10% EtOAc/Hexane) Rf=0.63 and 0.59 while starting material at Rf=0.91. LCMS m/e=194.1(M+H), Rt=2.693 min (50% [B]: 50% [A] to 95% [B]: 5% [A] gradient in 3.33 min, then hold, at 1.5 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.).

### Step B. 4-(2,2-Dimethyl-propyl)-phenylamine and 2-(2,2-Dimethyl-propyl)-phenylamine

240 mg (1.05 mmol, 4.2 mg/mmol) of platinum(IV) oxide was added to 11.0 g (57 mmol) of the product in Step A in 20 mL of ethanol. The mixture was then saturated with hydrogen at 44 psi and shaken for 4 h. The mixture was then filtered through celite and the filtrates combined and stripped to give 9.26 g of the crude mixture, which was purified by flash column to give 3.47 g of a burgendy oil (o-isomer, 37%) and 3.92 g as a beige solid (p-isomer, 42%).

TLC (20% EtOAc/Hexane) Rf=0.65 and 0.48 while starting material at Rf=0.85 and 0.82. LCMS m/e=164.1(M+H), Rt=1.937 (20% [B]: 80% [A] to 70% [B]: 30% [A] gradient in 2.33 min, then hold, at 1.5 mumin, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.).

### Step C. 3-[4-(2,2-Dimethyl-propyl)-phenylamino]-propionic acid ethyl ester and 3-[[4-(2,2-Dimethyl-propyl)-phenyl]-(2-ethoxycarbonyl-ethyl)-amino]-propionic acid ethyl ester

3.2 g (32 mmol, 1 eq.) of ethyl acrylate was added to 5.21 g of the para isomeric product in Step B (32 mmol) in 8 mL of acetic acid. The mixture was then heated to 80° C. for 2 h and kept at 55° C. overnight.

The reaction was monitored by TLC and two new spots appeared at Rf=0.67 and 0.61. The mixture was partitioned by EtOAc/brine and dried over anhydrous sodium sulfate. Stripping the solvent gives 8.94 g of crude product, which was purified by flash column to give 3.47 g as a burgundy oil (69%).

TLC (20% EtOAc/Hexane) Rf=0.67 and 0.61 while starting material at Rf=0.47. LCMS m/e=264.2(M+H), Rt=2.639 min and LCMS m/e=364.2(M+H), Rt=3.524 min (using the method described in Step B.)

### Step D. 6-(2,2-Dimethyl-propyl)-2,3-dihydro-1H-quinolin-4-one

4.9 g of phosphorus pentoxide (17.3 mmol, 1.3 eq.) was dissolved in 49 mL of methanesulfonic acid (756 mmol, 56 eq.) at 130° C. The mixture was allowed to cool to room temperature. 6.57 g of the product of Step C (13.5 mmol) was then added to the mixture. The reaction was heated to 130° C. for 1 h and monitored by TLC; a new spot appeared at Rf=0.61.

The mixture was slowly poured into ice and treated with 1 N NaOH to pH=10, then extracted with dichloromethane 3×, and the combined extractants washed with brine and dried with anhydrous sodium sulfate. The solvent was stripped and the crude product was purified by flash column yielding 4.97 g as a tan oil, which was solidified upon standing (76%).

TLC (50% EtOAc/Hexane) Rf=0.61 while starting material at Rf=0.91 and 0.89. LCMS m/e=218.1(M+H), Rt=3.006 min (Using the method in Step B.)

### Step E. 6-(2,2-Dimethyl-propyl)-4-oxo-3,4-dihydro-2H-quinoline-1-carboxylic acid benzyl ester

4.8 g of the product of Step D in 30 mL of THF was added to 5.6 g of sodium bicarbonate (66 mmol, 3 eq.) dissolved in 10 mL of water. 4.12 g of benzyl chloroformate in 5 mL of THF was added slowly to the mixture at 0° C. The mixture was stirred at room temperature for 2 h and the reaction was monitored by TLC; a new spot appeared at Rf=0.86.

The mixture was extracted with ether 3× and washed with 5% citric acid and brine successively. The mixture was then dried with anhydrous sodium sulfate and the solvent was stripped yielding 7.69 g of 6-(2,2-Dimethyl-propyl)-4-oxo-3,4-dihydro-2H-quinoline-1-carboxylic acid benzyl ester as a tan oil (98%).

TLC (50% EtOAc/Hexane) Rf=0.86 (blue color under UV) while starting material at Rf=0.60. LCMS m/e=352.2(M+H), Rt=4.126 min (Using the method in Step B.)

### Step F. 6-(2,2-Dimethyl-propyl)-4-hydroxy-3,4-dihydro-2H-quinoline-1-carboxylic acid benzyl ester

2.1 mL of 1M (S)-tetrahydro-1-methyl-3,3-diphenyl-1H, 3H-pyrollo[1,2-c][1,3,2] oxazaborole/toluene (2.1 mmol, 0.1 eq.) was added to 7.5 g of the product of Step E (20.8 mmol) in 20 mL of THF cooled to −25° C. The mixture was added dropwise over 20 min via a dropping funnel charged with a solution of 1.4 mL of borane-methylsulfide (14.56 mmol, 0.7 eq.) in 25 mL of THF. The reaction was kept at −20° C., stirred at −20 C. for 1 h, and monitored by TLC.

The reaction was quenched with 50 mL of methanol at −20° C. and allowed to warm to room temperature and stir overnight. The volatiles were removed in vacuo and the residue was purified by flash column to yield 4.4 g of the (R)- alcohol as a light tan oil (60%).

TLC (20% EtOAc/Hexane) Rf=0.18 while starting material at Rf=0.46. LCMS m/e=336.2(M−OH), Rt=3.692 min (Using the method in Step B.)

### Step G. 4-Azido-6-(2,2-dimethyl-propyl)-3,4-dihydro-2H-quinoline-1-carboxylic acid benzyl ester

3.2 mL of diphenylphosphorylazide (DPPA, 14.6 mmol, 1.2 eq.) followed by 2.2 mL of 1,8-diazabicyclo [5.4.0]undec-7-ene (DBU, 14.6 mmol, 1.2 eq.) in 20 mL of toluene were added 4.3 g of the product of Step F (12.2 mmol) in 25 mL of toluene at 0° C. The mixture was allowed to stir at 0° C. for 2 h and room temperature overnight and was monitored by TLC. The mixture was then filtered through a pad of sand-silica gel-sand contained in a Buchner funnel (eluted with 15% EtOAc/Hexane) to remove some precipitates and the volatiles were removed in vacuo to give 3.5 g of the crude S-azide as a white solid (76%). This material was used directly in the next step without further purification.

TLC (20% EtOAc/Hexane) Rf=0.60 (blue color under UV long wave) while starting material at Rf=0.18. LCMS m/e=336.1(M−N₃), Rt=3.404 min (Using the method from Step A.)

### Step I. 4-Amino-6-(2,2-dimethyl-propyl)-3,4-dihydro-2H-quinoline-1-carboxylic acid benzyl ester and 6-(2,2-Dimethyl-propyl)-1-methyl-1,2,3,4-tetrahydro-quinolin-4-ylamine

5.5 mL of 1 M trimethylphosphine/THF at room temperature was added to 2.08 g of the product of Step G (5.5 mmol) in 55 mL of THF and 0.1 mL of water. The mixture was stirred overnight and monitored by TLC. The volatiles were removed in vacuo and the residue was purified by flash column to yield 2.39 g as a light tan oil (75%). TLC (50% EtOAc/Hexane+20% MeOH/DCM, 1:1) Rf=0.35. LCMS m/e=336.1(M−NH₂), Rt=2.472 min (Using the method in Step B.)

Alongside, 0.28 g, a N-methyl tetraquinolin amine, was also isolated as a tan oil. LCMS m/e=216.1(M−NH₂), Rt=0.333 min (Using the method in Step B.)

### Step L. N-{1-(3,5-Difluoro-benzyl)-3-[6-(2,2-dimethyl-propyl)-1-methyl-1,2,3,4-tetrahydro-quinolin-4-ylamino]-2-hydroxy-propyl)-acetamide

LCMS m/e=496.2(M+Na), Rt=2.039 min (Using the method in Step B.). ¹H NMR (CDCl₃) δ 7.56 (s, 1H0, 7.02-6.99 (d, J=8.8 Hz, 1H), 6.89 (m, 1H), 6.74 (m, 1H), 6.68-6.66 (m, 1H), 6.62-6.59 (m, 1H), 4.63 (s, 1H), 4.39-4.32 (m, 1H), 4.12-4.07 (m, 1H), 3.94 (m, 1H), 3.40-3.35 (m, 1H), 3.18-3.15 (m, 1H), 3.05-2.98 (m, 1H), 2.87 (s, 3H), 2.81-2.62 (m, 1H), 2.45-2.37 (m, 1 H), 2.33 (s, 2H),2.32-2.28 (m, 1H), 1.85 (s, 3H), 0.98 (s, 2H), 0.92 (s, 2H), 0.84 (s, 9H). ¹³C NMR (CDCl₃) δ 164.1, 158.9, 133.4, 124.8, 120.8, 111.8, 100.1, 86.7, 83.6, 77.4, 77.0, 76.6, 52.8, 38.3, 31.6, 29.1.

### EXAMPLE 87

### Synthesis of Thiochroman Compounds

### 87.A. (1S,2R)-[3-(6-Bromo-1,1-dioxo-1λ6-thiochroman-4-ylamino)-1-(3,5-difluoro-benzyl)-2-hydroxy-propyl]-carbamic acid tert-butyl ester

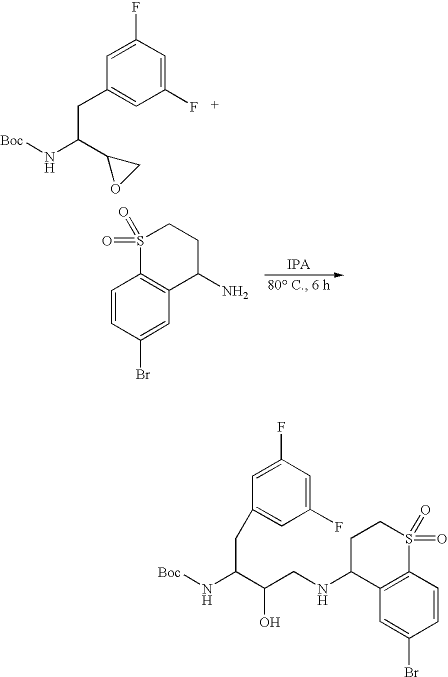

This compound was prepared according to the method described in the examples above, e.g., EXAMPLE 5, Step 8.

HPLC: MH+575.1, retention time=1.8 min, (20-70% Acetonitrile in 1.75 min; 2 mL/min; 35 C; Column=Luna Cl 8(2) 30 cm×4.6 mm).

### 87.B. (1S,2R)-N-[3-(6-Bromo-1,1-dioxo-1λ6-thiochroman-4-ylamino)-1-(3,5-difluoro-benzyl)-2-hydroxy-propyl]-acetamide

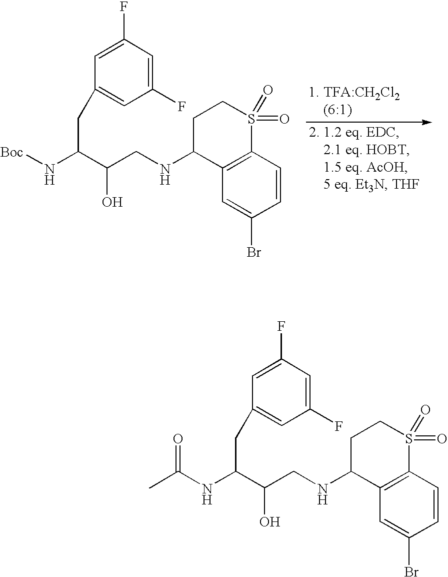

This compound was prepared according to the method described in the examples above, e.g., EXAMPLE 5, step 9.

Diastereomer A: HPLC: MH+517.0, retention time=1.6 min; Diastereomer B: HPLC: MH+517.0, retention time=1.6 min; (20-70% Acetonitrile in 1.75 min; 2 mL/min; 35 C; Column=Luna C18(2) 30 cm×4.6 mm).

### 87.C. (1S,2R)-N-{1-(3,5-Difluoro-benzyl)-3-[6-(2,2-dimethyl-propyl)-2,2-dioxo-2λ6-isothiochroman-4-ylamino]-2-hydroxy-propyl}-acetamide

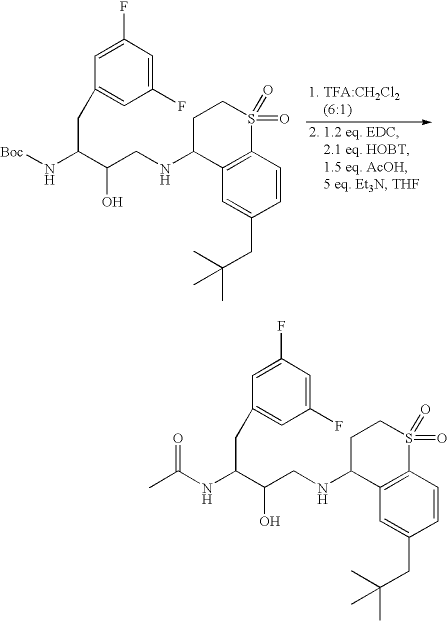

This compound was prepared according to the method described in the examples above, e.g., EXAMPLE 5, step 9.

HPLC: MH+509.2, retention time=2.7 min, (20-70% Acetonitrile in 1.75 min; 2 mL/min; 35 C; Column=Luna C18(2) 30 cm×4.6 mm).

### 87.D. (1S-2R)-N-[1-(3,5-Difluoro-benzyl)-2-hydroxy-3-(6-isobutyl-1,1-dioxo-1λ6-thiochroman-4-ylamino)-propyl]-acetamide

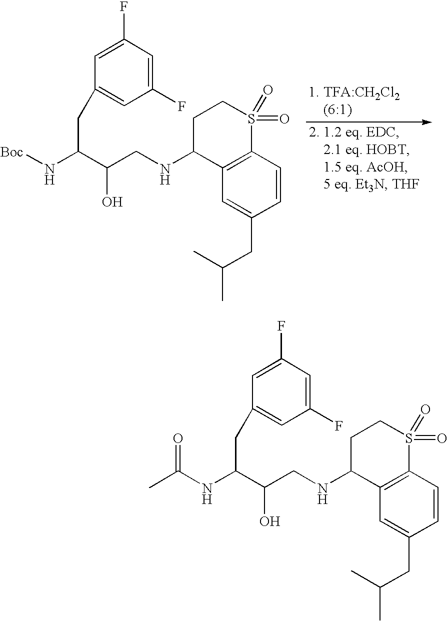

This compound was prepared according to the method described in the examples above, e.g., EXAMPLE 5, step 9.

HPLC: MH+495.2, retention time=1.6 min, (20-70% Acetonitrile in 1.75 min; 2 mL/min; 35 C; Column=Luna Cl 8(2) 30 cm×4.6 mm).

### EXAMPLE 88

### Preparation of Sulfonamides

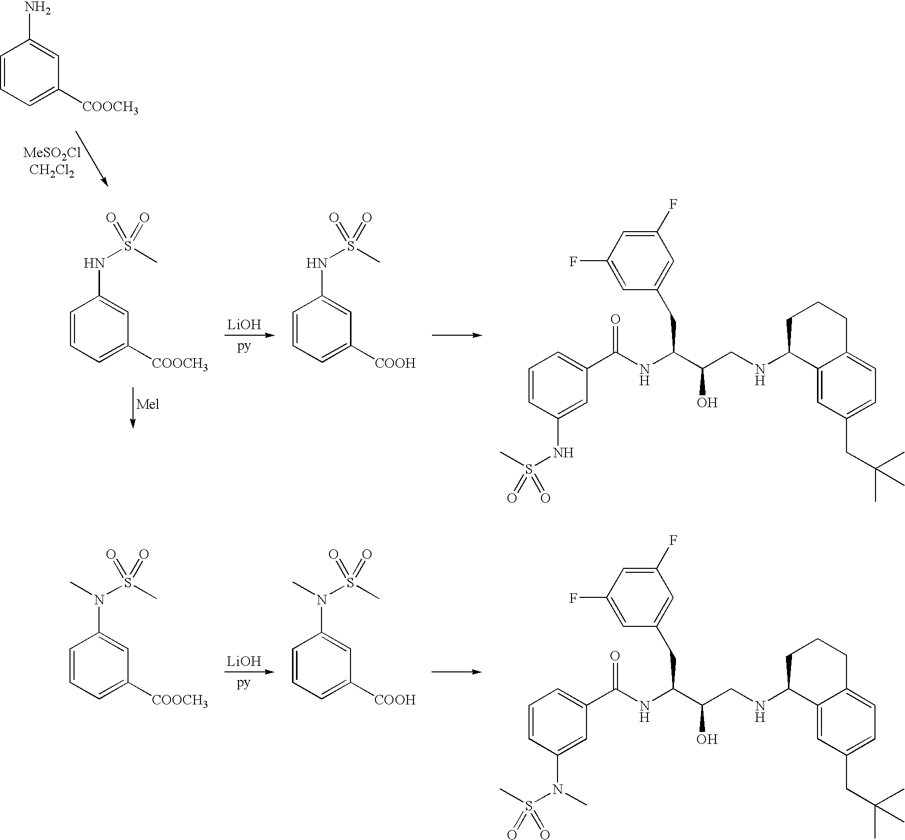

3-(methylsulfonamido)benzoic acid and 3-(N-methylmethylsulfonamido)benzoic acid were synthesized according to the procedure described in WO 2000055153.

### 88.A. Preparation of N-((2S,3R)-1-(3,5-difluorophenyl)-3-hydroxy-4-((S)-7-neopentyl-1,2,3,4-tetra hydronaphthalen-1-ylamino)butan-2-yl)-3-(methylsulfonamido)benzamide

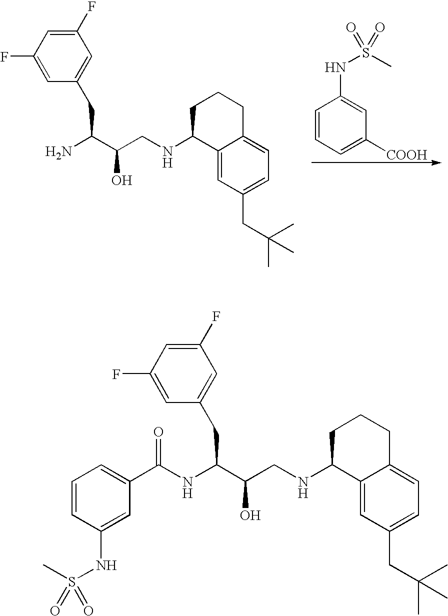

(2R,3S)-3-amino-4-(3,5-difluorophenyl)-1-((S)-7-neopentyl-1,2,3,4-tetrahydronaphthalen-1-ylamino)butan-2-ol, 3-(methylsulfonamido)benzoic acid, and NMM in CH₂Cl₂ were treated with HOBt and EDC at 0° C. The reaction mixture was stirred overnight at room temperature. The solvent was then stripped and the reaction mixture was partitioned between NaHCO₃ and EtOAc. The organic layer was washed with brine, dried, concentrated, and purified by HPLC.

Retention time (min)=2.10, (20-70% Acetonitrile in 1.75 min; 2 ml/min; 35 C; Column=Luna C18(2) 30 cm×4.6 mm); ¹H NMR (300 MHz, CDCl₃) δ 8.21 (bs, 1H), 8.49 (s, 1H), 8.35 (bs, 1H), 7.62 (s, 1H), 7.55-7.41 (m, 3H), 7.33 (d, J=8.1, 1H), 7.15 (s, 1H), 7.07 (s, 1H), 6.75 (d, J=6 Hz, 2H), 6.60 (dt, J=0.9, 2 Hz, 1H), 4.53 (bs, 1H), 4.30-4.22 (m, 2H), 3.22-3.18 (m, 1H), 3.07-2.99 (m, 2H), 2 94 (s, 3H), 2.86-2.76 (m, 3H), 2.64-2.36 (m, 8H), 2.15-1.10 (m, 2H), 1.98-1.80 (m, 2H), 0.86 (s, 9H); MS (ESI) 614.2.

### 88.B. N-((2S,3R)-1-(3,5-difluorophenyl)-3-hydroxy-4-((S)-7-neopentyl-1,2,3,4-tetrahydronaphthalen-1-ylamino)butan-2-yl)-3-(N-methylmethylsulfonamido)benzamide

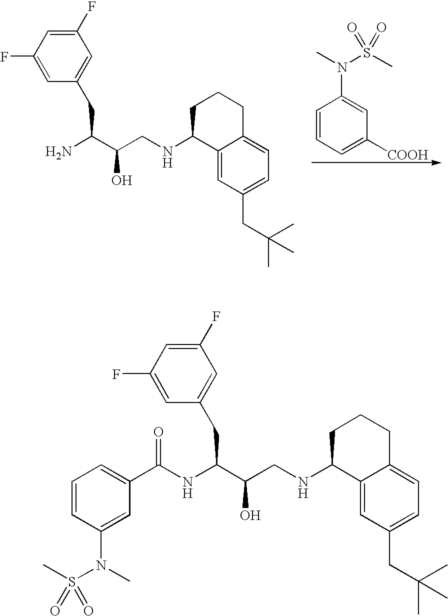

N-((2S,3R)-1-(3,5-difluorophenyl)-3-hydroxy-4-((S)-7-neopentyl-1,2,3,4-tetrahydronaphthalen-1-ylamino)butan-2-yl)-3-(N-methylmethylsulfonamido)benzamide was prepared according to the procedure described in Step A.

Retention time (min)=2.19, (20-70% Acetonitrile in 1.75 min; 2 ml/min; 35 C; Column=Luna C18(2) 30 cm×4.6 mm); ¹H NMR (300 MHz, CDCl₃) δ 8.85 (bs, 1H), 8.45 (bs, 1H), 7.73 (s, 1H), 7.67-7.62 (m, 2H), 7.43 (t, J=7.8 Hz, 1H), 7.10 (d, J=12 Hz, 2H), 7.08 (s,1 H), 6.77 (d, J=6 Hz, 2H), 6.64 (dt, J=9, 2 Hz, 1H), 4.50 (bs, 1 Hz), 4.30-4.15 (m, 2H), 3.32 (s, 3H), 3 22-3.16 (m, 1H), 3.12-3.05 (m, 2H), 2.97-2.77 (m, 4H), 2.83 (s, 3H), 2.45-2.30 (m, 15H), 2.15-2.10 (m, 2H), 2.02-1.85 m, 2H), 0.86 (s, 9H); MS (ESI) 628.3.

### 88.C. N-((2S,3R)-1-(3,5-difluorophenyl)-3-hydroxy-4-((S)-7-neopentyl-1,2,3,4-tetrahydronaphthalen-1-ylamino)butan-2-yl)methanesulfonamide

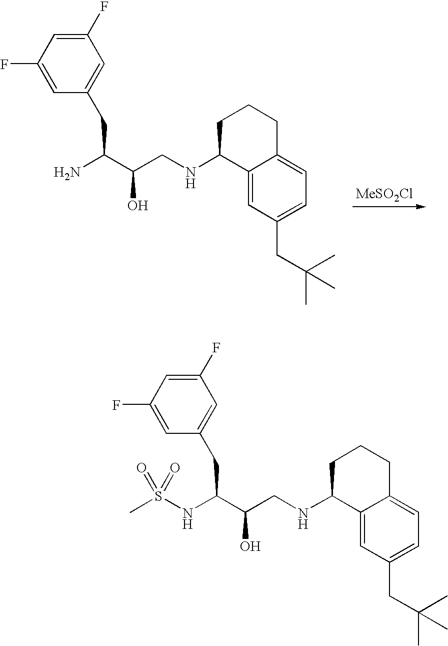

Mesyl chloride (0.015 mL) was added to an ice cold, stirred solution of (2R,3S)-3-amino-4-(3,5-difluorophenyl)-1-((S)-7-neopentyl-1,2,3,4-tetrahydronaphthalen-1-ylamino)butan-2-ol (0.086 g, 0.20 mM) and Et₃N (0.3 mL) in CH₂Cl₂ (4 mL). The reaction mixture was stirred for 30 min and then partitioned between CH₂Cl₂ and water. The organic layer was washed with NaHCO₃, dried, concentrated, and purified by HPLC; yield 0.035 g (36%).

Retention time (min)=2.08, (20-70% Acetonitrile in 1.75 min; 2 ml/min; 35 C; Column=Luna C18(2) 30 cm×4.6 mm); ¹H NMR (300 MHz, CDCl₃) 7.17 (s, 1H), 7.08 (s, 2H), 6.81 (d, J=6 Hz, 2H), 6.72 (dt, J=9, 2 Hz, 1H), 6.09 (d, J=9.6 Hz, 1H), 4.52 (bs, 1H), 4.10 (bs, 1H), 3.58-3.52 (m, 1H), 3.36-3.32 (m, 2H), 3.07-3.02 (m, 2H), 2.92-2.60 (m, 12H), 2.48-2.43 (m, 5H), 2.30 (s, 3H), 2.18-2.12 (m, 3H), 1.99-1.85 (m, 3H), 0.90 (s, 9H); MS (ESI) 495.2.

### EXAMPLE 89

### Examples of Representative Compounds

The following formula (I) compounds can be prepared essentially according to the procedures set forth in the above examples and schemes: Phenyl {1-(3,5-difluorobenzyl)-2-hydroxy-3-[(6-isopropoxy-1,1-dimethyl-3,4-dihydro-1H-isochromen-4-yl)amino]propyl)carbamate, Ethyl 3-[3-(1-{[3-(acetylamino)-4-(3,5-difluorophenyl)-2-hydroxybutyl]amino}cyclohexyl)phenyl]propanoate, N-(1-(3,5-difluorobenzyl)-3-{[7-ethyl-1,2,3,4-tetrahydronaphthalen-1-yl]amino}-2-hydroxypropyl)-2-ethoxyacetamide, N-(1-(3,5-difluorobenzyl)-3-{[(1R)-7-ethyl-1,2,3,4-tetrahydronaphthalen-1-yl]amino}-2-hydroxypropyl)-2,2-difluoroacetamide, 3-(4-{[3-(acetylamino)-4-(3,5-difluorophenyl)-2-hydroxybutyl]amino}-3,4-dihydro-2H-chromen-6-yl)-2-Methylpropanoate, methyl-3-(4-{[3-(acetylamino)-4-(3,5-difluorophenyl)-2-hydroxybutyl]amino}-3,4-dihydro-2H-chromen-6-yl)-2-Methylpropanoate. N-[1-(3,5-Difluoro-benzyl)-3-(6-ethyl-2,2-dioxo-2λ⁶-isothiochromen-4-ylamino)-2-hydroxy-propyl]-2-methyl-2-methylamino-propionamide, {[1-(3,5-Difluoro-benzyl)-3-(6-ethyl-2,2-dioxo-2λ⁶-isothiochromen-4-ylamino)-2-hydroxy-propylcarbamoyl]-methyl}-methyl-carbamic acid tert-butyl ester, N-[1-(3,5-Difluoro-benzyl)-3-(6-ethyl-2,2-dioxo-2λ⁶-isothiochromen-4-ylamino)-2-hydroxy-propyl]-2-phenyl-acetamide, N-[1-(3,5-Difluoro-benzyl)-3-(6-ethyl-2,2-dioxo-2λ⁶-isothiochromen-4-ylamino)-2-hydroxy-propyl]-3-hydroxy-butyramide, N-[1-(3,5-Difluoro-benzyl)-3-(6-ethyl-2,2-dioxo-2λ⁶-isothiochromen-4-ylamino)-2-hydroxy-propyl]-3-hydroxy-propionamide, N-[1-(3,5-Difluoro-benzyl)-3-(6-ethyl-2,2-dioxo-2λ⁶-isothiochromen-4-ylamino)-2-hydroxy-propyl]-3-hydroxy-2,2-dimethyl-propionamide, N-[1-(3,5-Difluoro-benzyl)-3-(6-ethyl-2,2-dioxo-2λ⁶-isothiochromen-4-ylamino)-2-hydroxy-propyl]-3-methyl-butyramide, N-[1-(3,5-Difluoro-benzyl)-3-(6-ethyl-2,2-dioxo-2λ⁶-isothiochromen-4-ylamino)-2-hydroxy-propyl]-2-methylamino-acetamide, N-[1-(3,5-Difluoro-benzyl)-3-(6-ethyl-2,2-dioxo-2λ⁶-isothiochromen-4-ylamino)-2-hydroxy-propyl]-3-methyl-butyramide, N-[1-(3,5-Difluoro-benzyl)-3-(6-ethyl-2,2-dioxo-2λ⁶-isothiochromen-4-ylamino)-2-hydroxy-propyl]-3-hydroxy-2,2-dimethyl-propionamide, N-[1-(3,5-Difluoro-benzyl)-3-(6-ethyl-2,2-dioxo-2λ⁶-isothiochromen-4-ylamino)-2-hydroxy-propyl]-3-hydroxy-propionamide, N-[1-(3,5-Difluoro-benzyl)-3-(6-ethyl-2,2-dioxo-2λ⁶-isothiochromen-4-ylamino)-2-hydroxy-propyl]-3-hydroxy-butyramide, N-[1-(3,5-Difluoro-benzyl)-3-(6-ethyl-2,2-dioxo-2λ⁶-isothiochromen-4-ylamino)-2-hydroxy-propyl]-3-methyl-butyramide, N-[1-(3,5-Difluoro-benzyl)-3-(6-ethyl-2,2-dioxo-2λ⁶-isothiochromen-4-ylamino)-2-hydroxy-propyl]-3-hydroxy-2,2-dimethyl-propionamide, N-[l-(3,5-Difluoro-benzyl)-3-(6-ethyl-2,2-dioxo-2λ⁶-isothiochromen-4-ylamino)-2-hydroxy-propyl]-3-hydroxy-propionamide, N-[1-(3,5-Difluoro-benzyl)-3-(6-ethyl-2,2-dioxo-2λ⁶-isothiochromen-4-ylamino)-2-hydroxy-propyl]-3-hydroxy-butyramide, {[1-(3,5-Difluoro-benzyl)-3-(6-ethyl-2,2-dioxo-2λ⁶-isothiochromen-4-ylamino)-2-hydroxy-propylcarbamoyl]-methyl}-methyl-carbamic acid tert-butyl ester, N-[1-(3,5-Difluoro-benzyl)-3-(6-ethyl-2,2-dioxo-2λ⁶-isothiochromen-4-ylamino)-2-hydroxy-propyl]-2-methyl-2-methylamino-propionamide, N-[1-(3,5-Difluoro-benzyl)-3-(6-ethyl-2,2-dioxo-2λ⁶-isothiochromen-4-ylamino)-2-hydroxy-propyl]-propionamide, {[1-(3,5-Difluoro-benzyl)-3-(6-ethyl-2,2-dioxo-2-isothiochromen-4-ylamino)-2-hydroxy-propylcarbamoyl]-methyl}-methyl-carbamic acid tert-butyl ester, N-[1-(3,5-Difluoro-benzyl)-3-(6-ethyl-2,2-dioxo-2λ⁶-isothiochromen-4-ylamino)-2-hydroxy-propyl]-2-methyl-2-methylamino-propionamide, N-[1-(3,5-Difluoro-benzyl)-3-(6-ethyl-2,2-dioxo-2λ⁶-isothiochromen-4-ylamino)-2-hydroxy-propyl]-propionamide, N-[1-(3,5-Difluoro-benzyl)-3-(7-ethyl-1,2,3,4-tetrahydro-naphthalen-1-ylamino)-2-hydroxy-propyl]-2-ethoxy-acetamide, N-(1-(3,5-difluorobenzyl)-3-{[7-ethyl-1,2,3,4-tetrahydronaphthalen-1-yl]amino}-2-hydroxypropyl)-2,2-difluoroacetamide, N-[1-(3,5-Difluorobenzyl)-3-(7-ethyl-1,2,3,4-tetrahydro-naphthalen-1-ylamino)-2-hydroxypropyl]-2-hydroxy-acetamide, N-[1-(3,5-Difluorobenzyl)-3-(7-ethyl-1,2,3,4-tetrahydro-naphthalen-1-ylamino)-2-hydroxy-propyl]-2-methoxy-acetamide, N-[1-(3,5-Difluorobenzyl)-3-(7-ethyl-1,2,3,4-tetrahydro-naphthalen-1-ylamino)-2-hydroxy-propyl]-propionamide, 2-(2-Butoxy-ethoxy)-N-[1-(3,5-difluorobenzyl)-3-(7-ethyl-1,2,3,4-tetrahydro-naphthalen-1-ylamino)-2-hydroxy-propyl]-acetamide, 5-Oxo-hexanoic acid [1-(3,5-difluorobenzyl)-3-(7-ethyl-1,2,3,4-tetrahydro-naphthalen-1-ylamino)-2-hydroxy-propyl]-amide, N-[1-(3,5-Difluorobenzyl)-3-(7-ethyl-1,2,3,4-tetrahydro-naphthalen-1-ylamino)-2-hydroxy-propyl]-N′,N′-dimethyl-succinamide, Pentanoic acid [1-(3,5-difluoro-benzyl)-3-(7-ethyl-1,2,3,4-tetrahydro-naphthalen-1-ylamino)-2-hydroxy-propyl]-amide, N-[1-(3,5-Difluorobenzyl)-3-(7-ethyl-1,2,3,4-tetrahydro-naphthalen-1-ylamino)-2-hydroxy-propyl]-2-(2-oxo-cyclopentyl)-acetamide, Pent-3-enoic acid [1-(3,5-difluoro-benzyl)-3-(7-ethyl-1,2,3,4-tetrahydro-naphthalen-1-ylamino)-2-hydroxy-propyl]-amide, Hex-3-enoic acid [1-(3,5-difluoro-benzyl)-3-(7-ethyl-1,2,3,4-tetrahydro-naphthalen-1-ylamino)-2-hydroxy-propyl]-amide, 3-Allyloxy-N-[1-(3,5-difluoro-benzyl)-3-(7-ethyl-1,2,3,4-tetrahydro-naphthalen-1-ylamino)-2-hydroxy-propyl]-propionamide, 2,2-Dichloro-N-[1-(3,5-difluoro-benzyl)-3-(7-ethyl-1,2,3,4-tetrahydro-naphthalen-1-ylamino)-2-hydroxy-propyl]-acetamide, 2-Chloro-N-[1-(3,5-difluoro-benzyl)-3-(7-ethyl-1,2,3,4-tetrahydro-naphthalen-1-ylamino)-2-hydroxy-propyl]-acetamide, 2-Bromo-N-[1-(3,5-difluoro-benzyl)-3-(7-ethyl-1,2,3,4-tetrahydro-naphthalen-1-ylamino)-2-hydroxy-propyl]-acetamide, N-(1-(3,5-difluorobenzyl)-3-{[7-ethyl-1,2,3,4-tetrahydronaphthalen-1-yl]amino}-2-hydroxypropyl)ethanethioamide hydrochloride, N-[1-(3,5-Difluorobenzyl)-3-(7-ethyl-1,2,3,4-tetrahydro-naphthalen-1-ylamino)-2-hydroxy-propyl]-methanesulfonamide, tert-butyl 1-(3,5-difluorobenzyl)-3-[(6-ethyl-1-methyl-1,2,3,4-tetrahydroquinolin-4-yl)amino]-2-hydroxypropylcarbamate, N-(1-(3,5-difluorobenzyl)-3-{[7-(2,2-dimethylpropyl)-1,2,3,4-tetrahydro naphthalen-1-yl]amino}-2-hydroxypropyl)-2-fluoroacetamide, N-(1-(3,5-difluorobenzyl)-3-{[7-ethyl-1,2,3,4-tetrahydronaphthalen-1-yl]amino}-2-hydroxypropyl)-2-ethoxyacetamide, N-[1-(3,5-Difluoro-benzyl)-3-(6-ethyl-2,2-dioxo-2λ⁶-isothiochromen-4-ylamino)-2-hydroxy-propyl]-propionamide, N-[1-(3,5-Difluoro-benzyl)-3-(6-ethyl-2,2-dioxo-2A⁶-isothiochromen-4-ylamino)-2-hydroxy-propyl]-2-(1H-imidazol-4-yl)-acetamide, N-[1-(3,5-Difluoro-benzyl)-3-(6-ethyl-2,2-dioxo-2λ⁶-isothiochromen-4-ylamino)-2-hydroxy-propyl]-2-methyl-2-methylamino-propionamide, {[1-(3,5-Difluoro-benzyl)-3-(6-ethyl-2,2-dioxo-2λ⁶-isothiochromen-4-ylamino)-2-hydroxy-propylcarbamoyl]-methyl}-methyl-carbamic acid tert-butyl ester, N-[1-(3,5-Difluoro-benzyl)-3-(6-ethyl-2,2-dioxo-2λ⁶-isothiochromen-4-ylamino)-2-hydroxy-propyl]-2-phenyl-acetamide, N-[1-(3,5-Difluoro-benzyl)-3-(6-ethyl-2,2-dioxo-2λ⁶-isothiochromen-4-ylamino)-2-hydroxy-propyl]-3-hydroxy-butyramide, N-[1-(3,5-Difluoro-benzyl)-3-(6-ethyl-2,2-dioxo-2λ⁶-isothiochromen-4-ylamino)-2-hydroxy-propyl]-3-hydroxy-propionamide, N-[1-(3,5-Difluoro-benzyl)-3-(6-ethyl-2,2-dioxo-2λ⁶-isothiochromen-4-ylamino)-2-hydroxy-propyl]-3-hydroxy-2,2-dimethyl-propionamide, N-[1-(3,5-Difluoro-benzyl)-3-(6-ethyl-2,2-dioxo-2λ⁶-isothiochromen-4-ylamino)-2-hydroxy-propyl]-3-methyl-butyramide, 2-Amino-N-[1-(3,5-difluoro-benzyl)-3-(6-ethyl-2,2-dioxo-2λ⁶-isothiochromen-4-ylamino)-2-hydroxy-propyl]-acetamide, N-[1-(3,5-Difluoro-benzyl)-3-(6-ethyl-2,2-dioxo-2λ⁶-isothiochromen-4-ylamino)-2-hydroxy-propyl]-2-methylamino-acetamide, and N-(1-(3,5-difluorobenzyl)-3-{[7-ethyl-1,2,3,4-tetrahydronaphthalen-1-yl]amino)-2-hydroxypropyl)-2,2-difluoroacetamide, or pharmaceutical salts thereof.

Generally, the protection of amines is conducted, where appropriate, by methods known to those skilled in the art. See, for example, Protecting Groups in Organic Synthesis, John Wiley and sons, New York, N.Y., 1981, Chapter 7; Protecting Groups in Organic Chemistry, Plenum Press, New York, N.Y., 1973, Chapter 2. When the amino protecting group is no longer needed, it is removed by methods known to those skilled in the art. By definition the amino protecting group must be readily removable. A variety of suitable methodologies are known to those skilled in the art; see also T. W. Green and P. G. M. Wuts in Protective Groups in Organic Chemistry, John Wiley and Sons, 3^{rd} edition, 1999. Suitable amino protecting groups include t-butoxycarbonyl, benzyl-oxycarbonyl, formyl, trityl, phthalimido, trichloro-acetyl, chloroacetyl, bromoacetyl, iodoacetyl, 4-phenylbenzyloxycarbonyl, 2-methylbenzyloxycarbonyl, 4-ethoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, 2-(4-xenyl)isopropoxycarbonyl, 1,1-diphenyleth-1-yloxycarbonyl, 1,1-diphenylprop-1-yloxycarbonyl, 2-phenylprop-2-yloxycarbonyl, 2-(p-toluyl)prop-2-yloxy-carbonyl, cyclopentanyloxycarbonyl, 1-methylcyclopentanyloxycarbonyl, cyclohexanyloxycarbonyl, 1-methyl-cyclohexanyloxycabonyl, 2-methylcyclohexanyloxycarbonyl, 2-(4-toluylsulfonyl)ethoxycarbonyl, 2-(methylsulfonyl)-ethoxycarbonyl, 2-(triphenylphosphino)ethoxycarbonyl, fluorenylmethoxycarbonyl, 2-(trimethylsilyl)ethoxy-carbonyl, allyloxycarbonyl, 1-(trimethylsilylmethyl)prop-1-enyloxycarbonyl, 5-benzisoxalylmethoxycarbonyl, 4-acetoxybenzyloxycarbonyl, 2,2,2-trichloroethoxycarbonyl, 2-ethynyl-2-propoxycarbonyl, cyclopropylmethoxycarbonyl, 4-(decyloxyl)benzyloxycarbonyl, isobornyloxycarbonyl, 1-piperidyloxycarbonyl, 9-fluoroenylmethyl carbonate, —CH—CH═CH₂ and the like.

In an embodiment, the protecting group is t-butoxycarbonyl (Boc) and/or benzyloxycarbonyl (CBZ). In another embodiment, the protecting group is Boc. One skilled in the art will recognize suitable methods of introducing a Boc or CBZ protecting group and may additionally consult Protective Groups in Organic Chemistry for guidance.

The compounds of the present invention may contain geometric or optical isomers as tautomers. Thus, the present invention includes all tautomers and pure geometric isomers, such as the E and Z geometric isomers, as mixtures thereof. Further, the present invention includes pure enantiomers, diastereomers and/or mixtures thereof, including racemic mixtures. The individual geometric isomers, enantiomers or diastereomers may be prepared or isolated by methods known to those in the art, including, for example chiral chromatography, preparing diastereomers, separating the diastereomers and then converting the diastereomers into enantiomers.

Compounds of the present invention with designated stereochemistry can be included in mixtures, including racemic mixtures, with other enantiomers, diastereomers, geometric isomers or tautomers. In a preferred embodiment, compounds of the present invention are typically present in these mixtures in diastereomeric and/or enantiomeric excess of at least 50%. Preferably, compounds of the present invention are present in these mixtures in diastereomeric and/or enantiomeric excess of at least 80%. More preferably, compounds of the present invention with the desired stereochemistry are present in diastereomeric and/or enantiomeric excess of at least 90%. Even more preferably, compounds of the present invention with the desired stereochemistry are present in diastereomeric and/or enantiomeric excess of at least 99%. Preferably the compounds of the present invention have the “S” configuration at position 1. Also preferred are compounds that have the “R” configuration at position 2. Most preferred are compounds that have the “1S,2R” configuration.

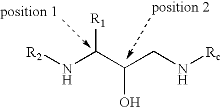

All compound names were generated using AutoNom (AUTOmatic NOMenclature) version 2.1, ACD Namepro version 5.09, Chemdraw Ultra (versions 6.0, 8.0, 8.03, and 9.0), or were derived therefrom.

Several of the compounds of formula (I) are amines, and as such form salts when reacted with acids. Pharmaceutically acceptable salts are preferred over the corresponding amines since they produce compounds that are more water soluble, stable and/or more crystalline.

### EXAMPLE 90

### Exemplary Formula (I) Compounds

| Example No. | Comppound |
|---|---|
| 90.1. | 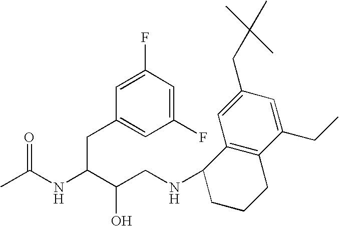 |
| | N-{1-(3,5-Difluoro-benzyl)-3-[7-(2,2-dimethyl-propyl)-5-ethyl- |
| | 1,2,3,4-tetrahydro-naphthalen-1-ylamino]-2-hydroxy-propyl}- |
| | acetamide |
| 90.2. | 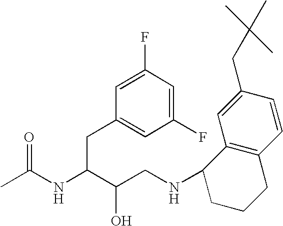 |
| | N-{1-(3,5-Difluoro-benzyl)-3-[7-(2,2-dimethyl-propyl)-1,2,3,4- |
| | tetrahydro-naphthalen-1-ylamino]-2-hydroxy-propyl}-acetamide |
| 90.3. | 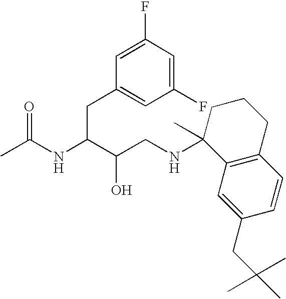 |
| | N-{1-(3,5-Difluoro-benzyl)-3-[7-(2,2-dimethyl-propyl)-1-methyl- |
| | 1,2,3,4-tetrahydro-naphthalen-1-ylamino]-2-hydroxy-propyl}- |
| | acetamide |
| 90.4. | 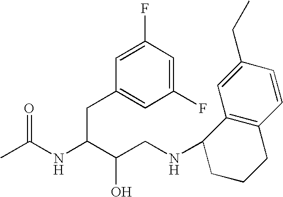 |
| | N-[1-(3,5-Difluoro-benzyl)-3-(7-ethyl-1,2,3,4-tetrahydro-naphthalen- |
| | 1-ylamino)-2-hydroxy-propyl]-acetamide |
| 90.5. | 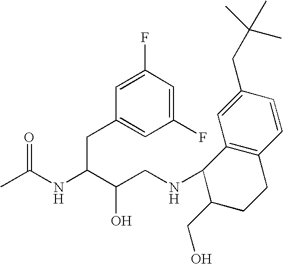 |
| | N-{1-(3,5-Difluoro-benzyl)-3-[7-(2,2-dimethyl-propyl)-2- |
| | hydroxymethyl-1,2,3,4-tetrahydro-naphthalen-1-ylamino]-2- |
| | hydroxy-propyl}-acetamide |
| 90.6. | 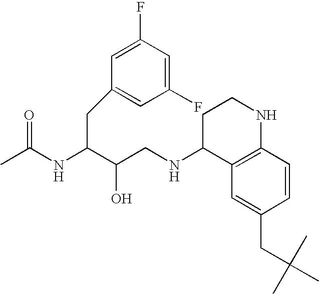 |
| | N-{1-(3,5-Difluoro-benzyl)-3-[6-(2,2-dimethyl-propyl)-1,2,3,4- |
| | tetrahydro-quinolin-4-ylamino]-2-hydroxy-propyl}-acetamide |
| 90.7. | 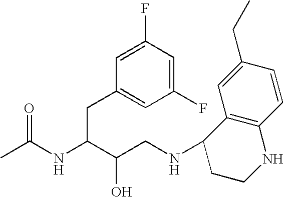 |
| | N-[1-(3,5-Difluoro-benzyl)-3-(6-ethyl-1,2,3,4-tetrahydro-quinolin-4- |
| | ylamino)-2-hydroxy-propyl]-acetamide |
| 90.8. | 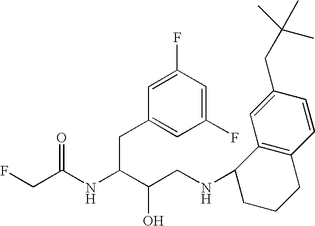 |
| | N-{1-(3,5-Difluoro-benzyl)-3-[7-(2,2-dimethyl-propyl)-1,2,3,4- |
| | tetrahydro-naphthalen-1-ylamino]-2-hydroxy-propyl}-2-fluoro- |
| | acetamide |
| 90.9. | 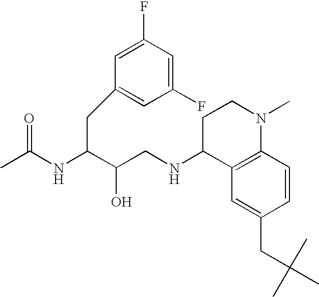 |
| | N-{1-(3,5-Difluoro-benzyl)-3-[6-(2,2-dimethyl-propyl)-1-methyl- |
| | 1,2,3,4-tetrahydro-quinolin-4-ylamino]-2-hydroxy-propyl}-acetamide |
| 90.10. | 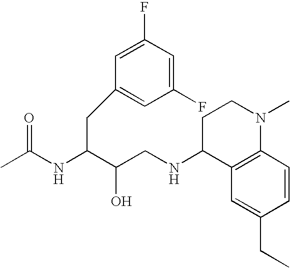 |
| | N-[1-(3,5-Difluoro-benzyl)-3-(6-ethyl-1-methyl-1,2,3,4-tetrahydro- |
| | quinolin-4-ylamino)-2-hydroxy-propyl]-acetamide |
| 90.11. | 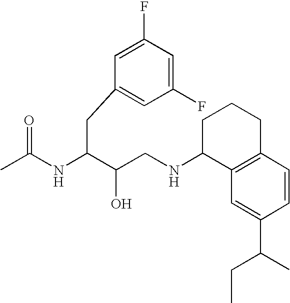 |
| | N-[3-(7-sec-Butyl-1,2,3,4-tetrahydro-naphthalen-1-ylamino)-1-(3,5- |
| | difluoro-benzyl)-2-hydroxy-propyl]-acetamide |
| 90.12. | 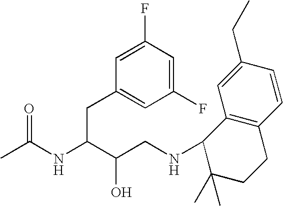 |
| | N-[1-(3,5-Difluoro-benzyl)-3-(7-ethyl-2,2-dimethyl-1,2,3,4- |
| | tetrahydro-naphthalen-1-ylamino)-2-hydroxy-propyl]-acetamide |
| 90.13. | 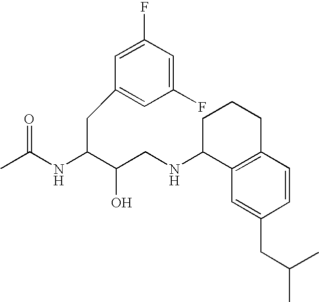 |
| | N-[1-(3,5-Difluoro-benzyl)-2-hydroxy-3-(7-isobutyl-1,2,3,4- |
| | tetrahydro-naphthalen-1-ylamino)-propyl]-acetamide |
| 90.14. | 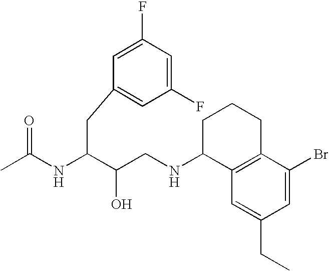 |
| | N-[3-(5-Bromo-7-ethyl-1,2,3,4-tetrahydro-naphthalen-1-ylamino)-1- |
| | (3,5-difluoro-benzyl)-2-hydroxy-propyl]-acetamide |
| 90.15. | 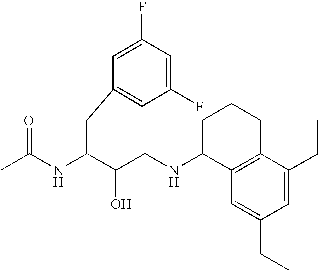 |
| | N-[3-(5,7-Diethyl-1,2,3,4-tetrahydro-naphthalen-1-ylamino)-1-(3,5- |
| | difluoro-benzyl)-2-hydroxy-propyl]-acetamide |
| 90.16. | 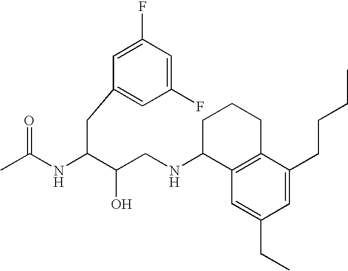 |
| | N-[3-(5-Butyl-7-ethyl-1,2,3,4-tetrahydro-naphthalen-1-ylamino)-1- |
| | (3,5-difluoro-benzyl)-2-hydroxy-propyl]-acetamide |
| 90.17. | 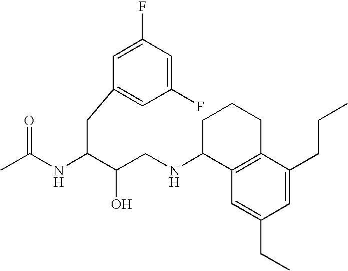 |
| | N-[1-(3,5-Difluoro-benzyl)-3-(7-ethyl-5-propyl-1,2,3,4-tetrahydro- |
| | naphthalen-1-ylamino)-2-hydroxy-propyl]-acetamide |
| 90.18. | 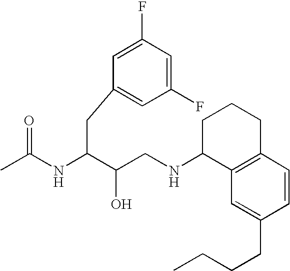 |
| | N-[3-(7-Butyl-1,2,3,4-tetrahydro-naphthalen-1-ylamino)-1-(3,5- |
| | difluoro-benzyl)-2-hydroxy-propyl]-acetamide |
| 90.19. | 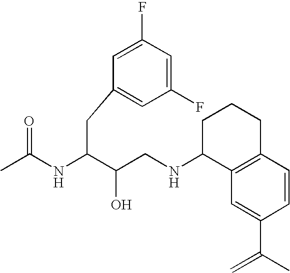 |
| | N-[1-(3,5-Difluoro-benzyl)-2-hydroxy-3-(7-isopropenyl-1,2,3,4- |
| | tetrahydro-naphthalen-1-ylamino)-propyl]-acetamide |
| 90.20. | 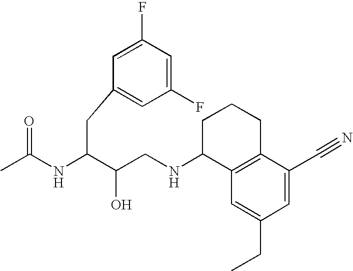 |
| | N-[3-(5-Cyano-7-ethyl-1,2,3,4-tetrahydro-naphthalen-1-ylamino)-1- |
| | (3,5-difluoro-benzyl)-2-hydroxy-propyl]-acetamide |
| 90.21. | 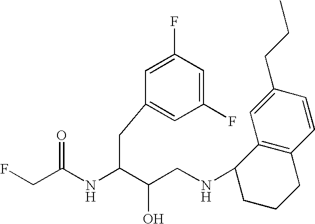 |
| | N-[1-(3,5-Difluoro-benzyl)-2-hydroxy-3-(7-propyl-1,2,3,4-tetrahydro- |
| | naphthalen-1-ylamino)-propyl]-2-fluoro-acetamide |
| 90.22. | 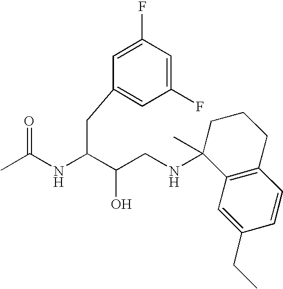 |
| | N-[1-(3,5-Difluoro-benzyl)-2-hydroxy-3-(1-methyl-7-ethyl-1,2,3,4- |
| | tetrahydro-naphthalen-1-ylamino)-propyl]-acetamide |
| 90.23. | 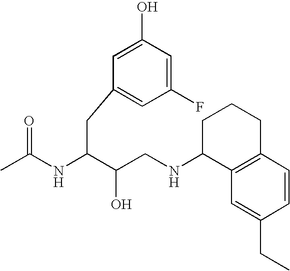 |
| | N-[3-(7-Ethyl-1,2,3,4-tetrahydro-naphthalen-1-ylamino)-1-(3-fluoro- |
| | 5-hydroxy-benzyl)-2-hydroxy-propyl]-acetamide |
| 90.24. | 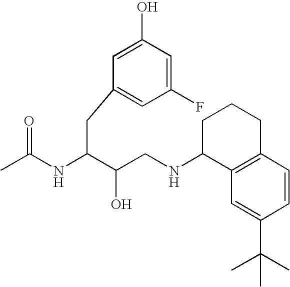 |
| | N-[3-(7-tert-Butyl-1,2,3,4-tetrahydro-naphthalen-1-ylamino)-1-(3- |
| | fluoro-5-hydroxy-benzyl)-2-hydroxy-propyl]-acetamide |
| 90.25. | 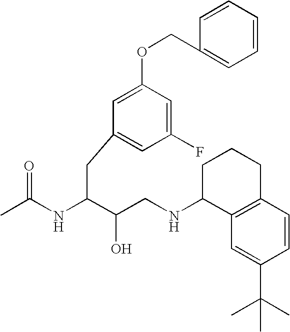 |
| | N-[1-(3-Benzyloxy-5-fluoro-benzyl)-3-(7-tert-butyl-1,2,3,4- |
| | tetrahydro-naphthalen-1-ylamino)-2-hydroxy-propyl]-acetamide |
| 90.26. | 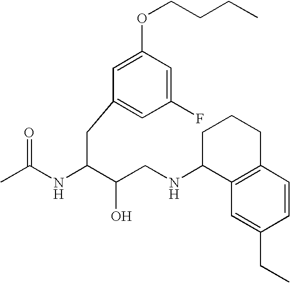 |
| | N-[1-(3-Butoxy-5-fluoro-benzyl)-3-(7-ethyl-1,2,3,4-tetrahydro- |
| | naphthalen-1-ylamino)-2-hydroxy-propyl]-acetamide |
| 90.27. | 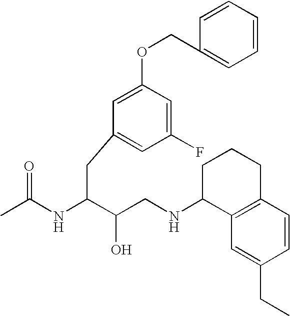 |
| | N-[1-(3-Benzyloxy-5-fluoro-benzyl)-3-(7-ethyl-1,2,3,4-tetrahydro- |
| | naphthalen-1-ylamino)-2-hydroxy-propyl]-acetamide |
| 90.28. | 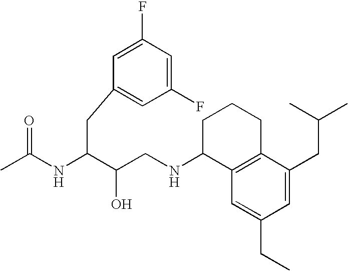 |
| | N-[1-(3,5-Difluoro-benzyl)-3-(7-ethyl-5-isobutyl-1,2,3,4-tetrahydro- |
| | naphthalen-1-ylamino)-2-hydroxy-propyl]-acetamide |
| 90.29. | 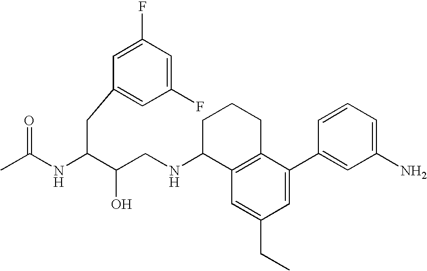 |
| | N-[3-[5-(3-Amino-phenyl)-7-ethyl-1,2,3,4-tetrahydro-naphthalen-1- |
| | ylamino]-1-(3,5-difluoro-benzyl)-2-hydroxy-propyl]-acetamide |
| 90.30. | 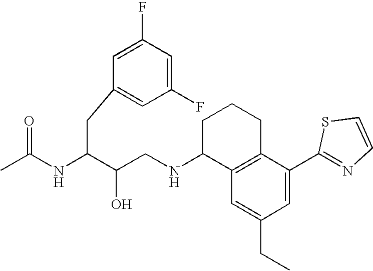 |
| | N-[1-(3,5-Difluoro-benzyl)-3-(7-ethyl-5-thiazol-2-yl-1,2,3,4- |
| | tetrahydro-naphthalen-1-ylamino)-2-hydroxy-propyl]-acetamide |
| 90.31. | 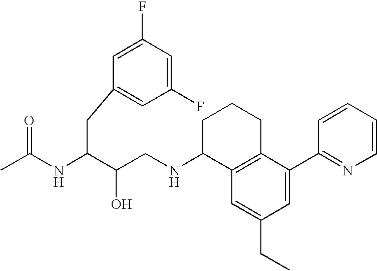 |
| | N-[1-(3,5-Difluoro-benzyl)-3-(7-ethyl-5-pyridin-2-yl-1,2,3,4- |
| | tetrahydro-naphthalen-1-ylamino)-2-hydroxy-propyl]-acetamide |
| 90.32. | 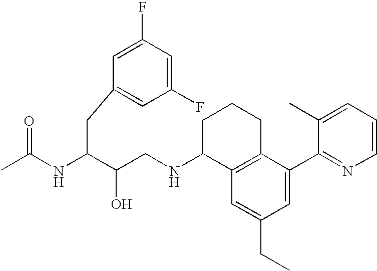 |
| | N-{1-(3,5-Difluoro-benzyl)-3-[7-ethyl-5-(3-methyl-pyridin-2-yl)- |
| | 1,2,3,4-tetrahydro-naphthalen-1-ylamino]-2-hydroxy-propyl)- |
| | acetamide |
| 90.33. | 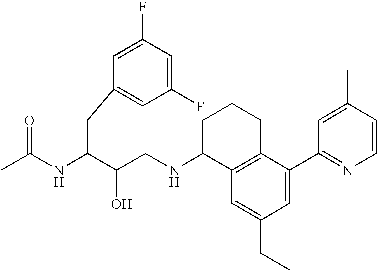 |
| | N-{1-(3,5-Difluoro-benzyl)-3-[7-ethyl-5-(4-methyl-pyridin-2-yl)- |
| | 1,2,3,4-tetrahydro-naphthalen-1-ylamino]-2-hydroxy-propyl}- |
| | acetamide |
| 90.34. | 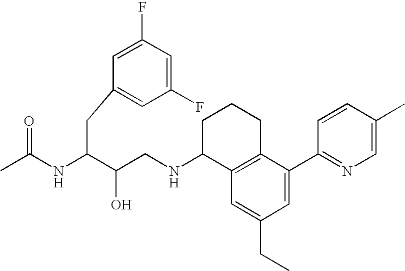 |
| | N-{1-(3,5-Difluoro-benzyl)-3-[7-ethyl-5-(5-methyl-pyridin-2-yl)- |
| | 1,2,3,4-tetrahydro-naphthalen-1-ylamino]-2-hydroxy-propyl}- |
| | acetamide |
| 90.35. | 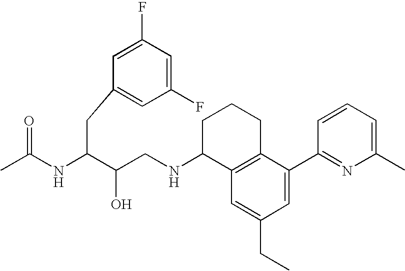 |
| | N-{1-(3,5-Difluoro-benzyl)-3-[7-ethyl-5-(6-methyl-pyridin-2-yl)- |
| | 1,2,3,4-tetrahydro-naphthalen-1-ylamino]-2-hydroxy-propyl}- |
| | acetamide |
| 90.36. | 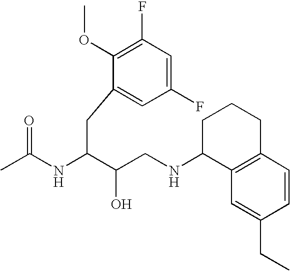 |
| | N-[1-(3,5-Difluoro-2-methoxy-benzyl)-3-(7-ethyl-1,2,3,4-tetrahydro- |
| | naphthalen-1-ylamino)-2-hydroxy-propyl]-acetamide |
| 90.37. | 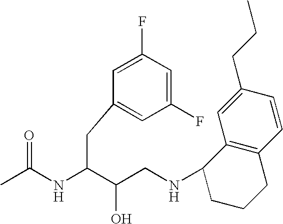 |
| | N-(1-(3,5-difluorophenyl)-3-hydroxy-4-(7-propyl-1,2,3,4- |
| | tetrahydronaphthalen-1-ylamino)butan-2-yl)acetamide |
| 90.38. | 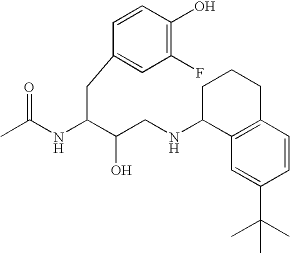 |
| | N-(4-(7-tert-butyl-1,2,3,4- |
| | tetrahydronaphthalen-1-ylamino)-1-(3-fluoro-4-hydroxyphenyl)-3- |
| | hydroxybutan-2-yl)acetamide |
| 90.39. | 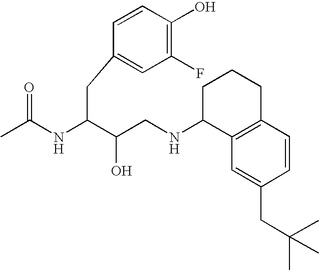 |
| | N-(1-(3-fluoro-4-hydroxyphenyl)-3-hydroxy-4-(7-neopentyl-1,2,3,4- |
| | tetrahydronaphthalen-1-ylamino)butan-2-yl)acetamide |
| 90.40. | 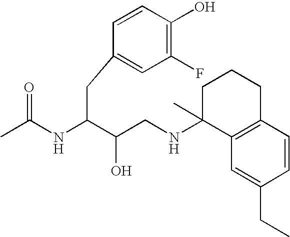 |
| | N-(4-(7-ethyl-1-methyl-1,2,3,4-tetrahydronaphthalen-1-ylamino)-1- |
| | (3-fluoro-4-hydroxyphenyl)-3-hydroxybutan-2-yl)acetamide |
| 90.41. | 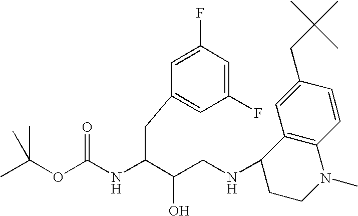 |
| | {1-(3,5-Difluoro-benzyl)-3-[6-(2,2-dimethyl-propyl)-1-methyl- |
| | 1,2,3,4-tetrahydro-quinolin-4-ylamino]-2-hydroxy-propyl}-carbamic |
| | acid tert-butyl ester |
| 90.42. | 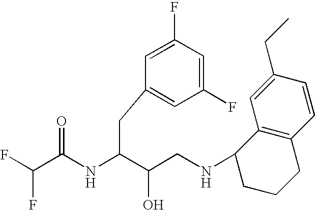 |
| | N-[1-(3,5-Difluoro-benzyl)-3-(7-ethyl-1,2,3,4-tetrahydro-naphthalen- |
| | 1-ylamino)-2-hydroxy-propyl]-2,2-difluoro-acetamide |
| 90.43. | 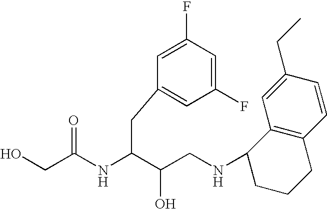 |
| | N-[1-(3,5-Difluoro-benzyl)-3-(7-ethyl-1,2,3,4-tetrahydro-naphthalen- |
| | 1-ylamino)-2-hydroxy-propyl]-2-hydroxy-acetamide |
| 90.44. | 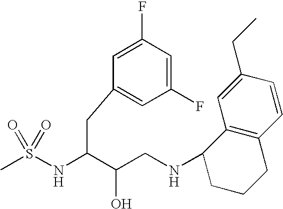 |
| | N-(1-(3,5-difluorophenyl)-4-(7-ethyl-1,2,3,4-tetrahydronaphthalen- |
| | 1-ylamino)-3-hydroxybutan-2-yl)methanesulfonamide |
| 90.45. | 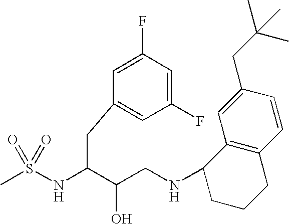 |
| | N-(1-(3,5-difluorophenyl)-3-hydroxy-4-(7-neopentyl-1,2,3,4- |
| | tetrahydronaphthalen-1-ylamino)butan-2-yl)methanesulfonamide |
| 90.46. | 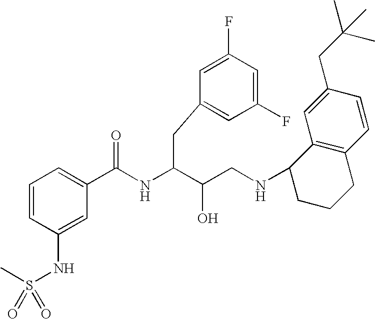 |
| | N-(1-(3,5-difluorophenyl)-3-hydroxy-4-(7-neopentyl-1,2,3,4- |
| | tetrahydronaphthalen-1-ylamino)butan-2-yl)-3- |
| | (methylsulfonamido)benzamide |
| 90.47. | 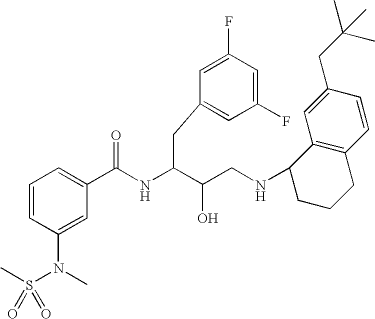 |
| | N-(1-(3,5-difluorophenyl)-3-hydroxy-4-(7-neopentyl-1,2,3,4- |
| | tetrahydronaphthalen-1-ylamino)butan-2-yl)-3-(N- |
| | methylmethylsulfonamido)benzamide |

### EXAMPLE 91

### Biological Examples

Properties such as efficacy, oral bioavailability, selectivity, or blood-brain barrier penetration can be assessed by techniques and assays known to one skilled in the art. Exemplary assays for determining such properties are found below.

### Inhibition of APP Cleavage

The methods of treatment and compounds of the present invention inhibit cleavage of APP between Met595 and Asp596 numbered for the APP695 isoform, or a mutant thereof, or at a corresponding site of a different isoform, such as APP751 or APP770, or a mutant thereof (sometimes referred to as the “beta secretase site”.). While many theories exist, inhibition of beta-secretase activity is thought to inhibit production of A-beta.

Inhibitory activity is demonstrated in one of a variety of inhibition assays, whereby cleavage of an APP substrate in the presence of beta-secretase enzyme is analyzed in the presence of the inhibitory compound, under conditions normally sufficient to result in cleavage at the beta-secretase cleavage site. Reduction of APP cleavage at the beta-secretase cleavage site compared with an untreated or inactive control is correlated with inhibitory activity. Assay systems that can be used to demonstrate efficacy of the compounds of formula (I) are known. Representative assay systems are described, for example, in U.S. Pat. Nos. 5,942,400 and 5,744,346, as well as in the Examples below.

The enzymatic activity of beta-secretase and the production of A-beta can be analyzed in vitro or in vivo, using natural, mutated, and/or synthetic APP substrates, natural, mutated, and/or synthetic enzyme, and the compound employed in the particular method of treatment. The analysis can involve primary or secondary cells expressing native, mutant, and/or synthetic APP and enzyme, animal models expressing native APP and enzyme, or can utilize transgenic animal models expressing the substrate and enzyme. Detection of enzymatic activity can be by analysis of at least one of the cleavage products, for example, by immunoassay, fluorometric or chromogenic assay, HPLC, or other means of detection. Inhibitory compounds are determined as those able to decrease the amount of beta-secretase cleavage product produced in comparison to a control, where beta-secretase mediated cleavage in the reaction system is observed and measured in the absence of inhibitory compounds.

Efficacy reflects a preference for a target tissue. For example, efficacy data values yield information regarding a compound's preference for a target tissue by comparing the compound'd effect on multiple (i.e., two) tissues. See, for example, Dovey et al., J. Neurochemistry, 2001, 76:173-181. Efficacy reflects the ability of compounds to target a specific tissue and create the desired result (e.g., clinically). Efficacious compositions and corresponding methods of treatment are needed to prevent or treat conditions and diseases associated with amyloidosis.

Efficacious compounds of the present invention are those able to decrease the amount of A-beta produced compared to a control, where beta-secretase mediated cleavage is observed and measured in the absence of the compounds. Detection of efficacy can be by analysis of A-beta levels, for example, by immunoassay, fluorometric or chromogenic assay, HPLC, or other means of detection. The efficacy of the compounds of formula (I) was determined as a percentage inhibition corresponding to A-beta concentrations for tissue treated and untreated with a compound of formula (I).

### Beta-Secretase

Various forms of beta-secretase enzyme are known, are available, and useful for assaying enzymatic activity and inhibition of enzyme activity. These include native, recombinant, and synthetic forms of the enzyme. Human beta-secretase is known as Beta Site APP Cleaving Enzyme (BACE), BACE1, Asp2, and memapsin 2, and has been characterized, for example, in U.S. Pat. No. 5,744,346 and published PCT patent applications WO 98/22597, WO 00/03819, WO 01/23533, and WO 00/17369, as well as in literature publications (Hussain et al., 1999, Mol. Cell. Neurosci., 14:419-427; Vassar et al., 1999, Science, 286:735-741; Yan et al., 1999, Nature, 402:533-537; Sinha et al., 1999, Nature, 40:537-540; and Lin et al., 2000, Proceedings Natl. Acad. Sciences USA, 97:1456-1460). Synthetic forms of the enzyme have also been described in, for example, WO 98/22597 and WO 00/17369. Beta-secretase can be extracted and purified from human brain tissue and can be produced in cells, for example mammalian cells expressing recombinant enzyme.

### App Substrate

Assays that demonstrate inhibition of beta-secretase-mediated cleavage of APP can utilize any of the known forms of APP, including the 695 amino acid “normal” isotype described by Kang et al., 1987, Nature, 325:733-6, the 770 amino acid isotype described by Kitaguchi et. al., 1981, Nature, 331:530-532, and variants such as the Swedish Mutation (KM670-1NL) (APP-SW), the London Mutation (V7176F), and others. See, for example, U.S. Pat. No. 5,766,846 and also Hardy, 1992, Nature Genet. 1:233-234, for a review of known variant mutations. Additional useful substrates include the dibasic amino acid modification, APP-KK, disclosed, for example, in WO 00/17369, fragments of APP, and synthetic peptides containing the beta-secretase cleavage site, wild type (WT) or mutated form, (e.g., SW), as described, for example, in U.S. Pat. No. 5,942,400 and WO 00/03819.

The APP substrate contains the beta-secretase cleavage site of APP (KM-DA or NL-DA) for example, a complete APP peptide or variant, an APP fragment, a recombinant or synthetic APP, or a fusion peptide. Preferably, the fusion peptide includes the beta-secretase cleavage site fused to a peptide having a moiety useful for enzymatic assay, for example, having isolation and/or detection properties. A useful moiety can be an antigenic epitope for antibody binding, a label or other detection moiety, a binding substrate, and the like.

### Antibodies

Products characteristic of APP cleavage can be measured by immunoassay using various antibodies, as described, for example, in Pirttila et al., 1999, Neuro. Lett., 249:21-4, and in U.S. Pat. No. 5,612,486. Useful antibodies to detect A-beta include, for example, the monoclonal antibody 6E10 (Senetek, St. Louis, Mo.) that specifically recognizes an epitope on amino acids 1-16 of the A-beta peptide; antibodies 162 and 164 (New York State Institute for Basic Research, Staten Island, N.Y.) that are specific for human A-beta 1-40 and 1-42, respectively; and antibodies that recognize the junction region of A-beta, the site between residues 16 and 17, as described in U.S. Pat. No. 5,593,846. Antibodies raised against a synthetic peptide of residues 591 to 596 of APP and SW192 antibody raised against 590-596 of the Swedish mutation are also useful in immunoassay of APP and its cleavage products, as described in U.S. Pat. Nos. 5,604,102 and 5,721,130.

### Assay Systems

Assays for determining APP cleavage at the beta-secretase cleavage site are well known in the art. Exemplary assays, are described, for example, in U.S. Pat. Nos. 5,744,346 and 5,942,400, and described in the Examples below.

### Cell Free Assays

Exemplary assays that can be used to demonstrate the inhibitory activity of the compounds of the present invention are described, for example, in WO 00/17369, WO 00/03819, and U.S. Pat. Nos. 5,942,400 and 5,744,346. Such assays can be performed in cell-free incubations or in cellular incubations using cells expressing A-beta-secretase and an APP substrate having A-beta-secretase cleavage site.

An APP substrate containing the beta-secretase cleavage site of APP, for example, a complete APP or variant, an APP fragment, or a recombinant or synthetic APP substrate containing the amino acid sequence KM-DA or NL-DA is incubated in the presence of beta-secretase enzyme, a fragment thereof, or a synthetic or recombinant polypeptide variant having beta-secretase activity and effective to cleave the beta-secretase cleavage site of APP, under incubation conditions suitable for the cleavage activity of the enzyme. Suitable substrates optionally include derivatives that can be fusion proteins or peptides that contain the substrate peptide and a modification useful to facilitate the purification or detection of the peptide or its beta-secretase cleavage products. Useful modifications include the insertion of a known antigenic epitope for antibody binding; the linking of a label or detectable moiety, the linking of a binding substrate, and the like.

Suitable incubation conditions for a cell-free in vitro assay include, for example, approximately 200 nM to 10 μM substrate, approximately 10 pM to 200 pM enzyme, and approximately 0.1 nM to 10 μM inhibitor compound, in aqueous solution, at an approximate pH of 4-7, at approximately 37° C., for a time period of approximately 10 min to 3 h. These incubation conditions are exemplary only, and can vary as required for the particular assay components and/or desired measurement system. Optimization of the incubation conditions for the particular assay components should account for the specific beta-secretase enzyme used and its pH optimum, any additional enzymes and/or markers that might be used in the assay, and the like. Such optimization is routine and will not require undue experimentation.

One useful assay utilizes a fusion peptide having maltose binding protein (MBP) fused to the C-terminal 125 amino acids of APP-SW. The MBP portion is captured on an assay substrate by an anti-MBP capture antibody. Incubation of the captured fusion protein in the presence of beta-secretase results in cleavage of the substrate at the beta-secretase cleavage site. Analysis of the cleavage activity can be, for example, by immunoassay of cleavage products. One such immunoassay detects a unique epitope exposed at the carboxy terminus of the cleaved fusion protein, for example, using the antibody SW192. This assay is described, for example, in U.S. Pat. No. 5,942,400.

### Cellular Assay

Numerous cell-based assays can be used to analyze beta-secretase activity and/or processing of APP to release A-beta. Contact of an APP substrate with A-beta-secretase enzyme within the cell and in the presence or absence of a compound inhibitor of the present invention can be used to demonstrate beta-secretase inhibitory activity of the compound. It is preferred that the assay in the presence of a useful inhibitory compound provides at least about 10% inhibition of the enzymatic activity, as compared with a non-inhibited control.

In an embodiment, cells that naturally express beta-secretase are used. Alternatively, cells are modified to express a recombinant beta-secretase or synthetic variant enzyme as discussed above. The APP substrate can be added to the culture medium and is preferably expressed in the cells. Cells that naturally express APP, variant or mutant forms of APP, or cells transformed to express an isoform of APP, mutant or variant APP, recombinant or synthetic APP, APP fragment, or synthetic APP peptide or fusion protein containing the beta-secretase APP cleavage site can be used, provided that the expressed APP is permitted to contact the enzyme and enzymatic cleavage activity can be analyzed.

Human cell lines that normally process A-beta from APP provide useful means to assay inhibitory activities of the compounds employed in the methods of treatment of the present invention. Production and release of A-beta and/or other cleavage products into the culture medium can be measured, for example by immunoassay, such as Western blot or enzyme-linked immunoassay (EIA) such as by ELISA.

Cells expressing an APP substrate and an active beta-secretase can be incubated in the presence of a compound inhibitor to demonstrate inhibition of enzymatic activity as compared with a control. Activity of beta-secretase can be measured by analysis of at least one cleavage product of the APP substrate. For example, inhibition of beta-secretase activity against the substrate APP would be expected to decrease the release of specific beta-secretase induced APP cleavage products such as A-beta.

Although both neural and non-neural cells process and release A-beta, levels of endogenous beta-secretase activity are low and often difficult to detect by EIA. The use of cell types known to have enhanced beta-secretase activity, enhanced processing of APP to A-beta, and/or enhanced production of A-beta are therefore preferred. For example, transfection of cells with the Swedish Mutant form of APP (APP-SW); with APP-KK; or with APP-SW-KK provides cells having enhanced beta-secretase activity and producing amounts of A-beta that can be readily measured.

In such assays, for example, the cells expressing APP and beta-secretase are incubated in a culture medium under conditions suitable for beta-secretase enzymatic activity at its cleavage site on the APP substrate. On exposure of the cells to the compound inhibitor employed in the methods of treatment, the amount of A-beta released into the medium and/or the amount of CTF99 fragments of APP in the cell lysates is reduced as compared with the control. The cleavage products of APP can be analyzed, for example, by immune reactions with specific antibodies, as discussed above.

Preferred cells for analysis of beta-secretase activity include primary human neuronal cells, primary transgenic animal neuronal cells where the transgene is APP, and other cells such as those of a stable 293 cell line expressing APP, for example, APP-SW.

### In Vivo Assays: Animal Models

Various animal models can be used to analyze beta-secretase activity and/or processing of APP to release A-beta, as described above. For example, transgenic animals expressing APP substrate and beta-secretase enzyme can be used to demonstrate inhibitory activity of the compounds of the present invention. Certain transgenic animal models have been described, for example, in U.S. Pat. Nos. 5,877,399, 5,612,486, 5,387,742, 5,720,936, 5,850,003, 5,877,015, and 5,811,633, and in Games et al., 1995, Nature, 373:523. Animals that exhibit characteristics associated with the pathophysiology of Alzheimer's disease are preferred. Administration of the compounds of the present invention to the transgenic mice described herein provides an alternative method for demonstrating the inhibitory activity of the compounds. Administration of the compounds of the present invention in a pharmaceutically effective carrier and via an administrative route that reaches the target tissue in an appropriate therapeutic amount is also preferred.

Inhibition of beta-secretase mediated cleavage of APP at the beta-secretase cleavage site and of A-beta release can be analyzed in these animals by measuring cleavage fragments in the animal's body fluids such as cerebral fluid or tissues. Analysis of brain tissues for A-beta deposits or plaques is preferred.

### A: Enzyme Inhibition Assay

The methods of treatment and compounds of the present invention are analyzed for inhibitory activity by use of the MBP-C125 assay. This assay determines the relative inhibition of beta-secretase cleavage of a model APP substrate, MBP-C125SW, by the compounds assayed as compared with an untreated control. A detailed description of the assay parameters can be found, for example, in U.S. Pat. No. 5,942,400. Briefly, the substrate is a fusion peptide formed of MBP and the carboxy terminal 125 amino acids of APP-SW, the Swedish mutation. The beta-secretase enzyme is derived from human brain tissue as described in Sinha et al., 1999, Nature, 40:537-540 or recombinantly produced as the full-length enzyme (amino acids 1-501), and can be prepared, for example, from 293 cells expressing the recombinant cDNA, as described in WO 00/47618.

Inhibition of the enzyme is analyzed, for example, by immunoassay of the enzyme's cleavage products. One exemplary ELISA uses an anti-MBP capture antibody that is deposited on precoated and blocked 96-well high binding plates, followed by incubation with diluted enzyme reaction supernatant, incubation with a specific reporter antibody, for example, biotinylated anti-SW192 reporter antibody, and further incubation with streptavidin/alkaline phosphatase. In the assay, cleavage of the intact MBP-C125SW fusion protein results in the generation of a truncated amino-terminal fragment, exposing a new SW-192 antibody-positive epitope at the carboxy terminus. Detection is effected by a fluorescent substrate signal on cleavage by the phosphatase. ELISA only detects cleavage following Leu596 at the substrate's APP-SW 751 mutation site.

### Specific Assay Procedure

Compounds of formula (I) are diluted in a 1:1 dilution series to a six-point concentration curve (two wells per concentration) in one row of a 96-well plate per compound tested. Each of the test compounds is prepared in DMSO to make up a 10 mM stock solution. The stock solution is serially diluted in DMSO to obtain a final compound concentration of 200 μM at the high point of a 6-point dilution curve. 10 μL of each dilution is added to each of two wells on row C of a corresponding V-bottom plate to which 190 μL of 52 mM NaOAc, 7.9% DMSO, pH 4.5 are pre-added. The NaOAc diluted compound plate is spun down to pellet precipitant and 20 μL/well is transferred to a corresponding flat-bottom plate to which 30 μL of ice-cold enzyme-substrate mixture (2.5 μL MBP-C125SW substrate, 0.03 μL enzyme and 24.5 μL ice cold 0.09% TX100 per 30 μL) is added. The final reaction mixture of 200 μM compound at the highest curve point is in 5% DMSO, 20 μM NaOAc, 0.06% TX100, at pH 4.5.

Warming the plates to 37° C. starts the enzyme reaction. After 90 min at 37° C., 200 μL/well cold specimen diluent is added to stop the reaction and 20 μL/well was transferred to a corresponding anti-MBP antibody coated ELISA plate for capture, containing 80 μL/well specimen diluent. This reaction is incubated overnight at 4° C. and the ELISA is developed the next day after a 2 h incubation with anti-192SW antibody, followed by Streptavidin-AP conjugate and fluorescent substrate. The signal is read on a fluorescent plate reader.

Relative compound inhibition potency is determined by calculating the concentration of compound that showed a 50% reduction in detected signal (IC₅₀) compared to the enzyme reaction signal in the control wells with no added compound. In this assay, preferred compounds of the present invention exhibit an IC₅₀ of less than 50 μM.

### B: FP BACE Assay: Cell Free Inhibition Assay Utilizing a Synthetic APP Substrate

A synthetic APP substrate that can be cleaved by beta-secretase and having N-terminal biotin and made fluorescent by the covalent attachment of Oregon green at the Cys residue is used to assay beta-secretase activity in the presence or absence of the inhibitory compounds employed in the present invention. Useful substrates include
Biotin-SEVNL-DAEFRC[oregon green]KK,
Biotin-SEVKM-DAEFRC[oregon green]KK,
Biotin-GLNIKTEEISEISY-EVEFRC[oregon green]KK,
Biotin-ADRGLTTRPGSGLTNIKTEEISEVNL-DAEFRC[oregon green]KK, and
Biotin-FVNQHLCoxGSHLVEALY-LVCoxGERGFFYTPKAC[oregon green]KK.

The enzyme (0.1 nM) and test compounds (0.001-100 μM) are incubated in pre-blocked, low affinity, black plates (384 well) at 37° C. for 30 min. The reaction is initiated by addition of 150 μM substrate to a final volume of 30 μL/well. The final assay conditions are 0.001-100 μM compound inhibitor, 0.1 M sodium acetate (pH 4.5), 150 nM substrate, 0.1 nM soluble beta-secretase, 0.001% Tween 20, and 2% DMSO. The assay mixture is incubated for 3 h at 37° C., and the reaction is terminated by the addition of a saturating concentration of immunopure streptavidin. After incubation with streptavidin at room temperature for 15 min, fluorescence polarization is measured, for example, using a LJL Acqurest (Ex485 nm/Em530 nm).

The activity of the beta-secretase enzyme is detected by changes in the fluorescence polarization that occur when the substrate is cleaved by the enzyme. Incubation in the presence or absence of compound inhibitor demonstrates specific inhibition of beta-secretase enzymatic cleavage of its synthetic APP substrate. In this assay, preferred compounds of the present invention exhibit an IC₅₀ of less than 50 μM. More preferred compounds of the present invention exhibit an IC₅₀ of less than 10 μM. Even more preferred compounds of the present invention exhibit an IC₅₀ of less than 5 μM.

### C: Beta-Secretase Inhibition: P26-P4′SW Assay

Synthetic substrates containing the beta-secretase cleavage site of APP are used to assay beta-secretase activity, using the methods described, for example, in published PCT application WO 00/47618. The P26-P4′SW substrate is a peptide of the sequence (biotin)CGGADRGLTTRPGSGLTNIKTEEISEVNLDAEF. The P26-P1 standard has the sequence (biotin)CGGADRGLTTRPGSGLTNIKTEEISEVNL.

Briefly, the biotin-coupled synthetic substrates are incubated at a concentration of from about 0 to about 200 μM in this assay. When testing inhibitory compounds, a substrate concentration of about 1.0 μM is preferred. Test compounds diluted in DMSO are added to the reaction mixture, with a final DMSO concentration of 5%. Controls also contain a final DMSO concentration of 5%. The concentration of beta secretase enzyme in the reaction is varied, to give product concentrations with the linear range of the ELISA assay, about 125 to 2000 pM, after dilution.

The reaction mixture also includes 20 mM sodium acetate, pH 4.5, 0.06% Triton X100, and is incubated at 37° C. for about 1 to 3 h. Samples are then diluted in assay buffer (for example, 145.4 nM sodium chloride, 9.51 mM sodium phosphate, 7.7 mM sodium azide, 0.05% Triton X405, 6 g/L bovine serum albumin, pH 7.4) to quench the reaction, then diluted further for immunoassay of the cleavage products.

Cleavage products can be assayed by ELISA. Diluted samples and standards are incubated in assay plates coated with capture antibody, for example, SW192, for about 24 h at 4° C. After washing in TTBS buffer (150 mM sodium chloride, 25 mM Tris, 0.05% Tween 20, pH 7.5), the samples are incubated with streptavidin-AP according to the manufacturer's instructions. After a 1 h incubation at room temperature, the samples are washed in TTBS and incubated with fluorescent substrate solution A (31.2 g/L 2-amino-2-methyl-1-propanol, 30 mg/L, pH 9.5). Reaction with streptavidin-alkaline phosphate permits detection by fluorescence. Compounds that are effective inhibitors of beta-secretase activity demonstrate reduced cleavage of the substrate as compared to a control.

### D: Assays using Synthetic Oligopeptide-Substrates

Synthetic oligopeptides are prepared incorporating the known cleavage site of beta-secretase, and optionally include detectable tags, such as fluorescent or chromogenic moieties. Examples of such peptides, as well as their production and detection methods, are described in U.S. Pat. No. 5,942,400. Cleavage products can be detected using high performance liquid chromatography, or fluorescent or chromogenic detection methods appropriate to the peptide to be detected, according to methods well known in the art.

By way of example, one such peptide has the sequence SEVNL-DAEF, and the cleavage site is between residues 5 and 6. Another preferred substrate has the sequence ADRGLTTRPGSGLTNIKTEEISEVNL-DAEF, and the cleavage site is between residues 26 and 27.

These synthetic APP substrates are incubated in the presence of beta-secretase under conditions sufficient to result in beta-secretase mediated cleavage of the substrate. Comparison of the cleavage results in the presence of a compound inhibitor to control results provides a measure of the compound's inhibitory activity.

### E: Inhibition of Beta-Secretase Activity-Cellular Assay

An exemplary assay for the analysis of inhibition of beta-secretase activity utilizes the human embryonic kidney cell line HEKp293 (ATCC Accession No. CRL-1573) transfected with APP751 containing the naturally occurring double mutation Lys651Met652 to Asn651Leu652 (numbered for APP751), commonly called the Swedish mutation and shown to overproduce A-beta (Citron et al., 1992, Nature, 360:672-674), as described in U.S. Pat. No. 5,604,102.

The cells are incubated in the presence/absence of the inhibitory compound (diluted in DMSO) at the desired concentration, generally up to 10 μg/mL. At the end of the treatment period, conditioned media is analyzed for beta-secretase activity, for example, by analysis of cleavage fragments. A-beta can be analyzed by immunoassay, using specific detection antibodies. The enzymatic activity is measured in the presence and absence of the compound of formula (I) to demonstrate specific inhibition of beta-secretase mediated cleavage of APP substrate.

### F: Inhibition of Beta-Secretase in Animal Models of Alzheimer's Disease

Various animal models can be used to screen for inhibition of beta-secretase activity. Examples of animal models useful in the present invention include mouse, guinea pig, dog, and the like. The animals used can be wild type, transgenic, or knockout models. In addition, mammalian models can express mutations in APP, such as APP695-SW and the like as described herein. Examples of transgenic non-human mammalian models are described in U.S. Pat. Nos. 5,604,102, 5,912,410 and 5,811,633.

PDAPP mice, prepared as described in Games et al., 1995, Nature, 373:523-527 are useful to analyze in vivo suppression of A-beta release in the presence of putative inhibitory compounds. As described in U.S. Pat. No. 6,191,166, 4-month-old. PDAPP mice are administered a compound of formula (I) formulated in a vehicle, such as corn oil. The mice are dosed with the compound (1-30 mg/mL, preferably 1-10 mg/mL). After a designated time, e.g., 3-10 h, the brains are analyzed.

Transgenic animals are administered an amount of a compound formulated in a carrier suitable for the chosen mode of administration. Control animals are untreated, treated with vehicle, or treated with an inactive compound. Administration can be acute, (i.e. single dose or multiple doses in one day), or can be chronic, (i.e. dosing is repeated daily for a period of days). Beginning at time 0, brain tissue or cerebral fluid is obtained from selected animals and analyzed for the presence of APP cleavage peptides, including A-beta, for example, by immunoassay using specific antibodies for A-beta detection. At the end of the test period, animals are sacrificed and brain tissue or cerebral fluid is analyzed for the presence of A-beta and/or beta-amyloid plaques. The tissue is also analyzed for necrosis.

Reduction of A-beta in brain tissues or cerebral fluids and reduction of beta-amyloid plaques in brain tissue are assessed by administering the compounds of formula (I), or pharmaceutical compositions comprising compounds of formula (I) to animals and comparing the data with that from non-treated controls.

### G: Inhibition of A-beta Production in Human Patients

Patients suffering from Alzheimer's disease demonstrate an increased amount of A-beta in the brain. Alzheimer's disease patients are subjected to a method of treatment of the present invention, (i.e. administration of an amount of the compound inhibitor formulated in a carrier suitable for the chosen mode of administration). Administration is repeated daily for the duration of the test period. Beginning on day 0, cognitive and memory tests are performed, for example, once per month.

Patients administered the compounds of formula (I) are expected to demonstrate slowing or stabilization of disease progression as analyzed by a change in at least one of the following disease parameters: A-beta present in cerebrospinal fluid or plasma; brain or hippocampal volume; A-beta deposits in the brain; amyloid plaque in the brain; or scores for cognitive and memory function, as compared with control, non-treated patients.

### H: Prevention of A-beta Production in Patients at Risk for Alzheimer's Disease

Patients predisposed or at risk for developing Alzheimer's disease can be identified either by recognition of a familial inheritance pattern, for example, presence of the Swedish Mutation, and/or by monitoring diagnostic parameters. Patients identified as predisposed or at risk for developing Alzheimer's disease are administered an amount of the compound inhibitor formulated in a carrier suitable for the chosen mode of administration. Administration is repeated daily for the duration of the test period. Beginning on day 0, cognitive and memory tests are performed, for example, once per month.

Patients subjected to a method of treatment of the present invention (i.e., administration of a compound of formula (I)) are expected to demonstrate slowing or stabilization of disease progression as analyzed by a change in at least one of the following disease parameters: A-beta present in cerebrospinal fluid or plasma; brain or hippocampal volume; amyloid plaque in the brain; or scores for cognitive and memory function, as compared with control, non-treated patients.

### I: Efficacy of Compounds to Inhibit A-beta Concentration

The invention encompasses compounds of formula (I) that are efficacious. Efficacy is calculated as a percentage of concentrations as follows:
Efficacy=(1−(total A-beta in dose group/total A-beta in vehicle control))*100%
wherein the “total A-beta in dose group” equals the concentration of A-beta in the tissue, (e.g., rat brain) treated with the compound, and the “total A-beta in vehicle control” equals the concentration of A-beta in the tissue, yielding a % inhibition of A-beta production. Statistical significance is determined by p-value<0.05 using the Mann Whitney t-test. See, for example, Dovey et al., J. Neurochemistry, 2001, 76:173-181.

Where indicated, diastereomers were separated by reverse phase HPLC using the noted methods. The first isomer collected in each case was designated Diastereomer A, and the second isomer Diastereomer B. Where indicated, specific formula (I) compound examples represent single diastereomers (e.g., diastereomer A).

Efficacy For Exemplary Formula (I) Compounds

| | | Efficacy | |
|---|---|---|---|
| | | (% Inhibition, | |
| | | 100 mg/kg) | |
| Example No. | Compound | cortex | plasma |
| 91.1 | 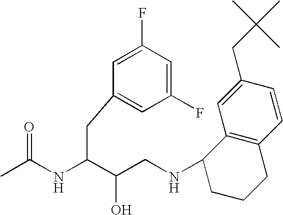 | 47 | 63 |
| | N-{1-(3,5-Difluoro-benzyl)-3-[7-(2,2- | | |
| | dimethyl-propyl)-1,2,3,4-tetrahydro- | | |
| | naphthalen-1-ylamino]-2-hydroxy-propyl}- | | |
| | acetamide | | |
| 91.2 Diastereomer A | 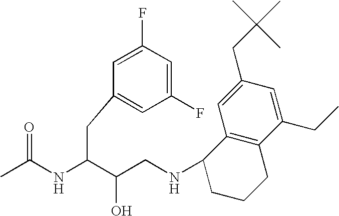 | 44 | 57 |
| | N-{1-(3,5-Difluoro-benzyl)-3-[7-(2,2- | | |
| | dimethyl-propyl)-5-ethyl-1,2,3,4-tetrahydro- | | |
| | naphthalen-1-ylamino]-2-hydroxy-propyl}- | | |
| | acetamide | | |
| 91.3 | 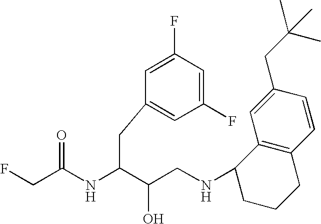 | 14 | 23 |
| | N-{1-(3,5-Difluoro-benzyl)-3-[7-(2,2- | | |
| | dimethyl-propyl)-1,2,3,4-tetrahydro- | | |
| | naphthalen-1-ylamino]-2-hydroxy-propyl}-2- | | |
| | fluoro-acetamide | | |
| 91.4 Diastereomer B | 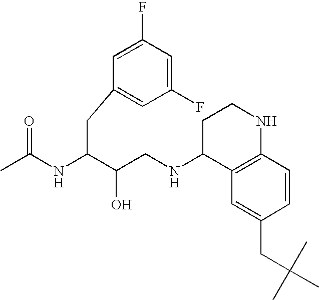 | 18 | 59 |
| | N-{1-(3,5-Difluoro-benzyl)-3-[6-(2,2- | | |
| | dimethyl-propyl)-1,2,3,4-tetrahydro-quinolin- | | |
| | 4-ylamino]-2-hydroxy-propyl}-acetamide | | |
| 91.5 Diastereomer A | 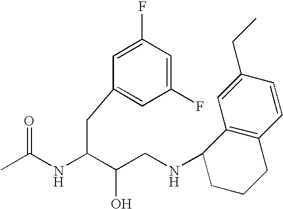 | 35 | 44 |
| | N-[1-(3,5-Difluoro-benzyl)-3-(7-ethyl-1,2,3,4- | | |
| | tetrahydro-naphthalen-1-ylamino)-2- | | |
| | hydroxy-propyl]-acetamide | | |

### J: Selectivity of Compounds for Inhibiting BACE over Aspartyl Proteases

The compounds of formula (I) can be selective for beta-secretase versus catD. Wherein the ratio of catD:beta-secretase is greater than 1, selectivity for BACE versus catD is calculated as follows:
Selectivity=(IC₅₀ for catD/IC₅₀ for beta-secretase)*100%
wherein IC₅₀ is the concentration of compound necessary to decrease the level of catD or beta-secretase by 50%. Selectivity is reported as the ratio of IC₅₀(catD):IC₅₀(BACE).

The compounds of formula (I) can be selective for beta-secretase versus catE. Wherein the ratio of catE:beta-secretase is greater than 1, selectivity is calculated as follows:
Selectivity=(IC₅₀ for catE/IC₅₀ for beta-secretase)*100%
wherein IC₅₀ is the concentration of compound necessary to decrease the level of catE or beta-secretase by 50%. Selectivity is reported as the ratio of IC₅₀(catE):IC₅₀(BACE).

Pharmacokinetic parameters were calculated by a non-compartmental approach. See, for example, Gibaldi, M. and Perrier, D., Pharmacokinetics, Second Edition, 1982, Marcel Dekker Inc., New York, N.Y., pp 409-418.

In the following examples, each value is an average of four experimental runs and multiple values for one compound indicate that more than one experiment was conducted.

Selectivity For Exemplary Formula (I) Compounds

| | | Selectivity |
|---|---|---|
| Example | | IC₅₀ catD/ |
| No. | Compound | IC₅₀ BACE |
| 91.6 | 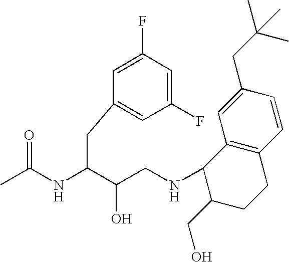 | 4.4 |
| | N-{1-(3,5-Difluoro-benzyl)-3-[7-(2,2-dimethyl- | |
| | propyl)-2-hydroxymethyl-1,2,3,4-tetrahydro- | |
| | naphthalen-1-ylamino]-2-hydroxy-propyl}- | |
| | acetamide | |
| 91.7 | 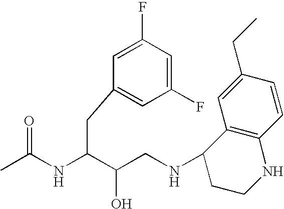 | 2.1 |
| | N-[1-(3,5-Difluoro-benzyl)-3-(6-ethyl-1,2,3,4- | |
| | tetrahydro-quinolin-4-ylamino)-2-hydroxy- | |
| | propyl]-acetamide | |

### K: Oral Bioavailability of Compounds for Inhibitinq Amyloidosis

The invention encompasses compounds of formula (I) that are orally bioavailable. Generally, oral bioavailability is defined as the fraction of orally administered dose reaching systemic circulation. Oral bioavailability can be determined following both an intravenous (IV) and oral (PO) administration of a test compound.

Oral bioavailability was determined in the male Sprague-Dawley rat following both IV and PO administration of test compound. Two month-old male rats (250-300 g) were surgically implanted with polyethylene (PE-50) cannula in the jugular vein while under isoflurane anesthesia the day before the in-life phase. Animals were fasted overnight with water ad libitum, then dosed the next day. The dosing regime. consisted of either a 5 mg/kg (2.5 mL/kg) IV dose (N=3) administered to the jugular vein cannula, then flushed with saline, or a 10 mg/kg (5 mL/kg) PO dose (N=3) by esophageal gavage. Compounds were formulated with 10% Solutol in 5% dextrose at 2 mg/mL. Subsequent to dosing, blood was collected at 0.016 (IV only), 0.083, 0.25, 0.5, 1, 3, 6, 9, and 24 h post administration, and heparinized plasma was recovered following centrifugation.

Compounds were extracted from samples following precipitation of the plasma proteins by methanol. The resulting supernatants were evaporated to dryness and reconstituted with chromatographic mobile phase (35% acetonitrile in 0.1% formic acid) and injected onto a reverse phase C₁₈ column (2×50 mm, 5 μm, BDS Hypersil). Detection was facilitated with a multi-reaction-monitoring experiment on a tandem triple quadrupole mass spectrometer (LC/MS/MS) following electrospray ionization. Experimental samples were compared to calibration curves prepared in parallel with aged match rat plasma and quantitated with a weighted 1/x linear regression. The lower limit of quantization (LOQ) for the assay was typically 0.5 ng/mL.

Oral bioavailability (% F) is calculated from the dose normalized ratio of plasma exposure following oral administration to the intravenous plasma exposure in the rat by the following equation
% F=(AUCₚₒ/AUCᵢᵥ)×(Dᵢᵥ/Dₚₒ)×100%
where D is the dose and AUC is the area-under-the-plasma-concentration-time-curve from 0 to 24 h. AUC is calculated from the linear trapezoidal rule by AUC=((C₂+C₁)/2)×(T₂−T₁) where C is concentration and T is time.

Pharmacokinetic parameters were calculated by a non-compartmental approach. See, for example, Gibaldi, M. and Perrier, D., Pharmacokinetics, Second Edition, 1982, Marcel Dekker Inc., New York, N.Y., pp 409-418.

Oral Bioavailability For Exemplary Formula (I) Compounds

| | | BACE | Cell | |
|---|---|---|---|---|
| | | EC₅₀ | EC₅₀ | |
| Example No. | Compound | (nM) | (nM) | % F |
| 91.8 Diastereomer A | 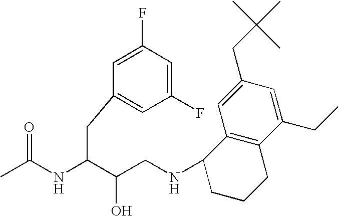 | 12 | 32 | 23.3 |
| | N-{1-(3,5-Difluoro-benzyl)-3-[7- | | | |
| | (2,2-dimethyl-propyl)-5-ethyl- | | | |
| | 1,2,3,4-tetrahydro-naphthalen-1- | | | |
| | ylamino]-2-hydroxy-propyl}- | | | |
| | acetamide | | | |
| 91.9 | 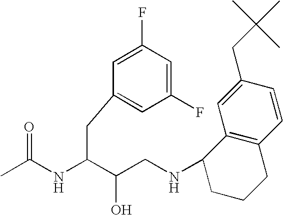 | 9 | 9 | 15 |
| | N-{1-(3,5-Difluoro-benzyl)-3-[7- | | | |
| | (2,2-dimethyl-propyl)-1,2,3,4- | | | |
| | tetrahydro-naphthalen-1-ylamino]- | | | |
| | 2-hydroxy-propyl}-acetamide | | | |
| 91.10 Diastereomer B | 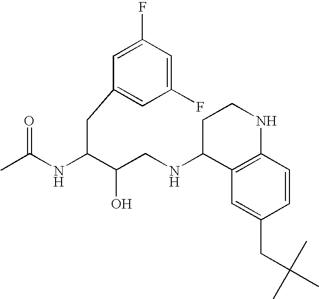 | 190 | 13 | 5 |
| | N-{1-(3,5-Difluoro-benzyl)-3-[6- | | | |
| | (2,2-dimethyl-propyl)-1,2,3,4- | | | |
| | tetrahydro-quinolin-4-ylamino]-2- | | | |
| | hydroxy-propyl}-acetamide | | | |
| 91.11 | 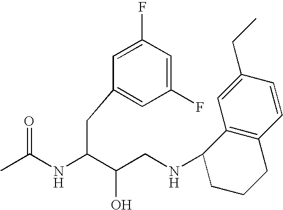 | 210 | 48 | 14.3 |
| | N-[1-(3,5-Difluoro-benzyl)-3-(7- | | | |
| | ethyl-1,2,3,4-tetrahydro- | | | |
| | naphthalen-1-ylamino)-2-hydroxy- | | | |
| | propyl]-acetamide | | | |

### L: Brain Uptake

The invention encompasses beta-secretase inhibitors that can readily cross the blood-brain barrier. Factors that affect a compound's ability to cross the blood-brain barrier include a compound's molecular weight, Total Polar Surface Area (TPSA), and log P (lipophilicity). See, e.g., Lipinski, C. A., et al., Adv. Drug Deliv. Reviews, 23:3-25 (1997). One of ordinary skill in the art will be aware of methods for determining characteristics allowing a compound to cross the blood-brain barrier. See, for example, Murcko et al., Designing Libraries with CNS Activity, J. Med. Chem., 42 (24), pp. 4942-51 (1999). Calculations of log P values were performed using the Daylight c log P program (Daylight Chemical Information Systems, Inc.). See, for example, Hansch, C., et al., Substituent Constants for Correlation Analysis in Chemistry and Biology, Wiley, New York (1979); Rekker, R., The Hydrophobic Fragmental Constant, Elsevier, Amsterdam (1977); Fujita, T., et al., J. Am. Chem. Soc., 86, 5157 (1964). TPSA was calculated according to the methodology outlined in Ertl, P., et al., J. Med. Chem., 43:3714-17 (2000).

The following assay was employed to determine the brain penetration of compounds encompassed by the present invention.

In-life phase: Test compounds were administered to CF-1 (20-30 g) mice at 10 μmol/kg (4 to 7 mg/kg) following IV administration in the tail vein. Two time-points, 5 and 60 min, were collected post dose. Four mice were harvested for heparinized plasma and non-perfused brains at each time-point for a total of 8 mice per compound.

Analytical phase: Samples were extracted and evaporated to dryness, then reconstituted and injected onto a reverse phase chromatographic column while monitoring the effluent with a triple quadrupole mass spectrometer. Quantitation was then performed with a 1/x² weighted fit of the least-squares regression from calibration standards prepared in parallel with the in vivo samples. The LOQ is generally 1 ng/mL and 0.5 ng/g for the plasma and brain respectively. Data was reported in micromolar (μM) units. Brain levels were corrected for plasma volumes (16 μL/g).

Results: Comparison of a compound's brain concentration level to two marker compounds, Indinavir and Diazepam, demonstrates the ability in which the compounds of the present invention can cross the blood-brain barrier. Indinavir (HIV protease inhibitor) is a poor brain penetrant marker and Diazepam is a blood flow limited marker. The concentration levels of Indinavir in the brain at 5 and 60 min were 0.165 μM and 0.011 μM, respectively. The concentration levels of Diazepam at 5 and 60 min were 5.481 μM and 0.176 μM, respectively.

Brain Uptake For Exemplary Formula (I) Compounds

| Example | | [Brain/[Plasma] | | | |
|---|---|---|---|---|---|
| No. | Compound | 5 min | 60 min | clogP | TPSA |
| 91.12 | 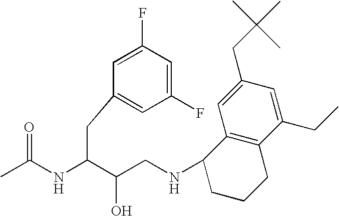 | 2.2 | 5.4 | 6.4 | 61.4 |
| | N-{1-(3,5-Difluoro-benzyl)-3- | | | | |
| | [7-(2,2-dimethyl-propyl)-5- | | | | |
| | ethyl-1,2,3,4-tetrahydro- | | | | |
| | naphthalen-1-ylamino]-2- | | | | |
| | hydroxy-propyl}-acetamide | | | | |
| 91.13 | 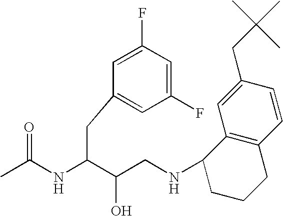 | 1.0 | 3.5 | 5.6 | 61.4 |
| | N-{1-(3,5-Difluoro-benzyl)-3- | | | | |
| | [7-(2,2-dimethyl-propyl)- | | | | |
| | 1,2,3,4-tetrahydro- | | | | |
| | naphthalen-1-ylamino]-2- | | | | |
| | hydroxy-propyl}-acetamide | | | | |
| 91.14 | 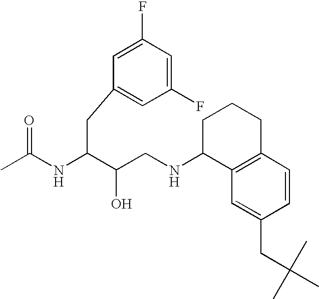 | 0.2 | 2.2 | 4.0 | 73.4 |
| | N-{1-(3,5-Difluoro-benzyl)-3- | | | | |
| | [6-(2,2-dimethyl-propyl)- | | | | |
| | 1,2,3,4-tetrahydro-quinolin-4- | | | | |
| | ylamino]-2-hydroxy-propyl}- | | | | |
| | acetamide | | | | |
| 91.15 | 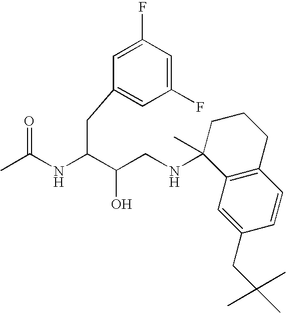 | 1.1 | 2.0 | 6.1 | 61.4 |
| | N-{1-(3,5-Difluoro-benzyl)-3- | | | | |
| | [7-(2,2-dimethyl-propyl)-1- | | | | |
| | methyl-1,2,3,4-tetrahydro- | | | | |
| | naphthalen-1-ylamino]-2- | | | | |
| | hydroxy-propyl}-acetamide | | | | |
| 91.16 | 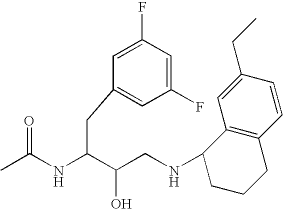 | 0.9 | 2.2 | 4.2 | 61 |
| | N-[1-(3,5-Difluoro-benzyl)-3- | | | | |
| | (7-ethyl-1,2,3,4-tetrahydro- | | | | |
| | naphthalen-1-ylamino)-2- | | | | |
| | hydroxy-propyl]-acetamide | | | | |

The present invention has been described with reference to various specific and preferred embodiments and techniques. However, it should be understood that many variations and modifications may be made while remaining within the spirit and scope of the present invention.

Unless defined otherwise, all scientific and technical terms used herein have the same meaning as commonly understood by one of skill in the art to which this invention belongs. Although methods and materials similar or equivalent to those described herein can be used in the practice or testing of the present invention, suitable methods and materials are described above. Additionally, the materials, methods, and examples are illustrative only and not intended to be limiting. All publications, patent applications, patents, and other references mentioned herein are incorporated by reference in their entirety. In case of conflict, the present specification, including definitions, will control.

1. A method of preventing or treating at least one condition which benefits from inhibition of at least one aspartyl-protease, comprising:
administering to a host a composition comprising a therapeutically effective amount of at least one compound of formula (I), 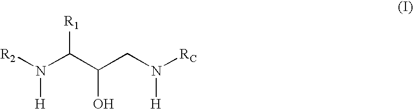
or a pharmaceutically acceptable salt thereof; wherein
R₁ is selected from 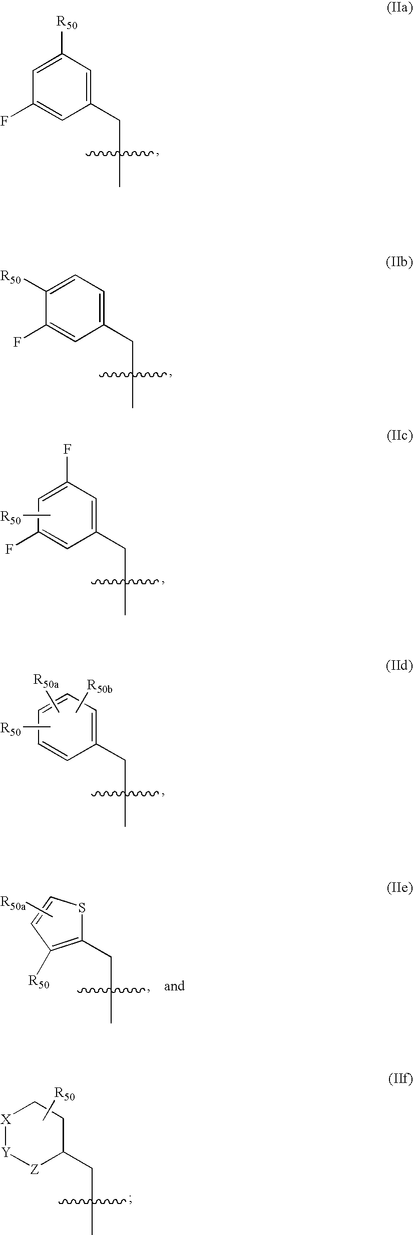
wherein
X, Y, and Z are independently selected from
—C(H)₀₋₂—,
—O—,
—C(O)—,
—NH—, and
—N—;
wherein at least one bond of the (IIf) ring may optionally be a double bond;
R₅₀, R₅₀ₐ, and R_{50b} are independently selected from
—H,
-halogen,
—OH,
—SH,
—CN,
—C(O)-alkyl,
—NR₇R₈,
—S(O)₀₋₂-alkyl,
-alkyl,
-alkoxy,
—O-benzyl optionally substituted with at least one substituent independently selected from —H, —OH, and alkyl,
—C(O)—NR₇R₈,
-alkyloxy,
-alkoxyalkoxyalkoxy, and
-cycloalkyl;
wherein the alkyl, alkoxy, and cycloalkyl groups within R₅₀, R₅₀ₐ, and R_{50b} are optionally substituted with at least one substituent independently selected from alkyl, halogen, —OH, —NR₅R₆, —NR₇R₈, —CN, haloalkoxy, and alkoxy;
R₅ and R₆ are independently selected from —H and alkyl; or
R₅ and R₆, and the nitrogen to which they are attached, form a 5 or 6 membered heterocycloalkyl ring;
R₇ and R₈ are independently selected from
—H,
-alkyl optionally substituted with at least one group independently selected from —OH, —NH₂, and halogen,
-cycloalkyl, and
-alkyl-O-alkyl;
R₂ is selected from —C(O)—CH₃, —C(O)—CH₂(halogen), —C(O)—CH(halogen)₂, 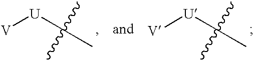
wherein
U is selected from —C(O)—, —C(═S)—, —S(O)₀₋₂—, —C═N—R₂₁—, —C═N—OR₂₁—, —C(O)—NR₂₀—, —C(O)—O—, —S(O)₂—NR₂₀—, and —S(O)₂—O—;
U′ is selected from —C(O)—, —C═N—R₂₁—, —C═N—OR₂₁—, —C(O)—NR₂₀—, and —C(O)—O—;
V is selected from aryl, heteroaryl, cycloalkyl, heterocycloalkyl, —[C(R₄)(R_{4′})]₁₋₃-D, and -(T)₀₋₁-R_{N};
V′ is selected from -(T)₀₋₁-R_{N′};
wherein the aryl, heteroaryl, cycloalkyl, and heterocycloalkyl groups included within V and V′ are optionally substituted with 1 or 2 R_{B} groups;
wherein at least one carbon of the aryl, heteroaryl, cycloalkyl, and heterocycloalkyl groups included within V and V′ are optionally replaced with —N—, —O—, —NH—, —C(O)—, —C(S)—, —C(═N—H)—, —C(═N—OH)—, —C(═N-alkyl)-, or —C(═N—O-alkyl)-;
R_{B} at each occurrence is independently selected from halogen, —OH, —CF₃, —OCF₃, —O-aryl, —CN, —NR₁₀₁R′₁₀₁, -alkyl, -alkoxy, —(CH₂)₀₋₄—(C(O))₀₋₁—(O)₀₋₁-alkyl, —C(O)—OH, —(CH₂)₀₋₃-cycloalkyl, -aryl,
-heteroaryl, and -heterocycloalkyl;
wherein, the alkyl, alkoxy, cycloalkyl, aryl, heteroaryl, or heterocycloalkyl groups included within R_{B} are optionally substituted with 1 or 2 groups independently selected from —C₁-C₄ alkyl, —C₁-C₄ alkoxy, —C₁-C₄ haloalkyl, —C₁-C₄ haloalkoxy, -halogen, —OH, —CN, and
—NR₁₀₁R′₁₀₁;
R₁₀₁ and R′₁₀₁ are independently selected from —H, -alkyl, —(C(O))₀₋₁—(O)₀₋₁-alkyl, —C(O)—OH, and -aryl;
R₄ and R₄ are independently selected from -hydrogen, -alkyl, —(CH₂)₀₋₃-cycloalkyl, —(CH₂)₀₋₃—OH, -fluorine, —CF₃, —OCF₃, —O-aryl, -alkoxy, —C₃-C₇ cycloalkoxy, -aryl, and -heteroaryl, or
R₄ and R₄ are taken together with the carbon to which they are attached to form a 3, 4, 5, 6, or 7 membered carbocyclic ring wherein 1, 2, or 3 carbons of the ring is optionally replaced with —O—, —N(H)—, —N(alkyl)-, —N(aryl)-, —C(O)—, or —S(O)₀₋₂;
D is selected from aryl, heteroaryl, cycloalkyl, and heterocycloalkyl, wherein the aryl, heteroaryl, cycloalkyl, and heterocycloalkyl are optionally substituted with 1 or 2 R_{B} groups;
T is selected from —NR₂₀— and —O—;
R₂₀ is selected from H, —CN, -alkyl, -haloalkyl, and -cycloalkyl;
R₂₁ is selected from —H, -alkyl, -haloalkyl, and -cycloalkyl;
R_{N} is selected from —OH, —NH₂, —NH(alkyl), —NH(cycloalkyl), —N(alkyl)(alkyl), —N (alkyl)(cycloalkyl), —N (cycloalkyl)(cycloalkyl), —R′₁₀₀, alkyl-R₁₀₀, —(CRR′)₁₋₆R′₁₀₀, —(CRR′)₀₋₆R₁₀₀, —(CRR′)₁₋₆—O—R′₁₀₀, —(CRR′)₁₋₆—S—R′₁₀₀, —(CRR′)₁₋₆—C(O)—R₁₀₀, —(CRR′)₁₋₆—SO₂—R₁₀₀, and —(CRR′)₁₋₆—NR₁₀₀—R′₁₀₀, —(CRR′)₁₋₆—P(O)(O-alkyl)₂, alkyl-O-alkyl-C(O)OH, and —CH(R_{E1})—(CH₂)₀₋₃-E₁-E₂-E₃;
R_{N′}is —SO₂R′₁₀₀;
R and R′ are independently selected from -hydrogen, —C₁-C₁₀ alkyl (optionally substituted with at least one group independently selected from —OH, —C₁-C₁₀ alkylaryl, and —C₁-C₁₀ alkylheteroaryl);
R₁₀₀ and R′₁₀₀ are independently selected from
-cycloalkyl,
-heterocycloalkyl,
-aryl,
-heteroaryl,
-alkoxy,
-aryl-W-aryl,
-aryl-W-heteroaryl,
-aryl-W-heterocycloalkyl,
-heteroaryl-W-aryl,
-heteroaryl-W-heteroaryl,
-heteroaryl-W-heterocycloalkyl,
-heterocycloalkyl-W-aryl,
-heterocycloalkyl-W-heteroaryl,
-heterocycloalkyl-W-heterocycloalkyl,
—W—R₁₀₂,
—CH[(CH₂)₀₋₂—O—R₁₅₀]—(CH₂)₀₋₂-aryl,
—CH[(CH₂)₀₋₂—O—R₁₅₀]—(CH₂)₀₋₂-cycloalkyl,
—CH[(CH₂)₀₋₂—O—R₁₅₀]—(CH₂)₀₋₂-heterocycloalkyl,
—CH[(CH₂)₀₋₂—O—R₁₅₀]—(CH₂)₀₋₂-heteroaryl,
—C₁-C₁₀ alkyl optionally substituted with 1, 2, or 3 R₁₁₅ groups, wherein 1, 2, or 3 carbons of the alkyl group are optionally replaced with a group independently selected from —C(O)— and —NH—,
-alkyl-O-alkyl optionally substituted with 1, 2, or 3 R,₁₁₅ groups,
-alkyl-S-alkyl optionally substituted with 1, 2, or 3 R₁₁₅ groups, and
-cycloalkyl optionally substituted with 1, 2, or 3 R₁₁₅ groups;
wherein the ring portions of each group included within R₁₀₀ and R′₁₀₀ are optionally substituted with 1, 2, or 3 groups independently selected- from —OR, —NO₂, -halogen, —CN, —OCF₃, —CF₃, —(CH₂)₀₋₄—O—P(═O)(OR)(OR′), —(CH₂)₀₋₄—C(O)—NR₁₀₅R′₁₀₅, —(CH₂)₀₋₄—O—(CH₂)₀₋₄—C(O)NR₁₀₂R₁₀₂′, —(CH₂)₀₋₄—C(O)—(C₁-C₁₂ alkyl), —(CH₂)₀₋₄—C(O)—(CH₂)₀₋₄-cycloalkyl, —(CH₂)₀₋₄—R₁₁₀, —(CH₂)₀₋₄—R₁₂₀, —(CH₂)₀₋₄—R₁₃₀, —(CH₂)₀₋₄—C(O)—R₁₁₀, —(CH₂)₀₋₄—C(O)—R₁₂₀, —(CH₂)₀₋₄—C(O)—R₁₃₀, —(CH₂)₀₋₄—C(O)—R₁₄₀, —(CH₂)₀₋₄—C(O)—O—R₁₅₀, —(CH₂)₀₋₄—SO₂—NR₁₀₅R′₁₀₅, —(CH₂)₀₋₄—SO—(C₁-C₈ alkyl), —(CH₂)₀₋₄—SO₂—(C₁-C₁₂ alkyl), —(CH₂)₀₋₄—SO₂—(CH₂)₀₋₄-cycloalkyl, —(CH₂)-₀₋₄—N(R₁₅₀)—C(O)—O—R₁₅₀, —(CH₂)₀₋₄—N(R₁₅₀)—C(O)—N(R₁₅₀)₂, —(CH₂)₀₋₄—N(R₁₅₀)—CS—N(R₁₅₀)₂, —(CH₂)₀₋₄—N(R₁₅₀)—C(O)—R₁₀₅, —(CH₂)₀₋₄—NR₁₀₅R′₁₀₅, —(CH₂)₀₋₄—R₁₄₀, —(CH₂)₀₋₄—O—C(O)-(alkyl), —(CH₂)₀₋₄—O—P(O)—(O—R₁₁₀)2, —(CH₂)₀₋₄—O—C(O)—N(R₁₅₀)₂, —(CH₂)₀₋₄—O—CS—N(R₁₅₀)₂, —(CH₂)₀₋₄—O—(R₁₅₀), —(CH₂)₀₋₄—O—R₁₅₀′—C(O)OH, —(CH₂)₀₋₄—S—(R₁₅₀), —(CH₂)₄₋₀—N(R₁₅₀)—SO₂—R₁₀₅, —(CH₂)₀₋₄-cycloalkyl, and —(C₁-C₁₀)-alkyl;
R_{E1} is selected from —H, —OH, —NH₂, —NH—(CH₂)₀₋₃—R_{E2}, —NHR_{E8}, —NR_{E350}C(O)R_{E5}, —C₁-C₄-alkyl-NHC(O)R_{E5}, —(CH₂)₀₋₄R_{E8}, —O—(C₁-C₄ alkanoyl), —C₆-C₁₀ (aryloxy optionally substituted with 1, 2, or 3 groups that are independently selected from halogen, —C₁-C₄ alkyl, —CO₂H, —C(O)—C₁-C₄ alkoxy, and —C₁-C₄ alkoxy), alkoxy, -aryl-(C₁-C₄ alkoxy), —NR_{E350}CO₂R_{E351}, —C₁-C₄ alkyl-NR_{E350}CO₂R_{E351}, —CN, —CF₃, —CF₂-CF₃, —C≡CH, —CH₂—CH═CH₂, —(CH₂)₁₋₄—R_{E2}, —(CH₂)₁₋₄—NH—R_{E2}, —O—(CH₂)₀₋₃—R_{E2}, —S—(CH₂)₀₋₃—R_{E2}, —(CH₂)₀₋₄—NHC(O)—(CH₂)₀₋₆—R_{E352}, and —(CH₂)₀₋₄—(R_{E353})₀₋₁—(CH₂)₀₋₄—R_{E354};
R_{E2} is selected from —SO₂—(C₁-C₈ alkyl), —SO—(C₁-C₈ alkyl), —S—(C₁-C₈ alkyl), —S—C(O)-alkyl, —SO₂—NR_{E3}R_{E4}, —C(O)—C₁-C₂ alkyl, and —C(O)—NR_{E4}R_{E10};
R_{E3} and R_{E4} are independently selected from —H, —C₁-C₃ alkyl, and —C₃-C₆ cycloalkyl;
R_{E10} is selected from alkyl, arylalkyl, alkanoyl, and arylalkanoyl;
R_{E5} is selected from cycloalkyl, alkyl (optionally substituted with 1, 2, or 3 groups that are independently selected from halogen, —NR_{E6}R_{E7}, C₁-C₄ alkoxy, —C₅-C₆ heterocycloalkyl, —C₅-C₆ heteroaryl, —C₆-C₁₀ aryl, —C₃-C₇ cycloalkyl C₁-C₄ alkyl, —S—C₁-C₄ alkyl, —SO₂—C₁-C₄ alkyl, —CO₂H, —C(O)NR_{E6}R_{E7}, —CO₂—C₁-C₄ alkyl, and —C₆-C₁₀ aryloxy), heteroaryl (optionally substituted with 1, 2, or 3 groups that are independently selected from —C₁-C₄ alkyl, —C₁-C₄ alkoxy, halogen, —C₁-C₄ haloalkyl, and —OH), heterocycloalkyl (optionally substituted with 1, 2, or 3 groups independently selected from —C₁-C₄ alkyl, —C₁-C₄ alkoxy, halogen, and —C₂-C₄ alkanoyl), aryl (optionally substituted with 1, 2, 3, or 4 groups independently selected from halogen, —OH, —C₁-C₄ alkyl, —C₁-C₄ alkoxy, and —C₁-C₄ haloalkyl), and —NR_{E6}R_{E7};
R_{E6} and R_{E7} are independently selected from —H, alkyl, alkanoyl, aryl, —SO₂—C₁-C₄ alkyl, and -aryl-C₁-C₄ alkyl;
R_{E8} is selected from —SO₂-heteroaryl, —SO₂-aryl, —SO₂-heterocycloalkyl, —SO₂-C₁-C₁₀ alkyl, —C(O)NHR_{E9}, heterocycloalkyl, —S— alkyl, and —S—C₂-C₄ alkanoyl;
R_{E9} is selected from H, alkyl, and -aryl C₁-C₄ alkyl;
R_{E350} is selected from H and alkyl;
R_{E351} is selected from alkyl, -aryl-(C₁-C₄ alkyl), alkyl (optionally substituted with 1, 2, or 3 groups independently selected from halogen, cyano, heteroaryl, —NR_{E6}R_{E7}, —C(O)NR_{E6}R_{E7}, —C₃-C₇ cycloalkyl, and —C₁-C₄ alkoxy), heterocycloalkyl (optionally substituted with 1 or 2 groups independently selected from —C₁-C₄ alkyl, —C₁-C₄ alkoxy, halogen, —C₂-C₄ alkanoyl, -aryl-(C₁-C₄ alkyl), and —SO₂—(C₁-C₄ alkyl)), heteroaryl (optionally substituted with 1, 2, or 3 groups independently selected from —OH, —C₁-C₄ alkyl, —C₁-C₄ alkoxy, halogen, —NH₂, —NH(alkyl), and —N(alkyl)(alkyl)), heteroarylalkyl (optionally substituted with 1, 2, or 3 groups independently selected from —C₁-C₄ alkyl, —C₁-C₄ alkoxy, halogen, —NH₂, —NH(alkyl), and —N(alkyl)(alkyl)), aryl, heterocycloalkyl, —C₃-C₈ cycloalkyl, and cycloalkylalkyl;
wherein the aryl, heterocycloalkyl, —C₃-C₈ cycloalkyl, and cycloalkylalkyl groups included within R_{E351} are optionally substituted with 1, 2, 3, 4 or 5 groups independently selected from halogen, —CN, —NO₂, alkyl, alkoxy, alkanoyl, haloalkyl, haloalkoxy, hydroxy, hydroxyalkyl, alkoxyalkyl, —C₁-C₆ thioalkoxy, —C₁-C₆ thioalkoxy-alkyl, and alkoxyalkoxy;
R_{E352} is selected from heterocycloalkyl, heteroaryl, aryl, cycloalkyl, —S(O)₀₋₂-alkyl, —CO₂H, —C(O)NH₂, —C(O)NH(alkyl), —C(O)N(alkyl)(alkyl), —CO₂-alkyl, —NHS(O)₀₋₂-alkyl, —N(alkyl)S(O)₀₋₂-alkyl, —S(O)₀₋₂-heteroaryl, —S(O)₀₋₂aryl, —NH(arylalkyl), —N(alkyl)(arylalkyl), thioalkoxy, and alkoxy;
wherein each group included within R₃₅₂ is optionally substituted with 1, 2, 3, 4, or 5 groups that are independently selected from alkyl, alkoxy, thioalkoxy, halogen, haloalkyl, haloalkoxy, alkanoyl, —NO₂, —CN, alkoxycarbonyl, and aminocarbonyl;
R_{E353} is selected from —O—, —C(O)—, —NH—, —N(alkyl)-, —NH—S(O)₀₋₂—, —N(alkyl)-S(O)₀₋₂—, —S(O)₀₋₂—NH—, —S(O)₀₋₂—N(alkyl)-, —NH—C(S)—, and —N(alkyl)—C(S)—;
R_{E354} is selected from heteroaryl, aryl, arylalkyl, heterocycloalkyl, —CO₂H, —CO₂-alkyl, —C(O)NH(alkyl), —C(O)N(alkyl)(alkyl), —C(O)NH₂, —C₁-C₈ alkyl, —OH, aryloxy, alkoxy, arylalkoxy, —NH₂, —NH(alkyl), —N(alkyl)(alkyl), and -alkyl-CO₂-alkyl;
wherein each group included within R_{E354} is optionally substituted with 1, 2, 3, 4, or 5 groups that are independently selected from alkyl, alkoxy, —CO₂H, —CO₂-alkyl, thioalkoxy, halogen, haloalkyl, haloalkoxy, hydroxyalkyl, alkanoyl, —NO₂, —CN, alkoxycarbonyl, and aminocarbonyl;
E₁ is selected from —NR_{E11}— and —C₁-C₆ alkyl- (optionally substituted with 1, 2, or 3 groups selected from —C₁-C₄ alkyl), and R_{E11} is selected from —H and alkyl; or R_{E1} and R_{E11} combine to form —(CH₂)₁₋₄-;
E₂ is selected from a bond, —SO₂—, —SO—, —S—, and —C(O)—; and
E₃ is selected from —H, —C₁-C₄ haloalkyl, —C₅-C₆ heterocycloalkyl, —C₆-C₁₀ aryl, —OH, —N(E₃ₐ)(E_{3b}), —C₁-C₁₀ alkyl (optionally substituted with 1, 2, or 3 groups independently selected from halogen, hydroxy, alkoxy, thioalkoxy, and haloalkoxy), —C₃-C₈ cycloalkyl (optionally substituted with 1, 2, or 3 groups independently selected from —C₁-C₃ alkyl and halogen), alkoxy, aryl (optionally substituted with at least one group selected from halogen, alkyl, alkoxy, —CN and —NO₂), arylalkyl (optionally substituted with a group selected from halogen, alkyl, alkoxy, —CN, and —NO₂);
E₃ₐ and E_{3b} are independently selected from —H, —C₁-C₁₀ alkyl (optionally substituted with 1, 2, or 3 groups independently selected from halogen, —C₁-C₄ alkoxy, —C₃-C₈ cycloalkyl, and —OH), —C₂-C₆ alkyl, —C₂-C₆ alkanoyl, aryl, —SO₂—C₁-C₄ alkyl, -aryl-C₁-C₄ alkyl, and —C₃-C₈ cycloalkyl C₁-C₄ alkyl; or
E₃ₐ, E_{3b}, and the nitrogen to which they are attached may optionally form a ring selected from piperazinyl, piperidinyl, morpholinyl, and pyrrolidinyl;
wherein each ring is optionally substituted with 1, 2, 3, or 4 groups that are independently selected from alkyl, alkoxy, alkoxyalkyl, and halogen;
W is selected from —(CH₂)₀₋₄—, —O—, —S(O)₀₋₂—, —N(R₁₃₅)—, —CR(OH)—, and —C(O)—;
R₁₀₂ and R₁₀₂′ are independently selected from hydrogen, —OH, and —C₁-C₁₀ alkyl optionally substituted with 1, 2, or 3 groups independently selected from -halogen, -aryl, and —R₁₁₀;
R₁₀₅ and R′₁₀₅ are independently selected from
—H,
—R₁₁₀,
—R₁₂₀,
-cycloalkyl,
—(C₁-C₂ alkyl)-cycloalkyl,
-(alkyl)-O—(C₁-C₃ alkyl), and
-alkyl optionally substituted with at least one group independently selected from —OH, -amine, and -halogen; or
R₁₀₅ and R′₁₀₅ together with the atom to which they are attached form a 3, 4, 5, 6, or 7 membered carbocyclic ring, wherein one member is optionally a heteroatom selected from —O—, —S(O)₀₋₂—, and —N(R₁₃₅)—, wherein the carbocyclic ring is optionally substituted with 1, 2 or 3 R₁₄₀ groups; and
wherein the at least one carbon of the carbocyclic ring is optionally replaced with —C(O)—;
R₁₁₀ is aryl optionally substituted with 1 or 2 R₁₂₅ groups;
R₁₁₅ at each occurrence is independently selected from halogen, —OH, —C(O)—O—R₁₀₂, —C₁-C₆ thioalkoxy, —C(O)—O-aryl, —NR₁₀₅R′₁₀₅, —SO₂—(C₁-C₈ alkyl), —C(O)—R₁₈₀, R₁₈₀, —C(O)NR₁₀₅R′₁₀₅, —SO₂NR₁₀₅R′₁₀₅, —NH—C(O)-(alkyl), —NH—C(O) —OH, —NH—C(O)—OR, —NH—C(O)—O-aryl, —O—C(O)-(alkyl), —O-C(O)-amino, —O—C(O)-monoalkylamino, —O—C(O)-dialkylamino, —O—C(O)-aryl, —O-(alkyl)-C(O)—O—H, —NH—SO₂-(alkyl), -alkoxy, and -haloalkoxy;
R₁₂₀ is -heteroaryl, optionally substituted with 1 or 2 R₁₂₅ groups;
R₁₂₅ at each occurrence is independently selected from -halogen, -amino, -monoalkylamino, -dialkylamino, —OH, —CN, —SO₂—NH₂, —SO₂—NH-alkyl, —SO₂-N(alkyl)₂, —SO₂—(C₁-C₄ alkyl), —C(O)—NH₂, —C(O)—NH-alkyl, —C(O)—N(alkyl)₂, -alkyl optionally substituted with 1, 2, or 3 groups independently selected from C₁-C₃ alkyl, halogen, —OH, —SH, —CN, —CF₃, —C₁-C3 alkoxy, -amino, -monoalkylamino, and -dialkylamino, and -alkoxy optionally substituted with 1, 2, or 3-halogen;
R₁₃₀ is heterocycloalkyl optionally substituted with 1 or 2 R₁₂₅ groups;
R₁₃₅ is independently selected from alkyl, cycloalkyl, —(CH₂)₀₋₂-(aryl), —(CH₂)₀₋₂-(heteroaryl), and —(CH₂)₀₋₂-(heterocycloalkyl);
R₁₄₀ at each occurrence is independently selected from heterocycloalkyl optionally substituted with 1, 2, 3, or 4 groups independently selected from -alkyl, -alkoxy, -halogen, -hydroxy, -cyano, -nitro, -amino, -monoalkylamino, -dialkylamino, -haloalkyl, -haloalkoxy, -amino-alkyl, -monoalkylamino-alkyl, and -dialkylaminoalkyl; and wherein at least one carbon of the heterocycloalkyl is optionally replaced with —C(O);
R₁₅₀ is independently selected from
-hydrogen,
-cycloalkyl,
—(C₁-C₂ alkyl)-cycloalkyl,
—R₁₁₀,
R₁₂₀, and
-alkyl optionally substituted with 1, 2, 3, or 4 groups independently selected from —OH, —NH₂, —C₁-C₃ alkoxy, —R₁₁₀, and -halogen;
R₁₅₀′ is independently selected from
-cycloalkyl,
—(C₁-C₃ alkyl)-cycloalkyl,
R₁₂₀, and
-alkyl optionally substituted with 1, 2, 3, or 4 groups independently selected from —OH, —NH₂, —C₁-C₃ alkoxy, —R₁₁₀, and -halogen; and
R₁₈₀ is independently selected from
-morpholinyl,
-thiomorpholinyl,
-piperazinyl,
-piperidinyl,
-homomorpholinyl,
-homothiomorpholinyl,
-homothiomorpholinyl S-oxide,
-homothiomorpholinyl S,S-dioxide,
-pyrrolinyl, and
-pyrrolidinyl;
wherein each R₁₈₀ is optionally substituted with 1, 2, 3, or 4 groups independently selected from -alkyl, -alkoxy, -halogen, -hydroxy, -cyano, -nitro, -amino, -monoalkylamino, -dialkylamino, -haloalkyl, -haloalkoxy, -aminoalkyl, -monoalkylamino-alkyl, -dialkylamino-alkyl and —C(O); and
wherein at least one carbon of R₁₈₀ is optionally replaced with —C(O)—;
R_{c} is selected from fused ring of formulae (IIIa) and (IIIb), 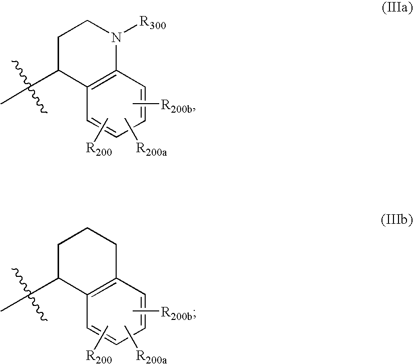
wherein 1, 2, or 3 carbons of the cycloalkyl of formulae (IIIa) and (IIIb) are optionally replaced with —C(O)—, wherein at least one carbon of the fused heterocycloalkyl of IIIa and wherein at least one carbon of the fused cycloalkyl of IIIb is optionally substituted with one or two groups each independently selected from —R₂₀₅, —R₂₄₅, and —R₂₅₀;
R₂₀₀, R₂₀₀ₐ, and R_{200b} at each occurrence are independently selected from:
—H,
-alkyl optionally substituted with at least one group independently selected from R₂₀₅,
—OH,
—NO₂,
-halogen,
—CN,
—(CH₂)₀₋₄—C(O)H,
—(CO)₀₋₁R₂₁₅,
(CO)₀₋₁R₂₂₀,
—(CH₂)₀₋₄—(CO)₀₋₁—NR₂₂₀R₂₂₅,
—(CH₂)₀₋₄—C(O)-alkyl,
—(CH₂)₀₋₄—(CO)₀₋₁-cycloalkyl,
—(CH₂)₀₋₄—(CO)₀₋₁-heterocycloalkyl,
—(CH₂)₀₋₄—(CO)₀₋₁-aryl,
—(CH₂)₀₋₄—(CO)₀₋₁-heteroaryl,
—(CH₂)₀₋₄—CO₂R₂₁₅,
—(CH₂)₀₋₄—SO₂—NR₂₂₀R₂₂₅,
—(CH₂)₀₋₄—S(O)₀₋₂-alkyl,
—(CH₂)₀₋₄—S(O)₀₋₂-cycloalkyl,
—(CH₂)₀₋₄—N(H or R₂₁₅)—CO₂R₂₁₅,
—(CH₂)₀₋₄—N(H or R₂₁₅)—SO₂-R₂₂₀,
—(CH₂)₀₋₄—N(H or R₂₁₅)—C(O)—N(R₂₁₅)₂,
—(CH₂)₀₋₄—N(H or R₂₁₅)—C(O)—R₂₂₀,
—(CH₂)₀₋₄—O—C(O)-alkyl,
—(CH₂)₀₋₄—O—(R₂₁₅),
—(CH₂)₀₋₄—S—(R₂₁₅),
—(CH₂)₀₋₄—O-alkyl optionally substituted with at least one halogen, and -adamantane;
wherein each aryl and heteroaryl group included within R₂₀₀ is optionally substituted with at least one group independently selected from
—R₂₀₅,
—R₂₁₀, and
-alkyl optionally substituted with at least one group independently selected from R₂₀₅ and R₂₁₀;
wherein each cycloalkyl or heterocycloalkyl group included within R₂₀₀ is optionally substituted with at least one group independently selected from R₂₁₀;
R₂₀₅ at each occurrence is independently selected from
-alkyl,
-haloalkoxy,
—(CH₂)₀₋₃-cycloalkyl,
-halogen,
—(CH₂)₀₋₆—OH,
-aryl,
—O-aryl,
—OH,
—SH,
—(CH₂)₀₋₄—C(O)H,
—(CH₂)₀₋₆—CN,
—(CH₂)₀₋₆—C(O)—NR₂₃₅R₂₄₀,
—(CH₂)₀₋₆—C(O)—R₂₃₅,
—(CH₂)₀₋₄—N(H or R₂₁₅)—SO₂—R₂₃₅,
—CF₃,
—CN,
-alkoxy,
-alkoxycarbonyl, and
—NR₂₃₅R₂₄₀;
R₂₁₀ at each occurrence is independently selected from
—OH,
—CN,
—(CH₂)₀₋₄—C(O)H,
-alkyl optionally substituted with at least one group independently selected from R₂₀₅,
—S(O)₂-alkyl,
-halogen,
-alkoxy,
-haloalkoxy,
—NR₂₂₀R₂₂₅,
-cycloalkyl optionally substituted with at least one group independently selected from R₂₀₅,
—C(O)-alkyl,
—S(O)₂—NR₂₃₅R₂₄₀,
—C(O)—NR₂₃₅R₂₄₀, and
—S-alkyl;
R₂₁₅ at each occurrence is independently selected from
-alkyl,
—(CH₂)₀₋₂-cycloalkyl,
—(CH₂)₀₋₂-aryl,
—(CH₂)₀₋₂-heteroaryl,
—(CH₂)₀₋₂-heterocycloalkyl, and
—CO₂—CH₂-aryl;
wherein the aryl groups included within R₂₁₅ are optionally substituted with at least one group independently selected from R₂₀₅ and R₂₁₀; and
wherein the heterocycloalkyl and heteroaryl groups included within R₂₁₅ are optionally substituted with R₂₁₀;
R₂₂₀ and R₂₂₅ at each occurrence are independently selected from
—H,
-alkyl,
—(CH₂)₀₋₄—C(O)H,
—(CH₂)₀₋₄—C(O)-alkyl,
-alkylhydroxy,
-alkoxycarbonyl,
-alkylamino,
—S(O)₂-alkyl,
—C(O)-alkyl optionally substituted with at least one halogen,
—C(O)—NH₂,
—C(O)—NH(alkyl),
—C(O)—N(alkyl)(alkyl),
-haloalkyl,
—(CH₂)₀₋2-cycloalkyl,
-(alkyl)-O-(alkyl),
-aryl,
-heteroaryl, and
-heterocycloalkyl;
wherein the aryl, heteroaryl and heterocycloalkyl groups included within R₂₂₀ and R₂₂₅ are each optionally substituted with at least one group independently selected from R₂₇₀;
R₂₃₅ and R₂₄₀ at each occurrence are independently selected from
—H,
—OH,
—CF₃,
—OCH₃,
—NH—CH₃,
—N(CH₃)₂,
—(CH₂)₀₋₄—C(O)—(H or alkyl),
-alkyl,
—C(O)-alkyl,
—SO₂-alkyl, and
-aryl;
R₂₄₅ and R₂₅₀ at each occurrence are independently selected from
—H,
—OH,
—(CH₂)₀₋₄CO₂-alkyl,
—(CH₂)₀₌₄—C(O)-alkyl,
-alkyl,
-hydroxyalkyl,
-alkoxy,
-haloalkoxy,
—(CH₂)₀₋₄-cycloalkyl,
—(CH₂)₀₋₄-aryl,
—(CH₂)₀₋₄-heteroaryl, and
—(CH₂)₀₋₄-heterocycloalkyl; or
R₂₄₅ and R₂₅₀ are taken together with the carbon to which they are attached to form a monocyclic or bicyclic ring system of 3, 4, 5, 6, 7, or 8 carbon atoms,
wherein at least one carbon atom of the monocyclic or bicyclic ring system is optionally replaced by at least one group independently selected from —O—, —S—, —SO₂—, —C(O)—, —NR₂₂₀—, and —N(alkyl)(alkyl); and
wherein the ring is optionally substituted with at least one group independently selected from
-alkyl,
-alkoxy,
—OH,
—NH₂,
—NH(alkyl),
—N(alkyl)(alkyl),
—NH—C(O)-alkyl,
—NH—SO₂-alkyl, and
-halogen;
wherein the aryl, heteroaryl, or heterocycloalkyl groups included within
R₂₄₅ and R₂₅₀ are optionally substituted with at least one group independently selected from halogen, alkyl, —CN, and —OH;
R₂₇₀ at each occurrence is independently selected from
—R₂₀₅,
-alkyl optionally substituted with at least one group independently selected from R₂₀₅,
-aryl,
-halogen,
-alkoxy,
-haloalkoxy,
—NR₂₃₅R₂₄₀,
—OH,
—CN,
-cycloalkyl optionally substituted with at least one group independently selected from R₂₀₅,
—C(O)-alkyl,
—S(O)₂—NR₂₃₅R₂₄₀,
—CO—NR₂₃₅R₂₄₀,
—S(O)₂-alkyl, and
—(CH₂)₀₋₄—C(O)H;
R₃₀₀ is selected from
—H,
—(CO)₀₋₁R₂₁₅, and
—(CO)₀₋₁R₂₂₀;
wherein at least one carbon of the aryl group of formulae (IIIa) or (IIIb) is optionally replaced by a heteroatom.

2. The method according to claim 1, wherein R₁ is selected from —CH₂-phenyl, wherein the phenyl ring is optionally substituted with at least one group independently selected from halogen, alkyl, alkoxy, and —OH.

3. The method according to claim 1, wherein R₁ is selected from 3-Allyloxy-5-fluoro-benzyl, 3-Benzyloxy-5-fluoro-benzyl, 4-hydroxy-benzyl, 3-hydroxy-benzyl, 3-propyl-thiophen-2-yl-methyl, 3,5-difluoro-2-propylamino-benzyl, 5-chloro-thiopen-2-yl-methyl, 5-chloro-3-ethyl-thiophen-2-yl-methyl, 3,5-difluoro-2-hydroxy-benzyl, 2-ethylamino-3,5-difluoro-benzyl, piperidin-4-yl-methyl, 2-oxo-piperidin-4-yl-methyl, 2-oxo-1,2-dihydro-pyridin-4-yl-methyl, 5-hydroxy-6-oxo-6H-pyran-2-yl-methyl, 2-Hydroxy-5-methyl-benzamide, 3,5-Difluoro-4-hydroxy-benzyl, 3,5-Difluoro-benzyl, 3-Fluoro-4-hydroxy-benzyl, 3-Fluoro-5-[2-(2-methoxy-ethoxy)-ethoxy]-benzyl, 3-Fluoro-5-heptyloxy-benzyl, 3-Fluoro-5-hexyloxy-benzyl, 3-Fluoro-5-hydroxy-benzyl, and 3-Fluoro-benzyl.

4. The method according to claim 1, wherein R₁ is 3,5-difluorobenzyl.

5. The method according to claim 1, wherein R₂ is selected from —C(O)—CH₃ and —C(O)—CH₂F.

6. The method according to claim 1, wherein R₂ is —C(O)—CH₃.

7. The method according to claim 1, wherein R₂ is selected from tert-butyl formate, 2,2-difluoroacetaldehyde, 2-hydroxyacetaldehyde, hydrosulfonylmethane, N-(3-formylphenyl)methanesulfonamide, and N-(3-formylphenyl)-N-methylmethanesulfonamide.

8. The method according to claim 1, wherein U is selected from —C(O)—, —C(S)—, —S(O)₀₋₂—, —C(NR₂₁)—, —C(N—OR₂₁)—, —C(O)—NR₂₀—, —C(O)—O—, —S(O)₂—NR₂₀—, and —S(O)₂—O—; and V is -(T)₀₋₁—R_{N}.

9. The method according to claim 1, wherein U is —C(O)—.

10. The method according to claim 1, wherein U is selected from —C(O)— and —S(O)₀₋₂—; and V is selected from alkyl, alkoxy, aryl, heteroaryl, cycloalkyl, and heterocycloalkyl; wherein the alkyl included within V are optionally substituted with at least one group independently selected from —OH, —NH₂, and halogen; and wherein the aryl, heteroaryl, cycloalkyl, and heterocycloalkyl groups included within V are optionally substituted with 1 or 2 R_{B} groups.

11. The method according to claim 1, wherein U′ is selected from —C(O)—, —C(NR₂₁)—, —C(N—OR₂₁)—, —C(O)—NR₂₀—, and —C(O)—O—; and V′ is -(T)₀₋₁-R_{N′}.

12. The method according to claim 1, wherein R_{N} is selected from alkyl, —(CH₂)₀₋₂-aryl, C₂-C₆ alkyl, C₃-C₇ cycloalkyl, —(CH₂)₀₋₂-heteroaryl, and 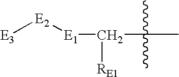
wherein
E₁ is selected from —NR _{E11}— and C₁-C₆ alkyl optionally substituted with 1, 2, or 3 C₁-C₄ groups, R_{E1} is —NH₂, and R_{E11} is selected from —H and alkyl, or R_{E1} and R_{E11} combine to form —(CH₂)₁₋₄—,
E₂ is selected from a bond; SO₂, SO, S, and C(O);
E₃ is selected from
—H,
—C₁-C₄ haloalkyl,
—C₅-C₆ heterocycloalkyl containing at least one N, O, or S,
-aryl,
—OH,
—N(E₃ₐ)(E_{3b}),
—C₁-C₁₀ alkyl optionally substituted with 1, 2, or thru 3 groups which can be the sameindependently or different and are se selected from halogen, hydroxy, alkoxy, thioalkoxy, and haloalkoxy,
—C₃-C₈ cycloalkyl optionally substituted with 1, 2, or 3 groups independently selected from C₁-C₃ alkyl, and halogen,
-alkoxy,
-aryl optionally substituted with at least one group selected from halogen, C₁-C₄ alkyl, C₁-C₄ alkoxy, —CN, and -NO₂ and
-aryl C₁-C₄ alkyl optionally substituted with at least one group selected from halogen, C₁-C₄ alkyl, C₁-C₄ alkoxy, —CN, and —NO₂,
E₃ₐ and E_{3b} are independently selected from
—H,
—C₁-C₁₀ alkyl optionally substituted with 1, 2, or 3 groups independently selected from halogen, C₁-C₄ alkoxy, C₃-C₈ cycloalkyl, and —OH,
—C₂-C₆ alkanoyl,
-aryl,
—SO₂—C₁-C₄ alkyl,
-aryl C₁-C₄ alkyl, and
—C₃-C₈ cycloalkyl C₁-C₄ alkyl, or
E₃ₐ, E_{3b}, and the nitrogen to which they are attached form a ring selected from piperazinyl, piperidinyl, morpholinyl, and pyrolidinyl, wherein each ring is optionally substituted with 1, 2, 3, or 4 groups that are independently selected from alkyl, alkoxy, alkoxyalkyl, and halogen.

13. The method according to claim 1, wherein
V is —(CH₂)₁₋₃-aryl or —(CH₂)₁₋₃-heteroaryl, wherein each ring is independently optionally substituted with 1 or 2 groups independently selected from halogen, —OH, —OCF₃, —O-aryl, —CN, —NR₁₀₁R′₁₀₁, alkyl, alkoxy, (CH₂)₀₋₃(C₃-C₇ cycloalkyl), aryl, heteroaryl, and heterocycloalkyl, and wherein
the alkyl, alkoxy, cycloalkyl, aryl, heteroaryl, or heterocycloalkyl groups are optionally substituted with 1 or 2 groups independently selected from C₁-C₄ alkyl, C₁-C₄ alkoxy, C₁-C₄ haloalkyl, C₁-C₄ haloalkoxy, halogen, —OH, —CN, and —NR₁₀₁R′₁₀₁.

14. The method according to claim 1, wherein R_{c} is selected from
7-(4-methyl-thiophen-3-yl)-1,2,3,4-tetrahydro-naphthalen-1-yl, 7-(3-methyl-3H-imidazol-4-yl)-1,2,3,4-tetrahydro-naphthalen-1-yl, 7-(4-methyl-pyridin-2-yl)-1,2,3,4-tetrahydro-naphthalen-1-yl, 7-pyrimidin-2-yl-1,2,3,4-tetrahydro-naphthalen-1-yl, 7-isopropenyl-1,2,3,4-tetrahydro-naphthalen-1-yl, 7-(4-trifluoromethyl-pyrimidin-2-yl)-1,2,3,4-tetrahydro-naphthalen-1-yl, 7-(2-methylsulfanyl-pyrimidin-4-yl)-1,2,3,4-tetrahydro-naphthalen-1-yl, 7-pyrimidin-5-yl-1,2,3,4-tetrahydro-naphthalen-1-yl, 7-pyridin-2-yl-1,2,3,4-tetrahydro-naphthalen-1-yl, 7-(5-methyl-pyridin-2-yl)-1,2,3,4-tetrahydro-naphthalen-1-yl, 7-pyridin-3-yl-1,2,3,4-tetrahydro-naphthalen-1-yl, 7-(3-methyl-pyridin-2-yl)-1,2,3,4-tetrahydro-naphthalen-1-yl, 7-(6-methyl-pyridazin-3-yl)-1,2,3,4-tetrahydro-naphthalen-1-yl, 7-pyridin-4-yl-1,2,3,4-tetrahydro-naphthalen-1-yl, 7-(6-methyl-pyridin-3-yl)-1,2,3,4-tetrahydro-naphthalen-1-yl, 7-(6-methoxy-pyridazin-3-yl)-1,2,3,4-tetrahydro-naphthalen-1-yl, 7-(4-methyl-pyridin-3-yl)-1,2,3,4-tetrahydro-naphthalen-1-yl, 7-pyrazin-2-yl-1,2,3,4-tetrahydro-naphthalen-1-yl, 7-(5-methyl-thiophen-2-yl)-1,2,3,4-tetrahydro-naphthalen-1-yl, 7-thiazol-2-yl-1,2,3,4-tetrahydro-naphthalen-1-yl, 7-thiophen-3-yl-1,2,3,4-tetrahydro-naphthalen-1-yl, 7-(1-methyl-1H-imidazol-2-yl)-1,2,3,4-tetrahydro-naphthalen-1-yl, 7-thiophen-2-yl-1,2,3,4-tetrahydro-naphthalen-1-yl, 7-(3-methyl-thiophen-2-yl)-1,2,3,4-tetrahydro-naphthalen-1-yl, 5-(3-Amino-phenyl)-7-ethyl-1,2,3,4-tetrahydro-naphthalen-1-yl, 7-ethyl-5-thiazol-2-yl-1,2,3,4-tetrahydro-naphthalen-1-yl, 7-ethyl-5-pyridin-2-yl-1,2,3,4-tetrahydro-naphthalen-1-yl, 7-ethyl-5-(3-methyl-pyridin-2-yl)-1,2,3,4-tetrahydro-naphthalen-1-yl, 7-ethyl-5-(4-methyl-pyridin-2-yl)-1,2,3,4-tetrahydro-naphthalen-1-yl, 7-(2,2-dimethyl-propyl)-1,2,3,4-tetrahydro-naphthalen-1-yl, 6-(2,2-dimethyl-propyl)-1-methyl-1,2,3,4-tetrahydro-quinolin-4-yl, 7-(2,2-dimethyl-propyl)-4-oxo-1,2,3,4-tetrahydro-naphthalen-1-yl, 7-(2,2-dimethyl-propyl)-5-ethyl-1,2,3,4-tetrahydro-naphthalen-1-yl, 6-(2,2-dimethyl-propyl)-1,2,3,4-tetrahydro-quinolin-4-yl, 7-(2,2-dimethyl-propyl)-1-methyl-1,2,3,4-tetrahydro-naphthalen-1-yl, 7-propyl-1,2,3,4-tetrahydro-naphthalen-1-yl, 6-isopropyl-2-oxo-1,2,3,4-tetrahydro-quinolin-4-yl, 7-isopropyl-3-oxo-1,2,3,4-tetrahydro-naphthalen-1-yl, 3-hydroxy-7-isopropyl-3-methyl-1,2,3,4-tetrahydro-naphthalen-1-yl, 3-Acetylamino-7-isopropyl-1,2,3,4-tetrahydro-naphthalen-1-yl, 7-isopropyl-3-methanesulfonylamino-1,2,3,4-tetrahydro-naphthalen-1-yl, 1,2,3,4-tetrahydro-naphthalen-1-yl, 7-methoxy-1,2,3,4-tetrahydro-naphthalen-1-yl, 7-ethyl-1,2,3,4-tetrahydro-naphthalen-1-yl, 6-ethyl-1-methyl-1,2,3,4-tetrahydro-quinolin-4-yl, 7-dimethylaminomethyl-1,2,3,4-tetrahydro-naphthalen-1-yl, 7-Bromo-1,2,3,4-tetrahydro-naphthalen-1-yl, 6-carbobenzoxy-1,2,3,4-tetrahydro-quinolin-4-yl, 7-ethyl-2,2-yl, dimethyl-1,2,3,4-tetrahydro-naphthalen-1-yl, 7-isobutyl-1,2,3,4-tetrahydro-naphthalen-1-5-Bromo-7-ethyl-1,2,3,4-tetrahydro-naphthalen-1-yl, 5,7-Diethyl-1,2,3,4-tetrahydro-naphthalen-1-yl, 5-Butyl-7-ethyl-1,2,3,4-tetrahydro-naphthalen-1-yl, 7-ethyl-5-propyl-1,2,3,4-tetrahydro-naphthalen-1-yl, 7-ethyl-5-isobutyl-1,2,3,4-tetrahydro-naphthalen-1-yl, 7-(2,2-dimethyl-propyl)-2-hydroxymethyl-1,2,3,4-tetrahydro-naphthalen-1-yl, 7-ethyl-5-(5-methyl-pyridin-2-yl)-1,2,3,4-tetrahydro-naphthalen-1-yl, 7-ethyl-5-(6-methyl-pyridin-2-yl)-1,2,3,4-tetrahydro-naphthalen-1-yl, 7-Butyl-1,2,3,4-tetrahydro-naphthalen-1-yl, 5-Cyano-7-ethyl-1,2,3,4-tetrahydro-naphthalen-1-yl, 6-ethyl-1,2,3,4-tetrahydro-quinolin-4-yl, 7-ethyl-1-methyl-1,2,3,4-tetrahydro-naphthalen-1-yl, 7-sec-Butyl-1,2,3,4-tetrahydro-naphthalen-1-yl, 2-hydroxy-6-neopentyl-1,2,3,4-tetrahydroquinolin-4-yl, 2-hydroxy-6-isobutyl-1,2,3,4-tetrahydroquinolin-4-yl, 2-hydroxy-6-isopropyl-1,2,3,4-tetrahydroquinolin-4-yl, 6-tert-butyl-1,2,3,4-tetrahydroquinolin-4-yl, 6-ethyl-1,2,3,4-tetrahydroquinolin-4-yl, 7-fluoro-6-isopropyl-1,2,3,4-tetrahydroquinolin-4-yl, 6-tert-butyl-7-fluoro-1,2,3,4-tetrahydroquinolin-4-yl, 7-fluoro-6-isobutyl-1,2,3,4-tetrahydroquinolin-4-yl, 7-fluoro-6-neopentyl-1,2,3,4-tetrahydroquinolin-4-yl, 2-hydroxy-1-methyl-6-neopentyl-1,2,3,4-tetrahydroquinolin-4-yl, 2-hydroxy-6-isobutyl-1-methyl-1,2,3,4-tetrahydroquinolin-4-yl, 2-hydroxy-6-isopropyl-1-methyl-1,2,3,4-tetrahydroquinolin-4-yl,6-tert-butyl-1-methyl-1,2,3,4-tetrahydroquinolin-4-yl, 6-tert-butyl-1-(2-hydroxyethyl)-1,2,3,4-tetrahydroquinolin-4-yl, 2-hydroxy-1-(2-hydroxyethyl)-6-isopropyl-1,2,3,4-tetrahydroquinolin-4-yl, 2-hydroxy-1-(2-hydroxyethyl)-6-isobutyl-1,2,3,4-tetrahydroquinolin-4-yl, 2-hydroxy-1-(2-hydroxyethyl)-6-neopentyl-1,2,3,4-tetrahydroquinolin-4-yl, 1-acetyl-6-neopentyl-1,2,3,4-tetrahydroquinolin-4-yl, 1-acetyl-6-isobutyl-1,2,3,4-tetrahydroquinolin-4-yl, 1-acetyl-6-isopropyl-1,2,3,4-tetrahydroquinolin-4-yl, 1-acetyl-6-tert-butyl-1,2,3,4-tetrahydroquinolin-4-yl, 6-tert-butyl-1-(cyanomethyl)-1,2,3,4-tetrahydroquinolin-4-yl, 1-(cyanomethyl)-6-isopropyl-1,2,3,4-tetrahydroquinolin-4-yl, 1-(cyanomethyl)-6-isobutyl-1,2,3,4-tetrahydroquinolin-4-yl, 1-(cyanomethyl)-6-neopentyl-1,2,3,4-tetrahydroquinolin-4-yl, 2-hydroxy-6-(1-hydroxy-2,2-dimethylpropyl)-1,2,3,4-tetrahydroquinolin-4-yl, 2-hydroxy-6-(1-hydroxy-2,2-dimethylpropyl)-1-methyl-1,2,3,4-tetrahydroquinolin-4-yl, 2,2-dimethyl-6-neopentyl-1,2,3,4-tetrahydroquinolin-4-yl,2-hydroxy-1,2,2-trimethyl-6-neopentyl-1,2,3,4-tetrahydroquinolin-4-yl, 1,4-dimethyl-6-neopentyl-1,2,3,4-tetrahydroquinolin-4-yl, 2-hydroxy-4-methyl-6-neopentyl-1,2,3,4-tetrahydroquinolin-4-yl, 2-hydroxy-6-isobutyl-4-methyl-1,2,3,4-tetrahydroquinolin-4-yl, 2-hydroxy-6-isobutyl-1,4-dimethyl-1,2,3,4-tetrahydroquinolin-4-yl, 6-tert-butoxy-1,2,3,4-tetrahydroquinolin-4-yl, 6-tert-butoxy-4-methyl-1,2,3,4-tetrahydroquinolin-4-yl, 6-tert-butoxy-4,8-dimethyl-1,2,3,4-tetrahydroquinolin-4-yl, 2-hydroxy-4-methyl-6-neopentyl-1,2,3,4-tetrahydroquinolin-4-yl, 4,8-dimethyl-6-neopentyl-1,2,3,4-tetrahydroquinolin-4-yl, 2-hydroxy-8-methyl-6-neopentyl-1,2,3,4-tetrahydroquinolin-4-yl, 2-hydroxy-6-(2-hydroxy-2-methylpropyl)-8-methyl-1,2,3,4-tetrahydroquinolin-4-yl, 2-hydroxy-6-(2-hydroxy-2-methylpropyl)-4-methyl-1,2,3,4-tetrahydroquinolin-4-yl, 2-hydroxy-6-(2-hydroxy-2-methylpropyl)-1,2,3,4-tetrahydroquinolin-4-yl, 2-hydroxy-6-(1-hydroxy-2,2-dimethylpropyl)-1,2,3,4-tetrahydroquinolin-4-yl, 2-hydroxy-6-(1-hydroxy-2,2-dimethylpropyl)-4-methyl-1,2,3,4-tetrahydroquinolin-4-yl, 2-hydroxy-5-isobutyl-2-pyridin-3-ylbenzyl, 2-hydroxy-5-isobutyl-2-pyridin-4-ylbenzyl, 2-hydroxy-5-isobutyl-2-(6-methoxypyridin-3-yl)benzyl, 2-hydroxy-5-isobutyl-2-(5-methoxypyridin-3-yl)benzyl, 5,7-Diethyl-1,2,3,4-tetrahydronaphthalen-1-yl, 7-ethyl-5-propyl-1,2,3,4-tetrahydro-naphthalen-1-yl, 7-ethyl-5-isobutyl-1,2,3,4-tetrahydronaphthalen-1-yl, 7-(3,6-dimethyl-pyrazin-2-yl)-1,2,3,4-tetrahydro-naphthalen-1-yl, 7-furan-2-yl-1,2,3,4-tetrahydro-naphthalen-1-yl, 7-styryl-1,2,3,4-tetrahydro-naphthalen-1-yl, 7-(3,5-dimethyl-isoxazol-4-yl)-1,2,3,4-tetrahydro-naphthalen-1-yl, 7-(5-ethyl-pyrimidin-2-yl)-1,2,3,4-tetrahydro-naphthalen-1-yl, 1-[3-(5-Acetyl-thiophen-2-yl)-phenyl]-cyclopropyl, 1-(3-thiophen-3-yl-phenyl)-cyclopropyl, 1-[3-(6-methoxy-pyridin-3-yl)-phenyl]-cyclopropyl, 1-(3-furan-3-yl-phenyl)-cyclopropyl, 1-[3-(3,5-dimethyl-isoxazol-4-yl)-phenyl]-cyclopropyl, and 5-(3-aminophenyl)-7-ethyl-1,2,3,4-tetrahydronaphthalen-1-yl.

15. The method according to claim 1, wherein the at least one compound of formula (I) is chosen from
N-{1-(3,5-Difluoro-benzyl)-2-hydroxy-3-[7-(4-methyl-thiophen-3-yl)-1,2,3,4-tetrahydro-naphthalen-1-ylamino]-propyl}-acetamide, N-{1-(3,5-Difluoro-benzyl)-2-hydroxy-3-[7-(3-methyl-3H-imidazol-4-yl)-1,2,3,4-tetrahydro-naphthalen-1-ylamino]-propyl}-acetamide, N-{1-(3,5-Difluoro-benzyl)-2-hydroxy-3-[7-(4-methyl-pyridin-2-yl)-1,2,3,4-tetrahydro-naphthalen-1-ylamino]-propyl}-acetamide, N-[1-(3,5-Difluoro-benzyl)-2-hydroxy-3-(7-pyrimidin-2-yl-1,2,3,4-tetrahydro-naphthalen-1-ylamino)-propyl]-acetamide, N-[1-(3,5-Difluoro-benzyl)-2-hydroxy-3-(7-isopropenyl-1,2,3,4-tetrahydro-naphthalen-1-ylamino)-propyl]-acetamide, N-{1-(3,5-Difluoro-benzyl)-2-hydroxy-3-[7-(4-trifluoromethyl-pyrimidin-2-yl)-1,2,3,4-tetrahydro-naphthalen-1-ylamino]-propyl}-acetamide, N-{1-(3,5-Difluoro-benzyl)-2-hydroxy-3-[7-(2-methylsulfany-pyrimidin-4-yl)-1,2,3,4-tetrahydro-naphthalen-1-ylamino]-propyl}-acetamide, N-[1-(3,5-Difluoro-benzyl)-2-hydroxy-3-(7-pyrimidin-5-yl-1,2,3,4-tetrahydro-naphthalen-1-ylamino)-propyl]-acetamide, N-[1-(3,5-Difluoro-benzyl)-2-hydroxy-3-(7-pyridin-2-yl-1,2,3,4-tetrahydro-naphthalen-1-ylamino)-propyl]-acetamide, N-{1-(3,5-Difluoro-benzyl)-2-hydroxy-3-[7-(5-methyl-pyridin-2-yl)-1,2,3,4-tetrahydro-naphthalen-1-ylamino]-propyl}-acetamide, N-[1-(3,5-Difluoro-benzyl)-2-hydroxy-3-(7-pyridin-3-yl-1,2,3,4-tetrahydro-naphthalen-1-ylamino)-propyl]-acetamide, N-{1-(3,5-Difluoro-benzyl)-2-hydroxy-3-[7-(3-methyl-pyridin-2-yl)-1,2,3,4-tetrahydro-naphthalen-1-ylamino]-propyl}-acetamide, N-{1-(3,5-Difluoro-benzyl)-2-hydroxy-3-[7-(6-methyl-pyridazin-3-yl)-1,2,3,4-tetrahydro-naphthalen-1-ylamino]-propyl}-acetamide, N-[1-(3,5-Difluoro-benzyl)-2-hydroxy-3-(7-pyridin-4-yl-1,2,3,4-tetrahydro-naphthalen-1-ylamino)-propyl]-acetamide, N-{1-(3,5-Difluoro-benzyl)-2-hydroxy-3-[7-(6-methyl-pyridin-3-yl)-1,2,3,4-tetrahydro-naphthalen-1-ylamino]-propyl}-acetamide, N-{1-(3,5-Difluoro-benzyl)-2-hydroxy-3-[7-(6-methoxy-pyridazin-3-yl)-1,2,3,4-tetrahydro-naphthalen-1-ylamino]-propyl}-acetamide, N-{1-(3,5-Difluoro-benzyl)-2-hydroxy-3-[7-(4-methyl-pyridin-3-yl)-1,2,3,4-tetrahydro-naphthalen-1-ylamino]-propyl}-acetamide, N-[1-(3,5-Difluoro-benzyl)-2-hydroxy-3-(7-pyrazin-2-yl-1,2,3,4-tetrahydro-naphthalen-1-ylamino)-propyl]-acetamide, N-{1-(3,5-Difluoro-benzyl)-2-hydroxy-3-[7-(5-methyl-thiophen-2-yl)-1,2,3,4-tetrahydro-naphthalen-1-ylamino]-propyl}-acetamide, N-[1-(3,5-Difluoro-benzyl)-2-hydroxy-3-(7-thiazol-2-yl-1,2,3,4-tetrahydro-naphthalen-1-ylamino)-propyl]-acetamide, N-[1-(3,5-Difluoro-benzyl)-2-hydroxy-3-(7-thiophen-3-yl-1,2,3,4-tetrahydro-naphthalen-1-ylamino)-propyl]-acetamide, N-{1-(3,5-Difluoro-benzyl)-2-hydroxy-3-[7-(1-methyl-1H-imidazol-2-yl)-1,2,3,4-tetrahydro-naphthalen-1-ylamino]-propyl}-acetamide, N-[1-(3,5-Difluoro-benzyl)-2-hydroxy-3-(7-thiophen-2-yl-1,2,3,4-tetrahydro-naphthalen-1-ylamino)-propyl]-acetamide, N-{1-(3,5-Difluoro-benzyl)-2-hydroxy-3-[7-(3-methyl-thiophen-2-yl)-1,2,3,4-tetrahydro-naphthalen-1-ylamino]-propyl}-acetamide, N-[3-[5-(3-Amino-phenyl)-7-ethyl-1,2,3,4-tetrahydro-naphthalen-1-ylamino]-1-(3,5-difluoro-benzyl)-2-hydroxy-propyl]-acetamide, N-[1-(3,5-Difluoro-benzyl)-3-(7-ethyl-5-thiazol-2-yl-1,2,3,4-tetrahydro-naphthalen-1-ylamino)-2-hydroxy-propyl]-acetamide, N-[1-(3,5-Difluoro-benzyl)-3-(7-ethyl-5-pyridin-2-yl-1,2,3,4-tetrahydro-naphthalen-1-ylamino)-2-hydroxy-propyl]-acetamide, N-{1-(3,5-Difluoro-benzyl)-3-[7-ethyl-5-(3-methyl-pyridin-2-yl)-1,2,3,4-tetrahydro-naphthalen-1-ylamino]-2-hydroxy-propyl}-acetamide, N-{1-(3,5-Difluoro-benzyl)-3-[7-ethyl-5-(4-methyl-pyridin-2-yl)-1,2,3,4-tetrahydro-naphthalen-1-ylamino]-2-hydroxy-propyl}-acetamide, N-{1-(4-Benzyloxy-3-fluoro-benzyl)-3-[7-(2,2-dimethyl-propyl)-1,2,3,4-tetrahydro-naphthalen-1-ylamino]-2-hydroxy-propyl}-acetamide, N-[3-[7-(2,2-Dimethyl-propyl)-1,2,3,4-tetrahydro-naphthalen-1-ylamino]-1-(3-fluoro-4-hydroxy-benzyl)-2-hydroxy-propyl]-acetamide, N-{1-(3,5-Difluoro-benzyl)-3-[6-(2,2-dimethyl-propyl)-1-methyl-1,2,3,4-tetrahydro-quinolin-4-ylamino]-2-hydroxy-propyl}-acetamide, N-{1-(3,5-Difluoro-benzyl)-3-[7-(2,2-dimethyl-propyl)-4-oxo-1,2,3,4-tetrahydro-naphthalen-1-ylamino]-2-hydroxy-propyl}-acetamide, N-{1-(3,5-Difluoro-benzyl)-3-[7-(2,2-dimethyl-propyl)-5-ethyl-1,2,3,4-tetrahydro-naphthalen-1-ylamino]-2-hydroxy-propyl}-acetamide, N-{1-(3,5-Difluoro-benzyl)-3-[7-(2,2-dimethyl-propyl)-1,2,3,4-tetrahydro-naphthalen-1-ylamino]-2-hydroxy-propyl}-acetamide, N-{1-(3,5-Difluoro-benzyl)-3-[6-(2,2-dimethyl-propyl)-1,2,3,4-tetrahydro-quinolin-4-ylamino]-2-hydroxy-propyl}-acetamide, N-{1-(3,5-Difluoro-benzyl)-3-[7-(2,2-dimethyl-propyl)-1-methyl-1,2,3,4-tetrahydro-naphthalen-1-ylamino]-2-hydroxy-propyl}-acetamide, N-{1-(3,5-Difluoro-benzyl)-3-[7-(2,2-dimethyl-propyl)-1,2,3,4-tetrahydro-naphthalen-1-ylamino]-2-hydroxy-propyl}-2-fluoro-acetamide, N-[1-(3,5-Difluoro-benzyl)-2-hydroxy-3-(7-propyl-1,2,3,4-tetrahydro-naphthalen-1-ylamino)-propyl]acetamide, N-[1-(3,5-Difluoro-benzyl)-2-hydroxy-3-(6-isopropyl-2-oxo-1,2,3,4-tetrahydro-quinolin-4-ylamino)-propyl]-acetamide, N-[1-(3,5-Difluoro-benzyl)-2-hydroxy-3-(7-isopropyl-3-oxo-1,2,3,4-tetrahydro-naphthalen-1-ylamino)-propyl]-acetamide, N-[1-(3,5-Difluoro-benzyl)-2-hydroxy-3-(3-hydroxy-7-isopropyl-3-methyl-1,2,3,4-tetrahydro-naphthalen-1-ylamino)-propyl]-acetamide, N-[3-(3-Acetylamino-7-isopropyl-1,2,3,4-tetrahydro-naphthalen-1-ylamino)-1-(3,5-difluoro-benzyl)-2-hydroxy-propyl]-acetamide, N-[1-(3,5-Difluoro-benzyl)-2-hydroxy-3-(7-isopropyl-3-methanesulfonylamino-1,2,3,4-tetrahydro-naphthalen-1-ylamino)-propyl]-acetamide, N-[3-[7-(2,2-Dimethyl-propyl)-1,2,3,4-tetrahydro-naphthalen-1-ylamino]-2-hydroxy-1-(5-hydroxy-pyridin-2-ylmethyl)-propyl]-acetamide, N-[1-(3,5-Difluoro-benzyl)-2-hydroxy-3-(1,2,3,4-tetrahydro-naphthalen-1-ylamino)-propyl]-acetamide, N-[1-(3,5-Difluoro-benzyl)-2-hydroxy-3-(7-methoxy-1,2,3,4-tetrahydro-naphthalen-1-ylamino)-propyl]-acetamide, N-[1-(3,5-Difluoro-benzyl)-3-(7-ethyl-1,2,3,4-tetrahydro-naphthalen-1-ylamino)-2-hydroxy-propyl]-acetamide, N-[1-(3,5-Difluoro-benzyl)-3-(6-ethyl-1-methyl-1,2,3,4-tetrahydro-quinolin-4-ylamino)-2-hydroxy-propyl]-acetamide, N-[1-(3,5-Difluoro-benzyl)-3-(7-dimethylaminomethyl-1,2,3,4-tetrahydro-naphthalen-1-ylamino)-2-hydroxy-propyl]-acetamide, N-[3-(7-Bromo-1,2,3,4-tetrahydro-naphthalen-1-ylamino)-1-(3,5-difluoro-benzyl)-2-hydroxy-propyl]-acetamide, N-{1-(3,5-Difluoro-benzyl)-3-[6-carbobenzoxy-1,2,3,4-tetrahydro-quinolin-4-ylamino]-2-hydroxy-propyl}-acetamide, N-[1-(3,5-Difluoro-benzyl)-3-(7-ethyl-2,2-dimethyl-1,2,3,4-tetrahydro-naphthalen-1-ylamino)-2-hydroxy-propyl]-acetamide, N-[1-(3,5-Difluoro-benzyl)-2-hydroxy-3-(7-isobutyl-1,2,3,4-tetrahydro-naphthalen-1-ylamino)-propyl]-acetamide, N-[3-(5-Bromo-7-ethyl-1,2,3,4-tetrahydro-naphthalen-1-ylamino)-1-(3,5-difluoro-benzyl)-2-hydroxy-propyl]-acetamide, N-[3-(5,7-Diethyl-1,2,3,4-tetrahydro-naphthalen-1-ylamino)-1-(3,5-difluoro-benzyl)-2-hydroxy-propyl]-acetamide, N-[3-(5-Butyl-7-ethyl-1,2,3,4-tetrahydro-naphthalen-1-ylamino)-1-(3,5-difluoro-benzyl)-2-hydroxy-propyl]-acetamide, N-[1-(3-Butoxy-5-fluoro-benzyl)-3-(7-ethyl-1,2,3,4-tetrahydro-naphthalen-1-ylamino)-2-hydroxy-propyl]-acetamide, N-[1-(3-Benzyloxy-5-fluoro-benzyl)-3-(7-ethyl-1,2,3,4-tetrahydro-naphthalen-1-ylamino)-2-hydroxy-propyl]-acetamide, N-[3-(7-Ethyl-1,2,3,4-tetrahydro-naphthalen-1-ylamino)-1-(3-fluoro-5-hydroxy-benzyl)-2-hydroxy-propyl]-acetamide, N-[1-(3,5-Difluoro-benzyl)-3-(7-ethyl-5-propyl-1,2,3,4-tetrahydro-naphthalen-1-ylamino)-2-hydroxy-propyl]-acetamide, N-[1-(3,5-Difluoro-benzyl)-3-(7-ethyl-5-isobutyl-1,2,3,4-tetrahydro-naphthalen-1-ylamino)-2-hydroxy-propyl]-acetamide, N-{1-(3,5-Difluoro-benzyl)-3-[7-(2,2-dimethyl-propyl)-2-hydroxymethyl-1,2,3,4-tetrahydro-naphthalen-1-ylamino[-2-hydroxy-propyl}-acetamide, N-{1-(3,5-Difluoro-benzyl)-3-[7-ethyl-5-(5-methyl-pyridin-2-yl)-1,2,3,4-tetrahydro-naphthalen-1-ylamino]-2-hydroxy-propyl}-acetamide, N-{1-(3,5-Difluoro-benzyl)-3-[7-ethyl-5-(6-methyl-pyridin-2-yl)-1,2,3,4-tetrahydro-naphthalen-1-ylamino]-2-hydroxy-propyl}-acetamide, N-[3-(7-Butyl-1,2,3,4-tetrahydro-naphthalen-1-ylamino)-1-(3,5-difluoro-benzyl)-2-hydroxy-propyl]-acetamide, N-[3-(5-Cyano-7-ethyl-1,2,3,4-tetrahydro-naphthalen-1-ylamino)-1-(3,5-difluoro-benzyl)-2-hydroxy-propyl]-acetamide, N-[1-(3,5-Difluoro-benzyl)-3-(6-ethyl-1,2,3,4-tetrahydro-quinolin-4-ylamino)-2-hydroxy-propyl]-acetamide, N-[1-(3,5-Difluoro-benzyl)-3-(7-ethyl-1-methyl-1,2,3,4-tetrahydro-naphthalen-1-ylamino)-2-hydroxy-propyl]-acetamide, N-[3-(7-sec-Butyl-1,2,3,4-tetrahydro-naphthalen-1-ylamino)-1-(3,5-difluoro-benzyl)-2-hydroxy-propyl]-acetamide, N-(1-(3,5-difluorobenzyl)-2-hydroxy-3-{[6-neopentyl-1,2,3,4-tetrahydroquinolin-4-yl]amino}propyl)acetamide, N-(1-(3,5-difluorobenzyl)-2-hydroxy-3-{[6-isobutyl-1,2,3,4-tetrahydroquinolin-4-yl]amino}propyl)acetamide, N-(1-(3,5-difluorobenzyl)-2-hydroxy-3-{[6-isopropyl-1,2,3,4-tetrahydroquinolin-4-yl]amino}propyl)acetamide, N-[3-{[6-tert-butyl-1,2,3,4-tetrahydroquinolin-4-yl]amino}-1-(3,5-difluorobenzyl)-2-hydroxypropyl]acetamide, N-(1-(3,5-difluorobenzyl)-3-{[6-ethyl-1,2,3,4-tetrahydroquinolin-4-yl]amino}-2-hydroxypropyl)acetamide, N-(1-(3,5-difluorobenzyl)-3-{[7-fluoro-6-isopropyl-1,2,3,4-tetrahydroquinolin-4-yl]amino}-2-hydroxypropyl)acetamide, N-[3-{[6-tert-butyl-7-fluoro-1,2,3,4-tetrahydroquinolin-4-yl]amino}-1-(3,5-difluorobenzyl)-2-hydroxypropyl]acetamide, N-(1-(3,5-difluorobenzyl)-3-{[7-fluoro-6-isobutyl-1,2,3,4-tetrahydroquinolin-4-yl]amino}-2-hydroxypropyl)acetamide, N-(1-(3,5-difluorobenzyl)-3-{[7-fluoro-6-neopentyl-1,2,3,4-tetrahydroquinolin-4-yl]amino}-2-hydroxypropyl)acetamide, N-(1-(3,5-difluorobenzyl)-2-hydroxy-3-{[1-methyl-6-neopentyl-1,2,3,4-tetrahydroquinolin-4-yl]amino}propyl)acetamide, N-(1-(3,5-difluorobenzyl)-2-hydroxy-3-{[6-isobutyl-1-methyl-1,2,3,4-tetrahydroquinolin-4-yl]amino}propyl)acetamide, N-(1-(3,5-difluorobenzyl)-2-hydroxy-3-{[6-isopropyl-1-methyl-1,2,3,4-tetrahydroquinolin-4-yl]amino}propyl)acetamide, N-[3-{[6-tert-butyl-1-methyl-1,2,3,4-tetrahydroquinolin-4-yl]amino}-1-(3,5-difluorobenzyl)-2-hydroxypropyl]acetamide, N-[3-{[6-tert-butyl-1-(2-hydroxyethyl)-1,2,3,4-tetrahydroquinolin-4-yl]amino}-1-(3,5-difluorobenzyl)-2-hydroxypropyl]acetamide, N-(1-(3,5-difluorobenzyl)-2-hydroxy-3-{[1-(2-hydroxyethyl)-6-isopropyl-1,2,3,4-tetrahydroquinolin-4-yl]amino}propyl)acetamide, N-(1-(3,5-difluorobenzyl)-2-hydroxy-3-{[1-(2-hydroxyethyl)-6-isobutyl-1,2,3,4-tetrahydroquinolin-4-yl]amino}propyl)acetamide, N-(1-(3,5-difluorobenzyl)-2-hydroxy-3-{[1-(2-hydroxyethyl)-6-neopentyl-1,2,3,4-tetrahydroquinolin-4-yl]amino}propyl)acetamide, N-[3-{[1-acetyl-6-neopentyl-1,2,3,4-tetrahydroquinolin-4-yl]amino}-1-(3,5-difluorobenzyl)-2-hydroxypropyl]acetamide, N-[3-{[1-acetyl-6-isobutyl-1,2,3,4-tetrahydroquinolin-4-yl]amino}-1-(3,5-difluorobenzyl)-2-hydroxypropyl]acetamide, N-[3-{[1-acetyl-6-isopropyl-1,2,3,4-tetrahydroquinolin-4-yl]amino}-1-(3,5-difluorobenzyl)-2-hydroxypropyl]acetamide, N-[3-{[1-acetyl-6-tert-butyl-1,2,3,4-tetrahydroquinolin-4-yl]amino}-1-(3,5-difluorobenzyl)-2-hydroxypropyl]acetamide, N-[3-{[6-tert-butyl-1-(cyanomethyl)-1,2,3,4-tetrahydroquinolin-4-yl]amino}-1-(3,5-difluorobenzyl)-2-hydroxypropyl]acetamide, N-[3-{[1-(cyanomethyl)-6-isopropyl-1,2,3,4-tetrahydroquinolin-4-yl]amino}-1-(3,5-difluorobenzyl)-2-hydroxypropyl]acetamide, N-[3-{[1-(cyanomethyl)-6-isobutyl-1,2,3,4-tetrahydroquinolin-4-yl]amino}-1-(3,5-difluorobenzyl)-2-hydroxypropyl]acetamide, N-[3-{[1-(cyanomethyl)-6-neopentyl-1,2,3,4-tetrahydroquinolin-4-yl]amino}-1-(3,5-difluorobenzyl)-2-hydroxypropyl]acetamide, N-(1-(3,5-difluorobenzyl)-2-hydroxy-3-{[6-(1-hydroxy-2,2-dimethylpropyl)-1,2,3,4-tetrahydroquinolin-4-yl]amino}propyl)acetamide, N-(1-(3,5-difluorobenzyl)-2-hydroxy-.3-{[6-(1-hydroxy-2,2-dimethylpropyl)-1-methyl-1,2,3,4-tetrahydroquinolin-4-yl]amino}propyl)acetamide, N-(1-(3,5-difluorobenzyl)-3-{[2,2-dimethyl-6-neopentyl-1,2,3,4-tetrahydroquinolin-4-yl]amino}-2-hydroxypropyl)acetamide, N-(1-(3,5-difluorobenzyl)-2-hydroxy-3-{[1,2,2-trimethyl-6-neopentyl-1,2,3,4-tetrahydroquinolin-4-yl]amino}propyl)acetamide, N-(1-(3,5-difluorobenzyl)-3-{[1,4-dimethyl-6-neopentyl-1,2,3,4-tetrahydroquinolin-4-yl]amino}-2-hydroxypropyl)acetamide, N-(1-(3,5-difluorobenzyl)-2-hydroxy-3-{[4-methyl-6-neopentyl-1,2,3,4-tetrahydroquinolin-4-yl]amino}propyl)acetamide, N-(1-(3,5-difluorobenzyl)-2-hydroxy-3-{[6-isobutyl-4-methyl-1,2,3,4-tetrahydroquinolin-4-yl]amino}propyl)acetamide, N-(1-(3,5-difluorobenzyl)-2-hydroxy-3-{[6-isobutyl-1,4-dimethyl-1,2,3,4-tetrahydroquinolin-4-yl]amino}propyl)acetamide, N-[3-[(6-tert-butoxy-1,2,3,4-tetrahydroquinolin-4-yl)amino]-1-(3,5-difluorobenzyl)-2-hydroxypropyl]acetamide, N-[3-[(6-tert-butoxy-4-methyl-1,2,3,4-tetrahydroquinolin-4-yl)amino]-1-(3,5-difluorobenzyl)-2-hydroxypropyl]acetamide, N-[3-[(6-tert-butoxy-4,8-dimethyl-1,2,3,4-tetrahydroquinolin-4-yl)amino]-1-(3,5-difluorobenzyl)-2-hydroxypropyl]acetamide, N-{1-(3,5-difluorobenzyl)-2-hydroxy-3-[(4-methyl-6-neopentyl-1,2,3,4-tetrahydroquinolin-4-yl)amino]propyl}acetamide, N-{1-(3,5-difluorobenzyl)-3-[(4,8-dimethyl-6-neopentyl-1,2,3,4-tetrahydroquinolin-4-yl)amino]-2-hydroxypropyl}acetamide, N-{1-(3,5-difluorobenzyl)-2-hydroxy-3-[(8-methyl-6-neopentyl-1,2,3,4-tetrahydroquinolin-4-yl)amino]propyl}acetamide, N-(1-(3,5-difluorobenzyl)-2-hydroxy-3-{[6-(2-hydroxy-2-methylpropyl)-8-methyl-1,2,3,4-tetrahydroquinolin-4-yl]amino}propyl)acetamide, N-(1-(3,5-difluorobenzyl)-2-hydroxy-3-{[6-(2-hydroxy-2-methylpropyl)-4-methyl-1,2,3,4-tetrahydroquinolin-4-yl]amino}propyl)acetamide, N-(1-(3,5-difluorobenzyl)-2-hydroxy-3-{[6-(2-hydroxy-2-methylpropyl)-1,2,3,4-tetrahydroquinolin-4-yl]amino}propyl)acetamide, N-(1-(3,5-difluorobenzyl)-2-hydroxy-3-{[6-(1-hydroxy-2,2-dimethylpropyl)-4-methyl-1,2,3,4-tetrahydroquinolin-4-yl]amino}propyl)acetamide, N-{1-(3,5-difluorobenzyl)-2-hydroxy-3-[(5-isobutyl-2-pyridin-3-ylbenzyl)amino]propyl}acetamide, N-{1-(3,5-difluorobenzyl)-2-hydroxy-3-[(5-isobutyl-2-pyridin-4-ylbenzyl)amino]propyl)acetamide, N-(1-(3,5-difluorobenzyl)-2-hydroxy-3-{[5-isobutyl-2-(6-methoxypyridin-3-yl)benzyl]amino}propyl) acetamide, N-(1-(3,5-difluorobenzyl)-2-hydroxy-3-{[5-isobutyl-2-(5-methoxypyridin-3-yl)benzyl]amino}propyl)acetamide, N-[3-(5,7-Diethyl-1,2,3,4-tetrahydronaphthalen-1-ylamino)-1-(3,5-difluorobenzyl)-2-hydroxypropyl]-acetamide, N-[1-(3,5-Difluorobenzyl)-3-(7-ethyl-5-propyl-1,2,3,4-tetrahydro-naphthalen-1-ylamino)-2-hydroxypropyl]-acetamide, N-[1-(3,5-Difluorobenzyl)-3-(7-ethyl-5-isobutyl-1,2,3,4-tetrahydronaphthalen-1-ylamino)-2-hydroxypropyl]-acetamide, N-{1-(3,5-Difluoro-benzyl)-2-hydroxy-3-(7-pyrimidin-5-yl-1,2,3,4-tetrahydro-naphthalen-1-ylamino)-propyl}-acetamide, N-{1-(3,5-Difluoro-benzyl)-2-hydroxy-3-(7-pyridin-2-yl-1,2,3,4-tetrahydro-naphthalen-1-ylamino)-propyl}-acetamide, N-{1-(3,5-Difluoro-benzyl)-2-hydroxy-3-(7-pyridin-3-yl-1,2,3,4-tetrahydro-naphthalen-1-ylamino)-propyl}-acetamide, N-{1-(3,5-Difluoro-benzyl)-2-hydroxy-3-(7-pyridin-4-yl-1,2,3,4-tetrahydro-naphthalen-1-ylamino)-propyl}-acetamide, N-{1-(3,5-Difluoro-benzyl)-2-hydroxy-3-(7-pyrazin-2-yl-1,2,3,4-tetrahydro-naphthalen-1-ylamino)-propyl}-acetamide, N-{1-(3,5-Difluoro-benzyl)-2-hydroxy-3-[7-(3,6-dimethyl-pyrazin-2-yl)-1,2,3,4-tetrahydro-naphthalen-1-ylamino]-2-hydroxy-propyl}-acetamide, N-{1-(3,5-Difluoro-benzyl)-2-hydroxy-3-(7-furan-2-yl-1,2,3,4-tetrahydro-naphthalen-1-ylamino)-2-hydroxy-propyl}-acetamide, N-{1-(3,5-Difluoro-benzyl)-2-hydroxy-3-(7-thiazol-2-yl-1,2,3,4-tetrahydro-naphthalen-1-ylamino)-propyl}-acetamide, N-{1-(3,5-Difluoro-benzyl)-2-hydroxy-3-(7-thiophen-3-yl-1,2,3,4-tetrahydro-naphthalen-1-ylamino)-propyl}-acetamide, N-{1-(3,5-Difluoro-benzyl)-2-hydroxy-3-(7-styryl-1,2,3,4-tetrahydro-naphthalen-1-ylamino)-propyl}-acetamide, N-{1-(3,5-Difluoro-benzyl)-2-hydroxy-3-[7-(3,5-dimethyl-isoxazol-4-yl)-1,2,3,4-tetrahydro-naphthalen-1-ylamino]-2-hydroxy-propyl}-acetamide, N-{1-(3,5-Difluoro-benzyl)-2-hydroxy-3-(7-thiophen-2-yl-1,2,3,4-tetrahydro-naphthalen-1-ylamino)-propyl}-acetamide, N-{1-(3,5-Difluoro-benzyl)-2-hydroxy-3-[7-(5-ethyl-pyrimidin-2-yl)-1,2,3,4-tetrahydro-naphthalen-1-ylamino]-2-hydroxy-propyl}-acetamide, N-{1-(3,5-Difluoro-benzyl)-2-hydroxy-3-(7-isopropenyl-1,2,3,4-tetrahydro-naphthalen-1-ylamino)-propyl}-acetamide, N-[3-{1-[3-(5-Acetyl-thiophen-2-yl)-phenyl]-cyclopropylamino}-1-(3,5-difluoro-benzyl)-2-hydroxy-propyl]-acetamide, N-{1-(3,5-Difluoro-benzyl)-2-hydroxy-3-[1-(3-thiophen-3-yl-phenyl)-cyclopropylamino]-propyl}-acetamide, N-(1-(3,5-Difluoro-benzyl)-2-hydroxy-3-{1-[3-(6-methoxy-pyridin-3 -yl)-phenyl}-cyclopropylamino}-acetamide, N-{1-(3,5-Difluoro-benzyl)-3-[1-(3-furan-3-yl-phenyl)-cyclopropylamino]-2-hydroxy-propyl}-acetamide, N-(1-(3,5-Difluoro-benzyl)-3-{1-[3-(3,5-dimethyl-isoxazol-4-yl)-phenyl]-cyclopropylamino}-2-hydroxy-propyl)-acetamide, N-[3-(6-tert-Butyl-1,2,3,4-tetrahydro-quinolin-4-ylamino)-1-(3,5-difluoro-benzyl)-2-hydroxy-propyl]-acetamide, N-[3-(6-tert-Butyl-1,2,3,4-tetrahydro-quinolin-4-ylamino)-1-(3-fluoro-benzyl)-2-hydroxy-propyl]-acetamide, N-[1-(3,5-Difluoro-2-methoxy-benzyl)-3-(7-ethyl-1,2,3,4-tetrahydro-naphthalen-1-ylamino)-2-hydroxy-propyl]-acetamide, N-[1-(3,5-Difluoro-benzyl)-2-hydroxy-3-(7-propyl-1,2,3,4-tetrahydro-naphthalen-1-ylamino)-propyl]-2-fluoro-acetamide, N-[1-(3,5-Difluoro-benzyl)-2-hydroxy-3-(1-methyl-7-propyl-1,2,3,4-tetrahydro-naphthalen-1-ylamino)-propyl]-acetamide, N-[3-(7-tert-Butyl-1,2,3,4-tetrahydro-naphthalen-1-ylamino)-1-(3-fluoro-5-hydroxy-benzyl)-2-hydroxy-propyl]-acetamide, N-[1-(3-Benzyloxy-5-fluoro-benzyl)-3-(7-tert-butyl-1,2,3,4-tetrahydro-naphthalen-1-ylamino)-2-hydroxy-propyl]-acetamide, N-[3-{[5-(3-aminophenyl)-7-ethyl-1,2,3,4-tetrahydronaphthalen-1-yl]amino}-1-(3,5-difluorobenzyl)-2-hydroxypropyl]acetamide, N-(1-(3,5-difluorophenyl)-3-hydroxy-4-(7-propyl-1,2,3,4-tetrahydronaphthalen-1-ylamino)butan-2-yl)acetamide, N-(4-(7-tert-butyl-1,2,3,4-tetrahydronaphthalen-1-ylamino)-1-(3-fluoro-4-hydroxyphenyl)-3-hydroxybutan-2-yl)acetamide, N-(1-(3-fluoro-4-hydroxyphenyl)-3-hydroxy-4-(7-neopentyl-1,2,3,4-tetrahydronaphthalen-1-ylamino)butan-2-yl)acetamide, and N-(4-(7-ethyl-1-methyl-1,2,3,4-tetrahydronaphthalen-1-ylamino)-1-(3-fluoro-4-hydroxyphenyl)-3-hydroxybutan-2-yl)acetamide, or a pharmaceutically acceptable salt thereof.

16. The method according to claim 1, wherein the at least one compound of formula (I) is chosen from
N-[1-(3,5-Difluorobenzyl)-3-(7-ethyl-1,2,3,4-tetrahydro-naphthalen-1-ylamino)-2-hydroxy-propyl]-N′, N′-dimethyl-succinamide, Pent-3-enoic acid [1-(3,5-difluoro-benzyl)-3-(7-ethyl-1,2,3,4-tetrahydro-naphthalen-1-ylamino)-2-hydroxy-propyl]-amide, Hex-3-enoic acid [1-(3,5-difluoro-benzyl)-3-(7-ethyl-1,2,3,4-tetrahydro-naphthalen-1-ylamino)-2-hydroxy-propyl]-amide, 3-Allyloxy-N-[1-(3,5-difluoro-benzyl)-3-(7-ethyl-1,2,3,4-tetrahydro-naphthalen-1-ylamino)-2-hydroxy-propyl]-propionamide, N-(1-(3,5-difluorobenzyl)-3-{[7-ethyl-1,2,3,4-tetrahydronaphthalen-1-yl]amino}-2-hydroxypropyl)ethanethioamide hydrochloride, N-[1-(3,5-Difluorobenzyl)-3-(7-ethyl-1,2,3,4-tetrahydro-naphthalen-1-ylamino)-2-hydroxy-propyl]-methanesulfonamide, tert-butyl 1-(3,5-difluorobenzyl)-3-[(6-ethyl-1-methyl-1,2,3,4-tetrahydroquinolin-4-yl)amino]-2-hydroxypropylcarbamate, {1-(3,5-Difluoro-benzyl)-3-[6-(2,2-dimethyl-butyl)-1-methyl-1,2,3,4-tetrahydro-quinolin-4-ylamino]-2-hydroxy-propyl}-carbamic acid tert-butyl ester, {1-(3,5-Difluoro-benzyl)-3-[6-(2,2-dimethyl-propyl)-1-methyl-1,2,3,4-tetrahydro-quinolin-4-ylamino]-2-hydroxy-propyl}-carbamic acid tert-butyl ester, N-[1-(3,5-Difluoro-benzyl)-3-(7-ethyl-1,2,3,4-tetrahydro-naphthalen-1-ylamino)-2-hydroxy-propyl]-2,2-difluoro-acetamide, N-[1-(3,5-Difluoro-benzyl)-3-(7-ethyl-1,2,3,4-tetrahydro-naphthalen-1-ylamino)-2-hydroxy-propyl]-2-hydroxy-acetamide, N-[1-(3,5-Difluorobenzyl)-3-(7-ethyl-1,2,3,4-tetrahydro-naphthalen-1-ylamino)-2-hydroxy-propyl]-propionamide, 5-Oxo-hexanoic acid [1-(3,5-difluorobenzyl)-3-(7-ethyl-1,2,3,4-tetrahydro-naphthalen-1-ylamino)-2-hydroxy-propyl]-amide, N-(1-(3,5-difluorophenyl)-4-(7-ethyl-1,2,3,4-tetrahydronaphthalen-1-ylamino)-3-hydroxybutan-2-yl)methanesulfonamide, N-(1-(3,5-difluorophenyl)-3-hydroxy-4-(7-neopentyl-1,2,3,4-tetrahydronaphthalen-1-ylamino)butan-2-yl)methanesulfonamide, N-(1-(3,5-difluorophenyl)-3-hydroxy-4-(7-neopentyl-1,2,3,4-tetrahydronaphthalen-1-ylamino)butan-2-yl)-3-(methylsulfonamido)benzamide, N-(1-(3,5-difluorophenyl)-3-hydroxy-4-(7-neopentyl-1,2,3,4-tetrahydronaphthalen-1-ylamino)butan-2-yl)-3-(N-methylmethylsulfonamido)benzamide, 2-(3,5-diflubrobenzyl)-4-(7-ethyl-1,2,3,4-tetrahydronaphthalen-1-ylamino)-3-hydroxy-N-methylbutanamide, 2-(3,5-difluoro-2-((methylamino)methyl)benzyl)-4-(7-ethyl-1,2,3,4-tetrahydronaphthalen-1-ylamino)-3-hydroxy-N-methylbutanamide, 4-(7-ethyl-1,2,3,4-tetrahydronaphthalen-1-ylamino)-3-hydroxy-N-methyl-2-((4-propylthiophen-2-yl)methyl)butanamide, and Pentanoic acid [1-(3,5-difluoro-benzyl)-3-(7-ethyl-1,2,3,4-tetrahydro-naphthalen-1-ylamino)-2-hydroxy-propyl]-amide, or a pharmaceutically acceptable salt thereof.

17. The method according to claim 1, wherein the aspartyl protease is beta-secretase and the condition is selected from Alzheimer's disease, Down's syndrome or Trisomy 21, hereditary cerebral hemorrhage with amyloidosis of the Dutch type, chronic inflammation due to amyloidosis, prion diseases, Familial Amyloidotic Polyneuropathy, cerebral amyloid angiopathy, degenerative dementias, dementia associated with Parkinson's disease, dementia associated with progressive supranuclear palsy, and dementia associated with cortical basal degeneration, diffuse Lewy body type of Alzheimer's disease, and frontotemporal dementias with parkinsonism.

18. A method of preventing or treating conditions associated with amyloidosis, comprising:
administering to a host a composition comprising a therapeutically effective amount of at least one compound of formula (I), 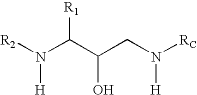
wherein R₁, R₂, and R_{c} are defined as in claim 1, further comprising a composition including beta-secretase complexed with at least one compound of formula (I), or pharmaceutically acceptable salt thereof.

19. A method of preventing or treating the onset of Alzheimer's disease comprising: administering to a patient a therapeutically effective amount of at least one compound of formula (I), 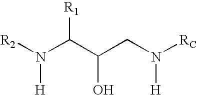
or a pharmaceutically acceptable salt thereof to the patient, wherein R₁, R₂, and R_{c} are defined as in claim 1.

20. A method of preventing or treating the onset of dementia comprising: administering a therapeutically effective amount of at least one compound of formula (I), 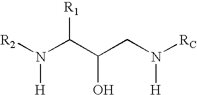
or a pharmaceutically acceptable salt thereof, wherein R₁, R₂, and R_{c} are defined as in claim 1.

21. A method of affecting beta-secretase-mediated cleavage of amyloid precursor protein in a patient, comprising: administering a therapeutically effective amount of at least one compound of formula (I), 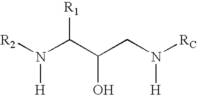
or a pharmaceutically acceptable salt thereof, wherein R₁, R₂, and R_{c} are defined as in claim 1.

22. A method of inhibiting cleavage of amyloid precursor protein at a site between Met596 and Asp597 (numbered for the APP-695 amino acid isotype), or at a corresponding site of an isotype or mutant thereof, comprising: administering a therapeutically effective amount of at least one compound of formula (I), 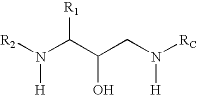
or a pharmaceutically acceptable salt thereof, wherein R₁, R₂, and R_{c} are defined as in claim 1.

23. A method of inhibiting cleavage of amyloid precursor protein or mutant thereof at a site between amino acids, comprising: administering a therapeutically effective amount of at least one compound of formula (I), 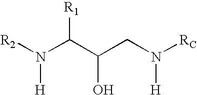
or a pharmaceutically acceptable salt thereof, wherein R₁, R₂, and R_{c} are defined as in claim 1, and wherein the site between amino acids corresponds to
between Met652 and Asp653 (numbered for the APP-751 isotype);
between Met671 and Asp672 (numbered for the APP-770 isotype);
between Leu596 and Asp597 of the APP-695 Swedish Mutation;
between Leu652 and Asp653 of the APP-751 Swedish Mutation; or
between Leu671 and Asp672 of the APP-770 Swedish Mutation.

24. A method of inhibiting production of A-beta, comprising: administering to a patient a therapeutically effective amount of at least one compound of formula (I), 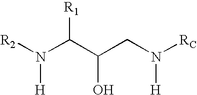
or a pharmaceutically acceptable salt thereof, wherein R₁, R₂, and R_{c} are defined as in claim 1.

25. A method of preventing or treating deposition of A-beta, comprising: administering a therapeutically effective amount of at least one compound of formula (I), 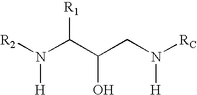
or a pharmaceutically acceptable salt thereof, wherein R₁, R₂, and R_{c} are defined as in claim 1.

26. A method of preventing, delaying, halting, or reversing a disease characterized by A-beta deposits or plaques, comprising: administering a therapeutically effective amount of at least one compound of formula (I), 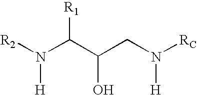
or a pharmaceutically acceptable salt thereof, wherein R₁, R₂, and R_{c} are defined as in claim 1.

27. A method of preventing, delaying, halting, or reversing a condition associated with a pathological form of A-beta in a host comprising: administering to a patient in need thereof an effective amount of at least one compound of formula (I), 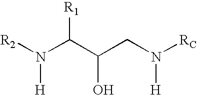
or a pharmaceutically acceptable salt thereof, wherein R₁, R₂, and R_{c} are defined as in claim 1.

28. A method of inhibiting the activity of at least one aspartyl protease in a patient in need thereof, comprising: administering a therapeutically effective amount of at least one compound of formula (I), 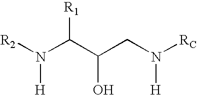
or a pharmaceutically acceptable salt thereof to the patient, wherein R₁, R₂, and R_{c} are defined as in claim 1.

29. The method according to claim 28 wherein the at least one aspartyl protease is beta-secretase.

30. A method of interacting an inhibitor with beta-secretase, comprising: administering to a patient in need thereof a therapeutically effective amount of at least one compound of formula (I), 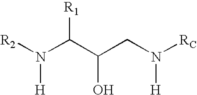
or a pharmaceutically acceptable salt thereof, wherein R₁, R₂, and R_{c} are defined as in claim 1, and wherein the at least one compound interacts with at least one of the following beta-secretase subsites S1, S1′, and S2′.

31. A method of treating a condition in a patient, comprising: administering a therapeutically effective amount of at least one compound of formula (I), 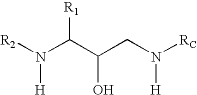
or a pharmaceutically acceptable salt, derivative or biologically active metabolite thereof, to the patient, wherein R₁, R₂, and R_{c} are defined as in claim 1.

32. The method according to claim 31, wherein the condition is selected from Alzheimer's disease, Down's syndrome or Trisomy 21, hereditary cerebral hemorrhage with amyloidosis of the Dutch type, chronic inflammation due to amyloidosis, prion diseases, Familial Amyloidotic Polyneuropathy, cerebral amyloid angiopathy, degenerative dementias, dementia associated with Parkinson's disease, dementia associated with progressive supranuclear palsy, and dementia associated with cortical basal degeneration, diffuse Lewy body type of Alzheimer's disease, and frontotemporal dementias with parkinsonism.

33. A method of modifying the pharmacokinetic parameters of at least one compound of formula (I) 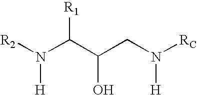
wherein R₁, R₂, and R_{c} are defined as in claim 1, comprising increasing Cₘₐₓ, Tₘₐₓ, and half-life.

34. A method of prescribing a medication for preventing, delaying halting, or reversing disorders, conditions or diseases associated with amyloidosis comprising: identifying in a patient symptoms associated with disorders, conditions or diseases associated with amyloidosis; and prescribing at least one dosage form of at least one compound of formula (I), 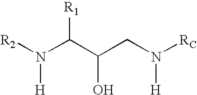
or a pharmaceutically acceptable salt, derivative or biologically active metabolite thereof, to the patient, wherein R₁, R₂, and R_{c} are defined as in claim 1.

35. An article of manufacture, comprising:
(a) at least one dosage form of at least one compound of formula (I), or pharmaceutically acceptable salt thereof, wherein R₁, R₂, and R_{c} are defined as in claim 1;
(b) a package insert providing that a dosage form comprising a compound of formula (I) should be administered to a patient in need of therapy for at least one disorder, condition or disease associated with amyloidosis; and
(c) at least one container in which at least one dosage form of at least one compound of formula (I) is stored.

36. A packaged pharmaceutical composition for treating conditions related to amyloidosis, comprising:
(a) a container which holds an effective amount of at least one compound of formula (I) or a pharmaceutically acceptable salt thereof, wherein R₁, R₂, and R_{c} are defined as in claim 1; and
(b) instructions for using the pharmaceutical composition.

37. An article of manufacture, comprising:
(a) a therapeutically effective amount of at least one compound of formula (I), or pharmaceutically acceptable salt thereof, wherein R₁, R₂, and R_{c} are defined as in claim 1;
(b) a package insert providing an oral dosage form should be administered to a patient in need of therapy for at least one disorder, condition or disease associated with amyloidosis; and
(c) at least one container comprising: at least one oral dosage form of at least one compound of formula (I).

38. An article of manufacture, comprising:
(a) at least one oral dosage form of at least one compound of formula (I), or pharmaceutically acceptable salt thereof, wherein R₁, R₂, and R_{c} are defined as in claim 1, in a dosage amount ranging from about 2 mg to about 1000 mg; associated with (b) a package insert providing that an oral dosage form comprising: a compound of formula (I) in a dosage amount ranging from about 2 mg to about 1000 mg should be administered to a patient in need of therapy for at least one disorder, condition or disease associated with amyloidosis; and
(c) at least one container in which at least one oral dosage form of at least one compound of formula (I) in a dosage amount ranging from about 2 mg to about 1000 mg is stored.

39. An article of manufacture according to claim 38, further comprising: at least one therapeutically active agent stored in the at least one container.

40. The article of manufacture according to claim 38 wherein the therapeutically active agent is selected from an antioxidant, an anti-inflammatory, a gamma-secretase inhibitor, a neurotrophic agent, an acetyl cholinesterase inhibitor, a statin, an A-beta or fragment thereof, and an anti-A-beta antibody.

41. An article of manufacture, comprising:
(a) at least one parenteral dosage form of at least one compound of formula (I), wherein R₁, R₂, and R_{c} are defined as in claim 1, in a dosage amount ranging from about 0.2 mg/mL to about 50 mg/mL; associated with
(b) a package insert providing that a parenteral dosage form comprising: a compound of formula (I) in a dosage amount ranging from about 0.2 mg/mL to about 50 mg/mL should be administered to a patient in need of therapy for at least one disorder, condition or disease associated with amyloidosis; and
(c) at least one container in which at least one parenteral dosage form of at least one compound of formula (I) in a dosage amount ranging from about 0.2 mg/mL to about 50 mg/mL is stored.

42. An article of manufacture comprising:
(a) a medicament comprising: an effective amount of at least one compound of formula (I), in combination with active and/or inactive pharmaceutical agents;
(b) a package insert providing that an effective amount of at least one compound of formula (I) should be administered to a patient in need of therapy for at least one disorder, condition or disease associated with amyloidosis; and
(c) a container in which a medicament comprising: an effective amount of at least one compound of formula (I) in combination with active and/or inactive pharmaceutical agents is stored.

43. A kit comprising:
(a) at least one dosage form of a compound according to claim 1; and
(b) at least one container in which at least one dosage form of a compound according to claim 1 is stored.

44. A kit according to claim 43, further comprising a package insert:
a) containing information of the dosage amount and duration of exposure of a dosage form containing at least one compound of formula (I), and
b) providing that the dosage form should be administered to a patient in need of therapy for at least one disorder, condition or disease associated with amyloidosis.

45. A kit according to claim 44 further comprising: at least one therapeutically active agent.

46. The kit according to claim 45 wherein the therapeutically active agent is selected from an antioxidant, an anti-inflammatory, a gamma-secretase inhibitor, a neurotrophic agent, an acetyl cholinesterase inhibitor, a statin, an A-beta or fragment thereof, and an anti-A-beta antibody.

47. A method of producing a beta-secretase complex comprising: exposing beta-secretase to a compound of formula (I), 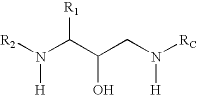
wherein R₁, R₂, and R_{c} are defined as in claim 1, or a pharmaceutically acceptable salt thereof, in a reaction mixture under conditions suitable for the production of the complex.

48. A method of selecting a beta-secretase inhibitor comprising: targeting at least one moiety of a compound of formula (I), 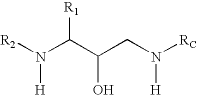
or a pharmaceutically acceptable salt thereof, wherein R₁, R₂, and R_{c} are defined as in claim 1, to interact with at least one of the following beta-secretase subsites S1, S1′, and S2′.

49. A method according to claim 1 wherein the at least one compound of formula (I), 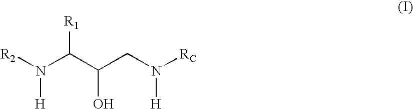
inhibits production of A-beta by at least 10% for a dose of ≦100 mg/kg.

50. The method according to claim 49, wherein the at least one compound of formula (I) is chosen from
N-{1-(3,5-Difluoro-benzyl)-3-[7-(2,2-dimethyl-propyl)-1,2,3,4-tetrahydro-naphthalen-1-ylamino]-2-hydroxy-propyl}-acetamide N-{1-(3,5-Difluoro-benzyl)-3-[7-(2,2-dimethyl-propyl)-5-ethyl-1,2,3,4-tetrahydro-naphthalen-1-ylamino]-2-hydroxy-propyl}-acetamide N-{1-(3,5-Difluoro-benzyl)-3-[7-(2,2-dimethyl-propyl)-1,2,3,4-tetrahydro-naphthalen-1-ylamino]-2-hydroxy-propyl}-2-fluoro-acetamide N-{1-(3,5-Difluoro-benzyl)-3-[6-(2,2-dimethyl-propyl)-1,2,3,4-tetrahydro-quinolin-4-ylamino]-2-hydroxy-propyl}-acetamide, and N-[1-(3,5-Difluoro-benzyl)-3-(7-ethyl-1,2,3,4-tetrahydro-naphthalen-1-ylamino)-2-hydroxy-propyl]-acetamide.

51. The method according to claim 49, wherein the condition is Alzheimer's disease and the at least one aspartyl protease is beta-secretase.

52. The method according to claim 49, wherein the condition is dementia and the at least one aspartyl protease is beta-secretase.

53. A method according to claim 1 wherein the at least one compound of formula (I), 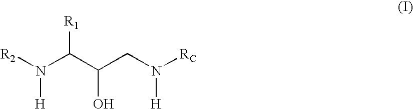
is selective.

54. The method according to claim 53, wherein the at least one compound of formula (I) is chosen from
N-{1-(3,5-Difluoro-benzyl)-3-[6-(2,2-dimethyl-propyl)-1,2,3,4-tetrahydro-quinolin-4-ylamino]-2-hydroxy-propyl}-acetamide, N-{1-(3, 5-Difluoro-benzyl)-3-[7-(2 ,2-dimethyl-propyl)-2-hydroxymethyl-1,2,3,4-tetrahydro-naphthalen-1-ylamino]-2-hydroxy-propyl}-acetamide, and N-[1-(3,5-Difluoro-benzyl)-3-(6-ethyl-1,2,3,4-tetrahydro-quinolin-4-ylamino)-2-hydroxy-propyl]-acetamide.

55. The method according to claim 53, wherein the condition is Alzheimer's disease and the at least one aspartyl protease is beta-secretase.

56. The method according to claim 53, wherein the condition is dementia and the at least one aspartyl protease is beta-secretase.

57. A method according to claim 1 wherein the at least one compound of formula (I), 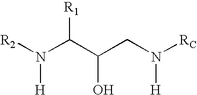
has an F value of at least 10%.

58. The method according to claim 57, wherein the at least one compound of formula (I) is chosen from
N-{1-(3,5-Difluoro-benzyl)-3-[7-(2,2-dimethyl-propyl)-5-ethyl-1,2,3,4-tetrahydro-naphthalen-1-ylamino]-2-hydroxy-propyl}-acetamide, N-{1-(3,5-Difluoro-benzyl)-3-[7-(2,2-dimethyl-propyl)-1,2,3,4-tetrahydro-naphthalen-1-ylamino]-2-hydroxy-propyl}-acetamide, N-{1-(3,5-Difluoro-benzyl)-3-[6-(2,2-dimethyl-propyl)-1,2,3,4-tetrahydro-quinolin-4-ylamino]-2-hydroxy-propyl}-acetamide, and N-[1-(3,5-Difluoro-benzyl)-3-(7-ethyl-1,2,3,4-tetrahydro-naphthalen-1-ylamino)-2-hydroxy-propyl]-acetamide.

59. The method according to claim 57, wherein the condition is Alzheimer's disease and the at least one aspartyl protease is beta-secretase.

60. The method according to claim 57, wherein the condition is dementia and the at least one aspartyl protease is beta-secretase.
